(19) United States
(12) Patent Application Publication
(10) Pub. No.: US 2011/0190514 A1
(43) Pub. Date: Aug. 4, 2011

(54) PYRONE ANALOG COMPOSITIONS AND METHODS

(75) Inventors: Wendye Robbins, San Francisco, CA (US); Ving Lee, Los Altos, CA (US)

(73) Assignee: Limerick BioPharma, Inc., South San Francisco, CA (US)

(21) Appl. No.: 12/877,343

(22) Filed: Sep. 8, 2010

Related U.S. Application Data
(63) Continuation of application No. 12/182,323, filed on Jul. 30, 2008, now abandoned.
(60) Provisional application No. 61/076,584, filed on Jun. 27, 2008.
(60) Provisional application No. 60/953,187, filed on Jul. 31, 2007.

(51) Int. Cl. C07D 311/30

(52) U.S. Cl. 549/400; 549/403

(57) ABSTRACT

Novel pyrone analogs are described which can enhance the effectiveness of a therapeutic agent when administered and/or reduce side effects caused by the administration of a therapeutic agent.

This application claims the benefit of U.S. Provisional Application Ser. Nos. 60/953,187, filed on Jul. 31, 2007 and 61/076,584, filed on Jun. 27, 2008, each of which is hereby incorporated by reference in its entirety.

This application cross references U.S. Provisional Application Ser. Nos. 60/953,186, filed Jul. 31, 2007; 61/076,612, filed Jun. 27, 2008; 60/953,188, filed Jul. 31, 2007; 61/076,608, filed Jun. 27, 2008; 61/076,591, filed Jun. 27, 2008; 61/076, 578, filed Jun. 27, 2008; and 61/076,587, filed on Jun. 27, 2008, each of which is hereby incorporated by reference in its entirety.

### BACKGROUND OF THE INVENTION

Pyrone analogs such as flavonoids have been shown to have beneficial health effects. It has been discovered that pyrone analogs can reduce side effects caused by administration of a therapeutic agent. In another aspect, pyrone analogs can act as blood-tissue barrier (BTB) transport protein modulators, providing beneficial effects such as lowering the side effects of co-administered therapeutic agents.

Certain pyrone analogs have been described in, e.g. U.S. Patent Application Publication Nos. 2006011130 and 20060111308. This application builds upon that work.

### I. SUMMARY OF THE INVENTION

In one aspect of the invention compounds are provided which include pyrone analogs of Formula I:

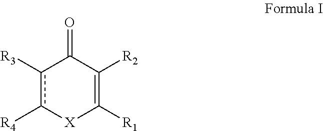

where X is O, S, or NR′ and where R′ is hydrogen, C₁-C₁₀ alkyl, C₂-C₁₀ alkynyl, C₂-C₁₀ alkenyl, C₁-C₁₀ aliphatic acyl, C₆-C₁₀ aromatic acyl, C₆-C₁₀ aralkyl acyl, C₆-C₁₀ alkylaryl acyl, aryl, C₃-C₁₀ heterocyclyl, heteroaryl, or C₃-C₁₀cycloalkyl;

R₁, and R₂ are independently hydrogen, hydroxyl, C₁-C₁₀ alkyl, C₂-C₁₀ alkynyl, C₂-C₁₀ alkenyl, carboxyl, carbohydrate, ester, acyloxy, nitro, halogen, C₁-C₁₀ aliphatic acyl, C₆-C₁₀ aromatic acyl, C₆-C₁₀ aralkyl acyl, C₆-C₁₀ alkylaryl acyl, alkoxy, amine, aryl, C₄-C₁₀heterocyclyl, heteroaryl, C₃-C₁₀cycloalkyl, —OPO₃WY, —OCH₂PO₄WY, —OCH₂PO₄Z or —OPO₃Z;

R₃ and R₄ are independently hydrogen, hydroxyl, C₁-C₁₀ alkyl, C₂-C₁₀ alkynyl, C₂-C₁₀ alkenyl, carboxyl, carbohydrate, ester, acyloxy, nitro, halogen, C₁-C₁₀ aliphatic acyl, C₆-C₁₀ aromatic acyl C₆-C₁₀ aralkyl acyl, C₆-C₁₀ alkylaryl acyl, alkoxy, amine, aryl, C₄-C₁₀heterocyclyl, heteroaryl, C₃-C₁₀cycloalkyl, —OPO₃WY, —OCH₂PO₄WY, —OCH₂PO₄Z or —OPO₃Z;

or R₃ and R₄ are taken together to form a C₅-C₁₀heterocyclyl, C₅-C₁₀cycloalkyl, aryl, or heteroaryl;

W and Y are independently hydrogen, methyl, ethyl, alkyl, carbohydrate, or a cation, and Z is a multivalent cation; and

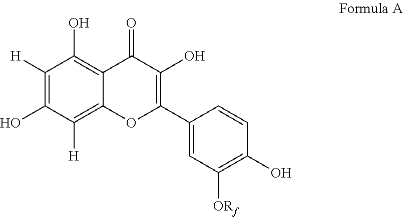

where the compound of Formula I is not the compound of Formula A when R_{f} is hydrogen; and where the compound of Formula I is not one of the following compounds:

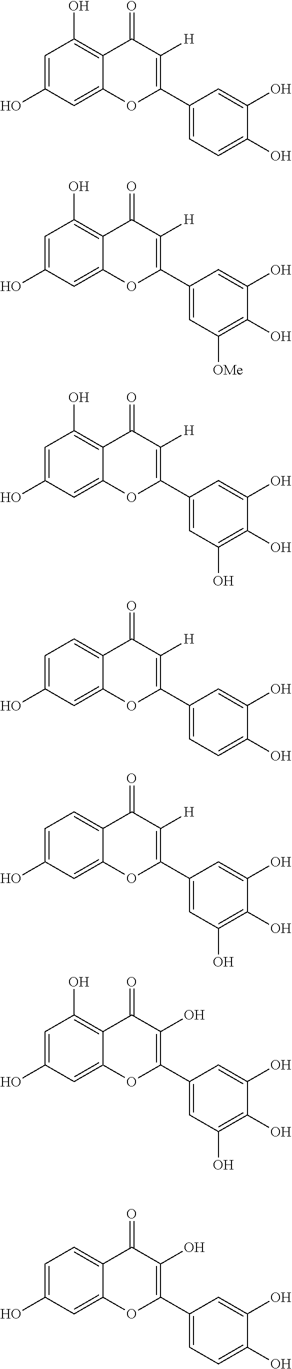

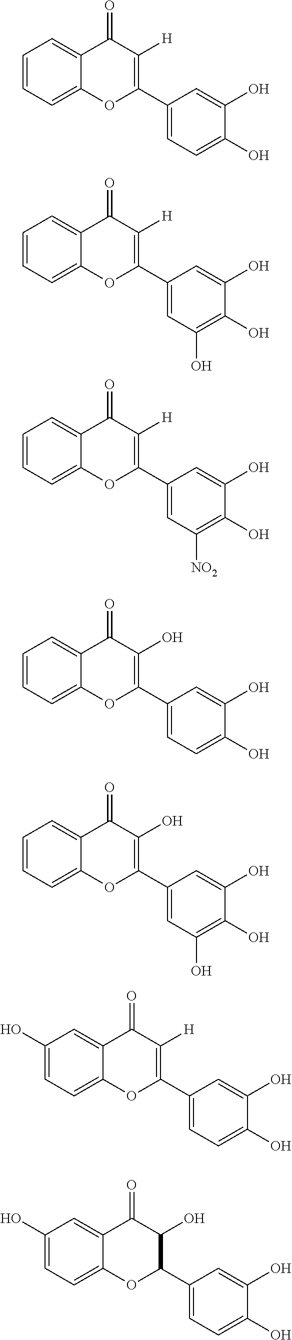

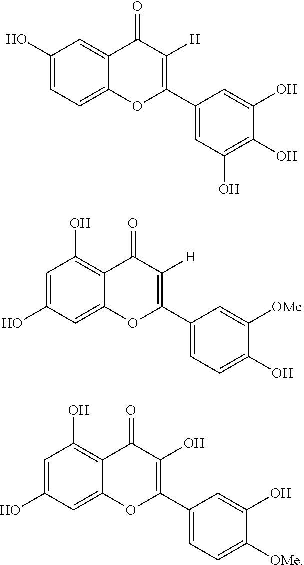

In some embodiments, the compound of Formula I is not the compound of Formula A when R_{f} is —PO₃K₂.

In some embodiments of the invention, the pyrone analog of Formula I is of Formula II:

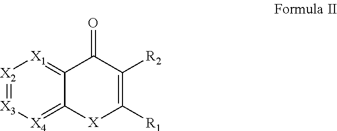

where X₁, X₂, X₃, and X₄ are independently CR₅, O, S, or N; and each instance of R₅ is independently hydrogen, hydroxyl, carboxaldehyde, amino, C₁-C₁₀ alkyl, C₂-C₁₀ alkynyl, C₂-C₁₀ alkenyl, carboxyl, carbohydrate, ester, acyloxy, nitro, halogen, C₁-C₁₀ aliphatic acyl, C₆-C₁₀ aromatic acyl, C₆-C₁₀ aralkyl acyl, C₆-C₁₀ alkylaryl acyl, alkoxy, amine, aryl, C₃-C₁₀heterocyclyl, heteroaryl, C₃-C₁₀cycloalkyl, —OPO₃WY, —OCH₂PO₄WY, —OCH₂PO₄Z or —OPO₃Z.

In some embodiments of the invention, the pyrone analog of Formula II is of Formula III:

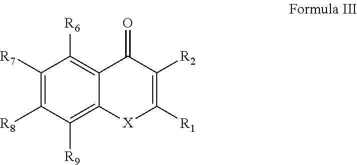

where R₆, R₇, R₈, and R₉ are independently hydrogen, hydroxyl, carboxaldehyde, amino, C₁-C₁₀ alkyl, C₂-C₁₀ alkynyl, C₂-C₁₀ alkenyl, carboxyl, carbohydrate, ester, acyloxy, nitro, halogen, C₁-C₁₀ aliphatic acyl, C₆-C₁₀ aromatic acyl, C₆-C₁₀ aralkyl acyl, C₆-C₁₀alkylaryl acyl, alkoxy, amine, aryl, C₃-C₁₀heterocyclyl, heteroaryl, C₃-C₁₀cycloalkyl, —OPO₃WY, —OCH₂PO₄WY, —OCH₂PO₄Z or —OPO₃Z.

In some embodiments of the invention, the compounds of Formula II are of Formula IV:

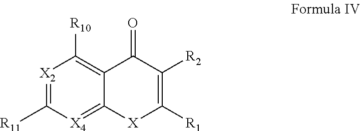

where R₁₀ and R₁₁ are independently hydrogen, hydroxyl, carboxaldehyde, amino, C₁-C₁₀ alkyl, C₂-C₁₀ alkynyl, C₂-C₁₀ alkenyl, carboxyl, carbohydrate, ester, acyloxy, nitro, halogen, C₁-C₁₀ aliphatic acyl, C₆-C₁₀ aromatic acyl, C₆-C₁₀ aralkyl acyl, C₆-C₁₀alkylaryl acyl, alkoxy, amine, aryl, C₃-C₁₀heterocyclyl, heteroaryl, C₃-C₁₀cycloalkyl, —OPO₃WY, —OCH₂PO₄WY, —OCH₂PO₄Z or —OPO₃Z.

In some embodiments of the invention, the compounds of Formula II are of Formula V:

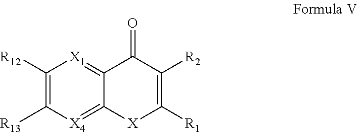

where R₁₂ and R₁₃ are independently hydrogen, hydroxyl, carboxaldehyde, amino, C₁-C₁₀ alkyl, C₂-C₁₀ alkynyl, C₂-C₁₀ alkenyl, carboxyl, carbohydrate, ester, acyloxy, nitro, halogen, C₁-C₁₀ aliphatic acyl, C₆-C₁₀ aromatic acyl, C₆-C₁₀ aralkyl acyl, C₆-C₁₀alkylaryl acyl, alkoxy, amine, aryl, C₃-C₁₀heterocyclyl, heteroaryl, C₃-C₁₀cycloalkyl, —OPO₃WY, —OCH₂PO₄WY, —OCH₂PO₄Z or —OPO₃Z.

In some embodiments of the invention, the pyrone analog of Formula II is of Formula VI:

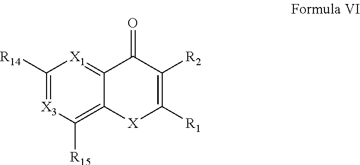

R₁₄ and R₁₅ are independently hydrogen, hydroxyl, carboxaldehyde, amino, C₁-C₁₀ alkyl, C₂-C₁₀ alkynyl, C₂-C₁₀ alkenyl, carboxyl, carbohydrate, ester, acyloxy, nitro, halogen, C₁-C₁₀ aliphatic acyl, C₆-C₁₀ aromatic acyl, C₆-C₁₀ aralkyl acyl, C₆-C₁₀ alkylaryl acyl, alkoxy, amine, aryl, C₃-C₁₀ heterocyclyl, heteroaryl, C₃-C₁₀cycloalkyl, —OPO₃WY, —OCH₂PO₄WY, —OCH₂PO₄Z or —OPO₃Z.

In various embodiments of Formulae I, II, III, IV, V, or VI, R₁ is one of the following formulae:

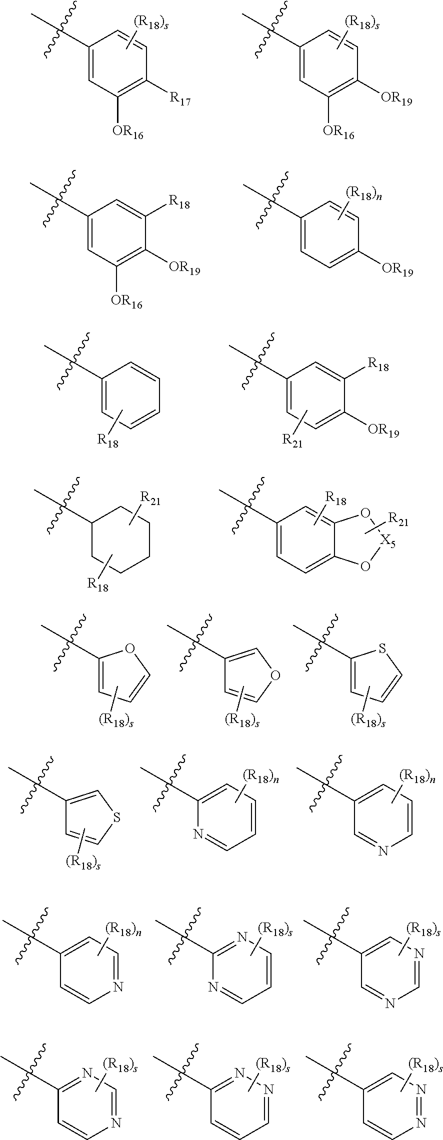

wherein R₁₆ is hydrogen, C₁-C₁₀ alkyl, C₂-C₁₀ alkynyl, C₂-C₁₀ alkenyl, carbohydrate, C₁-C₁₀ aliphatic acyl, C₆-C₁₀ aromatic acyl, C₆-C₁₀ aralkyl acyl, C₆-C₁₀ alkylaryl acyl, aryl, C₃-C₁₀ heterocyclyl, heteroaryl, C₃-C₁₀cycloalkyl, —PO₃WY, —CH₂PO₄WY, —CH₂PO₄Z or —PO₃Z;

R₁₇ is hydrogen, hydroxy, carboxaldehyde, amine, C₁-C₁₀ alkyl, C₂-C₁₀ alkynyl, C₂-C₁₀ alkenyl, carboxyl, carbohydrate, ester, acyloxy, nitro, halogen, C₁-C₁₀ aliphatic acyl, C₆-C₁₀ aromatic acyl, C₆-C₁₀ aralkyl acyl, C₆-C₁₀ alkylaryl acyl, alkoxy, aryl, C₃-C₁₀ heterocyclyl, heteroaryl, or C₃-C₁₀cycloalkyl, —OPO₃WY, —OCH₂PO₄WY, —OCH₂PO₄Z or —OPO₃Z;

each instance of R₁₈ and R₂₁ is independently hydrogen, hydroxyl, carboxaldehyde, amine, C₁-C₁₀ alkyl, C₂-C₁₀ alkynyl, C₂-C₁₀ alkenyl, carboxyl, carbohydrate, ester, acyloxy, nitro, halogen, C₁-C₁₀ aliphatic acyl, C₆-C₁₀ aromatic acyl, C₆-C₁₀ aralkyl acyl, C₆-C₁₀ alkylaryl acyl, alkoxy, alkyl, phosphate, aryl, heteroaryl, C₃-C₁₀ heterocyclic, C₃-C₁₀cycloalkyl, —OPO₃WY, —OCH₂PO₄WY, —OCH₂PO₄Z or —OPO₃Z;

R₁₉ is hydrogen, C₁-C₁₀ alkyl, C₂-C₁₀ alkynyl, C₂-C₁₀ alkenyl, carbohydrate, C₁-C₁₀ aliphatic acyl, C₆-C₁₀ aromatic acyl, C₆-C₁₀ aralkyl acyl, C₆-C₁₀alkylaryl acyl, aryl, C₃-C₁₀heterocyclyl, heteroaryl, optionally substituted C₃-C₁₀cycloalkyl, —PO₃WY, —CH₂PO₄WY, —CH₂PO₄Z or —PO₃Z;

s is an integer of 0, 1, 2, or 3; and

n is an integer of 0, 1, 2, 3, or 4.

In various embodiments of the invention, the pyrone analog of Formula III is of Formula VII:

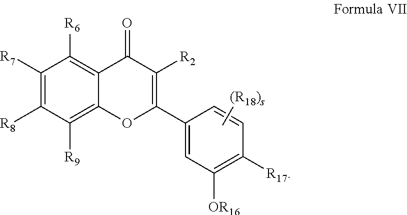

In other embodiments, the compound of Formula III is a compound of Formula VIII:

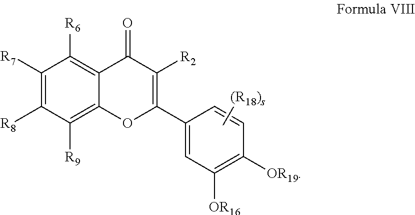

In some embodiments, the compound of Formula III is of Formula IX:

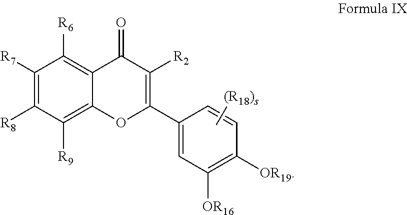

where R₆, R₇, R₈, and R₉ are independently hydrogen, carboxaldehyde, amino, C₁-C₁₀ alkyl, C₂-C₁₀ alkynyl, C₂-C₁₀ alkenyl, carboxyl, carbohydrate, ester, acyloxy, nitro, halogen, C₁-C₁₀ aliphatic acyl, C₆-C₁₀ aromatic acyl, C₆-C₁₀ aralkyl acyl, C₆-C₁₀ alkylaryl acyl, alkoxy, amine, aryl, C₃-C₁₀ heterocyclyl, heteroaryl, C₃-C₁₀cycloalkyl, —OPO₃WY, —OCH₂PO₄WY, —OCH₂PO₄Z or —OPO₃Z.

In some embodiments, the compound of Formula III is of Formula X:

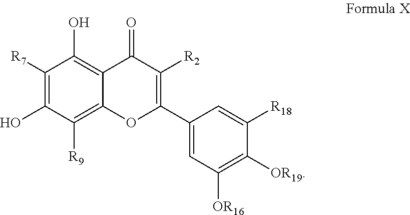

In some embodiments, the compound of Formula III is of Formula XI:

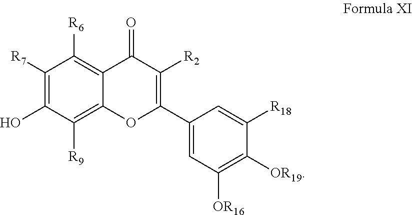

In some embodiments, the compound of Formula III is of Formula XII:

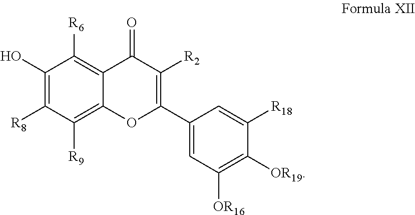

In some embodiments, the compound of Formula III is of Formula XIII:

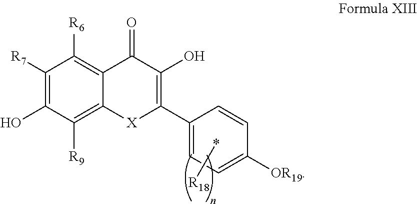

In some embodiments, the compound of Formula III is of Formula XIV:

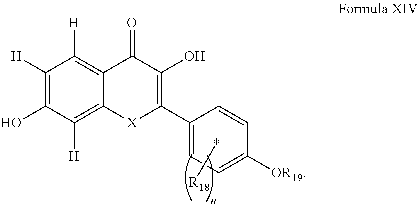

In some embodiments, the compound of Formula III is of Formula XV:

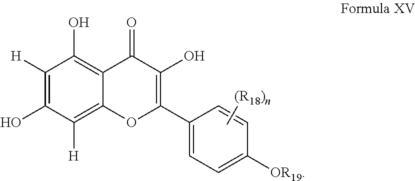

In some embodiments, the compound of Formula III is of Formula XVI:

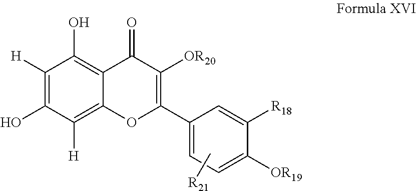

where R₂₀ is independently hydrogen, C₁-C₁₀ alkyl, C₂-C₁₀ alkynyl, C₂-C₁₀ alkenyl, carbohydrate, C₁-C₁₀ aliphatic acyl, C₆-C₁₀ aromatic acyl, C₆-C₁₀ aralkyl acyl, C₆-C₁₀ alkylaryl acyl, aryl, C₃-C₁₀ heterocyclyl, heteroaryl, optionally substituted C₃-C₁₀cycloalkyl, —PO₃WY, —CH₂PO₄WY, —CH₂PO₄Z or —PO₃Z.

In some embodiments, the compound of Formula III is of Formula XVII:

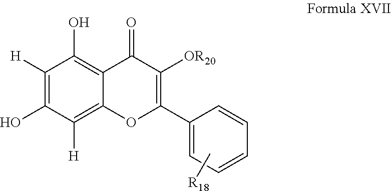

where R₂₀ is hydrogen, C₁-C₁₀ alkyl, C₂-C₁₀ alkynyl, C₂-C₁₀ alkenyl, carbohydrate, C₁-C₁₀ aliphatic acyl, C₆-C₁₀ aromatic acyl, C₆-C₁₀ aralkyl acyl, C₆-C₁₀ alkylaryl acyl, aryl, C₃-C₁₀ heterocyclyl, heteroaryl, optionally substituted C₃-C₁₀cycloalkyl, —PO₃WY, —CH₂PO₄WY, —CH₂PO₄Z or —PO₃Z.

In some embodiments, the compound of Formula III is of Formula XVIII:

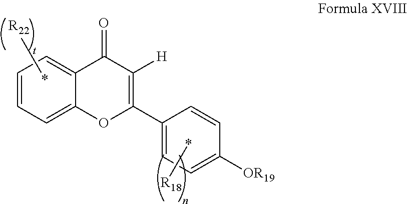

where each instance of R₂₂ is independently hydrogen, hydroxyl, carboxaldehyde, amine, C₁-C₁₀ alkyl, C₂-C₁₀ alkynyl, C₂-C₁₀ alkenyl, carboxyl, carbohydrate, ester, acyloxy, nitro, halogen, C₁-C₁₀ aliphatic acyl, C₆-C₁₀ aromatic acyl, C₆-C₁₀ aralkyl acyl, C₆-C₁₀ alkylaryl acyl, alkoxy, alkyl, phosphate, aryl, heteroaryl, C₃-C₁₀ heterocyclic, C₃-C₁₀cycloalkyl, —OPO₃WY, —OCH₂PO₄WY, —OCH₂PO₄Z or —OPO₃Z; and t is an integer of 0, 1, 2, 3, or 4.

In some embodiments, the compound of Formula III is of Formula XIX:

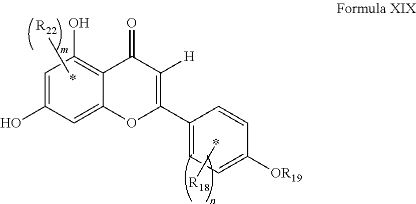

where each instance of R₂₂ is independently hydrogen, hydroxyl, carboxaldehyde, amine, C₁-C₁₀ alkyl, C₂-C₁₀ alkynyl, C₂-C₁₀ alkenyl, carboxyl, carbohydrate, ester, acyloxy, nitro, halogen, C₁-C₁₀ aliphatic acyl, C₆-C₁₀ aromatic acyl, C₆-C₁₀ aralkyl acyl, C₆-C₁₀ alkylaryl acyl, alkoxy, alkyl, phosphate, aryl, heteroaryl, C₃-C₁₀ heterocyclic, C₃-C₁₀cycloalkyl, —OPO₃WY, —OCH₂PO₄WY, —OCH₂PO₄Z or —OPO₃Z; and

m is an integer of 0, 1, or 2.

In some embodiments, the compound of Formula III is of Formula XX:

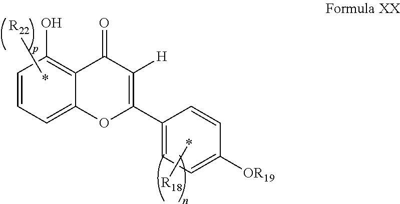

where each instance of R₂₂ is independently hydrogen, hydroxyl, carboxaldehyde, amine, C₁-C₁₀ alkyl, C₂-C₁₀ alkynyl, C₂-C₁₀ alkenyl, carboxyl, carbohydrate, ester, acyloxy, nitro, halogen, C₁-C₁₀ aliphatic acyl, C₆-C₁₀ aromatic acyl, C₆-C₁₀ aralkyl acyl, C₆-C₁₀ alkylaryl acyl, alkoxy, alkyl, phosphate, aryl, heteroaryl, C₃-C₁₀ heterocyclic, C₃-C₁₀cycloalkyl, —OPO₃WY, —OCH₂PO₄WY, —OCH₂PO₄Z or —OPO₃Z; and

p is an integer of 0, 1, 2 or 3.

In some embodiments, the compound of Formula III is of Formula XXI:

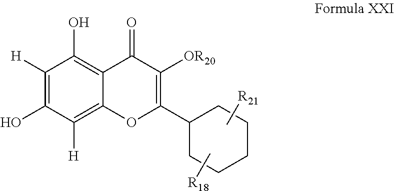

where R₂₁ is hydrogen, hydroxyl, carboxaldehyde, amine, C₁-C₁₀ alkyl, C₂-C₁₀ alkynyl, C₂-C₁₀ alkenyl, carboxyl, carbohydrate, ester, acyloxy, nitro, halogen, C₁-C₁₀ aliphatic acyl, C₆-C₁₀ aromatic acyl, C₆-C₁₀ aralkyl acyl, C₆-C₁₀ alkylaryl acyl, alkoxy, alkyl, phosphate, aryl, heteroaryl, C₃-C₁₀ heterocyclic, C₃-C₁₀cycloalkyl, —OPO₃WY, —OCH₂PO₄WY, —OCH₂PO₄Z or —OPO₃Z; and

R₂₀ is hydrogen, C₁-C₁₀ alkyl, C₂-C₁₀ alkynyl, C₂-C₁₀ alkenyl, carbohydrate, C₁-C₁₀ aliphatic acyl, C₆-C₁₀ aromatic acyl, C₆-C₁₀ aralkyl acyl, C₆-C₁₀alkylaryl acyl, aryl, C₃-C₁₀heterocyclyl, heteroaryl, optionally substituted C₃-C₁₀cycloalkyl, —PO₃WY, —CH₂PO₄WY, —CH₂PO₄Z or —PO₃Z.

In some embodiments, the compound of Formula III is of Formula XXII:

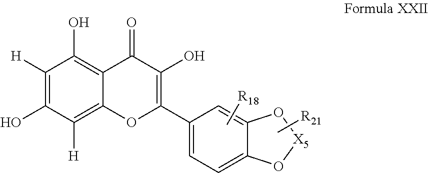

wherein X₅ is a C₁ to C₄ group, optionally interrupted by O, S, NR₂₃, or NR₂₃R₂₃ as valency permits, forming a ring which is aromatic or nonaromatic;

wherein R₂₁ is hydrogen, hydroxyl, carboxaldehyde, amine, C₁-C₁₀ alkyl, C₂-C₁₀ alkynyl, C₂-C₁₀ alkenyl, carboxyl, carbohydrate, ester, acyloxy, nitro, halogen, C₁-C₁₀ aliphatic acyl, C₆-C₁₀ aromatic acyl, C₆-C₁₀ aralkyl acyl, C₆-C₁₀ alkylaryl acyl, alkoxy, alkyl, phosphate, aryl, heteroaryl, C₃-C₁₀ heterocyclic, C₃-C₁₀cycloalkyl, —OPO₃WY, —OCH₂PO₄WY, —OCH₂PO₄Z or —OPO₃Z; and

each instance of R₂₃ is independently hydrogen, C₁-C₁₀ alkyl, C₂-C₁₀ alkynyl, C₂-C₁₀ alkenyl, carbohydrate, acyloxy, C₁-C₁₀ aliphatic acyl, C₆-C₁₀ aromatic acyl, C₆-C₁₀ aralkyl acyl, C₆-C₁₀ alkylaryl acyl, alkoxy, aryl, heteroaryl, C₅-C₁₀heterocyclyl, C₃-C₁₀cycloalkyl, —PO₃WY, —CH₂PO₄WY, —CH₂PO₄Z or —PO₃Z.

In some embodiments, the compound of Formula III is of Formula XXIII:

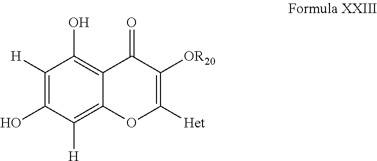

wherein R₂₀ is hydrogen, C₁-C₁₀ alkyl, C₂-C₁₀ alkynyl, C₂-C₁₀ alkenyl, carbohydrate, C₁-C₁₀ aliphatic acyl, C₆-C₁₀ aromatic acyl, C₆-C₁₀ aralkyl acyl, C₆-C₁₀ alkylaryl acyl, aryl, C₃-C₁₀ heterocyclyl, heteroaryl, optionally substituted C₃-C₁₀cycloalkyl, —PO₃WY, —CH₂PO₄WY, —CH₂PO₄Z or —PO₃Z; and

Het is a 3 to 10 membered optionally substituted monocyclic or bicyclic heteroaromatic or heteroalicyclic ring system containing 1, 2, 3, 4, or 5 heteroatoms selected from the group of O, S, and N, with the proviso that no two adjacent ring atoms are O or S, wherein the ring system is unsaturated, partially unsaturated or saturated, wherein any number of the ring atoms have substituents as valency permits which are hydrogen, hydroxyl, carboxyaldehyde, alkylcarboxaldehyde, imino, C₁-C₁₀ alkyl, C₁-C₁₀ alkynyl, C₁-C₁₀ alkenyl, carboxyl, carbohydrate, acyloxy, nitro, halogen, C₁-C₁₀ aliphatic acyl, C₅-C₁₀ aromatic acyl, C₆-C₁₀ aralkyl acyl, C₆-C₁₀ alkylaryl acyl, alkoxy, amine, aryl, heteroaryl, C₅-C₁₀heterocyclyl, C₅-C₁₀cycloalkyl, —OPO₃WY, —OCH₂PO₄WY, —OCH₂PO₄Z or —OPO₃Z.

In some embodiments, the compound of Formula IV is of Formula XXIV or Formula XXV:

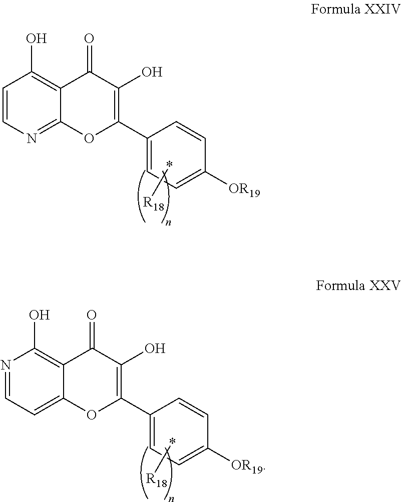

In some embodiments, the compound of Formula IV is of Formula XXVI or Formula XXVII:

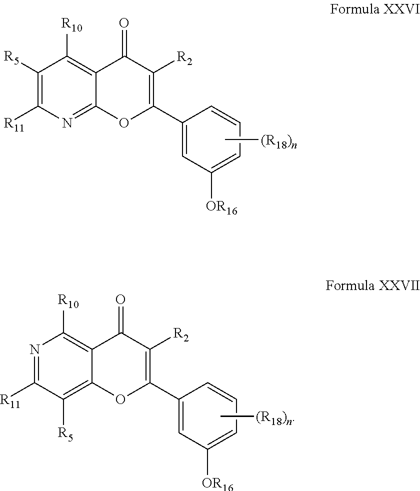

In some embodiments, the compound of Formula IV is of Formula XXVIII:

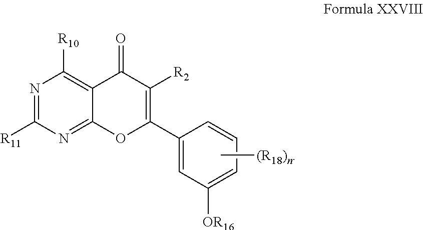

In some embodiments, the compound of Formula V is of Formula XXIX or Formula XXX:

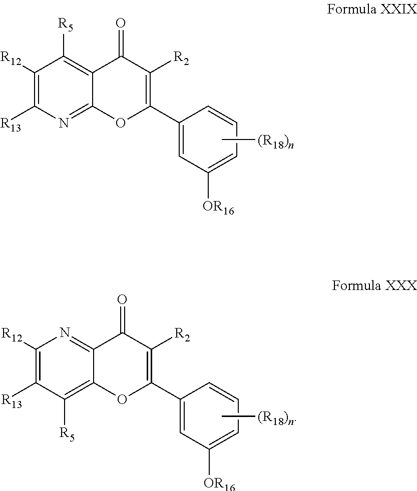

In some embodiments, the compound of Formula V is of Formula XXXI:

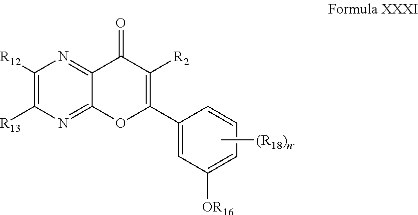

In some embodiments, the compound of Formula VI is of Formula XXXII or Formula XXXIII:

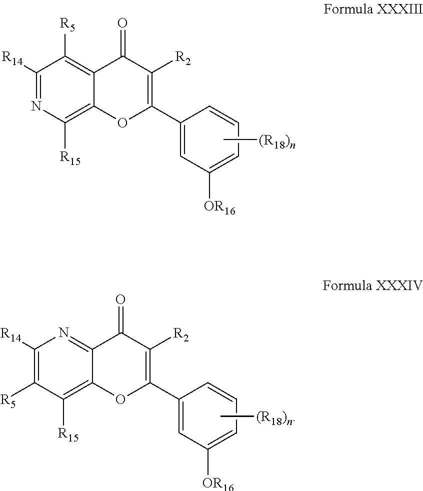

In some embodiments, the compound of Formula VI is of Formula XXXIV:

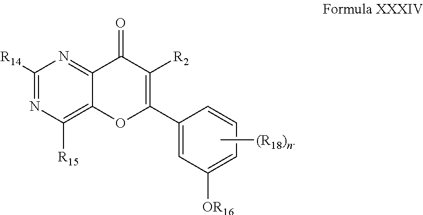

In some embodiments of the invention, R₂ is —H, —OH, —OCH₂CH₃, or —OCH₃.

In other embodiments of the invention, R₅ is —H, —CH₃, —CH₂CH₃, —OCH₂CH₃, —OCH₃ or —Cl.

In yet other embodiments of the invention, R₆ is —H, —CH₃, —CH₂CH₃, —OCH₂CH₃, —OCH₃, or —Cl.

In other embodiments of the invention, R₇ is —H, —CH₃, —CH₂CH₃, —OCH₂CH₃, —OCH₃, or —Cl.

In some embodiments of the invention, R₈ is —H, —CH₃, —CH₂CH₃, —OCH₂CH₃, —OCH₃, —O-glucoronide, or —Cl.

In other embodiments of the invention, R₉ is —H, —OH, —OCH₂CH₃, or —OCH₃.

In some embodiments of the invention, R₁₆ is hydrogen, —PO₃WY, —CH₂PO₄WY, —CH₂PO₄Z or —PO₃Z.

In other embodiments of the invention, R₁₇ is —H, —CH₃, —CH₂CH₃, —OCH₂CH₃, —OCH₃, —PO₃WY, —CH₂PO₄WY, —CH₂PO₄Z or —PO₃Z.

In some embodiments of the invention, R₁₈ is —H, —CH₃, —CH₂CH₃, —OCH₂CH₃, —OCH₃, —Cl, —OPO₃WY, —OCH₂PO₄WY, —OCH₂PO₄Z or —OPO₃Z.

In other embodiments of the invention, R₁₉ is H, CH₃, —CH₂CH₃, —PO₃WY, —CH₂PO₄WY, —CH₂PO₄Z or —PO₃Z.

In some embodiments of the invention, WY is H₂, Na₂, or K₂.

In other embodiments of the invention, Z is Ca or Mg.

In some embodiments of the invention, at least one of R₁₆ and R₁₉ is —PO₃WY, —CH₂PO₄WY, —CH₂PO₄Z or —PO₃Z, or R₁₈ is —OPO₃WY, —OCH₂PO₄WY, —OCH₂PO₄Z or —OPO₃Z.

In another aspect of the invention, a pharmaceutical composition is provided comprising a compound of Formula I and a pharmaceutically acceptable carrier.

### DETAILED DESCRIPTION OF THE INVENTION

Reference will now be made in detail to particularly preferred embodiments of the invention. Examples of the preferred embodiments are illustrated in the following Examples section.

Unless defined otherwise, all technical and scientific terms used herein have the same meaning as is commonly understood by one of skill in the art to which this invention belongs. All patents and publications referred to herein are incorporated by reference.

### Introduction

This invention provides compositions and methods utilizing pyrone analogs and their metabolites. Pyrone analogs, for example, flavonoids can enhance the effectiveness of therapeutic agents, for example, immunosuppressants or analgesics when administered in combination with such agents (see U.S. patent application Ser. Nos. 11/281,771, 11/281,984, and 11/553,924, and “Soluble Pyrone Analogs Methods and Compositions” Attorney Docket No. 31423-716.102, “Phosphorylated Pyrone Analogs and Methods” Attorney Docket No. 31423-720.102, “Methods and Compositions for Therapeutic Treatment” Attorney Docket No. 31423-732.101, “Methods and Compositions for Therapeutic Treatment” Attorney Docket No. 31423-733.101, and “Methods and Compositions for Therapeutic Treatment” Attorney Docket No. 31423-734.101, all of which are herein incorporated by reference in their entirety). Pyrone analogs of the invention may be used to modulate an effect caused by the therapeutic agent; e.g., to treat an animal suffering from a condition (e.g. side effect) caused by administration of a therapeutic agent. In some therapeutic interventions, administration of a therapeutic agent to an animal, which treats a first condition or disorder, causes a side effect, e.g., a second condition or disorder having one or more symptoms distinct from those of the first condition or disorder. This invention also provides novel analogs of these compounds which can have increased effectiveness, e.g., increased modulatory activity, solubility, and/or bioavailability, particularly oral bioavailability. In some embodiments, the compounds act in combination with a therapeutic agent to reduce side effects and/or enhance the effectiveness of the therapeutic agent. The pyrone analogs of the invention may comprise a monocylic or bicyclic nucleus, which may have nitrogen or oxygen substitution within the ring, thus encompassing heteroaromatic compounds in the definition of the aromatic compounds of the invention. These compounds may further comprise an aromatic or heteroaromatic moiety attached to one of the ring carbon atoms. The novel pyrone analogs of the invention may additionally comprise substituents including carbohydrate derivatization on one or more of the hydroxyl moieties. In some embodiments of the invention, the compounds of the present invention can have increased time course of drug effect, in some cases having a longer half life of drug effect, thus prolonging the effects of reducing side effects and/or enhancing the effectiveness of therapeutic agents. In another embodiment of the invention, a carbohydrate derivatized pyrone analog of the invention is metabolized to a glucuronide derivative, which is more active, more bioavailable and/or confers a longer half life of drug effect. In a further embodiment of the invention, the glucuronide derivatized pyrone analog itself is used in the compositions and methods of the invention.

In one aspect, the invention provides compositions and methods utilizing pyrone analogs as agents which reduce or eliminate a side effect of one or more substances. In some embodiments, the invention provides compositions and methods utilizing a combination of a therapeutic agent and a pyrone analog of the invention and/or its metabolite that acts as an agent to reduce or eliminate one or more side effect of the therapeutic agent. Typically, the effect-decreasing agent is a modulator of a blood tissue barrier (BTB) or a placental barrier transport protein. The methods and compositions are useful in the treatment of an animal in need of treatment, where it is desired that one or more effects of a substance, e.g., therapeutic agent, be reduced or eliminated. In embodiments further utilizing a therapeutic agent, the methods and compositions are useful in the treatment of an animal in need of treatment, where it is desired that one or more side effects of the therapeutic agent be reduced or eliminated while one or more of the therapeutic effects (e.g., peripheral effects) of the agent are retained or enhanced.

In some embodiments of the invention, the therapeutic agent is an immunosuppressant, such as a calcineurin inhibitor, e.g., cyclosporine, tacrolimus, or a tacrolimus analog. In some embodiments of the invention, the therapeutic agent is an analgesic agent, such as an opiate or a non-opiate analgesic. In some embodiments of the invention, the therapeutic agent is a non-analgesic agent. The polyphenol and/or its metabolite acting as an agent causing a decrease in the side effects of the therapeutic agent, e.g., a modulator of a BTB transport protein may be an activator or an inhibitor of the protein. The modulatory effect may be dose-dependent, e.g., some modulators act as activators in one dosage range and inhibitors in another. In some embodiments, a modulator of a BTB transport protein is used in a dosage wherein it acts primarily as an activator.

Typically, the use of a pyrone analog of the invention and/or its metabolite, results in a decrease in one or more side effects of the therapeutic agent. The therapeutic effect(s) of the agent may be decreased, remain the same, or increase; however, in preferred embodiments, if the therapeutic effect is decreased, it is not decreased to the same degree as the side effects. It will be appreciated that a given therapeutic agent may have more than one therapeutic effect and or one or more side effects, and it is possible that the therapeutic ratio (in this case, the ratio of change in desired effect to change in undesired effect) may vary depending on which effect is measured. However, in some embodiments, at least one therapeutic effect of the therapeutic agent is decreased to a lesser degree than at least one side effect of the therapeutic agent.

In addition, in some embodiments, one or more therapeutic effects of the agent is enhanced by use in combination with a pyrone analog of the invention and/or its metabolite acting, optionally while one or more side effects of the therapeutic agent is reduced or substantially eliminated. For example, in some embodiments, the analgesic effect of an analgesic agent is enhanced while one or more CNS effects of the agent is reduced or substantially eliminated.

Without being bound by theory, and as an example only of a possible mechanism, it is thought that the methods and compositions of the invention operate by reducing or eliminating the concentration of the therapeutic agent from one or more compartments of the body, while retaining or even increasing the effective concentration of the agent in the other compartments.

A pyrone analog of the invention may be converted in vivo to metabolites that have differing activities in the modulation of one or more BTB transport modulators, and these metabolites are also encompassed by the compositions and methods of the invention.

### II. PYRONE ANALOGS OF THE INVENTION

As used herein and in the appended claims, the singular forms “a,” “and,” and “the” include plural referents unless the context clearly dictates otherwise. Thus, for example, reference to “a compound” includes a plurality of such compounds, and reference to “the cell” includes reference to one or more cells (or to a plurality of cells) and equivalents thereof known to those skilled in the art, and so forth. When ranges are used herein for physical properties, such as molecular weight, or chemical properties, such as chemical formulae, all combinations and subcombinations of ranges and specific embodiments therein are intended to be included. The term “about” when referring to a number or a numerical range means that the number or numerical range referred to is an approximation within experimental variability (or within statistical experimental error), and thus the number or numerical range may vary between 1% and 15% of the stated number or numerical range. The term “comprising” (and related terms such as “comprise” or “comprises” or “having” or “including”) is not intended to exclude that in other certain embodiments, for example, an embodiment of any composition of matter, composition, method, or process, or the like, described herein, may “consist of” or “consist essentially of” the described features.

Compounds useful in the invention include compounds having a general formula corresponding to Formula Q, where the numbering of atom centers is as shown, and is used throughout the specification. The numbering is retained even for analogs with variant structures, i.e., heteroatom substitution.

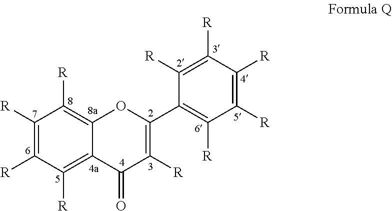

“Acyl” refers to a —(C═O)— radical which is attached to two other moieties through the carbon atom. Those groups may be chosen from alkyl alkenyl, alkynyl, aryl, heterocylic, heteroaliphatic, heteroaryl, and the like. Unless stated otherwise specifically in the specification, an acyl group is optionally substituted by one or more substituents which independently are: halo, cyano, nitro, oxo, thioxo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —N(R^{a})C(O)R^{a}, —N(R^{a})S(O)ₜR^{a} (where t is 1 or 2), —S(O)ₜOR^{a} (where t is 1 or 2), —S(O)ₜN(R^{a})₂ (where t is 1 or 2), —OPO₃WY (where W and Y are hydrogen, methyl, ethyl, alkyl carbohydrate, lithium, sodium or potassium) or —OPO₃Z (where Z is calcium, magnesium or iron) where each R^{a} is independently hydrogen, alkyl fluoroalkyl, carbocyclyl, carbocyclylalkyl, aryl, aralkyl, heterocyclyl, heterocyclylalkyl, heteroaryl or heteroarylalkyl.

“Acyloxy” refers to a R(C═O)O— radical wherein R is alkyl aryl, heteroaryl or heterocyclyl. Unless stated otherwise specifically in the specification, an acyloxy group is optionally substituted by one or more substituents which independently are: halo, cyano, nitro, oxo, thioxo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —N(R^{a})C(O)R^{a}, —N(R^{a})S(O)ₜR^{a} (where t is 1 or 2), —S(O)ₜOR^{a} (where t is 1 or 2) —S(O)ₜN(R^{a})₂ (where t is 1 or 2), —OPO₃WY (where W and Y are hydrogen, methyl, ethyl, alkyl carbohydrate, lithium, sodium or potassium) or —OPO₃Z (where Z is calcium, magnesium or iron) where each R^{a} is independently hydrogen, alkyl fluoroalkyl, carbocyclyl, carbocyclylalkyl, aryl, aralkyl, heterocyclyl, heterocyclylalkyl, heteroaryl or heteroarylalkyl.

“Alkylaryl” refers to an (alkyl)aryl radical, where alkyl and aryl are as defined herein.

“Aralkyl” refers to an (aryl)alkyl-radical where aryl and alkyl are as defined herein.

“Alkoxy” refers to a (alkyl)O radical, where alkyl is as described herein and contains 1 to 10 carbons (e.g., C₁-C₁₀ alkyl). Whenever it appears herein, a numerical range such as “1 to 10” refers to each integer in the given range; e.g., “1 to 10 carbon atoms” means that the alkyl group may consist of 1 carbon atom, 2 carbon atoms, 3 carbon atoms, etc., up to and including 10 carbon atoms. In some embodiments, it is a C₁-C₄ alkoxy group. A alkoxy moiety is optionally substituted by one or more of the substituents described as suitable substituents for an alkyl radical.

“Alkyl” refers to a straight or branched hydrocarbon chain radical consisting solely of carbon and hydrogen atoms, containing no unsaturation, having from one to ten carbon atoms (e.g., C₁-C₁₀ alkyl). Whenever it appears herein, a numerical range such as “1 to 10” refers to each integer in the given range; e.g., “1 to 10 carbon atoms” means that the alkyl group may consist of 1 carbon atom, 2 carbon atoms, 3 carbon atoms, etc., up to and including 10 carbon atoms, although the present definition also covers the occurrence of the term “alkyl” where no numerical range is designated. Typical alkyl groups include, but are in no way limited to, methyl, ethyl, propyl, isopropyl, n-butyl, iso-butyl, sec-butyl isobutyl, tertiary butyl, pentyl, isopentyl, neopentyl, hexyl, septyl, octyl, nonyl, decyl, and the like. The alkyl is attached to the rest of the molecule by a single bond, for example, methyl (Me), ethyl (Et), n-propyl, i-methylethyl (iso-propyl), n-butyl, n-pentyl, 1,1-dimethylethyl (t-butyl), 3-methylhexyl, 2-methylhexyl, and the like. Unless stated otherwise specifically in the specification, an alkyl group is optionally substituted by one or more substituents which independently are: halo, cyano, nitro, oxo, thioxo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —N(R^{a})C(O)R^{a}, —N(R^{a})S(O)ₜR^{a} (where t is 1 or 2), —S(O)ₜOR^{a} (where t is 1 or 2), —S(O)ₜN(R^{a})₂ (where t is 1 or 2), —OPO₃WY (where W and Y are hydrogen, methyl, ethyl, alkyl, carbohydrate, lithium, sodium or potassium) or —OPO₃Z (where Z is calcium, magnesium or iron) where each R^{a} is independently hydrogen, alkyl, fluoroalkyl, carbocyclyl, carbocyclylalkyl, aryl, aralkyl, heterocyclyl, heterocyclylalkyl, heteroaryl or heteroarylalkyl.

An “alkene” moiety refers to a group consisting of at least two carbon atoms and at least one carbon-carbon double bond, and an “alkyne” moiety refers to a group consisting of at least two carbon atoms and at least one carbon-carbon triple bond. The alkyl moiety, whether saturated or unsaturated, may be branched, straight chain, or cyclic.

“Alkenyl” refers to a straight or branched hydrocarbon chain radical group consisting solely of carbon and hydrogen atoms, containing at least one double bond, and having from two to ten carbon atoms (i.e. C₂-C₁₀ alkenyl). Whenever it appears herein, a numerical range such as “2 to 10” refers to each integer in the given range; e.g., “2 to 10 carbon atoms” means that the alkenyl group may consist of 2 carbon atoms, 3 carbon atoms, etc., up to and including 10 carbon atoms. In certain embodiments, an alkenyl comprises two to eight carbon atoms. In other embodiments, an alkenyl comprises two to four carbon atoms. The alkenyl is attached to the rest of the molecule by a single bond, for example, ethenyl (i.e., vinyl), prop-1-enyl (i.e., allyl), but-1-enyl, pent-1-enyl, penta-1,4-dienyl, and the like. Unless stated otherwise specifically in the specification, an alkenyl group is optionally substituted by one or more substituents which independently are: halo, cyano, nitro, oxo, thioxo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —N(R^{a})C(O)R^{a}, —N(R^{a})S(O)ₜR^{a} (where t is 1 or 2), —S(O)ₜOR^{a} (where t is 1 or 2), —S(O)ₜN(R^{a})₂ (where t is 1 or 2), —OPO₃WY (where W and Y are hydrogen, methyl, ethyl, alkyl, carbohydrate, lithium, sodium or potassium) or —OPO₃Z (where Z is calcium, magnesium or iron) where each R^{a} is independently hydrogen, alkyl, fluoroalkyl, carbocyclyl, carbocyclylalkyl, aryl, aralkyl, heterocyclyl, heterocyclylalkyl, heteroaryl or heteroarylalkyl.

“Alkynyl” refers to a straight or branched hydrocarbon chain radical group consisting solely of carbon and hydrogen atoms, containing at least one triple bond, having from two to ten carbon atoms (i.e. C₂-C₁₀ alkynyl). Whenever it appears herein, a numerical range such as “2 to 10” refers to each integer in the given range; e.g., “2 to 10 carbon atoms” means that the alkynyl group may consist of 2 carbon atoms, 3 carbon atoms, etc., up to and including 10 carbon atoms. In certain embodiments, an alkynyl comprises two to eight carbon atoms. In other embodiments, an alkynyl has two to four carbon atoms. The alkynyl is attached to the rest of the molecule by a single bond, for example, ethynyl, propynyl, butynyl, pentynyl, hexynyl, and the like. Unless stated otherwise specifically in the specification, an alkynyl group is optionally substituted by one or more substituents which independently are: halo, cyano, nitro, oxo, thioxo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —N(R^{a})C(O)R^{a}, —N(R^{a})S(O)ₜR^{a} (where t is 1 or 2), —S(O)ₜOR^{a} (where t is 1 or 2), —S(O)ₜN(R^{a})₂ (where t is 1 or 2), —OPO₃WY (where W and Y are hydrogen, methyl, ethyl, alkyl, carbohydrate, lithium, sodium or potassium) or —OPO₃Z (where Z is calcium, magnesium or iron) where each R^{a} is independently hydrogen, alkyl, fluoroalkyl, carbocyclyl, carbocyclylalkyl, aryl, aralkyl, heterocyclyl, heterocyclylalkyl, heteroaryl or heteroarylalkyl.

“Amine” refers to a —N(R^{a})₂ radical group, where each R^{a} is independently hydrogen, alkyl, fluoroalkyl, carbocyclyl, carbocyclylalkyl, aryl, aralkyl, heterocyclyl, heterocyclylalkyl, heteroaryl or heteroarylalkyl, unless stated otherwise specifically in the specification. Unless stated otherwise specifically in the specification, an amino group is optionally substituted by one or more substituents which independently are: halo, cyano, nitro, oxo, thioxo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —N(R^{a})C(O)R^{a}, —N(R^{a})S(O)ₜR^{a} (where t is 1 or 2), —S(O)ₜOR^{a} (where t is 1 or 2), —S(O)ₜN(R^{a})₂ (where t is 1 or 2), —OPO₃WY (where W and Y are hydrogen, methyl, ethyl, alkyl, carbohydrate, lithium, sodium or potassium) or —OPO₃Z (where Z is calcium, magnesium or iron) where each R^{a} is independently hydrogen, alkyl, fluoroalkyl, carbocyclyl, carbocyclylalkyl, aryl, aralkyl, heterocyclyl, heterocyclylalkyl, heteroaryl or heteroarylalkyl.

An “amide” refers to a chemical moiety with formula —C(O)NHR or —NHC(O)R, where R is selected from the group consisting of alkyl, cycloalkyl, aryl, heteroaryl (bonded through a ring carbon) and heteroalicyclic (bonded through a ring carbon). An amide may be an amino acid or a peptide molecule attached to a compound of Formula (I), thereby forming a prodrug. Any amine, hydroxy, or carboxyl side chain on the compounds described herein can be amidified. The procedures and specific groups to make such amides are known to those of skill in the art and can readily be found in reference sources such as Greene and Wuts, Protective Groups in Organic Synthesis, 3.sup.rd Ed., John Wiley & Sons, New York, N.Y., 1999, which is incorporated herein by reference in its entirety.

“Aromatic” or “aryl” refers to an aromatic radical with six to ten ring atoms (e.g., C₆-C₁₀ aromatic or C₆-C₁₀ aryl) which has at least one ring having a conjugated pi electron system and includes both carbocyclic aryl (e.g., phenyl, fluorenyl, and naphthyl) and heterocyclic aryl (or “heteroaryl” or “heteroaromatic”) groups (e.g., pyridine). Whenever it appears herein, a numerical range such as “6 to 10” refers to each integer in the given range; e.g., “6 to 10 ring atoms” means that the aryl group may consist of 6 ring atoms, 7 ring atoms, etc., up to and including 10 ring atoms. The term includes monocyclic or fused-ring polycyclic (i.e., rings which share adjacent pairs of ring atoms) groups. Unless stated otherwise specifically in the specification, an aryl moiety is optionally substituted by one or more substituents which are independently: hydroxyl, carboxaldehyde, amine, C₁-C₁₀ alkyl, C₂-C₁₀ alkynyl, C₂-C₁₀ alkenyl, carboxyl, carbohydrate, ester, acyloxy, nitro, halogen, C₁-C₁₀ aliphatic acyl, C₆-C₁₀ aromatic acyl, C₆-C₁₀ aralkyl acyl, C₆-C₁₀ alkylaryl acyl, alkoxy, alkyl, phosphate, aryl, heteroaryl, C₃-C₁₀ heterocyclic, C₃-C₁₀cycloalkyl, —CN—OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —N(R^{a})C(O)R^{a}, —N(R^{a})S(O)ₜR^{a} (where t is 1 or 2), —S(O)ₜOR^{a} (where t is 1 or 2), —S(O)ₜN(R^{a})₂ (where t is 1 or 2), —OPO₃WY (where W and Y are hydrogen, methyl, ethyl, alkyl, carbohydrate, lithium, sodium or potassium) or —OPO₃Z (where Z is calcium, magnesium or iron) where each R^{a} is independently hydrogen, alkyl, fluoroalkyl, carbocyclyl, carbocyclylalkyl, aryl, aralkyl, heterocyclyl, heterocyclylalkyl, heteroaryl or heteroarylalkyl.

“Carboxaldehyde” refers to a —(C═O)H radical.

“Carboxyl” refers to a —(C═O)OH radical.

“Carbohydrate” as used herein, includes, but not limited to, monosaccharides, disaccharides, oligosaccharides, or polysaccharides. Monosaccharide for example includes, but not limited to, aldotrioses such as glyceraldehyde, ketotrioses such as dihydroxyacetone, aldotetroses such as erythrose and threose, ketotetroses such as erythrulose, aldopentoses such as arabinose, lyxose, ribose and xylose, ketopentoses such as ribulose and xylulose, aldohexoses such as allose, altrose, galactose, glucose, gulose, idose, mannose and talose, ketohexoses such as fructose, psicose, sorbose and tagatose, heptoses such as mannoheptulose, sedoheptulose, octoses such as octolose, 2-keto-3-deoxy-manno-octonate, nonoses such as sialoseallose. Disaccharides for example includes, but not limited to, glucorhamnose, trehalose, sucrose, lactose, maltose, galactosucrose, N-acetyllactosamine, cellobiose, gentiobiose, isomaltose, melibiose, primeverose, hesperodinose, and rutinose. Oligosaccharides for example includes, but not limited to, raffinose, nystose, panose, cellotriose, maltotriose, maltotetraose, xylobiose, galactotetraose, isopanose, cyclodextrin (α-CD) or cyclomaltohexaose, β-cyclodextrin (β-CD) or cyclomaltoheptaose and γ-cyclodextrin (γ-CD) or cyclomaltooctaose. Polysaccharide for example includes, but not limited to, xylan, mannan, galactan, glucan, arabinan, pustulan, gellan, guaran, xanthan, and hyaluronan. Some examples include, but not limited to, starch, glycogen, cellulose, inulin, chitin, amylose and amylopectin.

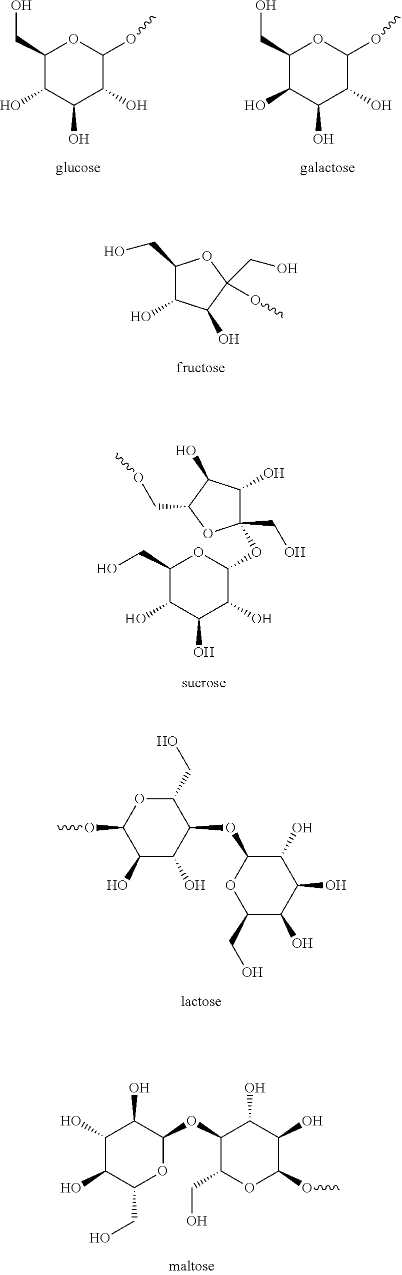

A compound of Formula I having a carbohydrate moeity can be referred to as the pyrone aromatic glycoside or the pyrone aromatic saccharide. As used herein, “carbohydrate” further encompasses the glucuronic as well as the glycosidic derivative of compounds of Formula I. Where the pyrone analog has no carbohydrate moeity, it can be referred to as the aglycone. Further, where a phenolic hydroxy is derivatized with any of the carbohydrates described above, the carbohydrate moiety is referred to as a glycosyl residue. Unless stated otherwise specifically in the specification, a carbohydrate group is optionally substituted by one or more substituents which are independently: halo, cyano, nitro, oxo, thioxo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —N(R^{a})C(O)R^{a}, —N(R^{a})S(O)ₜR^{a} (where t is 1 or 2), —S(O)ₜOR^{a} (where t is 1 or 2), —S(O)ₜN(R^{a})₂ (where t is 1 or 2), —OPO₃WY (where W and Y are hydrogen, methyl, ethyl, alkyl, carbohydrate, lithium, sodium or potassium) or —OPO₃Z (where Z is calcium, magnesium or iron) where each R^{a} is independently hydrogen, alkyl, fluoroalkyl, carbocyclyl, carbocyclylalkyl, aryl, aralkyl, heterocyclyl, heterocyclylalkyl, heteroaryl or heteroarylalkyl.

“Cyano” refers to a —CN moiety.

“Cycloalkyl” refers to a monocyclic or polycyclic radical that contains only carbon and hydrogen, and may be saturated, partially unsaturated, or fully unsaturated. Cycloalkyl groups include groups having from 3 to 10 ring atoms (i.e. C₂-C₁₀ cycloalkyl). Whenever it appears herein, a numerical range such as “3 to 10” refers to each integer in the given range; e.g., “3 to 10 carbon atoms” means that the cycloalkyl group may consist of 3 carbon atoms, etc., up to and including 10 carbon atoms. Illustrative examples of cycloalkyl groups include, but are not limited to the following moieties: cyclopropyl, cyclobutyl, cyclopentyl, cyclohexyl, cycloseptyl, cyclooctyl, cyclononyl, cyclodecyl, norbornyl, and the like. Unless stated otherwise specifically in the specification, a cycloalkyl group is optionally substituted by one or more substituents which are independently: halo, cyano, nitro, oxo, thioxo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —N(R^{a})C(O)R^{a}, —N(R^{a})S(O)ₜR^{a} (where t is 1 or 2), —S(O)ₜOR^{a} (where t is 1 or 2), —S(O)ₜN(R^{a})₂ (where t is 1 or 2), —OPO₃WY (where W and Y are hydrogen, methyl, ethyl, alkyl, carbohydrate, lithium, sodium or potassium) or —OPO₃Z (where Z is calcium, magnesium or iron) where each R^{a} is independently hydrogen, alkyl, fluoroalkyl, carbocyclyl, carbocyclylalkyl, aryl, aralkyl, heterocyclyl, heterocyclylalkyl, heteroaryl or heteroarylalkyl.

“Ester” refers to a chemical radical of formula —COOR, where R is selected from the group consisting of alkyl, cycloalkyl, aryl, heteroaryl (bonded through a ring carbon) and heteroalicyclic (bonded through a ring carbon). Any amine, hydroxy, or carboxyl side chain on the compounds described herein can be esterified. The procedures and specific groups to make such esters are known to those of skill in the art and can readily be found in reference sources such as Greene and Wuts, Protective Groups in Organic Synthesis, 3.sup.rd Ed., John Wiley & Sons, New York, N.Y., 1999, which is incorporated herein by reference in its entirety. Unless stated otherwise specifically in the specification, an ester group is optionally substituted by one or more substituents which are independently: halo, cyano, nitro, oxo, thioxo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —N(R^{a})C(O)R^{a}, —N(R^{a})S(O)ₜR^{a} (where t is 1 or 2), —S(O)ₜOR^{a} (where t is 1 or 2), —S(O)ₜN(R^{a})₂ (where t is 1 or 2), —OPO₃WY (where W and Y are hydrogen, methyl, ethyl, alkyl, carbohydrate, lithium, sodium or potassium) or —OPO₃Z (where Z is calcium, magnesium or iron) where each R^{a} is independently hydrogen, alkyl, fluoroalkyl, carbocyclyl, carbocyclylalkyl, aryl, aralkyl, heterocyclyl, heterocyclylalkyl, heteroaryl or heteroarylalkyl.

“Fluoroalkyl” refers to an alkyl radical, as defined above, that is substituted by one or more fluoro radicals, as defined above, for example, trifluoromethyl, difluoromethyl, 2,2,2-trifluoroethyl, 1-fluoromethyl-2-fluoroethyl, and the like. The alkyl part of the fluoroalkyl radical may be optionally substituted as defined above for an alkyl group.

“Halo”, “halide”, or, alternatively, “halogen” means fluoro, chloro, bromo or iodo. The terms “haloalkyl,” “haloalkenyl,” “haloalkynyl” and “haloalkoxy” include alkyl, alkenyl, alkynyl and alkoxy structures that are substituted with one or more halo groups or with combinations thereof. For example, the terms “fluoroalkyl” and “fluoroalkoxy” include haloalkyl and haloalkoxy groups, respectively, in which the halo is fluorine.

The terms “heteroalkyl” “heteroalkenyl” and “heteroalkynyl” include optionally substituted alkyl, alkenyl and alkynyl radicals and which have one or more skeletal chain atoms selected from an atom other than carbon, e.g., oxygen, nitrogen, sulfur, phosphorus or combinations thereof.

“Heteroaryl” or, alternatively, “heteroaromatic” refers to a 5- to 18-membered aryl group (e.g., C₅-C₁₃ heteroaryl) that includes one or more ring heteroatoms selected from nitrogen, oxygen and sulfur, and which may be a monocyclic, bicyclic, tricyclic or tetracyclic ring system. Whenever it appears herein, a numerical range such as “5 to 18” refers to each integer in the given range; e.g., “5 to 18 ring atoms” means that the heteroaryl group may consist of 5 ring atoms, 6 ring atoms, etc., up to and including 18 ring atoms. An N-containing “heteroaromatic” or “heteroaryl” moiety refers to an aromatic group in which at least one of the skeletal atoms of the ring is a nitrogen atom. The polycyclic heteroaryl group may be fused or non-fused. The heteroatom(s) in the heteroaryl radical is optionally oxidized. One or more nitrogen atoms, if present, are optionally quaternized. The heteroaryl is attached to the rest of the molecule through any atom of the ring(s). Examples of heteroaryls include, but are not limited to, azepinyl, acridinyl, benzimidazolyl, benzindolyl, 1,3-benzodioxolyl, benzofuranyl, benzooxazolyl, benzo[d]thiazolyl, benzothiadiazolyl, benzo[b][1,4]dioxepinyl, benzo[b][1,4]oxazinyl, 1,4-benzodioxanyl, benzonaphthofuranyl, benzoxazolyl, benzodioxolyl, benzodioxinyl, benzoxazolyl, benzopyranyl, benzopyranonyl, benzofuranyl, benzofuranonyl, benzofurazanyl, benzothiazolyl, benzothienyl (benzothiophenyl), benzothieno[3,2-d]pyrimidinyl, benzotriazolyl, benzo[4,6]imidazo[1,2-a]pyridinyl, carbazolyl, cinnolinyl, cyclopenta[d]pyrimidinyl, 6,7-dihydro-5H-cyclopenta[4,5]thieno[2,3-d]pyrimidinyl, 5,6-dihydrobenzo[h]quinazolinyl, 5,6-dihydrobenzo[h]cinnolinyl, 6,7-dihydro-5H-benzo[6,7]cyclohepta[1,2-c]pyridazinyl, dibenzofuranyl, dibenzothiophenyl, furanyl, furazanyl, furanonyl, furo[3,2-c]pyridinyl, 5,6,7,8,9,10-hexahydrocycloocta[d]pyrimidinyl, 5,6,7,8,9,10-hexahydrocycloocta[d]pyridazinyl, 5,6,7,8,9,10-hexahydrocycloocta[d]pyridinyl, isothiazolyl, imidazolyl, indazolyl, indolyl, indazolyl, isoindolyl, indolinyl, isoindolinyl, isoquinolyl, indolizinyl, isoxazolyl, 5,8-methano-5,6,7,8-tetrahydroquinazolinyl, naphthyridinyl, 1,6-naphthyridinonyl, oxadiazolyl, 2-oxoazepinyl, oxazolyl, oxiranyl, 5,6,6a,7,8,9,10,10a-octahydrobenzo[h]quinazolinyl, 1-phenyl-1H-pyrrolyl, phenazinyl, phenothiazinyl, phenoxazinyl, phthalazinyl, pteridinyl, purinyl, pyranyl, pyrrolyl, pyrazolyl, pyrazolo[3,4-d]pyrimidinyl, pyridinyl, pyrido[3,2-d]pyrimidinyl, pyrido[3,4-d]pyrimidinyl, pyrazinyl, pyrimidinyl, pyridazinyl, pyrrolyl, quinazolinyl, quinoxalinyl, quinolinyl, isoquinolinyl, tetrahydroquinolinyl, 5,6,7,8-tetrahydroquinazolinyl, 5,6,7,8-tetrahydrobenzo[4,5]thieno[2,3-d]pyrimidinyl, 6,7,8,9-tetrahydro-5H-cyclohepta[4,5]thieno[2,3-d]pyrimidinyl, 5,6,7,8-tetrahydropyrido[4,5-c]pyridazinyl, thiazolyl, thiadiazolyl, thiapyranyl, triazolyl, tetrazolyl, triazinyl, thieno[2,3-d]pyrimidinyl, thieno[3,2-d]pyrimidinyl, thieno[2,3-c]pyridinyl, and thiophenyl (i.e. thienyl). Unless stated otherwise specifically in the specification, a heteraryl moiety is optionally substituted by one or more substituents which are independently: hydroxyl, carboxaldehyde, amine, C₁-C₁₀ alkyl, C₂-C₁₀ alkynyl, C₂-C₁₀ alkenyl, carboxyl, carbohydrate, ester, acyloxy, nitro, halogen, C₁-C₁₀ aliphatic acyl, C₆-C₁₀ aromatic acyl, C₆-C₁₀ aralkyl acyl, C₆-C₁₀ alkylaryl acyl, alkoxy, alkyl, phosphate, aryl, heteroaryl, C₃-C₁₀heterocyclic, C₃-C₁₀cycloalkyl, CN, —OR^{a}—SR^{a}, —OC(O)—R^{a}, —N(R^{a})₂, —C(O)R^{a}, —C(O)OR^{a}, —C(O)N(R^{a})₂, —N(R^{a})C(O)OR^{a}, —N(R^{a})C(O)R^{a}, —N(R^{a})S(O)ₜR^{a} (where t is 1 or 2), —S(O)ₜOR^{a} (where t is 1 or 2), —S(O)ₜN(R^{a})₂ (where t is 1 or 2), —OPO₃WY (where W and Y are hydrogen, methyl, ethyl, alkyl, carbohydrate, lithium, sodium or potassium) or —OPO₃Z (where Z is calcium, magnesium or iron) where each R^{a} is independently hydrogen, alkyl, fluoroalkyl, carbocyclyl, carbocyclylalkyl, aryl, aralkyl, heterocyclyl, heterocyclylalkyl, heteroaryl or heteroarylalkyl.

“Heterocyclyl” refers to a stable 3- to 18-membered non-aromatic ring (e.g., C₃-C₁₈ heterocyclyl) radical that comprises two to twelve carbon atoms and from one to six heteroatoms selected from nitrogen, oxygen and sulfur. Whenever it appears herein, a numerical range such as “3 to 18” refers to each integer in the given range; e.g., “3 to 18 ring atoms” means that the heteroaryl group may consist of 3 ring atoms, 4 ring atoms, etc., up to and including 18 ring atoms. In some embodiments, it is a C₅-C₁₀ heterocyclyl. In some embodiments, it is a C₄-C₁₀ heterocyclyl. In some embodiments, it is a C₃-C₁₀heterocyclyl. Unless stated otherwise specifically in the specification, the heterocyclyl radical is a monocyclic, bicyclic, tricyclic or tetracyclic ring system, which may include fused or bridged ring systems. The heteroatoms in the heterocyclyl radical may be optionally oxidized. One or more nitrogen atoms, if present, are optionally quaternized. The heterocyclyl radical is partially or fully saturated. The heterocyclyl may be attached to the rest of the molecule through any atom of the ring(s). Examples of such heterocyclyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. Unless stated otherwise specifically in the specification, a heterocyclyl moiety is optionally substituted by one or more substituents which are independently: hydroxyl, carboxaldehyde, amine, C₁-C₁₀ alkyl, C₂-C₁₀ alkynyl, C₂-C₁₀ alkenyl, carboxyl, carbohydrate, ester, acyloxy, nitro, halogen, C₁-C₁₀ aliphatic acyl, C₆-C₁₀ aromatic acyl, C₆-C₁₀ aralkyl acyl, C₆-C₁₀ alkylaryl acyl, alkoxy, alkyl, phosphate, aryl, heteroaryl, C₃-C₁₀heterocyclic, C₃-C₁₀cycloalkyl, —CN, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —N(R^{a})C(O)R^{a}, —N(R^{a})S(O)ₜR^{a} (where t is 1 or 2), —S(O)ₜOR^{a} (where t is 1 or 2), —S(O)ₜN(R^{a})₂ (where t is 1 or 2), —OPO₃WY (where W and Y are hydrogen, methyl, ethyl, alkyl, carbohydrate, lithium, sodium or potassium) or —OPO₃Z (where Z is calcium, magnesium or iron) where each R^{a} is independently hydrogen, alkyl, fluoroalkyl, carbocyclyl, carbocyclylalkyl, aryl, aralkyl, heterocyclyl, heterocyclylalkyl, heteroaryl or heteroarylalkyl.

“Heteroalicyclic” refers to a cycloalkyl radical that includes at least one heteroatom selected from nitrogen, oxygen and sulfur. The radicals may be fused with an aryl or heteroaryl. The term heteroalicyclic also includes all ring forms of the carbohydrates, including but not limited to the monosaccharides, the disaccharides and the oligosaccharides. Unless stated otherwise specifically in the specification, a heteroalicyclic group is optionally substituted by one or more substituents which independently are: halo, cyano, nitro, oxo, thioxo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —N(R^{a})C(O)R^{a}, —N(R^{a})S(O)ₜR^{a} (where t is 1 or 2), —S(O)ₜOR^{a} (where t is 1 or 2), —S(O)ₜN(R^{a})₂ (where t is 1 or 2), —OPO₃WY (where W and Y are hydrogen, methyl, ethyl, alkyl, carbohydrate, lithium, sodium or potassium) or —OPO₃Z (where Z is calcium, magnesium or iron) where each R^{a} is independently hydrogen, alkyl, fluoroalkyl, carbocyclyl, carbocyclylalkyl, aryl, aralkyl, heterocyclyl, heterocyclylalkyl, heteroaryl or heteroarylalkyl.

“Imino” refers to the ═N—H radical.

“Isocyanato” refers to a —NCO radical.

“Isothiocyanato” refers to a —NCS radical.

“Mercaptyl” refers to a (alkyl)S or (H)S radical.

“Moiety” refers to a specific segment or functional group of a molecule. Chemical moieties are often recognized chemical entities embedded in or appended to a molecule.

“Nitro” refers to the NO₂ radical.

“Oxa” refers to the —O— radical.

“Oxo” refers to the ═O radical.

“Sulfinyl” refers to a —S(═O)—R radical, where R is selected from the group consisting of alkyl, cycloalkyl, aryl, heteroaryl (bonded through a ring carbon) and heteroalicyclic (bonded through a ring carbon)

“Sulfonyl” refers to a —S(═O)₂—R radical, where R is selected from the group consisting of alkyl, cycloalkyl, aryl, heteroaryl (bonded through a ring carbon) and heteroalicyclic (bonded through a ring carbon).

“Sulfonamidyl” refers to a —S(═O)₂—NRR radical, where each R is selected independently from the group consisting of hydrogen, alkyl, cycloalkyl, aryl, heteroaryl (bonded through a ring carbon) and heteroalicyclic (bonded through a ring carbon).

“Sulfoxyl” refers to a —S(═O)₂OH radical.

“Sulfonate” refers to a —S(═O)₂—OR radical, where R is selected from the group consisting of alkyl, cycloalkyl, aryl, heteroaryl (bonded through a ring carbon) and heteroalicyclic (bonded through a ring carbon).

“Thiocyanato” refers to a —CNS radical.

“Thioxo” refers to the ═S radical.

“Substituted” means that the referenced group may be substituted with one or more additional group(s) individually and independently selected from acyl, alkyl, alkylaryl, cycloalkyl, aralkyl, aryl, carbohydrate, heteroaryl, heterocyclic, hydroxy, alkoxy, aryloxy, mercapto, alkylthio, arylthio, cyano, halo, carbonyl, ester, thiocarbonyl, isocyanato, thiocyanato, isothiocyanato, nitro, perhaloalkyl, perfluoroalkyl, phosphate, silyl, sulfinyl, sulfonyl, sulfonamidyl, sulfoxyl, sulfonate, and amino, including mono- and di-substituted amino groups, and the protected derivatives thereof. The substituents themselves may be substituted, for example, a cycloakyl substituent may have a halide substituted at one or more ring carbons, and the like. The protecting groups that may form the protective derivatives of the above substituents are known to those of skill in the art and may be found in references such as Greene and Wuts, above.

The compounds presented herein may possess one or more chiral centers and each center may exist in the R or S configuration. The compounds presented herein include all diastereomeric, enantiomeric, and epimeric forms as well as the appropriate mixtures thereof. Stereoisomers may be obtained, if desired, by methods known in the art as, for example, the separation of stereoisomers by chiral chromatographic columns.

The methods and formulations described herein include the use of N-oxides, crystalline forms (also known as polymorphs), or pharmaceutically acceptable salts of compounds having the structure of Formula (I), as well as active metabolites of these compounds having the same type of activity. In addition, the compounds described herein can exist in unsolvated as well as solvated forms with pharmaceutically acceptable solvents such as water, ethanol, and the like. The solvated forms of the compounds presented herein are also considered to be disclosed herein.

Pyrone analogs of Formula I and their pharmaceutically/veterinarily acceptable salt or esters are provided in this invention,

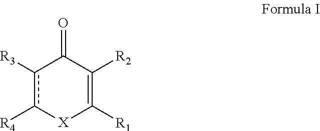

wherein X is O, S, or NR′, wherein R′ is hydrogen, C₁-C₁₀ alkyl, C₂-C₁₀ alkynyl, C₂-C₁₀ alkenyl, C₁-C₁₀ aliphatic acyl, C₆-C₁₀ aromatic acyl, C₆-C₁₀ aralkyl acyl, C₆-C₁₀ alkylaryl acyl, aryl, C₃-C₁₀ heterocyclyl, heteroaryl, or C₃-C₁₀cycloalkyl;

R₁, and R₂ are independently hydrogen, hydroxyl, C₁-C₁₀ alkyl, C₂-C₁₀ alkynyl, C₂-C₁₀ alkenyl, carboxyl, carbohydrate, ester, acyloxy, nitro, halogen, C₁-C₁₀ aliphatic acyl, C₆-C₁₀ aromatic acyl, C₆-C₁₀ aralkyl acyl, C₆-C₁₀ alkylaryl acyl, alkoxy, amine, aryl, C₄-C₁₀heterocyclyl, heteroaryl, C₃-C₁₀cycloalkyl, —OPO₃WY, —OCH₂PO₄WY, —OCH₂PO₄Z or —OPO₃Z;

R₃ and R₄ are independently hydrogen, hydroxyl, C₁-C₁₀ alkyl, C₂-C₁₀ alkynyl, C₂-C₁₀ alkenyl, carboxyl, Carbohydrate, ester, acyloxy, nitro, halogen, C₁-C₁₀ aliphatic acyl, C₆-C₁₀ aromatic acyl C₆-C₁₀ aralkyl acyl, C₆-C₁₀ alkylaryl acyl, alkoxy, amine, aryl, C₄-C₁₀heterocyclyl, heteroaryl, C₃-C₁₀cycloalkyl, —OPO₃WY, —OCH₂PO₄WY, —OCH₂PO₄Z or —OPO₃Z;

or R₃ and R₄ are taken together to form a C₅-C₁₀heterocyclyl, C₅-C₁₀cycloalkyl, aryl, or heteroaryl;

W and Y are independently hydrogen, methyl, ethyl, alkyl, carbohydrate, or a cation, and Z is a multivalent cation; and

wherein the compound of Formula I is not the compound of Formula A:

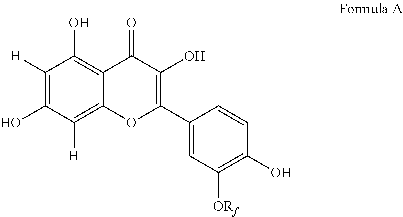

wherein R_{f} is hydrogen; and

wherein the compound of Formula I is not one of the following compounds:

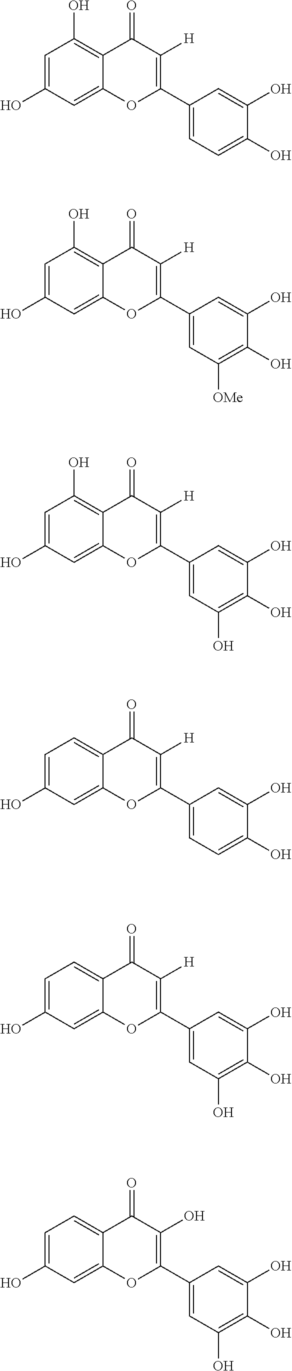

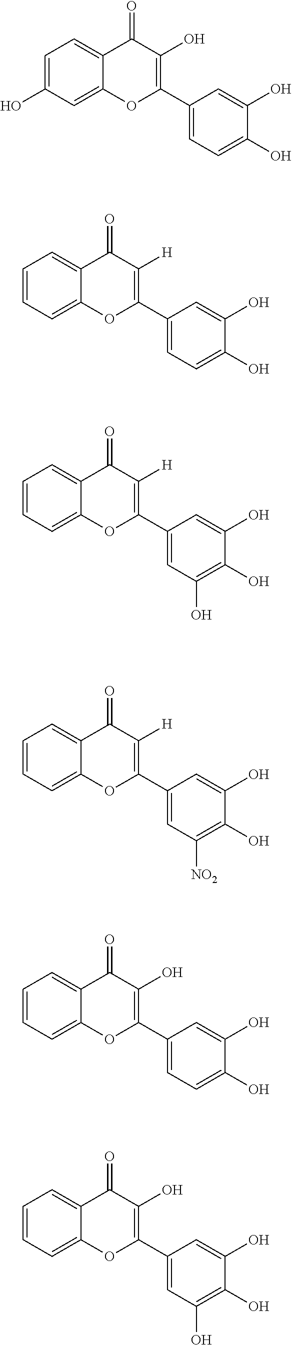

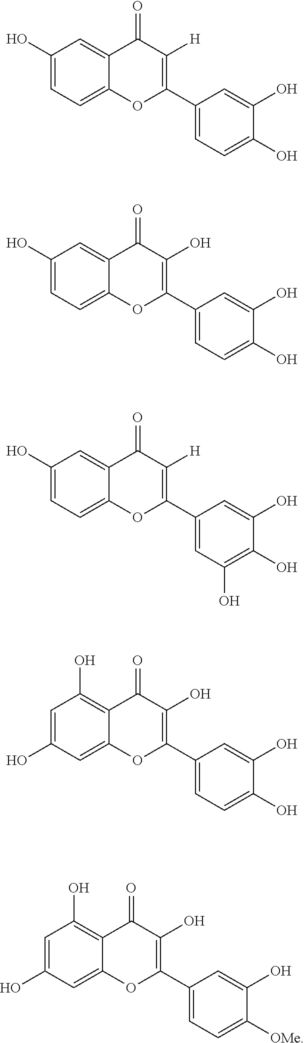

In some embodiments, the compound of Formula I is not the compound of Formula A wherein R_{f} is PO₃K₂.

In some embodiments, X is O.

In other embodiments, X is S.

In yet other embodiments, X is NR′.

In some embodiments, R′ is hydrogen. In some embodiments, R′ is unsubstituted C₁-C₁₀ alkyl. In some embodiments, R′ is substituted C₁-C₁₀ alkyl. In some embodiments, R′ is unsubstituted C₂-C₁₀ alkynyl. In some embodiments, R′ is substituted C₂-C₁₀ alkynyl. In some embodiments, R′ is unsubstituted C₂-C₁₀ alkenyl. In some embodiments, R′ is substituted C₂-C₁₀ alkenyl. In some embodiments, R′ is unsubstituted C₁-C₁₀ aliphatic acyl. In some embodiments, R′ is substituted C₁-C₁₀ aliphatic acyl. In some embodiments, R′ is unsubstituted C₆-C₁₀ aromatic acyl. In some embodiments, R′ is substituted C₆-C₁₀ aromatic acyl. In some embodiments, R′ is unsubstituted C₆-C₁₀ aralkyl acyl. In some embodiments, R′ is substituted C₆-C₁₀ aralkyl acyl. In some embodiments, R′ is unsubstituted C₆-C₁₀ alkylaryl acyl. In some embodiments, R′ is substituted C₆-C₁₀ alkylaryl acyl. In some embodiments, R′ is unsubstituted aryl. In some embodiments, R′ is substituted aryl. In some embodiments, R′ is unsubstituted C₃-C₁₀ heterocyclyl. In some embodiments, R′ is substituted C₃-C₁₀ heterocyclyl. In some embodiments, R′ is unsubstituted heteroaryl. In some embodiments, R′ is substituted heteroaryl. In some embodiments, R′ is unsubstituted C₃-C₁₀Cycloalkyl. In some embodiments, R′ is substituted C₃-C₁₀cycloalkyl.

In some embodiments, R₁ is hydrogen. In some embodiments, R₁ is hydroxyl. In some embodiments, R₁ is optionally substituted C₁-C₁₀ alkyl. In some embodiments, R₁ is unsubstituted C₁-C₁₀ alkyl. In some embodiments, R₁ is substituted C₁-C₁₀ alkyl. In some embodiments, R₁ is unsubstituted C₁-C₁₀ alkyl. In some other embodiments, R₁ is substituted C₁-C₁₀ alkyl. In some embodiments, R₁ is unsubstituted C₂-C₁₀ alkynyl. In some embodiments, R₁ is substituted C₂-C₁₀ alkynyl. In some embodiments, R₁ is unsubstituted C₂-C₁₀ alkenyl. In some embodiments, R₁ is substituted C₂-C₁₀ alkenyl. In some embodiments, R₁ is carboxyl. In some embodiments, R₁ is unsubstituted carbohydrate. In some embodiments, R₁ is substituted carbohydrate. In some embodiments, R₁ is unsubstituted ester. In some embodiments, R₁ is substituted ester. In some embodiments, R₁ is unsubstituted acyloxy. In some embodiments, R₁ is substituted acyloxy. In some embodiments, R₁ is nitro. In some embodiments, R₁ is halogen. In some embodiments, R₁ is unsubstituted C₁-C₁₀ aliphatic acyl. In some embodiments, R₁ is substituted C₁-C₁₀ aliphatic acyl. In some embodiments, R₁ is unsubstituted C₆-C₁₀ aromatic acyl. In some embodiments, R₁ is substituted C₆-C₁₀ aromatic acyl. In some embodiments, R₁ is unsubstituted C₆-C₁₀ aralkyl acyl. In some embodiments, R₁ is substituted C₆-C₁₀ aralkyl acyl. In some embodiments, R₁ is unsubstituted C₆-C₁₀ alkylaryl acyl. In some embodiments, R₁ is substituted C₆-C₁₀ alkylaryl acyl. In some embodiments, R₁ is unsubstituted alkoxy. In some embodiments, R₁ is substituted alkoxy. In some embodiments, R₁ is unsubstituted amine. In some embodiments, R₁ is substituted amine. In some embodiments, R₁ is unsubstituted aryl. In some embodiments, R₁ is substituted aryl. In some embodiments, R₁ is unsubstituted C₄-C₁₀heterocyclyl. In some embodiments, R₁ is substituted C₄-C₁₀heterocyclyl. In some embodiments, R₁ is unsubstituted heteroaryl. In some embodiments, R₁ is substituted heteroaryl. In some embodiments, R₁ is unsubstituted C₃-C₁₀cycloalkyl. In some embodiments, R₁ is substituted C₃-C₁₀cycloalkyl. In some embodiments, R₁ is —OPO₃WY. In some embodiments, R₁ is —OCH₂PO₄WY. In some embodiments, R₁ is —OCH₂PO₄Z. In some embodiments, R₁ is —OPO₃Z.

In some embodiments, when R₁ is aryl, it is monocyclic. In some embodiments, when R₁ is aryl, it is bicyclic. In some embodiments, when R₁ is heteroaryl, it is monocyclic. In some embodiments, when R₁ is heteroaryl, it is bicyclic.

In some embodiments, R₂ is hydrogen. In some embodiments, R₂ is hydroxyl. In some embodiments, R₂ is optionally substituted C₁-C₁₀ alkyl. In some embodiments, R₂ is unsubstituted C₁-C₁₀ alkyl. In some embodiments, R₂ is substituted C₁-C₁₀ alkyl. In some embodiments, R₂ is unsubstituted C₁-C₁₀ alkyl. In some other embodiments, R₂ is substituted C₁-C₁₀ alkyl. In some embodiments, R₂ is unsubstituted C₂-C₁₀ alkynyl. In some embodiments, R₂ is substituted C₂-C₁₀ alkynyl. In some embodiments, R₂ is unsubstituted C₂-C₁₀ alkenyl. In some embodiments, R₂ is substituted C₂-C₁₀ alkenyl. In some embodiments, R₂ is carboxyl. In some embodiments, R₂ is unsubstituted carbohydrate. In some embodiments, R₂ is substituted carbohydrate. In some embodiments, R₂ is unsubstituted ester. In some embodiments, R₂ is substituted ester. In some embodiments, R₂ is unsubstituted acyloxy. In some embodiments, R₂ is substituted acyloxy. In some embodiments, R₂ is nitro. In some embodiments, R₂ is halogen. In some embodiments, R₂ is unsubstituted C₁-C₁₀ aliphatic acyl. In some embodiments, R₂ is substituted C₁-C₁₀ aliphatic acyl. In some embodiments, R₂ is unsubstituted C₆-C₁₀ aromatic acyl. In some embodiments, R₂ is substituted C₆-C₁₀ aromatic acyl. In some embodiments, R₂ is unsubstituted C₆-C₁₀ aralkyl acyl. In some embodiments, R₂ is substituted C₆-C₁₀ aralkyl acyl. In some embodiments, R₂ is unsubstituted C₆-C₁₀ alkylaryl acyl. In some embodiments, R₂ is substituted C₆-C₁₀ alkylaryl acyl. In some embodiments, R₂ is unsubstituted alkoxy. In some embodiments, R₂ is substituted alkoxy. In some embodiments, R₂ is unsubstituted amine. In some embodiments, R₂ is substituted amine. In some embodiments, R₂ is unsubstituted aryl. In some embodiments, R₂ is substituted aryl. In some embodiments, R₂ is unsubstituted C₄-C₁₀heterocyclyl. In some embodiments, R₂ is substituted C₄-C₁₀heterocyclyl. In some embodiments, R₂ is unsubstituted heteroaryl. In some embodiments, R₂ is substituted heteroaryl. In some embodiments, R₂ is unsubstituted C₃-C₁₀cycloalkyl. In some embodiments, R₂ is substituted C₃-C₁₀cycloalkyl. In some embodiments, R₂ is —OPO₃WY. In some embodiments, R₂ is —OCH₂PO₄WY. In some embodiments, R₂ is —OCH₂PO₄Z. In some embodiments, R₂ is —OPO₃Z.

In some embodiments, R₃ is hydrogen. In some embodiments, R₃ is hydroxyl. In some embodiments, R₃ is optionally substituted C₁-C₁₀ alkyl. In some embodiments, R₃ is unsubstituted C₁-C₁₀ alkyl. In some embodiments, R₃ is substituted C₁-C₁₀ alkyl. In some embodiments, R₃ is unsubstituted C₁-C₁₀ alkyl. In some other embodiments, R₃ is substituted C₁-C₁₀ alkyl. In some embodiments, R₃ is unsubstituted C₂-C₁₀ alkynyl. In some embodiments, R₃ is substituted C₂-C₁₀ alkynyl. In some embodiments, R₃ is unsubstituted C₂-C₁₀ alkenyl. In some embodiments, R₃ is substituted C₂-C₁₀ alkenyl. In some embodiments, R₃ is carboxyl. In some embodiments, R₃ is unsubstituted carbohydrate. In some embodiments, R₃ is substituted carbohydrate. In some embodiments, R₃ is unsubstituted ester. In some embodiments, R₃ is substituted ester. In some embodiments, R₃ is unsubstituted acyloxy. In some embodiments, R₃ is substituted acyloxy. In some embodiments, R₃ is nitro. In some embodiments, R₃ is halogen. In some embodiments, R₃ is unsubstituted C₁-C₁₀ aliphatic acyl. In some embodiments, R₃ is substituted C₁-C₁₀ aliphatic acyl. In some embodiments, R₃ is unsubstituted C₆-C₁₀ aromatic acyl. In some embodiments, R₃ is substituted C₆-C₁₀ aromatic acyl. In some embodiments, R₃ is unsubstituted C₆-C₁₀ aralkyl acyl. In some embodiments, R₃ is substituted C₆-C₁₀ aralkyl acyl. In some embodiments, R₃ is unsubstituted C₆-C₁₀ alkylaryl acyl. In some embodiments, R₃ is substituted C₆-C₁₀ alkylaryl acyl. In some embodiments, R₃ is unsubstituted alkoxy. In some embodiments, R₃ is substituted alkoxy. In some embodiments, R₃ is unsubstituted amine. In some embodiments, R₃ is substituted amine. In some embodiments, R₃ is unsubstituted aryl. In some embodiments, R₃ is substituted aryl. In some embodiments, R₃ is unsubstituted C₄-C₁₀heterocyclyl. In some embodiments, R₃ is substituted C₄-C₁₀heterocyclyl. In some embodiments, R₃ is unsubstituted heteroaryl. In some embodiments, R₃ is substituted heteroaryl. In some embodiments, R₃ is unsubstituted C₃-C₁₀cycloalkyl. In some embodiments, R₃ is substituted C₃-C₁₀cycloalkyl. In some embodiments, R₃ is —OPO₃WY. In some embodiments, R₃ is —OCH₂PO₄WY. In some embodiments, R₃ is —OCH₂PO₄Z. In some embodiments, R₃ is —OPO₃Z.

In some embodiments, R₄ is hydrogen. In some embodiments, R₄ is hydroxyl. In some embodiments, R₄ is optionally substituted C₁-C₁₀ alkyl. In some embodiments, R₄ is unsubstituted C₁-C₁₀ alkyl. In some embodiments, R₄ is substituted C₁-C₁₀ alkyl. In some embodiments, R₄ is unsubstituted C₁-C₁₀ alkyl. In some other embodiments, R₄ is substituted C₁-C₁₀ alkyl. In some embodiments, R₄ is unsubstituted C₂-C₁₀ alkynyl. In some embodiments, R₄ is substituted C₂-C₁₀ alkynyl. In some embodiments, R₄ is unsubstituted C₂-C₁₀ alkenyl. In some embodiments, R₄ is substituted C₂-C₁₀ alkenyl. In some embodiments, R₄ is carboxyl. In some embodiments, R₄ is unsubstituted carbohydrate. In some embodiments, R₄ is substituted carbohydrate. In some embodiments, R₄ is unsubstituted ester. In some embodiments, R₄ is substituted ester. In some embodiments, R₄ is unsubstituted acyloxy. In some embodiments, R₄ is substituted acyloxy. In some embodiments, R₄ is nitro. In some embodiments, R₄ is halogen. In some embodiments, R₄ is unsubstituted C₁-C₁₀ aliphatic acyl. In some embodiments, R₄ is substituted C₁-C₁₀ aliphatic acyl. In some embodiments, R₄ is unsubstituted C₆-C₁₀ aromatic acyl. In some embodiments, R₄ is substituted C₆-C₁₀ aromatic acyl. In some embodiments, R₄ is unsubstituted C₆-C₁₀ aralkyl acyl. In some embodiments, R₄ is substituted C₆-C₁₀ aralkyl acyl. In some embodiments, R₄ is unsubstituted C₆-C₁₀ alkylaryl acyl. In some embodiments, R₄ is substituted C₆-C₁₀ alkylaryl acyl. In some embodiments, R₄ is unsubstituted alkoxy. In some embodiments, R₄ is substituted alkoxy. In some embodiments, R₄ is unsubstituted amine. In some embodiments, R₄ is substituted amine. In some embodiments, R₄ is unsubstituted aryl. In some embodiments, R₄ is substituted aryl. In some embodiments, R₄ is unsubstituted C₄-C₁₀heterocyclyl. In some embodiments, R₄ is substituted C₄-C₁₀heterocyclyl. In some embodiments, R₄ is unsubstituted heteroaryl. In some embodiments, R₄ is substituted heteroaryl. In some embodiments, R₄ is unsubstituted C₃-C₁₀cycloalkyl. In some embodiments, R₄ is substituted C₃-C₁₀cycloalkyl. In some embodiments, R₄ is —OPO₃WY. In some embodiments, R₄ is —OCH₂PO₄WY. In some embodiments, R₄ is —OCH₂PO₄Z. In some embodiments, R₄ is —OPO₃Z.

In some embodiments, R₃ and R₄ are taken together to form an unsubstituted C₅-C₁₀heterocyclyl. In other embodiments, R₃ and R₄ are taken together to form a substituted C₅-C₁₀heterocyclyl. In some embodiments, R₃ and R₄ are taken together to form an unsubstituted C₅-C₁₀cycloalkyl. In some embodiments, R₃ and R₄ are taken together to form a substituted C₅-C₁₀cycloalkyl. In some embodiments, R₃ and R₄ are taken together to form an unsubstituted aryl. In some embodiments, R₃ and R₄ are taken together to form a substituted aryl. In some embodiments, R₃ and R₄ are taken together to form an unsubstituted heteroaryl. In some embodiments, R₃ and R₄ are taken together to form a substituted heteroaryl.

In various embodiments, W is hydrogen. In various embodiments, W is unsubstituted methyl. In various embodiments, W is substituted methyl. In various embodiments, W is unsubstituted ethyl. In various embodiments, W is substituted ethyl. In various embodiments, W is unsubstituted alkyl. In various embodiments, W is substituted alkyl. In various embodiments, W is unsubstituted carbohydrate. In various embodiments, W is substituted carbohydrate. In various embodiments, W is potassium. In various embodiments, W is sodium. In various embodiments, W is lithium.

In various embodiments, Y is hydrogen. In various embodiments, Y is unsubstituted methyl. In various embodiments, Y is substituted methyl. In various embodiments, Y is unsubstituted ethyl. In various embodiments, Y is substituted ethyl. In various embodiments, Y is unsubstituted alkyl. In various embodiments, Y is substituted alkyl. In various embodiments, Y is unsubstituted carbohydrate. In various embodiments, Y is substituted carbohydrate. In various embodiments, Y is potassium. In various embodiments, Y is sodium. In various embodiments, Y is lithium.

In various embodiments, Z is calcium. In various embodiments, Z is magnesium. In various embodiments, Z is iron.

The 2,3 bond may be saturated or unsaturated in the compounds of Formula I.

In some embodiments of the invention, the pyrone analog is of Formula II and its pharmaceutically/veterinarily acceptable salts thereof:

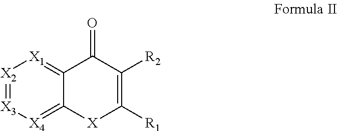

wherein X is O, S, or NR′, wherein R′ is hydrogen, C₁-C₁₀ alkyl, C₂-C₁₀ alkynyl, C₂-C₁₀ alkenyl, C₁-C₁₀ aliphatic acyl, C₆-C₁₀ aromatic acyl, C₆-C₁₀ aralkyl acyl, C₆-C₁₀ alkylaryl acyl, aryl, C₃-C₁₀ heterocyclyl, heteroaryl, or C₃-C₁₀cycloalkyl;

R₁, and R₂ are independently hydrogen, hydroxyl, C₁-C₁₀ alkyl, C₂-C₁₀ alkynyl, C₂-C₁₀ alkenyl, carboxyl, carbohydrate, ester, acyloxy, nitro, halogen, C₁-C₁₀ aliphatic acyl, C₆-C₁₀ aromatic acyl, C₆-C₁₀ aralkyl acyl, C₆-C₁₀ alkylaryl acyl, alkoxy, amine, aryl, C₄-C₁₀heterocyclyl, heteroaryl, C₃-C₁₀cycloalkyl, —OPO₃WY, —OCH₂PO₄WY, —OCH₂PO₄Z or —OPO₃Z;

X₁, X₂, X₃, and X₄ are independently CR₅, O, S, or N;

each instance of R₅ is independently hydrogen, hydroxyl, carboxaldehyde, amino, C₁-C₁₀ alkyl, C₂-C₁₀ alkynyl, C₂-C₁₀ alkenyl, carboxyl, Carbohydrate, ester, acyloxy, nitro, halogen, C₁-C₁₀ aliphatic acyl, C₆-C₁₀ aromatic acyl, C₆-C₁₀ aralkyl acyl, C₆-C₁₀alkylaryl acyl, alkoxy, amine, aryl, C₃-C₁₀heterocyclyl, heteroaryl, C₃-C₁₀cycloalkyl, —OPO₃WY, —OCH₂PO₄WY, —OCH₂PO₄Z or —OPO₃Z;

W and Y are independently hydrogen, methyl, ethyl, alkyl carbohydrate, or a cation, and Z is a multivalent cation; and

wherein the compound of Formula II is not the compound of Formula A:

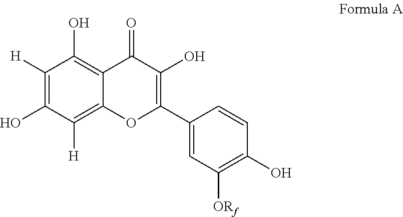

wherein R_{f} is hydrogen; and

wherein the compound of Formula II is not one of the following compounds:

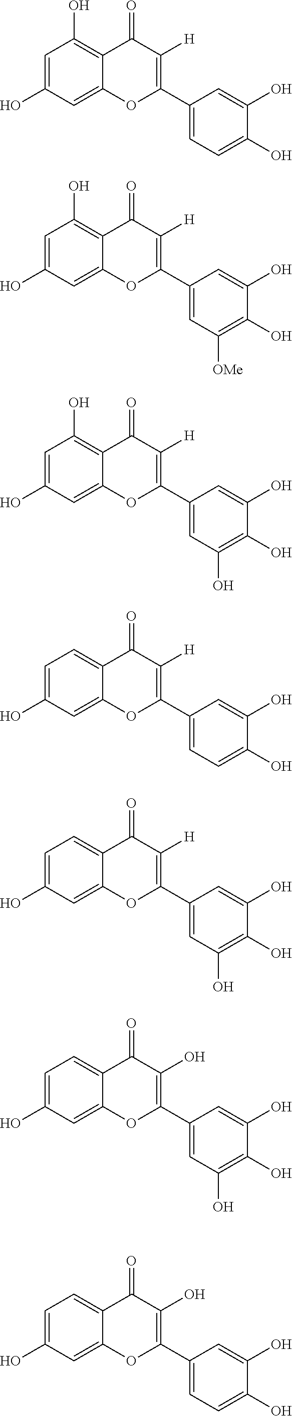

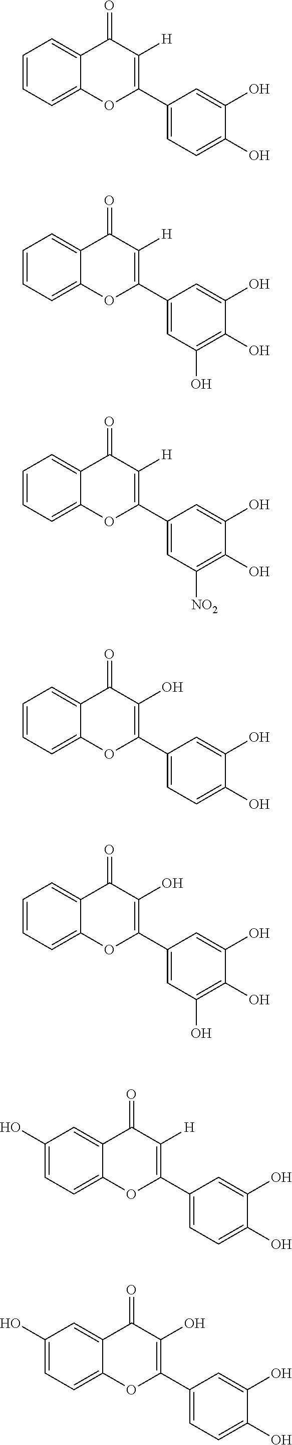

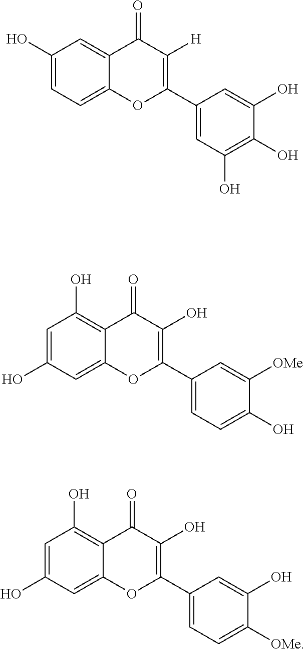

In some embodiments, the compound of Formula II is not the compound of Formula A wherein R_{f} is PO₃K₂.

In some embodiments, X is O.

In other embodiments, X is S.

In yet other embodiments, X is NR′.

In some embodiments, R′ is hydrogen. In some embodiments, R′ is unsubstituted C₁-C₁₀ alkyl. In some embodiments, R′ is substituted C₁-C₁₀ alkyl. In some embodiments, R′ is unsubstituted C₂-C₁₀ alkynyl. In some embodiments, R′ is substituted C₂-C₁₀ alkynyl. In some embodiments, R′ is unsubstituted C₂-C₁₀ alkenyl. In some embodiments, R′ is substituted C₂-C₁₀ alkenyl. In some embodiments, R′ is unsubstituted C₂-C₁₀ alkenyl. In some embodiments, R′ is substituted C₂-C₁₀ alkenyl. In some embodiments, R′ is unsubstituted C₁-C₁₀ aliphatic acyl. In some embodiments, R′ is substituted C₁-C₁₀ aliphatic acyl. In some embodiments, R′ is unsubstituted C₆-C₁₀ aromatic acyl. In some embodiments, R′ is substituted C₆-C₁₀ aromatic acyl. In some embodiments, R′ is unsubstituted C₆-C₁₀ aralkyl acyl. In some embodiments, R′ is substituted C₆-C₁₀ aralkyl acyl. In some embodiments, R′ is unsubstituted C₆-C₁₀ alkylaryl acyl. In some embodiments, R′ is substituted C₆-C₁₀ alkylaryl acyl. In some embodiments, R′ is unsubstituted aryl. In some embodiments, R′ is substituted aryl. In some embodiments, R′ is unsubstituted C₃-C₁₀ heterocyclyl. In some embodiments, R′ is substituted C₃-C₁₀ heterocyclyl. In some embodiments, R′ is unsubstituted heteroaryl. In some embodiments, R′ is substituted heteroaryl. In some embodiments, R′ is unsubstituted C₃-C₁₀Cycloalkyl. In some embodiments, R′ is substituted C₃-C₁₀cycloalkyl.

In some embodiments, X₁ is CR₅.

In other embodiments, X₁ is O.

In yet other embodiments, X₁ is S.

In further embodiments, X₁ is N.

In some embodiments, X₂ is CR₅.

In other embodiments, X₂ is O.

In yet other embodiments, X₂ is S.

In further embodiments, X₂ is N.

In some embodiments, X₃ is CR₅.

In other embodiments, X₃ is O.

In yet other embodiments, X₃ is S.

In further embodiments, X₃ is N.

In other embodiments, X₄ is CR₅.

In some embodiments, X₄ is O.

In yet other embodiments, X₄ is S.

In some embodiments, X₄ is N.

In some embodiments, X₁, X₂, X₃, and X₄ are CR₅.

In some embodiments, X₁ is N, and X₂, X₃, and X₄ are CR₅.

In some embodiments, X₂ is N, and X₁, X₃, and X₄ are CR₅;

In some embodiments, X₃ is N, and X₁, X₂, and X₄ are CR₅;

In some embodiments, X₄ is N, and X₁, X₂, and X₃ are CR₅;

In some embodiments, X₁ and X₃ are CR₅ and X₂ and X₄ are N.

In some embodiments, X₂ and X₄ are CR₅ and X₁ and X₃ are N.

In some embodiments, X₂ and X₃ are CR₅ and X₁ and X₄ are N.

In some embodiments, at least one of X₁, X₂, X₃ or X₄ is N.

In some embodiments, R₁ is hydrogen. In some embodiments, R₁ is hydroxyl. In some embodiments, R₁ is optionally substituted C₁-C₁₀ alkyl. In some embodiments, R₁ is unsubstituted C₁-C₁₀ alkyl. In some embodiments, R₁ is substituted C₁-C₁₀ alkyl. In some embodiments, R₁ is unsubstituted C₁-C₁₀ alkyl. In some other embodiments, R₁ is substituted C₁-C₁₀ alkyl. In some embodiments, R₁ is unsubstituted C₂-C₁₀ alkynyl. In some embodiments, R₁ is substituted C₂-C₁₀ alkynyl. In some embodiments, R₁ is unsubstituted C₂-C₁₀ alkenyl. In some embodiments, R₁ is substituted C₂-C₁₀ alkenyl. In some embodiments, R₁ is carboxyl. In some embodiments, R₁ is unsubstituted carbohydrate. In some embodiments, R₁ is substituted carbohydrate. In some embodiments, R₁ is unsubstituted ester. In some embodiments, R₁ is substituted ester. In some embodiments, R₁ is unsubstituted acyloxy. In some embodiments, R₁ is substituted acyloxy. In some embodiments, R₁ is nitro. In some embodiments, R₁ is halogen. In some embodiments, R₁ is unsubstituted C₁-C₁₀ aliphatic acyl. In some embodiments, R₁ is substituted C₁-C₁₀ aliphatic acyl. In some embodiments, R₁ is unsubstituted C₆-C₁₀ aromatic acyl. In some embodiments, R₁ is substituted C₆-C₁₀ aromatic acyl. In some embodiments, R₁ is unsubstituted C₆-C₁₀ aralkyl acyl. In some embodiments, R₁ is substituted C₆-C₁₀ aralkyl acyl. In some embodiments, R₁ is unsubstituted C₆-C₁₀ alkylaryl acyl. In some embodiments, R₁ is substituted C₆-C₁₀ alkylaryl acyl. In some embodiments, R₁ is unsubstituted alkoxy. In some embodiments, R₁ is substituted alkoxy. In some embodiments, R₁ is unsubstituted amine. In some embodiments, R₁ is substituted amine. In some embodiments, R₁ is unsubstituted aryl. In some embodiments, R₁ is substituted aryl. In some embodiments, R₁ is unsubstituted C₄-C₁₀heterocyclyl. In some embodiments, R₁ is substituted C₄-C₁₀heterocyclyl. In some embodiments, R₁ is unsubstituted heteroaryl. In some embodiments, R₁ is substituted heteroaryl. In some embodiments, R₁ is unsubstituted C₃-C₁₀cycloalkyl. In some embodiments, R₁ is substituted C₃-C₁₀cycloalkyl. In some embodiments, R₁ is —OPO₃WY. In some embodiments, R₁ is —OCH₂PO₄WY. In some embodiments, R₁ is —OCH₂PO₄Z. In some embodiments, R₁ is —OPO₃Z.

In some embodiments, R₂ is hydrogen. In some embodiments, R₂ is hydroxyl. In some embodiments, R₂ is optionally substituted C₁-C₁₀ alkyl. In some embodiments, R₂ is unsubstituted C₁-C₁₀ alkyl. In some embodiments, R₂ is substituted C₁-C₁₀ alkyl. In some embodiments, R₂ is unsubstituted C₁-C₁₀ alkyl. In some other embodiments, R₂ is substituted C₁-C₁₀ alkyl. In some embodiments, R₂ is unsubstituted C₂-C₁₀ alkynyl. In some embodiments, R₂ is substituted C₂-C₁₀ alkynyl. In some embodiments, R₂ is unsubstituted C₂-C₁₀ alkenyl. In some embodiments, R₂ is substituted C₂-C₁₀ alkenyl. In some embodiments, R₂ is carboxyl. In some embodiments, R₂ is unsubstituted carbohydrate. In some embodiments, R₂ is substituted carbohydrate. In some embodiments, R₂ is unsubstituted ester. In some embodiments, R₂ is substituted ester. In some embodiments, R₂ is unsubstituted acyloxy. In some embodiments, R₂ is substituted acyloxy. In some embodiments, R₂ is nitro. In some embodiments, R₂ is halogen. In some embodiments, R₂ is unsubstituted C₁-C₁₀ aliphatic acyl. In some embodiments, R₂ is substituted C₁-C₁₀ aliphatic acyl. In some embodiments, R₂ is unsubstituted C₆-C₁₀ aromatic acyl. In some embodiments, R₂ is substituted C₆-C₁₀ aromatic acyl. In some embodiments, R₂ is unsubstituted C₆-C₁₀ aralkyl acyl. In some embodiments, R₂ is substituted C₆-C₁₀ aralkyl acyl. In some embodiments, R₂ is unsubstituted C₆-C₁₀ alkylaryl acyl. In some embodiments, R₂ is substituted C₆-C₁₀ alkylaryl acyl. In some embodiments, R₂ is unsubstituted alkoxy. In some embodiments, R₂ is substituted alkoxy. In some embodiments, R₂ is unsubstituted amine. In some embodiments, R₂ is substituted amine. In some embodiments, R₂ is unsubstituted aryl. In some embodiments, R₂ is substituted aryl. In some embodiments, R₂ is unsubstituted C₄-C₁₀heterocyclyl. In some embodiments, R₂ is substituted C₄-C₁₀heterocyclyl. In some embodiments, R₂ is unsubstituted heteroaryl. In some embodiments, R₂ is substituted heteroaryl. In some embodiments, R₂ is unsubstituted C₃-C₁₀cycloalkyl. In some embodiments, R₂ is substituted C₃-C₁₀cycloalkyl. In some embodiments, R₂ is —OPO₃WY. In some embodiments, R₂ is —OCH₂PO₄WY. In some embodiments, R₂ is —OCH₂PO₄Z. In some embodiments, R₂ is —OPO₃Z.

In some embodiments, when R₁ is aryl, it is monocyclic. In some embodiments, when R₁ is aryl, it is bicyclic. In some embodiments, when R₁ is heteroaryl, it is monocyclic. In some embodiments, when R₁ is heteroaryl, it is bicyclic.

In various embodiments, R₁ is one of the following formulae:

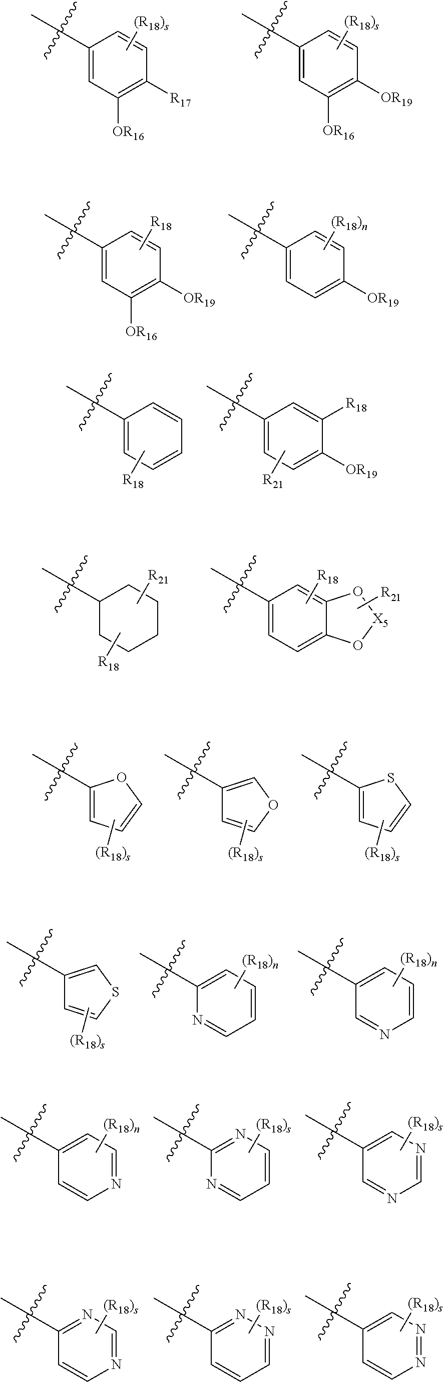

wherein R₁₆ is hydrogen, C₁-C₁₀ alkyl, C₂-C₁₀ alkynyl, C₂-C₁₀ alkenyl, carbohydrate, C₁-C₁₀ aliphatic acyl, C₆-C₁₀ aromatic acyl, C₆-C₁₀ aralkyl acyl, C₆-C₁₀ alkylaryl acyl, aryl, C₃-C₁₀ heterocyclyl, heteroaryl, C₃-C₁₀cycloalkyl, —PO₃WY, —CH₂PO₄WY, —CH₂PO₄Z or —PO₃Z;

R₁₇ is hydrogen, hydroxy, carboxaldehyde, amine, C₁-C₁₀ alkyl, C₂-C₁₀ alkynyl, C₂-C₁₀ alkenyl, carboxyl, carbohydrate, ester, acyloxy, nitro, halogen, C₁-C₁₀ aliphatic acyl, C₆-C₁₀ aromatic acyl, C₆-C₁₀ aralkyl acyl, C₆-C₁₀ alkylaryl acyl, alkoxy, aryl, C₃-C₁₀ heterocyclyl, heteroaryl, or C₃-C₁₀cycloalkyl, —OPO₃WY, —OCH₂PO₄WY, —OCH₂PO₄Z or —OPO₃Z;

each instance of R₁₈ and R₂₁ is independently hydrogen, hydroxyl, carboxaldehyde, amine, C₁-C₁₀ alkyl, C₂-C₁₀ alkynyl, C₂-C₁₀ alkenyl, carboxyl, carbohydrate, ester, acyloxy, nitro, halogen, C₁-C₁₀ aliphatic acyl, C₆-C₁₀ aromatic acyl, C₆-C₁₀ aralkyl acyl, C₆-C₁₀ alkylaryl acyl, alkoxy, alkyl, phosphate, aryl, heteroaryl, C₃-C₁₀ heterocyclic, C₃-C₁₀cycloalkyl, —OPO₃WY, —OCH₂PO₄WY, —OCH₂PO₄Z or —OPO₃Z;

R₁₉ is hydrogen, C₁-C₁₀ alkyl, C₂-C₁₀ alkynyl, C₂-C₁₀ alkenyl, carbohydrate, C₁-C₁₀ aliphatic acyl, C₆-C₁₀ aromatic acyl, C₆-C₁₀ aralkyl acyl, C₆-C₁₀ alkylaryl acyl, aryl, C₃-C₁₀heterocyclyl, heteroaryl, optionally substituted C₃-C₁₀cycloalkyl, —PO₃WY, —CH₂PO₄WY, —CH₂PO₄Z or —PO₃Z;

s is an integer of 0, 1, 2, or 3; and

n is an integer of 0, 1, 2, 3, or 4.

In some embodiments of the invention, at least one of R₁₆ and R₁₉ is —PO₃WY, —CH₂PO₄WY, —CH₂PO₄Z or —PO₃Z, or at least one of R₁₇ or R₁₈ is —OPO₃WY, —OCH₂PO₄WY, —OCH₂PO₄Z or —OPO₃Z. In some embodiments, at least one of R₁₆ and R₁₉ is —PO₃WY, —CH₂PO₄WY, —CH₂PO₄Z or —PO₃Z and at least one of R₁₇ or R₁₈ is —OPO₃WY, —OCH₂PO₄WY, —OCH₂PO₄Z or —OPO₃Z.

In some embodiments, R₁₆ is hydrogen. In some embodiments, R₁₆ is unsubstituted C₁-C₁₀ alkyl. In some embodiments, R₁₆ is substituted C₁-C₁₀ alkyl. In some embodiments, R₁₆ is unsubstituted C₂-C₁₀ alkynyl. In some embodiments, R₁₆ is substituted C₂-C₁₀ alkynyl. In some embodiments, R₁₆ is unsubstituted C₂-C₁₀ alkenyl. In some embodiments, R₁₆ is substituted C₂-C₁₀ alkenyl. In some embodiments, R₁₆ is unsubstituted carbohydrate 1. In some embodiments, R₁₆ is substituted carbohydrate. In some embodiments, R₁₆ is unsubstituted C₁-C₁₀ aliphatic acyl. In some embodiments, R₁₆ is substituted C₁-C₁₀ aliphatic acyl. In some embodiments, R₁₆ is unsubstituted C₆-C₁₀ aromatic acyl. In some embodiments, R₁₆ is substituted C₆-C₁₀ aromatic acyl. In some embodiments, R₁₆ is unsubstituted C₆-C₁₀ aralkyl acyl. In some embodiments, R₁₆ is substituted C₆-C₁₀ aralkyl acyl. In some embodiments, R₁₆ is unsubstituted C₆-C₁₀ alkylaryl acyl. In some embodiments, R₁₆ is substituted C₆-C₁₀ alkylaryl acyl. In some embodiments, R₁₆ is unsubstituted aryl. In some embodiments, R₁₆ is substituted aryl. In some embodiments, R₁₆ is unsubstituted C₃-C₁₀heterocyclyl. In some embodiments, R₁₆ is substituted C₃-C₁₀ heterocyclyl. In some embodiments, R₁₆ is unsubstituted heteroaryl. In some embodiments, R₁₆ is substituted heteroaryl. In some embodiments, R₁₆ is unsubstituted C₃-C₁₀cycloalkyl. In some embodiments, R₁₆ is substituted C₃-C₁₀cycloalkyl. In some embodiments, R₁₆ is —PO₃WY. In some embodiments, R₁₆ is —CH₂PO₄WY. In some embodiments, R₁₆ is —CH₂PO₄Z. In some embodiments, R₁₆ is —PO₃Z.

In some embodiments, R₁₇ is hydrogen. In some embodiments, R₁₇ is hydroxy. In some embodiments, R₁₇ is carboxaldehyde. In some embodiments, R₁₇ is unsubstituted amine. In some embodiments, R₁₇ is substituted amine. In some embodiments, R₁₇ is unsubstituted C₁-C₁₀ alkyl. In some embodiments, R₁₇ is unsubstituted C₂-C₁₀ alkynyl. In some embodiments, R₁₇ is substituted C₂-C₁₀ alkynyl. In some embodiments, R₁₇ is unsubstituted C₂-C₁₀ alkenyl. In some embodiments, R₁₇ is substituted C₂-C₁₀ alkenyl. In some embodiments, R₁₇ is carboxyl. In some embodiments, R₁₇ is unsubstituted carbohydrate. In some embodiments, R₁₇ is substituted carbohydrate. In some embodiments, R₁₇ is unsubstituted ester. In some embodiments, R₁₇ is substituted ester. In some embodiments, R₁₇ is unsubstituted acyloxy. In some embodiments, R₁₇ is substituted acyloxy. In some embodiments, R₁₇ is nitro. In some embodiments, R₁₇ is halogen. In some embodiments, R₁₇ is unsubstituted C₁-C₁₀ aliphatic acyl. In some embodiments, R₁₇ is substituted C₁-C₁₀ aliphatic acyl. In some embodiments, R₁₇ is unsubstituted C₆-C₁₀ aromatic acyl. In some embodiments, R₁₇ is substituted C₆-C₁₀ aromatic acyl. In some embodiments, R₁₇ is unsubstituted C₆-C₁₀ aralkyl acyl. In some embodiments, R₁₇ is substituted C₆-C₁₀ aralkyl acyl. In some embodiments, R₁₇ is unsubstituted C₆-C₁₀ alkylaryl acyl. n some embodiments, R₁₇ is substituted C₆-C₁₀ alkylaryl acyl. In some embodiments, R₁₇ is unsubstituted alkoxy. In some embodiments, R₁₇ is substituted alkoxy. In some embodiments, R₁₇ is unsubstituted aryl. In some embodiments, R₁₇ is substituted aryl. In some embodiments, R₁₇ is unsubstituted C₃-C₁₀heterocyclyl. In some embodiments, R₁₇ is substituted C₃-C₁₀heterocyclyl. In some embodiments, R₁₇ is unsubstituted heteroaryl. In some embodiments, R₁₇ is substituted heteroaryl. In some embodiments, R₁₇ is unsubstituted C₃-C₁₀cycloalkyl. In some embodiments, R₁₇ is substituted C₃-C₁₀cycloalkyl. In some embodiments, R₁₇ is —OPO₃WY. In some embodiments, R₁₇ is —OCH₂PO₄WY. In some embodiments, R₁₇ is —OCH₂PO₄Z. In some embodiments, R₁₇ is —OPO₃Z.

In some embodiments, R₁₈ is hydrogen. In some embodiments, R₁₈ is hydroxy. In some embodiments, R₁₈ is carboxaldehyde. In some embodiments, R₁₈ is unsubstituted amine. In some embodiments, R₁₈ is substituted amine. In some embodiments, R₁₈ is unsubstituted C₁-C₁₀ alkyl. In some embodiments, R₁₈ is unsubstituted C₂-C₁₀ alkynyl. In some embodiments, R₁₈ is substituted C₂-C₁₀ alkynyl. In some embodiments, R₁₈ is unsubstituted C₂-C₁₀ alkenyl. In some embodiments, R₁₈ is substituted C₂-C₁₀ alkenyl. In some embodiments, R₁₈ is carboxyl. In some embodiments, R₁₈ is unsubstituted carbohydrate. In some embodiments, R₁₈ is substituted carbohydrate. In some embodiments, R₁₈ is substituted carbohydrate. In some embodiments, R₁₈ is unsubstituted ester. In some embodiments, R₁₈ is substituted ester. In some embodiments, R₁₈ is unsubstituted acyloxy. In some embodiments, R₁₈ is substituted acyloxy. In some embodiments, R₁₈ is nitro. In some embodiments, R₁₈ is halogen. In some embodiments, R₁₈ is unsubstituted C₁-C₁₀ aliphatic acyl. In some embodiments, R₁₈ is substituted C₁-C₁₀ aliphatic acyl. In some embodiments, R₁₈ is unsubstituted C₆-C₁₀ aromatic acyl. In some embodiments, R₁₈ is substituted C₆-C₁₀ aromatic acyl. In some embodiments, R₁₈ is unsubstituted C₆-C₁₀ aralkyl acyl. In some embodiments, R₁₈ is substituted C₆-C₁₀ aralkyl acyl. In some embodiments, R₁₈ is unsubstituted C₆-C₁₀ alkylaryl acyl. In some embodiments, R₁₈ is substituted C₆-C₁₀ alkylaryl acyl. In some embodiments, R₁₈ is unsubstituted alkoxy. In some embodiments, R₁₈ is substituted alkoxy. In some embodiments, R₁₈ is unsubstituted aryl. In some embodiments, R₁₈ is substituted aryl. In some embodiments, R₁₈ is unsubstituted C₃-C₁₀heterocyclyl. In some embodiments, R₁₈ is substituted C₃-C₁₀heterocyclyl. In some embodiments, R₁₈ is unsubstituted heteroaryl. In some embodiments, R₁₈ is substituted heteroaryl. In some embodiments, R₁₈ is unsubstituted C₃-C₁₀cycloalkyl. In some embodiments, R₁₈ is substituted C₃-C₁₀cycloalkyl. In some embodiments, R₁₈ is —OPO₃WY. In some embodiments, R₁₈ is —OCH₂PO₄WY. In some embodiments, R₁₈ is —OCH₂PO₄Z. In some embodiments, R₁₈ is —OPO₃Z.

In some embodiments, R₁₉ is hydrogen. In some embodiments, R₁₉ is unsubstituted C₁-C₁₀ alkyl. In some embodiments, R₁₉ is substituted C₁-C₁₀ alkyl. In some embodiments, R₁₉ is unsubstituted C₂-C₁₀ alkynyl. In some embodiments, R₁₉ is substituted C₂-C₁₀ alkynyl. In some embodiments, R₁₉ is unsubstituted C₂-C₁₀ alkenyl. In some embodiments, R₁₉ is substituted C₂-C₁₀ alkenyl. In some embodiments, R₁₉ is unsubstituted carbohydrate. In some embodiments, R₁₉ is substituted carbohydrate. In some embodiments, R₁₉ is unsubstituted C₁-C₁₀ aliphatic acyl. In some embodiments, R₁₉ is substituted C₁-C₁₀ aliphatic acyl. In some embodiments, R₁₉ is unsubstituted C₆-C₁₀ aromatic acyl. In some embodiments, R₁₉ is substituted C₆-C₁₀ aromatic acyl. In some embodiments, R₁₉ is unsubstituted C₆-C₁₀ aralkyl acyl. In some embodiments, R₁₉ is substituted C₆-C₁₀ aralkyl acyl. In some embodiments, R₁₉ is unsubstituted C₆-C₁₀ alkylaryl acyl. In some embodiments, R₁₉ is substituted C₆-C₁₀ alkylaryl acyl. In some embodiments, R₁₉ is unsubstituted aryl. In some embodiments, R₁₉ is substituted aryl. In some embodiments, R₁₉ is unsubstituted C₃-C₁₀heterocyclyl. In some embodiments, R₁₉ is substituted C₃-C₁₀ heterocyclyl. In some embodiments, R₁₉ is unsubstituted heteroaryl. In some embodiments, R₁₉ is substituted heteroaryl. In some embodiments, R₁₉ is unsubstituted C₃-C₁₀cycloalkyl. In some embodiments, R₁₉ is substituted C₃-C₁₀cycloalkyl. In some embodiments, R₁₉ is —PO₃WY. In some embodiments, R₁₉ is —CH₂PO₄WY. In some embodiments, R₁₉ is —CH₂PO₄Z. In some embodiments, R₁₉ is —PO₃Z.

In some embodiments, R₂₁ is hydrogen. In some embodiments, R₂₁ is hydroxy. In some embodiments, R₂₁ is carboxaldehyde. In some embodiments, R₂₁ is unsubstituted amine. In some embodiments, R₂₁ is substituted amine. In some embodiments, R₂₁ is unsubstituted C₁-C₁₀ alkyl. In some embodiments, R₂₁ is unsubstituted C₂-C₁₀ alkynyl. In some embodiments, R₂₁ is substituted C₂-C₁₀ alkynyl. In some embodiments, R₂₁ is unsubstituted C₂-C₁₀ alkenyl. In some embodiments, R₂₁ is substituted C₂-C₁₀ alkenyl. In some embodiments, R₂₁ is carboxyl. In some embodiments, R₂₁ is unsubstituted carbohydrate. In some embodiments, R₂₁ is substituted carbohydrate. In some embodiments, R₂₁ is unsubstituted ester. In some embodiments, R₂₁ is substituted ester. In some embodiments, R₂₁ is unsubstituted acyloxy. In some embodiments, R₂₁ is substituted acyloxy. In some embodiments, R₂₁ is nitro. In some embodiments, R₂₁ is halogen. In some embodiments, R₂₁ is unsubstituted C₁-C₁₀ aliphatic acyl. In some embodiments, R₂₁ is substituted C₁-C₁₀ aliphatic acyl. In some embodiments, R₂₁ is unsubstituted C₆-C₁₀ aromatic acyl. In some embodiments, R₂₁ is substituted C₆-C₁₀ aromatic acyl. In some embodiments, R₂₁ is unsubstituted C₆-C₁₀ aralkyl acyl. In some embodiments, R₂₁ is substituted C₆-C₁₀ aralkyl acyl. In some embodiments, R₂₁ is unsubstituted C₆-C₁₀ alkylaryl acyl. In some embodiments, R₂₁ is substituted C₆-C₁₀ alkylaryl acyl. In some embodiments, R₂₁ is unsubstituted alkoxy. In some embodiments, R₂₁ is substituted alkoxy. In some embodiments, R₂₁ is unsubstituted aryl. In some embodiments, R₂₁ is substituted aryl. In some embodiments, R₂₁ is unsubstituted C₃-C₁₀heterocyclyl. In some embodiments, R₂₁ is substituted C₃-C₁₀heterocyclyl. In some embodiments, R₂₁ is unsubstituted heteroaryl. In some embodiments, R₂₁ is substituted heteroaryl. In some embodiments, R₂₁ is unsubstituted C₃-C₁₀cycloalkyl. In some embodiments, R₂₁ is substituted C₃-C₁₀cycloalkyl. In some embodiments, R₂₁ is —OPO₃WY. In some embodiments, R₂₁ is —OCH₂PO₄WY. In some embodiments, R₂₁ is —OCH₂PO₄Z. In some embodiments, R₂₁ is —OPO₃Z.

In some embodiments, s is an integer of 0. In some embodiments, s is an integer of 1. In some embodiments, s is an integer of 2. In some embodiments, s is an integer of 3.

In some embodiments, n is an integer of 0. In some embodiments, n is an integer of 1. In some embodiments, n is an integer of 2. In some embodiments, n is an integer of 3. In some embodiments, n is an integer of 4.

In various embodiments, W is hydrogen. In various embodiments, W is unsubstituted methyl. In various embodiments, W is substituted methyl. In various embodiments, W is unsubstituted ethyl. In various embodiments, W is substituted ethyl. In various embodiments, W is unsubstituted alkyl. In various embodiments, W is substituted alkyl. In various embodiments, W is unsubstituted carbohydrate. In various embodiments, W is substituted carbohydrate. In various embodiments, W is potassium. In various embodiments, W is sodium. In various embodiments, W is lithium.

In various embodiments, Y is hydrogen. In various embodiments, Y is unsubstituted methyl. In various embodiments, Y is substituted methyl. In various embodiments, Y is unsubstituted ethyl. In various embodiments, Y is substituted ethyl. In various embodiments, Y is unsubstituted alkyl. In various embodiments, Y is substituted alkyl. In various embodiments, Y is unsubstituted carbohydrate. In various embodiments, Y is substituted carbohydrate. In various embodiments, Y is potassium. In various embodiments, Y is sodium. In various embodiments, Y is lithium.

In various embodiments, Z is calcium. In various embodiments, Z is magnesium. In various embodiments, Z is iron.

In some embodiments, R₅ is hydrogen. In some embodiments, R₅ is hydroxyl. In some embodiments, R₅ is carboxaldehyde. In some embodiments, R₅ is unsubstituted amine. In some embodiments, R₅ is substituted amine. In some embodiments, R₅ is unsubstituted C₁-C₁₀ alkyl. In some embodiments, R₅ is substituted C₁-C₁₀ alkyl. In some embodiments, R₅ is unsubstituted C₂-C₁₀ alkynyl. In some embodiments, R₅ is substituted C₂-C₁₀ alkynyl. In some embodiments, R₅ is unsubstituted C₂-C₁₀ alkenyl. In some embodiments, R₅ is substituted C₂-C₁₀ alkenyl. In some embodiments, R₅ is carboxyl. In some embodiments, R₅ is unsubstituted carbohydrate. In some embodiments, R₅ is substituted carbohydrate. In some embodiments, R₅ is unsubstituted ester. In some embodiments, R₅ is substituted ester. In some embodiments, R₅ is unsubstituted acyloxy. In some embodiments, R₅ is substituted acyloxy. In some embodiments, R₅ is nitro. In some embodiments, R₅ is halogen. In some embodiments, R₅ is unsubstituted C₁-C₁₀ aliphatic acyl. In some embodiments, R₅ is substituted C₁-C₁₀ aliphatic acyl. In some embodiments, R₅ is unsubstituted C₆-C₁₀ aromatic acyl. In some embodiments, R₅ is substituted C₆-C₁₀ aromatic acyl. In some embodiments, R₅ is unsubstituted C₆-C₁₀ aralkyl acyl. In some embodiments, R₅ is substituted C₆-C₁₀ aralkyl acyl. In some embodiments, R₅ is unsubstituted C₆-C₁₀ alkylaryl acyl. In some embodiments, R₅ is substituted C₆-C₁₀ alkylaryl acyl. In some embodiments, R₅ is unsubstituted alkoxy. In some embodiments, R₅ is substituted alkoxy. In some embodiments, R₅ is unsubstituted aryl. In some embodiments, R₅ is substituted aryl. In some embodiments, R₅ is unsubstituted C₃-C₁₀heterocyclyl. In some embodiments, R₅ is substituted C₃-C₁₀ heterocyclyl. In some embodiments, R₅ is unsubstituted heteroaryl, In some embodiments, R₅ is unsubstituted C₃-C₁₀cycloalkyl. In some embodiments, R₅ is substituted C₃-C₁₀cycloalkyl. In some embodiments, R₅ is —OPO₃WY. In some embodiments, R₅ is —OCH₂PO₄—Y. In some embodiments, R₅ is —OCH₂PO₄Z. In some embodiments, R₅ is —OPO₃Z.

In various embodiments of the invention, the pyrone analog is of Formulae III, IV, V, or VI as illustrated in Scheme I.

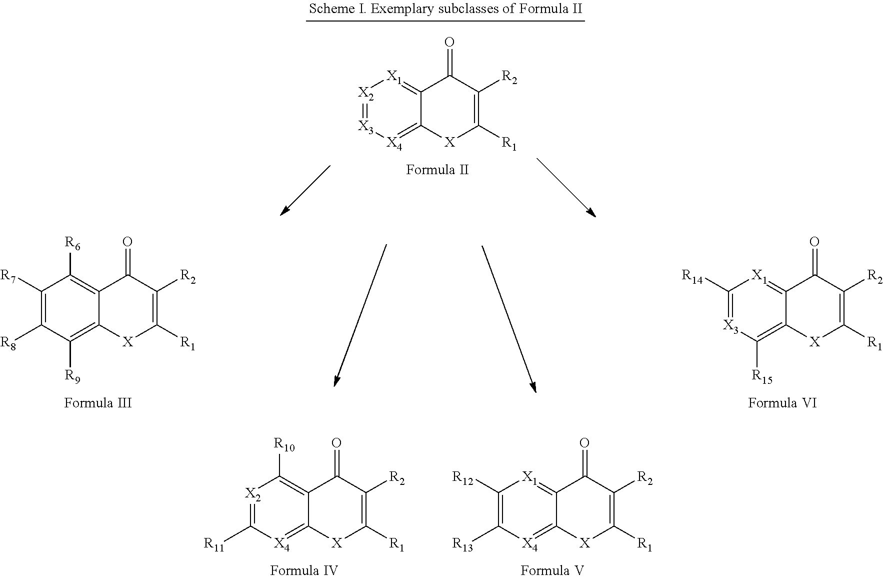

In some embodiments of the invention where X₁, X₂, X₃, and X₄ of Formula II are CR₅, the compound is of Formula III or a pharmaceutically/veterinarily acceptable salt thereof:

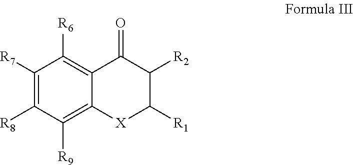

wherein X is O, S, or NR′, wherein R′ is hydrogen, C₁-C₁₀ alkyl, C₂-C₁₀ alkynyl, C₂-C₁₀ alkenyl, C₁-C₁₀ aliphatic acyl, C₆-C₁₀ aromatic acyl, C₆-C₁₀ aralkyl acyl, C₆-C₁₀ alkylaryl acyl, aryl, C₃-C₁₀ heterocyclyl, heteroaryl, or C₃-C₁₀cycloalkyl;

R₁, and R₂ are independently hydrogen, hydroxyl, C₁-C₁₀ alkyl, C₂-C₁₀ alkynyl, C₂-C₁₀ alkenyl, carboxyl, carbohydrate, ester, acyloxy, nitro, halogen, C₁-C₁₀ aliphatic acyl, C₆-C₁₀ aromatic acyl, C₆-C₁₀ aralkyl acyl, C₆-C₁₀ alkylaryl acyl, alkoxy, amine, aryl, C₄-C₁₀heterocyclyl, heteroaryl, C₃-C₁₀cycloalkyl, —OPO₃WY, —OCH₂PO₄WY, —OCH₂PO₄Z or —OPO₃Z;

R₆, R₇, R₈, and R₉ are independently hydrogen, hydroxyl, carboxaldehyde, amino, C₁-C₁₀ alkyl, C₂-C₁₀ alkynyl, C₂-C₁₀ alkenyl, carboxyl, carbohydrate, ester, acyloxy, nitro, halogen, C₁-C₁₀ aliphatic acyl, C₆-C₁₀ aromatic acyl, C₆-C₁₀ aralkyl acyl, C₆-C₁₀alkylaryl acyl, alkoxy, amine, aryl, C₃-C₁₀heterocyclyl, heteroaryl, C₃-C₁₀cycloalkyl, —OPO₃WY, —OCH₂PO₄WY, —OCH₂PO₄Z or —OPO₃Z; and

W and Y are independently hydrogen, methyl, ethyl, alkyl, carbohydrate, or a cation, and Z is a multivalent cation;

wherein the compound of Formula III is not the compound of Formula A:

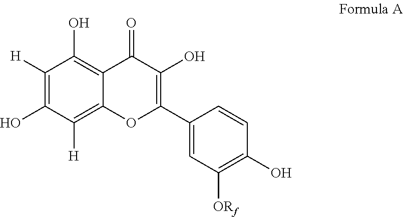

wherein R_{f} is hydrogen; and

wherein the compound of Formula III is not one of the following compounds:

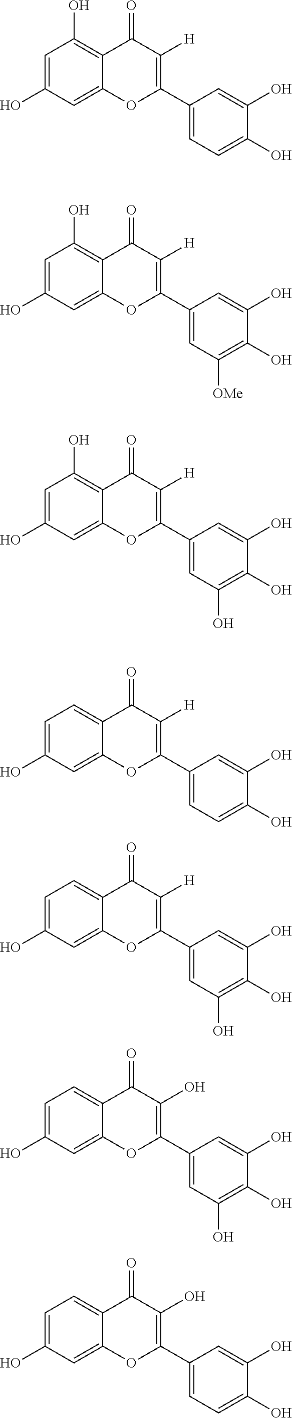

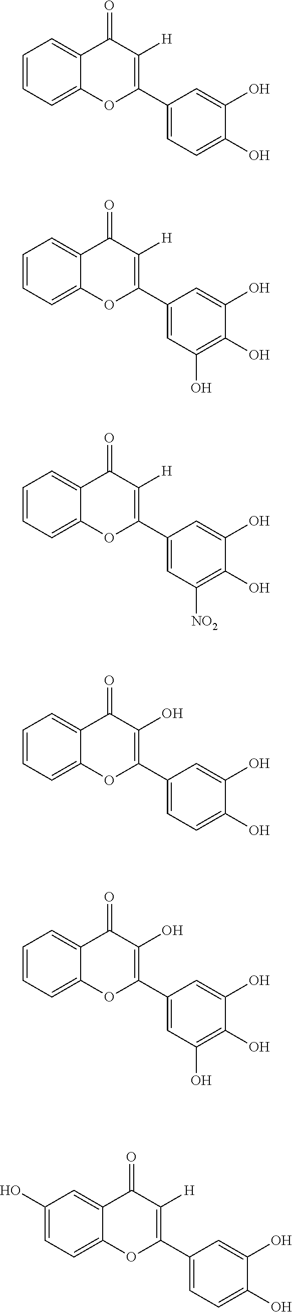

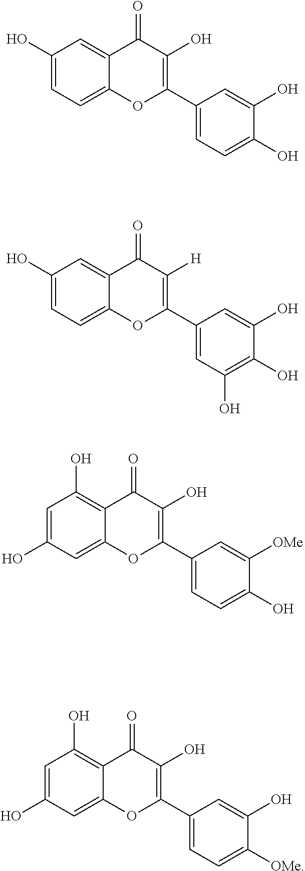

In some embodiments, the compound of Formula III is not the compound of Formula A wherein R_{f} is PO₃K₂.

In some embodiments, X is O.

In other embodiments, X is S.

In yet other embodiments, X is NR′.

In some embodiments, R′ is hydrogen. In some embodiments, R′ is unsubstituted C₁-C₁₀ alkyl. In some embodiments, R′ is substituted C₁-C₁₀ alkyl. In some embodiments, R′ is unsubstituted C₂-C₁₀ alkynyl. In some embodiments, R′ is substituted C₂-C₁₀ alkynyl. In some embodiments, R′ is unsubstituted C₂-C₁₀ alkenyl. In some embodiments, R′ is substituted C₂-C₁₀ alkenyl. In some embodiments, R′ is unsubstituted C₂-C₁₀ alkenyl. In some embodiments, R′ is substituted C₂-C₁₀ alkenyl. In some embodiments, R′ is unsubstituted C₁-C₁₀ aliphatic acyl. In some embodiments, R′ is substituted C₁-C₁₀ aliphatic acyl. In some embodiments, R′ is unsubstituted C₆-C₁₀ aromatic acyl. In some embodiments, R′ is substituted C₆-C₁₀ aromatic acyl. In some embodiments, R′ is unsubstituted C₆-C₁₀ aralkyl acyl. In some embodiments, R′ is substituted C₆-C₁₀ aralkyl acyl. In some embodiments, R′ is unsubstituted C₆-C₁₀ alkylaryl acyl. In some embodiments, R′ is substituted C₆-C₁₀ alkylaryl acyl. In some embodiments, R′ is unsubstituted aryl. In some embodiments, R′ is substituted aryl. In some embodiments, R′ is unsubstituted C₃-C₁₀ heterocyclyl. In some embodiments, R′ is substituted C₃-C₁₀ heterocyclyl. In some embodiments, R′ is unsubstituted heteroaryl. In some embodiments, R′ is substituted heteroaryl. In some embodiments, R′ is unsubstituted C₃-C₁₀cycloalkyl. In some embodiments, R′ is substituted C₃-C₁₀cycloalkyl.

In some embodiments, R₁ is hydrogen. In some embodiments, R₁ is hydroxyl. In some embodiments, R₁ is optionally substituted C₁-C₁₀ alkyl. In some embodiments, R₁ is unsubstituted C₁-C₁₀ alkyl. In some embodiments, R₁ is substituted C₁-C₁₀ alkyl. In some embodiments, R₁ is unsubstituted C₁-C₁₀ alkyl. In some other embodiments, R₁ is substituted C₁-C₁₀ alkyl. In some embodiments, R₁ is unsubstituted C₂-C₁₀ alkynyl. In some embodiments, R₁ is substituted C₂-C₁₀ alkynyl. In some embodiments, R₁ is unsubstituted C₂-C₁₀ alkenyl. In some embodiments, R₁ is substituted C₂-C₁₀ alkenyl. In some embodiments, R₁ is carboxyl. In some embodiments, R₁ is unsubstituted carbohydrate. In some embodiments, R₁ is substituted carbohydrate. In some embodiments, R₁ is unsubstituted ester. In some embodiments, R₁ is substituted ester. In some embodiments, R₁ is unsubstituted acyloxy. In some embodiments, R₁ is substituted acyloxy. In some embodiments, R₁ is nitro. In some embodiments, R₁ is halogen. In some embodiments, R₁ is unsubstituted C₁-C₁₀ aliphatic acyl. In some embodiments, R₁ is substituted C₁-C₁₀ aliphatic acyl. In some embodiments, R₁ is unsubstituted C₆-C₁₀ aromatic acyl. In some embodiments, R₁ is substituted C₆-C₁₀ aromatic acyl. In some embodiments, R₁ is unsubstituted C₆-C₁₀ aralkyl acyl. In some embodiments, R₁ is substituted C₆-C₁₀ aralkyl acyl. In some embodiments, R₁ is unsubstituted C₆-C₁₀ alkylaryl acyl. In some embodiments, R₁ is substituted C₆-C₁₀ alkylaryl acyl. In some embodiments, R₁ is unsubstituted alkoxy. In some embodiments, R₁ is substituted alkoxy. In some embodiments, R₁ is unsubstituted amine. In some embodiments, R₁ is substituted amine. In some embodiments, R₁ is unsubstituted aryl. In some embodiments, R₁ is substituted aryl. In some embodiments, R₁ is unsubstituted C₄-C₁₀heterocyclyl. In some embodiments, R₁ is substituted C₄-C₁₀heterocyclyl. In some embodiments, R₁ is unsubstituted heteroaryl. In some embodiments, R₁ is substituted heteroaryl. In some embodiments, R₁ is unsubstituted C₃-C₁₀cycloalkyl. In some embodiments, R₁ is substituted C₃-C₁₀cycloalkyl. In some embodiments, R₁ is —OPO₃WY. In some embodiments, R₁ is —OCH₂PO₄WY. In some embodiments, R₁ is —OCH₂PO₄Z. In some embodiments, R₁ is —OPO₃Z.

In some embodiments, R₂ is hydrogen. In some embodiments, R₂ is hydroxyl. In some embodiments, R₂ is optionally substituted C₁-C₁₀ alkyl. In some embodiments, R₂ is unsubstituted C₁-C₁₀ alkyl. In some embodiments, R₂ is substituted C₁-C₁₀ alkyl. In some embodiments, R₂ is unsubstituted C₁-C₁₀ alkyl. In some other embodiments, R₂ is substituted C₁-C₁₀ alkyl. In some embodiments, R₂ is unsubstituted C₂-C₁₀ alkynyl. In some embodiments, R₂ is substituted C₂-C₁₀ alkynyl. In some embodiments, R₂ is unsubstituted C₂-C₁₀ alkenyl. In some embodiments, R₂ is substituted C₂-C₁₀ alkenyl. In some embodiments, R₂ is carboxyl. In some embodiments, R₂ is unsubstituted carbohydrate. In some embodiments, R₂ is substituted carbohydrate. In some embodiments, R₂ is unsubstituted ester. In some embodiments, R₂ is substituted ester. In some embodiments, R₂ is unsubstituted acyloxy. In some embodiments, R₂ is substituted acyloxy. In some embodiments, R₂ is nitro. In some embodiments, R₂ is halogen. In some embodiments, R₂ is unsubstituted C₁-C₁₀ aliphatic acyl. In some embodiments, R₂ is substituted C₁-C₁₀ aliphatic acyl. In some embodiments, R₂ is unsubstituted C₆-C₁₀ aromatic acyl. In some embodiments, R₂ is substituted C₆-C₁₀ aromatic acyl. In some embodiments, R₂ is unsubstituted C₆-C₁₀ aralkyl acyl. In some embodiments, R₂ is substituted C₆-C₁₀ aralkyl acyl. In some embodiments, R₂ is unsubstituted C₆-C₁₀ alkylaryl acyl. In some embodiments, R₂ is substituted C₆-C₁₀ alkylaryl acyl. In some embodiments, R₂ is unsubstituted alkoxy. In some embodiments, R₂ is substituted alkoxy. In some embodiments, R₂ is unsubstituted amine. In some embodiments, R₂ is substituted amine. In some embodiments, R₂ is unsubstituted aryl. In some embodiments, R₂ is substituted aryl. In some embodiments, R₂ is unsubstituted C₄-C₁₀heterocyclyl. In some embodiments, R₂ is substituted C₄-C₁₀heterocyclyl. In some embodiments, R₂ is unsubstituted heteroaryl. In some embodiments, R₂ is substituted heteroaryl. In some embodiments, R₂ is unsubstituted C₃-C₁₀cycloalkyl. In some embodiments, R₂ is substituted C₃-C₁₀cycloalkyl. In some embodiments, R₂ is —OPO₃WY. In some embodiments, R₂ is —OCH₂PO₄WY. In some embodiments, R₂ is —OCH₂PO₄Z. In some embodiments, R₂ is —OPO₃Z.

In some embodiments, when R₁ is aryl, it is monocyclic. In some embodiments, when R₁ is aryl, it is bicyclic. In some embodiments, when R₁ is heteroaryl, it is monocyclic. In some embodiments, when R₁ is heteroaryl, it is bicyclic.

In various embodiments, R₁ is one of the following formulae:

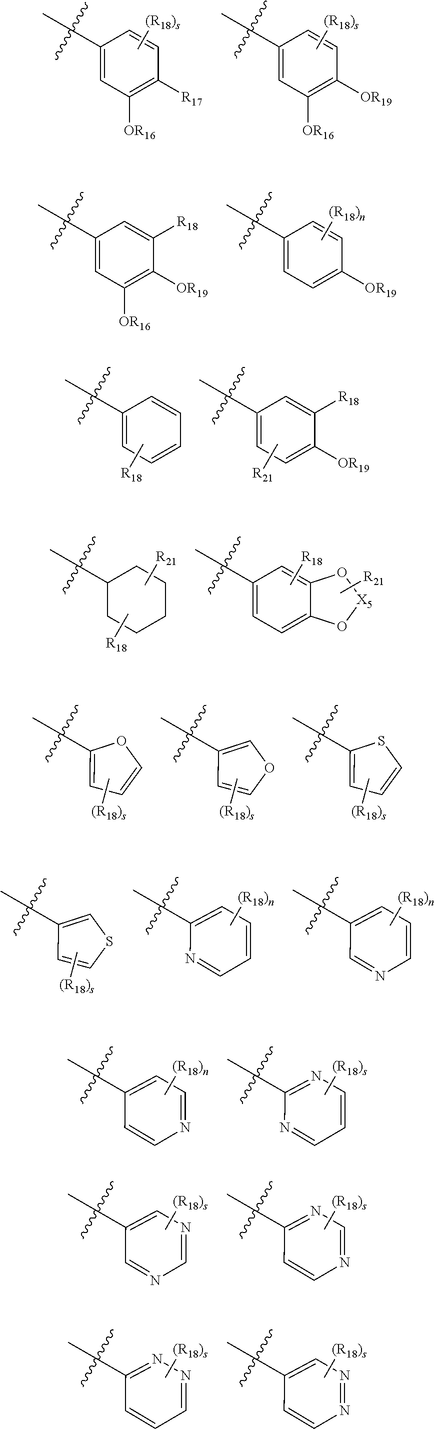

wherein R₁₆ is hydrogen, C₁-C₁₀ alkyl, C₂-C₁₀ alkynyl, C₂-C₁₀ alkenyl, carbohydrate, C₁-C₁₀ aliphatic acyl, C₆-C₁₀ aromatic acyl, C₆-C₁₀ aralkyl acyl, C₆-C₁₀ alkylaryl acyl, aryl, C₃-C₁₀ heterocyclyl, heteroaryl, C₃-C₁₀cycloalkyl, —PO₃WY, —CH₂PO₄WY, —CH₂PO₄Z or —PO₃Z;

R₁₇ is hydrogen, hydroxy, carboxaldehyde, amine, C₁-C₁₀ alkyl, C₂-C₁₀ alkynyl, C₂-C₁₀ alkenyl, carboxyl, carbohydrate, ester, acyloxy, nitro, halogen, C₁-C₁₀ aliphatic acyl, C₆-C₁₀ aromatic acyl, C₆-C₁₀ aralkyl acyl, C₆-C₁₀ alkylaryl acyl, alkoxy, aryl, C₃-C₁₀ heterocyclyl, heteroaryl, or C₃-C₁₀cycloalkyl, —OPO₃WY, —OCH₂PO₄WY, —OCH₂PO₄Z or —OPO₃Z;

each instance of R₁₈ and R₂₁ is independently hydrogen, hydroxyl, carboxaldehyde, amine, C₁-C₁₀ alkyl, C₂-C₁₀ alkynyl, C₂-C₁₀ alkenyl, carboxyl, carbohydrate, ester, acyloxy, nitro, halogen, C₁-C₁₀ aliphatic acyl, C₆-C₁₀ aromatic acyl, C₆-C₁₀ aralkyl acyl, C₆-C₁₀ alkylaryl acyl, alkoxy, alkyl, phosphate, aryl, heteroaryl, C₃-C₁₀ heterocyclic, C₃-C₁₀cycloalkyl, —OPO₃WY, —OCH₂PO₄WY, —OCH₂PO₄Z or —OPO₃Z;

R₁₉ is hydrogen, C₁-C₁₀ alkyl, C₂-C₁₀ alkynyl, C₂-C₁₀ alkenyl, carbohydrate, C₁-C₁₀ aliphatic acyl, C₆-C₁₀ aromatic acyl, C₆-C₁₀ aralkyl acyl, C₆-C₁₀ alkylaryl acyl, aryl, C₃-C₁₀heterocyclyl, heteroaryl, optionally substituted C₃-C₁₀cycloalkyl, —PO₃WY, —CH₂PO₄WY, —CH₂PO₄Z or —PO₃Z;

s is an integer of 0, 1, 2, or 3; and

n is an integer of 0, 1, 2, 3, or 4.

In some embodiments of the invention, at least one of R₁₆ and R₁₉ is —PO₃WY, —CH₂PO₄WY, —CH₂PO₄Z or —PO₃Z, or at least one of R₁₇ or R₁₈ is —OPO₃WY, —OCH₂PO₄WY, —OCH₂PO₄Z or —OPO₃Z. In some embodiments, at least one of R₁₆ and R₁₉ is —PO₃WY, —CH₂PO₄WY, —CH₂PO₄Z or —PO₃Z and at least one of R₁₇ or R₁₈ is —OPO₃WY, —OCH₂PO₄WY, —OCH₂PO₄Z or —OPO₃Z.

In some embodiments, R₆ is hydrogen. In some embodiments, R₆ is hydroxyl. In some embodiments, R₆ is carboxaldehyde. In some embodiments, R₆ is unsubstituted amine. In some embodiments, R₆ is substituted amine. In some embodiments, R₆ is unsubstituted C₁-C₁₀ alkyl. In some embodiments, R₆ is substituted C₁-C₁₀ alkyl. In some embodiments, R₆ is unsubstituted C₂-C₁₀ alkynyl. In some embodiments, R₆ is substituted C₂-C₁₀ alkynyl. In some embodiments, R₆ is unsubstituted C₂-C₁₀ alkenyl. In some embodiments, R₆ is substituted C₂-C₁₀ alkenyl. In some embodiments, R₆ is carboxyl. In some embodiments, R₆ is unsubstituted carbohydrate. In some embodiments, R₆ is substituted carbohydrate. In some embodiments, R₆ is unsubstituted ester. In some embodiments, R₆ is substituted ester. In some embodiments, R₆ is unsubstituted acyloxy. In some embodiments, R₆ is substituted acyloxy. In some embodiments, R₆ is nitro. In some embodiments, R₆ is halogen. In some embodiments, R₆ is unsubstituted C₁-C₁₀ aliphatic acyl. In some embodiments, R₆ is substituted C₁-C₁₀ aliphatic acyl. In some embodiments, R₆ is unsubstituted C₆-C₁₀ aromatic acyl. In some embodiments, R₆ is substituted C₆-C₁₀ aromatic acyl. In some embodiments, R₆ is unsubstituted C₆-C₁₀ aralkyl acyl. In some embodiments, R₆ is substituted C₆-C₁₀ aralkyl acyl. In some embodiments, R₆ is unsubstituted C₆-C₁₀ alkylaryl acyl. In some embodiments, R₆ is substituted C₆-C₁₀ alkylaryl acyl. In some embodiments, R₆ is unsubstituted alkoxy. In some embodiments, R₆ is substituted alkoxy. In some embodiments, R₆ is unsubstituted aryl. In some embodiments, R₆ is substituted aryl. In some embodiments, R₆ is unsubstituted C₃-C₁₀heterocyclyl. In some embodiments, R₆ is substituted C₃-C₁₀ heterocyclyl. In some embodiments, R₆ is unsubstituted heteroaryl. In some embodiments, R₆ is unsubstituted C₃-C₁₀cycloalkyl. In some embodiments, R₆ is substituted C₃-C₁₀cycloalkyl. In some embodiments, R₆ is —OPO₃WY. In some embodiments, R₆ is —OCH₂PO₄WY. In some embodiments, R₆ is —OCH₂PO₄Z. In some embodiments, R₆ is —OPO₃Z.

In some embodiments, R₇ is hydrogen. In some embodiments, R₇ is hydroxyl. In some embodiments, R₇ is carboxaldehyde. In some embodiments, R₇ is unsubstituted amine. In some embodiments, R₇ is substituted amine. In some embodiments, R₇ is unsubstituted C₁-C₁₀ alkyl. In some embodiments, R₇ is substituted C₁-C₁₀ alkyl. In some embodiments, R₇ is unsubstituted C₂-C₁₀ alkynyl. In some embodiments, R₇ is substituted C₂-C₁₀ alkynyl. In some embodiments, R₇ is unsubstituted C₂-C₁₀ alkenyl. In some embodiments, R₇ is substituted C₂-C₁₀ alkenyl. In some embodiments, R₇ is carboxyl. In some embodiments, R₇ is unsubstituted carbohydrate. In some embodiments, R₇ is substituted carbohydrate. In some embodiments, R₇ is unsubstituted ester. In some embodiments, R₇ is substituted ester. In some embodiments, R₇ is unsubstituted acyloxy. In some embodiments, R₇ is substituted acyloxy. In some embodiments, R₇ is nitro. In some embodiments, R₇ is halogen. In some embodiments, R₇ is unsubstituted C₁-C₁₀ aliphatic acyl. In some embodiments, R₇ is substituted C₁-C₁₀ aliphatic acyl. In some embodiments, R₇ is unsubstituted C₆-C₁₀ aromatic acyl. In some embodiments, R₇ is substituted C₆-C₁₀ aromatic acyl. In some embodiments, R₇ is unsubstituted C₆-C₁₀ aralkyl acyl. In some embodiments, R₇ is substituted C₆-C₁₀ aralkyl acyl. In some embodiments, R₇ is unsubstituted C₆-C₁₀ alkylaryl acyl. In some embodiments, R₇ is substituted C₆-C₁₀ alkylaryl acyl. In some embodiments, R₇ is unsubstituted alkoxy. In some embodiments, R₇ is substituted alkoxy. In some embodiments, R₇ is unsubstituted aryl. In some embodiments, R₇ is substituted aryl. In some embodiments, R₇ is unsubstituted C₃-C₁₀heterocyclyl. In some embodiments, R₇ is substituted C₃-C₁₀ heterocyclyl. In some embodiments, R₇ is unsubstituted heteroaryl, In some embodiments, R₇ is unsubstituted C₃-C₁₀cycloalkyl. In some embodiments, R₇ is substituted C₃-C₁₀cycloalkyl. In some embodiments, R₇ is —OPO₃WY. In some embodiments, R₇ is —OCH₂PO₄WY. In some embodiments, R₇ is —OCH₂PO₄Z. In some embodiments, R₇ is —OPO₃Z.

In some embodiments, R₈ is hydrogen. In some embodiments, R₈ is hydroxyl. In some embodiments, R₈ is carboxaldehyde. In some embodiments, R₈ is unsubstituted amine. In some embodiments, R₈ is substituted amine. In some embodiments, R₈ is unsubstituted C₁-C₁₀ alkyl. In some embodiments, R₈ is substituted C₁-C₁₀ alkyl. In some embodiments, R₈ is unsubstituted C₂-C₁₀ alkynyl. In some embodiments, R₈ is substituted C₂-C₁₀ alkynyl. In some embodiments, R₈ is unsubstituted C₂-C₁₀ alkenyl. In some embodiments, R₈ is substituted C₂-C₁₀ alkenyl. In some embodiments, R₈ is carboxyl. In some embodiments, R₈ is unsubstituted carbohydrate. In some embodiments, R₈ is substituted carbohydrate. In some embodiments, R₈ is unsubstituted ester. In some embodiments, R₈ is substituted ester. In some embodiments, R₈ is unsubstituted acyloxy. In some embodiments, R₈ is substituted acyloxy. In some embodiments, R₈ is nitro. In some embodiments, R₈ is halogen. In some embodiments, R₈ is unsubstituted C₁-C₁₀ aliphatic acyl. In some embodiments, R₈ is substituted C₁-C₁₀ aliphatic acyl. In some embodiments, R₈ is unsubstituted C₆-C₁₀ aromatic acyl. In some embodiments, R₈ is substituted C₆-C₁₀ aromatic acyl. In some embodiments, R₈ is unsubstituted C₆-C₁₀ aralkyl acyl. In some embodiments, R₈ is substituted C₆-C₁₀ aralkyl acyl. In some embodiments, R₈ is unsubstituted C₆-C₁₀ alkylaryl acyl. In some embodiments, R₈ is substituted C₆-C₁₀ alkylaryl acyl. In some embodiments, R₈ is unsubstituted alkoxy. In some embodiments, R₈ is substituted alkoxy. In some embodiments, R₈ is unsubstituted aryl. In some embodiments, R₈ is substituted aryl. In some embodiments, R₈ is unsubstituted C₃-C₁₀heterocyclyl. In some embodiments, R₈ is substituted C₃-C₁₀ heterocyclyl. In some embodiments, R₈ is unsubstituted heteroaryl, In some embodiments, R₈ is unsubstituted C₃-C₁₀cycloalkyl. In some embodiments, R₈ is substituted C₃-C₁₀cycloalkyl. In some embodiments, R₈ is —OPO₃WY. In some embodiments, R₈ is —OCH₂PO₄WY. In some embodiments, R₈ is —OCH₂PO₄Z. In some embodiments, R₈ is —OPO₃Z.

In some embodiments, R₉ is hydrogen. In some embodiments, R₉ is hydroxyl. In some embodiments, R₉ is carboxaldehyde. In some embodiments, R₉ is unsubstituted amine. In some embodiments, R₉ is substituted amine. In some embodiments, R₉ is unsubstituted C₁-C₁₀ alkyl. In some embodiments, R₉ is substituted C₁-C₁₀ alkyl. In some embodiments, R₉ is unsubstituted C₂-C₁₀ alkynyl. In some embodiments, R₉ is substituted C₂-C₁₀ alkynyl. In some embodiments, R₉ is unsubstituted C₂-C₁₀ alkenyl. In some embodiments, R₉ is substituted C₂-C₁₀ alkenyl. In some embodiments, R₉ is carboxyl. In some embodiments, R₉ is unsubstituted carbohydrate. In some embodiments, R₉ is substituted carbohydrate. In some embodiments, R₉ is unsubstituted ester. In some embodiments, R₉ is substituted ester. In some embodiments, R₉ is unsubstituted acyloxy. In some embodiments, R₉ is substituted acyloxy. In some embodiments, R₉ is nitro. In some embodiments, R₉ is halogen. In some embodiments, R₉ is unsubstituted C₁-C₁₀ aliphatic acyl. In some embodiments, R₉ is substituted C₁-C₁₀ aliphatic acyl. In some embodiments, R₉ is unsubstituted C₆-C₁₀ aromatic acyl. In some embodiments, R₉ is substituted C₆-C₁₀ aromatic acyl. In some embodiments, R₉ is unsubstituted C₆-C₁₀ aralkyl acyl. In some embodiments, R₉ is substituted C₆-C₁₀ aralkyl acyl. In some embodiments, R₉ is unsubstituted C₆-C₁₀ alkylaryl acyl. In some embodiments, R₉ is substituted C₆-C₁₀ alkylaryl acyl. In some embodiments, R₉ is unsubstituted alkoxy. In some embodiments, R₉ is substituted alkoxy. In some embodiments, R₉ is unsubstituted aryl. In some embodiments, R₉ is substituted aryl. In some embodiments, R₉ is unsubstituted C₃-C₁₀heterocyclyl. In some embodiments, R₉ is substituted C₃-C₁₀ heterocyclyl. In some embodiments, R₉ is unsubstituted heteroaryl, In some embodiments, R₉ is unsubstituted C₃-C₁₀cycloalkyl. In some embodiments, R₉ is substituted C₃-C₁₀cycloalkyl. In some embodiments, R₉ is —OPO₃WY. In some embodiments, R₉ is —OCH₂PO₄WY. In some embodiments, R₉ is —OCH₂PO₄Z. In some embodiments, R₉ is —OPO₃Z.

In some embodiments, R₁₆ is hydrogen. In some embodiments, R₁₆ is unsubstituted C₁-C₁₀ alkyl. In some embodiments, R₁₆ is substituted C₁-C₁₀ alkyl. In some embodiments, R₁₆ is unsubstituted C₂-C₁₀ alkynyl. In some embodiments, R₁₆ is substituted C₂-C₁₀ alkynyl. In some embodiments, R₁₆ is unsubstituted C₂-C₁₀ alkenyl. In some embodiments, R₁₆ is substituted C₂-C₁₀ alkenyl. In some embodiments, R₁₆ is unsubstituted carbohydrate 1. In some embodiments, R₁₆ is substituted carbohydrate. In some embodiments, R₁₆ is unsubstituted C₁-C₁₀ aliphatic acyl. In some embodiments, R₁₆ is substituted C₁-C₁₀ aliphatic acyl. In some embodiments, R₁₆ is unsubstituted C₆-C₁₀ aromatic acyl. In some embodiments, R₁₆ is substituted C₆-C₁₀ aromatic acyl. In some embodiments, R₁₆ is unsubstituted C₆-C₁₀ aralkyl acyl. In some embodiments, R₁₆ is substituted C₆-C₁₀ aralkyl acyl. In some embodiments, R₁₆ is unsubstituted C₆-C₁₀ alkylaryl acyl. In some embodiments, R₁₆ is substituted C₆-C₁₀ alkylaryl acyl. In some embodiments, R₁₆ is unsubstituted aryl. In some embodiments, R₁₆ is substituted aryl. In some embodiments, R₁₆ is unsubstituted C₃-C₁₀heterocyclyl. In some embodiments, R₁₆ is substituted C₃-C₁₀ heterocyclyl. In some embodiments, R₁₆ is unsubstituted heteroaryl. In some embodiments, R₁₆ is substituted heteroaryl. In some embodiments, R₁₆ is unsubstituted C₃-C₁₀cycloalkyl. In some embodiments, R₁₆ is substituted C₃-C₁₀cycloalkyl. In some embodiments, R₁₆ is —PO₃WY. In some embodiments, R₁₆ is —CH₂PO₄WY. In some embodiments, R₁₆ is —CH₂PO₄Z. In some embodiments, R₁₆ is —PO₃Z.

In some embodiments, R₁₇ is hydrogen. In some embodiments, R₁₇ is hydroxy. In some embodiments, R₁₇ is carboxaldehyde. In some embodiments, R₁₇ is unsubstituted amine. In some embodiments, R₁₇ is substituted amine. In some embodiments, R₁₇ is unsubstituted C₁-C₁₀ alkyl. In some embodiments, R₁₇ is unsubstituted C₂-C₁₀ alkynyl. In some embodiments, R₁₇ is substituted C₂-C₁₀ alkynyl. In some embodiments, R₁₇ is unsubstituted C₂-C₁₀ alkenyl. In some embodiments, R₁₇ is substituted C₂-C₁₀ alkenyl. In some embodiments, R₁₇ is carboxyl. In some embodiments, R₁₇ is unsubstituted carbohydrate. In some embodiments, R₁₇ is substituted carbohydrate. In some embodiments, R₁₇ is unsubstituted ester. In some embodiments, R₁₇ is substituted ester. In some embodiments, R₁₇ is unsubstituted acyloxy. In some embodiments, R₁₇ is substituted acyloxy. In some embodiments, R₁₇ is nitro. In some embodiments, R₁₇ is halogen. In some embodiments, R₁₇ is unsubstituted C₁-C₁₀ aliphatic acyl. In some embodiments, R₁₇ is substituted C₁-C₁₀ aliphatic acyl. In some embodiments, R₁₇ is unsubstituted C₆-C₁₀ aromatic acyl. In some embodiments, R₁₇ is substituted C₆-C₁₀ aromatic acyl. In some embodiments, R₁₇ is unsubstituted C₆-C₁₀ aralkyl acyl. In some embodiments, R₁₇ is substituted C₆-C₁₀ aralkyl acyl. In some embodiments, R₁₇ is unsubstituted C₆-C₁₀ alkylaryl acyl. n some embodiments, R₁₇ is substituted C₆-C₁₀ alkylaryl acyl. In some embodiments, R₁₇ is unsubstituted alkoxy. In some embodiments, R₁₇ is substituted alkoxy. In some embodiments, R₁₇ is unsubstituted aryl. In some embodiments, R₁₇ is substituted aryl. In some embodiments, R₁₇ is unsubstituted C₃-C₁₀heterocyclyl. In some embodiments, R₁₇ is substituted C₃-C₁₀heterocyclyl. In some embodiments, R₁₇ is unsubstituted heteroaryl. In some embodiments, R₁₇ is substituted heteroaryl. In some embodiments, R₁₇ is unsubstituted C₃-C₁₀cycloalkyl. In some embodiments, R₁₇ is substituted C₃-C₁₀cycloalkyl. In some embodiments, R₁₇ is —OPO₃WY. In some embodiments, R₁₇ is —OCH₂PO₄WY. In some embodiments, R₁₇ is —OCH₂PO₄Z. In some embodiments, R₁₇ is —OPO₃Z.

In some embodiments, R₁₈ is hydrogen. In some embodiments, R₁₈ is hydroxy. In some embodiments, R₁₈ is carboxaldehyde. In some embodiments, R₁₈ is unsubstituted amine. In some embodiments, R₁₈ is substituted amine. In some embodiments, R₁₈ is unsubstituted C₁-C₁₀ alkyl. In some embodiments, R₁₈ is unsubstituted C₂-C₁₀ alkynyl. In some embodiments, R₁₈ is substituted C₂-C₁₀ alkynyl. In some embodiments, R₁₈ is unsubstituted C₂-C₁₀ alkenyl. In some embodiments, R₁₈ is substituted C₂-C₁₀ alkenyl. In some embodiments, R₁₈ is carboxyl. In some embodiments, R₁₈ is unsubstituted carbohydrate. In some embodiments, R₁₈ is substituted carbohydrate. In some embodiments, R₁₈ is substituted carbohydrate. In some embodiments, R₁₈ is unsubstituted ester. In some embodiments, R₁₈ is substituted ester. In some embodiments, R₁₈ is unsubstituted acyloxy. In some embodiments, R₁₈ is substituted acyloxy. In some embodiments, R₁₈ is nitro. In some embodiments, R₁₈ is halogen. In some embodiments, R₁₈ is unsubstituted C₁-C₁₀ aliphatic acyl. In some embodiments, R₁₈ is substituted C₁-C₁₀ aliphatic acyl. In some embodiments, R₁₈ is unsubstituted C₆-C₁₀ aromatic acyl. In some embodiments, R₁₈ is substituted C₆-C₁₀ aromatic acyl. In some embodiments, R₁₈ is unsubstituted C₆-C₁₀ aralkyl acyl. In some embodiments, R₁₈ is substituted C₆-C₁₀ aralkyl acyl. In some embodiments, R₁₈ is unsubstituted C₆-C₁₀ alkylaryl acyl. In some embodiments, R₁₈ is substituted C₆-C₁₀ alkylaryl acyl. In some embodiments, R₁₈ is unsubstituted alkoxy. In some embodiments, R₁₈ is substituted alkoxy. In some embodiments, R₁₈ is unsubstituted aryl. In some embodiments, R₁₈ is substituted aryl. In some embodiments, R₁₈ is unsubstituted C₃-C₁₀heterocyclyl. In some embodiments, R₁₈ is substituted C₃-C₁₀heterocyclyl. In some embodiments, R₁₈ is unsubstituted heteroaryl. In some embodiments, R₁₈ is substituted heteroaryl. In some embodiments, R₁₈ is unsubstituted C₃-C₁₀cycloalkyl. In some embodiments, R₁₈ is substituted C₃-C₁₀cycloalkyl. In some embodiments, R₁₈ is —OPO₃WY. In some embodiments, R₁₈ is —OCH₂PO₄WY. In some embodiments, R₁₈ is —OCH₂PO₄Z. In some embodiments, R₁₈ is —OPO₃Z.

In some embodiments, R₁₉ is hydrogen. In some embodiments, R₁₉ is unsubstituted C₁-C₁₀ alkyl. In some embodiments, R₁₉ is substituted C₁-C₁₀ alkyl. In some embodiments, R₁₉ is unsubstituted C₂-C₁₀ alkynyl. In some embodiments, R₁₉ is substituted C₂-C₁₀ alkynyl. In some embodiments, R₁₉ is unsubstituted C₂-C₁₀ alkenyl. In some embodiments, R₁₉ is substituted C₂-C₁₀ alkenyl. In some embodiments, R₁₉ is unsubstituted carbohydrate. In some embodiments, R₁₉ is substituted carbohydrate. In some embodiments, R₁₉ is unsubstituted C₁-C₁₀ aliphatic acyl. In some embodiments, R₁₉ is substituted C₁-C₁₀ aliphatic acyl. In some embodiments, R₁₉ is unsubstituted C₆-C₁₀ aromatic acyl. In some embodiments, R₁₉ is substituted C₆-C₁₀ aromatic acyl. In some embodiments, R₁₉ is unsubstituted C₆-C₁₀ aralkyl acyl. In some embodiments, R₁₉ is substituted C₆-C₁₀ aralkyl acyl. In some embodiments, R₁₉ is unsubstituted C₆-C₁₀ alkylaryl acyl. In some embodiments, R₁₉ is substituted C₆-C₁₀ alkylaryl acyl. In some embodiments, R₁₉ is unsubstituted aryl. In some embodiments, R₁₉ is substituted aryl. In some embodiments, R₁₉ is unsubstituted C₃-C₁₀heterocyclyl. In some embodiments, R₁₉ is substituted C₃-C₁₀ heterocyclyl. In some embodiments, R₁₉ is unsubstituted heteroaryl. In some embodiments, R₁₉ is substituted heteroaryl. In some embodiments, R₁₉ is unsubstituted C₃-C₁₀cycloalkyl. In some embodiments, R₁₉ is substituted C₃-C₁₀cycloalkyl. In some embodiments, R₁₉ is —PO₃WY. In some embodiments, R₁₉ is —CH₂PO₄WY. In some embodiments, R₁₉ is —CH₂PO₄Z. In some embodiments, R₁₉ is —PO₃Z.

In some embodiments, R₂₁ is hydrogen. In some embodiments, R₂₁ is hydroxy. In some embodiments, R₂₁ is carboxaldehyde. In some embodiments, R₂₁ is unsubstituted amine. In some embodiments, R₂₁ is substituted amine. In some embodiments, R₂₁ is unsubstituted C₁-C₁₀ alkyl. In some embodiments, R₂₁ is unsubstituted C₂-C₁₀ alkynyl. In some embodiments, R₂₁ is substituted C₂-C₁₀ alkynyl. In some embodiments, R₂₁ is unsubstituted C₂-C₁₀ alkenyl. In some embodiments, R₂₁ is substituted C₂-C₁₀ alkenyl. In some embodiments, R₂₁ is carboxyl. In some embodiments, R₂₁ is unsubstituted carbohydrate. In some embodiments, R₂₁ is substituted carbohydrate. In some embodiments, R₂₁ is unsubstituted ester. In some embodiments, R₂₁ is substituted ester. In some embodiments, R₂₁ is unsubstituted acyloxy. In some embodiments, R₂₁ is substituted acyloxy. In some embodiments, R₂₁ is nitro. In some embodiments, R₂₁ is halogen. In some embodiments, R₂₁ is unsubstituted C₁-C₁₀ aliphatic acyl. In some embodiments, R₂₁ is substituted C₁-C₁₀ aliphatic acyl. In some embodiments, R₂₁ is unsubstituted C₆-C₁₀ aromatic acyl. In some embodiments, R₂₁ is substituted C₆-C₁₀ aromatic acyl. In some embodiments, R₂₁ is unsubstituted C₆-C₁₀ aralkyl acyl. In some embodiments, R₂₁ is substituted C₆-C₁₀ aralkyl acyl. In some embodiments, R₂₁ is unsubstituted C₆-C₁₀ alkylaryl acyl. In some embodiments, R₂₁ is substituted C₆-C₁₀ alkylaryl acyl. In some embodiments, R₂₁ is unsubstituted alkoxy. In some embodiments, R₂₁ is substituted alkoxy. In some embodiments, R₂₁ is unsubstituted aryl. In some embodiments, R₂₁ is substituted aryl. In some embodiments, R₂₁ is unsubstituted C₃-C₁₀heterocyclyl. In some embodiments, R₂₁ is substituted C₃-C₁₀heterocyclyl. In some embodiments, R₂₁ is unsubstituted heteroaryl. In some embodiments, R₂₁ is substituted heteroaryl. In some embodiments, R₂₁ is unsubstituted C₃-C₁₀cycloalkyl. In some embodiments, R₂₁ is substituted C₃-C₁₀cycloalkyl. In some embodiments, R₂₁ is —OPO₃WY. In some embodiments, R₂₁ is —OCH₂PO₄WY. In some embodiments, R₂₁ is —OCH₂PO₄Z. In some embodiments, R₂₁ is —OPO₃Z.

In some embodiments, s is an integer of 0. In some embodiments, s is an integer of 1. In some embodiments, s is an integer of 2. In some embodiments, s is an integer of 3.

In some embodiments, n is an integer of 0. In some embodiments, n is an integer of 1. In some embodiments, n is an integer of 2. In some embodiments, n is an integer of 3. In some embodiments, n is an integer of 4.

In various embodiments, W is hydrogen. In various embodiments, W is unsubstituted methyl. In various embodiments, W is substituted methyl. In various embodiments, W is unsubstituted ethyl. In various embodiments, W is substituted ethyl. In various embodiments, W is unsubstituted alkyl. In various embodiments, W is substituted alkyl. In various embodiments, W is unsubstituted carbohydrate. In various embodiments, W is substituted carbohydrate. In various embodiments, W is potassium. In various embodiments, W is sodium. In various embodiments, W is lithium.

In various embodiments, Y is hydrogen. In various embodiments, Y is unsubstituted methyl. In various embodiments, Y is substituted methyl. In various embodiments, Y is unsubstituted ethyl. In various embodiments, Y is substituted ethyl. In various embodiments, Y is unsubstituted alkyl. In various embodiments, Y is substituted alkyl. In various embodiments, Y is unsubstituted carbohydrate. In various embodiments, Y is substituted carbohydrate. In various embodiments, Y is potassium. In various embodiments, Y is sodium. In various embodiments, Y is lithium.

In various embodiments, Z is calcium. In various embodiments, Z is magnesium. In various embodiments, Z is iron.

In various embodiments of the invention, the pyrone analog is of Formula VII or a pharmaceutically/veterinarily acceptable salt thereof:

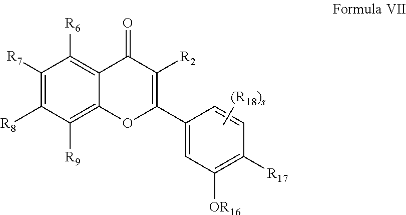

wherein R₂ is hydrogen, hydroxyl, C₁-C₁₀ alkyl, C₂-C₁₀ alkynyl, C₂-C₁₀ alkenyl, carboxyl, carbohydrate, ester, acyloxy, nitro, halogen, C₁-C₁₀ aliphatic acyl, C₆-C₁₀ aromatic acyl, C₆-C₁₀ aralkyl acyl, C₆-C₁₀ alkylaryl acyl, alkoxy, amine, aryl, C₄-C₁₀heterocyclyl, heteroaryl, C₃-C₁₀cycloalkyl, —OPO₃WY, —OCH₂PO₄WY, —OCH₂PO₄Z or —OPO₃Z;

R₆, R₇, R₈, and R₉ are independently hydrogen, hydroxyl, carboxaldehyde, amino, C₁-C₁₀ alkyl, C₂-C₁₀ alkynyl, C₂-C₁₀ alkenyl, carboxyl, carbohydrate, ester, acyloxy, nitro, halogen, C₁-C₁₀ aliphatic acyl, C₆-C₁₀ aromatic acyl, C₆-C₁₀ aralkyl acyl, C₆-C₁₀alkylaryl acyl, alkoxy, amine, aryl, C₃-C₁₀heterocyclyl, heteroaryl, C₃-C₁₀cycloalkyl, —OPO₃WY, —OCH₂PO₄WY, —OCH₂PO₄Z or —OPO₃Z;

R₁₆ is hydrogen, C₁-C₁₀ alkyl, C₂-C₁₀ alkynyl, C₂-C₁₀ alkenyl, carbohydrate C₁-C₁₀ aliphatic acyl, C₆-C₁₀ aromatic acyl, C₆-C₁₀ aralkyl acyl, C₆-C₁₀alkylaryl acyl, aryl, C₃-C₁₀heterocyclyl, heteroaryl, C₃-C₁₀cycloalkyl, —PO₃WY, —CH₂PO₄WY, —CH₂PO₄Z or —PO₃Z;

R₁₇ is hydrogen, hydroxy, carboxaldehyde, amine, C₁-C₁₀ alkyl, C₂-C₁₀ alkynyl, C₂-C₁₀ alkenyl, carboxyl, carbohydrate, ester, acyloxy, nitro, halogen, C₁-C₁₀ aliphatic acyl, C₆-C₁₀ aromatic acyl, C₆-C₁₀ aralkyl acyl, C₆-C₁₀ alkylaryl acyl, alkoxy, aryl, C₃-C₁₀ heterocyclyl, heteroaryl, or C₃-C₁₀cycloalkyl, —OPO₃WY, —OCH₂PO₄WY, —OCH₂PO₄Z or —OPO₃Z;

each instance of R₁₈ is independently hydrogen, hydroxyl, carboxaldehyde, amine, C₁-C₁₀ alkyl, C₂-C₁₀ alkynyl, C₂-C₁₀ alkenyl, carboxyl, carbohydrate, ester, acyloxy, nitro, halogen, C₁-C₁₀ aliphatic acyl, C₆-C₁₀ aromatic acyl, C₆-C₁₀ aralkyl acyl, C₆-C₁₀alkylaryl acyl, alkoxy, alkyl, phosphate, aryl, heteroaryl, C₃-C₁₀ heterocyclic, C₃-C₁₀cycloalkyl, —OPO₃WY, —OCH₂PO₄WY, —OCH₂PO₄Z or —OPO₃Z;

s is an integer of 0, 1, 2, or 3;

W and Y are independently hydrogen, methyl, ethyl, alkyl, carbohydrate, or a cation, and Z is a multivalent cation; and

wherein the compound of Formula VII is not the compound of Formula A:

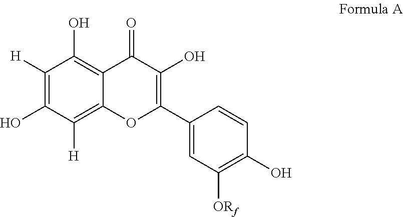

wherein R_{f} is hydrogen; and

wherein the compound of Formula VII is not the compound wherein:

R₂ is hydrogen, R₆, R₇, R₈, and R₉ are hydrogen, R₁₆ is hydrogen, R₁₇ is hydroxy, and s is 0;

R₂ is hydrogen, R₆, R₇, R₈, and R₉ are hydrogen, R₁₆ is hydrogen, R₁₇ is hydroxy, s is 1, and R₁₈ is 5′-hydroxy;

R₂ is hydrogen, R₆, R₇, R₈, and R₉ are hydrogen, R₁₆ is hydrogen, R₁₇ is hydroxy, s is 1, and R₁₈ is 5′-nitro;

R₂ is hydroxy, R₆, R₇, R₈, and R₉ are hydrogen, R₁₆ is hydrogen, R₁₇ is hydroxy, and s is 0;

R₂ is hydroxy, R₆, R₇, R₈, and R₉ are hydrogen, R₁₆ is hydrogen, R₁₇ is hydroxy, s is 1, and R₁₈ is 5′-hydroxy;

R₂ is hydrogen, R₆ and R₈ are hydroxy, R₇ and R₉ are hydrogen, R₁₆ is hydrogen, R₁₇ is hydroxy, and s is 0;

R₂ is hydrogen, R₆ and R₈ are hydroxy, R₇ and R₉ are hydrogen, R₁₆ is hydrogen, R₁₇ is hydroxy, s is 1, and R₁₈ is 5′-hydroxy;

R₂ is hydrogen, R₆ and R₈ are hydroxy, R₇ and R₉ are hydrogen, R₁₆ is hydrogen, R₁₇ is hydroxy, s is 1, and R₁₈ is 5′-methoxy;

R₂ is hydroxy, R₆ and R₈ are hydroxy, R₇ and R₉ are hydrogen, R₁₆ is methyl, R₁₇ is hydroxy, and s is 0;

R₂ is hydroxy, R₆ and R₈ are hydroxy, R₇ and R₉ are hydrogen, R₁₆ is hydrogen, R₁₇ is methoxy, and s is 0;

R₂ is hydrogen, R₆, R₈, and R₉ are hydrogen, R₇ is hydroxy, R₁₆ is hydrogen, R₁₇ is hydroxy, and s is 0;

R₂ is hydrogen, R₆, R₈, and R₉ are hydrogen, R₇ is hydroxy, R₁₆ is hydrogen, R₁₇ is hydroxy, s is 1, and R₁₈ is 5′-hydroxy;

R₂ is hydroxy, R₆, R₈, and R₉ are hydrogen, R₇ is hydroxy, R₁₆ is hydrogen, R₁₇ is hydroxy, and s is 0;

R₂ is hydrogen, R₆, R₇, and R₉ are hydrogen, R₈ is hydroxy, R₁₆ is hydrogen, R₁₇ is hydroxy, and s is 0;

R₂ is hydrogen, R₆, R₇, and R₉ are hydrogen, R₈ is hydroxy, R₁₆ is hydrogen, R₁₇ is hydroxy, s is 1, and R₁₈ is 5′-hydroxy;

R₂ is hydroxy, R₆, R₇, and R₉ are hydrogen, R₈ is hydroxy, R₁₆ is hydrogen, R₁₇ is hydroxy, and s is 0; or

R₂ is hydroxy, R₆, R₇, and R₉ are hydrogen, R₈ is hydroxy, R₁₆ is hydrogen, R₁₇ is hydroxy, s is 1, and R₁₈ is 5′-hydroxy.

In some embodiments, the compound of Formula VII is not the compound of Formula A wherein R_{f} is PO₃K₂.

In some embodiments of the invention, at least one of R₁₆ and R₁₉ is —PO₃WY, —CH₂PO₄WY, —CH₂PO₄Z or —PO₃Z, or at least one of R₁₇ or R₁₈ is —OPO₃WY, —OCH₂PO₄WY, —OCH₂PO₄Z or —OPO₃Z. In some embodiments, at least one of R₁₆ and R₁₉ is —PO₃WY, —CH₂PO₄WY, —CH₂PO₄Z or —PO₃Z and at least one of R₁₇ or R₁₈ is —OPO₃WY, —OCH₂PO₄WY, —OCH₂PO₄Z or —OPO₃Z.

In some embodiments, R₂ is hydrogen. In some embodiments, R₂ is hydroxyl. In some embodiments, R₂ is optionally substituted C₁-C₁₀ alkyl. In some embodiments, R₂ is unsubstituted C₁-C₁₀ alkyl. In some embodiments, R₂ is substituted C₁-C₁₀ alkyl. In some embodiments, R₂ is unsubstituted C₁-C₁₀ alkyl. In some other embodiments, R₂ is substituted C₁-C₁₀ alkyl. In some embodiments, R₂ is unsubstituted C₂-C₁₀ alkynyl. In some embodiments, R₂ is substituted C₂-C₁₀ alkynyl. In some embodiments, R₂ is unsubstituted C₂-C₁₀ alkenyl. In some embodiments, R₂ is substituted C₂-C₁₀ alkenyl. In some embodiments, R₂ is carboxyl. In some embodiments, R₂ is unsubstituted carbohydrate. In some embodiments, R₂ is substituted carbohydrate. In some embodiments, R₂ is unsubstituted ester. In some embodiments, R₂ is substituted ester. In some embodiments, R₂ is unsubstituted acyloxy. In some embodiments, R₂ is substituted acyloxy. In some embodiments, R₂ is nitro. In some embodiments, R₂ is halogen. In some embodiments, R₂ is unsubstituted C₁-C₁₀ aliphatic acyl. In some embodiments, R₂ is substituted C₁-C₁₀ aliphatic acyl. In some embodiments, R₂ is unsubstituted C₆-C₁₀ aromatic acyl. In some embodiments, R₂ is substituted C₆-C₁₀ aromatic acyl. In some embodiments, R₂ is unsubstituted C₆-C₁₀ aralkyl acyl. In some embodiments, R₂ is substituted C₆-C₁₀ aralkyl acyl. In some embodiments, R₂ is unsubstituted C₆-C₁₀ alkylaryl acyl. In some embodiments, R₂ is substituted C₆-C₁₀ alkylaryl acyl. In some embodiments, R₂ is unsubstituted alkoxy. In some embodiments, R₂ is substituted alkoxy. In some embodiments, R₂ is unsubstituted amine. In some embodiments, R₂ is substituted amine. In some embodiments, R₂ is unsubstituted aryl. In some embodiments, R₂ is substituted aryl. In some embodiments, R₂ is unsubstituted C₄-C₁₀heterocyclyl. In some embodiments, R₂ is substituted C₄-C₁₀heterocyclyl. In some embodiments, R₂ is unsubstituted heteroaryl. In some embodiments, R₂ is substituted heteroaryl. In some embodiments, R₂ is unsubstituted C₃-C₁₀cycloalkyl. In some embodiments, R₂ is substituted C₃-C₁₀cycloalkyl. In some embodiments, R₂ is —OPO₃WY. In some embodiments, R₂ is —OCH₂PO₄WY. In some embodiments, R₂ is —OCH₂PO₄Z. In some embodiments, R₂ is —OPO₃Z.

In some embodiments, R₆ is hydrogen. In some embodiments, R₆ is hydroxyl. In some embodiments, R₆ is carboxaldehyde. In some embodiments, R₆ is unsubstituted amine. In some embodiments, R₆ is substituted amine. In some embodiments, R₆ is unsubstituted C₁-C₁₀ alkyl. In some embodiments, R₆ is substituted C₁-C₁₀ alkyl. In some embodiments, R₆ is unsubstituted C₂-C₁₀ alkynyl. In some embodiments, R₆ is substituted C₂-C₁₀ alkynyl. In some embodiments, R₆ is unsubstituted C₂-C₁₀ alkenyl. In some embodiments, R₆ is substituted C₂-C₁₀ alkenyl. In some embodiments, R₆ is carboxyl. In some embodiments, R₆ is unsubstituted carbohydrate. In some embodiments, R₆ is substituted carbohydrate. In some embodiments, R₆ is unsubstituted ester. In some embodiments, R₆ is substituted ester. In some embodiments, R₆ is unsubstituted acyloxy. In some embodiments, R₆ is substituted acyloxy. In some embodiments, R₆ is nitro. In some embodiments, R₆ is halogen. In some embodiments, R₆ is unsubstituted C₁-C₁₀ aliphatic acyl. In some embodiments, R₆ is substituted C₁-C₁₀ aliphatic acyl. In some embodiments, R₆ is unsubstituted C₆-C₁₀ aromatic acyl. In some embodiments, R₆ is substituted C₆-C₁₀ aromatic acyl. In some embodiments, R₆ is unsubstituted C₆-C₁₀ aralkyl acyl. In some embodiments, R₆ is substituted C₆-C₁₀ aralkyl acyl. In some embodiments, R₆ is unsubstituted C₆-C₁₀ alkylaryl acyl. In some embodiments, R₆ is substituted C₆-C₁₀ alkylaryl acyl. In some embodiments, R₆ is unsubstituted alkoxy. In some embodiments, R₆ is substituted alkoxy. In some embodiments, R₆ is unsubstituted aryl. In some embodiments, R₆ is substituted aryl. In some embodiments, R₆ is unsubstituted C₃-C₁₀heterocyclyl. In some embodiments, R₆ is substituted C₃-C₁₀ heterocyclyl. In some embodiments, R₆ is unsubstituted heteroaryl, In some embodiments, R₆ is unsubstituted C₃-C₁₀cycloalkyl. In some embodiments, R₆ is substituted C₃-C₁₀cycloalkyl. In some embodiments, R₆ is —OPO₃WY. In some embodiments, R₆ is —OCH₂PO₄WY. In some embodiments, R₆ is —OCH₂PO₄Z. In some embodiments, R₆ is —OPO₃Z.

In some embodiments, R₇ is hydrogen. In some embodiments, R₇ is hydroxyl. In some embodiments, R₇ is carboxaldehyde. In some embodiments, R₇ is unsubstituted amine. In some embodiments, R₇ is substituted amine. In some embodiments, R₇ is unsubstituted C₁-C₁₀ alkyl. In some embodiments, R₇ is substituted C₁-C₁₀ alkyl. In some embodiments, R₇ is unsubstituted C₂-C₁₀ alkynyl. In some embodiments, R₇ is substituted C₂-C₁₀ alkynyl. In some embodiments, R₇ is unsubstituted C₂-C₁₀ alkenyl. In some embodiments, R₇ is substituted C₂-C₁₀ alkenyl. In some embodiments, R₇ is carboxyl. In some embodiments, R₇ is unsubstituted carbohydrate. In some embodiments, R₇ is substituted carbohydrate. In some embodiments, R₇ is unsubstituted ester. In some embodiments, R₇ is substituted ester. In some embodiments, R₇ is unsubstituted acyloxy. In some embodiments, R₇ is substituted acyloxy. In some embodiments, R₇ is nitro. In some embodiments, R₇ is halogen. In some embodiments, R₇ is unsubstituted C₁-C₁₀ aliphatic acyl. In some embodiments, R₇ is substituted C₁-C₁₀ aliphatic acyl. In some embodiments, R₇ is unsubstituted C₆-C₁₀ aromatic acyl. In some embodiments, R₇ is substituted C₆-C₁₀ aromatic acyl. In some embodiments, R₇ is unsubstituted C₆-C₁₀ aralkyl acyl. In some embodiments, R₇ is substituted C₆-C₁₀ aralkyl acyl. In some embodiments, R₇ is unsubstituted C₆-C₁₀ alkylaryl acyl. In some embodiments, R₇ is substituted C₆-C₁₀ alkylaryl acyl. In some embodiments, R₇ is unsubstituted alkoxy. In some embodiments, R₇ is substituted alkoxy. In some embodiments, R₇ is unsubstituted aryl. In some embodiments, R₇ is substituted aryl. In some embodiments, R₇ is unsubstituted C₃-C₁₀heterocyclyl. In some embodiments, R₇ is substituted C₃-C₁₀ heterocyclyl. In some embodiments, R₇ is unsubstituted heteroaryl. In some embodiments, R₇ is unsubstituted C₃-C₁₀cycloalkyl. In some embodiments, R₇ is substituted C₃-C₁₀cycloalkyl. In some embodiments, R₇ is —OPO₃WY. In some embodiments, R₇ is —OCH₂PO₄WY. In some embodiments, R₇ is —OCH₂PO₄Z. In some embodiments, R₇ is —OPO₃Z.

In some embodiments, R₈ is hydrogen. In some embodiments, R₈ is hydroxyl. In some embodiments, R₈ is carboxaldehyde. In some embodiments, R₈ is unsubstituted amine. In some embodiments, R₈ is substituted amine. In some embodiments, R₈ is unsubstituted C₁-C₁₀ alkyl. In some embodiments, R₈ is substituted C₁-C₁₀ alkyl. In some embodiments, R₈ is unsubstituted C₂-C₁₀ alkynyl. In some embodiments, R₈ is substituted C₂-C₁₀ alkynyl. In some embodiments, R₈ is unsubstituted C₂-C₁₀ alkenyl. In some embodiments, R₈ is substituted C₂-C₁₀ alkenyl. In some embodiments, R₈ is carboxyl. In some embodiments, R₈ is unsubstituted carbohydrate. In some embodiments, R₈ is substituted carbohydrate. In some embodiments, R₈ is unsubstituted ester. In some embodiments, R₈ is substituted ester. In some embodiments, R₈ is unsubstituted acyloxy. In some embodiments, R₈ is substituted acyloxy. In some embodiments, R₈ is nitro. In some embodiments, R₈ is halogen. In some embodiments, R₈ is unsubstituted C₁-C₁₀ aliphatic acyl. In some embodiments, R₈ is substituted C₁-C₁₀ aliphatic acyl. In some embodiments, R₈ is unsubstituted C₆-C₁₀ aromatic acyl. In some embodiments, R₈ is substituted C₆-C₁₀ aromatic acyl. In some embodiments, R₈ is unsubstituted C₆-C₁₀ aralkyl acyl. In some embodiments, R₈ is substituted C₆-C₁₀ aralkyl acyl. In some embodiments, R₈ is unsubstituted C₆-C₁₀ alkylaryl acyl. In some embodiments, R₈ is substituted C₆-C₁₀ alkylaryl acyl. In some embodiments, R₈ is unsubstituted alkoxy. In some embodiments, R₈ is substituted alkoxy. In some embodiments, R₈ is unsubstituted aryl. In some embodiments, R₈ is substituted aryl. In some embodiments, R₈ is unsubstituted C₃-C₁₀heterocyclyl. In some embodiments, R₈ is substituted C₃-C₁₀ heterocyclyl. In some embodiments, R₈ is unsubstituted heteroaryl, In some embodiments, R₈ is unsubstituted C₃-C₁₀cycloalkyl. In some embodiments, R₈ is substituted C₃-C₁₀cycloalkyl. In some embodiments, R₈ is —OPO₃WY. In some embodiments, R₈ is —OCH₂PO₄WY. In some embodiments, R₈ is —OCH₂PO₄Z. In some embodiments, R₈ is —OPO₃Z.

In some embodiments, R₉ is hydrogen. In some embodiments, R₉ is hydroxyl. In some embodiments, R₉ is carboxaldehyde. In some embodiments, R₉ is unsubstituted amine. In some embodiments, R₉ is substituted amine. In some embodiments, R₉ is unsubstituted C₁-C₁₀ alkyl. In some embodiments, R₉ is substituted C₁-C₁₀ alkyl. In some embodiments, R₉ is unsubstituted C₂-C₁₀ alkynyl. In some embodiments, R₉ is substituted C₂-C₁₀ alkynyl. In some embodiments, R₉ is unsubstituted C₂-C₁₀ alkenyl. In some embodiments, R₉ is substituted C₂-C₁₀ alkenyl. In some embodiments, R₉ is carboxyl. In some embodiments, R₉ is unsubstituted carbohydrate. In some embodiments, R₉ is substituted carbohydrate. In some embodiments, R₉ is unsubstituted ester. In some embodiments, R₉ is substituted ester. In some embodiments, R₉ is unsubstituted acyloxy. In some embodiments, R₉ is substituted acyloxy. In some embodiments, R₉ is nitro. In some embodiments, R₉ is halogen. In some embodiments, R₉ is unsubstituted C₁-C₁₀ aliphatic acyl. In some embodiments, R₉ is substituted C₁-C₁₀ aliphatic acyl. In some embodiments, R₉ is unsubstituted C₆-C₁₀ aromatic acyl. In some embodiments, R₉ is substituted C₆-C₁₀ aromatic acyl. In some embodiments, R₉ is unsubstituted C₆-C₁₀ aralkyl acyl. In some embodiments, R₉ is substituted C₆-C₁₀ aralkyl acyl. In some embodiments, R₉ is unsubstituted C₆-C₁₀ alkylaryl acyl. In some embodiments, R₉ is substituted C₆-C₁₀ alkylaryl acyl. In some embodiments, R₉ is unsubstituted alkoxy. In some embodiments, R₉ is substituted alkoxy. In some embodiments, R₉ is unsubstituted aryl. In some embodiments, R₉ is substituted aryl. In some embodiments, R₉ is unsubstituted C₃-C₁₀heterocyclyl. In some embodiments, R₉ is substituted C₃-C₁₀ heterocyclyl. In some embodiments, R₉ is unsubstituted heteroaryl, In some embodiments, R₉ is unsubstituted C₃-C₁₀cycloalkyl. In some embodiments, R₉ is substituted C₃-C₁₀cycloalkyl. In some embodiments, R₉ is —OPO₃WY. In some embodiments, R₉ is —OCH₂PO₄WY. In some embodiments, R₉ is —OCH₂PO₄Z. In some embodiments, R₉ is —OPO₃Z.

In some embodiments, R₁₆ is hydrogen. In some embodiments, R₁₆ is unsubstituted C₁-C₁₀ alkyl. In some embodiments, R₁₆ is substituted C₁-C₁₀ alkyl. In some embodiments, R₁₆ is unsubstituted C₂-C₁₀ alkynyl. In some embodiments, R₁₆ is substituted C₂-C₁₀ alkynyl. In some embodiments, R₁₆ is unsubstituted C₂-C₁₀ alkenyl. In some embodiments, R₁₆ is substituted C₂-C₁₀ alkenyl. In some embodiments, R₁₆ is unsubstituted carbohydrate. In some embodiments, R₁₆ is substituted carbohydrate. In some embodiments, R₁₆ is unsubstituted C₁-C₁₀ aliphatic acyl. In some embodiments, R₁₆ is substituted C₁-C₁₀ aliphatic acyl. In some embodiments, R₁₆ is unsubstituted C₆-C₁₀ aromatic acyl. In some embodiments, R₁₆ is substituted C₆-C₁₀ aromatic acyl. In some embodiments, R₁₆ is unsubstituted C₆-C₁₀ aralkyl acyl. In some embodiments, R₁₆ is substituted C₆-C₁₀ aralkyl acyl. In some embodiments, R₁₆ is unsubstituted C₆-C₁₀ alkylaryl acyl. In some embodiments, R₁₆ is substituted C₆-C₁₀ alkylaryl acyl. In some embodiments, R₁₆ is unsubstituted aryl. In some embodiments, R₁₆ is substituted aryl. In some embodiments, R₁₆ is unsubstituted C₃-C₁₀heterocyclyl. In some embodiments, R₁₆ is substituted C₃-C₁₀ heterocyclyl. In some embodiments, R₁₆ is unsubstituted heteroaryl. In some embodiments, R₁₆ is substituted heteroaryl. In some embodiments, R₁₆ is unsubstituted C₃-C₁₀cycloalkyl. In some embodiments, R₁₆ is substituted C₃-C₁₀cycloalkyl. In some embodiments, R₁₆ is —PO₃WY. In some embodiments, R₁₆ is —CH₂PO₄WY. In some embodiments, R₁₆ is —CH₂PO₄Z. In some embodiments, R₁₆ is —PO₃Z.

In some embodiments, R₁₇ is hydrogen. In some embodiments, R₁₇ is hydroxy. In some embodiments, R₁₇ is carboxaldehyde. In some embodiments, R₁₇ is unsubstituted amine. In some embodiments, R₁₇ is substituted amine. In some embodiments, R₁₇ is unsubstituted C₁-C₁₀ alkyl. In some embodiments, R₁₇ is unsubstituted C₂-C₁₀ alkynyl. In some embodiments, R₁₇ is substituted C₂-C₁₀ alkynyl. In some embodiments, R₁₇ is unsubstituted C₂-C₁₀ alkenyl. In some embodiments, R₁₇ is substituted C₂-C₁₀ alkenyl. In some embodiments, R₁₇ is carboxyl. In some embodiments, R₁₇ is unsubstituted carbohydrate. In some embodiments, R₁₇ is substituted carbohydrate. In some embodiments, R₁₇ is unsubstituted ester. In some embodiments, R₁₇ is substituted ester. In some embodiments, R₁₇ is unsubstituted acyloxy. In some embodiments, R₁₇ is substituted acyloxy. In some embodiments, R₁₇ is nitro. In some embodiments, R₁₇ is halogen. In some embodiments, R₁₇ is unsubstituted C₁-C₁₀ aliphatic acyl. In some embodiments, R₁₇ is substituted C₁-C₁₀ aliphatic acyl. In some embodiments, R₁₇ is unsubstituted C₆-C₁₀ aromatic acyl. In some embodiments, R₁₇ is substituted C₆-C₁₀ aromatic acyl. In some embodiments, R₁₇ is unsubstituted C₆-C₁₀ aralkyl acyl. In some embodiments, R₁₇ is substituted C₆-C₁₀ aralkyl acyl. In some embodiments, R₁₇ is unsubstituted C₆-C₁₀ alkylaryl acyl. n some embodiments, R₁₇ is substituted C₆-C₁₀ alkylaryl acyl. In some embodiments, R₁₇ is unsubstituted alkoxy. In some embodiments, R₁₇ is substituted alkoxy. In some embodiments, R₁₇ is unsubstituted aryl. In some embodiments, R₁₇ is substituted aryl. In some embodiments, R₁₇ is unsubstituted C₃-C₁₀heterocyclyl. In some embodiments, R₁₇ is substituted C₃-C₁₀heterocyclyl. In some embodiments, R₁₇ is unsubstituted heteroaryl. In some embodiments, R₁₇ is substituted heteroaryl. In some embodiments, R₁₇ is unsubstituted C₃-C₁₀cycloalkyl. In some embodiments, R₁₇ is substituted C₃-C₁₀cycloalkyl. In some embodiments, R₁₇ is —OPO₃WY. In some embodiments, R₁₇ is —OCH₂PO₄WY. In some embodiments, R₁₇ is —OCH₂PO₄Z. In some embodiments, R₁₇ is —OPO₃Z.

In some embodiments, R₁₈ is hydrogen. In some embodiments, R₁₈ is hydroxy. In some embodiments, R₁₈ is carboxaldehyde. In some embodiments, R₁₈ is unsubstituted amine. In some embodiments, R₁₈ is substituted amine. In some embodiments, R₁₈ is unsubstituted C₁-C₁₀ alkyl. In some embodiments, R₁₈ is unsubstituted C₂-C₁₀ alkynyl. In some embodiments, R₁₈ is substituted C₂-C₁₀ alkynyl. In some embodiments, R₁₈ is unsubstituted C₂-C₁₀ alkenyl. In some embodiments, R₁₈ is substituted C₂-C₁₀ alkenyl. In some embodiments, R₁₈ is carboxyl. In some embodiments, R₁₈ is unsubstituted carbohydrate. In some embodiments, R₁₈ is substituted carbohydrate. In some embodiments, R₁₈ is substituted carbohydrate. In some embodiments, R₁₈ is unsubstituted ester. In some embodiments, R₁₈ is substituted ester. In some embodiments, R₁₈ is unsubstituted acyloxy. In some embodiments, R₁₈ is substituted acyloxy. In some embodiments, R₁₈ is nitro. In some embodiments, R₁₈ is halogen. In some embodiments, R₁₈ is unsubstituted C₁-C₁₀ aliphatic acyl. In some embodiments, R₁₈ is substituted C₁-C₁₀ aliphatic acyl. In some embodiments, R₁₈ is unsubstituted C₆-C₁₀ aromatic acyl. In some embodiments, R₁₈ is substituted C₆-C₁₀ aromatic acyl. In some embodiments, R₁₈ is unsubstituted C₆-C₁₀ aralkyl acyl. In some embodiments, R₁₈ is substituted C₆-C₁₀ aralkyl acyl. In some embodiments, R₁₈ is unsubstituted C₆-C₁₀ alkylaryl acyl. In some embodiments, R₁₈ is substituted C₆-C₁₀ alkylaryl acyl. In some embodiments, R₁₈ is unsubstituted alkoxy. In some embodiments, R₁₈ is substituted alkoxy. In some embodiments, R₁₈ is unsubstituted aryl. In some embodiments, R₁₈ is substituted aryl. In some embodiments, R₁₈ is unsubstituted C₃-C₁₀heterocyclyl. In some embodiments, R₁₈ is substituted C₃-C₁₀heterocyclyl. In some embodiments, R₁₈ is unsubstituted heteroaryl. In some embodiments, R₁₈ is substituted heteroaryl. In some embodiments, R₁₈ is unsubstituted C₃-C₁₀cycloalkyl. In some embodiments, R₁₈ is substituted C₃-C₁₀cycloalkyl. In some embodiments, R₁₈ is —OPO₃WY. In some embodiments, R₁₈ is —OCH₂PO₄WY. In some embodiments, R₁₈ is —OCH₂PO₄Z. In some embodiments, R₁₈ is —OPO₃Z.

In some embodiments, s is an integer of 0. In some embodiments, s is an integer of 1. In some embodiments, s is an integer of 2. In some embodiments, s is an integer of 3.

In various embodiments, W is hydrogen. In various embodiments, W is unsubstituted methyl. In various embodiments, W is substituted methyl. In various embodiments, W is unsubstituted ethyl. In various embodiments, W is substituted ethyl. In various embodiments, W is unsubstituted alkyl. In various embodiments, W is substituted alkyl. In various embodiments, W is unsubstituted carbohydrate. In various embodiments, W is substituted carbohydrate. In various embodiments, W is potassium. In various embodiments, W is sodium. In various embodiments, W is lithium.

In various embodiments, Y is hydrogen. In various embodiments, Y is unsubstituted methyl. In various embodiments, Y is substituted methyl. In various embodiments, Y is unsubstituted ethyl. In various embodiments, Y is substituted ethyl. In various embodiments, Y is unsubstituted alkyl. In various embodiments, Y is substituted alkyl. In various embodiments, Y is unsubstituted carbohydrate. In various embodiments, Y is substituted carbohydrate. In various embodiments, Y is potassium. In various embodiments, Y is sodium. In various embodiments, Y is lithium.

In various embodiments, Z is calcium. In various embodiments, Z is magnesium. In various embodiments, Z is iron.

In other embodiments of the invention, the pyrone analog of the invention is a compound of Formula VIII or a pharmaceutically/veterinarily acceptable salt thereof:

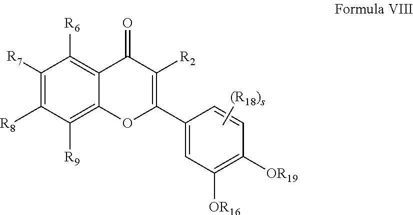

wherein R₂ is hydrogen, hydroxyl, C₁-C₁₀ alkyl, C₂-C₁₀ alkynyl, C₂-C₁₀ alkenyl, carboxyl, carbohydrate, ester, acyloxy, nitro, halogen, C₁-C₁₀ aliphatic acyl, C₆-C₁₀ aromatic acyl, C₆-C₁₀ aralkyl acyl, C₆-C₁₀ alkylaryl acyl, alkoxy, amine, aryl, C₄-C₁₀heterocyclyl, heteroaryl, C₃-C₁₀cycloalkyl, —OPO₃WY, —OCH₂PO₄WY, —OCH₂PO₄Z or —OPO₃Z;

R₆, R₇, R₈, and R₉ are independently hydrogen, hydroxyl, carboxaldehyde, amino, C₁-C₁₀ alkyl, C₂-C₁₀ alkynyl, C₂-C₁₀ alkenyl, carboxyl, carbohydrate, ester, acyloxy, nitro, halogen, C₁-C₁₀ aliphatic acyl, C₆-C₁₀ aromatic acyl, C₆-C₁₀ aralkyl acyl, C₆-C₁₀alkylaryl acyl, alkoxy, amine, aryl, C₃-C₁₀heterocyclyl, heteroaryl, C₃-C₁₀cycloalkyl, —OPO₃WY, —OCH₂PO₄WY, —OCH₂PO₄Z or —OPO₃Z;

wherein R₁₆ is hydrogen, C₁-C₁₀ alkyl, C₂-C₁₀ alkynyl, C₂-C₁₀ alkenyl, carbohydrate, C₁-C₁₀ aliphatic acyl, C₆-C₁₀ aromatic acyl, C₆-C₁₀ aralkyl acyl, C₆-C₁₀ alkylaryl acyl, aryl, C₃-C₁₀ heterocyclyl, heteroaryl, C₃-C₁₀cycloalkyl, —PO₃WY, —CH₂PO₄WY, —CH₂PO₄Z or —PO₃Z;

each instance of R₁₈ is independently hydrogen, hydroxyl, carboxaldehyde, amine, C₁-C₁₀ alkyl, C₂-C₁₀ alkynyl, C₂-C₁₀ alkenyl, carboxyl, carbohydrate, ester, acyloxy, nitro, halogen, C₁-C₁₀ aliphatic acyl, C₆-C₁₀ aromatic acyl, C₆-C₁₀ aralkyl acyl, C₆-C₁₀alkylaryl acyl, alkoxy, alkyl, phosphate, aryl, heteroaryl, C₃-C₁₀ heterocyclic, C₃-C₁₀cycloalkyl, —OPO₃WY, —OCH₂PO₄WY, —OCH₂PO₄Z or —OPO₃Z;

R₁₉ is hydrogen, C₁-C₁₀ alkyl, C₂-C₁₀ alkynyl, C₂-C₁₀ alkenyl, carbohydrate, C₁-C₁₀ aliphatic acyl, C₆-C₁₀ aromatic acyl, C₆-C₁₀ aralkyl acyl, C₆-C₁₀ alkylaryl acyl, aryl, C₃-C₁₀heterocyclyl, heteroaryl, optionally substituted C₃-C₁₀cycloalkyl, —PO₃WY, —CH₂PO₄WY, —CH₂PO₄Z or —PO₃Z;

s is an integer of 0, 1, 2, or 3; and

W and Y are independently hydrogen, methyl, ethyl, alkyl, carbohydrate, or a cation, and Z is a multivalent cation; and

wherein the compound of Formula VIII is not the compound of Formula A:

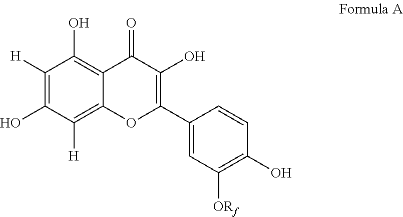

wherein R_{f} is hydrogen; and

wherein the compound of Formula VII is not the compound wherein:

R₂ is hydrogen, R₆, R₇, R₈, and R₉ are hydrogen, R₁₆ is hydrogen, R₁₇ is hydroxy, and s is 0;

R₂ is hydrogen, R₆, R₇, R₈, and R₉ are hydrogen, R₁₆ is hydrogen, R₁₇ is hydroxy, s is 1, and R₁₈ is 5′-hydroxy;

R₂ is hydrogen, R₆, R₇, R₈, and R₉ are hydrogen, R₁₆ is hydrogen, R₁₇ is hydroxy, s is 1, and R₁₈ is 5′-nitro;

R₂ is hydroxy, R₆, R₇, R₈, and R₉ are hydrogen, R₁₆ is hydrogen, R₁₇ is hydroxy, and s is 0;

R₂ is hydroxy, R₆, R₇, R₈, and R₉ are hydrogen, R₁₆ is hydrogen, R₁₇ is hydroxy, s is 1, and R₁₈ is 5′-hydroxy;

R₂ is hydrogen, R₆ and R₈ are hydroxy, R₇ and R₉ are hydrogen, R₁₆ is hydrogen, R₁₇ is hydroxy, and s is 0;

R₂ is hydrogen, R₆ and R₈ are hydroxy, R₇ and R₉ are hydrogen, R₁₆ is hydrogen, R₁₇ is hydroxy, s is 1, and

R₁₈ is 5′-hydroxy;

R₂ is hydrogen, R₆ and R₈ are hydroxy, R₇ and R₉ are hydrogen, R₁₆ is hydrogen, R₁₇ is hydroxy, s is 1, and

R₁₈ is 5′-methoxy;

R₂ is hydroxy, R₆ and R₈ are hydroxy, R₇ and R₉ are hydrogen, R₁₆ is methyl, R₁₇ is hydroxy, and s is 0;

R₂ is hydroxy, R₆ and R₈ are hydroxy, R₇ and R₉ are hydrogen, R₁₆ is hydrogen, R₁₇ is methoxy, and s is 0;

R₂ is hydrogen, R₆, R₈, and R₉ are hydrogen, R₇ is hydroxy, R₁₆ is hydrogen, R₁₇ is hydroxy, and s is 0;

R₂ is hydrogen, R₆, R₈, and R₉ are hydrogen, R₇ is hydroxy, R₁₆ is hydrogen, R₁₇ is hydroxy, s is 1, and R₁₈ is 5′-hydroxy;

R₂ is hydroxy, R₆, R₈, and R₉ are hydrogen, R₇ is hydroxy, R₁₆ is hydrogen, R₁₇ is hydroxy, and s is 0;

R₂ is hydrogen, R₆, R₇, and R₉ are hydrogen, R₈ is hydroxy, R₁₆ is hydrogen, R₁₇ is hydroxy, and s is 0;

R₂ is hydrogen, R₆, R₇, and R₉ are hydrogen, R₈ is hydroxy, R₁₆ is hydrogen, R₁₇ is hydroxy, s is 1, and R₁₈ is 5′-hydroxy;

R₂ is hydroxy, R₆, R₇, and R₉ are hydrogen, R₈ is hydroxy, R₁₆ is hydrogen, R₁₇ is hydroxy, and s is 0; or

R₂ is hydroxy, R₆, R₇, and R₉ are hydrogen, R₈ is hydroxy, R₁₆ is hydrogen, R₁₇ is hydroxy, s is 1, and R₁₈ is 5′-hydroxy.

In some embodiments, the compound of Formula VIII is not the compound of Formula A wherein R_{f} is PO₃K₂.

In some embodiments of the invention, at least one of R₁₆ and R₁₉ is —PO₃WY, —CH₂PO₄WY, —CH₂PO₄Z or —PO₃Z, or at least one of R₁₇ or R₁₈ is —OPO₃WY, —OCH₂PO₄WY, —OCH₂PO₄Z or —OPO₃Z. In some embodiments, at least one of R₁₆ and R₁₉ is —PO₃WY, —CH₂PO₄WY, —CH₂PO₄Z or —PO₃Z and at least one of R₁₇ or R₁₈ is —OPO₃WY, —OCH₂PO₄WY, —OCH₂PO₄Z or —OPO₃Z.

In some embodiments, R₂ is hydrogen. In some embodiments, R₂ is hydroxyl. In some embodiments, R₂ is optionally substituted C₁-C₁₀ alkyl. In some embodiments, R₂ is unsubstituted C₁-C₁₀ alkyl. In some embodiments, R₂ is substituted C₁-C₁₀ alkyl. In some embodiments, R₂ is unsubstituted C₁-C₁₀ alkyl. In some other embodiments, R₂ is substituted C₁-C₁₀ alkyl. In some embodiments, R₂ is unsubstituted C₂-C₁₀ alkynyl. In some embodiments, R₂ is substituted C₂-C₁₀ alkynyl. In some embodiments, R₂ is unsubstituted C₂-C₁₀ alkenyl. In some embodiments, R₂ is substituted C₂-C₁₀ alkenyl. In some embodiments, R₂ is carboxyl. In some embodiments, R₂ is unsubstituted carbohydrate. In some embodiments, R₂ is substituted carbohydrate. In some embodiments, R₂ is unsubstituted ester. In some embodiments, R₂ is substituted ester. In some embodiments, R₂ is unsubstituted acyloxy. In some embodiments, R₂ is substituted acyloxy. In some embodiments, R₂ is nitro. In some embodiments, R₂ is halogen. In some embodiments, R₂ is unsubstituted C₁-C₁₀ aliphatic acyl. In some embodiments, R₂ is substituted C₁-C₁₀ aliphatic acyl. In some embodiments, R₂ is unsubstituted C₆-C₁₀ aromatic acyl. In some embodiments, R₂ is substituted C₆-C₁₀ aromatic acyl. In some embodiments, R₂ is unsubstituted C₆-C₁₀ aralkyl acyl. In some embodiments, R₂ is substituted C₆-C₁₀ aralkyl acyl. In some embodiments, R₂ is unsubstituted C₆-C₁₀ alkylaryl acyl. In some embodiments, R₂ is substituted C₆-C₁₀ alkylaryl acyl. In some embodiments, R₂ is unsubstituted alkoxy. In some embodiments, R₂ is substituted alkoxy. In some embodiments, R₂ is unsubstituted amine. In some embodiments, R₂ is substituted amine. In some embodiments, R₂ is unsubstituted aryl. In some embodiments, R₂ is substituted aryl. In some embodiments, R₂ is unsubstituted C₄-C₁₀heterocyclyl. In some embodiments, R₂ is substituted C₄-C₁₀heterocyclyl. In some embodiments, R₂ is unsubstituted heteroaryl. In some embodiments, R₂ is substituted heteroaryl. In some embodiments, R₂ is unsubstituted C₃-C₁₀cycloalkyl. In some embodiments, R₂ is substituted C₃-C₁₀cycloalkyl. In some embodiments, R₂ is —OPO₃WY. In some embodiments, R₂ is —OCH₂PO₄WY.

In some embodiments, R₂ is —OCH₂PO₄Z. In some embodiments, R₂ is —OPO₃Z. In some embodiments, R₆ is hydrogen. In some embodiments, R₆ is hydroxyl. In some embodiments, R₆ is carboxaldehyde. In some embodiments, R₆ is unsubstituted amine. In some embodiments, R₆ is substituted amine. In some embodiments, R₆ is unsubstituted C₁-C₁₀ alkyl. In some embodiments, R₆ is substituted C₁-C₁₀ alkyl. In some embodiments, R₆ is unsubstituted C₂-C₁₀ alkynyl. In some embodiments, R₆ is substituted C₂-C₁₀ alkynyl. In some embodiments, R₆ is unsubstituted C₂-C₁₀ alkenyl. In some embodiments, R₆ is substituted C₂-C₁₀ alkenyl. In some embodiments, R₆ is carboxyl. In some embodiments, R₆ is unsubstituted carbohydrate. In some embodiments, R₆ is substituted carbohydrate. In some embodiments, R₆ is unsubstituted ester. In some embodiments, R₆ is substituted ester. In some embodiments, R₆ is unsubstituted acyloxy. In some embodiments, R₆ is substituted acyloxy. In some embodiments, R₆ is nitro. In some embodiments, R₆ is halogen. In some embodiments, R₆ is unsubstituted C₁-C₁₀ aliphatic acyl. In some embodiments, R₆ is substituted C₁-C₁₀ aliphatic acyl. In some embodiments, R₆ is unsubstituted C₆-C₁₀ aromatic acyl. In some embodiments, R₆ is substituted C₆-C₁₀ aromatic acyl. In some embodiments, R₆ is unsubstituted C₆-C₁₀ aralkyl acyl. In some embodiments, R₆ is substituted C₆-C₁₀ aralkyl acyl. In some embodiments, R₆ is unsubstituted C₆-C₁₀ alkylaryl acyl. In some embodiments, R₆ is substituted C₆-C₁₀ alkylaryl acyl. In some embodiments, R₆ is unsubstituted alkoxy. In some embodiments, R₆ is substituted alkoxy. In some embodiments, R₆ is unsubstituted aryl. In some embodiments, R₆ is substituted aryl. In some embodiments, R₆ is unsubstituted C₃-C₁₀heterocyclyl. In some embodiments, R₆ is substituted C₃-C₁₀ heterocyclyl. In some embodiments, R₆ is unsubstituted heteroaryl, In some embodiments, R₆ is unsubstituted C₃-C₁₀cycloalkyl. In some embodiments, R₆ is substituted C₃-C₁₀cycloalkyl. In some embodiments, R₆ is —OPO₃WY. In some embodiments, R₆ is —OCH₂PO₄WY. In some embodiments, R₆ is —OCH₂PO₄Z. In some embodiments, R₆ is —OPO₃Z.

In some embodiments, R₇ is hydrogen. In some embodiments, R₇ is hydroxyl. In some embodiments, R₇ is carboxaldehyde. In some embodiments, R₇ is unsubstituted amine. In some embodiments, R₇ is substituted amine. In some embodiments, R₇ is unsubstituted C₁-C₁₀ alkyl. In some embodiments, R₇ is substituted C₁-C₁₀ alkyl. In some embodiments, R₇ is unsubstituted C₂-C₁₀ alkynyl. In some embodiments, R₇ is substituted C₂-C₁₀ alkynyl. In some embodiments, R₇ is unsubstituted C₂-C₁₀ alkenyl. In some embodiments, R₇ is substituted C₂-C₁₀ alkenyl. In some embodiments, R₇ is carboxyl. In some embodiments, R₇ is unsubstituted carbohydrate. In some embodiments, R₇ is substituted carbohydrate. In some embodiments, R₇ is unsubstituted ester. In some embodiments, R₇ is substituted ester. In some embodiments, R₇ is unsubstituted acyloxy. In some embodiments, R₇ is substituted acyloxy. In some embodiments, R₇ is nitro. In some embodiments, R₇ is halogen. In some embodiments, R₇ is unsubstituted C₁-C₁₀ aliphatic acyl. In some embodiments, R₇ is substituted C₁-C₁₀ aliphatic acyl. In some embodiments, R₇ is unsubstituted C₆-C₁₀ aromatic acyl. In some embodiments, R₇ is substituted C₆-C₁₀ aromatic acyl. In some embodiments, R₇ is unsubstituted C₆-C₁₀ aralkyl acyl. In some embodiments, R₇ is substituted C₆-C₁₀ aralkyl acyl. In some embodiments, R₇ is unsubstituted C₆-C₁₀ alkylaryl acyl. In some embodiments, R₇ is substituted C₆-C₁₀ alkylaryl acyl. In some embodiments, R₇ is unsubstituted alkoxy. In some embodiments, R₇ is substituted alkoxy. In some embodiments, R₇ is unsubstituted aryl. In some embodiments, R₇ is substituted aryl. In some embodiments, R₇ is unsubstituted C₃-C₁₀heterocyclyl. In some embodiments, R₇ is substituted C₃-C₁₀ heterocyclyl. In some embodiments, R₇ is unsubstituted heteroaryl, In some embodiments, R₇ is unsubstituted C₃-C₁₀cycloalkyl. In some embodiments, R₇ is substituted C₃-C₁₀cycloalkyl. In some embodiments, R₇ is —OPO₃WY. In some embodiments, R₇ is —OCH₂PO₄WY. In some embodiments, R₇ is —OCH₂PO₄Z. In some embodiments, R₇ is —OPO₃Z.

In some embodiments, R₈ is hydrogen. In some embodiments, R₈ is hydroxyl. In some embodiments, R₈ is carboxaldehyde. In some embodiments, R₈ is unsubstituted amine. In some embodiments, R₈ is substituted amine. In some embodiments, R₈ is unsubstituted C₁-C₁₀ alkyl. In some embodiments, R₈ is substituted C₁-C₁₀ alkyl. In some embodiments, R₈ is unsubstituted C₂-C₁₀ alkynyl. In some embodiments, R₈ is substituted C₂-C₁₀ alkynyl. In some embodiments, R₈ is unsubstituted C₂-C₁₀ alkenyl. In some embodiments, R₈ is substituted C₂-C₁₀ alkenyl. In some embodiments, R₈ is carboxyl. In some embodiments, R₈ is unsubstituted carbohydrate. In some embodiments, R₈ is substituted carbohydrate. In some embodiments, R₈ is unsubstituted ester. In some embodiments, R₈ is substituted ester. In some embodiments, R₈ is unsubstituted acyloxy. In some embodiments, R₈ is substituted acyloxy. In some embodiments, R₈ is nitro. In some embodiments, R₈ is halogen. In some embodiments, R₈ is unsubstituted C₁-C₁₀ aliphatic acyl. In some embodiments, R₈ is substituted C₁-C₁₀ aliphatic acyl. In some embodiments, R₈ is unsubstituted C₆-C₁₀ aromatic acyl. In some embodiments, R₈ is substituted C₆-C₁₀ aromatic acyl. In some embodiments, R₈ is unsubstituted C₆-C₁₀ aralkyl acyl. In some embodiments, R₈ is substituted C₆-C₁₀ aralkyl acyl. In some embodiments, R₈ is unsubstituted C₆-C₁₀ alkylaryl acyl. In some embodiments, R₈ is substituted C₆-C₁₀ alkylaryl acyl. In some embodiments, R₈ is unsubstituted alkoxy. In some embodiments, R₈ is substituted alkoxy. In some embodiments, R₈ is unsubstituted aryl. In some embodiments, R₈ is substituted aryl. In some embodiments, R₈ is unsubstituted C₃-C₁₀heterocyclyl. In some embodiments, R₈ is substituted C₃-C₁₀ heterocyclyl. In some embodiments, R₈ is unsubstituted heteroaryl, In some embodiments, R₈ is unsubstituted C₃-C₁₀cycloalkyl. In some embodiments, R₈ is substituted C₃-C₁₀cycloalkyl. In some embodiments, R₈ is —OPO₃WY. In some embodiments, R₈ is —OCH₂PO₄WY. In some embodiments, R₈ is —OCH₂PO₄Z. In some embodiments, R₈ is —OPO₃Z.

In some embodiments, R₉ is hydrogen. In some embodiments, R₉ is hydroxyl. In some embodiments, R₉ is carboxaldehyde. In some embodiments, R₉ is unsubstituted amine. In some embodiments, R₉ is substituted amine. In some embodiments, R₉ is unsubstituted C₁-C₁₀ alkyl. In some embodiments, R₉ is substituted C₁-C₁₀ alkyl. In some embodiments, R₉ is unsubstituted C₂-C₁₀ alkynyl. In some embodiments, R₉ is substituted C₂-C₁₀ alkynyl. In some embodiments, R₉ is unsubstituted C₂-C₁₀ alkenyl. In some embodiments, R₉ is substituted C₂-C₁₀ alkenyl. In some embodiments, R₉ is carboxyl. In some embodiments, R₉ is unsubstituted carbohydrate. In some embodiments, R₉ is substituted carbohydrate. In some embodiments, R₉ is unsubstituted ester. In some embodiments, R₉ is substituted ester. In some embodiments, R₉ is unsubstituted acyloxy. In some embodiments, R₉ is substituted acyloxy. In some embodiments, R₉ is nitro. In some embodiments, R₉ is halogen. In some embodiments, R₉ is unsubstituted C₁-C₁₀ aliphatic acyl. In some embodiments, R₉ is substituted C₁-C₁₀ aliphatic acyl. In some embodiments, R₉ is unsubstituted C₆-C₁₁ aromatic acyl. In some embodiments, R₉ is substituted C₆-C₁ aromatic acyl. In some embodiments, R₉ is unsubstituted C₆-C₁₁ aralkyl acyl. In some embodiments, R₉ is substituted C₆-C₁₀ aralkyl acyl. In some embodiments, R₉ is unsubstituted C₆-C₁₀ alkylaryl acyl. In some embodiments, R₉ is substituted C₆-C₁₀ alkylaryl acyl. In some embodiments, R₉ is unsubstituted alkoxy. In some embodiments, R₉ is substituted alkoxy. In some embodiments, R₉ is unsubstituted aryl. In some embodiments, R₉ is substituted aryl. In some embodiments, R₉ is unsubstituted C₃-C₁₀heterocyclyl. In some embodiments, R₉ is substituted C₃-C₁₀ heterocyclyl. In some embodiments, R₉ is unsubstituted heteroaryl, In some embodiments, R₉ is unsubstituted C₃-C₁₀cycloalkyl. In some embodiments, R₉ is substituted C₃-C₁₀cycloalkyl. In some embodiments, R₉ is —OPO₃WY. In some embodiments, R₉ is —OCH₂PO₄WY. In some embodiments, R₉ is —OCH₂PO₄Z. In some embodiments, R₉ is —OPO₃Z.

In some embodiments, R₁₆ is hydrogen. In some embodiments, R₁₆ is unsubstituted C₁-C₁₀ alkyl. In some embodiments, R₁₆ is substituted C₁-C₁₀ alkyl. In some embodiments, R₁₆ is unsubstituted C₂-C₁₀ alkynyl. In some embodiments, R₁₆ is substituted C₂-C₁₀ alkynyl. In some embodiments, R₁₆ is unsubstituted C₂-C₁₀ alkenyl. In some embodiments, R₁₆ is substituted C₂-C₁₀ alkenyl. In some embodiments, R₁₆ is unsubstituted carbohydrate. In some embodiments, R₁₆ is substituted carbohydrate. In some embodiments, R₁₆ is unsubstituted C₁-C₁₀ aliphatic acyl. In some embodiments, R₁₆ is substituted C₁-C₁₀ aliphatic acyl. In some embodiments, R₁₆ is unsubstituted C₆-C₁₀ aromatic acyl. In some embodiments, R₁₆ is substituted C₆-C₁₀ aromatic acyl. In some embodiments, R₁₆ is unsubstituted C₆-C₁₀ aralkyl acyl. In some embodiments, R₁₆ is substituted C₆-C₁₀ aralkyl acyl. In some embodiments, R₁₆ is unsubstituted C₆-C₁₀ alkylaryl acyl. In some embodiments, R₁₆ is substituted C₆-C₁₀ alkylaryl acyl. In some embodiments, R₁₆ is unsubstituted aryl. In some embodiments, R₁₆ is substituted aryl. In some embodiments, R₁₆ is unsubstituted C₃-C₁₀heterocyclyl. In some embodiments, R₁₆ is substituted C₃-C₁₀ heterocyclyl. In some embodiments, R₁₆ is unsubstituted heteroaryl. In some embodiments, R₁₆ is substituted heteroaryl. In some embodiments, R₁₆ is unsubstituted C₃-C₁₀cycloalkyl. In some embodiments, R₁₆ is substituted C₃-C₁₀cycloalkyl. In some embodiments, R₁₆ is —PO₃WY. In some embodiments, R₁₆ is —CH₂PO₄WY. In some embodiments, R₁₆ is —CH₂PO₄Z. In some embodiments, R₁₆ is —PO₃Z.

In some embodiments, R₁₈ is hydrogen. In some embodiments, R₁₈ is hydroxy. In some embodiments, R₁₈ is carboxaldehyde. In some embodiments, R₁₈ is unsubstituted amine. In some embodiments, R₁₈ is substituted amine. In some embodiments, R₁₈ is unsubstituted C₁-C₁₀ alkyl. In some embodiments, R₁₈ is unsubstituted C₂-C₁₀ alkynyl. In some embodiments, R₁₈ is substituted C₂-C₁₀ alkynyl. In some embodiments, R₁₈ is unsubstituted C₂-C₁₀ alkenyl. In some embodiments, R₁₈ is substituted C₂-C₁₀ alkenyl. In some embodiments, R₁₈ is carboxyl. In some embodiments, R₁₈ is unsubstituted carbohydrate. In some embodiments, R₁₈ is substituted carbohydrate. In some embodiments, R₁₈ is substituted carbohydrate. In some embodiments, R₁₈ is unsubstituted ester. In some embodiments, R₁₈ is substituted ester. In some embodiments, R₁₈ is unsubstituted acyloxy. In some embodiments, R₁₈ is substituted acyloxy. In some embodiments, R₁₈ is nitro. In some embodiments, R₁₈ is halogen. In some embodiments, R₁₈ is unsubstituted C₁-C₁₀ aliphatic acyl. In some embodiments, R₁₈ is substituted C₁-C₁₀ aliphatic acyl. In some embodiments, R₁₈ is unsubstituted C₆-C₁₀ aromatic acyl. In some embodiments, R₁₈ is substituted C₆-C₁₀ aromatic acyl. In some embodiments, R₁₈ is unsubstituted C₆-C₁₀ aralkyl acyl. In some embodiments, R₁₈ is substituted C₆-C₁₀ aralkyl acyl. In some embodiments, R₁₈ is unsubstituted C₆-C₁₀ alkylaryl acyl. In some embodiments, R₁₈ is substituted C₆-C₁₀ alkylaryl acyl. In some embodiments, R₁₈ is unsubstituted alkoxy. In some embodiments, R₁₈ is substituted alkoxy. In some embodiments, R₁₈ is unsubstituted aryl. In some embodiments, R₁₈ is substituted aryl. In some embodiments, R₁₈ is unsubstituted C₃-C₁₀heterocyclyl. In some embodiments, R₁₈ is substituted C₃-C₁₀heterocyclyl. In some embodiments, R₁₈ is unsubstituted heteroaryl. In some embodiments, R₁₈ is substituted heteroaryl. In some embodiments, R₁₈ is unsubstituted C₃-C₁₀cycloalkyl. In some embodiments, R₁ is substituted C₃-C₁₀cycloalkyl. In some embodiments, R₁₈ is —OPO₃WY. In some embodiments, R₁₈ is —OCH₂PO₄WY. In some embodiments, R₁₈ is —OCH₂PO₄Z. In some embodiments, R₁₈ is —OPO₃Z.

In some embodiments, R₁₉ is hydrogen. In some embodiments, R₁₉ is unsubstituted C₁-C₁₀ alkyl. In some embodiments, R₁₉ is substituted C₁-C₁₀ alkyl. In some embodiments, R₁₉ is unsubstituted C₂-C₁₀ alkynyl. In some embodiments, R₁₉ is substituted C₂-C₁₀ alkynyl. In some embodiments, R₁₉ is unsubstituted C₂-C₁₀ alkenyl. In some embodiments, R₁₉ is substituted C₂-C₁₀ alkenyl. In some embodiments, R₁₉ is unsubstituted carbohydrate. In some embodiments, R₁₉ is substituted carbohydrate. In some embodiments, R₁₉ is unsubstituted C₁-C₁₀ aliphatic acyl. In some embodiments, R₁₉ is substituted C₁-C₁₀ aliphatic acyl. In some embodiments, R₁₉ is unsubstituted C₆-C₁₀ aromatic acyl. In some embodiments, R₁₉ is substituted C₆-C₁₀ aromatic acyl. In some embodiments, R₁₉ is unsubstituted C₆-C₁₀ aralkyl acyl. In some embodiments, R₁₉ is substituted C₆-C₁₀ aralkyl acyl. In some embodiments, R₁₉ is unsubstituted C₆-C₁₀ alkylaryl acyl. In some embodiments, R₁₉ is substituted C₆-C₁₀ alkylaryl acyl. In some embodiments, R₁₉ is unsubstituted aryl. In some embodiments, R₁₉ is substituted aryl. In some embodiments, R₁₉ is unsubstituted C₃-C₁₀heterocyclyl. In some embodiments, R₁₉ is substituted C₃-C₁₀ heterocyclyl. In some embodiments, R₁₉ is unsubstituted heteroaryl. In some embodiments, R₁₉ is substituted heteroaryl. In some embodiments, R₁₉ is unsubstituted C₃-C₁₀cycloalkyl. In some embodiments, R₁₉ is substituted C₃-C₁₀cycloalkyl. In some embodiments, R₁₉ is —PO₃WY. In some embodiments, R₁₉ is —CH₂PO₄WY. In some embodiments, R₁₉ is —CH₂PO₄Z. In some embodiments, R₁₉ is —PO₃Z.

In some embodiments, s is an integer of 0. In some embodiments, s is an integer of 1. In some embodiments, s is an integer of 2. In some embodiments, s is an integer of 3.

In various embodiments, W is hydrogen. In various embodiments, W is unsubstituted methyl. In various embodiments, W is substituted methyl. In various embodiments, W is unsubstituted ethyl. In various embodiments, W is substituted ethyl. In various embodiments, W is unsubstituted alkyl. In various embodiments, W is substituted alkyl. In various embodiments, W is unsubstituted carbohydrate. In various embodiments, W is substituted carbohydrate. In various embodiments, W is potassium. In various embodiments, W is sodium. In various embodiments, W is lithium.

In various embodiments, Y is hydrogen. In various embodiments, Y is unsubstituted methyl. In various embodiments, Y is substituted methyl. In various embodiments, Y is unsubstituted ethyl. In various embodiments, Y is substituted ethyl. In various embodiments, Y is unsubstituted alkyl. In various embodiments, Y is substituted alkyl. In various embodiments, Y is unsubstituted carbohydrate. In various embodiments, Y is substituted carbohydrate. In various embodiments, Y is potassium. In various embodiments, Y is sodium. In various embodiments, Y is lithium.

In various embodiments, Z is calcium. In various embodiments, Z is magnesium. In various embodiments, Z is iron.

In some embodiments of the invention, the pyrone analog is of Formula IX or a pharmaceutically/veterinarily acceptable salt thereof:

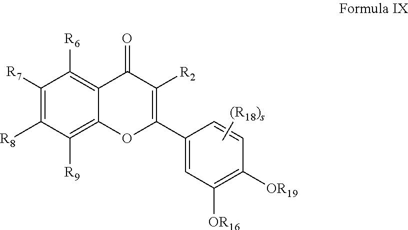

wherein R₂ is hydrogen, hydroxyl, C₁-C₁₀ alkyl, C₂-C₁₀ alkynyl, C₂-C₁₀ alkenyl, carboxyl, carbohydrate, ester, acyloxy, nitro, halogen, C₁-C₁₀ aliphatic acyl, C₆-C₁₀ aromatic acyl, C₆-C₁₀ aralkyl acyl, C₆-C₁₀ alkylaryl acyl, alkoxy, amine, aryl, C₄-C₁₀heterocyclyl, heteroaryl, C₃-C₁₀cycloalkyl, —OPO₃WY, —OCH₂PO₄WY, —OCH₂PO₄Z or —OPO₃Z;

R₆, R₇, R₈, and R₉ are independently hydrogen, carboxaldehyde, amino, C₁-C₁₀ alkyl, C₂-C₁₀ alkynyl, C₂-C₁₀ alkenyl, carboxyl, carbohydrate, ester, acyloxy, nitro, halogen, C₁-C₁₀ aliphatic acyl, C₆-C₁₀ aromatic acyl, C₆-C₁₀ aralkyl acyl, C₆-C₁₀ alkylaryl acyl, alkoxy, amine, aryl, C₃-C₁₀ heterocyclyl, heteroaryl, C₃-C₁₀cycloalkyl, —OPO₃WY, —OCH₂PO₄WY, —OCH₂PO₄Z or —OPO₃Z;

wherein R₁₆ is hydrogen, C₁-C₁₀ alkyl, C₂-C₁₀ alkynyl, C₂-C₁₀ alkenyl, carbohydrate, C₁-C₁₀ aliphatic acyl, C₆-C₁₀ aromatic acyl, C₆-C₁₀ aralkyl acyl, C₆-C₁₀ alkylaryl acyl, aryl, C₃-C₁₀ heterocyclyl, heteroaryl, C₃-C₁₀cycloalkyl, —PO₃WY, —CH₂PO₄WY, —CH₂PO₄Z or —PO₃Z;

each instance of R₁₈ is independently hydrogen, hydroxyl, carboxaldehyde, amine, C₁-C₁₀ alkyl, C₂-C₁₀ alkynyl, C₂-C₁₀ alkenyl, carboxyl, carbohydrate, ester, acyloxy, nitro, halogen, C₁-C₁₀ aliphatic acyl, C₆-C₁₀ aromatic acyl, C₆-C₁₀ aralkyl acyl, C₆-C₁₀alkylaryl acyl, alkoxy, alkyl, phosphate, aryl, heteroaryl, C₃-C₁₀ heterocyclic, C₃-C₁₀cycloalkyl, —OPO₃WY, —OCH₂PO₄WY, —OCH₂PO₄Z or —OPO₃Z;

R₁₉ is hydrogen, C₁-C₁₀ alkyl, C₂-C₁₀ alkynyl, C₂-C₁₀ alkenyl, carbohydrate, C₁-C₁₀ aliphatic acyl, C₆-C₁₀ aromatic acyl, C₆-C₁₀ aralkyl acyl, C₆-C₁₀ alkylaryl acyl, aryl, C₃-C₁₀heterocyclyl, heteroaryl, optionally substituted C₃-C₁₀cycloalkyl, —PO₃WY, —CH₂PO₄WY, —CH₂PO₄Z or —PO₃Z;

s is an integer of 0, 1, 2, or 3;

W and Y are independently hydrogen, methyl, ethyl, alkyl, carbohydrate, or a cation, and Z is a multivalent cation; and

wherein the compound of Formula IX is not the compound of Formula A:

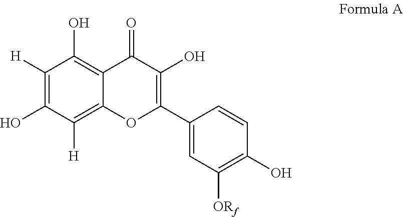

wherein R_{f} is hydrogen; and

wherein the compound of Formula IX is not the compound wherein:

R₂ is hydrogen, R₆, R₇, R₈, and R₉ are hydrogen, R₁₆ is hydrogen, R₁₇ is hydroxy, and s is 0;

R₂ is hydrogen, R₆, R₇, R₈, and R₉ are hydrogen, R₁₆ is hydrogen, R₁₇ is hydroxy, s is 1, and R₁₈ is 5′-hydroxy;

R₂ is hydrogen, R₆, R₇, R₈, and R₉ are hydrogen, R₁₆ is hydrogen, R₁₇ is hydroxy, s is 1, and R₁₈ is 5′-nitro;

R₂ is hydroxy, R₆, R₇, R₈, and R₉ are hydrogen, R₁₆ is hydrogen, R₁₇ is hydroxy, and s is 0;

R₂ is hydroxy, R₆, R₇, R₈, and R₉ are hydrogen, R₁₆ is hydrogen, R₁₇ is hydroxy, s is 1, and R₁₈ is 5′-hydroxy;

R₂ is hydrogen, R₆ and R₈ are hydroxy, R₇ and R₉ are hydrogen, R₁₆ is hydrogen, R₁₇ is hydroxy, and s is 0;

R₂ is hydrogen, R₆ and R₈ are hydroxy, R₇ and R₉ are hydrogen, R₁₆ is hydrogen, R₁₇ is hydroxy, s is 1, and

R₁₈ is 5′-hydroxy;

R₂ is hydrogen, R₆ and R₈ are hydroxy, R₇ and R₉ are hydrogen, R₁₆ is hydrogen, R₁₇ is hydroxy, s is 1, and

R₁₈ is 5′-methoxy;

R₂ is hydroxy, R₆ and R₈ are hydroxy, R₇ and R₉ are hydrogen, R₁₆ is methyl, R₁₇ is hydroxy, and s is 0;

R₂ is hydroxy, R₆ and R₈ are hydroxy, R₇ and R₉ are hydrogen, R₁₆ is hydrogen, R₁₇ is methoxy, and s is 0;

R₂ is hydrogen, R₆, R₈, and R₉ are hydrogen, R₇ is hydroxy, R₁₆ is hydrogen, R₁₇ is hydroxy, and s is 0;

R₂ is hydrogen, R₆, R₈, and R₉ are hydrogen, R₇ is hydroxy, R₁₆ is hydrogen, R₁₇ is hydroxy, s is 1, and R₁₈ is 5′-hydroxy;

R₂ is hydroxy, R₆, R₈, and R₉ are hydrogen, R₇ is hydroxy, R₁₆ is hydrogen, R₁₇ is hydroxy, and s is 0;

R₂ is hydrogen, R₆, R₇, and R₉ are hydrogen, R₈ is hydroxy, R₁₆ is hydrogen, R₁₇ is hydroxy, and s is 0;

R₂ is hydrogen, R₆, R₇, and R₉ are hydrogen, R₈ is hydroxy, R₁₆ is hydrogen, R₁₇ is hydroxy, s is 1, and R₁₈ is 5′-hydroxy;

R₂ is hydroxy, R₆, R₇, and R₉ are hydrogen, R₈ is hydroxy, R₁₆ is hydrogen, R₁₇ is hydroxy, and s is 0; or

R₂ is hydroxy, R₆, R₇, and R₉ are hydrogen, R₈ is hydroxy, R₁₆ is hydrogen, R₁₇ is hydroxy, s is 1, and R₁₈ is 5′-hydroxy.

In some embodiments, the compound of Formula VIII is not the compound of Formula A wherein R_{f} is PO₃K₂.

In some embodiments of the invention, at least one of R₁₆ and R₁₉ is —PO₃WY, —CH₂PO₄WY, —CH₂PO₄Z or —PO₃Z, or at least one of R₁₇ or R₁₈ is —OPO₃WY, —OCH₂PO₄WY, —OCH₂PO₄Z or —OPO₃Z. In some embodiments, at least one of R₁₆ and R₁₉ is —PO₃WY, —CH₂PO₄WY, —CH₂PO₄Z or —PO₃Z and at least one of R₁₇ or R₁₈ is —OPO₃WY, —OCH₂PO₄WY, —OCH₂PO₄Z or —OPO₃Z.

In some embodiments, R₂ is hydrogen. In some embodiments, R₂ is hydroxyl. In some embodiments, R₂ is optionally substituted C₁-C₁₀ alkyl. In some embodiments, R₂ is unsubstituted C₁-C₁₀ alkyl. In some embodiments, R₂ is substituted C₁-C₁₀ alkyl. In some embodiments, R₂ is unsubstituted C₁-C₁₀ alkyl. In some other embodiments, R₂ is substituted C₁-C₁₀ alkyl. In some embodiments, R₂ is unsubstituted C₂-C₁₀ alkynyl. In some embodiments, R₂ is substituted C₂-C₁₀ alkynyl. In some embodiments, R₂ is unsubstituted C₂-C₁₀ alkenyl. In some embodiments, R₂ is substituted C₂-C₁₀ alkenyl. In some embodiments, R₂ is carboxyl. In some embodiments, R₂ is unsubstituted carbohydrate. In some embodiments, R₂ is substituted carbohydrate. In some embodiments, R₂ is unsubstituted ester. In some embodiments, R₂ is substituted ester. In some embodiments, R₂ is unsubstituted acyloxy. In some embodiments, R₂ is substituted acyloxy. In some embodiments, R₂ is nitro. In some embodiments, R₂ is halogen. In some embodiments, R₂ is unsubstituted C₁-C₁₀ aliphatic acyl. In some embodiments, R₂ is substituted C₁-C₁₀ aliphatic acyl. In some embodiments, R₂ is unsubstituted C₆-C₁₀ aromatic acyl. In some embodiments, R₂ is substituted C₆-C₁₀ aromatic acyl. In some embodiments, R₂ is unsubstituted C₆-C₁₀ aralkyl acyl. In some embodiments, R₂ is substituted C₆-C₁₀ aralkyl acyl. In some embodiments, R₂ is unsubstituted C₆-C₁₀ alkylaryl acyl. In some embodiments, R₂ is substituted C₆-C₁₀ alkylaryl acyl. In some embodiments, R₂ is unsubstituted alkoxy. In some embodiments, R₂ is substituted alkoxy. In some embodiments, R₂ is unsubstituted amine. In some embodiments, R₂ is substituted amine. In some embodiments, R₂ is unsubstituted aryl. In some embodiments, R₂ is substituted aryl. In some embodiments, R₂ is unsubstituted C₄-C₁₀heterocyclyl. In some embodiments, R₂ is substituted C₄-C₁₀heterocyclyl. In some embodiments, R₂ is unsubstituted heteroaryl. In some embodiments, R₂ is substituted heteroaryl. In some embodiments, R₂ is unsubstituted C₃-C₁₀cycloalkyl. In some embodiments, R₂ is substituted C₃-C₁₀cycloalkyl. In some embodiments, R₂ is —OPO₃WY. In some embodiments, R₂ is —OCH₂PO₄WY. In some embodiments, R₂ is —OCH₂PO₄Z. In some embodiments, R₂ is —OPO₃Z.

In some embodiments, R₆ is hydrogen. In some embodiments, R₆ is carboxaldehyde. In some embodiments, R₆ is unsubstituted amine. In some embodiments, R₆ is substituted amine. In some embodiments, R₆ is unsubstituted C₁-C₁₀ alkyl. In some embodiments, R₆ is substituted C₁-C₁₀ alkyl. In some embodiments, R₆ is unsubstituted C₂-C₁₀ alkynyl. In some embodiments, R₆ is substituted C₂-C₁₀ alkynyl. In some embodiments, R₆ is unsubstituted C₂-C₁₀ alkenyl. In some embodiments, R₆ is substituted C₂-C₁₀ alkenyl. In some embodiments, R₆ is carboxyl. In some embodiments, R₆ is unsubstituted carbohydrate. In some embodiments, R₆ is substituted carbohydrate. In some embodiments, R₆ is unsubstituted ester. In some embodiments, R₆ is substituted ester. In some embodiments, R₆ is unsubstituted acyloxy. In some embodiments, R₆ is substituted acyloxy. In some embodiments, R₆ is nitro. In some embodiments, R₆ is halogen. In some embodiments, R₆ is unsubstituted C₁-C₁₀ aliphatic acyl. In some embodiments, R₆ is substituted C₁-C₁₀ aliphatic acyl. In some embodiments, R₆ is unsubstituted C₆-C₁₀ aromatic acyl. In some embodiments, R₆ is substituted C₆-C₁₀ aromatic acyl. In some embodiments, R₆ is unsubstituted C₆-C₁₀ aralkyl acyl. In some embodiments, R₆ is substituted C₆-C₁₀ aralkyl acyl. In some embodiments, R₆ is unsubstituted C₆-C₁₀ alkylaryl acyl. In some embodiments, R₆ is substituted C₆-C₁₀ alkylaryl acyl. In some embodiments, R₆ is unsubstituted alkoxy. In some embodiments, R₆ is substituted alkoxy. In some embodiments, R₆ is unsubstituted aryl. In some embodiments, R₆ is substituted aryl. In some embodiments, R₆ is unsubstituted C₃-C₁₀heterocyclyl. In some embodiments, R₆ is substituted C₃-C₁₀heterocyclyl. In some embodiments, R₆ is unsubstituted heteroaryl. In some embodiments, R₆ is unsubstituted C₃-C₁₀cycloalkyl. In some embodiments, R₆ is substituted C₃-C₁₀cycloalkyl. In some embodiments, R₆ is —OPO₃WY. In some embodiments, R₆ is —OCH₂PO₄WY. In some embodiments, R₆ is —OCH₂PO₄Z. In some embodiments, R₆ is —OPO₃Z.

In some embodiments, R₇ is hydrogen. In some embodiments, R₇ is carboxaldehyde. In some embodiments, R₇ is unsubstituted amine. In some embodiments, R₇ is substituted amine. In some embodiments, R₇ is unsubstituted C₁-C₁₀ alkyl. In some embodiments, R₇ is substituted C₁-C₁₀ alkyl. In some embodiments, R₇ is unsubstituted C₂-C₁₀ alkynyl. In some embodiments, R₇ is substituted C₂-C₁₀ alkynyl. In some embodiments, R₇ is unsubstituted C₂-C₁₀ alkenyl. In some embodiments, R₇ is substituted C₂-C₁₀ alkenyl. In some embodiments, R₇ is carboxyl. In some embodiments, R₇ is unsubstituted carbohydrate. In some embodiments, R₇ is substituted carbohydrate. In some embodiments, R₇ is unsubstituted ester. In some embodiments, R₇ is substituted ester. In some embodiments, R₇ is unsubstituted acyloxy. In some embodiments, R₇ is substituted acyloxy. In some embodiments, R₇ is nitro. In some embodiments, R₇ is halogen. In some embodiments, R₇ is unsubstituted C₁-C₁₀ aliphatic acyl. In some embodiments, R₇ is substituted C₁-C₁₀ aliphatic acyl. In some embodiments, R₇ is unsubstituted C₆-C₁₀ aromatic acyl. In some embodiments, R₇ is substituted C₆-C₁₀ aromatic acyl. In some embodiments, R₇ is unsubstituted C₆-C₁₀ aralkyl acyl. In some embodiments, R₇ is substituted C₆-C₁₀ aralkyl acyl. In some embodiments, R₇ is unsubstituted C₆-C₁₀ alkylaryl acyl. In some embodiments, R₇ is substituted C₆-C₁₀ alkylaryl acyl. In some embodiments, R₇ is unsubstituted alkoxy. In some embodiments, R₇ is substituted alkoxy. In some embodiments, R₇ is unsubstituted aryl. In some embodiments, R₇ is substituted aryl. In some embodiments, R₇ is unsubstituted C₃-C₁₀heterocyclyl. In some embodiments, R₇ is substituted C₃-C₁₀heterocyclyl. In some embodiments, R₇ is unsubstituted heteroaryl, In some embodiments, R₇ is unsubstituted C₃-C₁₀cycloalkyl. In some embodiments, R₇ is substituted C₃-C₁₀cycloalkyl. In some embodiments, R₇ is —OPO₃WY. In some embodiments, R₇ is —OCH₂PO₄WY. In some embodiments, R₇ is —OCH₂PO₄Z. In some embodiments, R₇ is —OPO₃Z.

In some embodiments, R₈ is hydrogen. In some embodiments, R₈ is carboxaldehyde. In some embodiments, R₈ is unsubstituted amine. In some embodiments, R₈ is substituted amine. In some embodiments, R₅ is unsubstituted C₁-C₁₀ alkyl. In some embodiments, R₈ is substituted C₁-C₁₀ alkyl. In some embodiments, R₈ is unsubstituted C₂-C₁₀ alkynyl. In some embodiments, R₈ is substituted C₂-C₁₀ alkynyl. In some embodiments, R₈ is unsubstituted C₂-C₁₀ alkenyl. In some embodiments, R₈ is substituted C₂-C₁₀ alkenyl. In some embodiments, R₈ is carboxyl. In some embodiments, R₈ is unsubstituted carbohydrate. In some embodiments, R₈ is substituted carbohydrate. In some embodiments, R₈ is unsubstituted ester. In some embodiments, R₈ is substituted ester. In some embodiments, R₈ is unsubstituted acyloxy. In some embodiments, R₈ is substituted acyloxy. In some embodiments, R₈ is nitro. In some embodiments, R₈ is halogen. In some embodiments, R₈ is unsubstituted C₁-C₁₀ aliphatic acyl. In some embodiments, R₈ is substituted C₁-C₁₀ aliphatic acyl. In some embodiments, R₈ is unsubstituted C₆-C₁₀ aromatic acyl. In some embodiments, R₈ is substituted C₆-C₁₀ aromatic acyl. In some embodiments, R₈ is unsubstituted C₆-C₁₀ aralkyl acyl. In some embodiments, R₈ is substituted C₆-C₁₀ aralkyl acyl. In some embodiments, R₈ is unsubstituted C₆-C₁₀ alkylaryl acyl. In some embodiments, R₈ is substituted C₆-C₁₀ alkylaryl acyl. In some embodiments, R₈ is unsubstituted alkoxy. In some embodiments, R₈ is substituted alkoxy. In some embodiments, R₈ is unsubstituted aryl. In some embodiments, R₈ is substituted aryl. In some embodiments, R₈ is unsubstituted C₃-C₁₀heterocyclyl. In some embodiments, R₈ is substituted C₃-C₁₀heterocyclyl. In some embodiments, R₈ is unsubstituted heteroaryl, In some embodiments, R₈ is unsubstituted C₃-C₁₀cycloalkyl. In some embodiments, R₈ is substituted C₃-C₁₀cycloalkyl. In some embodiments, R₈ is —OPO₃WY. In some embodiments, R₈ is —OCH₂PO₄WY. In some embodiments, R₈ is —OCH₂PO₄Z. In some embodiments, R₈ is —OPO₃Z.

In some embodiments, R₉ is hydrogen. In some embodiments, R₉ is carboxaldehyde. In some embodiments, R₉ is unsubstituted amine. In some embodiments, R₉ is substituted amine. In some embodiments, R₉ is unsubstituted C₁-C₁₀ alkyl. In some embodiments, R₉ is substituted C₁-C₁₀ alkyl. In some embodiments, R₉ is unsubstituted C₂-C₁₀ alkynyl. In some embodiments, R₉ is substituted C₂-C₁₀ alkynyl. In some embodiments, R₉ is unsubstituted C₂-C₁₀ alkenyl. In some embodiments, R₉ is substituted C₂-C₁₀ alkenyl. In some embodiments, R₉ is carboxyl. In some embodiments, R₉ is unsubstituted carbohydrate. In some embodiments, R₉ is substituted carbohydrate. In some embodiments, R₉ is unsubstituted ester. In some embodiments, R₉ is substituted ester. In some embodiments, R₉ is unsubstituted acyloxy. In some embodiments, R₉ is substituted acyloxy. In some embodiments, R₉ is nitro. In some embodiments, R₉ is halogen. In some embodiments, R₉ is unsubstituted C₁-C₁₀ aliphatic acyl. In some embodiments, R₉ is substituted C₁-C₁₀ aliphatic acyl. In some embodiments, R₉ is unsubstituted C₆-C₁₀ aromatic acyl. In some embodiments, R₉ is substituted C₆-C₁₀ aromatic acyl. In some embodiments, R₉ is unsubstituted C₆-C₁₀ aralkyl acyl. In some embodiments, R₉ is substituted C₆-C₁₀ aralkyl acyl. In some embodiments, R₉ is unsubstituted C₆-C₁₀ alkylaryl acyl. In some embodiments, R₉ is substituted C₆-C₁₀ alkylaryl acyl. In some embodiments, R₉ is unsubstituted alkoxy. In some embodiments, R₉ is substituted alkoxy. In some embodiments, R₉ is unsubstituted aryl. In some embodiments, R₉ is substituted aryl. In some embodiments, R₉ is unsubstituted C₃-C₁₀heterocyclyl. In some embodiments, R₉ is substituted C₃-C₁₀heterocyclyl. In some embodiments, R₉ is unsubstituted heteroaryl, In some embodiments, R₉ is unsubstituted C₃-C₁₀cycloalkyl. In some embodiments, R₉ is substituted C₃-C₁₀cycloalkyl. In some embodiments, R₉ is —OPO₃WY. In some embodiments, R₉ is —OCH₂PO₄WY. In some embodiments, R₉ is —OCH₂PO₄Z. In some embodiments, R₉ is —OPO₃Z.

In some embodiments, R₁₆ is hydrogen. In some embodiments, R₁₆ is unsubstituted C₁-C₁₀ alkyl. In some embodiments, R₁₆ is substituted C₁-C₁₀ alkyl. In some embodiments, R₁₆ is unsubstituted C₂-C₁₀ alkynyl. In some embodiments, R₁₆ is substituted C₂-C₁₀ alkynyl. In some embodiments, R₁₆ is unsubstituted C₂-C₁₀ alkenyl. In some embodiments, R₁₆ is substituted C₂-C₁₀ alkenyl. In some embodiments, R₁₆ is unsubstituted carbohydrate. In some embodiments, R₁₆ is substituted carbohydrate. In some embodiments, R₁₆ is unsubstituted C₁-C₁₀ aliphatic acyl. In some embodiments, R₁₆ is substituted C₁-C₁₀ aliphatic acyl. In some embodiments, R₁₆ is unsubstituted C₆-C₁₀ aromatic acyl. In some embodiments, R₁₆ is substituted C₆-C₁₀ aromatic acyl. In some embodiments, R₁₆ is unsubstituted C₆-C₁₀ aralkyl acyl. In some embodiments, R₁₆ is substituted C₆-C₁₀ aralkyl acyl. In some embodiments, R₁₆ is unsubstituted C₆-C₁₀ alkylaryl acyl. In some embodiments, R₁₆ is substituted C₆-C₁₀ alkylaryl acyl. In some embodiments, R₁₆ is unsubstituted aryl. In some embodiments, R₁₆ is substituted aryl. In some embodiments, R₁₆ is unsubstituted C₃-C₁₀heterocyclyl. In some embodiments, R₁₆ is substituted C₃-C₁₀ heterocyclyl. In some embodiments, R₁₆ is unsubstituted heteroaryl. In some embodiments, R₁₆ is substituted heteroaryl. In some embodiments, R₁₆ is unsubstituted C₃-C₁₀cycloalkyl. In some embodiments, R₁₆ is substituted C₃-C₁₀cycloalkyl. In some embodiments, R₁₆ is —PO₃WY. In some embodiments, R₁₆ is —CH₂PO₄WY. In some embodiments, R₁₆ is —CH₂PO₄Z. In some embodiments, R₁₆ is —PO₃Z.

In some embodiments, R₁₈ is hydrogen. In some embodiments, R₁₈ is hydroxy. In some embodiments, R₁₈ is carboxaldehyde. In some embodiments, R₁₈ is unsubstituted amine. In some embodiments, R₁₈ is substituted amine. In some embodiments, R₁₈ is unsubstituted C₁-C₁₀ alkyl. In some embodiments, R₁₈ is unsubstituted C₂-C₁₀ alkynyl. In some embodiments, R₁₈ is substituted C₂-C₁₀ alkynyl. In some embodiments, R₁₈ is unsubstituted C₂-C₁₀ alkenyl. In some embodiments, R₁₈ is substituted C₂-C₁₀ alkenyl. In some embodiments, R₁₈ is carboxyl. In some embodiments, R₁₈ is unsubstituted carbohydrate. In some embodiments, R₁₈ is substituted carbohydrate. In some embodiments, R₁₈ is substituted carbohydrate. In some embodiments, R₁₈ is unsubstituted ester. In some embodiments, R₁₈ is substituted ester. In some embodiments, R₁₈ is unsubstituted acyloxy. In some embodiments, R₁₈ is substituted acyloxy. In some embodiments, R₁₈ is nitro. In some embodiments, R₁₈ is halogen. In some embodiments, R₁₈ is unsubstituted C₁-C₁₀ aliphatic acyl. In some embodiments, R₁₈ is substituted C₁-C₁₀ aliphatic acyl. In some embodiments, R₁₈ is unsubstituted C₆-C₁₀ aromatic acyl. In some embodiments, R₁₈ is substituted C₆-C₁₀ aromatic acyl. In some embodiments, R₁₈ is unsubstituted C₆-C₁₀ aralkyl acyl. In some embodiments, R₁₈ is substituted C₆-C₁₀ aralkyl acyl. In some embodiments, R₁₈ is unsubstituted C₆-C₁₀ alkylaryl acyl. In some embodiments, R₁₈ is substituted C₆-C₁₀ alkylaryl acyl. In some embodiments, R₁₈ is unsubstituted alkoxy. In some embodiments, R₁₈ is substituted alkoxy. In some embodiments, R₁₈ is unsubstituted aryl. In some embodiments, R₁₈ is substituted aryl. In some embodiments, R₁₈ is unsubstituted C₃-C₁₀heterocyclyl. In some embodiments, R₁₈ is substituted C₃-C₁₀heterocyclyl. In some embodiments, R₁₈ is unsubstituted heteroaryl. In some embodiments, R₁₈ is substituted heteroaryl. In some embodiments, R₁₈ is unsubstituted C₃-C₁₀cycloalkyl. In some embodiments, R₁₈ is substituted C₃-C₁₀cycloalkyl. In some embodiments, R₁₈ is —OPO₃WY. In some embodiments, R₁₈ is —OCH₂PO₄WY. In some embodiments, R₁₈ is —OCH₂PO₄Z. In some embodiments, R₁₈ is —OPO₃Z.

In some embodiments, R₁₉ is hydrogen. In some embodiments, R₁₉ is unsubstituted C₁-C₁₀ alkyl. In some embodiments, R₁₉ is substituted C₁-C₁₀ alkyl. In some embodiments, R₁₉ is unsubstituted C₂-C₁₀ alkynyl. In some embodiments, R₁₉ is substituted C₂-C₁₀ alkynyl. In some embodiments, R₁₉ is unsubstituted C₂-C₁₀ alkenyl. In some embodiments, R₁₉ is substituted C₂-C₁₀ alkenyl. In some embodiments, R₁₉ is unsubstituted carbohydrate. In some embodiments, R₁₉ is substituted carbohydrate. In some embodiments, R₁₉ is unsubstituted C₁-C₁₀ aliphatic acyl. In some embodiments, R₁₉ is substituted C₁-C₁₀ aliphatic acyl. In some embodiments, R₁₉ is unsubstituted C₆-C₁₀ aromatic acyl. In some embodiments, R₁₉ is substituted C₆-C₁₀ aromatic acyl. In some embodiments, R₁₉ is unsubstituted C₆-C₁₀ aralkyl acyl. In some embodiments, R₁₉ is substituted C₆-C₁₀ aralkyl acyl. In some embodiments, R₁₉ is unsubstituted C₆-C₁₀ alkylaryl acyl. In some embodiments, R₁₉ is substituted C₆-C₁₀ alkylaryl acyl. In some embodiments, R₁₉ is unsubstituted aryl. In some embodiments, R₁₉ is substituted aryl. In some embodiments, R₁₉ is unsubstituted C₃-C₁₀heterocyclyl. In some embodiments, R₁₉ is substituted C₃-C₁₀ heterocyclyl. In some embodiments, R₁₉ is unsubstituted heteroaryl. In some embodiments, R₁₉ is substituted heteroaryl. In some embodiments, R₁₉ is unsubstituted C₃-C₁₀cycloalkyl. In some embodiments, R₁₉ is substituted C₃-C₁₀cycloalkyl. In some embodiments, R₁₉ is —PO₃WY. In some embodiments, R₁₉ is —CH₂PO₄WY. In some embodiments, R₁₉ is —CH₂PO₄Z. In some embodiments, R₁₉ is —PO₃Z.

In some embodiments, s is an integer of 0. In some embodiments, s is an integer of 1. In some embodiments, s is an integer of 2. In some embodiments, s is an integer of 3.

In various embodiments, W is hydrogen. In various embodiments, W is unsubstituted methyl. In various embodiments, W is substituted methyl. In various embodiments, W is unsubstituted ethyl. In various embodiments, W is substituted ethyl. In various embodiments, W is unsubstituted alkyl. In various embodiments, W is substituted alkyl. In various embodiments, W is unsubstituted carbohydrate. In various embodiments, W is substituted carbohydrate. In various embodiments, W is potassium. In various embodiments, W is sodium. In various embodiments, W is lithium.

In various embodiments, Y is hydrogen. In various embodiments, Y is unsubstituted methyl. In various embodiments, Y is substituted methyl. In various embodiments, Y is unsubstituted ethyl. In various embodiments, Y is substituted ethyl. In various embodiments, Y is unsubstituted alkyl. In various embodiments, Y is substituted alkyl. In various embodiments, Y is unsubstituted carbohydrate. In various embodiments, Y is substituted carbohydrate. In various embodiments, Y is potassium. In various embodiments, Y is sodium. In various embodiments, Y is lithium.

In various embodiments, Z is calcium. In various embodiments, Z is magnesium. In various embodiments, Z is iron.

In some embodiments of the invention, the pyrone analog is of Formula X or a pharmaceutically/veterinarily acceptable salt thereof:

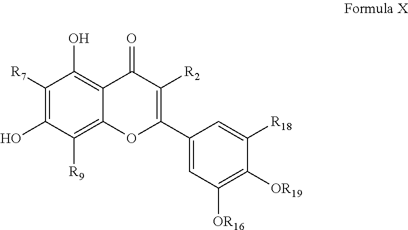

wherein R₂ is hydrogen, hydroxyl, C₁-C₁₀ alkyl, C₂-C₁₀ alkynyl, C₂-C₁₀ alkenyl, carboxyl, carbohydrate, ester, acyloxy, nitro, halogen, C₁-C₁₀ aliphatic acyl, C₆-C₁₀ aromatic acyl, C₆-C₁₀ aralkyl acyl, C₆-C₁₀ alkylaryl acyl, alkoxy, amine, aryl, C₄-C₁₀heterocyclyl, heteroaryl, C₃-C₁₀cycloalkyl, —OPO₃WY, —OCH₂PO₄WY, —OCH₂PO₄Z or —OPO₃Z;

R₇ and R₉ are independently hydrogen, hydroxyl, carboxaldehyde, amino, C₁-C₁₀ alkyl, C₂-C₁₀ alkynyl, C₂-C₁₀ alkenyl, carboxyl, carbohydrate, ester, acyloxy, nitro, halogen, C₁-C₁₀ aliphatic acyl, C₆-C₁₀ aromatic acyl, C₆-C₁₀ aralkyl acyl, C₆-C₁₀ alkylaryl acyl, alkoxy, amine, aryl, C₃-C₁₀ heterocyclyl, heteroaryl, C₃-C₁₀cycloalkyl, —OPO₃WY, —OCH₂PO₄WY, —OCH₂PO₄Z or —OPO₃Z;

wherein R₁₆ is hydrogen, C₁-C₁₀ alkyl, C₂-C₁₀ alkynyl, C₂-C₁₀ alkenyl, carbohydrate, C₁-C₁₀ aliphatic acyl, C₆-C₁₀ aromatic acyl, C₆-C₁₀ aralkyl acyl, C₆-C₁₀ alkylaryl acyl, aryl, C₃-C₁₀ heterocyclyl, heteroaryl, C₃-C₁₀cycloalkyl, —PO₃WY, —CH₂PO₄WY, —CH₂PO₄Z or —PO₃Z;

each instance of R₁₈ is independently hydrogen, hydroxyl, carboxaldehyde, amine, C₁-C₁₀ alkyl, C₂-C₁₀ alkynyl, C₂-C₁₀ alkenyl, carboxyl, carbohydrate, ester, acyloxy, nitro, halogen, C₁-C₁₀ aliphatic acyl, C₆-C₁₀ aromatic acyl, C₆-C₁₀ aralkyl acyl, C₆-C₁₀alkylaryl acyl, alkoxy, alkyl, phosphate, aryl, heteroaryl, C₃-C₁₀ heterocyclic, C₃-C₁₀cycloalkyl, —OPO₃WY, —OCH₂PO₄WY, —OCH₂PO₄Z or —OPO₃Z;

R₁₉ is hydrogen, C₁-C₁₀ alkyl, C₂-C₁₀ alkynyl, C₂-C₁₀ alkenyl, carbohydrate, C₁-C₁₀ aliphatic acyl, C₆-C₁₀ aromatic acyl, C₆-C₁₀ aralkyl acyl, C₆-C₁₀ alkylaryl acyl, aryl, C₃-C₁₀heterocyclyl, heteroaryl, optionally substituted C₃-C₁₀cycloalkyl, —PO₃WY, —CH₂PO₄WY, —CH₂PO₄Z or —PO₃Z;

s is an integer of 0, 1, 2, or 3; and

W and Y are independently hydrogen, methyl, ethyl, alkyl, carbohydrate, or a cation, and Z is a multivalent cation; and

wherein the compound of Formula X is not the compound of Formula A:

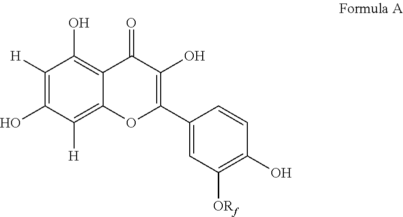

wherein R_{f} is hydrogen; and

wherein the compound of Formula X is not the compound wherein:

R₂ is hydrogen, R₇ and R₉ are hydrogen, R₁₆ is hydrogen, R₁₉ is hydrogen, and R₁₈ is hydrogen;

R₂ is hydrogen, R₇ and R₉ are hydrogen, R₁₆ is hydrogen, R₁₉ is hydrogen, and R₁₈ is hydroxy;

R₂ is hydrogen, R₇ and R₉ are hydrogen, R₁₆ is hydrogen, R₁₉ is hydrogen, and R₁₈ is methoxy;

R₂ is hydroxy, R₇ and R₉ are hydrogen, R₁₆ is methyl, R₁₉ is hydrogen, and R₁₈ is hydrogen; or

R₂ is hydroxy, R₇ and R₉ are hydrogen, R₁₆ is hydrogen, R₁₉ is methyl, and R₁₈ is hydrogen.

In some embodiments, the compound of Formula X is not the compound of Formula A wherein R_{f} is PO₃K₂.

In some embodiments of the invention, at least one of R₁₆ and R₁₉ is —PO₃WY, —CH₂PO₄WY, —CH₂PO₄Z or —PO₃Z, or at least one of R₁₇ or R₁₈ is —OPO₃WY, —OCH₂PO₄WY, —OCH₂PO₄Z or —OPO₃Z. In some embodiments, at least one of R₁₆ and R₁₉ is —PO₃WY, —CH₂PO₄WY, —CH₂PO₄Z or —PO₃Z and at least one of R₁₇ or R₁₈ is —OPO₃WY, —OCH₂PO₄WY, —OCH₂PO₄Z or —OPO₃Z.

In some embodiments, R₂ is hydrogen. In some embodiments, R₂ is hydroxyl. In some embodiments, R₂ is optionally substituted C₁-C₁₀ alkyl. In some embodiments, R₂ is unsubstituted C₁-C₁₀ alkyl. In some embodiments, R₂ is substituted C₁-C₁₀ alkyl. In some embodiments, R₂ is unsubstituted C₁-C₁₀ alkyl. In some other embodiments, R₂ is substituted C₁-C₁₀ alkyl. In some embodiments, R₂ is unsubstituted C₂-C₁₀ alkynyl. In some embodiments, R₂ is substituted C₂-C₁₀ alkynyl. In some embodiments, R₂ is unsubstituted C₂-C₁₀ alkenyl. In some embodiments, R₂ is substituted C₂-C₁₀ alkenyl. In some embodiments, R₂ is carboxyl. In some embodiments, R₂ is unsubstituted carbohydrate. In some embodiments, R₂ is substituted carbohydrate. In some embodiments, R₂ is unsubstituted ester. In some embodiments, R₂ is substituted ester. In some embodiments, R₂ is unsubstituted acyloxy. In some embodiments, R₂ is substituted acyloxy. In some embodiments, R₂ is nitro. In some embodiments, R₂ is halogen. In some embodiments, R₂ is unsubstituted C₁-C₁₀ aliphatic acyl. In some embodiments, R₂ is substituted C₁-C₁₀ aliphatic acyl. In some embodiments, R₂ is unsubstituted C₆-C₁₀ aromatic acyl. In some embodiments, R₂ is substituted C₆-C₁₀ aromatic acyl. In some embodiments, R₂ is unsubstituted C₆-C₁₀ aralkyl acyl. In some embodiments, R₂ is substituted C₆-C₁₀ aralkyl acyl. In some embodiments, R₂ is unsubstituted C₆-C₁₀ alkylaryl acyl. In some embodiments, R₂ is substituted C₆-C₁₀ alkylaryl acyl. In some embodiments, R₂ is unsubstituted alkoxy. In some embodiments, R₂ is substituted alkoxy. In some embodiments, R₂ is unsubstituted amine. In some embodiments, R₂ is substituted amine. In some embodiments, R₂ is unsubstituted aryl. In some embodiments, R₂ is substituted aryl. In some embodiments, R₂ is unsubstituted C₄-C₁₀heterocyclyl. In some embodiments, R₂ is substituted C₄-C₁₀heterocyclyl. In some embodiments, R₂ is unsubstituted heteroaryl. In some embodiments, R₂ is substituted heteroaryl. In some embodiments, R₂ is unsubstituted C₃-C₁₀cycloalkyl. In some embodiments, R₂ is substituted C₃-C₁₀cycloalkyl. In some embodiments, R₂ is —OPO₃WY. In some embodiments, R₂ is —OCH₂PO₄WY. In some embodiments, R₂ is —OCH₂PO₄Z. In some embodiments, R₂ is —OPO₃Z.

In some embodiments, R₇ is hydrogen. In some embodiments, R₇ is hydroxyl. In some embodiments, R₇ is carboxaldehyde. In some embodiments, R₇ is unsubstituted amine. In some embodiments, R₇ is substituted amine. In some embodiments, R₇ is unsubstituted C₁-C₁₀ alkyl. In some embodiments, R₇ is substituted C₁-C₁₀ alkyl. In some embodiments, R₇ is unsubstituted C₂-C₁₀ alkynyl. In some embodiments, R₇ is substituted C₂-C₁₀ alkynyl. In some embodiments, R₇ is unsubstituted C₂-C₁₀ alkenyl. In some embodiments, R₇ is substituted C₂-C₁₀ alkenyl. In some embodiments, R₇ is carboxyl. In some embodiments, R₇ is unsubstituted carbohydrate. In some embodiments, R₇ is substituted carbohydrate. In some embodiments, R₇ is unsubstituted ester. In some embodiments, R₇ is substituted ester. In some embodiments, R₇ is unsubstituted acyloxy. In some embodiments, R₇ is substituted acyloxy. In some embodiments, R₇ is nitro. In some embodiments, R₇ is halogen. In some embodiments, R₇ is unsubstituted C₁-C₁₀ aliphatic acyl. In some embodiments, R₇ is substituted C₁-C₁₀ aliphatic acyl. In some embodiments, R₇ is unsubstituted C₆-C₁₀ aromatic acyl. In some embodiments, R₇ is substituted C₆-C₁₀ aromatic acyl. In some embodiments, R₇ is unsubstituted C₆-C₁₀ aralkyl acyl. In some embodiments, R₇ is substituted C₆-C₁₀ aralkyl acyl. In some embodiments, R₇ is unsubstituted C₆-C₁₀ alkylaryl acyl. In some embodiments, R₇ is substituted C₆-C₁₀ alkylaryl acyl. In some embodiments, R₇ is unsubstituted alkoxy. In some embodiments, R₇ is substituted alkoxy. In some embodiments, R₇ is unsubstituted aryl. In some embodiments, R₇ is substituted aryl. In some embodiments, R₇ is unsubstituted C₃-C₁₀heterocyclyl. In some embodiments, R₇ is substituted C₃-C₁₀ heterocyclyl. In some embodiments, R₇ is unsubstituted heteroaryl, In some embodiments, R₇ is unsubstituted C₃-C₁₀cycloalkyl. In some embodiments, R₇ is substituted C₃-C₁₀cycloalkyl. In some embodiments, R₇ is —OPO₃WY. In some embodiments, R₇ is —OCH₂PO₄WY. In some embodiments, R₇ is —OCH₂PO₄Z. In some embodiments, R₇ is —OPO₃Z.

In some embodiments, R₉ is hydrogen. In some embodiments, R₉ is hydroxyl. In some embodiments, R₉ is carboxaldehyde. In some embodiments, R₉ is unsubstituted amine. In some embodiments, R₉ is substituted amine. In some embodiments, R₉ is unsubstituted C₁-C₁₀ alkyl. In some embodiments, R₉ is substituted C₁-C₁₀ alkyl. In some embodiments, R₉ is unsubstituted C₂-C₁₀ alkynyl. In some embodiments, R₉ is substituted C₂-C₁₀ alkynyl. In some embodiments, R₉ is unsubstituted C₂-C₁₀ alkenyl. In some embodiments, R₉ is substituted C₂-C₁₀ alkenyl. In some embodiments, R₉ is carboxyl. In some embodiments, R₉ is unsubstituted carbohydrate. In some embodiments, R₉ is substituted carbohydrate. In some embodiments, R₉ is unsubstituted ester. In some embodiments, R₉ is substituted ester. In some embodiments, R₉ is unsubstituted acyloxy. In some embodiments, R₉ is substituted acyloxy. In some embodiments, R₉ is nitro. In some embodiments, R₉ is halogen. In some embodiments, R₉ is unsubstituted C₁-C₁₀ aliphatic acyl. In some embodiments, R₉ is substituted C₁-C₁₀ aliphatic acyl. In some embodiments, R₉ is unsubstituted C₆-C₁₀ aromatic acyl. In some embodiments, R₉ is substituted C₆-C₁₀ aromatic acyl. In some embodiments, R₉ is unsubstituted C₆-C₁₀ aralkyl acyl. In some embodiments, R₉ is substituted C₆-C₁₀ aralkyl acyl. In some embodiments, R₉ is unsubstituted C₆-C₁₀ alkylaryl acyl. In some embodiments, R₉ is substituted C₆-C₁₀ alkylaryl acyl. In some embodiments, R₉ is unsubstituted alkoxy. In some embodiments, R₉ is substituted alkoxy. In some embodiments, R₉ is unsubstituted aryl. In some embodiments, R₉ is substituted aryl. In some embodiments, R₉ is unsubstituted C₃-C₁₀heterocyclyl. In some embodiments, R₉ is substituted C₃-C₁₀ heterocyclyl. In some embodiments, R₉ is unsubstituted heteroaryl, In some embodiments, R₉ is unsubstituted C₃-C₁₀cycloalkyl. In some embodiments, R₉ is substituted C₃-C₁₀cycloalkyl. In some embodiments, R₉ is —OPO₃WY. In some embodiments, R₉ is —OCH₂PO₄WY. In some embodiments, R₉ is —OCH₂PO₄Z. In some embodiments, R₉ is —OPO₃Z.

In some embodiments, R₁₆ is hydrogen. In some embodiments, R₁₆ is unsubstituted C₁-C₁₀ alkyl. In some embodiments, R₁₆ is substituted C₁-C₁₀ alkyl. In some embodiments, R₁₆ is unsubstituted C₂-C₁₀ alkynyl. In some embodiments, R₁₆ is substituted C₂-C₁₀ alkynyl. In some embodiments, R₁₆ is unsubstituted C₂-C₁₀ alkenyl. In some embodiments, R₁₆ is substituted C₂-C₁₀ alkenyl. In some embodiments, R₁₆ is unsubstituted carbohydrate. In some embodiments, R₁₆ is substituted carbohydrate. In some embodiments, R₁₆ is unsubstituted C₁-C₁₀ aliphatic acyl. In some embodiments, R₁₆ is substituted C₁-C₁₀ aliphatic acyl. In some embodiments, R₁₆ is unsubstituted C₆-C₁₀ aromatic acyl. In some embodiments, R₁₆ is substituted C₆-C₁₀ aromatic acyl. In some embodiments, R₁₆ is unsubstituted C₆-C₁₀ aralkyl acyl. In some embodiments, R₁₆ is substituted C₆-C₁₀ aralkyl acyl. In some embodiments, R₁₆ is unsubstituted C₆-C₁₀ alkylaryl acyl. In some embodiments, R₁₆ is substituted C₆-C₁₀ alkylaryl acyl. In some embodiments, R₁₆ is unsubstituted aryl. In some embodiments, R₁₆ is substituted aryl. In some embodiments, R₁₆ is unsubstituted C₃-C₁₀heterocyclyl. In some embodiments, R₁₆ is substituted C₃-C₁₀ heterocyclyl. In some embodiments, R₁₆ is unsubstituted heteroaryl. In some embodiments, R₁₆ is substituted heteroaryl. In some embodiments, R₁₆ is unsubstituted C₃-C₁₀cycloalkyl. In some embodiments, R₁₆ is substituted C₃-C₁₀cycloalkyl. In some embodiments, R₁₆ is —PO₃WY. In some embodiments, R₁₆ is —CH₂PO₄WY. In some embodiments, R₁₆ is —CH₂PO₄Z. In some embodiments, R₁₆ is —PO₃Z.

In some embodiments, R₁₈ is hydrogen. In some embodiments, R₁₈ is hydroxy. In some embodiments, R₁₈ is carboxaldehyde. In some embodiments, R₁₈ is unsubstituted amine. In some embodiments, R₁₈ is substituted amine. In some embodiments, R₁₈ is unsubstituted C₁-C₁₀ alkyl. In some embodiments, R₁₈ is unsubstituted C₂-C₁₀ alkynyl. In some embodiments, R₁₈ is substituted C₂-C₁₀ alkynyl. In some embodiments, R₁₈ is unsubstituted C₂-C₁₀ alkenyl. In some embodiments, R₁₈ is substituted C₂-C₁₀ alkenyl. In some embodiments, R₁₈ is carboxyl. In some embodiments, R₁₈ is unsubstituted carbohydrate. In some embodiments, R₁₈ is substituted carbohydrate. In some embodiments, R₁₈ is substituted carbohydrate. In some embodiments, R₁₈ is unsubstituted ester. In some embodiments, R₁₈ is substituted ester. In some embodiments, R₁₈ is unsubstituted acyloxy. In some embodiments, R₁₈ is substituted acyloxy. In some embodiments, R₁₈ is nitro. In some embodiments, R₁₈ is halogen. In some embodiments, R₁₈ is unsubstituted C₁-C₁₀ aliphatic acyl. In some embodiments, R₁₈ is substituted C₁-C₁₀ aliphatic acyl. In some embodiments, R₁₈ is unsubstituted C₆-C₁₀ aromatic acyl. In some embodiments, R₁₈ is substituted C₆-C₁₀ aromatic acyl. In some embodiments, R₁₈ is unsubstituted C₆-C₁₀ aralkyl acyl. In some embodiments, R₁₈ is substituted C₆-C₁₀ aralkyl acyl. In some embodiments, R₁₈ is unsubstituted C₆-C₁₀ alkylaryl acyl. In some embodiments, R₁₈ is substituted C₆-C₁₀ alkylaryl acyl. In some embodiments, R₁₈ is unsubstituted alkoxy. In some embodiments, R₁₈ is substituted alkoxy. In some embodiments, R₁₈ is unsubstituted aryl. In some embodiments, R₁₈ is substituted aryl. In some embodiments, R₁₈ is unsubstituted C₃-C₁₀heterocyclyl. In some embodiments, R₁₈ is substituted C₃-C₁₀heterocyclyl. In some embodiments, R₁₈ is unsubstituted heteroaryl. In some embodiments, R₁₈ is substituted heteroaryl. In some embodiments, R₁₈ is unsubstituted C₃-C₁₀cycloalkyl. In some embodiments, R₁₈ is substituted C₃-C₁₀cycloalkyl. In some embodiments, R₁₈ is —OPO₃WY. In some embodiments, R₁₈ is —OCH₂PO₄WY. In some embodiments, R₁₈ is —OCH₂PO₄Z. In some embodiments, R₁₈ is —OPO₃Z.

In some embodiments, R₁₉ is hydrogen. In some embodiments, R₁₉ is unsubstituted C₁-C₁₀ alkyl. In some embodiments, R₁₉ is substituted C₁-C₁₀ alkyl. In some embodiments, R₁₉ is unsubstituted C₂-C₁₀ alkynyl. In some embodiments, R₁₉ is substituted C₂-C₁₀ alkynyl. In some embodiments, R₁₉ is unsubstituted C₂-C₁₀ alkenyl. In some embodiments, R₁₉ is substituted C₂-C₁₀ alkenyl. In some embodiments, R₁₉ is unsubstituted carbohydrate. In some embodiments, R₁₉ is substituted carbohydrate. In some embodiments, R₁₉ is unsubstituted C₁-C₁₀ aliphatic acyl. In some embodiments, R₁₉ is substituted C₁-C₁₀ aliphatic acyl. In some embodiments, R₁₉ is unsubstituted C₆-C₁₀ aromatic acyl. In some embodiments, R₁₉ is substituted C₆-C₁₀ aromatic acyl. In some embodiments, R₁₉ is unsubstituted C₆-C₁₀ aralkyl acyl. In some embodiments, R₁₉ is substituted C₆-C₁₀ aralkyl acyl. In some embodiments, R₁₉ is unsubstituted C₆-C₁₀ alkylaryl acyl. In some embodiments, R₁₉ is substituted C₆-C₁₀ alkylaryl acyl. In some embodiments, R₁₉ is unsubstituted aryl. In some embodiments, R₁₉ is substituted aryl. In some embodiments, R₁₉ is unsubstituted C₃-C₁₀heterocyclyl. In some embodiments, R₁₉ is substituted C₃-C₁₀ heterocyclyl. In some embodiments, R₁₉ is unsubstituted heteroaryl. In some embodiments, R₁₉ is substituted heteroaryl. In some embodiments, R₁₉ is unsubstituted C₃-C₁₀cycloalkyl. In some embodiments, R₁₉ is substituted C₃-C₁₀cycloalkyl. In some embodiments, R₁₉ is —PO₃WY. In some embodiments, R₁₉ is —CH₂PO₄WY. In some embodiments, R₁₉ is —CH₂PO₄Z. In some embodiments, R₁₉ is —PO₃Z.

In some embodiments, s is an integer of 0. In some embodiments, s is an integer of 1. In some embodiments, s is an integer of 2. In some embodiments, s is an integer of 3.

In various embodiments, W is hydrogen. In various embodiments, W is unsubstituted methyl. In various embodiments, W is substituted methyl. In various embodiments, W is unsubstituted ethyl. In various embodiments, W is substituted ethyl. In various embodiments, W is unsubstituted alkyl. In various embodiments, W is substituted alkyl. In various embodiments, W is unsubstituted carbohydrate. In various embodiments, W is substituted carbohydrate. In various embodiments, W is potassium. In various embodiments, W is sodium. In various embodiments, W is lithium.

In various embodiments, Y is hydrogen. In various embodiments, Y is unsubstituted methyl. In various embodiments, Y is substituted methyl. In various embodiments, Y is unsubstituted ethyl. In various embodiments, Y is substituted ethyl. In various embodiments, Y is unsubstituted alkyl. In various embodiments, Y is substituted alkyl. In various embodiments, Y is unsubstituted carbohydrate. In various embodiments, Y is substituted carbohydrate. In various embodiments, Y is potassium. In various embodiments, Y is sodium. In various embodiments, Y is lithium.

In various embodiments, Z is calcium. In various embodiments, Z is magnesium. In various embodiments, Z is iron.

In other embodiments of the invention, the pyrone analogs of the invention are of Formula XI or a pharmaceutically/veterinarily acceptable salt thereof:

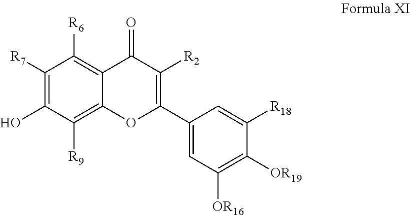

wherein R₂ is hydrogen, hydroxyl, C₁-C₁₀ alkyl, C₂-C₁₀ alkynyl, C₂-C₁₀ alkenyl, carboxyl, carbohydrate, ester, acyloxy, nitro, halogen, C₁-C₁₀ aliphatic acyl, C₆-C₁₀ aromatic acyl, C₆-C₁₀ aralkyl acyl, C₆-C₁₀ alkylaryl acyl, alkoxy, amine, aryl, C₄-C₁₀heterocyclyl, heteroaryl, C₃-C₁₀cycloalkyl, —OPO₃WY, —OCH₂PO₄WY, —OCH₂PO₄Z or —OPO₃Z;

R₆, R₇, and R₉ are independently hydrogen, hydroxyl, carboxaldehyde, amino, C₁-C₁₀ alkyl, C₂-C₁₀ alkynyl, C₂-C₁₀ alkenyl, carboxyl, carbohydrate, ester, acyloxy, nitro, halogen, C₁-C₁₀ aliphatic acyl, C₆-C₁₀ aromatic acyl, C₆-C₁₀ aralkyl acyl, C₆-C₁₀ alkylaryl acyl, alkoxy, amine, aryl, C₃-C₁₀ heterocyclyl, heteroaryl, C₃-C₁₀cycloalkyl, —OPO₃WY, —OCH₂PO₄WY, —OCH₂PO₄Z or —OPO₃Z;

wherein R₁₆ is hydrogen, C₁-C₁₀ alkyl, C₂-C₁₀ alkynyl, C₂-C₁₀ alkenyl, carbohydrate, C₁-C₁₀ aliphatic acyl, C₆-C₁₀ aromatic acyl, C₆-C₁₀ aralkyl acyl, C₆-C₁₀ alkylaryl acyl, aryl, C₃-C₁₀ heterocyclyl, heteroaryl, C₃-C₁₀cycloalkyl, —PO₃WY, —CH₂PO₄WY, —CH₂PO₄Z or —PO₃Z;

each instance of R₁₈ is independently hydrogen, hydroxyl, carboxaldehyde, amine, C₁-C₁₀ alkyl, C₂-C₁₀ alkynyl, C₂-C₁₀ alkenyl, carboxyl, carbohydrate, ester, acyloxy, nitro, halogen, C₁-C₁₀ aliphatic acyl, C₆-C₁₀ aromatic acyl, C₆-C₁₀ aralkyl acyl, C₆-C₁₀alkylaryl acyl, alkoxy, alkyl, phosphate, aryl, heteroaryl, C₃-C₁₀ heterocyclic, C₃-C₁₀cycloalkyl, —OPO₃WY, —OCH₂PO₄WY, —OCH₂PO₄Z or —OPO₃Z;

R₁₉ is hydrogen, C₁-C₁₀ alkyl, C₂-C₁₀ alkynyl, C₂-C₁₀ alkenyl, carbohydrate, C₁-C₁₀ aliphatic acyl, C₆-C₁₀ aromatic acyl, C₆-C₁₀ aralkyl acyl, C₆-C₁₀ alkylaryl acyl, aryl, C₃-C₁₀heterocyclyl, heteroaryl, optionally substituted C₃-C₁₀cycloalkyl, —PO₃WY, —CH₂PO₄WY, —CH₂PO₄Z or —PO₃Z;

s is an integer of 0, 1, 2, or 3; and

W and Y are independently hydrogen, methyl, ethyl, alkyl, carbohydrate, or a cation, and Z is a multivalent cation; and

wherein the compound of Formula XI is not the compound of Formula A:

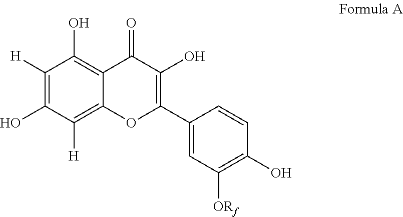

wherein R_{f} is hydrogen; and

wherein the compound of Formula XI is not the compound wherein:

R₂ is hydrogen, R₆ is hydroxy, R₇ and R₉ are hydrogen, R₁₆ is hydrogen, R₁₈ is hydrogen, and R₁₉ is hydrogen;

R₂ is hydrogen, R₆ is hydroxy, R₇ and R₉ are hydrogen, R₁₆ is hydrogen, R₁₉ is hydrogen, and R₁₈ is hydroxy;

R₂ is hydrogen, R₆ is hydroxy, R₇ and R₉ are hydrogen, R₁₆ is hydrogen, R₁₉ is hydrogen, and R₁₈ is methoxy;

R₂ is hydroxy, R₇ and R₉ are hydrogen, R₁₆ is methyl, R₁₉ is hydrogen, and R₁₈ is hydrogen;

R₂ is hydroxy, R₇ and R₉ are hydrogen, R₁₆ is hydrogen, R₁₉ is methyl, and R₁₈ is hydrogen;

R₂ is hydrogen, R₆, R₇, and R₉ are hydrogen, R₁₆ is hydrogen, R₁₉ is hydrogen, and R₁₈ is hydrogen;

R₂ is hydrogen, R₆, R₇, and R₉ are hydrogen, R₁₆ is hydrogen, R₁₉ is hydrogen, and R₁₈ is hydroxy;

R₂ is hydroxy, R₆, R₇, and R₉ are hydrogen, R₁₆ is hydrogen, R₁₉ is hydrogen, and R₁₈ is hydrogen; or

R₂ is hydroxy, R₆, R₇, and R₉ are hydrogen, R₁₆ is hydrogen, R₁₉ is hydrogen, and R₁₈ is hydroxy.

In some embodiments, the compound of Formula XI is not the compound of Formula A wherein R_{f} is PO₃K₂.

In some embodiments of the invention, at least one of R₁₆ and R₁₉ is —PO₃WY, —CH₂PO₄WY, —CH₂PO₄Z or —PO₃Z, or at least one of R₁₇ or R₁₈ is —OPO₃WY, —OCH₂PO₄WY, —OCH₂PO₄Z or —OPO₃Z. In some embodiments, at least one of R₁₆ and R₁₉ is —PO₃WY, —CH₂PO₄WY, —CH₂PO₄Z or —PO₃Z and at least one of R₁₇ or R₁₈ is —OPO₃WY, —OCH₂PO₄WY, —OCH₂PO₄Z or —OPO₃Z.

In some embodiments, R₂ is hydrogen. In some embodiments, R₂ is hydroxyl. In some embodiments, R₂ is optionally substituted C₁-C₁₀ alkyl. In some embodiments, R₂ is unsubstituted C₁-C₁₀ alkyl. In some embodiments, R₂ is substituted C₁-C₁₀ alkyl. In some embodiments, R₂ is unsubstituted C₁-C₁₀ alkyl. In some other embodiments, R₂ is substituted C₁-C₁₀ alkyl. In some embodiments, R₂ is unsubstituted C₂-C₁₀ alkynyl. In some embodiments, R₂ is substituted C₂-C₁₀ alkynyl. In some embodiments, R₂ is unsubstituted C₂-C₁₀ alkenyl. In some embodiments, R₂ is substituted C₂-C₁₀ alkenyl. In some embodiments, R₂ is carboxyl. In some embodiments, R₂ is unsubstituted carbohydrate. In some embodiments, R₂ is substituted carbohydrate. In some embodiments, R₂ is unsubstituted ester. In some embodiments, R₂ is substituted ester. In some embodiments, R₂ is unsubstituted acyloxy. In some embodiments, R₂ is substituted acyloxy. In some embodiments, R₂ is nitro. In some embodiments, R₂ is halogen. In some embodiments, R₂ is unsubstituted C₁-C₁₀ aliphatic acyl. In some embodiments, R₂ is substituted C₁-C₁₀ aliphatic acyl. In some embodiments, R₂ is unsubstituted C₆-C₁₀ aromatic acyl. In some embodiments, R₂ is substituted C₆-C₁₀ aromatic acyl. In some embodiments, R₂ is unsubstituted C₆-C₁₀ aralkyl acyl. In some embodiments, R₂ is substituted C₆-C₁₀ aralkyl acyl. In some embodiments, R₂ is unsubstituted C₆-C₁₀ alkylaryl acyl. In some embodiments, R₂ is substituted C₆-C₁₀ alkylaryl acyl. In some embodiments, R₂ is unsubstituted alkoxy. In some embodiments, R₂ is substituted alkoxy. In some embodiments, R₂ is unsubstituted amine. In some embodiments, R₂ is substituted amine. In some embodiments, R₂ is unsubstituted aryl. In some embodiments, R₂ is substituted aryl. In some embodiments, R₂ is unsubstituted C₄-C₁₀heterocyclyl. In some embodiments, R₂ is substituted C₄-C₁₀heterocyclyl. In some embodiments, R₂ is unsubstituted heteroaryl. In some embodiments, R₂ is substituted heteroaryl. In some embodiments, R₂ is unsubstituted C₃-C₁₀cycloalkyl. In some embodiments, R₂ is substituted C₃-C₁₀cycloalkyl. In some embodiments, R₂ is —OPO₃WY. In some embodiments, R₂ is —OCH₂PO₄WY. In some embodiments, R₂ is —OCH₂PO₄Z. In some embodiments, R₂ is —OPO₃Z.

In some embodiments, R₆ is hydrogen. In some embodiments, R₆ is hydroxyl. In some embodiments, R₆ is carboxaldehyde. In some embodiments, R₆ is unsubstituted amine. In some embodiments, R₆ is substituted amine. In some embodiments, R₆ is unsubstituted C₁-C₁₀ alkyl. In some embodiments, R₆ is substituted C₁-C₁₀ alkyl. In some embodiments, R₆ is unsubstituted C₂-C₁₀ alkynyl. In some embodiments, R₆ is substituted C₂-C₁₀ alkynyl. In some embodiments, R₆ is unsubstituted C₂-C₁₀ alkenyl. In some embodiments, R₆ is substituted C₂-C₁₀ alkenyl. In some embodiments, R₆ is carboxyl. In some embodiments, R₆ is unsubstituted carbohydrate. In some embodiments, R₆ is substituted carbohydrate. In some embodiments, R₆ is unsubstituted ester. In some embodiments, R₆ is substituted ester. In some embodiments, R₆ is unsubstituted acyloxy. In some embodiments, R₆ is substituted acyloxy. In some embodiments, R₆ is nitro. In some embodiments, R₆ is halogen. In some embodiments, R₆ is unsubstituted C₁-C₁₀ aliphatic acyl. In some embodiments, R₆ is substituted C₁-C₁₀ aliphatic acyl. In some embodiments, R₆ is unsubstituted C₆-C₁₀ aromatic acyl. In some embodiments, R₆ is substituted C₆-C₁₀ aromatic acyl. In some embodiments, R₆ is unsubstituted C₆-C₁₀ aralkyl acyl. In some embodiments, R₆ is substituted C₆-C₁₀ aralkyl acyl. In some embodiments, R₆ is unsubstituted C₆-C₁₀ alkylaryl acyl. In some embodiments, R₆ is substituted C₆-C₁₀ alkylaryl acyl. In some embodiments, R₆ is unsubstituted alkoxy. In some embodiments, R₆ is substituted alkoxy. In some embodiments, R₆ is unsubstituted aryl. In some embodiments, R₆ is substituted aryl. In some embodiments, R₆ is unsubstituted C₃-C₁₀heterocyclyl. In some embodiments, R₆ is substituted C₃-C₁₀ heterocyclyl. In some embodiments, R₆ is unsubstituted heteroaryl. In some embodiments, R₆ is unsubstituted C₃-C₁₀cycloalkyl. In some embodiments, R₆ is substituted C₃-C₁₀cycloalkyl. In some embodiments, R₆ is —OPO₃WY. In some embodiments, R₆ is —OCH₂PO₄WY. In some embodiments, R₆ is —OCH₂PO₄Z. In some embodiments, R₆ is —OPO₃Z.

In some embodiments, R₇ is hydrogen. In some embodiments, R₇ is hydroxyl. In some embodiments, R₇ is carboxaldehyde. In some embodiments, R₇ is unsubstituted amine. In some embodiments, R₇ is substituted amine. In some embodiments, R₇ is unsubstituted C₁-C₁₀ alkyl. In some embodiments, R₇ is substituted C₁-C₁₀ alkyl. In some embodiments, R₇ is unsubstituted C₂-C₁₀ alkynyl. In some embodiments, R₇ is substituted C₂-C₁₀ alkynyl. In some embodiments, R₇ is unsubstituted C₂-C₁₀ alkenyl. In some embodiments, R₇ is substituted C₂-C₁₀ alkenyl. In some embodiments, R₇ is carboxyl. In some embodiments, R₇ is unsubstituted carbohydrate. In some embodiments, R₇ is substituted carbohydrate. In some embodiments, R₇ is unsubstituted ester. In some embodiments, R₇ is substituted ester. In some embodiments, R₇ is unsubstituted acyloxy. In some embodiments, R₇ is substituted acyloxy. In some embodiments, R₇ is nitro. In some embodiments, R₇ is halogen. In some embodiments, R₇ is unsubstituted C₁-C₁₀ aliphatic acyl. In some embodiments, R₇ is substituted C₁-C₁₀ aliphatic acyl. In some embodiments, R₇ is unsubstituted C₆-C₁₀ aromatic acyl. In some embodiments, R₇ is substituted C₆-C₁₀ aromatic acyl. In some embodiments, R₇ is unsubstituted C₆-C₁₀ aralkyl acyl. In some embodiments, R₇ is substituted C₆-C₁₀ aralkyl acyl. In some embodiments, R₇ is unsubstituted C₆-C₁₀ alkylaryl acyl. In some embodiments, R₇ is substituted C₆-C₁₀ alkylaryl acyl. In some embodiments, R₇ is unsubstituted alkoxy. In some embodiments, R₇ is substituted alkoxy. In some embodiments, R₇ is unsubstituted aryl. In some embodiments, R₇ is substituted aryl. In some embodiments, R₇ is unsubstituted C₃-C₁₀heterocyclyl. In some embodiments, R₇ is substituted C₃-C₁₀ heterocyclyl. In some embodiments, R₇ is unsubstituted heteroaryl, In some embodiments, R₇ is unsubstituted C₃-C₁₀cycloalkyl. In some embodiments, R₇ is substituted C₃-C₁₀cycloalkyl. In some embodiments, R₇ is —OPO₃WY. In some embodiments, R₇ is —OCH₂PO₄WY. In some embodiments, R₇ is —OCH₂PO₄Z. In some embodiments, R₇ is —OPO₃Z.

In some embodiments, R₉ is hydrogen. In some embodiments, R₉ is hydroxyl. In some embodiments, R₉ is carboxaldehyde. In some embodiments, R₉ is unsubstituted amine. In some embodiments, R₉ is substituted amine. In some embodiments, R₉ is unsubstituted C₁-C₁₀ alkyl. In some embodiments, R₉ is substituted C₁-C₁₀ alkyl. In some embodiments, R₉ is unsubstituted C₂-C₁₀ alkynyl. In some embodiments, R₉ is substituted C₂-C₁₀ alkynyl. In some embodiments, R₉ is unsubstituted C₂-C₁₀ alkenyl. In some embodiments, R₉ is substituted C₂-C₁₀ alkenyl. In some embodiments, R₉ is carboxyl. In some embodiments, R₉ is unsubstituted carbohydrate. In some embodiments, R₉ is substituted carbohydrate. In some embodiments, R₉ is unsubstituted ester. In some embodiments, R₉ is substituted ester. In some embodiments, R₉ is unsubstituted acyloxy. In some embodiments, R₉ is substituted acyloxy. In some embodiments, R₉ is nitro. In some embodiments, R₉ is halogen. In some embodiments, R₉ is unsubstituted C₁-C₁₀ aliphatic acyl. In some embodiments, R₉ is substituted C₁-C₁₀ aliphatic acyl. In some embodiments, R₉ is unsubstituted C₆-C₁₁ aromatic acyl. In some embodiments, R₉ is substituted C₆-C₁ aromatic acyl. In some embodiments, R₉ is unsubstituted C₆-C₁₁ aralkyl acyl. In some embodiments, R₉ is substituted C₆-C₁₀ aralkyl acyl. In some embodiments, R₉ is unsubstituted C₆-C₁₀ alkylaryl acyl. In some embodiments, R₉ is substituted C₆-C₁₀ alkylaryl acyl. In some embodiments, R₉ is unsubstituted alkoxy. In some embodiments, R₉ is substituted alkoxy. In some embodiments, R₉ is unsubstituted aryl. In some embodiments, R₉ is substituted aryl. In some embodiments, R₉ is unsubstituted C₃-C₁₀heterocyclyl. In some embodiments, R₉ is substituted C₃-C₁₀ heterocyclyl. In some embodiments, R₉ is unsubstituted heteroaryl, In some embodiments, R₉ is unsubstituted C₃-C₁₀cycloalkyl. In some embodiments, R₉ is substituted C₃-C₁₀cycloalkyl. In some embodiments, R₉ is —OPO₃WY. In some embodiments, R₉ is —OCH₂PO₄WY. In some embodiments, R₉ is —OCH₂PO₄Z. In some embodiments, R₉ is —OPO₃Z.

In some embodiments, R₁₆ is hydrogen. In some embodiments, R₁₆ is unsubstituted C₁-C₁₀ alkyl. In some embodiments, R₁₆ is substituted C₁-C₁₀ alkyl. In some embodiments, R₁₆ is unsubstituted C₂-C₁₀ alkynyl. In some embodiments, R₁₆ is substituted C₂-C₁₀ alkynyl. In some embodiments, R₁₆ is unsubstituted C₂-C₁₀ alkenyl. In some embodiments, R₁₆ is substituted C₂-C₁₀ alkenyl. In some embodiments, R₁₆ is unsubstituted carbohydrate. In some embodiments, R₁₆ is substituted carbohydrate. In some embodiments, R₁₆ is unsubstituted C₁-C₁₀ aliphatic acyl. In some embodiments, R₁₆ is substituted C₁-C₁₀ aliphatic acyl. In some embodiments, R₁₆ is unsubstituted C₆-C₁₀ aromatic acyl. In some embodiments, R₁₆ is substituted C₆-C₁₀ aromatic acyl. In some embodiments, R₁₆ is unsubstituted C₆-C₁₀ aralkyl acyl. In some embodiments, R₁₆ is substituted C₆-C₁₀ aralkyl acyl. In some embodiments, R₁₆ is unsubstituted C₆-C₁₀ alkylaryl acyl. In some embodiments, R₁₆ is substituted C₆-C₁₀ alkylaryl acyl. In some embodiments, R₁₆ is unsubstituted aryl. In some embodiments, R₁₆ is substituted aryl. In some embodiments, R₁₆ is unsubstituted C₃-C₁₀heterocyclyl. In some embodiments, R₁₆ is substituted C₃-C₁₀ heterocyclyl. In some embodiments, R₁₆ is unsubstituted heteroaryl. In some embodiments, R₁₆ is substituted heteroaryl. In some embodiments, R₁₆ is unsubstituted C₃-C₁₀cycloalkyl. In some embodiments, R₁₆ is substituted C₃-C₁₀cycloalkyl. In some embodiments, R₁₆ is —PO₃WY. In some embodiments, R₁₆ is —CH₂PO₄WY. In some embodiments, R₁₆ is —CH₂PO₄Z. In some embodiments, R₁₆ is —PO₃Z.

In some embodiments, R₁₈ is hydrogen. In some embodiments, R₁₈ is hydroxy. In some embodiments, R₁₈ is carboxaldehyde. In some embodiments, R₁₈ is unsubstituted amine. In some embodiments, R₁₈ is substituted amine. In some embodiments, R₁₈ is unsubstituted C₁-C₁₀ alkyl. In some embodiments, R₁₈ is unsubstituted C₂-C₁₀ alkynyl. In some embodiments, R₁₈ is substituted C₂-C₁₀ alkynyl. In some embodiments, R₁₈ is unsubstituted C₂-C₁₀ alkenyl. In some embodiments, R₁₈ is substituted C₂-C₁₀ alkenyl. In some embodiments, R₁₈ is carboxyl. In some embodiments, R₁₈ is unsubstituted carbohydrate. In some embodiments, R₁₈ is substituted carbohydrate. In some embodiments, R₁₈ is substituted carbohydrate. In some embodiments, R₁₈ is unsubstituted ester. In some embodiments, R₁₈ is substituted ester. In some embodiments, R₁₈ is unsubstituted acyloxy. In some embodiments, R₁₈ is substituted acyloxy. In some embodiments, R₁₈ is nitro. In some embodiments, R₁₈ is halogen. In some embodiments, R₁₈ is unsubstituted C₁-C₁₀ aliphatic acyl. In some embodiments, R₁₈ is substituted C₁-C₁₀ aliphatic acyl. In some embodiments, R₁₈ is unsubstituted C₆-C₁₀ aromatic acyl. In some embodiments, R₁₈ is substituted C₆-C₁₀ aromatic acyl. In some embodiments, R₁₈ is unsubstituted C₆-C₁₀ aralkyl acyl. In some embodiments, R₁₈ is substituted C₆-C₁₀ aralkyl acyl. In some embodiments, R₁₈ is unsubstituted C₆-C₁₀ alkylaryl acyl. In some embodiments, R₁₈ is substituted C₆-C₁₀ alkylaryl acyl. In some embodiments, R₁₈ is unsubstituted alkoxy. In some embodiments, R₁₈ is substituted alkoxy. In some embodiments, R₁₈ is unsubstituted aryl. In some embodiments, R₁₈ is substituted aryl. In some embodiments, R₁₈ is unsubstituted C₃-C₁₀heterocyclyl. In some embodiments, R₁₈ is substituted C₃-C₁₀heterocyclyl. In some embodiments, R₁₈ is unsubstituted heteroaryl. In some embodiments, R₁₈ is substituted heteroaryl. In some embodiments, R₁₈ is unsubstituted C₃-C₁₀cycloalkyl. In some embodiments, R₁₈ is substituted C₃-C₁₀cycloalkyl. In some embodiments, R₁₈ is —OPO₃WY. In some embodiments, R₁₈ is —OCH₂PO₄WY. In some embodiments, R₁₈ is —OCH₂PO₄Z. In some embodiments, R₁₈ is —OPO₃Z.

In some embodiments, R₁₉ is hydrogen. In some embodiments, R₁₉ is unsubstituted C₁-C₁₀ alkyl. In some embodiments, R₁₉ is substituted C₁-C₁₀ alkyl. In some embodiments, R₁₉ is unsubstituted C₂-C₁₀ alkynyl. In some embodiments, R₁₉ is substituted C₂-C₁₀ alkynyl. In some embodiments, R₁₉ is unsubstituted C₂-C₁₀ alkenyl. In some embodiments, R₁₉ is substituted C₂-C₁₀ alkenyl. In some embodiments, R₁₉ is unsubstituted carbohydrate. In some embodiments, R₁₉ is substituted carbohydrate. In some embodiments, R₁₉ is unsubstituted C₁-C₁₀ aliphatic acyl. In some embodiments, R₁₉ is substituted C₁-C₁₀ aliphatic acyl. In some embodiments, R₁₉ is unsubstituted C₆-C₁₀ aromatic acyl. In some embodiments, R₁₉ is substituted C₆-C₁₀ aromatic acyl. In some embodiments, R₁₉ is unsubstituted C₆-C₁₀ aralkyl acyl. In some embodiments, R₁₉ is substituted C₆-C₁₀ aralkyl acyl. In some embodiments, R₁₉ is unsubstituted C₆-C₁₀ alkylaryl acyl. In some embodiments, R₁₉ is substituted C₆-C₁₀ alkylaryl acyl. In some embodiments, R₁₉ is unsubstituted aryl. In some embodiments, R₁₉ is substituted aryl. In some embodiments, R₁₉ is unsubstituted C₃-C₁₀heterocyclyl. In some embodiments, R₁₉ is substituted C₃-C₁₀ heterocyclyl. In some embodiments, R₁₉ is unsubstituted heteroaryl. In some embodiments, R₁₉ is substituted heteroaryl. In some embodiments, R₁₉ is unsubstituted C₃-C₁₀cycloalkyl. In some embodiments, R₁₉ is substituted C₃-C₁₀cycloalkyl. In some embodiments, R₁₉ is —PO₃WY. In some embodiments, R₁₉ is —CH₂PO₄WY. In some embodiments, R₁₉ is —CH₂PO₄Z. In some embodiments, R₁₉ is —PO₃Z.

In some embodiments, s is an integer of 0. In some embodiments, s is an integer of 1. In some embodiments, s is an integer of 2. In some embodiments, s is an integer of 3.

In various embodiments, W is hydrogen. In various embodiments, W is unsubstituted methyl. In various embodiments, W is substituted methyl. In various embodiments, W is unsubstituted ethyl. In various embodiments, W is substituted ethyl. In various embodiments, W is unsubstituted alkyl. In various embodiments, W is substituted alkyl. In various embodiments, W is unsubstituted carbohydrate. In various embodiments, W is substituted carbohydrate. In various embodiments, W is potassium. In various embodiments, W is sodium. In various embodiments, W is lithium.

In various embodiments, Y is hydrogen. In various embodiments, Y is unsubstituted methyl. In various embodiments, Y is substituted methyl. In various embodiments, Y is unsubstituted ethyl. In various embodiments, Y is substituted ethyl. In various embodiments, Y is unsubstituted alkyl. In various embodiments, Y is substituted alkyl. In various embodiments, Y is unsubstituted carbohydrate. In various embodiments, Y is substituted carbohydrate. In various embodiments, Y is potassium. In various embodiments, Y is sodium. In various embodiments, Y is lithium.

In various embodiments, Z is calcium. In various embodiments, Z is magnesium. In various embodiments, Z is iron.

In other embodiments of the invention, the pyrone analog is of Formula XII or a pharmaceutically/veterinarily acceptable salt thereof:

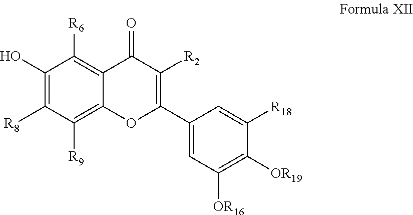

wherein R₂ is hydrogen, hydroxyl, C₁-C₁₀ alkyl, C₂-C₁₀ alkynyl, C₂-C₁₀ alkenyl, carboxyl, carbohydrate, ester, acyloxy, nitro, halogen, C₁-C₁₀ aliphatic acyl, C₆-C₁₀ aromatic acyl, C₆-C₁₀ aralkyl acyl, C₆-C₁₀ alkylaryl acyl, alkoxy, amine, aryl, C₄-C₁₀heterocyclyl, heteroaryl, C₃-C₁₀cycloalkyl, —OPO₃WY, —OCH₂PO₄WY, —OCH₂PO₄Z or —OPO₃Z;

R₆, R₈, and R₉ are independently hydrogen, hydroxyl, carboxaldehyde, amino, C₁-C₁₀ alkyl, C₂-C₁₀ alkynyl, C₂-C₁₀ alkenyl, carboxyl, carbohydrate, ester, acyloxy, nitro, halogen, C₁-C₁₀ aliphatic acyl, C₆-C₁₀ aromatic acyl, C₆-C₁₀ aralkyl acyl, C₆-C₁₀ alkylaryl acyl, alkoxy, amine, aryl, C₃-C₁₀ heterocyclyl, heteroaryl, C₃-C₁₀cycloalkyl, —OPO₃WY, —OCH₂PO₄WY, —OCH₂PO₄Z or —OPO₃Z;

wherein R₁₆ is hydrogen, C₁-C₁₀ alkyl, C₂-C₁₀ alkynyl, C₂-C₁₀ alkenyl, carbohydrate, C₁-C₁₀ aliphatic acyl, C₆-C₁₀ aromatic acyl, C₆-C₁₀ aralkyl acyl, C₆-C₁₀ alkylaryl acyl, aryl, C₃-C₁₀ heterocyclyl, heteroaryl, C₃-C₁₀cycloalkyl, —PO₃WY, —CH₂PO₄WY, —CH₂PO₄Z or —PO₃Z;

each instance of R₁₈ is independently hydrogen, hydroxyl, carboxaldehyde, amine, C₁-C₁₀ alkyl, C₂-C₁₀ alkynyl, C₂-C₁₀ alkenyl, carboxyl, carbohydrate, ester, acyloxy, nitro, halogen, C₁-C₁₀ aliphatic acyl, C₆-C₁₀ aromatic acyl, C₆-C₁₀ aralkyl acyl, C₆-C₁₀alkylaryl acyl, alkoxy, alkyl, phosphate, aryl, heteroaryl, C₃-C₁₀ heterocyclic, C₃-C₁₀cycloalkyl, —OPO₃WY, —OCH₂PO₄WY, —OCH₂PO₄Z or —OPO₃Z;

R₁₉ is hydrogen, C₁-C₁₀ alkyl, C₂-C₁₀ alkynyl, C₂-C₁₀ alkenyl, carbohydrate, C₁-C₁₀ aliphatic acyl, C₆-C₁₀ aromatic acyl, C₆-C₁₀ aralkyl acyl, C₆-C₁₀ alkylaryl acyl, aryl, C₃-C₁₀heterocyclyl, heteroaryl, optionally substituted C₃-C₁₀cycloalkyl, —PO₃WY, —CH₂PO₄WY, —CH₂PO₄Z or —PO₃Z;

s is an integer of 0, 1, 2, or 3;

W and Y are independently hydrogen, methyl, ethyl, alkyl, carbohydrate, or a cation, and Z is a multivalent cation; and

wherein the compound of Formula XII is not the compound wherein

R₂ is hydrogen, R₆, R₈, and R₉ are hydrogen, R₁₆ is hydrogen, R₁₉ is hydrogen, and R₁₈ is hydrogen;

R₂ is hydrogen, R₆, R₈, and R₉ are hydrogen, R₁₆ is hydrogen, R₁₉ is hydrogen, and R₁₈ is hydroxy; or

R₂ is hydroxy, R₆, R₈, and R₉ are hydrogen, R₁₆ is hydrogen, R₁₉ is hydrogen, and R₁₈ is hydrogen.

In some embodiments of the invention, at least one of R₁₆ and R₁₉ is —PO₃WY, —CH₂PO₄WY, —CH₂PO₄Z or —PO₃Z, or at least one of R₁₇ or R₁₈ is —OPO₃WY, —OCH₂PO₄WY, —OCH₂PO₄Z or —OPO₃Z. In some embodiments, at least one of R₁₆ and R₁₉ is —PO₃WY, —CH₂PO₄WY, —CH₂PO₄Z or —PO₃Z and at least one of R₁₇ or R₁₈ is —OPO₃WY, —OCH₂PO₄WY, —OCH₂PO₄Z or —OPO₃Z.

In some embodiments, R₂ is hydrogen. In some embodiments, R₂ is hydroxyl. In some embodiments, R₂ is optionally substituted C₁-C₁₀ alkyl. In some embodiments, R₂ is unsubstituted C₁-C₁₀ alkyl. In some embodiments, R₂ is substituted C₁-C₁₀ alkyl. In some embodiments, R₂ is unsubstituted C₁-C₁₀ alkyl. In some other embodiments, R₂ is substituted C₁-C₁₀ alkyl. In some embodiments, R₂ is unsubstituted C₂-C₁₀ alkynyl. In some embodiments, R₂ is substituted C₂-C₁₀ alkynyl. In some embodiments, R₂ is unsubstituted C₂-C₁₀ alkenyl. In some embodiments, R₂ is substituted C₂-C₁₀ alkenyl. In some embodiments, R₂ is carboxyl. In some embodiments, R₂ is unsubstituted carbohydrate. In some embodiments, R₂ is substituted carbohydrate. In some embodiments, R₂ is unsubstituted ester. In some embodiments, R₂ is substituted ester. In some embodiments, R₂ is unsubstituted acyloxy. In some embodiments, R₂ is substituted acyloxy. In some embodiments, R₂ is nitro. In some embodiments, R₂ is halogen. In some embodiments, R₂ is unsubstituted C₁-C₁₀ aliphatic acyl. In some embodiments, R₂ is substituted C₁-C₁₀ aliphatic acyl. In some embodiments, R₂ is unsubstituted C₆-C₁₀ aromatic acyl. In some embodiments, R₂ is substituted C₆-C₁₀ aromatic acyl. In some embodiments, R₂ is unsubstituted C₆-C₁₀ aralkyl acyl. In some embodiments, R₂ is substituted C₆-C₁₀ aralkyl acyl. In some embodiments, R₂ is unsubstituted C₆-C₁₀ alkylaryl acyl. In some embodiments, R₂ is substituted C₆-C₁₀ alkylaryl acyl. In some embodiments, R₂ is unsubstituted alkoxy. In some embodiments, R₂ is substituted alkoxy. In some embodiments, R₂ is unsubstituted amine. In some embodiments, R₂ is substituted amine. In some embodiments, R₂ is unsubstituted aryl. In some embodiments, R₂ is substituted aryl. In some embodiments, R₂ is unsubstituted C₄-C₁₀heterocyclyl. In some embodiments, R₂ is substituted C₄-C₁₀heterocyclyl. In some embodiments, R₂ is unsubstituted heteroaryl. In some embodiments, R₂ is substituted heteroaryl. In some embodiments, R₂ is unsubstituted C₃-C₁₀cycloalkyl. In some embodiments, R₂ is substituted C₃-C₁₀cycloalkyl. In some embodiments, R₂ is —OPO₃WY. In some embodiments, R₂ is —OCH₂PO₄WY. In some embodiments, R₂ is —OCH₂PO₄Z. In some embodiments, R₂ is —OPO₃Z.

In some embodiments, R₆ is hydrogen. In some embodiments, R₆ is hydroxyl. In some embodiments, R₆ is carboxaldehyde. In some embodiments, R₆ is unsubstituted amine. In some embodiments, R₆ is substituted amine. In some embodiments, R₆ is unsubstituted C₁-C₁₀ alkyl. In some embodiments, R₆ is substituted C₁-C₁₀ alkyl. In some embodiments, R₆ is unsubstituted C₂-C₁₀ alkynyl. In some embodiments, R₆ is substituted C₂-C₁₀ alkynyl. In some embodiments, R₆ is unsubstituted C₂-C₁₀ alkenyl. In some embodiments, R₆ is substituted C₂-C₁₀ alkenyl. In some embodiments, R₆ is carboxyl. In some embodiments, R₆ is unsubstituted carbohydrate. In some embodiments, R₆ is substituted carbohydrate. In some embodiments, R₆ is unsubstituted ester. In some embodiments, R₆ is substituted ester. In some embodiments, R₆ is unsubstituted acyloxy. In some embodiments, R₆ is substituted acyloxy. In some embodiments, R₆ is nitro. In some embodiments, R₆ is halogen. In some embodiments, R₆ is unsubstituted C₁-C₁₀ aliphatic acyl. In some embodiments, R₆ is substituted C₁-C₁₀ aliphatic acyl. In some embodiments, R₆ is unsubstituted C₆-C₁₀ aromatic acyl. In some embodiments, R₆ is substituted C₆-C₁₀ aromatic acyl. In some embodiments, R₆ is unsubstituted C₆-C₁₀ aralkyl acyl. In some embodiments, R₆ is substituted C₆-C₁₀ aralkyl acyl. In some embodiments, R₆ is unsubstituted C₆-C₁₀ alkylaryl acyl. In some embodiments, R₆ is substituted C₆-C₁₀ alkylaryl acyl. In some embodiments, R₆ is unsubstituted alkoxy. In some embodiments, R₆ is substituted alkoxy. In some embodiments, R₆ is unsubstituted aryl. In some embodiments, R₆ is substituted aryl. In some embodiments, R₆ is unsubstituted C₃-C₁₀heterocyclyl. In some embodiments, R₆ is substituted C₃-C₁₀ heterocyclyl. In some embodiments, R₆ is unsubstituted heteroaryl, In some embodiments, R₆ is unsubstituted C₃-C₁₀cycloalkyl. In some embodiments, R₆ is substituted C₃-C₁₀cycloalkyl. In some embodiments, R₆ is —OPO₃WY. In some embodiments, R₆ is —OCH₂PO₄WY. In some embodiments, R₆ is —OCH₂PO₄Z. In some embodiments, R₆ is —OPO₃Z.

In some embodiments, R₈ is hydrogen. In some embodiments, R₈ is hydroxyl. In some embodiments, R₈ is carboxaldehyde. In some embodiments, R₈ is unsubstituted amine. In some embodiments, R₈ is substituted amine. In some embodiments, R₈ is unsubstituted C₁-C₁₀ alkyl. In some embodiments, R₈ is substituted C₁-C₁₀ alkyl. In some embodiments, R₈ is unsubstituted C₂-C₁₀ alkynyl. In some embodiments, R₈ is substituted C₂-C₁₀ alkynyl. In some embodiments, R₈ is unsubstituted C₂-C₁₀ alkenyl. In some embodiments, R₈ is substituted C₂-C₁₀ alkenyl. In some embodiments, R₈ is carboxyl. In some embodiments, R₈ is unsubstituted carbohydrate. In some embodiments, R₈ is substituted carbohydrate. In some embodiments, R₈ is unsubstituted ester. In some embodiments, R₈ is substituted ester. In some embodiments, R₈ is unsubstituted acyloxy. In some embodiments, R₈ is substituted acyloxy. In some embodiments, R₈ is nitro. In some embodiments, R₈ is halogen. In some embodiments, R₈ is unsubstituted C₁-C₁₀ aliphatic acyl. In some embodiments, R₈ is substituted C₁-C₁₀ aliphatic acyl. In some embodiments, R₈ is unsubstituted C₆-C₁₀ aromatic acyl. In some embodiments, R₈ is substituted C₆-C₁₀ aromatic acyl. In some embodiments, R₈ is unsubstituted C₆-C₁₀ aralkyl acyl. In some embodiments, R₈ is substituted C₆-C₁₀ aralkyl acyl. In some embodiments, R₈ is unsubstituted C₆-C₁₀ alkylaryl acyl. In some embodiments, R₈ is substituted C₆-C₁₀ alkylaryl acyl. In some embodiments, R₈ is unsubstituted alkoxy. In some embodiments, R₈ is substituted alkoxy. In some embodiments, R₈ is unsubstituted aryl. In some embodiments, R₈ is substituted aryl. In some embodiments, R₈ is unsubstituted C₃-C₁₀heterocyclyl. In some embodiments, R₈ is substituted C₃-C₁₀ heterocyclyl. In some embodiments, R₈ is unsubstituted heteroaryl, In some embodiments, R₈ is unsubstituted C₃-C₁₀cycloalkyl. In some embodiments, R₈ is substituted C₃-C₁₀cycloalkyl. In some embodiments, R₈ is —OPO₃WY. In some embodiments, R₈ is —OCH₂PO₄WY. In some embodiments, R₈ is —OCH₂PO₄Z. In some embodiments, R₈ is —OPO₃Z.

In some embodiments, R₉ is hydrogen. In some embodiments, R₉ is hydroxyl. In some embodiments, R₉ is carboxaldehyde. In some embodiments, R₉ is unsubstituted amine. In some embodiments, R₉ is substituted amine. In some embodiments, R₉ is unsubstituted C₁-C₁₀ alkyl. In some embodiments, R₉ is substituted C₁-C₁₀ alkyl. In some embodiments, R₉ is unsubstituted C₂-C₁₀ alkynyl. In some embodiments, R₉ is substituted C₂-C₁₀ alkynyl. In some embodiments, R₉ is unsubstituted C₂-C₁₀ alkenyl. In some embodiments, R₉ is substituted C₂-C₁₀ alkenyl. In some embodiments, R₉ is carboxyl. In some embodiments, R₉ is unsubstituted carbohydrate. In some embodiments, R₉ is substituted carbohydrate. In some embodiments, R₉ is unsubstituted ester. In some embodiments, R₉ is substituted ester. In some embodiments, R₉ is unsubstituted acyloxy. In some embodiments, R₉ is substituted acyloxy. In some embodiments, R₉ is nitro. In some embodiments, R₉ is halogen. In some embodiments, R₉ is unsubstituted C₁-C₁₀ aliphatic acyl. In some embodiments, R₉ is substituted C₁-C₁₀ aliphatic acyl. In some embodiments, R₉ is unsubstituted C₆-C₁₁ aromatic acyl. In some embodiments, R₉ is substituted C₆-C₁ aromatic acyl. In some embodiments, R₉ is unsubstituted C₆-C₁₁ aralkyl acyl. In some embodiments, R₉ is substituted C₆-C₁₀ aralkyl acyl. In some embodiments, R₉ is unsubstituted C₆-C₁₀ alkylaryl acyl. In some embodiments, R₉ is substituted C₆-C₁₀ alkylaryl acyl. In some embodiments, R₉ is unsubstituted alkoxy. In some embodiments, R₉ is substituted alkoxy. In some embodiments, R₉ is unsubstituted aryl. In some embodiments, R₉ is substituted aryl. In some embodiments, R₉ is unsubstituted C₃-C₁₀heterocyclyl. In some embodiments, R₉ is substituted C₃-C₁₀ heterocyclyl. In some embodiments, R₉ is unsubstituted heteroaryl, In some embodiments, R₉ is unsubstituted C₃-C₁₀cycloalkyl. In some embodiments, R₉ is substituted C₃-C₁₀cycloalkyl. In some embodiments, R₉ is —OPO₃WY. In some embodiments, R₉ is —OCH₂PO₄WY. In some embodiments, R₉ is —OCH₂PO₄Z. In some embodiments, R₉ is —OPO₃Z.

In some embodiments, R₁₆ is hydrogen. In some embodiments, R₁₆ is unsubstituted C₁-C₁₀ alkyl. In some embodiments, R₁₆ is substituted C₁-C₁₀ alkyl. In some embodiments, R₁₆ is unsubstituted C₂-C₁₀ alkynyl. In some embodiments, R₁₆ is substituted C₂-C₁₀ alkynyl. In some embodiments, R₁₆ is unsubstituted C₂-C₁₀ alkenyl. In some embodiments, R₁₆ is substituted C₂-C₁₀ alkenyl. In some embodiments, R₁₆ is unsubstituted carbohydrate. In some embodiments, R₁₆ is substituted carbohydrate. In some embodiments, R₁₆ is unsubstituted C₁-C₁₀ aliphatic acyl. In some embodiments, R₁₆ is substituted C₁-C₁₀ aliphatic acyl. In some embodiments, R₁₆ is unsubstituted C₆-C₁₀ aromatic acyl. In some embodiments, R₁₆ is substituted C₆-C₁₀ aromatic acyl. In some embodiments, R₁₆ is unsubstituted C₆-C₁₀ aralkyl acyl. In some embodiments, R₁₆ is substituted C₆-C₁₀ aralkyl acyl. In some embodiments, R₁₆ is unsubstituted C₆-C₁₀ alkylaryl acyl. In some embodiments, R₁₆ is substituted C₆-C₁₀ alkylaryl acyl. In some embodiments, R₁₆ is unsubstituted aryl. In some embodiments, R₁₆ is substituted aryl. In some embodiments, R₁₆ is unsubstituted C₃-C₁₀heterocyclyl. In some embodiments, R₁₆ is substituted C₃-C₁₀ heterocyclyl. In some embodiments, R₁₆ is unsubstituted heteroaryl. In some embodiments, R₁₆ is substituted heteroaryl. In some embodiments, R₁₆ is unsubstituted C₃-C₁₀cycloalkyl. In some embodiments, R₁₆ is substituted C₃-C₁₀cycloalkyl. In some embodiments, R₁₆ is —PO₃WY. In some embodiments, R₁₆ is —CH₂PO₄WY. In some embodiments, R₁₆ is —CH₂PO₄Z. In some embodiments, R₁₆ is —PO₃Z.

In some embodiments, R₁₈ is hydrogen. In some embodiments, R₁₈ is hydroxy. In some embodiments, R₁₈ is carboxaldehyde. In some embodiments, R₁₈ is unsubstituted amine. In some embodiments, R₁₈ is substituted amine. In some embodiments, R₁₈ is unsubstituted C₁-C₁₀ alkyl. In some embodiments, R₁₈ is unsubstituted C₂-C₁₀ alkynyl. In some embodiments, R₁₈ is substituted C₂-C₁₀ alkynyl. In some embodiments, R₁₈ is unsubstituted C₂-C₁₀ alkenyl. In some embodiments, R₁₈ is substituted C₂-C₁₀ alkenyl. In some embodiments, R₁₈ is carboxyl. In some embodiments, R₁₈ is unsubstituted carbohydrate. In some embodiments, R₁₈ is substituted carbohydrate. In some embodiments, R₁₈ is substituted carbohydrate. In some embodiments, R₁₈ is unsubstituted ester. In some embodiments, R₁₈ is substituted ester. In some embodiments, R₁₈ is unsubstituted acyloxy. In some embodiments, R₁₈ is substituted acyloxy. In some embodiments, R₁₈ is nitro. In some embodiments, R₁₈ is halogen. In some embodiments, R₁₈ is unsubstituted C₁-C₁₀ aliphatic acyl. In some embodiments, R₁₈ is substituted C₁-C₁₀ aliphatic acyl. In some embodiments, R₁₈ is unsubstituted C₆-C₁₀ aromatic acyl. In some embodiments, R₁₈ is substituted C₆-C₁₀ aromatic acyl. In some embodiments, R₁₈ is unsubstituted C₆-C₁₀ aralkyl acyl. In some embodiments, R₁₈ is substituted C₆-C₁₀ aralkyl acyl. In some embodiments, R₁₈ is unsubstituted C₆-C₁₀ alkylaryl acyl. In some embodiments, R₁₈ is substituted C₆-C₁₀ alkylaryl acyl. In some embodiments, R₁₈ is unsubstituted alkoxy. In some embodiments, R₁₈ is substituted alkoxy. In some embodiments, R₁₈ is unsubstituted aryl. In some embodiments, R₁₈ is substituted aryl. In some embodiments, R₁₈ is unsubstituted C₃-C₁₀heterocyclyl. In some embodiments, R₁₈ is substituted C₃-C₁₀heterocyclyl. In some embodiments, R₁₈ is unsubstituted heteroaryl. In some embodiments, R₁₈ is substituted heteroaryl. In some embodiments, R₁₈ is unsubstituted C₃-C₁₀cycloalkyl. In some embodiments, R₁₈ is substituted C₃-C₁₀cycloalkyl. In some embodiments, R₁₈ is —OPO₃WY. In some embodiments, R₁₈ is —OCH₂PO₄WY. In some embodiments, R₁₈ is —OCH₂PO₄Z. In some embodiments, R₁₈ is —OPO₃Z.

In some embodiments, R₁₉ is hydrogen. In some embodiments, R₁₉ is unsubstituted C₁-C₁₀ alkyl. In some embodiments, R₁₉ is substituted C₁-C₁₀ alkyl. In some embodiments, R₁₉ is unsubstituted C₂-C₁₀ alkynyl. In some embodiments, R₁₉ is substituted C₂-C₁₀ alkynyl. In some embodiments, R₁₉ is unsubstituted C₂-C₁₀ alkenyl. In some embodiments, R₁₉ is substituted C₂-C₁₀ alkenyl. In some embodiments, R₁₉ is unsubstituted carbohydrate. In some embodiments, R₁₉ is substituted carbohydrate. In some embodiments, R₁₉ is unsubstituted C₁-C₁₀ aliphatic acyl. In some embodiments, R₁₉ is substituted C₁-C₁₀ aliphatic acyl. In some embodiments, R₁₉ is unsubstituted C₆-C₁₀ aromatic acyl. In some embodiments, R₁₉ is substituted C₆-C₁₀ aromatic acyl. In some embodiments, R₁₉ is unsubstituted C₆-C₁₀ aralkyl acyl. In some embodiments, R₁₉ is substituted C₆-C₁₀ aralkyl acyl. In some embodiments, R₁₉ is unsubstituted C₆-C₁₀ alkylaryl acyl. In some embodiments, R₁₉ is substituted C₆-C₁₀ alkylaryl acyl. In some embodiments, R₁₉ is unsubstituted aryl. In some embodiments, R₁₉ is substituted aryl. In some embodiments, R₁₉ is unsubstituted C₃-C₁₀heterocyclyl. In some embodiments, R₁₉ is substituted C₃-C₁₀ heterocyclyl. In some embodiments, R₁₉ is unsubstituted heteroaryl. In some embodiments, R₁₉ is substituted heteroaryl. In some embodiments, R₁₉ is unsubstituted C₃-C₁₀cycloalkyl. In some embodiments, R₁₉ is substituted C₃-C₁₀cycloalkyl. In some embodiments, R₁₉ is —PO₃WY. In some embodiments, R₁₉ is —CH₂PO₄WY. In some embodiments, R₁₉ is —CH₂PO₄Z. In some embodiments, R₁₉ is —PO₃Z.

In some embodiments, s is an integer of 0. In some embodiments, s is an integer of 1. In some embodiments, s is an integer of 2. In some embodiments, s is an integer of 3.

In various embodiments, W is hydrogen. In various embodiments, W is unsubstituted methyl. In various embodiments, W is substituted methyl. In various embodiments, W is unsubstituted ethyl. In various embodiments, W is substituted ethyl. In various embodiments, W is unsubstituted alkyl. In various embodiments, W is substituted alkyl. In various embodiments, W is unsubstituted carbohydrate. In various embodiments, W is substituted carbohydrate. In various embodiments, W is potassium. In various embodiments, W is sodium. In various embodiments, W is lithium.

In various embodiments, Y is hydrogen. In various embodiments, Y is unsubstituted methyl. In various embodiments, Y is substituted methyl. In various embodiments, Y is unsubstituted ethyl. In various embodiments, Y is substituted ethyl. In various embodiments, Y is unsubstituted alkyl. In various embodiments, Y is substituted alkyl. In various embodiments, Y is unsubstituted carbohydrate. In various embodiments, Y is substituted carbohydrate. In various embodiments, Y is potassium. In various embodiments, Y is sodium. In various embodiments, Y is lithium.

In various embodiments, Z is calcium. In various embodiments, Z is magnesium. In various embodiments, Z is iron.

In other embodiments of the invention, the pyrone analog is of Formula XIII or a pharmaceutically/veterinarily acceptable salt thereof:

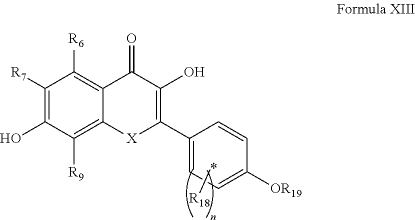

wherein X is O, S, or NR′ wherein R′ is hydrogen, C₁-C₁₀ alkyl, C₂-C₁₀ alkynyl, C₂-C₁₀ alkenyl, C₁-C₁₀ aliphatic acyl, C₆-C₁₀ aromatic acyl, C₆-C₁₀ aralkyl acyl, C₆-C₁₀ alkylaryl acyl, aryl, C₃-C₁₀ heterocyclyl, heteroaryl, or C₃-C₁₀cycloalkyl;

wherein R₂ is hydrogen, hydroxyl, C₁-C₁₀ alkyl, C₂-C₁₀ alkynyl, C₂-C₁₀ alkenyl, carboxyl, carbohydrate, ester, acyloxy, nitro, halogen, C₁-C₁₀ aliphatic acyl, C₆-C₁₀ aromatic acyl, C₆-C₁₀ aralkyl acyl, C₆-C₁₀ alkylaryl acyl, alkoxy, amine, aryl, C₄-C₁₀heterocyclyl, heteroaryl, C₃-C₁₀cycloalkyl, —OPO₃WY, —OCH₂PO₄WY, —OCH₂PO₄Z or —OPO₃Z;

R₆, R₇, and R₉ are independently hydrogen, hydroxyl, carboxaldehyde, amino, C₁-C₁₀ alkyl, C₂-C₁₀ alkynyl, C₂-C₁₀ alkenyl, carboxyl, carbohydrate, ester, acyloxy, nitro, halogen, C₁-C₁₀ aliphatic acyl, C₆-C₁₀ aromatic acyl, C₆-C₁₀ aralkyl acyl, C₆-C₁₀ alkylaryl acyl, alkoxy, amine, aryl, C₃-C₁₀ heterocyclyl, heteroaryl, C₃-C₁₀cycloalkyl, —OPO₃WY, —OCH₂PO₄WY, —OCH₂PO₄Z or —OPO₃Z;

each instance of R₁₈ is independently hydrogen, hydroxyl, carboxaldehyde, amine, C₁-C₁₀ alkyl, C₂-C₁₀ alkynyl, C₂-C₁₀ alkenyl, carboxyl, carbohydrate, ester, acyloxy, nitro, halogen, C₁-C₁₀ aliphatic acyl, C₆-C₁₀ aromatic acyl, C₆-C₁₀ aralkyl acyl, C₆-C₁₀alkylaryl acyl, alkoxy, alkyl, phosphate, aryl, heteroaryl, C₃-C₁₀ heterocyclic, C₃-C₁₀cycloalkyl, —OPO₃WY, —OCH₂PO₄WY, —OCH₂PO₄Z or —OPO₃Z;

R₁₉ is hydrogen, C₁-C₁₀ alkyl, C₂-C₁₀ alkynyl, C₂-C₁₀ alkenyl, carbohydrate, C₁-C₁₀ aliphatic acyl, C₆-C₁₀ aromatic acyl, C₆-C₁₀ aralkyl acyl, C₆-C₁₀ alkylaryl acyl, aryl, C₃-C₁₀heterocyclyl, heteroaryl, optionally substituted C₃-C₁₀cycloalkyl, —PO₃WY, —CH₂PO₄WY, —CH₂PO₄Z or —PO₃Z;

n is an integer of 0, 1, 2, 3, or 4; and

W and Y are independently hydrogen, methyl, ethyl, alkyl, carbohydrate, or a cation, and Z is a multivalent cation; and

wherein the compound of Formula XIII is not the compound of Formula A:

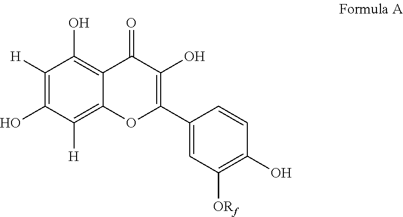

wherein R_{f} is hydrogen; and

wherein the compound of Formula XIII is not the compound wherein:

R₂ is hydroxy, R₆, R₇, and R₉ are hydrogen, n is 2, and a first R₁₈ is 3′-hydroxy, R₁₉ is hydrogen, and a second R₁₈ is 5′-hydroxy; or

R₂ is hydroxy, R₆, R₇, and R₉ are hydrogen, n is 1, R₁₉ is hydrogen, and R₁₈ is 3′-hydroxy.

In some embodiments, the compound of Formula XIII is not the compound of Formula A wherein R_{f} is PO₃K₂.

In some embodiments, R₂ is hydrogen. In some embodiments, R₂ is hydroxyl. In some embodiments, R₂ is optionally substituted C₁-C₁₀ alkyl. In some embodiments, R₂ is unsubstituted C₁-C₁₀ alkyl. In some embodiments, R₂ is substituted C₁-C₁₀ alkyl. In some embodiments, R₂ is unsubstituted C₁-C₁₀ alkyl. In some other embodiments, R₂ is substituted C₁-C₁₀ alkyl. In some embodiments, R₂ is unsubstituted C₂-C₁₀ alkynyl. In some embodiments, R₂ is substituted C₂-C₁₀ alkynyl. In some embodiments, R₂ is unsubstituted C₂-C₁₀ alkenyl. In some embodiments, R₂ is substituted C₂-C₁₀ alkenyl. In some embodiments, R₂ is carboxyl. In some embodiments, R₂ is unsubstituted carbohydrate. In some embodiments, R₂ is substituted carbohydrate. In some embodiments, R₂ is unsubstituted ester. In some embodiments, R₂ is substituted ester. In some embodiments, R₂ is unsubstituted acyloxy. In some embodiments, R₂ is substituted acyloxy. In some embodiments, R₂ is nitro. In some embodiments, R₂ is halogen. In some embodiments, R₂ is unsubstituted C₁-C₁₀ aliphatic acyl. In some embodiments, R₂ is substituted C₁-C₁₀ aliphatic acyl. In some embodiments, R₂ is unsubstituted C₆-C₁₀ aromatic acyl. In some embodiments, R₂ is substituted C₆-C₁₀ aromatic acyl. In some embodiments, R₂ is unsubstituted C₆-C₁₀ aralkyl acyl. In some embodiments, R₂ is substituted C₆-C₁₀ aralkyl acyl. In some embodiments, R₂ is unsubstituted C₆-C₁₀ alkylaryl acyl. In some embodiments, R₂ is substituted C₆-C₁₀ alkylaryl acyl. In some embodiments, R₂ is unsubstituted alkoxy. In some embodiments, R₂ is substituted alkoxy. In some embodiments, R₂ is unsubstituted amine. In some embodiments, R₂ is substituted amine. In some embodiments, R₂ is unsubstituted aryl. In some embodiments, R₂ is substituted aryl. In some embodiments, R₂ is unsubstituted C₄-C₁₀heterocyclyl. In some embodiments, R₂ is substituted C₄-C₁₀heterocyclyl. In some embodiments, R₂ is unsubstituted heteroaryl. In some embodiments, R₂ is substituted heteroaryl. In some embodiments, R₂ is unsubstituted C₃-C₁₀cycloalkyl. In some embodiments, R₂ is substituted C₃-C₁₀cycloalkyl. In some embodiments, R₂ is —OPO₃WY. In some embodiments, R₂ is —OCH₂PO₄WY. In some embodiments, R₂ is —OCH₂PO₄Z. In some embodiments, R₂ is —OPO₃Z.

In some embodiments, R₆ is hydrogen. In some embodiments, R₆ is hydroxyl. In some embodiments, R₆ is carboxaldehyde. In some embodiments, R₆ is unsubstituted amine. In some embodiments, R₆ is substituted amine. In some embodiments, R₆ is unsubstituted C₁-C₁₀ alkyl. In some embodiments, R₆ is substituted C₁-C₁₀ alkyl. In some embodiments, R₆ is unsubstituted C₂-C₁₀ alkynyl. In some embodiments, R₆ is substituted C₂-C₁₀ alkynyl. In some embodiments, R₆ is unsubstituted C₂-C₁₀ alkenyl. In some embodiments, R₆ is substituted C₂-C₁₀ alkenyl. In some embodiments, R₆ is carboxyl. In some embodiments, R₆ is unsubstituted carbohydrate. In some embodiments, R₆ is substituted carbohydrate. In some embodiments, R₆ is unsubstituted ester. In some embodiments, R₆ is substituted ester. In some embodiments, R₆ is unsubstituted acyloxy. In some embodiments, R₆ is substituted acyloxy. In some embodiments, R₆ is nitro. In some embodiments, R₆ is halogen. In some embodiments, R₆ is unsubstituted C₁-C₁₀ aliphatic acyl. In some embodiments, R₆ is substituted C₁-C₁₀ aliphatic acyl. In some embodiments, R₆ is unsubstituted C₆-C₁₀ aromatic acyl. In some embodiments, R₆ is substituted C₆-C₁₀ aromatic acyl. In some embodiments, R₆ is unsubstituted C₆-C₁₀ aralkyl acyl. In some embodiments, R₆ is substituted C₆-C₁₀ aralkyl acyl. In some embodiments, R₆ is unsubstituted C₆-C₁₀ alkylaryl acyl. In some embodiments, R₆ is substituted C₆-C₁₀ alkylaryl acyl. In some embodiments, R₆ is unsubstituted alkoxy. In some embodiments, R₆ is substituted alkoxy. In some embodiments, R₆ is unsubstituted aryl. In some embodiments, R₆ is substituted aryl. In some embodiments, R₆ is unsubstituted C₃-C₁₀heterocyclyl. In some embodiments, R₆ is substituted C₃-C₁₀ heterocyclyl. In some embodiments, R₆ is unsubstituted heteroaryl, In some embodiments, R₆ is unsubstituted C₃-C₁₀cycloalkyl. In some embodiments, R₆ is substituted C₃-C₁₀cycloalkyl. In some embodiments, R₆ is —OPO₃WY. In some embodiments, R₆ is —OCH₂PO₄WY. In some embodiments, R₆ is —OCH₂PO₄Z. In some embodiments, R₆ is —OPO₃Z.

In some embodiments, R₇ is hydrogen. In some embodiments, R₇ is hydroxyl. In some embodiments, R₇ is carboxaldehyde. In some embodiments, R₇ is unsubstituted amine. In some embodiments, R₇ is substituted amine. In some embodiments, R₇ is unsubstituted C₁-C₁₀ alkyl. In some embodiments, R₇ is substituted C₁-C₁₀ alkyl. In some embodiments, R₇ is unsubstituted C₂-C₁₀ alkynyl. In some embodiments, R₇ is substituted C₂-C₁₀ alkynyl. In some embodiments, R₇ is unsubstituted C₂-C₁₀ alkenyl. In some embodiments, R₇ is substituted C₂-C₁₀ alkenyl. In some embodiments, R₇ is carboxyl. In some embodiments, R₇ is unsubstituted carbohydrate. In some embodiments, R₇ is substituted carbohydrate. In some embodiments, R₇ is unsubstituted ester. In some embodiments, R₇ is substituted ester. In some embodiments, R₇ is unsubstituted acyloxy. In some embodiments, R₇ is substituted acyloxy. In some embodiments, R₇ is nitro. In some embodiments, R₇ is halogen. In some embodiments, R₇ is unsubstituted C₁-C₁₀ aliphatic acyl. In some embodiments, R₇ is substituted C₁-C₁₀ aliphatic acyl. In some embodiments, R₇ is unsubstituted C₆-C₁₀ aromatic acyl. In some embodiments, R₇ is substituted C₆-C₁₀ aromatic acyl. In some embodiments, R₇ is unsubstituted C₆-C₁₀ aralkyl acyl. In some embodiments, R₇ is substituted C₆-C₁₀ aralkyl acyl. In some embodiments, R₇ is unsubstituted C₆-C₁₀ alkylaryl acyl. In some embodiments, R₇ is substituted C₆-C₁₀ alkylaryl acyl. In some embodiments, R₇ is unsubstituted alkoxy. In some embodiments, R₇ is substituted alkoxy. In some embodiments, R₇ is unsubstituted aryl. In some embodiments, R₇ is substituted aryl. In some embodiments, R₇ is unsubstituted C₃-C₁₀heterocyclyl. In some embodiments, R₇ is substituted C₃-C₁₀ heterocyclyl. In some embodiments, R₇ is unsubstituted heteroaryl, In some embodiments, R₇ is unsubstituted C₃-C₁₀cycloalkyl. In some embodiments, R₇ is substituted C₃-C₁₀cycloalkyl. In some embodiments, R₇ is —OPO₃WY. In some embodiments, R₇ is —OCH₂PO₄WY. In some embodiments, R₇ is —OCH₂PO₄Z. In some embodiments, R₇ is —OPO₃Z.

In some embodiments, R₉ is hydrogen. In some embodiments, R₉ is hydroxyl. In some embodiments, R₉ is carboxaldehyde. In some embodiments, R₉ is unsubstituted amine. In some embodiments, R₉ is substituted amine. In some embodiments, R₉ is unsubstituted C₁-C₁₀ alkyl. In some embodiments, R₉ is substituted C₁-C₁₀ alkyl. In some embodiments, R₉ is unsubstituted C₂-C₁₀ alkynyl. In some embodiments, R₉ is substituted C₂-C₁₀ alkynyl. In some embodiments, R₉ is unsubstituted C₂-C₁₀ alkenyl. In some embodiments, R₉ is substituted C₂-C₁₀ alkenyl. In some embodiments, R₉ is carboxyl. In some embodiments, R₉ is unsubstituted carbohydrate. In some embodiments, R₉ is substituted carbohydrate. In some embodiments, R₉ is unsubstituted ester. In some embodiments, R₉ is substituted ester. In some embodiments, R₉ is unsubstituted acyloxy. In some embodiments, R₉ is substituted acyloxy. In some embodiments, R₉ is nitro. In some embodiments, R₉ is halogen. In some embodiments, R₉ is unsubstituted C₁-C₁₀ aliphatic acyl. In some embodiments, R₉ is substituted C₁-C₁₀ aliphatic acyl. In some embodiments, R₉ is unsubstituted C₆-C₁₁ aromatic acyl. In some embodiments, R₉ is substituted C₆-C₁ aromatic acyl. In some embodiments, R₉ is unsubstituted C₆-C₁₁ aralkyl acyl. In some embodiments, R₉ is substituted C₆-C₁₀ aralkyl acyl. In some embodiments, R₉ is unsubstituted C₆-C₁₀ alkylaryl acyl. In some embodiments, R₉ is substituted C₆-C₁₀ alkylaryl acyl. In some embodiments, R₉ is unsubstituted alkoxy. In some embodiments, R₉ is substituted alkoxy. In some embodiments, R₉ is unsubstituted aryl. In some embodiments, R₉ is substituted aryl. In some embodiments, R₉ is unsubstituted C₃-C₁₀heterocyclyl. In some embodiments, R₉ is substituted C₃-C₁₀ heterocyclyl. In some embodiments, R₉ is unsubstituted heteroaryl, In some embodiments, R₉ is unsubstituted C₃-C₁₀cycloalkyl. In some embodiments, R₉ is substituted C₃-C₁₀cycloalkyl. In some embodiments, R₉ is —OPO₃WY. In some embodiments, R₉ is —OCH₂PO₄WY. In some embodiments, R₉ is —OCH₂PO₄Z. In some embodiments, R₉ is —OPO₃Z.

In some embodiments, R₁₈ is hydrogen. In some embodiments, R₁₈ is hydroxy. In some embodiments, R₁₈ is carboxaldehyde. In some embodiments, R₁₈ is unsubstituted amine. In some embodiments, R₁₈ is substituted amine. In some embodiments, R₁₈ is unsubstituted C₁-C₁₀ alkyl. In some embodiments, R₁₈ is unsubstituted C₂-C₁₀ alkynyl. In some embodiments, R₁₈ is substituted C₂-C₁₀ alkynyl. In some embodiments, R₁₈ is unsubstituted C₂-C₁₀ alkenyl. In some embodiments, R₁₈ is substituted C₂-C₁₀ alkenyl. In some embodiments, R₁₈ is carboxyl. In some embodiments, R₁₈ is unsubstituted carbohydrate. In some embodiments, R₁₈ is substituted carbohydrate. In some embodiments, R₁₈ is substituted carbohydrate. In some embodiments, R₁₈ is unsubstituted ester. In some embodiments, R₁₈ is substituted ester. In some embodiments, R₁₈ is unsubstituted acyloxy. In some embodiments, R₁₈ is substituted acyloxy. In some embodiments, R₁₈ is nitro. In some embodiments, R₁₈ is halogen. In some embodiments, R₁₈ is unsubstituted C₁-C₁₀ aliphatic acyl. In some embodiments, R₁₈ is substituted C₁-C₁₀ aliphatic acyl. In some embodiments, R₁₈ is unsubstituted C₆-C₁₀ aromatic acyl. In some embodiments, R₁₈ is substituted C₆-C₁₀ aromatic acyl. In some embodiments, R₁₈ is unsubstituted C₆-C₁₀ aralkyl acyl. In some embodiments, R₁₈ is substituted C₆-C₁₀ aralkyl acyl. In some embodiments, R₁₈ is unsubstituted C₆-C₁₀ alkylaryl acyl. In some embodiments, R₁₈ is substituted C₆-C₁₀ alkylaryl acyl. In some embodiments, R₁₈ is unsubstituted alkoxy. In some embodiments, R₁₈ is substituted alkoxy. In some embodiments, R₁₈ is unsubstituted aryl. In some embodiments, R₁₈ is substituted aryl. In some embodiments, R₁₈ is unsubstituted C₃-C₁₀heterocyclyl. In some embodiments, R₁₈ is substituted C₃-C₁₀heterocyclyl. In some embodiments, R₁₈ is unsubstituted heteroaryl. In some embodiments, R₁₈ is substituted heteroaryl. In some embodiments, R₁₈ is unsubstituted C₃-C₁₀cycloalkyl. In some embodiments, R₁₈ is substituted C₃-C₁₀cycloalkyl. In some embodiments, R₁₈ is —OPO₃WY. In some embodiments, R₁₈ is —OCH₂PO₄WY. In some embodiments, R₁₈ is —OCH₂PO₄Z. In some embodiments, R₁₈ is —OPO₃Z.

In some embodiments, R₁₉ is hydrogen. In some embodiments, R₁₉ is unsubstituted C₁-C₁₀ alkyl. In some embodiments, R₁₉ is substituted C₁-C₁₀ alkyl. In some embodiments, R₁₉ is unsubstituted C₂-C₁₀ alkynyl. In some embodiments, R₁₉ is substituted C₂-C₁₀ alkynyl. In some embodiments, R₁₉ is unsubstituted C₂-C₁₀ alkenyl. In some embodiments, R₁₉ is substituted C₂-C₁₀ alkenyl. In some embodiments, R₁₉ is unsubstituted carbohydrate. In some embodiments, R₁₉ is substituted carbohydrate. In some embodiments, R₁₉ is unsubstituted C₁-C₁₀ aliphatic acyl. In some embodiments, R₁₉ is substituted C₁-C₁₀ aliphatic acyl. In some embodiments, R₁₉ is unsubstituted C₆-C₁₀ aromatic acyl. In some embodiments, R₁₉ is substituted C₆-C₁₀ aromatic acyl. In some embodiments, R₁₉ is unsubstituted C₆-C₁₀ aralkyl acyl. In some embodiments, R₁₉ is substituted C₆-C₁₀ aralkyl acyl. In some embodiments, R₁₉ is unsubstituted C₆-C₁₀ alkylaryl acyl. In some embodiments, R₁₉ is substituted C₆-C₁₀ alkylaryl acyl. In some embodiments, R₁₉ is unsubstituted aryl. In some embodiments, R₁₉ is substituted aryl. In some embodiments, R₁₉ is unsubstituted C₃-C₁₀heterocyclyl. In some embodiments, R₁₉ is substituted C₃-C₁₀ heterocyclyl. In some embodiments, R₁₉ is unsubstituted heteroaryl. In some embodiments, R₁₉ is substituted heteroaryl. In some embodiments, R₁₉ is unsubstituted C₃-C₁₀cycloalkyl. In some embodiments, R₁₉ is substituted C₃-C₁₀cycloalkyl. In some embodiments, R₁₉ is —PO₃WY. In some embodiments, R₁₉ is —CH₂PO₄WY. In some embodiments, R₁₉ is —CH₂PO₄Z. In some embodiments, R₁₉ is —PO₃Z.

In some embodiments, n is an integer of 0. In some embodiments, n is an integer of 1. In some embodiments, n is an integer of 2. In some embodiments, n is an integer of 3. In some embodiments, n is an integer of 4.

In various embodiments, W is hydrogen. In various embodiments, W is unsubstituted methyl. In various embodiments, W is substituted methyl. In various embodiments, W is unsubstituted ethyl. In various embodiments, W is substituted ethyl. In various embodiments, W is unsubstituted alkyl. In various embodiments, W is substituted alkyl. In various embodiments, W is unsubstituted carbohydrate. In various embodiments, W is substituted carbohydrate. In various embodiments, W is potassium. In various embodiments, W is sodium. In various embodiments, W is lithium.

In various embodiments, Y is hydrogen. In various embodiments, Y is unsubstituted methyl. In various embodiments, Y is substituted methyl. In various embodiments, Y is unsubstituted ethyl. In various embodiments, Y is substituted ethyl. In various embodiments, Y is unsubstituted alkyl. In various embodiments, Y is substituted alkyl. In various embodiments, Y is unsubstituted carbohydrate. In various embodiments, Y is substituted carbohydrate. In various embodiments, Y is potassium. In various embodiments, Y is sodium. In various embodiments, Y is lithium.

In various embodiments, Z is calcium. In various embodiments, Z is magnesium. In various embodiments, Z is iron.

In some embodiments of the invention, compounds of the following Formulae VIII-A, VIII-B, and VIII-C, are disclosed, where each instance of R_{c}, and R_{d} is independently hydrogen, —OPO₃WY, —OPO₃Z, —OCH₂OPOWY, or —OCH₂OPO₃Z, where W and Y are hydrogen, methyl, ethyl, alkyl, carbohydrate, lithium, sodium or potassium and Z is calcium, magnesium or iron.

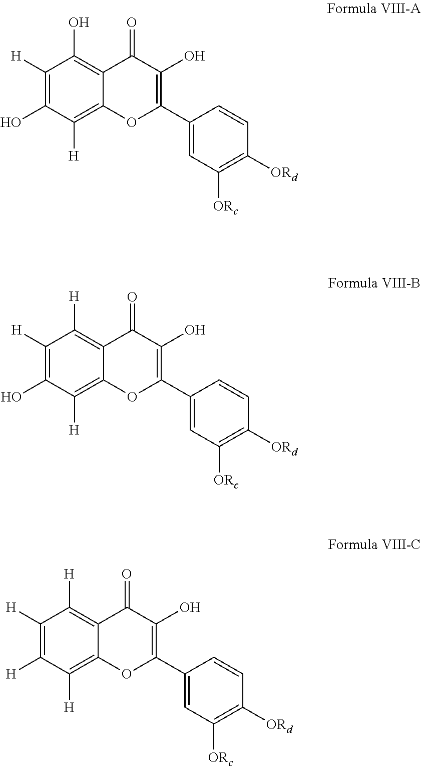

In some embodiments of the invention, for a compound of Formulae VIII-A, VIII-B, or VIII-C, R₁ is OPO₃WY and R_{d} is hydrogen. In some embodiments, the compound of Formula VIII-A is not the compound wherein R₁ is OPO₃K₂ and R_{d} is hydrogen.

In some embodiments of the invention, for a compound of Formulae VIII-A, VIII-B, or VIII-C, R₁ is —OPO₃WY and R_{d} is OPO₃WY. In some embodiments of the invention, for a compound of Formulae VIII-A, VIII-B, or VIII-C, R₁ is a mixture of hydrogen and OPO₃WY and R_{d} is OPO₃WY. In some embodiments of the invention, for a compound of Formulae VIII-A, VIII-B, or VIII-C, R₁ is hydrogen and R_{d} is a mixture of hydrogen and —PO₃Z. In some embodiments of the invention, for a compound of Formulae VIII-A, VIII-B, or VIII-C, R₁ is —OPO₃Z and R_{d} is hydrogen. In some embodiments of the invention, for a compound of Formulae VIII-A, VIII-B, or VIII-C, R_{d} is OPO₃Z and R_{d} is OPO₃Z. In some embodiments of the invention, for a compound of Formulae VIII-A, VIII-B, or VIII-C, R_{d} is a mixture of hydrogen and —OPO₃Z and R_{d} is OPO₃Z. In some embodiments of the invention, for a compound of Formulae VIII-A, VIII-B, or VIII-C, R₁ is hydrogen and R_{d} is a mixture of hydrogen and —OPO₃Z. In some embodiments of the invention, for a compound of Formulae VIII-A, VIII-B, or VIII-C, R_{d} is —CH₂OPO₃Z and R_{d} is hydrogen. In some embodiments of the invention, for a compound of Formulae VIII-A, VIII-B, or VIII-C, R_{d} is —CH₂OPO₃Z and R_{d} is —CH₂OPO₃Z. In some embodiments of the invention, for a compound of Formulae VIII-A, VIII-B, or VIII-C, R₁ is a mixture of hydrogen and —CH₂OPO₃Z and R_{d} is —CH₂OPO₃Z. In some embodiments of the invention, for a compound of Formulae VIII-A, VIII-B, or VIII-C, R₁ is hydrogen and R_{d} is a mixture of hydrogen and —CH₂OPO₃Z.

In some embodiments, the pyrone analog of Formula XIII is of Formula XIV:

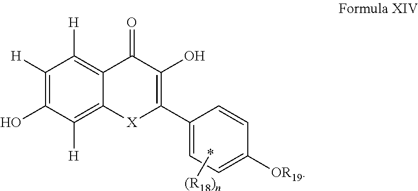

In some embodiments the pyrone analog of the invention is of Formula XV or a pharmaceutically/veterinarily acceptable salt thereof:

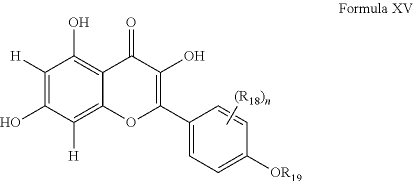

wherein each instance of R₁₈ is independently hydrogen, hydroxyl, carboxaldehyde, amine, C₁-C₁₀ alkyl, C₂-C₁₀ alkynyl, C₂-C₁₀ alkenyl, carboxyl, carbohydrate, ester, acyloxy, nitro, halogen, C₁-C₁₀ aliphatic acyl, C₆-C₁₀ aromatic acyl, C₆-C₁₀ aralkyl acyl, C₆-C₁₀ alkylaryl acyl, alkoxy, alkyl, phosphate, aryl, heteroaryl, C₃-C₁₀ heterocyclic, C₃-C₁₀cycloalkyl, —OPO₃WY, —OCH₂PO₄WY, —OCH₂PO₄Z or —OPO₃Z;

R₁₉ is hydrogen, C₁-C₁₀ alkyl, C₂-C₁₀ alkynyl, C₂-C₁₀ alkenyl, carbohydrate, C₁-C₁₀ aliphatic acyl, C₆-C₁₀ aromatic acyl, C₆-C₁₀ aralkyl acyl, C₆-C₁₀ alkylaryl acyl, aryl, C₃-C₁₀heterocyclyl, heteroaryl, optionally substituted C₃-C₁₀cycloalkyl, —PO₃WY, —CH₂PO₄WY, —CH₂PO₄Z or —PO₃Z;

n is an integer of 0, 1, 2, 3, or 4; and

W and Y are independently hydrogen, methyl, ethyl, alkyl, carbohydrate, or a cation, and Z is a multivalent cation; and

wherein the compound of Formula XV is not the compound of Formula A:

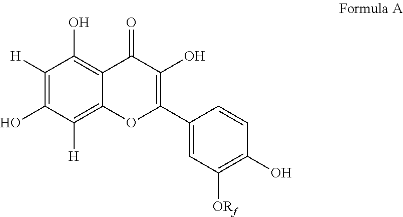

wherein R_{f} is hydrogen.

In some embodiments, the compound of Formula XV is not the compound of Formula A wherein R_{f} is PO₃K₂.

In some embodiments of the invention, R₁₉ is —PO₃WY, —CH₂PO₄WY, —CH₂PO₄Z or —PO₃Z, or at least one of R₁₈ is —OPO₃WY, —OCH₂PO₄WY, —OCH₂PO₄Z or —OPO₃Z. In some embodiments, R₁₉ is —PO₃WY, —CH₂PO₄WY, —CH₂PO₄Z or —PO₃Z and at least one of R₁₈ is —OPO₃WY, —OCH₂PO₄WY, —OCH₂PO₄Z or —OPO₃Z.

In some embodiments, R₁₈ is hydrogen. In some embodiments, R₁₈ is hydroxy. In some embodiments, R₁₈ is carboxaldehyde. In some embodiments, R₁₈ is unsubstituted amine. In some embodiments, R₁₈ is substituted amine. In some embodiments, R₁₈ is unsubstituted C₁-C₁₀ alkyl. In some embodiments, R₁₈ is unsubstituted C₂-C₁₀ alkynyl. In some embodiments, R₁₈ is substituted C₂-C₁₀ alkynyl. In some embodiments, R₁₈ is unsubstituted C₂-C₁₀ alkenyl. In some embodiments, R₁₈ is substituted C₂-C₁₀ alkenyl. In some embodiments, R₁₈ is carboxyl. In some embodiments, R₁₈ is unsubstituted carbohydrate. In some embodiments, R₁₈ is substituted carbohydrate. In some embodiments, R₁₈ is substituted carbohydrate. In some embodiments, R₁₈ is unsubstituted ester. In some embodiments, R₁₈ is substituted ester. In some embodiments, R₁₈ is unsubstituted acyloxy. In some embodiments, R₁₈ is substituted acyloxy. In some embodiments, R₁₈ is nitro. In some embodiments, R₁₈ is halogen. In some embodiments, R₁₈ is unsubstituted C₁-C₁₀ aliphatic acyl. In some embodiments, R₁₈ is substituted C₁-C₁₀ aliphatic acyl. In some embodiments, R₁₈ is unsubstituted C₆-C₁₀ aromatic acyl. In some embodiments, R₁₈ is substituted C₆-C₁₀ aromatic acyl. In some embodiments, R₁₈ is unsubstituted C₆-C₁₀ aralkyl acyl. In some embodiments, R₁₈ is substituted C₆-C₁₀ aralkyl acyl. In some embodiments, R₁₈ is unsubstituted C₆-C₁₀ alkylaryl acyl. In some embodiments, R₁₈ is substituted C₆-C₁₀ alkylaryl acyl. In some embodiments, R₁₈ is unsubstituted alkoxy. In some embodiments, R₁₈ is substituted alkoxy. In some embodiments, R₁₈ is unsubstituted aryl. In some embodiments, R₁₈ is substituted aryl. In some embodiments, R₁₈ is unsubstituted C₃-C₁₀heterocyclyl. In some embodiments, R₁₈ is substituted C₃-C₁₀heterocyclyl. In some embodiments, R₁₈ is unsubstituted heteroaryl. In some embodiments, R₁₈ is substituted heteroaryl. In some embodiments, R₁₈ is unsubstituted C₃-C₁₀cycloalkyl. In some embodiments, R₁₈ is substituted C₃-C₁₀cycloalkyl. In some embodiments, R₁₈ is —OPO₃WY. In some embodiments, R₁₈ is —OCH₂PO₄WY. In some embodiments, R₁₈ is —OCH₂PO₄Z. In some embodiments, R₁₈ is —OPO₃Z.

In some embodiments, R₁₉ is hydrogen. In some embodiments, R₁₉ is unsubstituted C₁-C₁₀ alkyl. In some embodiments, R₁₉ is substituted C₁-C₁₀ alkyl. In some embodiments, R₁₉ is unsubstituted C₂-C₁₀ alkynyl. In some embodiments, R₁₉ is substituted C₂-C₁₀ alkynyl. In some embodiments, R₁₉ is unsubstituted C₂-C₁₀ alkenyl. In some embodiments, R₁₉ is substituted C₂-C₁₀ alkenyl. In some embodiments, R₁₉ is unsubstituted carbohydrate. In some embodiments, R₁₉ is substituted carbohydrate. In some embodiments, R₁₉ is unsubstituted C₁-C₁₀ aliphatic acyl. In some embodiments, R₁₉ is substituted C₁-C₁₀ aliphatic acyl. In some embodiments, R₁₉ is unsubstituted C₆-C₁₀ aromatic acyl. In some embodiments, R₁₉ is substituted C₆-C₁₀ aromatic acyl. In some embodiments, R₁₉ is unsubstituted C₆-C₁₀ aralkyl acyl. In some embodiments, R₁₉ is substituted C₆-C₁₀ aralkyl acyl. In some embodiments, R₁₉ is unsubstituted C₆-C₁₀ alkylaryl acyl. In some embodiments, R₁₉ is substituted C₆-C₁₀ alkylaryl acyl. In some embodiments, R₁₉ is unsubstituted aryl. In some embodiments, R₁₉ is substituted aryl. In some embodiments, R₁₉ is unsubstituted C₃-C₁₀heterocyclyl. In some embodiments, R₁₉ is substituted C₃-C₁₀ heterocyclyl. In some embodiments, R₁₉ is unsubstituted heteroaryl. In some embodiments, R₁₉ is substituted heteroaryl. In some embodiments, R₁₉ is unsubstituted C₃-C₁₀cycloalkyl. In some embodiments, R₁₉ is substituted C₃-C₁₀cycloalkyl. In some embodiments, R₁₉ is —PO₃WY. In some embodiments, R₁₉ is —CH₂PO₄WY. In some embodiments, R₁₉ is —CH₂PO₄Z. In some embodiments, R₁₉ is —PO₃Z.

In some embodiments, n is an integer of 0. In some embodiments, n is an integer of 1. In some embodiments, n is an integer of 2. In some embodiments, n is an integer of 3. In some embodiments, n is an integer of 4.

In various embodiments, W is hydrogen. In various embodiments, W is unsubstituted methyl. In various embodiments, W is substituted methyl. In various embodiments, W is unsubstituted ethyl. In various embodiments, W is substituted ethyl. In various embodiments, W is unsubstituted alkyl. In various embodiments, W is substituted alkyl. In various embodiments, W is unsubstituted carbohydrate. In various embodiments, W is substituted carbohydrate. In various embodiments, W is potassium. In various embodiments, W is sodium. In various embodiments, W is lithium.

In various embodiments, Y is hydrogen. In various embodiments, Y is unsubstituted methyl. In various embodiments, Y is substituted methyl. In various embodiments, Y is unsubstituted ethyl. In various embodiments, Y is substituted ethyl. In various embodiments, Y is unsubstituted alkyl. In various embodiments, Y is substituted alkyl. In various embodiments, Y is unsubstituted carbohydrate. In various embodiments, Y is substituted carbohydrate. In various embodiments, Y is potassium. In various embodiments, Y is sodium. In various embodiments, Y is lithium.

In various embodiments, Z is calcium. In various embodiments, Z is magnesium. In various embodiments, Z is iron.

In some embodiments, the pyrone analog of the invention is a compound of Formula XVI or a pharmaceutically/veterinarily acceptable salt thereof:

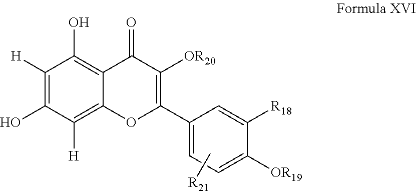

wherein R₁₈ and R₂₁ are independently hydrogen, hydroxyl, carboxaldehyde, amine, C₁-C₁₀ alkyl C₂-C₁₀ alkynyl, C₂-C₁₀ alkenyl, carboxyl, carbohydrate, ester, acyloxy, nitro, halogen, C₁-C₁₀ aliphatic acyl, C₆-C₁₀ aromatic acyl, C₆-C₁₀ aralkyl acyl, C₆-C₁₀alkylaryl acyl, alkoxy, alkyl phosphate, aryl, heteroaryl, C₃-C₁₀ heterocyclic, C₃-C₁₀cycloalkyl, —OPO₃WY, —OCH₂PO₄WY, —OCH₂PO₄Z or —OPO₃Z;

R₁₉ and R₂₀ are independently hydrogen, C₁-C₁₀ alkyl C₂-C₁₀ alkynyl, C₂-C₁₀ alkenyl, carbohydrate, C₁-C₁₀ aliphatic acyl, C₆-C₁₀ aromatic acyl, C₆-C₁₀ aralkyl acyl, C₆-C₁₀ alkylaryl acyl, aryl, C₃-C₁₀ heterocyclyl, heteroaryl, optionally substituted C₃-C₁₀cycloalkyl, —PO₃WY, —CH₂PO₄WY, —CH₂PO₄Z or —PO₃Z; and

W and Y are independently hydrogen, methyl, ethyl, alkyl carbohydrate, or a cation, and Z is a multivalent cation; and

wherein the compound of Formula XVI is not the compound of Formula A:

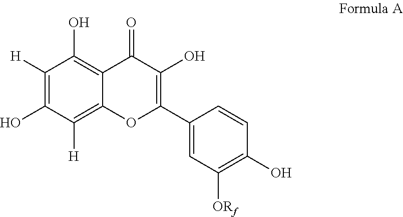

wherein R_{f} is hydrogen.

In some embodiments, the compound of Formula XVI is not the compound of Formula A wherein R_{f} is PO₃K₂.

In some embodiments of the invention, R₁₉ is —PO₃WY, —CH₂PO₄WY, —CH₂PO₄Z or —PO₃Z, or at least one of R₁₈ or R₂₁ is —OPO₃WY, —OCH₂PO₄WY, —OCH₂PO₄Z or —OPO₃Z. In some embodiments, R₁₉ is —PO₃WY, —CH₂PO₄WY, —CH₂PO₄Z or —PO₃Z and at least one of R₁₈ or R₂₁ is —OPO₃WY, —OCH₂PO₄WY, —OCH₂PO₄Z or —OPO₃Z.

In some embodiments, R₁₈ is hydrogen. In some embodiments, R₁₈ is hydroxy. In some embodiments, R₁₈ is carboxaldehyde. In some embodiments, R₁₈ is unsubstituted amine. In some embodiments, R₁₈ is substituted amine. In some embodiments, R₁₈ is unsubstituted C₁-C₁₀ alkyl. In some embodiments, R₁₈ is unsubstituted C₂-C₁₀ alkynyl. In some embodiments, R₁₈ is substituted C₂-C₁₀ alkynyl. In some embodiments, R₁₈ is unsubstituted C₂-C₁₀ alkenyl. In some embodiments, R₁₈ is substituted C₂-C₁₀ alkenyl. In some embodiments, R₁₈ is carboxyl. In some embodiments, R₁₈ is unsubstituted carbohydrate. In some embodiments, R₁₈ is substituted carbohydrate. In some embodiments, R₁₈ is substituted carbohydrate. In some embodiments, R₁₈ is unsubstituted ester. In some embodiments, R₁₈ is substituted ester. In some embodiments, R₁₈ is unsubstituted acyloxy. In some embodiments, R₁₈ is substituted acyloxy. In some embodiments, R₁₈ is nitro. In some embodiments, R₁₈ is halogen. In some embodiments, R₁₈ is unsubstituted C₁-C₁₀ aliphatic acyl. In some embodiments, R₁₈ is substituted C₁-C₁₀ aliphatic acyl. In some embodiments, R₁₈ is unsubstituted C₆-C₁₀ aromatic acyl. In some embodiments, R₁₈ is substituted C₆-C₁₀ aromatic acyl. In some embodiments, R₁₈ is unsubstituted C₆-C₁₀ aralkyl acyl. In some embodiments, R₁₈ is substituted C₆-C₁₀ aralkyl acyl. In some embodiments, R₁₈ is unsubstituted C₆-C₁₀ alkylaryl acyl. In some embodiments, R₁₈ is substituted C₆-C₁₀ alkylaryl acyl. In some embodiments, R₁₈ is unsubstituted alkoxy. In some embodiments, R₁₈ is substituted alkoxy. In some embodiments, R₁₈ is unsubstituted aryl. In some embodiments, R₁₈ is substituted aryl. In some embodiments, R₁₈ is unsubstituted C₃-C₁₀heterocyclyl. In some embodiments, R₁₈ is substituted C₃-C₁₀heterocyclyl. In some embodiments, R₁₈ is unsubstituted heteroaryl. In some embodiments, R₁₈ is substituted heteroaryl. In some embodiments, R₁₈ is unsubstituted C₃-C₁₀cycloalkyl. In some embodiments, R₁₈ is substituted C₃-C₁₀cycloalkyl. In some embodiments, R₁₈ is —OPO₃WY. In some embodiments, R₁₈ is —OCH₂PO₄WY. In some embodiments, R₁₈ is —OCH₂PO₄Z. In some embodiments, R₁₈ is —OPO₃Z.

In some embodiments, R₁₉ is hydrogen. In some embodiments, R₁₉ is unsubstituted C₁-C₁₀ alkyl. In some embodiments, R₁₉ is substituted C₁-C₁₀ alkyl. In some embodiments, R₁₉ is unsubstituted C₂-C₁₀ alkynyl. In some embodiments, R₁₉ is substituted C₂-C₁₀ alkynyl. In some embodiments, R₁₉ is unsubstituted C₂-C₁₀ alkenyl. In some embodiments, R₁₉ is substituted C₂-C₁₀ alkenyl. In some embodiments, R₁₉ is unsubstituted carbohydrate. In some embodiments, R₁₉ is substituted carbohydrate. In some embodiments, R₁₉ is unsubstituted C₁-C₁₀ aliphatic acyl. In some embodiments, R₁₉ is substituted C₁-C₁₀ aliphatic acyl. In some embodiments, R₁₉ is unsubstituted C₆-C₁₀ aromatic acyl. In some embodiments, R₁₉ is substituted C₆-C₁₀ aromatic acyl. In some embodiments, R₁₉ is unsubstituted C₆-C₁₀ aralkyl acyl. In some embodiments, R₁₉ is substituted C₆-C₁₀ aralkyl acyl. In some embodiments, R₁₉ is unsubstituted C₆-C₁₀ alkylaryl acyl. In some embodiments, R₁₉ is substituted C₆-C₁₀ alkylaryl acyl. In some embodiments, R₁₉ is unsubstituted aryl. In some embodiments, R₁₉ is substituted aryl. In some embodiments, R₁₉ is unsubstituted C₃-C₁₀heterocyclyl. In some embodiments, R₁₉ is substituted C₃-C₁₀ heterocyclyl. In some embodiments, R₁₉ is unsubstituted heteroaryl. In some embodiments, R₁₉ is substituted heteroaryl. In some embodiments, R₁₉ is unsubstituted C₃-C₁₀cycloalkyl. In some embodiments, R₁₉ is substituted C₃-C₁₀cycloalkyl. In some embodiments, R₁₉ is —PO₃WY. In some embodiments, R₁₉ is —CH₂PO₄WY. In some embodiments, R₁₉ is —CH₂PO₄Z. In some embodiments, R₁₉ is —PO₃Z.

In some embodiments, R₂₀ is hydrogen. In some embodiments, R₂₀ is unsubstituted C₁-C₁₀ alkyl. In some embodiments, R₂₀ is substituted C₁-C₁₀ alkyl. In some embodiments, R₂₀ is unsubstituted C₂-C₁₀ alkynyl. In some embodiments, R₂₀ is substituted C₂-C₁₀ alkynyl. In some embodiments, R₂₀ is unsubstituted C₂-C₁₀ alkenyl. In some embodiments, R₂₀ is substituted C₂-C₁₀ alkenyl. In some embodiments, R₂₀ is unsubstituted carbohydrate. In some embodiments, R₂₀ is substituted carbohydrate. In some embodiments, R₂₀ is unsubstituted C₁-C₁₀ aliphatic acyl. In some embodiments, R₂₀ is substituted C₁-C₁₀ aliphatic acyl. In some embodiments, R₂₀ is unsubstituted C₆-C₁₀ aromatic acyl. In some embodiments, R₂₀ is substituted C₆-C₁₀ aromatic acyl. In some embodiments, R₂₀ is unsubstituted C₆-C₁₀ aralkyl acyl. In some embodiments, R₂₀ is substituted C₆-C₁₀ aralkyl acyl. In some embodiments, R₂₀ is unsubstituted C₆-C₁₀ alkylaryl acyl. In some embodiments, R₂₀ is substituted C₆-C₁₀ alkylaryl acyl. In some embodiments, R₂₀ is unsubstituted aryl. In some embodiments, R₂₀ is substituted aryl. In some embodiments, R₂₀ is unsubstituted C₃-C₁₀heterocyclyl. In some embodiments, R₂₀ is substituted C₃-C₁₀ heterocyclyl. In some embodiments, R₂₀ is unsubstituted heteroaryl. In some embodiments, R₂₀ is substituted heteroaryl. In some embodiments, R₂₀ is unsubstituted C₃-C₁₀cycloalkyl. In some embodiments, R₂₀ is substituted C₃-C₁₀cycloalkyl. In some embodiments, R₂₀ is —PO₃WY. In some embodiments, R₂₀ is —CH₂PO₄WY. In some embodiments, R₂₀ is —CH₂PO₄Z. In some embodiments, R₂₀ is —PO₃Z.

In some embodiments, R₂₁ is hydrogen. In some embodiments, R₂₁ is hydroxy. In some embodiments, R₂₁ is carboxaldehyde. In some embodiments, R₂₁ is unsubstituted amine. In some embodiments, R₂₁ is substituted amine. In some embodiments, R₂₁ is unsubstituted C₁-C₁₀ alkyl. In some embodiments, R₂₁ is unsubstituted C₂-C₁₀ alkynyl. In some embodiments, R₂₁ is substituted C₂-C₁₀ alkynyl. In some embodiments, R₂₁ is unsubstituted C₂-C₁₀ alkenyl. In some embodiments, R₂₁ is substituted C₂-C₁₀ alkenyl. In some embodiments, R₂₁ is carboxyl. In some embodiments, R₂₁ is unsubstituted carbohydrate. In some embodiments, R₂₁ is substituted carbohydrate. In some embodiments, R₂₁ is unsubstituted ester. In some embodiments, R₂₁ is substituted ester. In some embodiments, R₂₁ is unsubstituted acyloxy. In some embodiments, R₂₁ is substituted acyloxy. In some embodiments, R₂₁ is nitro. In some embodiments, R₂₁ is halogen. In some embodiments, R₂₁ is unsubstituted C₁-C₁₀ aliphatic acyl. In some embodiments, R₂₁ is substituted C₁-C₁₀ aliphatic acyl. In some embodiments, R₂₁ is unsubstituted C₆-C₁₀ aromatic acyl. In some embodiments, R₂₁ is substituted C₆-C₁₀ aromatic acyl. In some embodiments, R₂₁ is unsubstituted C₆-C₁₀ aralkyl acyl. In some embodiments, R₂₁ is substituted C₆-C₁₀ aralkyl acyl. In some embodiments, R₂₁ is unsubstituted C₆-C₁₀ alkylaryl acyl. In some embodiments, R₂₁ is substituted C₆-C₁₀ alkylaryl acyl. In some embodiments, R₂₁ is unsubstituted alkoxy. In some embodiments, R₂₁ is substituted alkoxy. In some embodiments, R₂₁ is unsubstituted aryl. In some embodiments, R₂₁ is substituted aryl. In some embodiments, R₂₁ is unsubstituted C₃-C₁₀heterocyclyl. In some embodiments, R₂₁ is substituted C₃-C₁₀heterocyclyl. In some embodiments, R₂₁ is unsubstituted heteroaryl. In some embodiments, R₂₁ is substituted heteroaryl. In some embodiments, R₂₁ is unsubstituted C₃-C₁₀cycloalkyl. In some embodiments, R₂₁ is substituted C₃-C₁₀cycloalkyl. In some embodiments, R₂₁ is —OPO₃WY. In some embodiments, R₂₁ is —OCH₂PO₄WY. In some embodiments, R₂₁ is —OCH₂PO₄Z. In some embodiments, R₂₁ is —OPO₃Z.

In various embodiments, W is hydrogen. In various embodiments, W is unsubstituted methyl. In various embodiments, W is substituted methyl. In various embodiments, W is unsubstituted ethyl. In various embodiments, W is substituted ethyl. In various embodiments, W is unsubstituted alkyl. In various embodiments, W is substituted alkyl. In various embodiments, W is unsubstituted carbohydrate. In various embodiments, W is substituted carbohydrate. In various embodiments, W is potassium. In various embodiments, W is sodium. In various embodiments, W is lithium.

In various embodiments, Y is hydrogen. In various embodiments, Y is unsubstituted methyl. In various embodiments, Y is substituted methyl. In various embodiments, Y is unsubstituted ethyl. In various embodiments, Y is substituted ethyl. In various embodiments, Y is unsubstituted alkyl. In various embodiments, Y is substituted alkyl. In various embodiments, Y is unsubstituted carbohydrate. In various embodiments, Y is substituted carbohydrate. In various embodiments, Y is potassium. In various embodiments, Y is sodium. In various embodiments, Y is lithium.

In various embodiments, Z is calcium. In various embodiments, Z is magnesium. In various embodiments, Z is iron.

In some embodiments, the pyrone analog of the invention is a compound of Formula XVII or a pharmaceutically/veterinarily acceptable salt thereof:

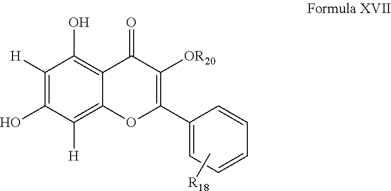

wherein R₁₈ is hydrogen, hydroxyl, carboxaldehyde, amine, C₁-C₁₀ alkyl, C₂-C₁₀ alkynyl, C₂-C₁₀ alkenyl, carboxyl, carbohydrate, ester, acyloxy, nitro, halogen, C₁-C₁₀ aliphatic acyl, C₆-C₁₀ aromatic acyl, C₆-C₁₀ aralkyl acyl, C₆-C₁₀ alkylaryl acyl, alkoxy, alkyl, phosphate, aryl, heteroaryl, C₃-C₁₀ heterocyclic, C₃-C₁₀cycloalkyl, —OPO₃WY, —OCH₂PO₄WY, —OCH₂PO₄Z or —OPO₃Z;

R₂₀ is hydrogen, C₁-C₁₀ alkyl, C₂-C₁₀ alkynyl, C₂-C₁₀ alkenyl, carbohydrate, C₁-C₁₀ aliphatic acyl, C₆-C₁₀ aromatic acyl, C₆-C₁₀ aralkyl acyl, C₆-C₁₀ alkylaryl acyl, aryl, C₃-C₁₀ heterocyclyl, heteroaryl, optionally substituted C₃-C₁₀cycloalkyl, —PO₃WY, —CH₂PO₄WY, —CH₂PO₄Z or —PO₃Z; and

W and Y are independently hydrogen, methyl, ethyl, alkyl, carbohydrate, or a cation, and Z is a multivalent cation.

In some embodiments, R₁₈ is hydrogen. In some embodiments, R₁₈ is hydroxy. In some embodiments, R₁₈ is carboxaldehyde. In some embodiments, R₁₈ is unsubstituted amine. In some embodiments, R₁₈ is substituted amine. In some embodiments, R₁₈ is unsubstituted C₁-C₁₀ alkyl. In some embodiments, R₁₈ is unsubstituted C₂-C₁₀ alkynyl. In some embodiments, R₁₈ is substituted C₂-C₁₀ alkynyl. In some embodiments, R₁₈ is unsubstituted C₂-C₁₀ alkenyl. In some embodiments, R₁₈ is substituted C₂-C₁₀ alkenyl. In some embodiments, R₁₈ is carboxyl. In some embodiments, R₁₈ is unsubstituted carbohydrate. In some embodiments, R₁₈ is substituted carbohydrate. In some embodiments, R₁₈ is substituted carbohydrate. In some embodiments, R₁₈ is unsubstituted ester. In some embodiments, R₁₈ is substituted ester. In some embodiments, R₁₈ is unsubstituted acyloxy. In some embodiments, R₁₈ is substituted acyloxy. In some embodiments, R₁₈ is nitro. In some embodiments, R₁₈ is halogen. In some embodiments, R₁₈ is unsubstituted C₁-C₁₀ aliphatic acyl. In some embodiments, R₁₈ is substituted C₁-C₁₀ aliphatic acyl. In some embodiments, R₁₈ is unsubstituted C₆-C₁₀ aromatic acyl. In some embodiments, R₁₈ is substituted C₆-C₁₀ aromatic acyl. In some embodiments, R₁₈ is unsubstituted C₆-C₁₀ aralkyl acyl. In some embodiments, R₁₈ is substituted C₆-C₁₀ aralkyl acyl. In some embodiments, R₁₈ is unsubstituted C₆-C₁₀ alkylaryl acyl. In some embodiments, R₁₈ is substituted C₆-C₁₀ alkylaryl acyl. In some embodiments, R₁₈ is unsubstituted alkoxy. In some embodiments, R₁₈ is substituted alkoxy. In some embodiments, R₁₈ is unsubstituted aryl. In some embodiments, R₁₈ is substituted aryl. In some embodiments, R₁₈ is unsubstituted C₃-C₁₀heterocyclyl. In some embodiments, R₁₈ is substituted C₃-C₁₀heterocyclyl. In some embodiments, R₁₈ is unsubstituted heteroaryl. In some embodiments, R₁₈ is substituted heteroaryl. In some embodiments, R₁₈ is unsubstituted C₃-C₁₀cycloalkyl. In some embodiments, R₁₈ is substituted C₃-C₁₀cycloalkyl. In some embodiments, R₁₈ is —OPO₃WY. In some embodiments, R₁₈ is —OCH₂PO₄WY. In some embodiments, R₁₈ is —OCH₂PO₄Z. In some embodiments, R₁₈ is —OPO₃Z.

In some embodiments, R₂₀ is hydrogen. In some embodiments, R₂₀ is unsubstituted C₁-C₁₀ alkyl. In some embodiments, R₂₀ is substituted C₁-C₁₀ alkyl. In some embodiments, R₂₀ is unsubstituted C₂-C₁₀ alkynyl. In some embodiments, R₂₀ is substituted C₂-C₁₀ alkynyl. In some embodiments, R₂₀ is unsubstituted C₂-C₁₀ alkenyl. In some embodiments, R₂₀ is substituted C₂-C₁₀ alkenyl. In some embodiments, R₂₀ is unsubstituted carbohydrate. In some embodiments, R₂₀ is substituted carbohydrate. In some embodiments, R₂₀ is unsubstituted C₁-C₁₀ aliphatic acyl. In some embodiments, R₂₀ is substituted C₁-C₁₀ aliphatic acyl. In some embodiments, R₂₀ is unsubstituted C₆-C₁₀ aromatic acyl. In some embodiments, R₂₀ is substituted C₆-C₁₀ aromatic acyl. In some embodiments, R₂₀ is unsubstituted C₆-C₁₀ aralkyl acyl. In some embodiments, R₂₀ is substituted C₆-C₁₀ aralkyl acyl. In some embodiments, R₂₀ is unsubstituted C₆-C₁₀ alkylaryl acyl. In some embodiments, R₂₀ is substituted C₆-C₁₀ alkylaryl acyl. In some embodiments, R₂₀ is unsubstituted aryl. In some embodiments, R₂₀ is substituted aryl. In some embodiments, R₂₀ is unsubstituted C₃-C₁₀heterocyclyl. In some embodiments, R₂₀ is substituted C₃-C₁₀ heterocyclyl. In some embodiments, R₂₀ is unsubstituted heteroaryl. In some embodiments, R₂₀ is substituted heteroaryl. In some embodiments, R₂₀ is unsubstituted C₃-C₁₀cycloalkyl. In some embodiments, R₂₀ is substituted C₃-C₁₀cycloalkyl. In some embodiments, R₂₀ is —PO₃WY. In some embodiments, R₂₀ is —CH₂PO₄WY. In some embodiments, R₂₀ is —CH₂PO₄Z. In some embodiments, R₂₀ is —PO₃Z.

In various embodiments, W is hydrogen. In various embodiments, W is unsubstituted methyl. In various embodiments, W is substituted methyl. In various embodiments, W is unsubstituted ethyl. In various embodiments, W is substituted ethyl. In various embodiments, W is unsubstituted alkyl. In various embodiments, W is substituted alkyl. In various embodiments, W is unsubstituted carbohydrate. In various embodiments, W is substituted carbohydrate. In various embodiments, W is potassium. In various embodiments, W is sodium. In various embodiments, W is lithium.

In various embodiments, Y is hydrogen. In various embodiments, Y is unsubstituted methyl. In various embodiments, Y is substituted methyl. In various embodiments, Y is unsubstituted ethyl. In various embodiments, Y is substituted ethyl. In various embodiments, Y is unsubstituted alkyl. In various embodiments, Y is substituted alkyl. In various embodiments, Y is unsubstituted carbohydrate. In various embodiments, Y is substituted carbohydrate. In various embodiments, Y is potassium. In various embodiments, Y is sodium. In various embodiments, Y is lithium.

In various embodiments, Z is calcium. In various embodiments, Z is magnesium. In various embodiments, Z is iron.

In some embodiments, the pyrone analog of the invention is a compound of Formula XVIII or a pharmaceutically/veterinarily acceptable salt thereof:

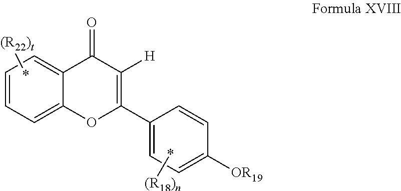

wherein each instance of R₁₈ and R₂₂ is independently hydrogen, hydroxyl, carboxaldehyde, amine, C₁-C₁₀ alkyl, C₂-C₁₀ alkynyl, C₂-C₁₀ alkenyl, carboxyl, carbohydrate, ester, acyloxy, nitro, halogen, C₁-C₁₀ aliphatic acyl, C₆-C₁₀ aromatic acyl, C₆-C₁₀ aralkyl acyl, C₆-C₁₀ alkylaryl acyl, alkoxy, alkyl, phosphate, aryl, heteroaryl, C₃-C₁₀ heterocyclic, C₃-C₁₀cycloalkyl, —OPO₃WY, —OCH₂PO₄WY, —OCH₂PO₄Z or —OPO₃Z;

R₁₉ is hydrogen, C₁-C₁₀ alkyl, C₂-C₁₀ alkynyl, C₂-C₁₀ alkenyl, carbohydrate, C₁-C₁₀ aliphatic acyl, C₆-C₁₀ aromatic acyl, C₆-C₁₀ aralkyl acyl, C₆-C₁₀ alkylaryl acyl, aryl, C₃-C₁₀heterocyclyl, heteroaryl, optionally substituted C₃-C₁₀cycloalkyl, —PO₃WY, —CH₂PO₄WY, —CH₂PO₄Z or —PO₃Z;

n is an integer of 0, 1, 2, 3, or 4;

t is an integer of 0, 1, 2, 3, or 4; and

W and Y are independently hydrogen, methyl, ethyl, alkyl carbohydrate, or a cation, and Z is a multivalent cation; and

wherein the compound of Formula XVIII is not the compound of Formula A:

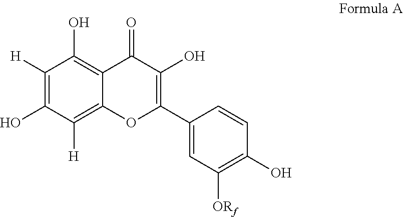

wherein R_{f} is hydrogen; and

wherein the compound of Formula XVIII is not the compound of the following formulae:

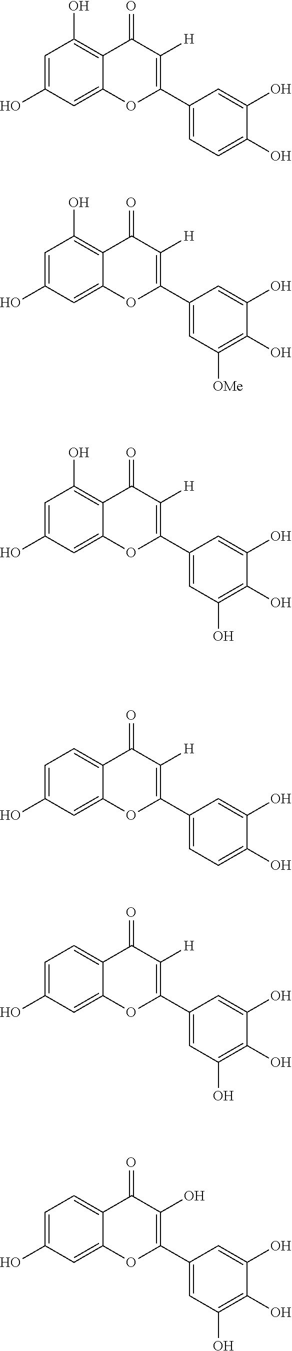

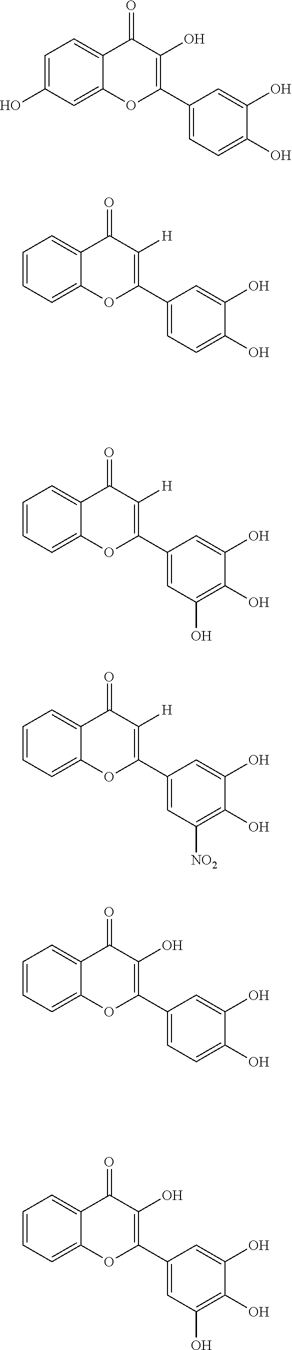

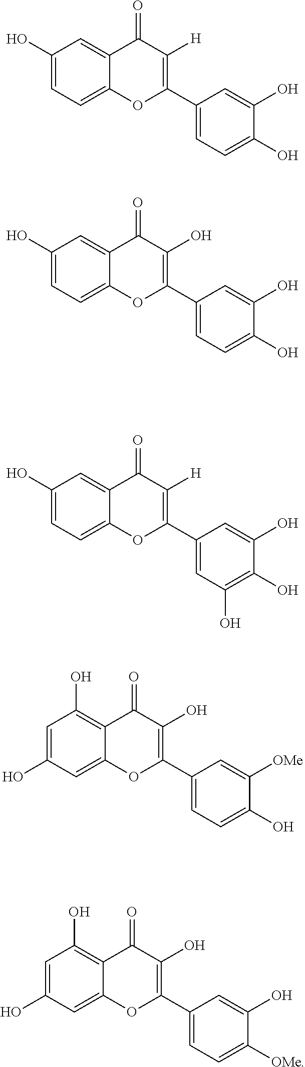

In some embodiments, the compound of Formula XVIII is not the compound of Formula A wherein R_{f} is PO₃K₂.

In some embodiments of the invention, R₁₉ is —PO₃WY, —CH₂PO₄WY, —CH₂PO₄Z or —PO₃Z, or at least one of R₁₈ is —OPO₃WY, —OCH₂PO₄WY, —OCH₂PO₄Z or —OPO₃Z. In some embodiments, R₁₉ is —PO₃WY, —CH₂PO₄WY, —CH₂PO₄Z or —PO₃Z and at least one of R₁₈ is —OPO₃WY, —OCH₂PO₄WY, —OCH₂PO₄Z or —OPO₃Z.

In some embodiments, R₁₈ is hydrogen. In some embodiments, R₁₈ is hydroxy. In some embodiments, R₁₈ is carboxaldehyde. In some embodiments, R₁₈ is unsubstituted amine. In some embodiments, R₁₈ is substituted amine. In some embodiments, R₁₈ is unsubstituted C₁-C₁₀ alkyl. In some embodiments, R₁₈ is unsubstituted C₂-C₁₀ alkynyl. In some embodiments, R₁₈ is substituted C₂-C₁₀ alkynyl. In some embodiments, R₁₈ is unsubstituted C₂-C₁₀ alkenyl. In some embodiments, R₁₈ is substituted C₂-C₁₀ alkenyl. In some embodiments, R₁₈ is carboxyl. In some embodiments, R₁₈ is unsubstituted carbohydrate. In some embodiments, R₁₈ is substituted carbohydrate. In some embodiments, R₁₈ is substituted carbohydrate. In some embodiments, R₁₈ is unsubstituted ester. In some embodiments, R₁₈ is substituted ester. In some embodiments, R₁₈ is unsubstituted acyloxy. In some embodiments, R₁₈ is substituted acyloxy. In some embodiments, R₁₈ is nitro. In some embodiments, R₁₈ is halogen. In some embodiments, R₁₈ is unsubstituted C₁-C₁₀ aliphatic acyl. In some embodiments, R₁₈ is substituted C₁-C₁₀ aliphatic acyl. In some embodiments, R₁₈ is unsubstituted C₆-C₁₀ aromatic acyl. In some embodiments, R₁₈ is substituted C₆-C₁₀ aromatic acyl. In some embodiments, R₁₈ is unsubstituted C₆-C₁₀ aralkyl acyl. In some embodiments, R₁₈ is substituted C₆-C₁₀ aralkyl acyl. In some embodiments, R₁₈ is unsubstituted C₆-C₁₀ alkylaryl acyl. In some embodiments, R₁₈ is substituted C₆-C₁₀ alkylaryl acyl. In some embodiments, R₁₈ is unsubstituted alkoxy. In some embodiments, R₁₈ is substituted alkoxy. In some embodiments, R₁₈ is unsubstituted aryl. In some embodiments, R₁₈ is substituted aryl. In some embodiments, R₁₈ is unsubstituted C₃-C₁₀heterocyclyl. In some embodiments, R₁₈ is substituted C₃-C₁₀heterocyclyl. In some embodiments, R₁₈ is unsubstituted heteroaryl. In some embodiments, R₁₈ is substituted heteroaryl. In some embodiments, R₁₈ is unsubstituted C₃-C₁₀cycloalkyl. In some embodiments, R₁₈ is substituted C₃-C₁₀cycloalkyl. In some embodiments, R₁₈ is —OPO₃WY. In some embodiments, R₁₈ is —OCH₂PO₄WY. In some embodiments, R₁₈ is —OCH₂PO₄Z. In some embodiments, R₁₈ is —OPO₃Z.

In some embodiments, R₁₉ is hydrogen. In some embodiments, R₁₉ is unsubstituted C₁-C₁₀ alkyl. In some embodiments, R₁₉ is substituted C₁-C₁₀ alkyl. In some embodiments, R₁₉ is unsubstituted C₂-C₁₀ alkynyl. In some embodiments, R₁₉ is substituted C₂-C₁₀ alkynyl. In some embodiments, R₁₉ is unsubstituted C₂-C₁₀ alkenyl. In some embodiments, R₁₉ is substituted C₂-C₁₀ alkenyl. In some embodiments, R₁₉ is unsubstituted carbohydrate. In some embodiments, R₁₉ is substituted carbohydrate. In some embodiments, R₁₉ is unsubstituted C₁-C₁₀ aliphatic acyl. In some embodiments, R₁₉ is substituted C₁-C₁₀ aliphatic acyl. In some embodiments, R₁₉ is unsubstituted C₆-C₁₀ aromatic acyl. In some embodiments, R₁₉ is substituted C₆-C₁₀ aromatic acyl. In some embodiments, R₁₉ is unsubstituted C₆-C₁₀ aralkyl acyl. In some embodiments, R₁₉ is substituted C₆-C₁₀ aralkyl acyl. In some embodiments, R₁₉ is unsubstituted C₆-C₁₀ alkylaryl acyl. In some embodiments, R₁₉ is substituted C₆-C₁₀ alkylaryl acyl. In some embodiments, R₁₉ is unsubstituted aryl. In some embodiments, R₁₉ is substituted aryl. In some embodiments, R₁₉ is unsubstituted C₃-C₁₀heterocyclyl. In some embodiments, R₁₉ is substituted C₃-C₁₀ heterocyclyl. In some embodiments, R₁₉ is unsubstituted heteroaryl. In some embodiments, R₁₉ is substituted heteroaryl. In some embodiments, R₁₉ is unsubstituted C₃-C₁₀cycloalkyl. In some embodiments, R₁₉ is substituted C₃-C₁₀cycloalkyl. In some embodiments, R₁₉ is —PO₃WY. In some embodiments, R₁₉ is —CH₂PO₄WY. In some embodiments, R₁₉ is —CH₂PO₄Z. In some embodiments, R₁₉ is —PO₃Z.

In some embodiments, R₂₂ is hydrogen. In some embodiments, R₂₂ is hydroxy. In some embodiments, R₂₂ is carboxaldehyde. In some embodiments, R₂₂ is unsubstituted amine. In some embodiments, R₂₂ is substituted amine. In some embodiments, R₂₂ is unsubstituted C₁-C₁₀ alkyl. In some embodiments, R₂₂ is unsubstituted C₂-C₁₀ alkynyl. In some embodiments, R₂₂ is substituted C₂-C₁₀ alkynyl. In some embodiments, R₂₂ is unsubstituted C₂-C₁₀ alkenyl. In some embodiments, R₂₂ is substituted C₂-C₁₀ alkenyl. In some embodiments, R₂₂ is carboxyl. In some embodiments, R₂₂ is unsubstituted carbohydrate. In some embodiments, R₂₂ is substituted carbohydrate. In some embodiments, R₂₂ is unsubstituted ester. In some embodiments, R₂₂ is substituted ester. In some embodiments, R₂₂ is unsubstituted acyloxy. In some embodiments, R₂₂ is substituted acyloxy. In some embodiments, R₂₂ is nitro. In some embodiments, R₂₂ is halogen. In some embodiments, R₂₂ is unsubstituted C₁-C₁₀ aliphatic acyl. In some embodiments, R₂₂ is substituted C₁-C₁₀ aliphatic acyl. In some embodiments, R₂₂ is unsubstituted C₆-C₁₀ aromatic acyl. In some embodiments, R₂₂ is substituted C₆-C₁₀ aromatic acyl. In some embodiments, R₂₂ is unsubstituted C₆-C₁₀ aralkyl acyl. In some embodiments, R₂₂ is substituted C₆-C₁₀ aralkyl acyl. In some embodiments, R₂₂ is unsubstituted C₆-C₁₀ alkylaryl acyl. In some embodiments, R₂₂ is substituted C₆-C₁₀ alkylaryl acyl. In some embodiments, R₂₂ is unsubstituted alkoxy. In some embodiments, R₂₂ is substituted alkoxy. In some embodiments, R₂₂ is unsubstituted aryl. In some embodiments, R₂₂ is substituted aryl. In some embodiments, R₁₈ is unsubstituted C₃-C₁₀heterocyclyl. In some embodiments, R₂₂ is substituted C₃-C₁₀heterocyclyl. In some embodiments, R₂₂ is unsubstituted heteroaryl. In some embodiments, R₂₂ is substituted heteroaryl. In some embodiments, R₂₂ is unsubstituted C₃-C₁₀cycloalkyl. In some embodiments, R₂₂ is substituted C₃-C₁₀cycloalkyl. In some embodiments, R₂₂ is —OPO₃WY. In some embodiments, R₂₂ is —OCH₂PO₄WY. In some embodiments, R₂₂ is —OCH₂PO₄Z. In some embodiments, R₂₂ is —OPO₃Z.

In some embodiments, n is an integer of 0. In some embodiments, n is an integer of 1. In some embodiments, n is an integer of 2. In some embodiments, n is an integer of 3. In some embodiments, n is an integer of 4.

In some embodiments, t is an integer of 0. In some embodiments, t is an integer of 1. In some embodiments, t is an integer of 2. In some embodiments, t is an integer of 3. In some embodiments, t is an integer of 4.

In various embodiments, W is hydrogen. In various embodiments, W is unsubstituted methyl. In various embodiments, W is substituted methyl. In various embodiments, W is unsubstituted ethyl. In various embodiments, W is substituted ethyl. In various embodiments, W is unsubstituted alkyl. In various embodiments, W is substituted alkyl. In various embodiments, W is unsubstituted carbohydrate. In various embodiments, W is substituted carbohydrate. In various embodiments, W is potassium. In various embodiments, W is sodium. In various embodiments, W is lithium.

In various embodiments, Y is hydrogen. In various embodiments, Y is unsubstituted methyl. In various embodiments, Y is substituted methyl. In various embodiments, Y is unsubstituted ethyl. In various embodiments, Y is substituted ethyl. In various embodiments, Y is unsubstituted alkyl. In various embodiments, Y is substituted alkyl. In various embodiments, Y is unsubstituted carbohydrate. In various embodiments, Y is substituted carbohydrate. In various embodiments, Y is potassium. In various embodiments, Y is sodium. In various embodiments, Y is lithium.

In various embodiments, Z is calcium. In various embodiments, Z is magnesium. In various embodiments, Z is iron.

In some embodiments, the pyrone analog of the invention is a compound of Formula XIX or a pharmaceutically/veterinarily acceptable salt thereof:

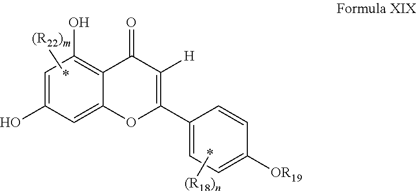

wherein each instance of R₁₈ and R₂₂ is independently hydrogen, hydroxyl, carboxaldehyde, amine, C₁-C₁₀ alkyl C₂-C₁₀ alkynyl, C₂-C₁₀ alkenyl, carboxyl, carbohydrate, ester, acyloxy, nitro, halogen, C₁-C₁₀ aliphatic acyl, C₆-C₁₀ aromatic acyl, C₆-C₁₀ aralkyl acyl, C₆-C₁₀ alkylaryl acyl, alkoxy, alkyl phosphate, aryl, heteroaryl, C₃-C₁₀ heterocyclic, C₃-C₁₀cycloalkyl, —OPO₃WY, —OCH₂PO₄WY, —OCH₂PO₄Z or —OPO₃Z;

R₁₉ is hydrogen, C₁-C₁₀ alkyl C₂-C₁₀ alkynyl, C₂-C₁₀ alkenyl, carbohydrate, C₁-C₁₀ aliphatic acyl, C₆-C₁₀ aromatic acyl, C₆-C₁₀ aralkyl acyl, C₆-C₁₀ alkylaryl acyl, aryl, C₃-C₁₀heterocyclyl, heteroaryl, optionally substituted C₃-C₁₀cycloalkyl, —PO₃WY, —CH₂PO₄WY, —CH₂PO₄Z or —PO₃Z;

n is an integer of 0, 1, 2, 3, or 4;

m is an integer of 0, 1, or 2; and

W and Y are independently hydrogen, methyl, ethyl, alkyl carbohydrate, or a cation, and Z is a multivalent cation, and

wherein the compound of Formula XIX is not one of the following compounds:

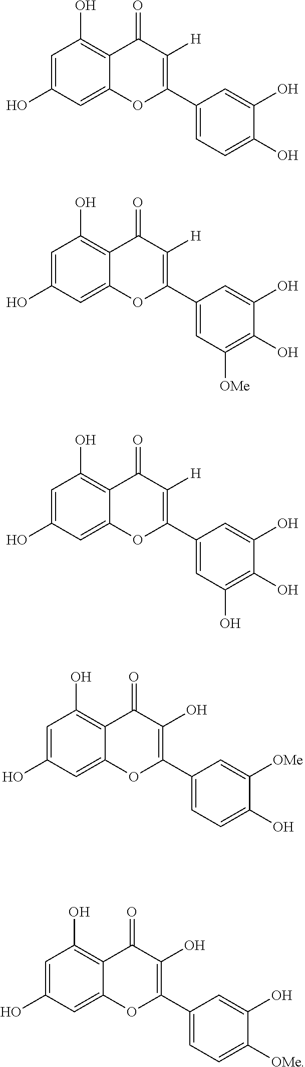

In some embodiments of the invention, R₁₉ is —PO₃WY, —CH₂PO₄WY, —CH₂PO₄Z or —PO₃Z, or at least one of R₁₈ is —OPO₃WY, —OCH₂PO₄WY, —OCH₂PO₄Z or —OPO₃Z. In some embodiments, R₁₉ is —PO₃WY, —CH₂PO₄WY, —CH₂PO₄Z or —PO₃Z and at least one of R₁₈ is —OPO₃WY, —OCH₂PO₄WY, —OCH₂PO₄Z or —OPO₃Z.

In some embodiments, R₁₈ is hydrogen. In some embodiments, R₁₈ is hydroxy. In some embodiments, R₁₈ is carboxaldehyde. In some embodiments, R₁₈ is unsubstituted amine. In some embodiments, R₁₈ is substituted amine. In some embodiments, R₁₈ is unsubstituted C₁-C₁₀ alkyl. In some embodiments, R₁₈ is unsubstituted C₂-C₁₀ alkynyl. In some embodiments, R₁₈ is substituted C₂-C₁₀ alkynyl. In some embodiments, R₁₈ is unsubstituted C₂-C₁₀ alkenyl. In some embodiments, R₁₈ is substituted C₂-C₁₀ alkenyl. In some embodiments, R₁₈ is carboxyl. In some embodiments, R₁₈ is unsubstituted carbohydrate. In some embodiments, R₁₈ is substituted carbohydrate. In some embodiments, R₁₈ is substituted carbohydrate. In some embodiments, R₁₈ is unsubstituted ester. In some embodiments, R₁₈ is substituted ester. In some embodiments, R₁₈ is unsubstituted acyloxy. In some embodiments, R₁₈ is substituted acyloxy. In some embodiments, R₁₈ is nitro. In some embodiments, R₁₈ is halogen. In some embodiments, R₁₈ is unsubstituted C₁-C₁₀ aliphatic acyl. In some embodiments, R₁₈ is substituted C₁-C₁₀ aliphatic acyl. In some embodiments, R₁₈ is unsubstituted C₆-C₁₀ aromatic acyl. In some embodiments, R₁₈ is substituted C₆-C₁₀ aromatic acyl. In some embodiments, R₁₈ is unsubstituted C₆-C₁₀ aralkyl acyl. In some embodiments, R₁₈ is substituted C₆-C₁₀ aralkyl acyl. In some embodiments, R₁₈ is unsubstituted C₆-C₁₀ alkylaryl acyl. In some embodiments, R₁₈ is substituted C₆-C₁₀ alkylaryl acyl. In some embodiments, R₁₈ is unsubstituted alkoxy. In some embodiments, R₁₈ is substituted alkoxy. In some embodiments, R₁₈ is unsubstituted aryl. In some embodiments, R₁₈ is substituted aryl. In some embodiments, R₁₈ is unsubstituted C₃-C₁₀heterocyclyl. In some embodiments, R₁₈ is substituted C₃-C₁₀heterocyclyl. In some embodiments, R₁₈ is unsubstituted heteroaryl. In some embodiments, R₁₈ is substituted heteroaryl. In some embodiments, R₁₈ is unsubstituted C₃-C₁₀cycloalkyl. In some embodiments, R₁₈ is substituted C₃-C₁₀cycloalkyl. In some embodiments, R₁₈ is —OPO₃WY. In some embodiments, R₁₈ is —OCH₂PO₄WY. In some embodiments, R₁₈ is —OCH₂PO₄Z. In some embodiments, R₁₈ is —OPO₃Z.

In some embodiments, R₁₉ is hydrogen. In some embodiments, R₁₉ is unsubstituted C₁-C₁₀ alkyl. In some embodiments, R₁₉ is substituted C₁-C₁₀ alkyl. In some embodiments, R₁₉ is unsubstituted C₂-C₁₀ alkynyl. In some embodiments, R₁₉ is substituted C₂-C₁₀ alkynyl. In some embodiments, R₁₉ is unsubstituted C₂-C₁₀ alkenyl. In some embodiments, R₁₉ is substituted C₂-C₁₀ alkenyl. In some embodiments, R₁₉ is unsubstituted carbohydrate. In some embodiments, R₁₉ is substituted carbohydrate. In some embodiments, R₁₉ is unsubstituted C₁-C₁₀ aliphatic acyl. In some embodiments, R₁₉ is substituted C₁-C₁₀ aliphatic acyl. In some embodiments, R₁₉ is unsubstituted C₆-C₁₀ aromatic acyl. In some embodiments, R₁₉ is substituted C₆-C₁₀ aromatic acyl. In some embodiments, R₁₉ is unsubstituted C₆-C₁₀ aralkyl acyl. In some embodiments, R₁₉ is substituted C₆-C₁₀ aralkyl acyl. In some embodiments, R₁₉ is unsubstituted C₆-C₁₀ alkylaryl acyl. In some embodiments, R₁₉ is substituted C₆-C₁₀ alkylaryl acyl. In some embodiments, R₁₉ is unsubstituted aryl. In some embodiments, R₁₉ is substituted aryl. In some embodiments, R₁₉ is unsubstituted C₃-C₁₀heterocyclyl. In some embodiments, R₁₉ is substituted C₃-C₁₀ heterocyclyl. In some embodiments, R₁₉ is unsubstituted heteroaryl. In some embodiments, R₁₉ is substituted heteroaryl. In some embodiments, R₁₉ is unsubstituted C₃-C₁₀cycloalkyl. In some embodiments, R₁₉ is substituted C₃-C₁₀cycloalkyl. In some embodiments, R₁₉ is —PO₃WY. In some embodiments, R₁₉ is —CH₂PO₄WY. In some embodiments, R₁₉ is —CH₂PO₄Z. In some embodiments, R₁₉ is —PO₃Z.

In some embodiments, R₂₂ is hydrogen. In some embodiments, R₂₂ is hydroxy. In some embodiments, R₂₂ is carboxaldehyde. In some embodiments, R₂₂ is unsubstituted amine. In some embodiments, R₂₂ is substituted amine. In some embodiments, R₂₂ is unsubstituted C₁-C₁₀ alkyl. In some embodiments, R₂₂ is unsubstituted C₂-C₁₀ alkynyl. In some embodiments, R₂₂ is substituted C₂-C₁₀ alkynyl. In some embodiments, R₂₂ is unsubstituted C₂-C₁₀ alkenyl. In some embodiments, R₂₂ is substituted C₂-C₁₀ alkenyl. In some embodiments, R₂₂ is carboxyl. In some embodiments, R₂₂ is unsubstituted carbohydrate. In some embodiments, R₂₂ is substituted carbohydrate. In some embodiments, R₂₂ is unsubstituted ester. In some embodiments, R₂₂ is substituted ester. In some embodiments, R₂₂ is unsubstituted acyloxy. In some embodiments, R₂₂ is substituted acyloxy. In some embodiments, R₂₂ is nitro. In some embodiments, R₂₂ is halogen. In some embodiments, R₂₂ is unsubstituted C₁-C₁₀ aliphatic acyl. In some embodiments, R₂₂ is substituted C₁-C₁₀ aliphatic acyl. In some embodiments, R₂₂ is unsubstituted C₆-C₁₀ aromatic acyl. In some embodiments, R₂₂ is substituted C₆-C₁₀ aromatic acyl. In some embodiments, R₂₂ is unsubstituted C₆-C₁₀ aralkyl acyl. In some embodiments, R₂₂ is substituted C₆-C₁₀ aralkyl acyl. In some embodiments, R₂₂ is unsubstituted C₆-C₁₀ alkylaryl acyl. In some embodiments, R₂₂ is substituted C₆-C₁₀ alkylaryl acyl. In some embodiments, R₂₂ is unsubstituted alkoxy. In some embodiments, R₂₂ is substituted alkoxy. In some embodiments, R₂₂ is unsubstituted aryl. In some embodiments, R₂₂ is substituted aryl. In some embodiments, R₁₈ is unsubstituted C₃-C₁₀heterocyclyl. In some embodiments, R₂₂ is substituted C₃-C₁₀heterocyclyl. In some embodiments, R₂₂ is unsubstituted heteroaryl. In some embodiments, R₂₂ is substituted heteroaryl. In some embodiments, R₂₂ is unsubstituted C₃-C₁₀cycloalkyl. In some embodiments, R₂₂ is substituted C₃-C₁₀cycloalkyl. In some embodiments, R₂₂ is —OPO₃WY. In some embodiments, R₂₂ is —OCH₂PO₄WY. In some embodiments, R₂₂ is —OCH₂PO₄Z. In some embodiments, R₂₂ is —OPO₃Z.

In some embodiments, n is an integer of 0. In some embodiments, n is an integer of 1. In some embodiments, n is an integer of 2. In some embodiments, n is an integer of 3. In some embodiments, n is an integer of 4.

In some embodiments, m is an integer of 0. In some embodiments, m is an integer of 1. In some embodiments, m is an integer of 2.

In various embodiments, W is hydrogen. In various embodiments, W is unsubstituted methyl. In various embodiments, W is substituted methyl. In various embodiments, W is unsubstituted ethyl. In various embodiments, W is substituted ethyl. In various embodiments, W is unsubstituted alkyl. In various embodiments, W is substituted alkyl. In various embodiments, W is unsubstituted carbohydrate. In various embodiments, W is substituted carbohydrate. In various embodiments, W is potassium. In various embodiments, W is sodium. In various embodiments, W is lithium.

In various embodiments, Y is hydrogen. In various embodiments, Y is unsubstituted methyl. In various embodiments, Y is substituted methyl. In various embodiments, Y is unsubstituted ethyl. In various embodiments, Y is substituted ethyl. In various embodiments, Y is unsubstituted alkyl. In various embodiments, Y is substituted alkyl. In various embodiments, Y is unsubstituted carbohydrate. In various embodiments, Y is substituted carbohydrate. In various embodiments, Y is potassium. In various embodiments, Y is sodium. In various embodiments, Y is lithium.

In various embodiments, Z is calcium. In various embodiments, Z is magnesium. In various embodiments, Z is iron.

In some embodiments, the pyrone analog of the invention is a compound of Formula XX or a pharmaceutically/veterinarily acceptable salt thereof:

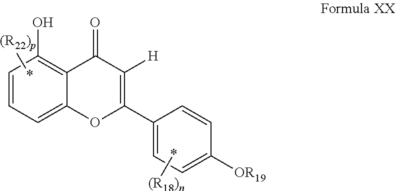

wherein each instance of R₁₈ and R₂₂ is independently hydrogen, hydroxyl, carboxaldehyde, amine, C₁-C₁₀ alkyl, C₂-C₁₀ alkynyl, C₂-C₁₀ alkenyl, carboxyl, carbohydrate, ester, acyloxy, nitro, halogen, C₁-C₁₀ aliphatic acyl, C₆-C₁₀ aromatic acyl, C₆-C₁₀ aralkyl acyl, C₆-C₁₀ alkylaryl acyl, alkoxy, alkyl, phosphate, aryl, heteroaryl, C₃-C₁₀ heterocyclic, C₃-C₁₀cycloalkyl, —OPO₃WY, —OCH₂PO₄WY, —OCH₂PO₄Z or —OPO₃Z;

R₁₉ is hydrogen, C₁-C₁₀ alkyl, C₂-C₁₀ alkynyl, C₂-C₁₀ alkenyl, carbohydrate, C₁-C₁₀ aliphatic acyl, C₆-C₁₀ aromatic acyl, C₆-C₁₀ aralkyl acyl, C₆-C₁₀ alkylaryl acyl, aryl, C₃-C₁₀heterocyclyl, heteroaryl, optionally substituted C₃-C₁₀cycloalkyl, —PO₃WY, —CH₂PO₄WY, —CH₂PO₄Z or —PO₃Z;

n is an integer of 0, 1, 2, 3, or 4;

p is an integer of 0, 1, 2 or 3;

W and Y are independently hydrogen, methyl, ethyl, alkyl, carbohydrate, or a cation, and Z is a multivalent cation; and

wherein the compound of Formula XX is not one of the following compounds:

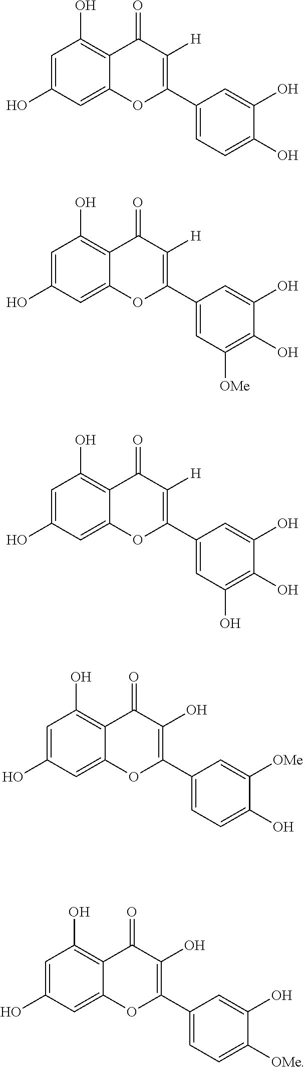

In some embodiments of the invention, R₁₉ is —PO₃WY, —CH₂PO₄WY, —CH₂PO₄Z or —PO₃Z, or at least one of R₁₈ is —OPO₃WY, —OCH₂PO₄WY, —OCH₂PO₄Z or —OPO₃Z. In some embodiments, R₁₉ is —PO₃WY, —CH₂PO₄WY, —CH₂PO₄Z or —PO₃Z and at least one of R₁₈ is —OPO₃WY, —OCH₂PO₄WY, —OCH₂PO₄Z or —OPO₃Z.

In some embodiments, R₁₈ is hydrogen. In some embodiments, R₁₈ is hydroxy. In some embodiments, R₁₈ is carboxaldehyde. In some embodiments, R₁₈ is unsubstituted amine. In some embodiments, R₁₈ is substituted amine. In some embodiments, R₁₈ is unsubstituted C₁-C₁₀ alkyl. In some embodiments, R₁₈ is unsubstituted C₂-C₁₀ alkynyl. In some embodiments, R₁₈ is substituted C₂-C₁₀ alkynyl. In some embodiments, R₁₈ is unsubstituted C₂-C₁₀ alkenyl. In some embodiments, R₁₈ is substituted C₂-C₁₀ alkenyl. In some embodiments, R₁₈ is carboxyl. In some embodiments, R₁₈ is unsubstituted carbohydrate. In some embodiments, R₁₈ is substituted carbohydrate. In some embodiments, R₁₈ is substituted carbohydrate. In some embodiments, R₁₈ is unsubstituted ester. In some embodiments, R₁₈ is substituted ester. In some embodiments, R₁₈ is unsubstituted acyloxy. In some embodiments, R₁₈ is substituted acyloxy. In some embodiments, R₁₈ is nitro. In some embodiments, R₁₈ is halogen. In some embodiments, R₁₈ is unsubstituted C₁-C₁₀ aliphatic acyl. In some embodiments, R₁₈ is substituted C₁-C₁₀ aliphatic acyl. In some embodiments, R₁₈ is unsubstituted C₆-C₁₀ aromatic acyl. In some embodiments, R₁₈ is substituted C₆-C₁₀ aromatic acyl. In some embodiments, R₁₈ is unsubstituted C₆-C₁₀ aralkyl acyl. In some embodiments, R₁₈ is substituted C₆-C₁₀ aralkyl acyl. In some embodiments, R₁₈ is unsubstituted C₆-C₁₀ alkylaryl acyl. In some embodiments, R₁₈ is substituted C₆-C₁₀ alkylaryl acyl. In some embodiments, R₁₈ is unsubstituted alkoxy. In some embodiments, R₁₈ is substituted alkoxy. In some embodiments, R₁₈ is unsubstituted aryl. In some embodiments, R₁₈ is substituted aryl. In some embodiments, R₁₈ is unsubstituted C₃-C₁₀heterocyclyl. In some embodiments, R₁₈ is substituted C₃-C₁₀heterocyclyl. In some embodiments, R₁₈ is unsubstituted heteroaryl. In some embodiments, R₁₈ is substituted heteroaryl. In some embodiments, R₁₈ is unsubstituted C₃-C₁₀cycloalkyl. In some embodiments, R₁₈ is substituted C₃-C₁₀cycloalkyl. In some embodiments, R₁₈ is —OPO₃WY. In some embodiments, R₁₈ is —OCH₂PO₄WY. In some embodiments, R₁₈ is —OCH₂PO₄Z. In some embodiments, R₁₈ is —OPO₃Z.

In some embodiments, R₁₉ is hydrogen. In some embodiments, R₁₉ is unsubstituted C₁-C₁₀ alkyl. In some embodiments, R₁₉ is substituted C₁-C₁₀ alkyl. In some embodiments, R₁₉ is unsubstituted C₂-C₁₀ alkynyl. In some embodiments, R₁₉ is substituted C₂-C₁₀ alkynyl. In some embodiments, R₁₉ is unsubstituted C₂-C₁₀ alkenyl. In some embodiments, R₁₉ is substituted C₂-C₁₀ alkenyl. In some embodiments, R₁₉ is unsubstituted carbohydrate. In some embodiments, R₁₉ is substituted carbohydrate. In some embodiments, R₁₉ is unsubstituted C₁-C₁₀ aliphatic acyl. In some embodiments, R₁₉ is substituted C₁-C₁₀ aliphatic acyl. In some embodiments, R₁₉ is unsubstituted C₆-C₁₀ aromatic acyl. In some embodiments, R₁₉ is substituted C₆-C₁₀ aromatic acyl. In some embodiments, R₁₉ is unsubstituted C₆-C₁₀ aralkyl acyl. In some embodiments, R₁₉ is substituted C₆-C₁₀ aralkyl acyl. In some embodiments, R₁₉ is unsubstituted C₆-C₁₀ alkylaryl acyl. In some embodiments, R₁₉ is substituted C₆-C₁₀ alkylaryl acyl. In some embodiments, R₁₉ is unsubstituted aryl. In some embodiments, R₁₉ is substituted aryl. In some embodiments, R₁₉ is unsubstituted C₃-C₁₀heterocyclyl. In some embodiments, R₁₉ is substituted C₃-C₁₀ heterocyclyl. In some embodiments, R₁₉ is unsubstituted heteroaryl. In some embodiments, R₁₉ is substituted heteroaryl. In some embodiments, R₁₉ is unsubstituted C₃-C₁₀cycloalkyl. In some embodiments, R₁₉ is substituted C₃-C₁₀cycloalkyl. In some embodiments, R₁₉ is —PO₃WY. In some embodiments, R₁₉ is —CH₂PO₄WY. In some embodiments, R₁₉ is —CH₂PO₄Z. In some embodiments, R₁₉ is —PO₃Z.

In some embodiments, R₂₂ is hydrogen. In some embodiments, R₂₂ is hydroxy. In some embodiments, R₂₂ is carboxaldehyde. In some embodiments, R₂₂ is unsubstituted amine. In some embodiments, R₂₂ is substituted amine. In some embodiments, R₂₂ is unsubstituted C₁-C₁₀ alkyl. In some embodiments, R₂₂ is unsubstituted C₂-C₁₀ alkynyl. In some embodiments, R₂₂ is substituted C₂-C₁₀ alkynyl. In some embodiments, R₂₂ is unsubstituted C₂-C₁₀ alkenyl. In some embodiments, R₂₂ is substituted C₂-C₁₀ alkenyl. In some embodiments, R₂₂ is carboxyl. In some embodiments, R₂₂ is unsubstituted carbohydrate. In some embodiments, R₂₂ is substituted carbohydrate. In some embodiments, R₂₂ is unsubstituted ester. In some embodiments, R₂₂ is substituted ester. In some embodiments, R₂₂ is unsubstituted acyloxy. In some embodiments, R₂₂ is substituted acyloxy. In some embodiments, R₂₂ is nitro. In some embodiments, R₂₂ is halogen. In some embodiments, R₂₂ is unsubstituted C₁-C₁₀ aliphatic acyl. In some embodiments, R₂₂ is substituted C₁-C₁₀ aliphatic acyl. In some embodiments, R₂₂ is unsubstituted C₆-C₁₀ aromatic acyl. In some embodiments, R₂₂ is substituted C₆-C₁₀ aromatic acyl. In some embodiments, R₂₂ is unsubstituted C₆-C₁₀ aralkyl acyl. In some embodiments, R₂₂ is substituted C₆-C₁₀ aralkyl acyl. In some embodiments, R₂₂ is unsubstituted C₆-C₁₀ alkylaryl acyl. In some embodiments, R₂₂ is substituted C₆-C₁₀ alkylaryl acyl. In some embodiments, R₂₂ is unsubstituted alkoxy. In some embodiments, R₂₂ is substituted alkoxy. In some embodiments, R₂₂ is unsubstituted aryl. In some embodiments, R₂₂ is substituted aryl. In some embodiments, R₁₈ is unsubstituted C₃-C₁₀heterocyclyl. In some embodiments, R₂₂ is substituted C₃-C₁₀heterocyclyl. In some embodiments, R₂₂ is unsubstituted heteroaryl. In some embodiments, R₂₂ is substituted heteroaryl. In some embodiments, R₂₂ is unsubstituted C₃-C₁₀cycloalkyl. In some embodiments, R₂₂ is substituted C₃-C₁₀cycloalkyl. In some embodiments, R₂₂ is —OPO₃WY. In some embodiments, R₂₂ is —OCH₂PO₄WY. In some embodiments, R₂₂ is —OCH₂PO₄Z. In some embodiments, R₂₂ is —OPO₃Z.

In some embodiments, n is an integer of 0. In some embodiments, n is an integer of 1. In some embodiments, n is an integer of 2. In some embodiments, n is an integer of 3. In some embodiments, n is an integer of 4.

In some embodiments, p is an integer of 0. In some embodiments, p is an integer of 1. In some embodiments, p is an integer of 2. In some embodiments, p is an integer of 3.

In various embodiments, W is hydrogen. In various embodiments, W is unsubstituted methyl. In various embodiments, W is substituted methyl. In various embodiments, W is unsubstituted ethyl. In various embodiments, W is substituted ethyl. In various embodiments, W is unsubstituted alkyl. In various embodiments, W is substituted alkyl. In various embodiments, W is unsubstituted carbohydrate. In various embodiments, W is substituted carbohydrate. In various embodiments, W is potassium. In various embodiments, W is sodium. In various embodiments, W is lithium.

In various embodiments, Y is hydrogen. In various embodiments, Y is unsubstituted methyl. In various embodiments, Y is substituted methyl. In various embodiments, Y is unsubstituted ethyl. In various embodiments, Y is substituted ethyl. In various embodiments, Y is unsubstituted alkyl. In various embodiments, Y is substituted alkyl. In various embodiments, Y is unsubstituted carbohydrate. In various embodiments, Y is substituted carbohydrate. In various embodiments, Y is potassium. In various embodiments, Y is sodium. In various embodiments, Y is lithium.

In various embodiments, Z is calcium. In various embodiments, Z is magnesium. In various embodiments, Z is iron.

In some embodiments, the pyrone analog of the invention is a compound of Formula XXI or a pharmaceutically/veterinarily acceptable salt thereof:

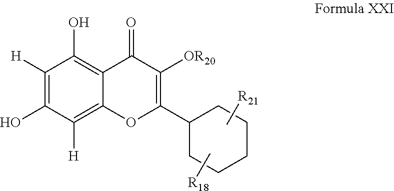

wherein R₁₈ and R₂₁ are independently hydrogen, hydroxyl, carboxaldehyde, amine, C₁-C₁₀ alkyl, C₂-C₁₀ alkynyl, C₂-C₁₀ alkenyl, carboxyl, carbohydrate, ester, acyloxy, nitro, halogen, C₁-C₁₀ aliphatic acyl, C₆-C₁₀ aromatic acyl, C₆-C₁₀ aralkyl acyl, C₆-C₁₀alkylaryl acyl, alkoxy, alkyl, phosphate, aryl, heteroaryl, C₃-C₁₀ heterocyclic, C₃-C₁₀cycloalkyl, —OPO₃WY, —OCH₂PO₄WY, —OCH₂PO₄Z or —OPO₃Z;

R₂₀ is hydrogen, C₁-C₁₀ alkyl, C₂-C₁₀ alkynyl, C₂-C₁₀ alkenyl, carbohydrate, C₁-C₁₀ aliphatic acyl, C₆-C₁₀ aromatic acyl, C₆-C₁₀ aralkyl acyl, C₆-C₁₀ alkylaryl acyl, aryl, C₃-C₁₀ heterocyclyl, heteroaryl, optionally substituted C₃-C₁₀cycloalkyl, —PO₃WY, —CH₂PO₄WY, —CH₂PO₄Z or —PO₃Z; and

W and Y are independently hydrogen, methyl, ethyl, alkyl, carbohydrate, or a cation, and Z is a multivalent cation.

In some embodiments of the invention, at least one of R₁₈₇ or R₂₁ is —OPO₃WY, —OCH₂PO₄WY, —OCH₂PO₄Z or —OPO₃Z.

In some embodiments, R₁₈ is hydrogen. In some embodiments, R₁₈ is hydroxy. In some embodiments, R₁₈ is carboxaldehyde. In some embodiments, R₁₈ is unsubstituted amine. In some embodiments, R₁₈ is substituted amine. In some embodiments, R₁₈ is unsubstituted C₁-C₁₀ alkyl. In some embodiments, R₁₈ is unsubstituted C₂-C₁₀ alkynyl. In some embodiments, R₁₈ is substituted C₂-C₁₀ alkynyl. In some embodiments, R₁₈ is unsubstituted C₂-C₁₀ alkenyl. In some embodiments, R₁₈ is substituted C₂-C₁₀ alkenyl. In some embodiments, R₁₈ is carboxyl. In some embodiments, R₁₈ is unsubstituted carbohydrate. In some embodiments, R₁₈ is substituted carbohydrate. In some embodiments, R₁₈ is substituted carbohydrate. In some embodiments, R₁₈ is unsubstituted ester. In some embodiments, R₁₈ is substituted ester. In some embodiments, R₁₈ is unsubstituted acyloxy. In some embodiments, R₁₈ is substituted acyloxy. In some embodiments, R₁₈ is nitro. In some embodiments, R₁₈ is halogen. In some embodiments, R₁₈ is unsubstituted C₁-C₁₀ aliphatic acyl. In some embodiments, R₁₈ is substituted C₁-C₁₀ aliphatic acyl. In some embodiments, R₁₈ is unsubstituted C₆-C₁₀ aromatic acyl. In some embodiments, R₁₈ is substituted C₆-C₁₀ aromatic acyl. In some embodiments, R₁₈ is unsubstituted C₆-C₁₀ aralkyl acyl. In some embodiments, R₁₈ is substituted C₆-C₁₀ aralkyl acyl. In some embodiments, R₁₈ is unsubstituted C₆-C₁₀ alkylaryl acyl. In some embodiments, R₁₈ is substituted C₆-C₁₀ alkylaryl acyl. In some embodiments, R₁₈ is unsubstituted alkoxy. In some embodiments, R₁₈ is substituted alkoxy. In some embodiments, R₁₈ is unsubstituted aryl. In some embodiments, R₁₈ is substituted aryl. In some embodiments, R₁₈ is unsubstituted C₃-C₁₀heterocyclyl. In some embodiments, R₁₈ is substituted C₃-C₁₀heterocyclyl. In some embodiments, R₁₈ is unsubstituted heteroaryl. In some embodiments, R₁₈ is substituted heteroaryl. In some embodiments, R₁₈ is unsubstituted C₃-C₁₀cycloalkyl. In some embodiments, R₁₈ is substituted C₃-C₁₀cycloalkyl. In some embodiments, R₁₈ is —OPO₃WY. In some embodiments, R₁₈ is —OCH₂PO₄WY. In some embodiments, R₁₈ is —OCH₂PO₄Z. In some embodiments, R₁₈ is —OPO₃Z.

In some embodiments, R₂₀ is hydrogen. In some embodiments, R₂₀ is unsubstituted C₁-C₁₀ alkyl. In some embodiments, R₂₀ is substituted C₁-C₁₀ alkyl. In some embodiments, R₂₀ is unsubstituted C₂-C₁₀ alkynyl. In some embodiments, R₂₀ is substituted C₂-C₁₀ alkynyl. In some embodiments, R₂₀ is unsubstituted C₂-C₁₀ alkenyl. In some embodiments, R₂₀ is substituted C₂-C₁₀ alkenyl. In some embodiments, R₂₀ is unsubstituted carbohydrate. In some embodiments, R₂₀ is substituted carbohydrate. In some embodiments, R₂₀ is unsubstituted C₁-C₁₀ aliphatic acyl. In some embodiments, R₂₀ is substituted C₁-C₁₀ aliphatic acyl. In some embodiments, R₂₀ is unsubstituted C₆-C₁₀ aromatic acyl. In some embodiments, R₂₀ is substituted C₆-C₁₀ aromatic acyl. In some embodiments, R₂₀ is unsubstituted C₆-C₁₀ aralkyl acyl. In some embodiments, R₂₀ is substituted C₆-C₁₀ aralkyl acyl. In some embodiments, R₂₀ is unsubstituted C₆-C₁₀ alkylaryl acyl. In some embodiments, R₂₀ is substituted C₆-C₁₀ alkylaryl acyl. In some embodiments, R₂₀ is unsubstituted aryl. In some embodiments, R₂₀ is substituted aryl. In some embodiments, R₂₀ is unsubstituted C₃-C₁₀heterocyclyl. In some embodiments, R₂₀ is substituted C₃-C₁₀ heterocyclyl. In some embodiments, R₂₀ is unsubstituted heteroaryl. In some embodiments, R₂₀ is substituted heteroaryl. In some embodiments, R₂₀ is unsubstituted C₃-C₁₀cycloalkyl. In some embodiments, R₂₀ is substituted C₃-C₁₀cycloalkyl. In some embodiments, R₂₀ is —PO₃WY. In some embodiments, R₂₀ is —CH₂PO₄WY. In some embodiments, R₂₀ is —CH₂PO₄Z. In some embodiments, R₂₀ is —PO₃Z.

In some embodiments, R₂₁ is hydrogen. In some embodiments, R₂₁ is hydroxy. In some embodiments, R₂₁ is carboxaldehyde. In some embodiments, R₂₁ is unsubstituted amine. In some embodiments, R₂₁ is substituted amine. In some embodiments, R₂₁ is unsubstituted C₁-C₁₀ alkyl. In some embodiments, R₂₁ is unsubstituted C₂-C₁₀ alkynyl. In some embodiments, R₂₁ is substituted C₂-C₁₀ alkynyl. In some embodiments, R₂₁ is unsubstituted C₂-C₁₀ alkenyl. In some embodiments, R₂₁ is substituted C₂-C₁₀ alkenyl. In some embodiments, R₂₁ is carboxyl. In some embodiments, R₂₁ is unsubstituted carbohydrate. In some embodiments, R₂₁ is substituted carbohydrate. In some embodiments, R₂₁ is unsubstituted ester. In some embodiments, R₂₁ is substituted ester. In some embodiments, R₂₁ is unsubstituted acyloxy. In some embodiments, R₂₁ is substituted acyloxy. In some embodiments, R₂₁ is nitro. In some embodiments, R₂₁ is halogen. In some embodiments, R₂₁ is unsubstituted C₁-C₁₀ aliphatic acyl. In some embodiments, R₂₁ is substituted C₁-C₁₀ aliphatic acyl. In some embodiments, R₂₁ is unsubstituted C₆-C₁₀ aromatic acyl. In some embodiments, R₂₁ is substituted C₆-C₁₀ aromatic acyl. In some embodiments, R₂₁ is unsubstituted C₆-C₁₀ aralkyl acyl. In some embodiments, R₂₁ is substituted C₆-C₁₀ aralkyl acyl. In some embodiments, R₂₁ is unsubstituted C₆-C₁₀ alkylaryl acyl. In some embodiments, R₂₁ is substituted C₆-C₁₀ alkylaryl acyl. In some embodiments, R₂₁ is unsubstituted alkoxy. In some embodiments, R₂₁ is substituted alkoxy. In some embodiments, R₂₁ is unsubstituted aryl. In some embodiments, R₂₁ is substituted aryl. In some embodiments, R₂₁ is unsubstituted C₃-C₁₀heterocyclyl. In some embodiments, R₂₁ is substituted C₃-C₁₀heterocyclyl. In some embodiments, R₂₁ is unsubstituted heteroaryl. In some embodiments, R₂₁ is substituted heteroaryl. In some embodiments, R₂₁ is unsubstituted C₃-C₁₀cycloalkyl. In some embodiments, R₂₁ is substituted C₃-C₁₀cycloalkyl. In some embodiments, R₂₁ is —OPO₃WY. In some embodiments, R₂₁ is —OCH₂PO₄WY. In some embodiments, R₂₁ is —OCH₂PO₄Z. In some embodiments, R₂₁ is —OPO₃Z.

In various embodiments, W is hydrogen. In various embodiments, W is unsubstituted methyl. In various embodiments, W is substituted methyl. In various embodiments, W is unsubstituted ethyl. In various embodiments, W is substituted ethyl. In various embodiments, W is unsubstituted alkyl. In various embodiments, W is substituted alkyl. In various embodiments, W is unsubstituted carbohydrate. In various embodiments, W is substituted carbohydrate. In various embodiments, W is potassium. In various embodiments, W is sodium. In various embodiments, W is lithium.

In various embodiments, Y is hydrogen. In various embodiments, Y is unsubstituted methyl. In various embodiments, Y is substituted methyl. In various embodiments, Y is unsubstituted ethyl. In various embodiments, Y is substituted ethyl. In various embodiments, Y is unsubstituted alkyl. In various embodiments, Y is substituted alkyl. In various embodiments, Y is unsubstituted carbohydrate. In various embodiments, Y is substituted carbohydrate. In various embodiments, Y is potassium. In various embodiments, Y is sodium. In various embodiments, Y is lithium.

In various embodiments, Z is calcium. In various embodiments, Z is magnesium. In various embodiments, Z is iron.

In some embodiments, the pyrone analog of the invention is a compound of Formula XXII or a pharmaceutically/veterinarily acceptable salt thereof:

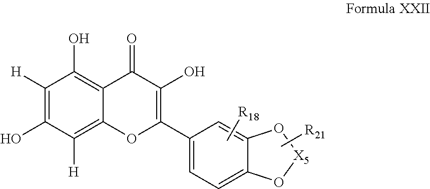

wherein X₅ is a C₁ to C₄ group, optionally interrupted by O, S, NR₂₃, or NR₂₃R₂₃ as valency permits, forming a ring which is aromatic or nonaromatic;

wherein R₁₈ and R₂₁ are independently hydrogen, hydroxyl, carboxaldehyde, amine, C₁-C₁₀ alkyl, C₂-C₁₀ alkynyl, C₂-C₁₀ alkenyl, carboxyl, carbohydrate, ester, acyloxy, nitro, halogen, C₁-C₁₀ aliphatic acyl, C₆-C₁₀ aromatic acyl, C₆-C₁₀ aralkyl acyl, C₆-C₁₀alkylaryl acyl, alkoxy, alkyl, phosphate, aryl, heteroaryl, C₃-C₁₀ heterocyclic, C₃-C₁₀cycloalkyl, —OPO₃WY, —OCH₂PO₄WY, —OCH₂PO₄Z or —OPO₃Z;

each instance of R₂₃ is independently hydrogen, C₁-C₁₀ alkyl, C₂-C₁₀ alkynyl, C₂-C₁₀ alkenyl, carbohydrate, acyloxy, C₁-C₁₀ aliphatic acyl, C₆-C₁₀ aromatic acyl, C₆-C₁₀ aralkyl acyl, C₆-C₁₀ alkylaryl acyl, alkoxy, aryl, heteroaryl, C₅-C₁₀heterocyclyl, C₃-C₁₀cycloalkyl, —PO₃WY, —CH₂PO₄WY, —CH₂PO₄Z or —PO₃Z;

W and Y are independently hydrogen, methyl, ethyl, alkyl, carbohydrate, and a cation; and Z is a multivalent cation.

In some embodiments of the invention, at least one of R₁₈ or R₂₁ is —OPO₃WY, —OCH₂PO₄WY, —OCH₂PO₄Z or —OPO₃Z.

In some embodiments, R₁₈ is hydrogen. In some embodiments, R₁₈ is hydroxy. In some embodiments, R₁₈ is carboxaldehyde. In some embodiments, R₁₈ is unsubstituted amine. In some embodiments, R₁₈ is substituted amine. In some embodiments, R₁₈ is unsubstituted C₁-C₁₀ alkyl. In some embodiments, R₁₈ is unsubstituted C₂-C₁₀ alkynyl. In some embodiments, R₁₈ is substituted C₂-C₁₀ alkynyl. In some embodiments, R₁₈ is unsubstituted C₂-C₁₀ alkenyl. In some embodiments, R₁₈ is substituted C₂-C₁₀ alkenyl. In some embodiments, R₁₈ is carboxyl. In some embodiments, R₁₈ is unsubstituted carbohydrate. In some embodiments, R₁₈ is substituted carbohydrate. In some embodiments, R₁₈ is substituted carbohydrate. In some embodiments, R₁₈ is unsubstituted ester. In some embodiments, R₁₈ is substituted ester. In some embodiments, R₁₈ is unsubstituted acyloxy. In some embodiments, R₁₈ is substituted acyloxy. In some embodiments, R₁₈ is nitro. In some embodiments, R₁₈ is halogen. In some embodiments, R₁₈ is unsubstituted C₁-C₁₀ aliphatic acyl. In some embodiments, R₁₈ is substituted C₁-C₁₀ aliphatic acyl. In some embodiments, R₁₈ is unsubstituted C₆-C₁₀ aromatic acyl. In some embodiments, R₁₈ is substituted C₆-C₁₀ aromatic acyl. In some embodiments, R₁₈ is unsubstituted C₆-C₁₀ aralkyl acyl. In some embodiments, R₁₈ is substituted C₆-C₁₀ aralkyl acyl. In some embodiments, R₁₈ is unsubstituted C₆-C₁₀ alkylaryl acyl. In some embodiments, R₁₈ is substituted C₆-C₁₀ alkylaryl acyl. In some embodiments, R₁₈ is unsubstituted alkoxy. In some embodiments, R₁₈ is substituted alkoxy. In some embodiments, R₁₈ is unsubstituted aryl. In some embodiments, R₁₈ is substituted aryl. In some embodiments, R₁₈ is unsubstituted C₃-C₁₀heterocyclyl. In some embodiments, R₁₈ is substituted C₃-C₁₀heterocyclyl. In some embodiments, R₁₈ is unsubstituted heteroaryl. In some embodiments, R₁₈ is substituted heteroaryl. In some embodiments, R₁₈ is unsubstituted C₃-C₁₀cycloalkyl. In some embodiments, R₁₈ is substituted C₃-C₁₀cycloalkyl. In some embodiments, R₁₈ is —OPO₃WY. In some embodiments, R₁₈ is —OCH₂PO₄WY. In some embodiments, R₁₈ is —OCH₂PO₄Z. In some embodiments, R₁₈ is —OPO₃Z.

In some embodiments, R₂₁ is hydrogen. In some embodiments, R₂₁ is hydroxy. In some embodiments, R₂₁ is carboxaldehyde. In some embodiments, R₂₁ is unsubstituted amine. In some embodiments, R₂₁ is substituted amine. In some embodiments, R₂₁ is unsubstituted C₁-C₁₀ alkyl. In some embodiments, R₂₁ is unsubstituted C₂-C₁₀ alkynyl. In some embodiments, R₂₁ is substituted C₂-C₁₀ alkynyl. In some embodiments, R₂₁ is unsubstituted C₂-C₁₀ alkenyl. In some embodiments, R₂₁ is substituted C₂-C₁₀ alkenyl. In some embodiments, R₂₁ is carboxyl. In some embodiments, R₂₁ is unsubstituted carbohydrate. In some embodiments, R₂₁ is substituted carbohydrate. In some embodiments, R₂₁ is unsubstituted ester. In some embodiments, R₂₁ is substituted ester. In some embodiments, R₂ is unsubstituted acyloxy. In some embodiments, R₂₁ is substituted acyloxy. In some embodiments, R₂₁ is nitro. In some embodiments, R₂₁ is halogen. In some embodiments, R₂₁ is unsubstituted C₁-C₁₀ aliphatic acyl. In some embodiments, R₂₁ is substituted C₁-C₁₀ aliphatic acyl. In some embodiments, R₂₁ is unsubstituted C₆-C₁₀ aromatic acyl. In some embodiments, R₂₁ is substituted C₆-C₁₀ aromatic acyl. In some embodiments, R₂₁ is unsubstituted C₆-C₁₀ aralkyl acyl. In some embodiments, R₂₁ is substituted C₆-C₁₀ aralkyl acyl. In some embodiments, R₂₁ is unsubstituted C₆-C₁₀ alkylaryl acyl. In some embodiments, R₂₁ is substituted C₆-C₁₀ alkylaryl acyl. In some embodiments, R₂₁ is unsubstituted alkoxy. In some embodiments, R₂₁ is substituted alkoxy. In some embodiments, R₂₁ is unsubstituted aryl. In some embodiments, R₂₁ is substituted aryl. In some embodiments, R₂₁ is unsubstituted C₃-C₁₀heterocyclyl. In some embodiments, R₂₁ is substituted C₃-C₁₀heterocyclyl. In some embodiments, R₂₁ is unsubstituted heteroaryl. In some embodiments, R₂₁ is substituted heteroaryl. In some embodiments, R₂₁ is unsubstituted C₃-C₁₀cycloalkyl. In some embodiments, R₂₁ is substituted C₃-C₁₀cycloalkyl. In some embodiments, R₂₁ is —OPO₃WY. In some embodiments, R₂₁ is —OCH₂PO₄WY. In some embodiments, R₂₁ is —OCH₂PO₄Z. In some embodiments, R₂₁ is —OPO₃Z.

In some embodiments, R₂₃ is hydrogen. In some embodiments, R₂₃ is unsubstituted C₁-C₁₀ alkyl. In some embodiments, R₂₃ is substituted C₁-C₁₀ alkyl. In some embodiments, R₂₃ is unsubstituted C₂-C₁₀ alkynyl. In some embodiments, R₂₃ is substituted C₂-C₁₀ alkynyl. In some embodiments, R₂₃ is unsubstituted C₂-C₁₀ alkenyl. In some embodiments, R₂₃ is substituted C₂-C₁₀ alkenyl. In some embodiments, R₂₃ is unsubstituted acyloxy. In some embodiments, R₂₃ is substituted acyloxy. In some embodiments, R₂₃ is unsubstituted carbohydrate. In some embodiments, R₂₃ is substituted carbohydrate. In some embodiments, R₂₃ is unsubstituted acyloxy. In some embodiments, R₂₃ is substituted acyloxy. In some embodiments, R₂₃ is unsubstituted C₁-C₁₀ aliphatic acyl. In some embodiments, R₂₃ is substituted C₁-C₁₀ aliphatic acyl. In some embodiments, R₂₃ is unsubstituted C₆-C₁₀ aromatic acyl. In some embodiments, R₂₃ is substituted C₆-C₁₀ aromatic acyl. In some embodiments, R₂₃ is unsubstituted C₆-C₁₀ aralkyl acyl. In some embodiments, R₂₃ is substituted C₆-C₁₀ aralkyl acyl. In some embodiments, R₂₃ is unsubstituted C₆-C₁₀ alkylaryl acyl. In some embodiments, R₂₃ is substituted C₆-C₁₀ alkylaryl acyl. In some embodiments, R₂₃ is unsubstituted alkoxy. In some embodiments, R₂₃ is substituted alkoxy. In some embodiments, R₂₃ is unsubstituted aryl. In some embodiments, R₂₃ is substituted aryl. In some embodiments, R₂₃ is unsubstituted C₃-C₁₀heterocyclyl. In some embodiments, R₂₃ is substituted C₃-C₁₀heterocyclyl. In some embodiments, R₂₃ is unsubstituted heteroaryl. In some embodiments, R₂₃ is substituted heteroaryl. In some embodiments, R₂₃ is unsubstituted C₃-C₁₀cycloalkyl. In some embodiments, R₂₃ is substituted C₃-C₁₀cycloalkyl.

In various embodiments, W is hydrogen. In various embodiments, W is unsubstituted methyl. In various embodiments, W is substituted methyl. In various embodiments, W is unsubstituted ethyl. In various embodiments, W is substituted ethyl. In various embodiments, W is unsubstituted alkyl. In various embodiments, W is substituted alkyl. In various embodiments, W is unsubstituted carbohydrate. In various embodiments, W is substituted carbohydrate. In various embodiments, W is potassium. In various embodiments, W is sodium. In various embodiments, W is lithium.

In various embodiments, Y is hydrogen. In various embodiments, Y is unsubstituted methyl. In various embodiments, Y is substituted methyl. In various embodiments, Y is unsubstituted ethyl. In various embodiments, Y is substituted ethyl. In various embodiments, Y is unsubstituted alkyl. In various embodiments, Y is substituted alkyl. In various embodiments, Y is unsubstituted carbohydrate. In various embodiments, Y is substituted carbohydrate. In various embodiments, Y is potassium. In various embodiments, Y is sodium. In various embodiments, Y is lithium.

In various embodiments, Z is calcium. In various embodiments, Z is magnesium. In various embodiments, Z is iron.

In some embodiments, the pyrone analog of the invention is a compound of Formula XXIII or a pharmaceutically/veterinarily acceptable salt thereof:

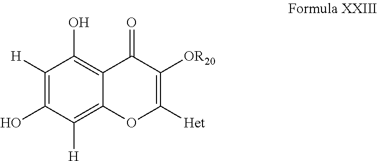

wherein R₂₀ is hydrogen, C₁-C₁₀ alkyl, C₂-C₁₀ alkynyl, C₂-C₁₀ alkenyl, carbohydrate, C₁-C₁₀ aliphatic acyl, C₆-C₁₀ aromatic acyl, C₆-C₁₀ aralkyl acyl, C₆-C₁₀ alkylaryl acyl, aryl, C₃-C₁₀ heterocyclyl, heteroaryl, optionally substituted C₃-C₁₀cycloalkyl, —PO₃WY, —CH₂PO₄WY, —CH₂PO₄Z or —PO₃Z;

Het is a 3 to 10 membered optionally substituted monocyclic or bicyclic heteroaromatic or heteroalicyclic ring system containing 1, 2, 3, 4, or 5 heteroatoms selected from the group of O, S, and N, with the proviso that no two adjacent ring atoms are O or S, wherein the ring system is unsaturated, partially unsaturated or saturated, wherein any number of the ring atoms have substituents as valency permits which are hydrogen, hydroxyl, carboxaldehyde, alkylcarboxaldehyde, imino, C₁-C₁₀ alkyl, C₁-C₁₀ alkynyl, C₁-C₁₀ alkenyl, carboxyl, carbohydrate, acyloxy, nitro, halogen, C₁-C₁₀ aliphatic acyl, C₅-C₁₀ aromatic acyl, C₆-C₁₀ aralkyl acyl, C₆-C₁₀ alkylaryl acyl, alkoxy, amine, aryl, heteroaryl, C₅-C₁₀heterocyclyl, C₅-C₁₀cycloalkyl, —OPO₃WY, —OCH₂PO₄WY, —OCH₂PO₄Z or —OPO₃Z; and

W and Y are independently hydrogen, methyl, ethyl, alkyl, carbohydrate, or a cation, and Z is a multivalent cation.

In some embodiments, R₂₀ is hydrogen. In some embodiments, R₂₀ is unsubstituted C₁-C₁₀ alkyl. In some embodiments, R₂₀ is substituted C₁-C₁₀ alkyl. In some embodiments, R₂₀ is unsubstituted C₂-C₁₀ alkynyl. In some embodiments, R₂₀ is substituted C₂-C₁₀ alkynyl. In some embodiments, R₂₀ is unsubstituted C₂-C₁₀ alkenyl. In some embodiments, R₂₀ is substituted C₂-C₁₀ alkenyl. In some embodiments, R₂₀ is unsubstituted carbohydrate. In some embodiments, R₂₀ is substituted carbohydrate. In some embodiments, R₂₀ is unsubstituted C₁-C₁₀ aliphatic acyl. In some embodiments, R₂₀ is substituted C₁-C₁₀ aliphatic acyl. In some embodiments, R₂₀ is unsubstituted C₆-C₁₀ aromatic acyl. In some embodiments, R₂₀ is substituted C₆-C₁₀ aromatic acyl. In some embodiments, R₂₀ is unsubstituted C₆-C₁₀ aralkyl acyl. In some embodiments, R₂₀ is substituted C₆-C₁₀ aralkyl acyl. In some embodiments, R₂₀ is unsubstituted C₆-C₁₀ alkylaryl acyl. In some embodiments, R₂₀ is substituted C₆-C₁₀ alkylaryl acyl. In some embodiments, R₂₀ is unsubstituted aryl. In some embodiments, R₂₀ is substituted aryl. In some embodiments, R₂₀ is unsubstituted C₃-C₁₀heterocyclyl. In some embodiments, R₂₀ is substituted C₃-C₁₀ heterocyclyl. In some embodiments, R₂₀ is unsubstituted heteroaryl. In some embodiments, R₂₀ is substituted heteroaryl. In some embodiments, R₂₀ is unsubstituted C₃-C₁₀cycloalkyl. In some embodiments, R₂₀ is substituted C₃-C₁₀cycloalkyl. In some embodiments, R₂₀ is —PO₃WY. In some embodiments, R₂₀ is —CH₂PO₄WY. In some embodiments, R₂₀ is —CH₂PO₄Z. In some embodiments, R₂₀ is —PO₃Z.

In some embodiments, Het is one of the following formulae:

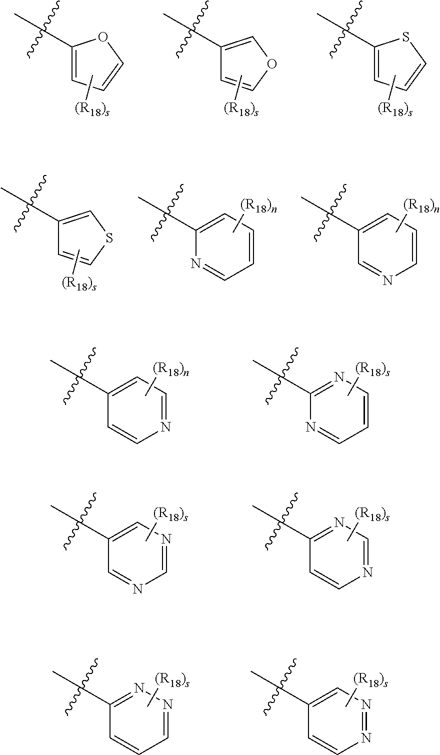

wherein each instance of R₁₈ is independently hydrogen, hydroxyl, carboxaldehyde, amine, C₁-C₁₀ alkyl, C₂-C₁₀ alkynyl, C₂-C₁₀ alkenyl, carboxyl, carbohydrate, ester, acyloxy, nitro, halogen, C₁-C₁₀ aliphatic acyl, C₆-C₁₀ aromatic acyl, C₆-C₁₀ aralkyl acyl, C₆-C₁₀ alkylaryl acyl, alkoxy, alkyl, phosphate, aryl, heteroaryl, C₃-C₁₀ heterocyclic, C₃-C₁₀cycloalkyl, —OPO₃WY, —OCH₂PO₄WY, —OCH₂PO₄Z or —OPO₃Z;

s is an integer of 0, 1, 2, or 3; and

n is an integer of 0, 1, 2, 3, or 4.

In some embodiments of the invention, at least one of R₁₈ is —OPO₃WY, —OCH₂PO₄WY, —OCH₂PO₄Z or —OPO₃Z.

In some embodiments, R₁₈ is hydrogen. In some embodiments, R₁₈ is hydroxy. In some embodiments, R₁₈ is carboxaldehyde. In some embodiments, R₁₈ is unsubstituted amine. In some embodiments, R₁₈ is substituted amine. In some embodiments, R₁₈ is unsubstituted C₁-C₁₀ alkyl. In some embodiments, R₁₈ is unsubstituted C₂-C₁₀ alkynyl. In some embodiments, R₁₈ is substituted C₂-C₁₀ alkynyl. In some embodiments, R₁₈ is unsubstituted C₂-C₁₀ alkenyl. In some embodiments, R₁₈ is substituted C₂-C₁₀ alkenyl. In some embodiments, R₁₈ is carboxyl. In some embodiments, R₁₈ is unsubstituted carbohydrate. In some embodiments, R₁₈ is substituted carbohydrate. In some embodiments, R₁₈ is substituted carbohydrate. In some embodiments, R₁₈ is unsubstituted ester. In some embodiments, R₁₈ is substituted ester. In some embodiments, R₁₈ is unsubstituted acyloxy. In some embodiments, R₁₈ is substituted acyloxy. In some embodiments, R₁₈ is nitro. In some embodiments, R₁₈ is halogen. In some embodiments, R₁₈ is unsubstituted C₁-C₁₀ aliphatic acyl. In some embodiments, R₁₈ is substituted C₁-C₁₀ aliphatic acyl. In some embodiments, R₁₈ is unsubstituted C₆-C₁₀ aromatic acyl. In some embodiments, R₁₈ is substituted C₆-C₁₀ aromatic acyl. In some embodiments, R₁₈ is unsubstituted C₆-C₁₀ aralkyl acyl. In some embodiments, R₁₈ is substituted C₆-C₁₀ aralkyl acyl. In some embodiments, R₁₈ is unsubstituted C₆-C₁₀ alkylaryl acyl. In some embodiments, R₁₈ is substituted C₆-C₁₀ alkylaryl acyl. In some embodiments, R₁₈ is unsubstituted alkoxy. In some embodiments, R₁₈ is substituted alkoxy. In some embodiments, R₁₈ is unsubstituted aryl. In some embodiments, R₁₈ is substituted aryl. In some embodiments, R₁₈ is unsubstituted C₃-C₁₀heterocyclyl. In some embodiments, R₁₈ is substituted C₃-C₁₀heterocyclyl. In some embodiments, R₁₈ is unsubstituted heteroaryl. In some embodiments, R₁₈ is substituted heteroaryl. In some embodiments, R₁₈ is unsubstituted C₃-C₁₀cycloalkyl. In some embodiments, R₁₈ is substituted C₃-C₁₀cycloalkyl. In some embodiments, R₁₈ is —OPO₃WY. In some embodiments, R₁₈ is —OCH₂PO₄WY. In some embodiments, R₁₈ is —OCH₂PO₄Z. In some embodiments, R₁₈ is —OPO₃Z.

In some embodiments, n is an integer of 0. In some embodiments, n is an integer of 1. In some embodiments, n is an integer of 2. In some embodiments, n is an integer of 3. In some embodiments, n is an integer of 4.

In some embodiments, s is an integer of 0. In some embodiments, s is an integer of 1. In some embodiments, s is an integer of 2. In some embodiments, s is an integer of 3.

In various embodiments, W is hydrogen. In various embodiments, W is unsubstituted methyl. In various embodiments, W is substituted methyl. In various embodiments, W is unsubstituted ethyl. In various embodiments, W is substituted ethyl. In various embodiments, W is unsubstituted alkyl. In various embodiments, W is substituted alkyl. In various embodiments, W is unsubstituted carbohydrate. In various embodiments, W is substituted carbohydrate. In various embodiments, W is potassium. In various embodiments, W is sodium. In various embodiments, W is lithium.

In various embodiments, Y is hydrogen. In various embodiments, Y is unsubstituted methyl. In various embodiments, Y is substituted methyl. In various embodiments, Y is unsubstituted ethyl. In various embodiments, Y is substituted ethyl. In various embodiments, Y is unsubstituted alkyl. In various embodiments, Y is substituted alkyl. In various embodiments, Y is unsubstituted carbohydrate. In various embodiments, Y is substituted carbohydrate. In various embodiments, Y is potassium. In various embodiments, Y is sodium. In various embodiments, Y is lithium.

In various embodiments, Z is calcium. In various embodiments, Z is magnesium. In various embodiments, Z is iron.

In some embodiments of the invention, the pyrone analog of Formula II is of Formula IV or a pharmaceutically/veterinarily acceptable salt thereof:

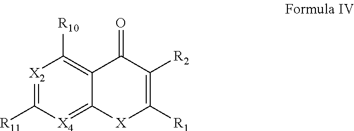

wherein X is O, S, or NR′, wherein R′ is hydrogen, C₁-C₁₀ alkyl, C₂-C₁₀ alkynyl, C₂-C₁₀ alkenyl, C₁-C₁₀ aliphatic acyl, C₆-C₁₀ aromatic acyl, C₆-C₁₀ aralkyl acyl, C₆-C₁₀ alkylaryl acyl, aryl, C₃-C₁₀ heterocyclyl, heteroaryl, or C₃-C₁₀cycloalkyl;

R₁, and R₂ are independently hydrogen, hydroxyl, C₁-C₁₀ alkyl, C₂-C₁₀ alkynyl, C₂-C₁₀ alkenyl, carboxyl, carbohydrate, ester, acyloxy, nitro, halogen, C₁-C₁₀ aliphatic acyl, C₆-C₁₀ aromatic acyl, C₆-C₁₀ aralkyl acyl, C₆-C₁₀ alkylaryl acyl, alkoxy, amine, aryl, C₄-C₁₀heterocyclyl, heteroaryl, C₃-C₁₀cycloalkyl, —OPO₃WY, —OCH₂PO₄WY, —OCH₂PO₄Z or —OPO₃Z;

X₂ and X₄ are independently CR₅, O, S, or N;

each instance of R₅ is independently hydrogen, hydroxyl, carboxaldehyde, amino, C₁-C₁₀ alkyl, C₂-C₁₀ alkynyl, C₂-C₁₀ alkenyl, carboxyl, carbohydrate, ester, acyloxy, nitro, halogen, C₁-C₁₀ aliphatic acyl, C₆-C₁₀ aromatic acyl, C₆-C₁₀ aralkyl acyl, C₆-C₁₀alkylaryl acyl, alkoxy, amine, aryl, C₃-C₁₀heterocyclyl, heteroaryl, C₃-C₁₀cycloalkyl, —OPO₃WY, —OCH₂PO₄WY, —OCH₂PO₄Z or —OPO₃Z;

R₁₀ and R₁₁ are independently hydrogen, hydroxyl, carboxaldehyde, amino, C₁-C₁₀ alkyl, C₂-C₁₀ alkynyl, C₂-C₁₀ alkenyl, carboxyl, carbohydrate, ester, acyloxy, nitro, halogen, C₁-C₁₀ aliphatic acyl, C₆-C₁₀ aromatic acyl, C₆-C₁₀ aralkyl acyl, C₆-C₁₀ alkylaryl acyl, alkoxy, amine, aryl, C₃-C₁₀ heterocyclyl, heteroaryl, C₃-C₁₀cycloalkyl, —OPO₃WY, —OCH₂PO₄WY, —OCH₂PO₄Z or —OPO₃Z;

W and Y are independently hydrogen, methyl, ethyl, alkyl, carbohydrate, or a cation, and Z is a multivalent cation; and

wherein the compound of Formula IV is not the compound of Formula A:

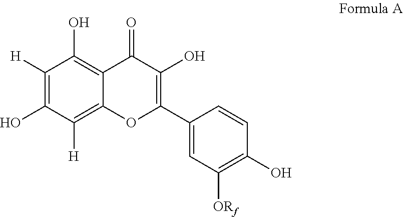

wherein R_{f} is hydrogen.

In some embodiments, the compound of Formula IV is not the compound of Formula A wherein R_{f} is PO₃K₂.

In some embodiments, X is O.

In other embodiments, X is S.

In yet other embodiments, X is NR′.

In some embodiments, R′ is hydrogen. In some embodiments, R′ is unsubstituted C₁-C₁₀ alkyl. In some embodiments, R′ is substituted C₁-C₁₀ alkyl. In some embodiments, R′ is unsubstituted C₂-C₁₀ alkynyl. In some embodiments, R′ is substituted C₂-C₁₀ alkynyl. In some embodiments, R′ is unsubstituted C₂-C₁₀ alkenyl. In some embodiments, R′ is substituted C₂-C₁₀ alkenyl. In some embodiments, R′ is unsubstituted C₂-C₁₀ alkenyl. In some embodiments, R′ is substituted C₂-C₁₀ alkenyl. In some embodiments, R′ is unsubstituted C₁-C₁₀ aliphatic acyl. In some embodiments, R′ is substituted C₁-C₁₀ aliphatic acyl. In some embodiments, R′ is unsubstituted C₆-C₁₀ aromatic acyl. In some embodiments, R′ is substituted C₆-C₁₀ aromatic acyl. In some embodiments, R′ is unsubstituted C₆-C₁₀ aralkyl acyl. In some embodiments, R′ is substituted C₆-C₁₀ aralkyl acyl. In some embodiments, R′ is unsubstituted C₆-C₁₀ alkylaryl acyl. In some embodiments, R′ is substituted C₆-C₁₀ alkylaryl acyl. In some embodiments, R′ is unsubstituted aryl. In some embodiments, R′ is substituted aryl. In some embodiments, R′ is unsubstituted C₃-C₁₀ heterocyclyl. In some embodiments, R′ is substituted C₃-C₁₀ heterocyclyl. In some embodiments, R′ is unsubstituted heteroaryl. In some embodiments, R′ is substituted heteroaryl. In some embodiments, R′ is unsubstituted C₃-C₁₀cycloalkyl. In some embodiments, R′ is substituted C₃-C₁₀cycloalkyl.

In some embodiments, X₂ is CR₅.

In other embodiments, X₂ is O.

In yet other embodiments, X₂ is S.

In further embodiments, X₂ is N.

In other embodiments, X₄ is CR₅.

In some embodiments, X₄ is O.

In yet other embodiments, X₄ is S.

In some embodiments, X₄ is N.

In some embodiments, at least one of X₂ or X₄ is N.

In some embodiments, R₁ is hydrogen. In some embodiments, R₁ is hydroxyl. In some embodiments, R₁ is optionally substituted C₁-C₁₀ alkyl. In some embodiments, R₁ is unsubstituted C₁-C₁₀ alkyl. In some embodiments, R₁ is substituted C₁-C₁₀ alkyl. In some embodiments, R₁ is unsubstituted C₁-C₁₀ alkyl. In some other embodiments, R₁ is substituted C₁-C₁₀ alkyl. In some embodiments, R₁ is unsubstituted C₂-C₁₀ alkynyl. In some embodiments, R₁ is substituted C₂-C₁₀ alkynyl. In some embodiments, R₁ is unsubstituted C₂-C₁₀ alkenyl. In some embodiments, R₁ is substituted C₂-C₁₀ alkenyl. In some embodiments, R₁ is carboxyl. In some embodiments, R₁ is unsubstituted carbohydrate. In some embodiments, R₁ is substituted carbohydrate. In some embodiments, R₁ is unsubstituted ester. In some embodiments, R₁ is substituted ester. In some embodiments, R₁ is unsubstituted acyloxy. In some embodiments, R₁ is substituted acyloxy. In some embodiments, R₁ is nitro. In some embodiments, R₁ is halogen. In some embodiments, R₁ is unsubstituted C₁-C₁₀ aliphatic acyl. In some embodiments, R₁ is substituted C₁-C₁₀ aliphatic acyl. In some embodiments, R₁ is unsubstituted C₆-C₁₀ aromatic acyl. In some embodiments, R₁ is substituted C₆-C₁₀ aromatic acyl. In some embodiments, R₁ is unsubstituted C₆-C₁₀ aralkyl acyl. In some embodiments, R₁ is substituted C₆-C₁₀ aralkyl acyl. In some embodiments, R₁ is unsubstituted C₆-C₁₀ alkylaryl acyl. In some embodiments, R₁ is substituted C₆-C₁₀ alkylaryl acyl. In some embodiments, R₁ is unsubstituted alkoxy. In some embodiments, R₁ is substituted alkoxy. In some embodiments, R₁ is unsubstituted amine. In some embodiments, R₁ is substituted amine. In some embodiments, R₁ is unsubstituted aryl. In some embodiments, R₁ is substituted aryl. In some embodiments, R₁ is unsubstituted C₄-C₁₀heterocyclyl. In some embodiments, R₁ is substituted C₄-C₁₀heterocyclyl. In some embodiments, R₁ is unsubstituted heteroaryl. In some embodiments, R₁ is substituted heteroaryl. In some embodiments, R₁ is unsubstituted C₃-C₁₀cycloalkyl. In some embodiments, R₁ is substituted C₃-C₁₀cycloalkyl. In some embodiments, R₁ is —OPO₃WY. In some embodiments, R₁ is —OCH₂PO₄WY. In some embodiments, R₁ is —OCH₂PO₄Z. In some embodiments, R₁ is —OPO₃Z.

In some embodiments, when R₁ is aryl, it is monocyclic. In some embodiments, when R₁ is aryl, it is bicyclic. In some embodiments, when R₁ is heteroaryl, it is monocyclic. In some embodiments, when R₁ is heteroaryl, it is bicyclic.

In various embodiments, R₁ is one of the following formulae:

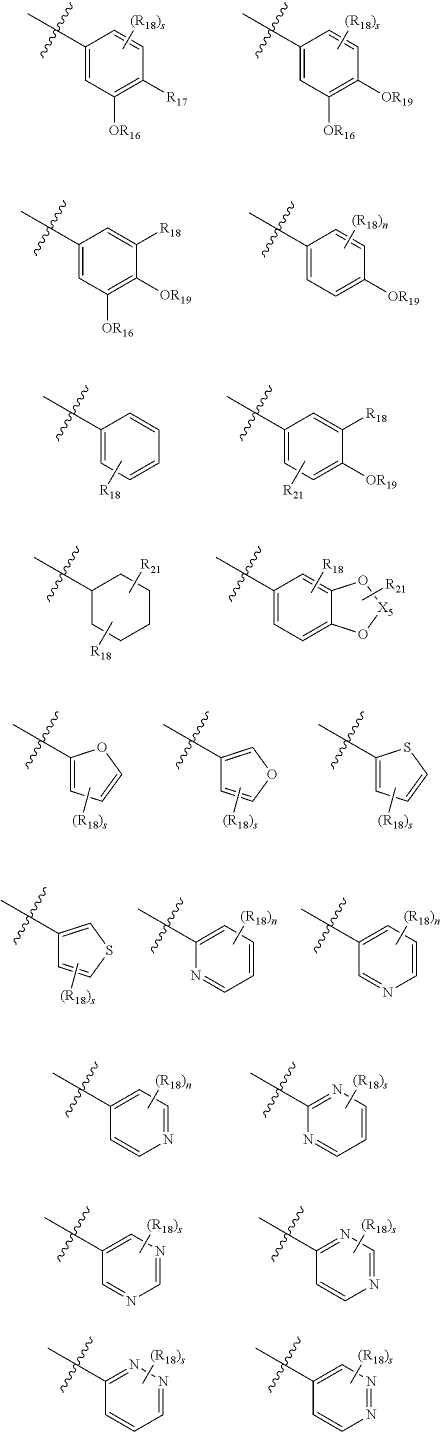

wherein R₁₆ is hydrogen, C₁-C₁₀ alkyl, C₂-C₁₀ alkynyl, C₂-C₁₀ alkenyl, carbohydrate, C₁-C₁₀ aliphatic acyl, C₆-C₁₀ aromatic acyl, C₆-C₁₀ aralkyl acyl, C₆-C₁₀ alkylaryl acyl, aryl, C₃-C₁₀ heterocyclyl, heteroaryl, C₃-C₁₀cycloalkyl, —PO₃WY, —CH₂PO₄WY, —CH₂PO₄Z or —PO₃Z;

R₁₇ is hydrogen, hydroxy, carboxaldehyde, amine, C₁-C₁₀ alkyl, C₂-C₁₀ alkynyl, C₂-C₁₀ alkenyl, carboxyl, carbohydrate, ester, acyloxy, nitro, halogen, C₁-C₁₀ aliphatic acyl, C₆-C₁₀ aromatic acyl, C₆-C₁₀ aralkyl acyl, C₆-C₁₀ alkylaryl acyl, alkoxy, aryl, C₃-C₁₀ heterocyclyl, heteroaryl, or C₃-C₁₀cycloalkyl, —OPO₃WY, —OCH₂PO₄WY, —OCH₂PO₄Z or —OPO₃Z;

each instance of R₁₈ and R₂₁ is independently hydrogen, hydroxyl, carboxaldehyde, amine, C₁-C₁₀ alkyl, C₂-C₁₀ alkynyl, C₂-C₁₀ alkenyl, carboxyl, carbohydrate, ester, acyloxy, nitro, halogen, C₁-C₁₀ aliphatic acyl, C₆-C₁₀ aromatic acyl, C₆-C₁₀ aralkyl acyl, C₆-C₁₀ alkylaryl acyl, alkoxy, alkyl, phosphate, aryl, heteroaryl, C₃-C₁₀ heterocyclic, C₃-C₁₀cycloalkyl, —OPO₃WY, —OCH₂PO₄WY, —OCH₂PO₄Z or —OPO₃Z;

R₁₉ is hydrogen, C₁-C₁₀ alkyl, C₂-C₁₀ alkynyl, C₂-C₁₀ alkenyl, carbohydrate, C₁-C₁₀ aliphatic acyl, C₆-C₁₀ aromatic acyl, C₆-C₁₀ aralkyl acyl, C₆-C₁₀ alkylaryl acyl, aryl, C₃-C₁₀heterocyclyl, heteroaryl, optionally substituted C₃-C₁₀cycloalkyl, —PO₃WY, —CH₂PO₄WY, —CH₂PO₄Z or —PO₃Z;

s is an integer of 0, 1, 2, or 3; and

n is an integer of 0, 1, 2, 3, or 4.

In some embodiments of the invention, at least one of R₁₆ and R₁₉ is —PO₃WY, —CH₂PO₄WY, —CH₂PO₄Z or —PO₃Z, or at least one of R₁₇ or R₁₈ is —OPO₃WY, —OCH₂PO₄WY, —OCH₂PO₄Z or —OPO₃Z. In some embodiments, at least one of R₁₆ and R₁₉ is —PO₃WY, —CH₂PO₄WY, —CH₂PO₄Z or —PO₃Z and at least one of R₁₇ or R₁₈ is —OPO₃WY, —OCH₂PO₄WY, —OCH₂PO₄Z or —OPO₃Z.

In some embodiments, R₂ is hydrogen. In some embodiments, R₂ is hydroxyl. In some embodiments, R₂ is optionally substituted C₁-C₁₀ alkyl. In some embodiments, R₂ is unsubstituted C₁-C₁₀ alkyl. In some embodiments, R₂ is substituted C₁-C₁₀ alkyl. In some embodiments, R₂ is unsubstituted C₁-C₁₀ alkyl. In some other embodiments, R₂ is substituted C₁-C₁₀ alkyl. In some embodiments, R₂ is unsubstituted C₂-C₁₀ alkynyl. In some embodiments, R₂ is substituted C₂-C₁₀ alkynyl. In some embodiments, R₂ is unsubstituted C₂-C₁₀ alkenyl. In some embodiments, R₂ is substituted C₂-C₁₀ alkenyl. In some embodiments, R₂ is carboxyl. In some embodiments, R₂ is unsubstituted carbohydrate. In some embodiments, R₂ is substituted carbohydrate. In some embodiments, R₂ is unsubstituted ester. In some embodiments, R₂ is substituted ester. In some embodiments, R₂ is unsubstituted acyloxy. In some embodiments, R₂ is substituted acyloxy. In some embodiments, R₂ is nitro. In some embodiments, R₂ is halogen. In some embodiments, R₂ is unsubstituted C₁-C₁₀ aliphatic acyl. In some embodiments, R₂ is substituted C₁-C₁₀ aliphatic acyl. In some embodiments, R₂ is unsubstituted C₆-C₁₀ aromatic acyl. In some embodiments, R₂ is substituted C₆-C₁₀ aromatic acyl. In some embodiments, R₂ is unsubstituted C₆-C₁₀ aralkyl acyl. In some embodiments, R₂ is substituted C₆-C₁₀ aralkyl acyl. In some embodiments, R₂ is unsubstituted C₆-C₁₀ alkylaryl acyl. In some embodiments, R₂ is substituted C₆-C₁₀ alkylaryl acyl. In some embodiments, R₂ is unsubstituted alkoxy. In some embodiments, R₂ is substituted alkoxy. In some embodiments, R₂ is unsubstituted amine. In some embodiments, R₂ is substituted amine. In some embodiments, R₂ is unsubstituted aryl. In some embodiments, R₂ is substituted aryl. In some embodiments, R₂ is unsubstituted C₄-C₁₀heterocyclyl. In some embodiments, R₂ is substituted C₄-C₁₀heterocyclyl. In some embodiments, R₂ is unsubstituted heteroaryl. In some embodiments, R₂ is substituted heteroaryl. In some embodiments, R₂ is unsubstituted C₃-C₁₀cycloalkyl. In some embodiments, R₂ is substituted C₃-C₁₀cycloalkyl. In some embodiments, R₂ is —OPO₃WY. In some embodiments, R₂ is —OCH₂PO₄WY. In some embodiments, R₂ is —OCH₂PO₄Z. In some embodiments, R₂ is —OPO₃Z.

In some embodiments, R₅ is hydrogen. In some embodiments, R₅ is hydroxyl. In some embodiments, R₅ is carboxaldehyde. In some embodiments, R₅ is unsubstituted amine. In some embodiments, R₅ is substituted amine. In some embodiments, R₅ is unsubstituted C₁-C₁₀ alkyl. In some embodiments, R₅ is substituted C₁-C₁₀ alkyl. In some embodiments, R₅ is unsubstituted C₂-C₁₀ alkynyl. In some embodiments, R₅ is substituted C₂-C₁₀ alkynyl. In some embodiments, R₅ is unsubstituted C₂-C₁₀ alkenyl. In some embodiments, R₅ is substituted C₂-C₁₀ alkenyl. In some embodiments, R₅ is carboxyl. In some embodiments, R₅ is unsubstituted carbohydrate. In some embodiments, R₅ is substituted carbohydrate. In some embodiments, R₅ is unsubstituted ester. In some embodiments, R₅ is substituted ester. In some embodiments, R₅ is unsubstituted acyloxy. In some embodiments, R₅ is substituted acyloxy. In some embodiments, R₅ is nitro. In some embodiments, R₅ is halogen. In some embodiments, R₅ is unsubstituted C₁-C₁₀ aliphatic acyl. In some embodiments, R₅ is substituted C₁-C₁₀ aliphatic acyl. In some embodiments, R₅ is unsubstituted C₆-C₁₀ aromatic acyl. In some embodiments, R₅ is substituted C₆-C₁₀ aromatic acyl. In some embodiments, R₅ is unsubstituted C₆-C₁₀ aralkyl acyl. In some embodiments, R₅ is substituted C₆-C₁₀ aralkyl acyl. In some embodiments, R₅ is unsubstituted C₆-C₁₀ alkylaryl acyl. In some embodiments, R₅ is substituted C₆-C₁₀ alkylaryl acyl. In some embodiments, R₅ is unsubstituted alkoxy. In some embodiments, R₅ is substituted alkoxy. In some embodiments, R₅ is unsubstituted aryl. In some embodiments, R₅ is substituted aryl. In some embodiments, R₅ is unsubstituted C₃-C₁₀heterocyclyl. In some embodiments, R₅ is substituted C₃-C₁₀ heterocyclyl. In some embodiments, R₅ is unsubstituted heteroaryl, In some embodiments, R₅ is unsubstituted C₃-C₁₀cycloalkyl. In some embodiments, R₅ is substituted C₃-C₁₀cycloalkyl. In some embodiments, R₅ is —OPO₃WY. In some embodiments, R₅ is —OCH₂PO₄WY. In some embodiments, R₅ is —OCH₂PO₄Z. In some embodiments, R₅ is —OPO₃Z.

In some embodiments, R₁₀ is hydrogen. In some embodiments, R₁₀ is hydroxyl. In some embodiments, R₁₀ is carboxaldehyde. In some embodiments, R₁₀ is unsubstituted amine. In some embodiments, R₁₀ is substituted amine. In some embodiments, R₁₀ is unsubstituted C₁-C₁₀ alkyl. In some embodiments, R₁₀ is substituted C₁-C₁₀ alkyl. In some embodiments, R₁₀ is unsubstituted C₂-C₁₀ alkynyl. In some embodiments, R₁₀ is substituted C₂-C₁₀ alkynyl. In some embodiments, R₁₀ is unsubstituted C₂-C₁₀ alkenyl. In some embodiments, R₁₀ is substituted C₂-C₁₀ alkenyl. In some embodiments, R₁₀ is carboxyl. In some embodiments, R₁₀ is unsubstituted carbohydrate. In some embodiments, R₁₀ is substituted carbohydrate. In some embodiments, R₁₀ is unsubstituted ester. In some embodiments, R₁₀ is substituted ester. In some embodiments, R₁₀ is unsubstituted acyloxy. In some embodiments, R₁₀ is substituted acyloxy. In some embodiments, R₁₀ is nitro. In some embodiments, R₁₀ is halogen. In some embodiments, R₁₀ is unsubstituted C₁-C₁₀ aliphatic acyl. In some embodiments, R₁₀ is substituted C₁-C₁₀ aliphatic acyl. In some embodiments, R₁₀ is unsubstituted C₆-C₁₀ aromatic acyl. In some embodiments, R₁₀ is substituted C₆-C₁₀ aromatic acyl. In some embodiments, R₁₀ is unsubstituted C₆-C₁₀ aralkyl acyl. In some embodiments, R₁₀ is substituted C₆-C₁₀ aralkyl acyl. In some embodiments, R₁₀ is unsubstituted C₆-C₁₀ alkylaryl acyl. In some embodiments, R₁₀ is substituted C₆-C₁₀ alkylaryl acyl. In some embodiments, R₁₀ is unsubstituted alkoxy. In some embodiments, R₁₀ is substituted alkoxy. In some embodiments, R₁₀ is unsubstituted aryl. In some embodiments, R₁₀ is substituted aryl. In some embodiments, R₁₀ is unsubstituted C₃-C₁₀heterocyclyl. In some embodiments, R₁₀ is substituted C₃-C₁₀heterocyclyl. In some embodiments, R₁₀ is unsubstituted heteroaryl, In some embodiments, R₁₀ is unsubstituted C₃-C₁₀cycloalkyl. In some embodiments, R₁₀ is substituted C₃-C₁₀cycloalkyl. In some embodiments, R₁₀ is —OPO₃WY. In some embodiments, R₁₀ is —OCH₂PO₄WY. In some embodiments, R₁₀ is —OCH₂PO₄Z. In some embodiments, R₁₀ is —OPO₃Z.

In some embodiments, R₁₁ is hydrogen. In some embodiments, R₁₁ is hydroxyl. In some embodiments, R₁₁ is carboxaldehyde. In some embodiments, R₁₁ is unsubstituted amine. In some embodiments, R₁₁ is substituted amine. In some embodiments, R₁₁ is unsubstituted C₁-C₁₀ alkyl. In some embodiments, R₁₁ is substituted C₁-C₁₀ alkyl. In some embodiments, R₁₁ is unsubstituted C₂-C₁₀ alkynyl. In some embodiments, R₁₁ is substituted C₂-C₁₀ alkynyl. In some embodiments, R₁₁ is unsubstituted C₂-C₁₀ alkenyl. In some embodiments, R₁₁ is substituted C₂-C₁₀ alkenyl. In some embodiments, R₁₁ is carboxyl. In some embodiments, R₁₁ is unsubstituted carbohydrate. In some embodiments, R₁₁ is substituted carbohydrate. In some embodiments, R₁₁ is unsubstituted ester. In some embodiments, R₁₁ is substituted ester. In some embodiments, R₁₁ is unsubstituted acyloxy. In some embodiments, R₁₁ is substituted acyloxy. In some embodiments, R₁₁ is nitro. In some embodiments, R₁₁ is halogen. In some embodiments, R₁₁ is unsubstituted C₁-C₁₀ aliphatic acyl. In some embodiments, R₁₁ is substituted C₁-C₁₀ aliphatic acyl. In some embodiments, R₁₁ is unsubstituted C₆-C₁₀ aromatic acyl. In some embodiments, R₁₁ is substituted C₆-C₁₀ aromatic acyl. In some embodiments, R₁₁ is unsubstituted C₆-C₁₀ aralkyl acyl. In some embodiments, R₁₁ is substituted C₆-C₁₀ aralkyl acyl. In some embodiments, R₁₁ is unsubstituted C₆-C₁₀ alkylaryl acyl. In some embodiments, R₁₁ is substituted C₆-C₁₀ alkylaryl acyl. In some embodiments, R₁₁ is unsubstituted alkoxy. In some embodiments, R₁₁ is substituted alkoxy. In some embodiments, R₁₁ is unsubstituted aryl. In some embodiments, R₁₁ is substituted aryl. In some embodiments, R₁₁ is unsubstituted C₃-C₁₀heterocyclyl. In some embodiments, R₁₁ is substituted C₃-C₁₀heterocyclyl. In some embodiments, R₁₁ is unsubstituted heteroaryl, In some embodiments, R₁₁ is unsubstituted C₃-C₁₀cycloalkyl. In some embodiments, R₁₁ is substituted C₃-C₁₀cycloalkyl. In some embodiments, R₁₁ is —OPO₃WY. In some embodiments, R₁₁ is —OCH₂PO₄WY. In some embodiments, R₁₁ is —OCH₂PO₄Z. In some embodiments, R₁₁ is —OPO₃Z.

In some embodiments, R₁₆ is hydrogen. In some embodiments, R₁₆ is unsubstituted C₁-C₁₀ alkyl. In some embodiments, R₁₆ is substituted C₁-C₁₀ alkyl. In some embodiments, R₁₆ is unsubstituted C₂-C₁₀ alkynyl. In some embodiments, R₁₆ is substituted C₂-C₁₀ alkynyl. In some embodiments, R₁₆ is unsubstituted C₂-C₁₀ alkenyl. In some embodiments, R₁₆ is substituted C₂-C₁₀ alkenyl. In some embodiments, R₁₆ is unsubstituted carbohydrate 1. In some embodiments, R₁₆ is substituted carbohydrate. In some embodiments, R₁₆ is unsubstituted C₁-C₁₀ aliphatic acyl. In some embodiments, R₁₆ is substituted C₁-C₁₀ aliphatic acyl. In some embodiments, R₁₆ is unsubstituted C₆-C₁₀ aromatic acyl. In some embodiments, R₁₆ is substituted C₆-C₁₀ aromatic acyl. In some embodiments, R₁₆ is unsubstituted C₆-C₁₀ aralkyl acyl. In some embodiments, R₁₆ is substituted C₆-C₁₀ aralkyl acyl. In some embodiments, R₁₆ is unsubstituted C₆-C₁₀ alkylaryl acyl. In some embodiments, R₁₆ is substituted C₆-C₁₀ alkylaryl acyl. In some embodiments, R₁₆ is unsubstituted aryl. In some embodiments, R₁₆ is substituted aryl. In some embodiments, R₁₆ is unsubstituted C₃-C₁₀heterocyclyl. In some embodiments, R₁₆ is substituted C₃-C₁₀ heterocyclyl. In some embodiments, R₁₆ is unsubstituted heteroaryl. In some embodiments, R₁₆ is substituted heteroaryl. In some embodiments, R₁₆ is unsubstituted C₃-C₁₀cycloalkyl. In some embodiments, R₁₆ is substituted C₃-C₁₀cycloalkyl. In some embodiments, R₁₆ is —PO₃WY. In some embodiments, R₁₆ is —CH₂PO₄WY. In some embodiments, R₁₆ is —CH₂PO₄Z. In some embodiments, R₁₆ is —PO₃Z.

In some embodiments, R₁₇ is hydrogen. In some embodiments, R₁₇ is hydroxy. In some embodiments, R₁₇ is carboxaldehyde. In some embodiments, R₁₇ is unsubstituted amine. In some embodiments, R₁₇ is substituted amine. In some embodiments, R₁₇ is unsubstituted C₁-C₁₀ alkyl. In some embodiments, R₁₇ is unsubstituted C₂-C₁₀ alkynyl. In some embodiments, R₁₇ is substituted C₂-C₁₀ alkynyl. In some embodiments, R₁₇ is unsubstituted C₂-C₁₀ alkenyl. In some embodiments, R₁₇ is substituted C₂-C₁₀ alkenyl. In some embodiments, R₁₇ is carboxyl. In some embodiments, R₁₇ is unsubstituted carbohydrate. In some embodiments, R₁₇ is substituted carbohydrate. In some embodiments, R₁₇ is unsubstituted ester. In some embodiments, R₁₇ is substituted ester. In some embodiments, R₁₇ is unsubstituted acyloxy. In some embodiments, R₁₇ is substituted acyloxy. In some embodiments, R₁₇ is nitro. In some embodiments, R₁₇ is halogen. In some embodiments, R₁₇ is unsubstituted C₁-C₁₀ aliphatic acyl. In some embodiments, R₁₇ is substituted C₁-C₁₀ aliphatic acyl. In some embodiments, R₁₇ is unsubstituted C₆-C₁₀ aromatic acyl. In some embodiments, R₁₇ is substituted C₆-C₁₀ aromatic acyl. In some embodiments, R₁₇ is unsubstituted C₆-C₁₀ aralkyl acyl. In some embodiments, R₁₇ is substituted C₆-C₁₀ aralkyl acyl. In some embodiments, R₁₇ is unsubstituted C₆-C₁₀ alkylaryl acyl. In some embodiments, R₁₇ is substituted C₆-C₁₀ alkylaryl acyl. In some embodiments, R₁₇ is unsubstituted alkoxy. In some embodiments, R₁₇ is substituted alkoxy. In some embodiments, R₁₇ is unsubstituted aryl. In some embodiments, R₁₇ is substituted aryl. In some embodiments, R₁₇ is unsubstituted C₃-C₁₀heterocyclyl. In some embodiments, R₁₇ is substituted C₃-C₁₀heterocyclyl. In some embodiments, R₁₇ is unsubstituted heteroaryl. In some embodiments, R₁₇ is substituted heteroaryl. In some embodiments, R₁₇ is unsubstituted C₃-C₁₀cycloalkyl. In some embodiments, R₁₇ is substituted C₃-C₁₀cycloalkyl. In some embodiments, R₁₇ is —OPO₃WY. In some embodiments, R₁₇ is —OCH₂PO₄WY. In some embodiments, R₁₇ is —OCH₂PO₄Z. In some embodiments, R₁₇ is —OPO₃Z.

In some embodiments, R₁₈ is hydrogen. In some embodiments, R₁₈ is hydroxy. In some embodiments, R₁₈ is carboxaldehyde. In some embodiments, R₁₈ is unsubstituted amine. In some embodiments, R₁₈ is substituted amine. In some embodiments, R₁₈ is unsubstituted C₁-C₁₀ alkyl. In some embodiments, R₁₈ is unsubstituted C₂-C₁₀ alkynyl. In some embodiments, R₁₈ is substituted C₂-C₁₀ alkynyl. In some embodiments, R₁₈ is unsubstituted C₂-C₁₀ alkenyl. In some embodiments, R₁₈ is substituted C₂-C₁₀ alkenyl. In some embodiments, R₁₈ is carboxyl. In some embodiments, R₁₈ is unsubstituted carbohydrate. In some embodiments, R₁₈ is substituted carbohydrate. In some embodiments, R₁₈ is substituted carbohydrate. In some embodiments, R₁₈ is unsubstituted ester. In some embodiments, R₁₈ is substituted ester. In some embodiments, R₁₈ is unsubstituted acyloxy. In some embodiments, R₁₈ is substituted acyloxy. In some embodiments, R₁₈ is nitro. In some embodiments, R₁₈ is halogen. In some embodiments, R₁₈ is unsubstituted C₁-C₁₀ aliphatic acyl. In some embodiments, R₁₈ is substituted C₁-C₁₀ aliphatic acyl. In some embodiments, R₁₈ is unsubstituted C₆-C₁₀ aromatic acyl. In some embodiments, R₁₈ is substituted C₆-C₁₀ aromatic acyl. In some embodiments, R₁₈ is unsubstituted C₆-C₁₀ aralkyl acyl. In some embodiments, R₁₈ is substituted C₆-C₁₀ aralkyl acyl. In some embodiments, R₁₈ is unsubstituted C₆-C₁₀ alkylaryl acyl. In some embodiments, R₁₈ is substituted C₆-C₁₀ alkylaryl acyl. In some embodiments, R₁₈ is unsubstituted alkoxy. In some embodiments, R₁₈ is substituted alkoxy. In some embodiments, R₁₈ is unsubstituted aryl. In some embodiments, R₁₈ is substituted aryl. In some embodiments, R₁₈ is unsubstituted C₃-C₁₀heterocyclyl. In some embodiments, R₁₈ is substituted C₃-C₁₀heterocyclyl. In some embodiments, R₁₈ is unsubstituted heteroaryl. In some embodiments, R₁₈ is substituted heteroaryl. In some embodiments, R₁₈ is unsubstituted C₃-C₁₀cycloalkyl. In some embodiments, R₁₈ is substituted C₃-C₁₀cycloalkyl. In some embodiments, R₁₈ is —OPO₃WY. In some embodiments, R₁₈ is —OCH₂PO₄WY. In some embodiments, R₁₈ is —OCH₂PO₄Z. In some embodiments, R₁₈ is —OPO₃Z.

In some embodiments, R₁₉ is hydrogen. In some embodiments, R₁₉ is unsubstituted C₁-C₁₀ alkyl. In some embodiments, R₁₉ is substituted C₁-C₁₀ alkyl. In some embodiments, R₁₉ is unsubstituted C₂-C₁₀ alkynyl. In some embodiments, R₁₉ is substituted C₂-C₁₀ alkynyl. In some embodiments, R₁₉ is unsubstituted C₂-C₁₀ alkenyl. In some embodiments, R₁₉ is substituted C₂-C₁₀ alkenyl. In some embodiments, R₁₉ is unsubstituted carbohydrate. In some embodiments, R₁₉ is substituted carbohydrate. In some embodiments, R₁₉ is unsubstituted C₁-C₁₀ aliphatic acyl. In some embodiments, R₁₉ is substituted C₁-C₁₀ aliphatic acyl. In some embodiments, R₁₉ is unsubstituted C₆-C₁₀ aromatic acyl. In some embodiments, R₁₉ is substituted C₆-C₁₀ aromatic acyl. In some embodiments, R₁₉ is unsubstituted C₆-C₁₀ aralkyl acyl. In some embodiments, R₁₉ is substituted C₆-C₁₀ aralkyl acyl. In some embodiments, R₁₉ is unsubstituted C₆-C₁₀ alkylaryl acyl. In some embodiments, R₁₉ is substituted C₆-C₁₀ alkylaryl acyl. In some embodiments, R₁₉ is unsubstituted aryl. In some embodiments, R₁₉ is substituted aryl. In some embodiments, R₁₉ is unsubstituted C₃-C₁₀heterocyclyl. In some embodiments, R₁₉ is substituted C₃-C₁₀ heterocyclyl. In some embodiments, R₁₉ is unsubstituted heteroaryl. In some embodiments, R₁₉ is substituted heteroaryl. In some embodiments, R₁₉ is unsubstituted C₃-C₁₀cycloalkyl. In some embodiments, R₁₉ is substituted C₃-C₁₀cycloalkyl. In some embodiments, R₁₉ is —PO₃WY. In some embodiments, R₁₉ is —CH₂PO₄WY. In some embodiments, R₁₉ is —CH₂PO₄Z. In some embodiments, R₁₉ is —PO₃Z.

In some embodiments, R₂₁ is hydrogen. In some embodiments, R₂₁ is hydroxy. In some embodiments, R₂₁ is carboxaldehyde. In some embodiments, R₂₁ is unsubstituted amine. In some embodiments, R₂₁ is substituted amine. In some embodiments, R₂₁ is unsubstituted C₁-C₁₀ alkyl. In some embodiments, R₂₁ is unsubstituted C₂-C₁₀ alkynyl. In some embodiments, R₂₁ is substituted C₂-C₁₀ alkynyl. In some embodiments, R₂₁ is unsubstituted C₂-C₁₀ alkenyl. In some embodiments, R₂₁ is substituted C₂-C₁₀ alkenyl. In some embodiments, R₂₁ is carboxyl. In some embodiments, R₂₁ is unsubstituted carbohydrate. In some embodiments, R₂₁ is substituted carbohydrate. In some embodiments, R₂₁ is unsubstituted ester. In some embodiments, R₂₁ is substituted ester. In some embodiments, R₂₁ is unsubstituted acyloxy. In some embodiments, R₂₁ is substituted acyloxy. In some embodiments, R₂₁ is nitro. In some embodiments, R₂₁ is halogen. In some embodiments, R₂₁ is unsubstituted C₁-C₁₀ aliphatic acyl. In some embodiments, R₂₁ is substituted C₁-C₁₀ aliphatic acyl. In some embodiments, R₂₁ is unsubstituted C₆-C₁₀ aromatic acyl. In some embodiments, R₂₁ is substituted C₆-C₁₀ aromatic acyl. In some embodiments, R₂₁ is unsubstituted C₆-C₁₀ aralkyl acyl. In some embodiments, R₂₁ is substituted C₆-C₁₀ aralkyl acyl. In some embodiments, R₂₁ is unsubstituted C₆-C₁₀ alkylaryl acyl. In some embodiments, R₂₁ is substituted C₆-C₁₀ alkylaryl acyl. In some embodiments, R₂₁ is unsubstituted alkoxy. In some embodiments, R₂₁ is substituted alkoxy. In some embodiments, R₂₁ is unsubstituted aryl. In some embodiments, R₂₁ is substituted aryl. In some embodiments, R₂₁ is unsubstituted C₃-C₁₀heterocyclyl. In some embodiments, R₂₁ is substituted C₃-C₁₀heterocyclyl. In some embodiments, R₂₁ is unsubstituted heteroaryl. In some embodiments, R₂₁ is substituted heteroaryl. In some embodiments, R₂₁ is unsubstituted C₃-C₁₀cycloalkyl. In some embodiments, R₂₁ is substituted C₃-C₁₀cycloalkyl. In some embodiments, R₂₁ is —OPO₃WY. In some embodiments, R₂₁ is —OCH₂PO₄WY. In some embodiments, R₂₁ is —OCH₂PO₄Z. In some embodiments, R₂₁ is —OPO₃Z.

In some embodiments, s is an integer of 0. In some embodiments, s is an integer of 1. In some embodiments, s is an integer of 2. In some embodiments, s is an integer of 3.

In some embodiments, n is an integer of 0. In some embodiments, n is an integer of 1. In some embodiments, n is an integer of 2. In some embodiments, n is an integer of 3. In some embodiments, n is an integer of 4.

In various embodiments, W is hydrogen. In various embodiments, W is unsubstituted methyl. In various embodiments, W is substituted methyl. In various embodiments, W is unsubstituted ethyl. In various embodiments, W is substituted ethyl. In various embodiments, W is unsubstituted alkyl. In various embodiments, W is substituted alkyl. In various embodiments, W is unsubstituted carbohydrate. In various embodiments, W is substituted carbohydrate. In various embodiments, W is potassium. In various embodiments, W is sodium. In various embodiments, W is lithium.

In various embodiments, Y is hydrogen. In various embodiments, Y is unsubstituted methyl. In various embodiments, Y is substituted methyl. In various embodiments, Y is unsubstituted ethyl. In various embodiments, Y is substituted ethyl. In various embodiments, Y is unsubstituted alkyl. In various embodiments, Y is substituted alkyl. In various embodiments, Y is unsubstituted carbohydrate. In various embodiments, Y is substituted carbohydrate. In various embodiments, Y is potassium. In various embodiments, Y is sodium. In various embodiments, Y is lithium.

In various embodiments, Z is calcium. In various embodiments, Z is magnesium. In various embodiments, Z is iron.

In some embodiments of the invention, the pyrone analog of Formula IV is of Formula XXIV or Formula XXV or a pharmaceutically/veterinarily acceptable salt thereof:

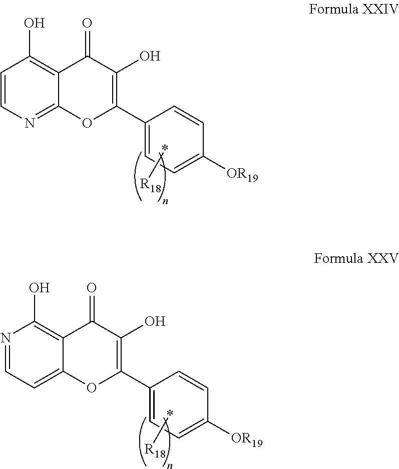

wherein each instance of R₁₈ is independently hydrogen, hydroxyl, carboxaldehyde, amine, C₁-C₁₀ alkyl, C₂-C₁₀ alkynyl, C₂-C₁₀ alkenyl, carboxyl, carbohydrate, ester, acyloxy, nitro, halogen, C₁-C₁₀ aliphatic acyl, C₆-C₁₀ aromatic acyl, C₆-C₁₀ aralkyl acyl, C₆-C₁₀ alkylaryl acyl, alkoxy, alkyl, phosphate, aryl, heteroaryl, C₃-C₁₀ heterocyclic, C₃-C₁₀cycloalkyl, —OPO₃WY, —OCH₂PO₄WY, —OCH₂PO₄Z or —OPO₃Z;

R₁₉ is hydrogen, C₁-C₁₀ alkyl, C₂-C₁₀ alkynyl, C₂-C₁₀ alkenyl, carbohydrate, C₁-C₁₀ aliphatic acyl, C₆-C₁₀ aromatic acyl, C₆-C₁₀ aralkyl acyl, C₆-C₁₀ alkylaryl acyl, aryl, C₃-C₁₀heterocyclyl, heteroaryl, optionally substituted C₃-C₁₀cycloalkyl, —PO₃WY, —CH₂PO₄WY, —CH₂PO₄Z or —PO₃Z;

n is an integer of 0, 1, 2, 3, or 4; and

W and Y are independently hydrogen, methyl, ethyl, alkyl, carbohydrate, or a cation, and Z is a multivalent cation.

In some embodiments of the invention, R₁₉ is —PO₃WY, —CH₂PO₄WY, —CH₂PO₄Z or —PO₃Z, or at least one of r R₁₈ is —OPO₃WY, —OCH₂PO₄WY, —OCH₂PO₄Z or —OPO₃Z. In some embodiments, R₁₉ is —PO₃WY, —CH₂PO₄WY, —CH₂PO₄Z or —PO₃Z and at least one of R₁₈ is —OPO₃WY, —OCH₂PO₄WY, —OCH₂PO₄Z or —OPO₃Z.

In some embodiments, R₁₈ is hydrogen. In some embodiments, R₁₈ is hydroxy. In some embodiments, R₁₈ is carboxaldehyde. In some embodiments, R₁₈ is unsubstituted amine. In some embodiments, R₁₈ is substituted amine. In some embodiments, R₁₈ is unsubstituted C₁-C₁₀ alkyl. In some embodiments, R₁₈ is unsubstituted C₂-C₁₀ alkynyl. In some embodiments, R₁₈ is substituted C₂-C₁₀ alkynyl. In some embodiments, R₁₈ is unsubstituted C₂-C₁₀ alkenyl. In some embodiments, R₁₈ is substituted C₂-C₁₀ alkenyl. In some embodiments, R₁₈ is carboxyl. In some embodiments, R₁₈ is unsubstituted carbohydrate. In some embodiments, R₁₈ is substituted carbohydrate. In some embodiments, R₁₈ is substituted carbohydrate. In some embodiments, R₁₈ is unsubstituted ester. In some embodiments, R₁₈ is substituted ester. In some embodiments, R₁₈ is unsubstituted acyloxy. In some embodiments, R₁₈ is substituted acyloxy. In some embodiments, R₁₈ is nitro. In some embodiments, R₁₈ is halogen. In some embodiments, R₁₈ is unsubstituted C₁-C₁₀ aliphatic acyl. In some embodiments, R₁₈ is substituted C₁-C₁₀ aliphatic acyl. In some embodiments, R₁₈ is unsubstituted C₆-C₁₀ aromatic acyl. In some embodiments, R₁₈ is substituted C₆-C₁₀ aromatic acyl. In some embodiments, R₁₈ is unsubstituted C₆-C₁₀ aralkyl acyl. In some embodiments, R₁₈ is substituted C₆-C₁₀ aralkyl acyl. In some embodiments, R₁₈ is unsubstituted C₆-C₁₀ alkylaryl acyl. In some embodiments, R₁₈ is substituted C₆-C₁₀ alkylaryl acyl. In some embodiments, R₁₈ is unsubstituted alkoxy. In some embodiments, R₁₈ is substituted alkoxy. In some embodiments, R₁₈ is unsubstituted aryl. In some embodiments, R₁₈ is substituted aryl. In some embodiments, R₁₈ is unsubstituted C₃-C₁₀heterocyclyl. In some embodiments, R₁₈ is substituted C₃-C₁₀heterocyclyl. In some embodiments, R₁₈ is unsubstituted heteroaryl. In some embodiments, R₁₈ is substituted heteroaryl. In some embodiments, R₁₈ is unsubstituted C₃-C₁₀cycloalkyl. In some embodiments, R₁₈ is substituted C₃-C₁₀cycloalkyl. In some embodiments, R₁₈ is —OPO₃WY. In some embodiments, R₁₈ is —OCH₂PO₄WY. In some embodiments, R₁₈ is —OCH₂PO₄Z. In some embodiments, R₁₈ is —OPO₃Z.

In some embodiments, R₁₉ is hydrogen. In some embodiments, R₁₉ is unsubstituted C₁-C₁₀ alkyl. In some embodiments, R₁₉ is substituted C₁-C₁₀ alkyl. In some embodiments, R₁₉ is unsubstituted C₂-C₁₀ alkynyl. In some embodiments, R₁₉ is substituted C₂-C₁₀ alkynyl. In some embodiments, R₁₉ is unsubstituted C₂-C₁₀ alkenyl. In some embodiments, R₁₉ is substituted C₂-C₁₀ alkenyl. In some embodiments, R₁₉ is unsubstituted carbohydrate. In some embodiments, R₁₉ is substituted carbohydrate. In some embodiments, R₁₉ is unsubstituted C₁-C₁₀ aliphatic acyl. In some embodiments, R₁₉ is substituted C₁-C₁₀ aliphatic acyl. In some embodiments, R₁₉ is unsubstituted C₆-C₁₀ aromatic acyl. In some embodiments, R₁₉ is substituted C₆-C₁₀ aromatic acyl. In some embodiments, R₁₉ is unsubstituted C₆-C₁₀ aralkyl acyl. In some embodiments, R₁₉ is substituted C₆-C₁₀ aralkyl acyl. In some embodiments, R₁₉ is unsubstituted C₆-C₁₀ alkylaryl acyl. In some embodiments, R₁₉ is substituted C₆-C₁₀ alkylaryl acyl. In some embodiments, R₁₉ is unsubstituted aryl. In some embodiments, R₁₉ is substituted aryl. In some embodiments, R₁₉ is unsubstituted C₃-C₁₀heterocyclyl. In some embodiments, R₁₉ is substituted C₃-C₁₀ heterocyclyl. In some embodiments, R₁₉ is unsubstituted heteroaryl. In some embodiments, R₁₉ is substituted heteroaryl. In some embodiments, R₁₉ is unsubstituted C₃-C₁₀cycloalkyl. In some embodiments, R₁₉ is substituted C₃-C₁₀cycloalkyl. In some embodiments, R₁₉ is —PO₃WY. In some embodiments, R₁₉ is —CH₂PO₄WY. In some embodiments, R₁₉ is —CH₂PO₄Z. In some embodiments, R₁₉ is —PO₃Z.

In some embodiments, n is an integer of 0. In some embodiments, n is an integer of 1. In some embodiments, n is an integer of 2. In some embodiments, n is an integer of 3. In some embodiments, n is an integer of 4.

In various embodiments, W and Y are independently potassium, sodium, or lithium.

In various embodiments, Z is calcium, magnesium or iron.

In some embodiments of the invention, the pyrone analog of Formula IV is of Formula XXVI or Formula XXVII or a pharmaceutically/veterinarily acceptable salt thereof:

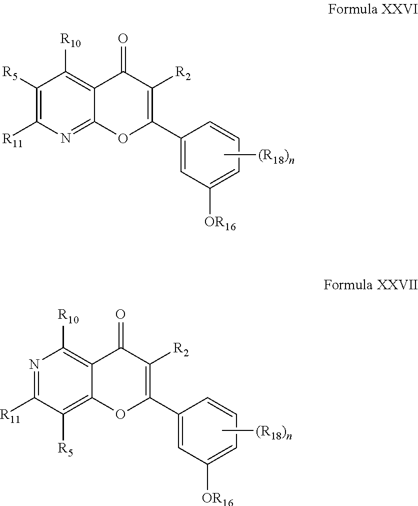

wherein R₂, R₅, W, Y, and Z are as defined for Formula II and R₁₀ and R₁₁ are as defined for Formula IV;

R₁₆ is hydrogen, —PO₃WY, —CH₂PO₄WY, —CH₂PO₄Z or —PO₃Z;

wherein each instance of R₁₈ is independently hydrogen, hydroxyl, carboxaldehyde, amine, C₁-C₁₀ alkyl, C₂-C₁₀ alkynyl, C₂-C₁₀ alkenyl, carboxyl, carbohydrate, ester, acyloxy, nitro, halogen, C₁-C₁₀ aliphatic acyl, C₆-C₁₀ aromatic acyl, C₆-C₁₀ aralkyl acyl, C₆-C₁₀ alkylaryl acyl, alkoxy, alkyl, phosphate, aryl, heteroaryl, C₃-C₁₀ heterocyclic, C₃-C₁₀cycloalkyl, —OPO₃WY, —OCH₂PO₄WY, —OCH₂PO₄Z or —OPO₃Z; and

n is an integer of 0, 1, 2, 3, or 4.

In some embodiments of the invention, R₁₆ is —PO₃WY, —CH₂PO₄WY, —CH₂PO₄Z or —PO₃Z, or at least one of R₁₈ is —OPO₃WY, —OCH₂PO₄WY, —OCH₂PO₄Z or —OPO₃Z. In some embodiments, R₁₆ is —PO₃WY, —CH₂PO₄WY, —CH₂PO₄Z or —PO₃Z and at least one of R₁₈ is —OPO₃WY, —OCH₂PO₄WY, —OCH₂PO₄Z or —OPO₃Z.

In some embodiments of the invention, the pyrone analog of Formula IV is of Formula XXVIII or a pharmaceutically/veterinarily acceptable salt thereof:

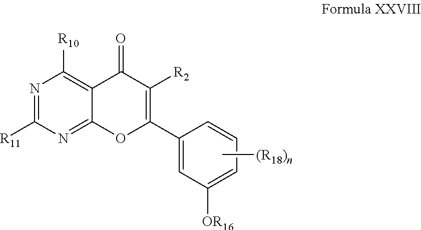

wherein R₂, W, Y, and Z are as defined for Formula II and R₁₀ and R₁₁ are as defined for Formula IV;

R₁₆ is hydrogen, —PO₃WY, —CH₂PO₄WY, —CH₂PO₄Z or —PO₃Z;

wherein each instance of R₁₈ is independently hydrogen, hydroxyl, carboxaldehyde, amine, C₁-C₁₀ alkyl, C₂-C₁₀ alkynyl, C₂-C₁₀ alkenyl, carboxyl, carbohydrate, ester, acyloxy, nitro, halogen, C₁-C₁₀ aliphatic acyl, C₆-C₁₀ aromatic acyl, C₆-C₁₀ aralkyl acyl, C₆-C₁₀ alkylaryl acyl, alkoxy, alkyl, phosphate, aryl, heteroaryl, C₃-C₁₀ heterocyclic, C₃-C₁₀cycloalkyl, —OPO₃WY, —OCH₂PO₄WY, —OCH₂PO₄Z or —OPO₃Z; and

n is an integer of 0, 1, 2, 3, or 4.

In some embodiments of the invention, R₁₆ is —PO₃WY, —CH₂PO₄WY, —CH₂PO₄Z or —PO₃Z, or at least one of R₁₈ is —OPO₃WY, —OCH₂PO₄WY, —OCH₂PO₄Z or —OPO₃Z. In some embodiments, R₁₆ is —PO₃WY, —CH₂PO₄WY, —CH₂PO₄Z or —PO₃Z and at least one of R₁₈ is —OPO₃WY, —OCH₂PO₄WY, —OCH₂PO₄Z or —OPO₃Z.

In some embodiments of the invention, the pyrone analog of Formula II is of Formula V or a pharmaceutically/veterinarily acceptable salt thereof:

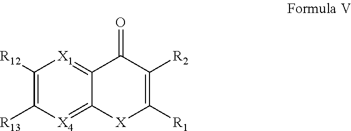

wherein X is O, S, or NR′, wherein R′ is hydrogen, C₁-C₁₀ alkyl, C₂-C₁₀ alkynyl, C₂-C₁₀ alkenyl, C₁-C₁₀ aliphatic acyl, C₆-C₁₀ aromatic acyl, C₆-C₁₀ aralkyl acyl, C₆-C₁₀ alkylaryl acyl, aryl, C₃-C₁₀ heterocyclyl, heteroaryl, or C₃-C₁₀cycloalkyl;

R₁, and R₂ are independently hydrogen, hydroxyl, C₁-C₁₀ alkyl, C₂-C₁₀ alkynyl, C₂-C₁₀ alkenyl, carboxyl, carbohydrate, ester, acyloxy, nitro, halogen, C₁-C₁₀ aliphatic acyl, C₆-C₁₀ aromatic acyl, C₆-C₁₀ aralkyl acyl, C₆-C₁₀ alkylaryl acyl, alkoxy, amine, aryl, C₄-C₁₀heterocyclyl, heteroaryl, C₃-C₁₀cycloalkyl, —OPO₃WY, —OCH₂PO₄WY, —OCH₂PO₄Z or —OPO₃Z;

X₁ and X₄ are independently CR₅, O, S, or N;

each instance of R₅ is independently hydrogen, hydroxyl, carboxaldehyde, amino, C₁-C₁₀ alkyl, C₂-C₁₀ alkynyl, C₂-C₁₀ alkenyl, carboxyl, carbohydrate, ester, acyloxy, nitro, halogen, C₁-C₁₀ aliphatic acyl, C₆-C₁₀ aromatic acyl, C₆-C₁₀ aralkyl acyl, C₆-C₁₀alkylaryl acyl, alkoxy, amine, aryl, C₃-C₁₀heterocyclyl, heteroaryl, C₃-C₁₀cycloalkyl, —OPO₃WY, —OCH₂PO₄WY, —OCH₂PO₄Z or —OPO₃Z;

R₁₂ and R₁₃ are independently hydrogen, hydroxyl, carboxaldehyde, amino, C₁-C₁₀ alkyl, C₂-C₁₀ alkynyl, C₂-C₁₀ alkenyl, carboxyl, carbohydrate, ester, acyloxy, nitro, halogen, C₁-C₁₀ aliphatic acyl, C₆-C₁₀ aromatic acyl, C₆-C₁₀ aralkyl acyl, C₆-C₁₀ alkylaryl acyl, alkoxy, amine, aryl, C₃-C₁₀ heterocyclyl, heteroaryl, C₃-C₁₀cycloalkyl, —OPO₃WY, —OCH₂PO₄WY, —OCH₂PO₄Z or —OPO₃Z;

W and Y are independently hydrogen, methyl, ethyl, alkyl, carbohydrate, or a cation, and Z is a multivalent cation; and

wherein the compound of Formula V is not the compound of Formula A:

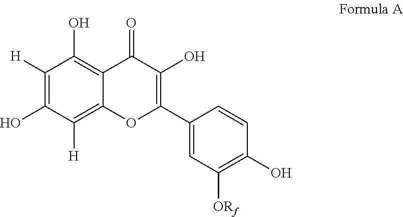

wherein R_{f} is hydrogen.

In some embodiments, the compound of Formula V is not the compound of Formula A wherein R_{f} is PO₃K₂.

In some embodiments, X is O.

In other embodiments, X is S.

In yet other embodiments, X is NR′.

In some embodiments, R′ is hydrogen. In some embodiments, R′ is unsubstituted C₁-C₁₀ alkyl. In some embodiments, R′ is substituted C₁-C₁₀ alkyl. In some embodiments, R′ is unsubstituted C₂-C₁₀ alkynyl. In some embodiments, R′ is substituted C₂-C₁₀ alkynyl. In some embodiments, R′ is unsubstituted C₂-C₁₀ alkenyl. In some embodiments, R′ is substituted C₂-C₁₀ alkenyl. In some embodiments, R′ is unsubstituted C₂-C₁₀ alkenyl. In some embodiments, R′ is substituted C₂-C₁₀ alkenyl. In some embodiments, R′ is unsubstituted C₁-C₁₀ aliphatic acyl. In some embodiments, R′ is substituted C₁-C₁₀ aliphatic acyl. In some embodiments, R′ is unsubstituted C₆-C₁₀ aromatic acyl. In some embodiments, R′ is substituted C₆-C₁₀ aromatic acyl. In some embodiments, R′ is unsubstituted C₆-C₁₀ aralkyl acyl. In some embodiments, R′ is substituted C₆-C₁₀ aralkyl acyl. In some embodiments, R′ is unsubstituted C₆-C₁₀ alkylaryl acyl. In some embodiments, R′ is substituted C₆-C₁₀ alkylaryl acyl. In some embodiments, R′ is unsubstituted aryl. In some embodiments, R′ is substituted aryl. In some embodiments, R′ is unsubstituted C₃-C₁₀ heterocyclyl. In some embodiments, R′ is substituted C₃-C₁₀ heterocyclyl. In some embodiments, R′ is unsubstituted heteroaryl. In some embodiments, R′ is substituted heteroaryl. In some embodiments, R′ is unsubstituted C₃-C₁₀cycloalkyl. In some embodiments, R′ is substituted C₃-C₁₀cycloalkyl.

In some embodiments, X₁ is CR₅.

In other embodiments, X₁ is O.

In yet other embodiments, X₁ is S.

In further embodiments, X₁ is N.

In other embodiments, X₄ is CR₅.

In some embodiments, X₄ is O.

In yet other embodiments, X₄ is S.

In some embodiments, X₄ is N.

In some embodiments, at least one of X₁ or X₄ is N.

In some embodiments, R₁ is hydrogen. In some embodiments, R₁ is hydroxyl. In some embodiments, R₁ is optionally substituted C₁-C₁₀ alkyl. In some embodiments, R₁ is unsubstituted C₁-C₁₀ alkyl. In some embodiments, R₁ is substituted C₁-C₁₀ alkyl. In some embodiments, R₁ is unsubstituted C₁-C₁₀ alkyl. In some other embodiments, R₁ is substituted C₁-C₁₀ alkyl. In some embodiments, R₁ is unsubstituted C₂-C₁₀ alkynyl. In some embodiments, R₁ is substituted C₂-C₁₀ alkynyl. In some embodiments, R₁ is unsubstituted C₂-C₁₀ alkenyl. In some embodiments, R₁ is substituted C₂-C₁₀ alkenyl. In some embodiments, R₁ is carboxyl. In some embodiments, R₁ is unsubstituted carbohydrate. In some embodiments, R₁ is substituted carbohydrate. In some embodiments, R₁ is unsubstituted ester. In some embodiments, R₁ is substituted ester. In some embodiments, R₁ is unsubstituted acyloxy. In some embodiments, R₁ is substituted acyloxy. In some embodiments, R₁ is nitro. In some embodiments, R₁ is halogen. In some embodiments, R₁ is unsubstituted C₁-C₁₀ aliphatic acyl. In some embodiments, R₁ is substituted C₁-C₁₀ aliphatic acyl. In some embodiments, R₁ is unsubstituted C₆-C₁₀ aromatic acyl. In some embodiments, R₁ is substituted C₆-C₁₀ aromatic acyl. In some embodiments, R₁ is unsubstituted C₆-C₁₀ aralkyl acyl. In some embodiments, R₁ is substituted C₆-C₁₀ aralkyl acyl. In some embodiments, R₁ is unsubstituted C₆-C₁₀ alkylaryl acyl. In some embodiments, R₁ is substituted C₆-C₁₀ alkylaryl acyl. In some embodiments, R₁ is unsubstituted alkoxy. In some embodiments, R₁ is substituted alkoxy. In some embodiments, R₁ is unsubstituted amine. In some embodiments, R₁ is substituted amine. In some embodiments, R₁ is unsubstituted aryl. In some embodiments, R₁ is substituted aryl. In some embodiments, R₁ is unsubstituted C₄-C₁₀heterocyclyl. In some embodiments, R₁ is substituted C₄-C₁₀heterocyclyl. In some embodiments, R₁ is unsubstituted heteroaryl. In some embodiments, R₁ is substituted heteroaryl. In some embodiments, R₁ is unsubstituted C₃-C₁₀cycloalkyl. In some embodiments, R₁ is substituted C₃-C₁₀cycloalkyl. In some embodiments, R₁ is —OPO₃WY. In some embodiments, R₁ is —OCH₂PO₄WY. In some embodiments, R₁ is —OCH₂PO₄Z. In some embodiments, R₁ is —OPO₃Z.

In some embodiments, when R₁ is aryl, it is monocyclic. In some embodiments, when R₁ is aryl, it is bicyclic. In some embodiments, when R₁ is heteroaryl, it is monocyclic. In some embodiments, when R₁ is heteroaryl, it is bicyclic.

In various embodiments, R₁ is one of the following formulae:

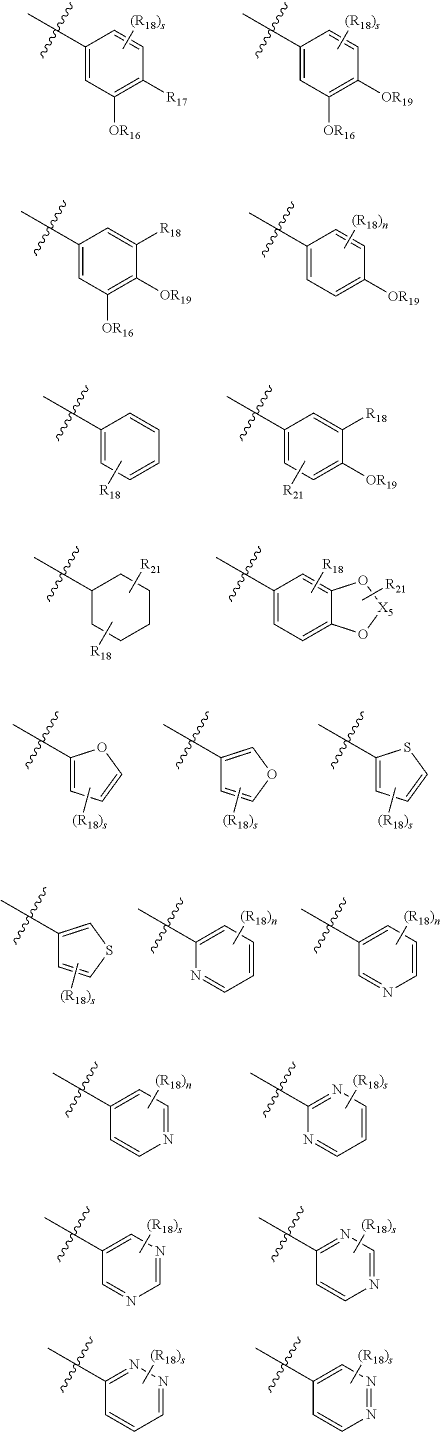

wherein R₁₆ is hydrogen, C₁-C₁₀ alkyl, C₂-C₁₀ alkynyl, C₂-C₁₀ alkenyl, carbohydrate, C₁-C₁₀ aliphatic acyl, C₆-C₁₀ aromatic acyl, C₆-C₁₀ aralkyl acyl, C₆-C₁₀ alkylaryl acyl, aryl, C₃-C₁₀ heterocyclyl, heteroaryl, C₃-C₁₀cycloalkyl, —PO₃WY, —CH₂PO₄WY, —CH₂PO₄Z or —PO₃Z;

R₁₇ is hydrogen, hydroxy, carboxaldehyde, amine, C₁-C₁₀ alkyl, C₂-C₁₀ alkynyl, C₂-C₁₀ alkenyl, carboxyl, carbohydrate, ester, acyloxy, nitro, halogen, C₁-C₁₀ aliphatic acyl, C₆-C₁₀ aromatic acyl, C₆-C₁₀ aralkyl acyl, C₆-C₁₀ alkylaryl acyl, alkoxy, aryl, C₃-C₁₀ heterocyclyl, heteroaryl, or C₃-C₁₀cycloalkyl, —OPO₃WY, —OCH₂PO₄WY, —OCH₂PO₄Z or —OPO₃Z;

each instance of R₁₈ and R₂₁ is independently hydrogen, hydroxyl, carboxaldehyde, amine, C₁-C₁₀ alkyl, C₂-C₁₀ alkynyl, C₂-C₁₀ alkenyl, carboxyl, carbohydrate, ester, acyloxy, nitro, halogen, C₁-C₁₀ aliphatic acyl, C₆-C₁₀ aromatic acyl, C₆-C₁₀ aralkyl acyl, C₆-C₁₀ alkylaryl acyl, alkoxy, alkyl, phosphate, aryl, heteroaryl, C₃-C₁₀ heterocyclic, C₃-C₁₀cycloalkyl, —OPO₃WY, —OCH₂PO₄WY, —OCH₂PO₄Z or —OPO₃Z;

R₁₉ is hydrogen, C₁-C₁₀ alkyl, C₂-C₁₀ alkynyl, C₂-C₁₀ alkenyl, carbohydrate, C₁-C₁₀ aliphatic acyl, C₆-C₁₀ aromatic acyl, C₆-C₁₀ aralkyl acyl, C₆-C₁₀ alkylaryl acyl, aryl, C₃-C₁₀heterocyclyl, heteroaryl, optionally substituted C₃-C₁₀cycloalkyl, —PO₃WY, —CH₂PO₄WY, —CH₂PO₄Z or —PO₃Z;

s is an integer of 0, 1, 2, or 3; and

n is an integer of 0, 1, 2, 3, or 4.

In some embodiments of the invention, at least one of R₁₆ and R₁₉ is —PO₃WY, —CH₂PO₄WY, —CH₂PO₄Z or —PO₃Z, or at least one of R₁₇ or R₁₈ is —OPO₃WY, —OCH₂PO₄WY, —OCH₂PO₄Z or —OPO₃Z. In some embodiments, at least one of R₁₆ and R₁₉ is —PO₃WY, —CH₂PO₄WY, —CH₂PO₄Z or —PO₃Z and at least one of R₁₇ or R₁₈ is —OPO₃WY, —OCH₂PO₄WY, —OCH₂PO₄Z or —OPO₃Z.

In some embodiments, R₂ is hydrogen. In some embodiments, R₂ is hydroxyl. In some embodiments, R₂ is optionally substituted C₁-C₁₀ alkyl. In some embodiments, R₂ is unsubstituted C₁-C₁₀ alkyl. In some embodiments, R₂ is substituted C₁-C₁₀ alkyl. In some embodiments, R₂ is unsubstituted C₁-C₁₀ alkyl. In some other embodiments, R₂ is substituted C₁-C₁₀ alkyl. In some embodiments, R₂ is unsubstituted C₂-C₁₀ alkynyl. In some embodiments, R₂ is substituted C₂-C₁₀ alkynyl. In some embodiments, R₂ is unsubstituted C₂-C₁₀ alkenyl. In some embodiments, R₂ is substituted C₂-C₁₀ alkenyl. In some embodiments, R₂ is carboxyl. In some embodiments, R₂ is unsubstituted carbohydrate. In some embodiments, R₂ is substituted carbohydrate. In some embodiments, R₂ is unsubstituted ester. In some embodiments, R₂ is substituted ester. In some embodiments, R₂ is unsubstituted acyloxy. In some embodiments, R₂ is substituted acyloxy. In some embodiments, R₂ is nitro. In some embodiments, R₂ is halogen. In some embodiments, R₂ is unsubstituted C₁-C₁₀ aliphatic acyl. In some embodiments, R₂ is substituted C₁-C₁₀ aliphatic acyl. In some embodiments, R₂ is unsubstituted C₆-C₁₀ aromatic acyl. In some embodiments, R₂ is substituted C₆-C₁₀ aromatic acyl. In some embodiments, R₂ is unsubstituted C₆-C₁₀ aralkyl acyl. In some embodiments, R₂ is substituted C₆-C₁₀ aralkyl acyl. In some embodiments, R₂ is unsubstituted C₆-C₁₀ alkylaryl acyl. In some embodiments, R₂ is substituted C₆-C₁₀ alkylaryl acyl. In some embodiments, R₂ is unsubstituted alkoxy. In some embodiments, R₂ is substituted alkoxy. In some embodiments, R₂ is unsubstituted amine. In some embodiments, R₂ is substituted amine. In some embodiments, R₂ is unsubstituted aryl. In some embodiments, R₂ is substituted aryl. In some embodiments, R₂ is unsubstituted C₄-C₁₀heterocyclyl. In some embodiments, R₂ is substituted C₄-C₁₀heterocyclyl. In some embodiments, R₂ is unsubstituted heteroaryl. In some embodiments, R₂ is substituted heteroaryl. In some embodiments, R₂ is unsubstituted C₃-C₁₀cycloalkyl. In some embodiments, R₂ is substituted C₃-C₁₀cycloalkyl. In some embodiments, R₂ is —OPO₃WY. In some embodiments, R₂ is —OCH₂PO₄WY. In some embodiments, R₂ is —OCH₂PO₄Z. In some embodiments, R₂ is —OPO₃Z.

In some embodiments, R₅ is hydrogen. In some embodiments, R₅ is hydroxyl. In some embodiments, R₅ is carboxaldehyde. In some embodiments, R₅ is unsubstituted amine. In some embodiments, R₅ is substituted amine. In some embodiments, R₅ is unsubstituted C₁-C₁₀ alkyl. In some embodiments, R₅ is substituted C₁-C₁₀ alkyl. In some embodiments, R₅ is unsubstituted C₂-C₁₀ alkynyl. In some embodiments, R₅ is substituted C₂-C₁₀ alkynyl. In some embodiments, R₅ is unsubstituted C₂-C₁₀ alkenyl. In some embodiments, R₅ is substituted C₂-C₁₀ alkenyl. In some embodiments, R₅ is carboxyl. In some embodiments, R₅ is unsubstituted carbohydrate. In some embodiments, R₅ is substituted carbohydrate. In some embodiments, R₅ is unsubstituted ester. In some embodiments, R₅ is substituted ester. In some embodiments, R₅ is unsubstituted acyloxy. In some embodiments, R₅ is substituted acyloxy. In some embodiments, R₅ is nitro. In some embodiments, R₅ is halogen. In some embodiments, R₅ is unsubstituted C₁-C₁₀ aliphatic acyl. In some embodiments, R₅ is substituted C₁-C₁₀ aliphatic acyl. In some embodiments, R₅ is unsubstituted C₆-C₁₀ aromatic acyl. In some embodiments, R₅ is substituted C₆-C₁₀ aromatic acyl. In some embodiments, R₅ is unsubstituted C₆-C₁₀ aralkyl acyl. In some embodiments, R₅ is substituted C₆-C₁₀ aralkyl acyl. In some embodiments, R₅ is unsubstituted C₆-C₁₀ alkylaryl acyl. In some embodiments, R₅ is substituted C₆-C₁₀ alkylaryl acyl. In some embodiments, R₅ is unsubstituted alkoxy. In some embodiments, R₅ is substituted alkoxy. In some embodiments, R₅ is unsubstituted aryl. In some embodiments, R₅ is substituted aryl. In some embodiments, R₅ is unsubstituted C₃-C₁₀heterocyclyl. In some embodiments, R₅ is substituted C₃-C₁₀ heterocyclyl. In some embodiments, R₅ is unsubstituted heteroaryl, In some embodiments, R₅ is unsubstituted C₃-C₁₀cycloalkyl. In some embodiments, R₅ is substituted C₃-C₁₀cycloalkyl. In some embodiments, R₅ is —OPO₃WY. In some embodiments, R₅ is —OCH₂PO₄WY. In some embodiments, R₅ is —OCH₂PO₄Z. In some embodiments, R₅ is —OPO₃Z.

In some embodiments, R₁₂ is hydrogen. In some embodiments, R₁₂ is hydroxyl. In some embodiments, R₁₂ is carboxaldehyde. In some embodiments, R₁₂ is unsubstituted amine. In some embodiments, R₁₂ is substituted amine. In some embodiments, R₁₂ is unsubstituted C₁-C₁₀ alkyl. In some embodiments, R₁₂ is substituted C₁-C₁₀ alkyl. In some embodiments, R₁₂ is unsubstituted C₂-C₁₀ alkynyl. In some embodiments, R₁₂ is substituted C₂-C₁₀ alkynyl. In some embodiments, R₁₂ is unsubstituted C₂-C₁₀ alkenyl. In some embodiments, R₁₂ is substituted C₂-C₁₀ alkenyl. In some embodiments, R₁₂ is carboxyl. In some embodiments, R₁₂ is unsubstituted carbohydrate. In some embodiments, R₁₂ is substituted carbohydrate. In some embodiments, R₁₂ is unsubstituted ester. In some embodiments, R₁₂ is substituted ester. In some embodiments, R₁₂ is unsubstituted acyloxy. In some embodiments, R₁₂ is substituted acyloxy. In some embodiments, R₁₂ is nitro. In some embodiments, R₁₂ is halogen. In some embodiments, R₁₂ is unsubstituted C₁-C₁₀ aliphatic acyl. In some embodiments, R₁₂ is substituted C₁-C₁₀ aliphatic acyl. In some embodiments, R₁₂ is unsubstituted C₆-C₁₀ aromatic acyl. In some embodiments, R₁₂ is substituted C₆-C₁₀ aromatic acyl. In some embodiments, R₁₂ is unsubstituted C₆-C₁₀ aralkyl acyl. In some embodiments, R₁₂ is substituted C₆-C₁₀ aralkyl acyl. In some embodiments, R₁₂ is unsubstituted C₆-C₁₀ alkylaryl acyl. In some embodiments, R₁₂ is substituted C₆-C₁₀ alkylaryl acyl. In some embodiments, R₁₂ is unsubstituted alkoxy. In some embodiments, R₁₂ is substituted alkoxy. In some embodiments, R₁₂ is unsubstituted aryl. In some embodiments, R₁₂ is substituted aryl. In some embodiments, R₁₂ is unsubstituted C₃-C₁₀heterocyclyl. In some embodiments, R₁₂ is substituted C₃-C₁₀heterocyclyl. In some embodiments, R₁₂ is unsubstituted heteroaryl. In some embodiments, R₁₂ is unsubstituted C₃-C₁₀cycloalkyl. In some embodiments, R₁₂ is substituted C₃-C₁₀cycloalkyl. In some embodiments, R₁₂ is —OPO₃WY. In some embodiments, R₁₂ is —OCH₂PO₄WY. In some embodiments, R₁₂ is —OCH₂PO₄Z. In some embodiments, R₁₂ is —OPO₃Z.

In some embodiments, R₁₃ is hydrogen. In some embodiments, R₁₃ is hydroxyl. In some embodiments, R₁₃ is carboxaldehyde. In some embodiments, R₁₃ is unsubstituted amine. In some embodiments, R₁₃ is substituted amine. In some embodiments, R₁₃ is unsubstituted C₁-C₁₀ alkyl. In some embodiments, R₁₃ is substituted C₁-C₁₀ alkyl. In some embodiments, R₁₃ is unsubstituted C₂-C₁₀ alkynyl. In some embodiments, R₁₃ is substituted C₂-C₁₀ alkynyl. In some embodiments, R₁₃ is unsubstituted C₂-C₁₀ alkenyl. In some embodiments, R₁₃ is substituted C₂-C₁₀ alkenyl. In some embodiments, R₁₃ is carboxyl. In some embodiments, R₁₃ is unsubstituted carbohydrate. In some embodiments, R₁₃ is substituted carbohydrate. In some embodiments, R₁₃ is unsubstituted ester. In some embodiments, R₁₃ is substituted ester. In some embodiments, R₁₃ is unsubstituted acyloxy. In some embodiments, R₁₃ is substituted acyloxy. In some embodiments, R₁₃ is nitro. In some embodiments, R₁₃ is halogen. In some embodiments, R₁₃ is unsubstituted C₁-C₁₀ aliphatic acyl. In some embodiments, R₁₃ is substituted C₁-C₁₀ aliphatic acyl. In some embodiments, R₁₃ is unsubstituted C₆-C₁₀ aromatic acyl. In some embodiments, R₁₃ is substituted C₆-C₁₀ aromatic acyl. In some embodiments, R₁₃ is unsubstituted C₆-C₁₀ aralkyl acyl. In some embodiments, R₁₃ is substituted C₆-C₁₀ aralkyl acyl. In some embodiments, R₁₃ is unsubstituted C₆-C₁₀ alkylaryl acyl. In some embodiments, R₁₃ is substituted C₆-C₁₀ alkylaryl acyl. In some embodiments, R₁₃ is unsubstituted alkoxy. In some embodiments, R₁₃ is substituted alkoxy. In some embodiments, R₁₃ is unsubstituted aryl. In some embodiments, R₁₃ is substituted aryl. In some embodiments, R₁₃ is unsubstituted C₃-C₁₀heterocyclyl. In some embodiments, R₁₃ is substituted C₃-C₁₀heterocyclyl. In some embodiments, R₁₃ is unsubstituted heteroaryl, In some embodiments, R₁₃ is unsubstituted C₃-C₁₀cycloalkyl. In some embodiments, R₁₃ is substituted C₃-C₁₀cycloalkyl. In some embodiments, R₁₃ is —OPO₃WY. In some embodiments, R₁₃ is —OCH₂PO₄WY. In some embodiments, R₁₃ is —OCH₂PO₄Z. In some embodiments, R₁₃ is —OPO₃Z.

In some embodiments, R₁₆ is hydrogen. In some embodiments, R₁₆ is unsubstituted C₁-C₁₀ alkyl. In some embodiments, R₁₆ is substituted C₁-C₁₀ alkyl. In some embodiments, R₁₆ is unsubstituted C₂-C₁₀ alkynyl. In some embodiments, R₁₆ is substituted C₂-C₁₀ alkynyl. In some embodiments, R₁₆ is unsubstituted C₂-C₁₀ alkenyl. In some embodiments, R₁₆ is substituted C₂-C₁₀ alkenyl. In some embodiments, R₁₆ is unsubstituted carbohydrate. In some embodiments, R₁₆ is substituted carbohydrate. In some embodiments, R₁₆ is unsubstituted C₁-C₁₀ aliphatic acyl. In some embodiments, R₁₆ is substituted C₁-C₁₀ aliphatic acyl. In some embodiments, R₁₆ is unsubstituted C₆-C₁₀ aromatic acyl. In some embodiments, R₁₆ is substituted C₆-C₁₀ aromatic acyl. In some embodiments, R₁₆ is unsubstituted C₆-C₁₀ aralkyl acyl. In some embodiments, R₁₆ is substituted C₆-C₁₀ aralkyl acyl. In some embodiments, R₁₆ is unsubstituted C₆-C₁₀ alkylaryl acyl. In some embodiments, R₁₆ is substituted C₆-C₁₀ alkylaryl acyl. In some embodiments, R₁₆ is unsubstituted aryl. In some embodiments, R₁₆ is substituted aryl. In some embodiments, R₁₆ is unsubstituted C₃-C₁₀heterocyclyl. In some embodiments, R₁₆ is substituted C₃-C₁₀ heterocyclyl. In some embodiments, R₁₆ is unsubstituted heteroaryl. In some embodiments, R₁₆ is substituted heteroaryl. In some embodiments, R₁₆ is unsubstituted C₃-C₁₀cycloalkyl. In some embodiments, R₁₆ is substituted C₃-C₁₀cycloalkyl. In some embodiments, R₁₆ is —PO₃WY. In some embodiments, R₁₆ is —CH₂PO₄WY. In some embodiments, R₁₆ is —CH₂PO₄Z. In some embodiments, R₁₆ is —PO₃Z.

In some embodiments, R₁₇ is hydrogen. In some embodiments, R₁₇ is hydroxy. In some embodiments, R₁₇ is carboxaldehyde. In some embodiments, R₁₇ is unsubstituted amine. In some embodiments, R₁₇ is substituted amine. In some embodiments, R₁₇ is unsubstituted C₁-C₁₀ alkyl. In some embodiments, R₁₇ is unsubstituted C₂-C₁₀ alkynyl. In some embodiments, R₁₇ is substituted C₂-C₁₀ alkynyl. In some embodiments, R₁₇ is unsubstituted C₂-C₁₀ alkenyl. In some embodiments, R₁₇ is substituted C₂-C₁₀ alkenyl. In some embodiments, R₁₇ is carboxyl. In some embodiments, R₁₇ is unsubstituted carbohydrate. In some embodiments, R₁₇ is substituted carbohydrate. In some embodiments, R₁₇ is unsubstituted ester. In some embodiments, R₁₇ is substituted ester. In some embodiments, R₁₇ is unsubstituted acyloxy. In some embodiments, R₁₇ is substituted acyloxy. In some embodiments, R₁₇ is nitro. In some embodiments, R₁₇ is halogen. In some embodiments, R₁₇ is unsubstituted C₁-C₁₀ aliphatic acyl. In some embodiments, R₁₇ is substituted C₁-C₁₀ aliphatic acyl. In some embodiments, R₁₇ is unsubstituted C₆-C₁₀ aromatic acyl. In some embodiments, R₁₇ is substituted C₆-C₁₀ aromatic acyl. In some embodiments, R₁₇ is unsubstituted C₆-C₁₀ aralkyl acyl. In some embodiments, R₁₇ is substituted C₆-C₁₀ aralkyl acyl. In some embodiments, R₁₇ is unsubstituted C₆-C₁₀ alkylaryl acyl. In some embodiments, R₁₇ is substituted C₆-C₁₀ alkylaryl acyl. In some embodiments, R₁₇ is unsubstituted alkoxy. In some embodiments, R₁₇ is substituted alkoxy. In some embodiments, R₁₇ is unsubstituted aryl. In some embodiments, R₁₇ is substituted aryl. In some embodiments, R₁₇ is unsubstituted C₃-C₁₀heterocyclyl. In some embodiments, R₁₇ is substituted C₃-C₁₀heterocyclyl. In some embodiments, R₁₇ is unsubstituted heteroaryl. In some embodiments, R₁₇ is substituted heteroaryl. In some embodiments, R₁₇ is unsubstituted C₃-C₁₀cycloalkyl. In some embodiments, R₁₇ is substituted C₃-C₁₀cycloalkyl. In some embodiments, R₁₇ is —OPO₃WY. In some embodiments, R₁₇ is —OCH₂PO₄WY. In some embodiments, R₁₇ is —OCH₂PO₄Z. In some embodiments, R₁₇ is —OPO₃Z.

In some embodiments, R₁₈ is hydrogen. In some embodiments, R₁₈ is hydroxy. In some embodiments, R₁₈ is carboxaldehyde. In some embodiments, R₁₈ is unsubstituted amine. In some embodiments, R₁₈ is substituted amine. In some embodiments, R₁₈ is unsubstituted C₁-C₁₀ alkyl. In some embodiments, R₁₈ is unsubstituted C₂-C₁₀ alkynyl. In some embodiments, R₁₈ is substituted C₂-C₁₀ alkynyl. In some embodiments, R₁₈ is unsubstituted C₂-C₁₀ alkenyl. In some embodiments, R₁₈ is substituted C₂-C₁₀ alkenyl. In some embodiments, R₁₈ is carboxyl. In some embodiments, R₁₈ is unsubstituted carbohydrate. In some embodiments, R₁₈ is substituted carbohydrate. In some embodiments, R₁₈ is substituted carbohydrate. In some embodiments, R₁₈ is unsubstituted ester. In some embodiments, R₁₈ is substituted ester. In some embodiments, R₁₈ is unsubstituted acyloxy. In some embodiments, R₁₈ is substituted acyloxy. In some embodiments, R₁₈ is nitro. In some embodiments, R₁₈ is halogen. In some embodiments, R₁₈ is unsubstituted C₁-C₁₀ aliphatic acyl. In some embodiments, R₁₈ is substituted C₁-C₁₀ aliphatic acyl. In some embodiments, R₁₈ is unsubstituted C₆-C₁₀ aromatic acyl. In some embodiments, R₁₈ is substituted C₆-C₁₀ aromatic acyl. In some embodiments, R₁₈ is unsubstituted C₆-C₁₀ aralkyl acyl. In some embodiments, R₁₈ is substituted C₆-C₁₀ aralkyl acyl. In some embodiments, R₁₈ is unsubstituted C₆-C₁₀ alkylaryl acyl. In some embodiments, R₁₈ is substituted C₆-C₁₀ alkylaryl acyl. In some embodiments, R₁₈ is unsubstituted alkoxy. In some embodiments, R₁₈ is substituted alkoxy. In some embodiments, R₁₈ is unsubstituted aryl. In some embodiments, R₁₈ is substituted aryl. In some embodiments, R₁₈ is unsubstituted C₃-C₁₀heterocyclyl. In some embodiments, R₁₈ is substituted C₃-C₁₀heterocyclyl. In some embodiments, R₁₈ is unsubstituted heteroaryl. In some embodiments, R₁₈ is substituted heteroaryl. In some embodiments, R₁₈ is unsubstituted C₃-C₁₀cycloalkyl. In some embodiments, R₁₈ is substituted C₃-C₁₀cycloalkyl. In some embodiments, R₁₈ is —OPO₃WY. In some embodiments, R₁₈ is —OCH₂PO₄WY. In some embodiments, R₁₈ is —OCH₂PO₄Z. In some embodiments, R₁₈ is —OPO₃Z.

In some embodiments, R₁₉ is hydrogen. In some embodiments, R₁₉ is unsubstituted C₁-C₁₀ alkyl. In some embodiments, R₁₉ is substituted C₁-C₁₀ alkyl. In some embodiments, R₁₉ is unsubstituted C₂-C₁₀ alkynyl. In some embodiments, R₁₉ is substituted C₂-C₁₀ alkynyl. In some embodiments, R₁₉ is unsubstituted C₂-C₁₀ alkenyl. In some embodiments, R₁₉ is substituted C₂-C₁₀ alkenyl. In some embodiments, R₁₉ is unsubstituted carbohydrate. In some embodiments, R₁₉ is substituted carbohydrate. In some embodiments, R₁₉ is unsubstituted C₁-C₁₀ aliphatic acyl. In some embodiments, R₁₉ is substituted C₁-C₁₀ aliphatic acyl. In some embodiments, R₁₉ is unsubstituted C₆-C₁₀ aromatic acyl. In some embodiments, R₁₉ is substituted C₆-C₁₀ aromatic acyl. In some embodiments, R₁₉ is unsubstituted C₆-C₁₀ aralkyl acyl. In some embodiments, R₁₉ is substituted C₆-C₁₀ aralkyl acyl. In some embodiments, R₁₉ is unsubstituted C₆-C₁₀ alkylaryl acyl. In some embodiments, R₁₉ is substituted C₆-C₁₀ alkylaryl acyl. In some embodiments, R₁₉ is unsubstituted aryl. In some embodiments, R₁₉ is substituted aryl. In some embodiments, R₁₉ is unsubstituted C₃-C₁₀heterocyclyl. In some embodiments, R₁₉ is substituted C₃-C₁₀ heterocyclyl. In some embodiments, R₁₉ is unsubstituted heteroaryl. In some embodiments, R₁₉ is substituted heteroaryl. In some embodiments, R₁₉ is unsubstituted C₃-C₁₀cycloalkyl. In some embodiments, R₁₉ is substituted C₃-C₁₀cycloalkyl. In some embodiments, R₁₉ is —PO₃WY. In some embodiments, R₁₉ is —CH₂PO₄WY. In some embodiments, R₁₉ is —CH₂PO₄Z. In some embodiments, R₁₉ is —PO₃Z.

In some embodiments, R₂₁ is hydrogen. In some embodiments, R₂₁ is hydroxy. In some embodiments, R₂₁ is carboxaldehyde. In some embodiments, R₂₁ is unsubstituted amine. In some embodiments, R₂₁ is substituted amine. In some embodiments, R₂₁ is unsubstituted C₁-C₁₀ alkyl. In some embodiments, R₂₁ is unsubstituted C₂-C₁₀ alkynyl. In some embodiments, R₂₁ is substituted C₂-C₁₀ alkynyl. In some embodiments, R₂₁ is unsubstituted C₂-C₁₀ alkenyl. In some embodiments, R₂₁ is substituted C₂-C₁₀ alkenyl. In some embodiments, R₂₁ is carboxyl. In some embodiments, R₂₁ is unsubstituted carbohydrate. In some embodiments, R₂₁ is substituted carbohydrate. In some embodiments, R₂₁ is unsubstituted ester. In some embodiments, R₂₁ is substituted ester. In some embodiments, R₂₁ is unsubstituted acyloxy. In some embodiments, R₂₁ is substituted acyloxy. In some embodiments, R₂₁ is nitro. In some embodiments, R₂₁ is halogen. In some embodiments, R₂₁ is unsubstituted C₁-C₁₀ aliphatic acyl. In some embodiments, R₂₁ is substituted C₁-C₁₀ aliphatic acyl. In some embodiments, R₂₁ is unsubstituted C₆-C₁₀ aromatic acyl. In some embodiments, R₂₁ is substituted C₆-C₁₀ aromatic acyl. In some embodiments, R₂₁ is unsubstituted C₆-C₁₀ aralkyl acyl. In some embodiments, R₂₁ is substituted C₆-C₁₀ aralkyl acyl. In some embodiments, R₂₁ is unsubstituted C₆-C₁₀ alkylaryl acyl. In some embodiments, R₂₁ is substituted C₆-C₁₀ alkylaryl acyl. In some embodiments, R₂₁ is unsubstituted alkoxy. In some embodiments, R₂₁ is substituted alkoxy. In some embodiments, R₂₁ is unsubstituted aryl. In some embodiments, R₂₁ is substituted aryl. In some embodiments, R₂₁ is unsubstituted C₃-C₁₀heterocyclyl. In some embodiments, R₂₁ is substituted C₃-C₁₀heterocyclyl. In some embodiments, R₂₁ is unsubstituted heteroaryl. In some embodiments, R₂₁ is substituted heteroaryl. In some embodiments, R₂₁ is unsubstituted C₃-C₁₀cycloalkyl. In some embodiments, R₂₁ is substituted C₃-C₁₀cycloalkyl. In some embodiments, R₂₁ is —OPO₃WY. In some embodiments, R₂₁ is —OCH₂PO₄WY. In some embodiments, R₂₁ is —OCH₂PO₄Z. In some embodiments, R₂₁ is —OPO₃Z.

In some embodiments, s is an integer of 0. In some embodiments, s is an integer of 1. In some embodiments, s is an integer of 2. In some embodiments, s is an integer of 3.

In some embodiments, n is an integer of 0. In some embodiments, n is an integer of 1. In some embodiments, n is an integer of 2. In some embodiments, n is an integer of 3. In some embodiments, n is an integer of 4.

In various embodiments, W is hydrogen. In various embodiments, W is unsubstituted methyl. In various embodiments, W is substituted methyl. In various embodiments, W is unsubstituted ethyl. In various embodiments, W is substituted ethyl. In various embodiments, W is unsubstituted alkyl. In various embodiments, W is substituted alkyl. In various embodiments, W is unsubstituted carbohydrate. In various embodiments, W is substituted carbohydrate. In various embodiments, W is potassium. In various embodiments, W is sodium. In various embodiments, W is lithium.

In various embodiments, Y is hydrogen. In various embodiments, Y is unsubstituted methyl. In various embodiments, Y is substituted methyl. In various embodiments, Y is unsubstituted ethyl. In various embodiments, Y is substituted ethyl. In various embodiments, Y is unsubstituted alkyl. In various embodiments, Y is substituted alkyl. In various embodiments, Y is unsubstituted carbohydrate. In various embodiments, Y is substituted carbohydrate. In various embodiments, Y is potassium. In various embodiments, Y is sodium. In various embodiments, Y is lithium.

In various embodiments, Z is calcium. In various embodiments, Z is magnesium. In various embodiments, Z is iron.

In some embodiments of the invention, the pyrone analog of Formula V is of Formula XXIX or Formula XXX or a pharmaceutically/veterinarily acceptable salt thereof:

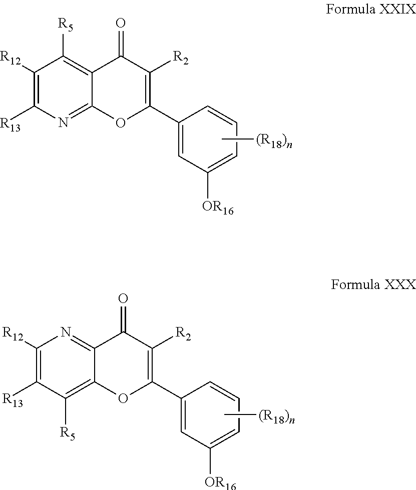

wherein R₁₆ is hydrogen, —PO₃WY, —CH₂PO₄WY, —CH₂PO₄Z or —PO₃Z;

wherein each instance of R₁₈ is independently hydrogen, hydroxyl, carboxaldehyde, amine, C₁-C₁₀ alkyl, C₂-C₁₀ alkynyl, C₂-C₁₀ alkenyl, carboxyl, carbohydrate, ester, acyloxy, nitro, halogen, C₁-C₁₀ aliphatic acyl, C₆-C₁₀ aromatic acyl, C₆-C₁₀ aralkyl acyl, C₆-C₁₀ alkylaryl acyl, alkoxy, alkyl, phosphate, aryl, heteroaryl, C₃-C₁₀ heterocyclic, C₃-C₁₀cycloalkyl, —OPO₃WY, —OCH₂PO₄WY, —OCH₂PO₄Z or —OPO₃Z; and

n is an integer of 0, 1, 2, 3, or 4.

In some embodiments of the invention, R₁₆ is —PO₃WY, —CH₂PO₄WY, —CH₂PO₄Z or —PO₃Z, or at least one of R₁₈ is —OPO₃WY, —OCH₂PO₄WY, —OCH₂PO₄Z or —OPO₃Z. In some embodiments, R₁₆ is —PO₃WY, —CH₂PO₄WY, —CH₂PO₄Z or —PO₃Z and at least one of R₁₈ is —OPO₃WY, —OCH₂PO₄WY, —OCH₂PO₄Z or —OPO₃Z.

In some embodiments of the invention, the pyrone analog of Formula V is of Formula XXXI or a pharmaceutically/veterinarily acceptable salt thereof:

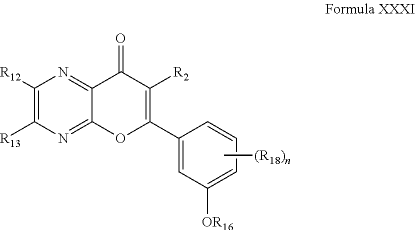

wherein R₁₆ is hydrogen, —PO₃WY, —CH₂PO₄WY, —CH₂PO₄Z or —PO₃Z;

wherein each instance of R₁₈ is independently hydrogen, hydroxyl, carboxaldehyde, amine, C₁-C₁₀ alkyl, C₂-C₁₀ alkynyl, C₂-C₁₀ alkenyl, carboxyl, carbohydrate, ester, acyloxy, nitro, halogen, C₁-C₁₀ aliphatic acyl, C₆-C₁₀ aromatic acyl, C₆-C₁₀ aralkyl acyl, C₆-C₁₀ alkylaryl acyl, alkoxy, alkyl, phosphate, aryl, heteroaryl, C₃-C₁₀ heterocyclic, C₃-C₁₀cycloalkyl, —OPO₃WY, —OCH₂PO₄WY, —OCH₂PO₄Z or —OPO₃Z; and

n is an integer of 0, 1, 2, 3, or 4.

In some embodiments of the invention, R₁₆ is —PO₃WY, —CH₂PO₄WY, —CH₂PO₄Z or —PO₃Z, or at least one of R₁₈ is —OPO₃WY, —OCH₂PO₄WY, —OCH₂PO₄Z or —OPO₃Z. In some embodiments, R₁₆ is —PO₃WY, —CH₂PO₄WY, —CH₂PO₄Z or —PO₃Z and at least one of R₁₈ is —OPO₃WY, —OCH₂PO₄WY, —OCH₂PO₄Z or —OPO₃Z.

In some embodiments of the invention, the pyrone analog of Formula II is of Formula VI or a pharmaceutically/veterinarily acceptable salt thereof:

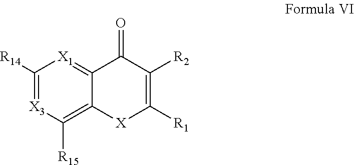

wherein X is O, S, or NR′, wherein R′ is hydrogen, C₁-C₁₀ alkyl, C₂-C₁₀ alkynyl, C₂-C₁₀ alkenyl, C₁-C₁₀ aliphatic acyl, C₆-C₁₀ aromatic acyl, C₆-C₁₀ aralkyl acyl, C₆-C₁₀ alkylaryl acyl, aryl, C₃-C₁₀ heterocyclyl, heteroaryl, or C₃-C₁₀cycloalkyl;

R₁, and R₂ are independently hydrogen, hydroxyl, C₁-C₁₀ alkyl, C₂-C₁₀ alkynyl, C₂-C₁₀ alkenyl, carboxyl, carbohydrate, ester, acyloxy, nitro, halogen, C₁-C₁₀ aliphatic acyl, C₆-C₁₀ aromatic acyl, C₆-C₁₀ aralkyl acyl, C₆-C₁₀ alkylaryl acyl, alkoxy, amine, aryl, C₄-C₁₀heterocyclyl, heteroaryl, C₃-C₁₀cycloalkyl, —OPO₃WY, —OCH₂PO₄WY, —OCH₂PO₄Z or —OPO₃Z;

X₁ and X₄ are independently CR₅, O, S, or N;

each instance of R₅ is independently hydrogen, hydroxyl, carboxaldehyde, amino, C₁-C₁₀ alkyl, C₂-C₁₀ alkynyl, C₂-C₁₀ alkenyl, carboxyl, carbohydrate, ester, acyloxy, nitro, halogen, C₁-C₁₀ aliphatic acyl, C₆-C₁₀ aromatic acyl, C₆-C₁₀ aralkyl acyl, C₆-C₁₀alkylaryl acyl, alkoxy, amine, aryl, C₃-C₁₀heterocyclyl, heteroaryl, C₃-C₁₀cycloalkyl, —OPO₃WY, —OCH₂PO₄WY, —OCH₂PO₄Z or —OPO₃Z;

R₁₄ and R₁₅ are independently hydrogen, hydroxyl, carboxaldehyde, amino, C₁-C₁₀ alkyl, C₂-C₁₀ alkynyl, C₂-C₁₀ alkenyl, carboxyl, carbohydrate, ester, acyloxy, nitro, halogen, C₁-C₁₀ aliphatic acyl, C₆-C₁₀ aromatic acyl, C₆-C₁₀ aralkyl acyl, C₆-C₁₀ alkylaryl acyl, alkoxy, amine, aryl, C₃-C₁₀ heterocyclyl, heteroaryl, C₃-C₁₀cycloalkyl, —OPO₃WY, —OCH₂PO₄WY, —OCH₂PO₄Z or —OPO₃Z;

W and Y are independently hydrogen, methyl, ethyl, alkyl, carbohydrate, or a cation, and Z is a multivalent cation; and

wherein the compound of Formula VI is not the compound of Formula A:

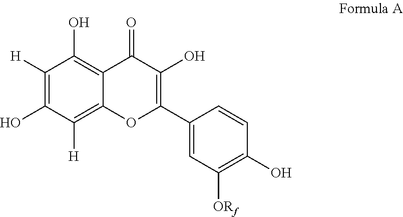

wherein R_{f} is hydrogen.

In some embodiments, the compound of Formula VI is not the compound of Formula A wherein R_{f} is PO₃K₂.

In some embodiments, X is O.

In other embodiments, X is S.

In yet other embodiments, X is NR′.

In some embodiments, R′ is hydrogen. In some embodiments, R′ is unsubstituted C₁-C₁₀ alkyl. In some embodiments, R′ is substituted C₁-C₁₀ alkyl. In some embodiments, R′ is unsubstituted C₂-C₁₀ alkynyl. In some embodiments, R′ is substituted C₂-C₁₀ alkynyl. In some embodiments, R′ is unsubstituted C₂-C₁₀ alkenyl. In some embodiments, R′ is substituted C₂-C₁₀ alkenyl. In some embodiments, R′ is unsubstituted C₂-C₁₀ alkenyl. In some embodiments, R′ is substituted C₂-C₁₀ alkenyl. In some embodiments, R′ is unsubstituted C₁-C₁₀ aliphatic acyl. In some embodiments, R′ is substituted C₁-C₁₀ aliphatic acyl. In some embodiments, R′ is unsubstituted C₆-C₁₀ aromatic acyl. In some embodiments, R′ is substituted C₆-C₁₀ aromatic acyl. In some embodiments, R′ is unsubstituted C₆-C₁₀ aralkyl acyl. In some embodiments, R′ is substituted C₆-C₁₀ aralkyl acyl. In some embodiments, R′ is unsubstituted C₆-C₁₀ alkylaryl acyl. In some embodiments, R′ is substituted C₆-C₁₀ alkylaryl acyl. In some embodiments, R′ is unsubstituted aryl. In some embodiments, R′ is substituted aryl. In some embodiments, R′ is unsubstituted C₃-C₁₀ heterocyclyl. In some embodiments, R′ is substituted C₃-C₁₀ heterocyclyl. In some embodiments, R′ is unsubstituted heteroaryl. In some embodiments, R′ is substituted heteroaryl. In some embodiments, R′ is unsubstituted C₃-C₁₀cycloalkyl. In some embodiments, R′ is substituted C₃-C₁₀cycloalkyl.

In some embodiments, X₁ is CR₅.

In other embodiments, X₁ is O.

In yet other embodiments, X₁ is S.

In further embodiments, X₁ is N.

In other embodiments, X₃ is CR₅.

In some embodiments, X₃ is O.

In yet other embodiments, X₃ is S.

In some embodiments, X₃ is N.

In some embodiments, at least one of X₁ or X₃ is N.

In some embodiments, R₁ is hydrogen. In some embodiments, R₁ is hydroxyl. In some embodiments, R₁ is optionally substituted C₁-C₁₀ alkyl. In some embodiments, R₁ is unsubstituted C₁-C₁₀ alkyl. In some embodiments, R₁ is substituted C₁-C₁₀ alkyl. In some embodiments, R₁ is unsubstituted C₁-C₁₀ alkyl. In some other embodiments, R₁ is substituted C₁-C₁₀ alkyl. In some embodiments, R₁ is unsubstituted C₂-C₁₀ alkynyl. In some embodiments, R₁ is substituted C₂-C₁₀ alkynyl. In some embodiments, R₁ is unsubstituted C₂-C₁₀ alkenyl. In some embodiments, R₁ is substituted C₂-C₁₀ alkenyl. In some embodiments, R₁ is carboxyl. In some embodiments, R₁ is unsubstituted carbohydrate. In some embodiments, R₁ is substituted carbohydrate. In some embodiments, R₁ is unsubstituted ester. In some embodiments, R₁ is substituted ester. In some embodiments, R₁ is unsubstituted acyloxy. In some embodiments, R₁ is substituted acyloxy. In some embodiments, R₁ is nitro. In some embodiments, R₁ is halogen. In some embodiments, R₁ is unsubstituted C₁-C₁₀ aliphatic acyl. In some embodiments, R₁ is substituted C₁-C₁₀ aliphatic acyl. In some embodiments, R₁ is unsubstituted C₆-C₁₀ aromatic acyl. In some embodiments, R₁ is substituted C₆-C₁₀ aromatic acyl. In some embodiments, R₁ is unsubstituted C₆-C₁₀ aralkyl acyl. In some embodiments, R₁ is substituted C₆-C₁₀ aralkyl acyl. In some embodiments, R₁ is unsubstituted C₆-C₁₀ alkylaryl acyl. In some embodiments, R₁ is substituted C₆-C₁₀ alkylaryl acyl. In some embodiments, R₁ is unsubstituted alkoxy. In some embodiments, R₁ is substituted alkoxy. In some embodiments, R₁ is unsubstituted amine. In some embodiments, R₁ is substituted amine. In some embodiments, R₁ is unsubstituted aryl. In some embodiments, R₁ is substituted aryl. In some embodiments, R₁ is unsubstituted C₄-C₁₀heterocyclyl. In some embodiments, R₁ is substituted C₄-C₁₀heterocyclyl. In some embodiments, R₁ is unsubstituted heteroaryl. In some embodiments, R₁ is substituted heteroaryl. In some embodiments, R₁ is unsubstituted C₃-C₁₀cycloalkyl. In some embodiments, R₁ is substituted C₃-C₁₀cycloalkyl. In some embodiments, R₁ is —OPO₃WY. In some embodiments, R₁ is —OCH₂PO₄WY. In some embodiments, R₁ is —OCH₂PO₄Z. In some embodiments, R₁ is —OPO₃Z.

In some embodiments, when R₁ is aryl, it is monocyclic. In some embodiments, when R₁ is aryl, it is bicyclic. In some embodiments, when R₁ is heteroaryl, it is monocyclic. In some embodiments, when R₁ is heteroaryl, it is bicyclic.

In various embodiments, R₁ is one of the following formulae:

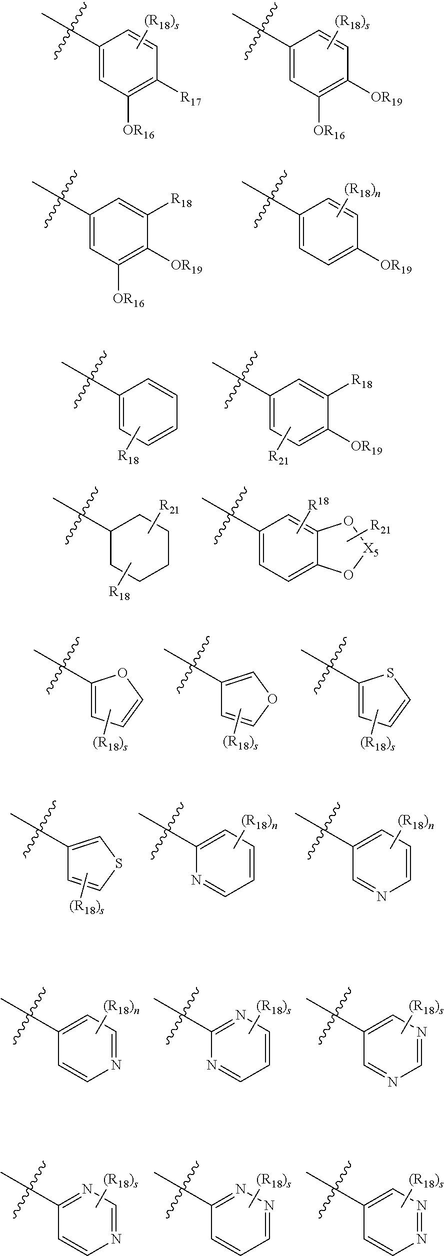

wherein R₁₆ is hydrogen, C₁-C₁₀ alkyl, C₂-C₁₀ alkynyl, C₂-C₁₀ alkenyl, carbohydrate, C₁-C₁₀ aliphatic acyl, C₆-C₁₀ aromatic acyl, C₆-C₁₀ aralkyl acyl, C₆-C₁₀ alkylaryl acyl, aryl, C₃-C₁₀ heterocyclyl, heteroaryl, C₃-C₁₀cycloalkyl, —PO₃WY, —CH₂PO₄WY, —CH₂PO₄Z or —PO₃Z;

R₁₇ is hydrogen, hydroxy, carboxaldehyde, amine, C₁-C₁₀ alkyl, C₂-C₁₀ alkynyl, C₂-C₁₀ alkenyl, carboxyl, carbohydrate, ester, acyloxy, nitro, halogen, C₁-C₁₀ aliphatic acyl, C₆-C₁₀ aromatic acyl, C₆-C₁₀ aralkyl acyl, C₆-C₁₀ alkylaryl acyl, alkoxy, aryl, C₃-C₁₀ heterocyclyl, heteroaryl, or C₃-C₁₀cycloalkyl, —OPO₃WY, —OCH₂PO₄WY, —OCH₂PO₄Z or —OPO₃Z;

each instance of R₁₈ and R₂₁ is independently hydrogen, hydroxyl, carboxaldehyde, amine, C₁-C₁₀ alkyl, C₂-C₁₀ alkynyl, C₂-C₁₀ alkenyl, carboxyl, carbohydrate, ester, acyloxy, nitro, halogen, C₁-C₁₀ aliphatic acyl, C₆-C₁₀ aromatic acyl, C₆-C₁₀ aralkyl acyl, C₆-C₁₀ alkylaryl acyl, alkoxy, alkyl, phosphate, aryl, heteroaryl, C₃-C₁₀ heterocyclic, C₃-C₁₀cycloalkyl, —OPO₃WY, —OCH₂PO₄WY, —OCH₂PO₄Z or —OPO₃Z;

R₁₉ is hydrogen, C₁-C₁₀ alkyl, C₂-C₁₀ alkynyl, C₂-C₁₀ alkenyl, carbohydrate, C₁-C₁₀ aliphatic acyl, C₆-C₁₀ aromatic acyl, C₆-C₁₀ aralkyl acyl, C₆-C₁₀ alkylaryl acyl, aryl, C₃-C₁₀heterocyclyl, heteroaryl, optionally substituted C₃-C₁₀cycloalkyl, —PO₃WY, —CH₂PO₄WY, —CH₂PO₄Z or —PO₃Z;

s is an integer of 0, 1, 2, or 3; and

n is an integer of 0, 1, 2, 3, or 4.

In some embodiments of the invention, at least one of R₁₆ and R₁₉ is —PO₃WY, —CH₂PO₄WY, —CH₂PO₄Z or —PO₃Z, or at least one of R₁₇ or R₁₈ is —OPO₃WY, —OCH₂PO₄WY, —OCH₂PO₄Z or —OPO₃Z. In some embodiments, at least one of R₁₆ and R₁₉ is —PO₃WY, —CH₂PO₄WY, —CH₂PO₄Z or —PO₃Z and at least one of R₁₇ or R₁₈ is —OPO₃WY, —OCH₂PO₄WY, —OCH₂PO₄Z or —OPO₃Z.

In some embodiments, R₂ is hydrogen. In some embodiments, R₂ is hydroxyl. In some embodiments, R₂ is optionally substituted C₁-C₁₀ alkyl. In some embodiments, R₂ is unsubstituted C₁-C₁₀ alkyl. In some embodiments, R₂ is substituted C₁-C₁₀ alkyl. In some embodiments, R₂ is unsubstituted C₁-C₁₀ alkyl. In some other embodiments, R₂ is substituted C₁-C₁₀ alkyl. In some embodiments, R₂ is unsubstituted C₂-C₁₀ alkynyl. In some embodiments, R₂ is substituted C₂-C₁₀ alkynyl. In some embodiments, R₂ is unsubstituted C₂-C₁₀ alkenyl. In some embodiments, R₂ is substituted C₂-C₁₀ alkenyl. In some embodiments, R₂ is carboxyl. In some embodiments, R₂ is unsubstituted carbohydrate. In some embodiments, R₂ is substituted carbohydrate. In some embodiments, R₂ is unsubstituted ester. In some embodiments, R₂ is substituted ester. In some embodiments, R₂ is unsubstituted acyloxy. In some embodiments, R₂ is substituted acyloxy. In some embodiments, R₂ is nitro. In some embodiments, R₂ is halogen. In some embodiments, R₂ is unsubstituted C₁-C₁₀ aliphatic acyl. In some embodiments, R₂ is substituted C₁-C₁₀ aliphatic acyl. In some embodiments, R₂ is unsubstituted C₆-C₁₀ aromatic acyl. In some embodiments, R₂ is substituted C₆-C₁₀ aromatic acyl. In some embodiments, R₂ is unsubstituted C₆-C₁₀ aralkyl acyl. In some embodiments, R₂ is substituted C₆-C₁₀ aralkyl acyl. In some embodiments, R₂ is unsubstituted C₆-C₁₀ alkylaryl acyl. In some embodiments, R₂ is substituted C₆-C₁₀ alkylaryl acyl. In some embodiments, R₂ is unsubstituted alkoxy. In some embodiments, R₂ is substituted alkoxy. In some embodiments, R₂ is unsubstituted amine. In some embodiments, R₂ is substituted amine. In some embodiments, R₂ is unsubstituted aryl. In some embodiments, R₂ is substituted aryl. In some embodiments, R₂ is unsubstituted C₄-C₁₀heterocyclyl. In some embodiments, R₂ is substituted C₄-C₁₀heterocyclyl. In some embodiments, R₂ is unsubstituted heteroaryl. In some embodiments, R₂ is substituted heteroaryl. In some embodiments, R₂ is unsubstituted C₃-C₁₀cycloalkyl. In some embodiments, R₂ is substituted C₃-C₁₀cycloalkyl. In some embodiments, R₂ is —OPO₃WY. In some embodiments, R₂ is —OCH₂PO₄WY. In some embodiments, R₂ is —OCH₂PO₄Z. In some embodiments, R₂ is —OPO₃Z.

In some embodiments, R₅ is hydrogen. In some embodiments, R₅ is hydroxyl. In some embodiments, R₅ is carboxaldehyde. In some embodiments, R₅ is unsubstituted amine. In some embodiments, R₅ is substituted amine. In some embodiments, R₅ is unsubstituted C₁-C₁₀ alkyl. In some embodiments, R₅ is substituted C₁-C₁₀ alkyl. In some embodiments, R₅ is unsubstituted C₂-C₁₀ alkynyl. In some embodiments, R₅ is substituted C₂-C₁₀ alkynyl. In some embodiments, R₅ is unsubstituted C₂-C₁₀ alkenyl. In some embodiments, R₅ is substituted C₂-C₁₀ alkenyl. In some embodiments, R₅ is carboxyl. In some embodiments, R₅ is unsubstituted carbohydrate. In some embodiments, R₅ is substituted carbohydrate. In some embodiments, R₅ is unsubstituted ester. In some embodiments, R₅ is substituted ester. In some embodiments, R₅ is unsubstituted acyloxy. In some embodiments, R₅ is substituted acyloxy. In some embodiments, R₅ is nitro. In some embodiments, R₅ is halogen. In some embodiments, R₅ is unsubstituted C₁-C₁₀ aliphatic acyl. In some embodiments, R₅ is substituted C₁-C₁₀ aliphatic acyl. In some embodiments, R₅ is unsubstituted C₆-C₁₀ aromatic acyl. In some embodiments, R₅ is substituted C₆-C₁₀ aromatic acyl. In some embodiments, R₅ is unsubstituted C₆-C₁₀ aralkyl acyl. In some embodiments, R₅ is substituted C₆-C₁₀ aralkyl acyl. In some embodiments, R₅ is unsubstituted C₆-C₁₀ alkylaryl acyl. In some embodiments, R₅ is substituted C₆-C₁₀ alkylaryl acyl. In some embodiments, R₅ is unsubstituted alkoxy. In some embodiments, R₅ is substituted alkoxy. In some embodiments, R₅ is unsubstituted aryl. In some embodiments, R₅ is substituted aryl. In some embodiments, R₅ is unsubstituted C₃-C₁₀heterocyclyl. In some embodiments, R₅ is substituted C₃-C₁₀ heterocyclyl. In some embodiments, R₅ is unsubstituted heteroaryl, In some embodiments, R₅ is unsubstituted C₃-C₁₀cycloalkyl. In some embodiments, R₅ is substituted C₃-C₁₀cycloalkyl. In some embodiments, R₅ is —OPO₃WY. In some embodiments, R₅ is —OCH₂PO₄WY. In some embodiments, R₅ is —OCH₂PO₄Z. In some embodiments, R₅ is —OPO₃Z.

In some embodiments, R₁₄ is hydrogen. In some embodiments, R₁₄ is hydroxyl. In some embodiments, R₁₄ is carboxaldehyde. In some embodiments, R₁₄ is unsubstituted amine. In some embodiments, R₁₄ is substituted amine. In some embodiments, R₁₄ is unsubstituted C₁-C₁₀ alkyl. In some embodiments, R₁₄ is substituted C₁-C₁₀ alkyl. In some embodiments, R₁₄ is unsubstituted C₂-C₁₀ alkynyl. In some embodiments, R₁₄ is substituted C₂-C₁₀ alkynyl. In some embodiments, R₁₄ is unsubstituted C₂-C₁₀ alkenyl. In some embodiments, R₁₄ is substituted C₂-C₁₀ alkenyl. In some embodiments, R₁₄ is carboxyl. In some embodiments, R₁₄ is unsubstituted carbohydrate. In some embodiments, R₁₄ is substituted carbohydrate. In some embodiments, R₁₄ is unsubstituted ester. In some embodiments, R₁₄ is substituted ester. In some embodiments, R₁₄ is unsubstituted acyloxy. In some embodiments, R₁₄ is substituted acyloxy. In some embodiments, R₁₄ is nitro. In some embodiments, R₁₄ is halogen. In some embodiments, R₁₄ is unsubstituted C₁-C₁₀ aliphatic acyl. In some embodiments, R₁₄ is substituted C₁-C₁₀ aliphatic acyl. In some embodiments, R₁₄ is unsubstituted C₆-C₁₀ aromatic acyl. In some embodiments, R₁₄ is substituted C₆-C₁₀ aromatic acyl. In some embodiments, R₁₄ is unsubstituted C₆-C₁₀ aralkyl acyl. In some embodiments, R₁₄ is substituted C₆-C₁₀ aralkyl acyl. In some embodiments, R₁₄ is unsubstituted C₆-C₁₀ alkylaryl acyl. In some embodiments, R₁₄ is substituted C₆-C₁₀ alkylaryl acyl. In some embodiments, R₁₄ is unsubstituted alkoxy. In some embodiments, R₁₄ is substituted alkoxy. In some embodiments, R₁₄ is unsubstituted aryl. In some embodiments, R₁₄ is substituted aryl. In some embodiments, R₁₄ is unsubstituted C₃-C₁₀heterocyclyl. In some embodiments, R₁₄ is substituted C₃-C₁₀heterocyclyl. In some embodiments, R₁₄ is unsubstituted heteroaryl. In some embodiments, R₁₄ is unsubstituted C₃-C₁₀cycloalkyl. In some embodiments, R₁₄ is substituted C₃-C₁₀cycloalkyl. In some embodiments, R₁₄ is —OPO₃WY. In some embodiments, R₁₄ is —OCH₂PO₄WY. In some embodiments, R₁₄ is —OCH₂PO₄Z. In some embodiments, R₁₄ is —OPO₃Z.

In some embodiments, R₁₅ is hydrogen. In some embodiments, R₁₅ is hydroxyl. In some embodiments, R₁₅ is carboxaldehyde. In some embodiments, R₁₅ is unsubstituted amine. In some embodiments, R₁₅ is substituted amine. In some embodiments, R₁₅ is unsubstituted C₁-C₁₀ alkyl. In some embodiments, R₁₅ is substituted C₁-C₁₀ alkyl. In some embodiments, R₁₅ is unsubstituted C₂-C₁₀ alkynyl. In some embodiments, R₁₅ is substituted C₂-C₁₀ alkynyl. In some embodiments, R₁₅ is unsubstituted C₂-C₁₀ alkenyl. In some embodiments, R₁₅ is substituted C₂-C₁₀ alkenyl. In some embodiments, R₁₅ is carboxyl. In some embodiments, R₁₅ is unsubstituted carbohydrate. In some embodiments, R₁₅ is substituted carbohydrate. In some embodiments, R₁₅ is unsubstituted ester. In some embodiments, R₁₅ is substituted ester. In some embodiments, R₁₅ is unsubstituted acyloxy. In some embodiments, R₁₅ is substituted acyloxy. In some embodiments, R₁₃ is nitro. In some embodiments, R₁₃ is halogen. In some embodiments, R₁₃ is unsubstituted C₁-C₁₀ aliphatic acyl. In some embodiments, R₁₅ is substituted C₁-C₁₀ aliphatic acyl. In some embodiments, R₁₅ is unsubstituted C₆-C₁₀ aromatic acyl. In some embodiments, R₁₅ is substituted C₆-C₁₀ aromatic acyl. In some embodiments, R₁₅ is unsubstituted C₆-C₁₀ aralkyl acyl. In some embodiments, R₁₅ is substituted C₆-C₁₀ aralkyl acyl. In some embodiments, R₁₅ is unsubstituted C₆-C₁₀ alkylaryl acyl. In some embodiments, R₁₅ is substituted C₆-C₁₀ alkylaryl acyl. In some embodiments, R₁₅ is unsubstituted alkoxy. In some embodiments, R₁₅ is substituted alkoxy. In some embodiments, R₁₅ is unsubstituted aryl. In some embodiments, R₁₅ is substituted aryl. In some embodiments, R₁₅ is unsubstituted C₃-C₁₀heterocyclyl. In some embodiments, R₁₅ is substituted C₃-C₁₀heterocyclyl. In some embodiments, R₁₅ is unsubstituted heteroaryl, In some embodiments, R₁₅ is unsubstituted C₃-C₁₀cycloalkyl. In some embodiments, R₁₅ is substituted C₃-C₁₀cycloalkyl. In some embodiments, R₁₅ is —OPO₃WY. In some embodiments, R₁₅ is —OCH₂PO₄WY. In some embodiments, R₁₅ is —OCH₂PO₄Z. In some embodiments, R₁₅ is —OPO₃Z.

In some embodiments, R₁₆ is hydrogen. In some embodiments, R₁₆ is unsubstituted C₁-C₁₀ alkyl. In some embodiments, R₁₆ is substituted C₁-C₁₀ alkyl. In some embodiments, R₁₆ is unsubstituted C₂-C₁₀ alkynyl. In some embodiments, R₁₆ is substituted C₂-C₁₀ alkynyl. In some embodiments, R₁₆ is unsubstituted C₂-C₁₀ alkenyl. In some embodiments, R₁₆ is substituted C₂-C₁₀ alkenyl. In some embodiments, R₁₆ is unsubstituted carbohydrate. In some embodiments, R₁₆ is substituted carbohydrate. In some embodiments, R₁₆ is unsubstituted C₁-C₁₀ aliphatic acyl. In some embodiments, R₁₆ is substituted C₁-C₁₀ aliphatic acyl. In some embodiments, R₁₆ is unsubstituted C₆-C₁₀ aromatic acyl. In some embodiments, R₁₆ is substituted C₆-C₁₀ aromatic acyl. In some embodiments, R₁₆ is unsubstituted C₆-C₁₀ aralkyl acyl. In some embodiments, R₁₆ is substituted C₆-C₁₀ aralkyl acyl. In some embodiments, R₁₆ is unsubstituted C₆-C₁₀ alkylaryl acyl. In some embodiments, R₁₆ is substituted C₆-C₁₀ alkylaryl acyl. In some embodiments, R₁₆ is unsubstituted aryl. In some embodiments, R₁₆ is substituted aryl. In some embodiments, R₁₆ is unsubstituted C₃-C₁₀heterocyclyl. In some embodiments, R₁₆ is substituted C₃-C₁₀ heterocyclyl. In some embodiments, R₁₆ is unsubstituted heteroaryl. In some embodiments, R₁₆ is substituted heteroaryl. In some embodiments, R₁₆ is unsubstituted C₃-C₁₀cycloalkyl. In some embodiments, R₁₆ is substituted C₃-C₁₀cycloalkyl. In some embodiments, R₁₆ is —PO₃WY. In some embodiments, R₁₆ is —CH₂PO₄WY. In some embodiments, R₁₆ is —CH₂PO₄Z. In some embodiments, R₁₆ is —PO₃Z.

In some embodiments, R₁₈ is hydrogen. In some embodiments, R₁₈ is hydroxy. In some embodiments, R₁₈ is carboxaldehyde. In some embodiments, R₁₈ is unsubstituted amine. In some embodiments, R₁₈ is substituted amine. In some embodiments, R₁₈ is unsubstituted C₁-C₁₀ alkyl. In some embodiments, R₁₈ is unsubstituted C₂-C₁₀ alkynyl. In some embodiments, R₁₈ is substituted C₂-C₁₀ alkynyl. In some embodiments, R₁₈ is unsubstituted C₂-C₁₀ alkenyl. In some embodiments, R₁₈ is substituted C₂-C₁₀ alkenyl. In some embodiments, R₁₈ is carboxyl. In some embodiments, R₁₈ is unsubstituted carbohydrate. In some embodiments, R₁₈ is substituted carbohydrate. In some embodiments, R₁₈ is substituted carbohydrate. In some embodiments, R₁₈ is unsubstituted ester. In some embodiments, R₁₈ is substituted ester. In some embodiments, R₁₈ is unsubstituted acyloxy. In some embodiments, R₁₈ is substituted acyloxy. In some embodiments, R₁₈ is nitro. In some embodiments, R₁₈ is halogen. In some embodiments, R₁₈ is unsubstituted C₁-C₁₀ aliphatic acyl. In some embodiments, R₁₈ is substituted C₁-C₁₀ aliphatic acyl. In some embodiments, R₁₈ is unsubstituted C₆-C₁₀ aromatic acyl. In some embodiments, R₁₈ is substituted C₆-C₁₀ aromatic acyl. In some embodiments, R₁₈ is unsubstituted C₆-C₁₀ aralkyl acyl. In some embodiments, R₁₈ is substituted C₆-C₁₀ aralkyl acyl. In some embodiments, R₁₈ is unsubstituted C₆-C₁₀ alkylaryl acyl. In some embodiments, R₁₈ is substituted C₆-C₁₀ alkylaryl acyl. In some embodiments, R₁₈ is unsubstituted alkoxy. In some embodiments, R₁₈ is substituted alkoxy. In some embodiments, R₁₈ is unsubstituted aryl. In some embodiments, R₁₈ is substituted aryl. In some embodiments, R₁₈ is unsubstituted C₃-C₁₀heterocyclyl. In some embodiments, R₁₈ is substituted C₃-C₁₀heterocyclyl. In some embodiments, R₁₈ is unsubstituted heteroaryl. In some embodiments, R₁₈ is substituted heteroaryl. In some embodiments, R₁₈ is unsubstituted C₃-C₁₀cycloalkyl. In some embodiments, R₁₈ is substituted C₃-C₁₀cycloalkyl. In some embodiments, R₁₈ is —OPO₃WY. In some embodiments, R₁₈ is —OCH₂PO₄WY. In some embodiments, R₁₈ is —OCH₂PO₄Z. In some embodiments, R₁₈ is —OPO₃Z.

In some embodiments, R₁₉ is hydrogen. In some embodiments, R₁₉ is unsubstituted C₁-C₁₀ alkyl. In some embodiments, R₁₉ is substituted C₁-C₁₀ alkyl. In some embodiments, R₁₉ is unsubstituted C₂-C₁₀ alkynyl. In some embodiments, R₁₉ is substituted C₂-C₁₀ alkynyl. In some embodiments, R₁₉ is unsubstituted C₂-C₁₀ alkenyl. In some embodiments, R₁₉ is substituted C₂-C₁₀ alkenyl. In some embodiments, R₁₉ is unsubstituted carbohydrate. In some embodiments, R₁₉ is substituted carbohydrate. In some embodiments, R₁₉ is unsubstituted C₁-C₁₀ aliphatic acyl. In some embodiments, R₁₉ is substituted C₁-C₁₀ aliphatic acyl. In some embodiments, R₁₉ is unsubstituted C₆-C₁₀ aromatic acyl. In some embodiments, R₁₉ is substituted C₆-C₁₀ aromatic acyl. In some embodiments, R₁₉ is unsubstituted C₆-C₁₀ aralkyl acyl. In some embodiments, R₁₉ is substituted C₆-C₁₀ aralkyl acyl. In some embodiments, R₁₉ is unsubstituted C₆-C₁₀ alkylaryl acyl. In some embodiments, R₁₉ is substituted C₆-C₁₀ alkylaryl acyl. In some embodiments, R₁₉ is unsubstituted aryl. In some embodiments, R₁₉ is substituted aryl. In some embodiments, R₁₉ is unsubstituted C₃-C₁₀heterocyclyl. In some embodiments, R₁₉ is substituted C₃-C₁₀ heterocyclyl. In some embodiments, R₁₉ is unsubstituted heteroaryl. In some embodiments, R₁₉ is substituted heteroaryl. In some embodiments, R₁₉ is unsubstituted C₃-C₁₀cycloalkyl. In some embodiments, R₁₉ is substituted C₃-C₁₀cycloalkyl. In some embodiments, R₁₉ is —PO₃WY. In some embodiments, R₁₉ is —CH₂PO₄WY. In some embodiments, R₁₉ is —CH₂PO₄Z. In some embodiments, R₁₉ is —PO₃Z.

In some embodiments, s is an integer of 0. In some embodiments, s is an integer of 1. In some embodiments, s is an integer of 2. In some embodiments, s is an integer of 3.

In various embodiments, W is hydrogen. In various embodiments, W is unsubstituted methyl. In various embodiments, W is substituted methyl. In various embodiments, W is unsubstituted ethyl. In various embodiments, W is substituted ethyl. In various embodiments, W is unsubstituted alkyl. In various embodiments, W is substituted alkyl. In various embodiments, W is unsubstituted carbohydrate. In various embodiments, W is substituted carbohydrate. In various embodiments, W is potassium. In various embodiments, W is sodium. In various embodiments, W is lithium.

In various embodiments, Y is hydrogen. In various embodiments, Y is unsubstituted methyl. In various embodiments, Y is substituted methyl. In various embodiments, Y is unsubstituted ethyl. In various embodiments, Y is substituted ethyl. In various embodiments, Y is unsubstituted alkyl. In various embodiments, Y is substituted alkyl. In various embodiments, Y is unsubstituted carbohydrate. In various embodiments, Y is substituted carbohydrate. In various embodiments, Y is potassium. In various embodiments, Y is sodium. In various embodiments, Y is lithium.

In various embodiments, Z is calcium. In various embodiments, Z is magnesium. In various embodiments, Z is iron.

In some embodiments of the invention, the pyrone analog of Formula VI is of Formula XXXII or Formula XXXIII or a pharmaceutically/veterinarily acceptable salt thereof:

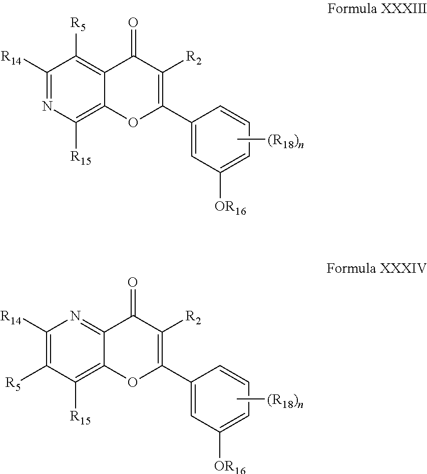

wherein R₁₆ is hydrogen, —PO₃WY, —CH₂PO₄WY, —CH₂PO₄Z or —PO₃Z;

wherein each instance of R₁₈ is independently hydrogen, hydroxyl, carboxaldehyde, amine, C₁-C₁₀ alkyl, C₂-C₁₀ alkynyl, C₂-C₁₀ alkenyl, carboxyl, carbohydrate, ester, acyloxy, nitro, halogen, C₁-C₁₀ aliphatic acyl, C₆-C₁₀ aromatic acyl, C₆-C₁₀ aralkyl acyl, C₆-C₁₀ alkylaryl acyl, alkoxy, alkyl, phosphate, aryl, heteroaryl, C₃-C₁₀ heterocyclic, C₃-C₁₀cycloalkyl, —OPO₃WY, —OCH₂PO₄WY, —OCH₂PO₄Z or —OPO₃Z; and

n is an integer of 0, 1, 2, 3, or 4.

In some embodiments of the invention, R₁₆ is —PO₃WY, —CH₂PO₄WY, —CH₂PO₄Z or —PO₃Z, or at least one of R₁₈ is —OPO₃WY, —OCH₂PO₄WY, —OCH₂PO₄Z or —OPO₃Z. In some embodiments, R₁₆ is —PO₃WY, —CH₂PO₄WY, —CH₂PO₄Z or —PO₃Z and at least one of R₁₈ is —OPO₃WY, —OCH₂PO₄WY, —OCH₂PO₄Z or —OPO₃Z.

In some embodiments of the invention, the pyrone analog of Formula VI is of Formula XXXIV or a pharmaceutically/veterinarily acceptable salt thereof:

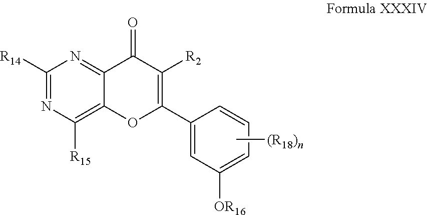

wherein R₁₆ is hydrogen, —PO₃WY, —CH₂PO₄WY, —CH₂PO₄Z or —PO₃Z;

wherein each instance of R₁₈ is independently hydrogen, hydroxyl, carboxaldehyde, amine, C₁-C₁₀ alkyl, C₂-C₁₀ alkynyl, C₂-C₁₀ alkenyl, carboxyl, carbohydrate, ester, acyloxy, nitro, halogen, C₁-C₁₀ aliphatic acyl, C₆-C₁₀ aromatic acyl, C₆-C₁₀ aralkyl acyl, C₆-C₁₀ alkylaryl acyl, alkoxy, alkyl, phosphate, aryl, heteroaryl, C₃-C₁₀ heterocyclic, C₃-C₁₀cycloalkyl, —OPO₃WY, —OCH₂PO₄WY, —OCH₂PO₄Z or —OPO₃Z; and

n is an integer of 0, 1, 2, 3, or 4.

In some embodiments of the invention, R₁₆ is —PO₃WY, —CH₂PO₄WY, —CH₂PO₄Z or —PO₃Z, or at least one of R₁₈ is —OPO₃WY, —OCH₂PO₄WY, —OCH₂PO₄Z or —OPO₃Z. In some embodiments, R₁₆ is —PO₃WY, —CH₂PO₄WY, —CH₂PO₄Z or —PO₃Z and at least one of R₁₈ is —OPO₃WY, —OCH₂PO₄WY, —OCH₂PO₄Z or —OPO₃Z.

Each instance of R₁, R₂, R₃, R₄, R₅, R₆, R₇, R₈, R₉, R₁₀, R₁₁, R₁₂, R₁₃, R₁₄, R₁₅, R₁₆, R₁₇, R₁₈, R₁₉, R₂₀, R₂₁, W, Y and Z disclosed may be used in any combination in any of the Formulae I, II, III, IV, V, VI, VII, VIII, IX, X, XI, XII, XIII, XIV, XV, XVI, XVII, XVIII, XIX, XX, XXI, XXII, XXIII, XXIV, XXV, XXVI, XXVII, XXVIII, XXIX, XXX, XXXI, XXXII, XXXIII, and XXXIV.

### III. METHODS OF SYNTHESIS OF THE COMPOUNDS OF THE INVENTION

The compounds of the invention may be synthesized via several approaches, and the particular synthesis route is chosen depending on the substituent pattern of the desired compound. The routes are illustrated with selected members of the class of compounds of the invention, but can be used for other compounds described herein. Note that Scheme 1 is found at paragraph 222.

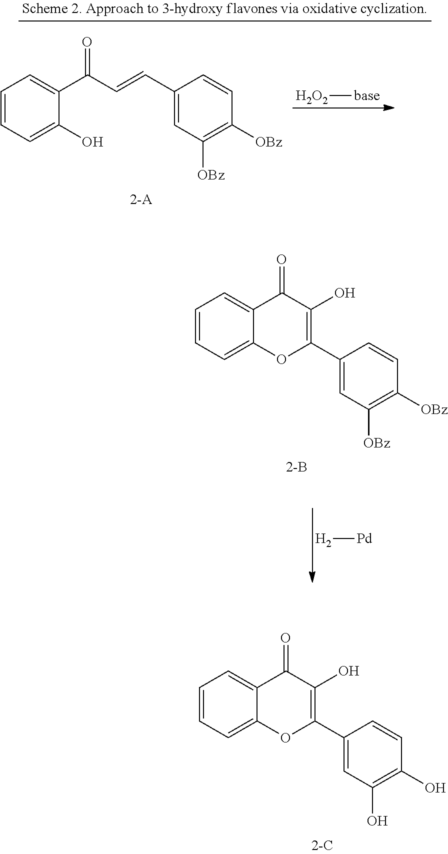

In this synthetic route, a benzyl protected ortho-hydroxyphenyl styrylketone 2-A is cyclized to yield a 3-hydroxy flavone using hydrogen peroxide and base. Deprotection via treatment with hydrogen and palladium catalysts yields a 3′-,4′-dihydroxyphenyl 3-hydroxy flavone 2-C. (See Chemical Research in Toxicology, 17(6), 795-804 (2004).)

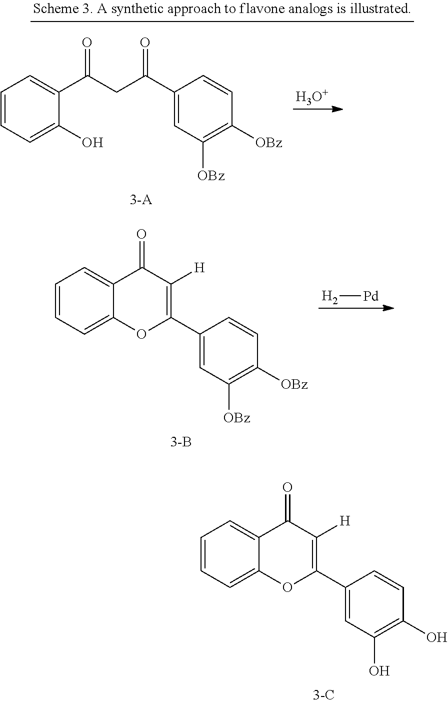

Another approach to compounds of the invention is via acid catalyzed cyclization of an 1-ortho-hydroxyphenyl-3-aryl-1, 3, propanedione 3-A to yield a flavone analog 3-C, as is illustrated in Scheme 3. Benzyl protection of the dihydroxy substituents on the 3-aryl moiety is shown, but methyl ether protection may also be used. After cyclization is accomplished, the protecting groups are removed from compound 3-B. In the case of benzyl protection, treatment with hydrogen in the presence of a palladium is used. If methyl ether protection is used, treatment of the cyclized protected species analogous to compound 3-B with trimethylsilyl iodide will produce the final product 3-C. (See Chemical Research in Toxicology, 17(6), 795-804 (2004).)

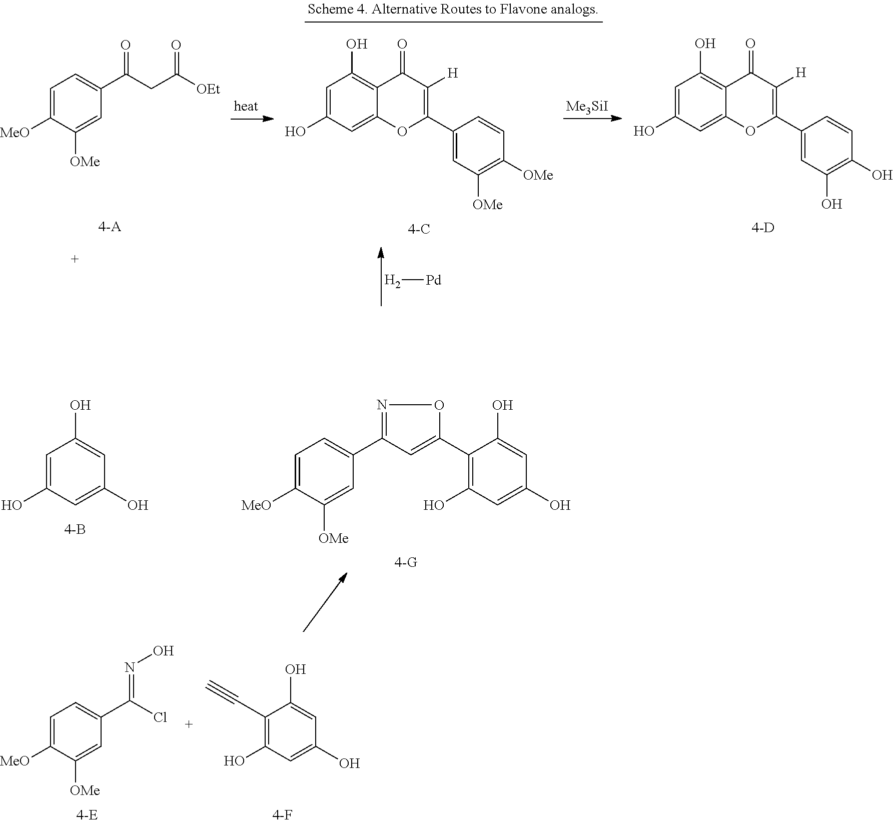

Alternative cyclizations to yield flavone analogs may also be used and is illustrated in Scheme 4. Beta-ketoester 4-A is cyclized with 1, 3, 5, trihydroxybenzene using heat to yield the protected flavone 4-C. Deprotection with trimethylsilyl iodide yields a flavone 4-D. The same intermediate 4-C is obtained by reacting the acetylenic trihydroxybenzene 4-F with compound 4-E to yield the tricyclic intermediate 4-G. Reduction of 4-G with hydrogen over palladium yields 4-C, which can be converted to the flavone 4-D as before. (See Faming Zhuanli Shenquig Gongkai Shuomingshu 1666987 (2005), and Acta Chemica Scandinavica 49(7), 524-9 (1995).)

### IV. EXEMPLARY COMPOUNDS OF THE INVENTION

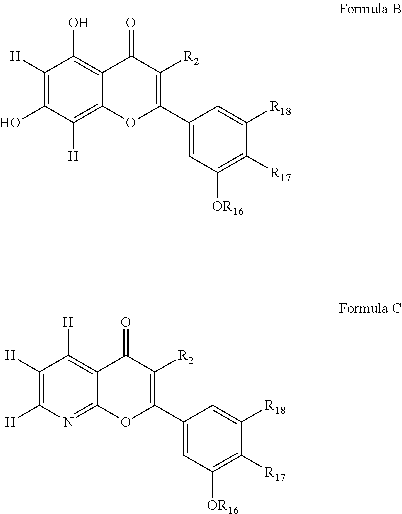

Key: Substituents described in Table 1 of exemplary compounds are abbreviated as shown herein.

| | |
|---|---|
| E = —O-glucoronide | G = -glucoronide |
| J = —PO₃T | K = —OPO₃T |
| V = —CH₂OPO₃T | L = —OCH₂OPO₃T |

TABLE A
Exemplary formulas of T as described in the Exemplary compounds
of Formula B or Formula C as disclosed in Table 1.

| Subclass # | T | Subclass # | T |
|---|---|---|---|
| S-1 | H, H | S-2 | H, K |
| S-3 | K, K | S-4 | H, Li |
| S-5 | Li, Li | S-6 | H, Na |
| S-7 | Na, Na | S-8 | H, Me |
| S-9 | Me, Me | S-10 | H, ethyl |
| S-11 | ethyl, ethyl | S-12 | H, glucose |
| S-13 | Glucose, glucose | S-14 | Ca |
| S-15 | Mg | S-16 | Fe(II) |
| S-17 | Zn | | |

TABLE 1
Exemplary compounds of Formula B or Formula C. Each subclass in Table 1, utilizes each subclass of “T” as described in Table A.

| Sub- | | | | | | | | | | | | | | | | | | | | | | | | | |
|---|---|---|---|---|---|---|---|---|---|---|---|---|---|---|---|---|---|---|---|---|---|---|---|---|---|
| class # | R₂ | | | | | R₁₆ | | | | | R₁₇ | | | | | | R₁₈ | | | | | | | | |
| 1 | H | OH | E | K | L | H | Me | G | J | V | H | E | K | L | OMe | Me | H | E | K | L | Me | OH | NH₂ | CN | F |
| 2 | x | | | | | x | | | | | x | | | | | | x | | | | | | | | |
| 3 | x | | | | | x | | | | | x | | | | | | | x | | | | | | | |
| 4 | x | | | | | x | | | | | x | | | | | | | | x | | | | | | |
| 5 | x | | | | | x | | | | | x | | | | | | | | | x | | | | | |
| 6 | x | | | | | x | | | | | x | | | | | | | | | | x | | | | |
| 7 | x | | | | | x | | | | | x | | | | | | | | | | | x | | | |
| 8 | x | | | | | x | | | | | x | | | | | | | | | | | | x | | |
| 9 | x | | | | | x | | | | | x | | | | | | | | | | | | | x | |
| 10 | x | | | | | x | | | | | x | | | | | | | | | | | | | | x |
| 11 | x | | | | | x | | | | | | x | | | | | x | | | | | | | | |
| 12 | x | | | | | x | | | | | | x | | | | | | x | | | | | | | |
| 13 | x | | | | | x | | | | | | x | | | | | | | x | | | | | | |
| 14 | x | | | | | x | | | | | | x | | | | | | | | x | | | | | |
| 15 | x | | | | | x | | | | | | x | | | | | | | | | x | | | | |
| 16 | x | | | | | x | | | | | | x | | | | | | | | | | x | | | |
| 17 | x | | | | | x | | | | | | x | | | | | | | | | | | x | | |
| 18 | x | | | | | x | | | | | | x | | | | | | | | | | | | x | |
| 19 | x | | | | | x | | | | | | x | | | | | | | | | | | | | x |
| 20 | x | | | | | x | | | | | | | x | | | | x | | | | | | | | |
| 21 | x | | | | | x | | | | | | | x | | | | | x | | | | | | | |
| 22 | x | | | | | x | | | | | | | x | | | | | | x | | | | | | |
| 23 | x | | | | | x | | | | | | | x | | | | | | | x | | | | | |
| 24 | x | | | | | x | | | | | | | x | | | | | | | | x | | | | |
| 25 | x | | | | | x | | | | | | | x | | | | | | | | | x | | | |
| 26 | x | | | | | x | | | | | | | x | | | | | | | | | | | | |
| 27 | x | | | | | x | | | | | | | x | | | | | | | | | | x | | |
| 28 | x | | | | | x | | | | | | | x | | | | | | | | | | | x | |
| 29 | x | | | | | x | | | | | | | x | | | | | | | | | | | | x |
| 30 | x | | | | | x | | | | | | | | x | | | x | | | | | | | | |
| 31 | x | | | | | x | | | | | | | | x | | | | x | | | | | | | |
| 32 | x | | | | | x | | | | | | | | x | | | | | x | | | | | | |
| 33 | x | | | | | x | | | | | | | | x | | | | | | x | | | | | |
| 34 | x | | | | | x | | | | | | | | x | | | | | | | x | | | | |
| 35 | x | | | | | x | | | | | | | | x | | | | | | | | x | | | |
| 36 | x | | | | | x | | | | | | | | x | | | | | | | | | x | | |
| 37 | x | | | | | x | | | | | | | | x | | | | | | | | | | x | |
| 38 | x | | | | | x | | | | | | | | x | | | | | | | | | | | x |
| 39 | x | | | | | x | | | | | | | | | x | | x | | | | | | | | |
| 40 | x | | | | | x | | | | | | | | | x | | | x | | | | | | | |
| 41 | x | | | | | x | | | | | | | | | x | | | | x | | | | | | |
| 42 | x | | | | | x | | | | | | | | | x | | | | | x | | | | | |
| 43 | x | | | | | x | | | | | | | | | x | | | | | | x | | | | |
| 44 | x | | | | | x | | | | | | | | | x | | | | | | | x | | | |
| 45 | x | | | | | x | | | | | | | | | x | | | | | | | | x | | |
| 46 | x | | | | | x | | | | | | | | | x | | | | | | | | | x | |
| 47 | x | | | | | x | | | | | | | | | x | | | | | | | | | | x |
| 48 | x | | | | | x | | | | | | | | | | x | x | | | | | | | | |
| 49 | x | | | | | x | | | | | | | | | | x | | x | | | | | | | |
| 50 | x | | | | | x | | | | | | | | | | x | | | x | | | | | | |
| 51 | x | | | | | x | | | | | | | | | | x | | | | x | | | | | |
| 52 | x | | | | | x | | | | | | | | | | x | | | | | x | | | | |
| 53 | x | | | | | x | | | | | | | | | | x | | | | | | x | | | |
| 54 | x | | | | | x | | | | | | | | | | x | | | | | | | x | | |
| 55 | x | | | | | x | | | | | | | | | | x | | | | | | | | x | |
| 56 | x | | | | | x | | | | | | | | | | x | | | | | | | | | x |
| 57 | x | | | | | | x | | | | x | | | | | | x | | | | | | | | |
| 58 | x | | | | | | x | | | | x | | | | | | | x | | | | | | | |
| 59 | x | | | | | | x | | | | x | | | | | | | | x | | | | | | |
| 60 | x | | | | | | x | | | | x | | | | | | | | | x | | | | | |
| 61 | x | | | | | | x | | | | x | | | | | | | | | | x | | | | |
| 62 | x | | | | | | x | | | | x | | | | | | | | | | | x | | | |
| 63 | x | | | | | | x | | | | x | | | | | | | | | | | | x | | |
| 64 | x | | | | | | x | | | | x | | | | | | | | | | | | | x | |
| 65 | x | | | | | | x | | | | x | | | | | | | | | | | | | | x |
| 66 | x | | | | | | x | | | | | x | | | | | x | | | | | | | | |
| 67 | x | | | | | | x | | | | | x | | | | | | x | | | | | | | |
| 68 | x | | | | | | x | | | | | x | | | | | | | x | | | | | | |
| 69 | x | | | | | | x | | | | | x | | | | | | | | x | | | | | |
| 70 | x | | | | | | x | | | | | x | | | | | | | | | x | | | | |
| 71 | x | | | | | | x | | | | | x | | | | | | | | | | x | | | |
| 72 | x | | | | | | x | | | | | x | | | | | | | | | | | x | | |
| 73 | x | | | | | | x | | | | | x | | | | | | | | | | | | x | |
| 74 | x | | | | | | x | | | | | x | | | | | | | | | | | | | x |
| 75 | x | | | | | | x | | | | | | x | | | | x | | | | | | | | |
| 76 | x | | | | | | x | | | | | | x | | | | | x | | | | | | | |
| 77 | x | | | | | | x | | | | | | x | | | | | | x | | | | | | |
| 78 | x | | | | | | x | | | | | | x | | | | | | | x | | | | | |
| 79 | x | | | | | | x | | | | | | x | | | | | | | | x | | | | |
| 80 | x | | | | | | x | | | | | | x | | | | | | | | | x | | | |
| 81 | x | | | | | | x | | | | | | x | | | | | | | | | | | | |
| 82 | x | | | | | | x | | | | | | x | | | | | | | | | | x | | |
| 83 | x | | | | | | x | | | | | | x | | | | | | | | | | | x | |
| 84 | x | | | | | | x | | | | | | x | | | | | | | | | | | | x |
| 85 | x | | | | | | x | | | | | | | x | | | x | | | | | | | | |
| 86 | x | | | | | | x | | | | | | | x | | | | x | | | | | | | |
| 87 | x | | | | | | x | | | | | | | x | | | | | x | | | | | | |
| 88 | x | | | | | | x | | | | | | | x | | | | | | x | | | | | |
| 89 | x | | | | | | x | | | | | | | x | | | | | | | x | | | | |
| 90 | x | | | | | | x | | | | | | | x | | | | | | | | x | | | |
| 91 | x | | | | | | x | | | | | | | x | | | | | | | | | x | | |
| 92 | x | | | | | | x | | | | | | | x | | | | | | | | | | x | |
| 93 | x | | | | | | x | | | | | | | x | | | | | | | | | | | x |
| 94 | x | | | | | | x | | | | | | | | x | | x | | | | | | | | |
| 95 | x | | | | | | x | | | | | | | | x | | | x | | | | | | | |
| 96 | x | | | | | | x | | | | | | | | x | | | | x | | | | | | |
| 97 | x | | | | | | x | | | | | | | | x | | | | | x | | | | | |
| 98 | x | | | | | | x | | | | | | | | x | | | | | | x | | | | |
| 99 | x | | | | | | x | | | | | | | | x | | | | | | | x | | | |
| 100 | x | | | | | | x | | | | | | | | x | | | | | | | | x | | |
| 101 | x | | | | | | x | | | | | | | | x | | | | | | | | | x | |
| 102 | x | | | | | | x | | | | | | | | x | | | | | | | | | | x |
| 103 | x | | | | | | x | | | | | | | | | x | x | | | | | | | | |
| 104 | x | | | | | | x | | | | | | | | | x | | x | | | | | | | |
| 105 | x | | | | | | x | | | | | | | | | x | | | x | | | | | | |
| 106 | x | | | | | | x | | | | | | | | | x | | | | x | | | | | |
| 107 | x | | | | | | x | | | | | | | | | x | | | | | x | | | | |
| 108 | x | | | | | | x | | | | | | | | | x | | | | | | x | | | |
| 109 | x | | | | | | x | | | | | | | | | x | | | | | | | x | | |
| 110 | x | | | | | | x | | | | | | | | | x | | | | | | | | x | |
| 111 | x | | | | | | x | | | | | | | | | x | | | | | | | | | x |
| 112 | x | | | | | | | x | | | x | | | | | | x | | | | | | | | |
| 113 | x | | | | | | | x | | | x | | | | | | | x | | | | | | | |
| 114 | x | | | | | | | x | | | x | | | | | | | | x | | | | | | |
| 115 | x | | | | | | | x | | | x | | | | | | | | | x | | | | | |
| 116 | x | | | | | | | x | | | x | | | | | | | | | | x | | | | |
| 117 | x | | | | | | | x | | | x | | | | | | | | | | | x | | | |
| 118 | x | | | | | | | x | | | x | | | | | | | | | | | | x | | |
| 119 | x | | | | | | | x | | | x | | | | | | | | | | | | | x | |
| 120 | x | | | | | | | x | | | x | | | | | | | | | | | | | | x |
| 121 | x | | | | | | | x | | | | x | | | | | x | | | | | | | | |
| 122 | x | | | | | | | x | | | | x | | | | | | x | | | | | | | |
| 123 | x | | | | | | | x | | | | x | | | | | | | x | | | | | | |
| 124 | x | | | | | | | x | | | | x | | | | | | | | x | | | | | |
| 125 | x | | | | | | | x | | | | x | | | | | | | | | x | | | | |
| 126 | x | | | | | | | x | | | | x | | | | | | | | | | x | | | |
| 127 | x | | | | | | | x | | | | x | | | | | | | | | | | x | | |
| 128 | x | | | | | | | x | | | | x | | | | | | | | | | | | x | |
| 129 | x | | | | | | | x | | | | x | | | | | | | | | | | | | x |
| 130 | x | | | | | | | x | | | | | x | | | | x | | | | | | | | |
| 131 | x | | | | | | | x | | | | | x | | | | | x | | | | | | | |
| 132 | x | | | | | | | x | | | | | x | | | | | | x | | | | | | |
| 133 | x | | | | | | | x | | | | | x | | | | | | | x | | | | | |
| 134 | x | | | | | | | x | | | | | x | | | | | | | | x | | | | |
| 135 | x | | | | | | | x | | | | | x | | | | | | | | | x | | | |
| 136 | x | | | | | | | x | | | | | x | | | | | | | | | | | | |
| 137 | x | | | | | | | x | | | | | x | | | | | | | | | | x | | |
| 138 | x | | | | | | | x | | | | | x | | | | | | | | | | | x | |
| 139 | x | | | | | | | x | | | | | x | | | | | | | | | | | | x |
| 140 | x | | | | | | | x | | | | | | x | | | x | | | | | | | | |
| 141 | x | | | | | | | x | | | | | | x | | | | x | | | | | | | |
| 142 | x | | | | | | | x | | | | | | x | | | | | x | | | | | | |
| 143 | x | | | | | | | x | | | | | | x | | | | | | x | | | | | |
| 144 | x | | | | | | | x | | | | | | x | | | | | | | x | | | | |
| 145 | x | | | | | | | x | | | | | | x | | | | | | | | x | | | |
| 146 | x | | | | | | | x | | | | | | x | | | | | | | | | x | | |
| 147 | x | | | | | | | x | | | | | | x | | | | | | | | | | x | |
| 148 | x | | | | | | | x | | | | | | x | | | | | | | | | | | x |
| 149 | x | | | | | | | x | | | | | | | x | | x | | | | | | | | |
| 150 | x | | | | | | | x | | | | | | | x | | | x | | | | | | | |
| 151 | x | | | | | | | x | | | | | | | x | | | | x | | | | | | |
| 152 | x | | | | | | | x | | | | | | | x | | | | | x | | | | | |
| 153 | x | | | | | | | x | | | | | | | x | | | | | | x | | | | |
| 154 | x | | | | | | | x | | | | | | | x | | | | | | | x | | | |
| 155 | x | | | | | | | x | | | | | | | x | | | | | | | | x | | |
| 156 | x | | | | | | | x | | | | | | | x | | | | | | | | | x | |
| 157 | x | | | | | | | x | | | | | | | x | | | | | | | | | | x |
| 158 | x | | | | | | | x | | | | | | | | x | x | | | | | | | | |
| 159 | x | | | | | | | x | | | | | | | | x | | x | | | | | | | |
| 160 | x | | | | | | | x | | | | | | | | x | | | x | | | | | | |
| 161 | x | | | | | | | x | | | | | | | | x | | | | x | | | | | |
| 162 | x | | | | | | | x | | | | | | | | x | | | | | x | | | | |
| 163 | x | | | | | | | x | | | | | | | | x | | | | | | x | | | |
| 164 | x | | | | | | | x | | | | | | | | x | | | | | | | x | | |
| 165 | x | | | | | | | x | | | | | | | | x | | | | | | | | x | |
| 166 | x | | | | | | | x | | | | | | | | x | | | | | | | | | x |
| 167 | x | | | | | | | | x | | x | | | | | | x | | | | | | | | |
| 168 | x | | | | | | | | x | | x | | | | | | | x | | | | | | | |
| 169 | x | | | | | | | | x | | x | | | | | | | | x | | | | | | |
| 170 | x | | | | | | | | x | | x | | | | | | | | | x | | | | | |
| 171 | x | | | | | | | | x | | x | | | | | | | | | | x | | | | |
| 172 | x | | | | | | | | x | | x | | | | | | | | | | | x | | | |
| 173 | x | | | | | | | | x | | x | | | | | | | | | | | | x | | |
| 174 | x | | | | | | | | x | | x | | | | | | | | | | | | | x | |
| 175 | x | | | | | | | | x | | x | | | | | | | | | | | | | | x |
| 176 | x | | | | | | | | x | | | x | | | | | x | | | | | | | | |
| 177 | x | | | | | | | | x | | | x | | | | | | x | | | | | | | |
| 178 | x | | | | | | | | x | | | x | | | | | | | x | | | | | | |
| 179 | x | | | | | | | | x | | | x | | | | | | | | x | | | | | |
| 180 | x | | | | | | | | x | | | x | | | | | | | | | x | | | | |
| 181 | x | | | | | | | | x | | | x | | | | | | | | | | x | | | |
| 182 | x | | | | | | | | x | | | x | | | | | | | | | | | x | | |
| 183 | x | | | | | | | | x | | | x | | | | | | | | | | | | x | |
| 184 | x | | | | | | | | x | | | x | | | | | | | | | | | | | x |
| 185 | x | | | | | | | | x | | | | x | | | | x | | | | | | | | |
| 186 | x | | | | | | | | x | | | | x | | | | | x | | | | | | | |
| 187 | x | | | | | | | | x | | | | x | | | | | | x | | | | | | |
| 188 | x | | | | | | | | x | | | | x | | | | | | | x | | | | | |
| 189 | x | | | | | | | | x | | | | x | | | | | | | | x | | | | |
| 190 | x | | | | | | | | x | | | | x | | | | | | | | | x | | | |
| 191 | x | | | | | | | | x | | | | x | | | | | | | | | | | | |
| 192 | x | | | | | | | | x | | | | x | | | | | | | | | | x | | |
| 193 | x | | | | | | | | x | | | | x | | | | | | | | | | | x | |
| 194 | x | | | | | | | | x | | | | x | | | | | | | | | | | | x |
| 195 | x | | | | | | | | x | | | | | x | | | x | | | | | | | | |
| 196 | x | | | | | | | | x | | | | | x | | | | x | | | | | | | |
| 197 | x | | | | | | | | x | | | | | x | | | | | x | | | | | | |
| 198 | x | | | | | | | | x | | | | | x | | | | | | x | | | | | |
| 199 | x | | | | | | | | x | | | | | x | | | | | | | x | | | | |
| 200 | x | | | | | | | | x | | | | | x | | | | | | | | x | | | |
| 201 | x | | | | | | | | x | | | | | x | | | | | | | | | x | | |
| 202 | x | | | | | | | | x | | | | | x | | | | | | | | | | x | |
| 203 | x | | | | | | | | x | | | | | x | | | | | | | | | | | x |
| 204 | x | | | | | | | | x | | | | | | x | | x | | | | | | | | |
| 205 | x | | | | | | | | x | | | | | | x | | | x | | | | | | | |
| 206 | x | | | | | | | | x | | | | | | x | | | | x | | | | | | |
| 207 | x | | | | | | | | x | | | | | | x | | | | | x | | | | | |
| 208 | x | | | | | | | | x | | | | | | x | | | | | | x | | | | |
| 209 | x | | | | | | | | x | | | | | | x | | | | | | | x | | | |
| 210 | x | | | | | | | | x | | | | | | x | | | | | | | | x | | |
| 211 | x | | | | | | | | x | | | | | | x | | | | | | | | | x | |
| 212 | x | | | | | | | | x | | | | | | x | | | | | | | | | | x |
| 213 | x | | | | | | | | x | | | | | | | x | x | | | | | | | | |
| 214 | x | | | | | | | | x | | | | | | | x | | x | | | | | | | |
| 215 | x | | | | | | | | x | | | | | | | x | | | x | | | | | | |
| 216 | x | | | | | | | | x | | | | | | | x | | | | x | | | | | |
| 217 | x | | | | | | | | x | | | | | | | x | | | | | x | | | | |
| 218 | x | | | | | | | | x | | | | | | | x | | | | | | x | | | |
| 219 | x | | | | | | | | x | | | | | | | x | | | | | | | x | | |
| 220 | x | | | | | | | | x | | | | | | | x | | | | | | | | x | |
| 221 | x | | | | | | | | x | | | | | | | x | | | | | | | | | x |
| 222 | x | | | | | | | | | x | x | | | | | | x | | | | | | | | |
| 223 | x | | | | | | | | | x | x | | | | | | | x | | | | | | | |
| 224 | x | | | | | | | | | x | x | | | | | | | | x | | | | | | |
| 225 | x | | | | | | | | | x | x | | | | | | | | | x | | | | | |
| 226 | x | | | | | | | | | x | x | | | | | | | | | | x | | | | |
| 227 | x | | | | | | | | | x | x | | | | | | | | | | | x | | | |
| 228 | x | | | | | | | | | x | x | | | | | | | | | | | | x | | |
| 229 | x | | | | | | | | | x | x | | | | | | | | | | | | | x | |
| 230 | x | | | | | | | | | x | x | | | | | | | | | | | | | | x |
| 231 | x | | | | | | | | | x | | x | | | | | x | | | | | | | | |
| 232 | x | | | | | | | | | x | | x | | | | | | x | | | | | | | |
| 233 | x | | | | | | | | | x | | x | | | | | | | x | | | | | | |
| 234 | x | | | | | | | | | x | | x | | | | | | | | x | | | | | |
| 235 | x | | | | | | | | | x | | x | | | | | | | | | x | | | | |
| 236 | x | | | | | | | | | x | | x | | | | | | | | | | x | | | |
| 237 | x | | | | | | | | | x | | x | | | | | | | | | | | x | | |
| 238 | x | | | | | | | | | x | | x | | | | | | | | | | | | x | |
| 239 | x | | | | | | | | | x | | x | | | | | | | | | | | | | x |
| 240 | x | | | | | | | | | x | | | x | | | | x | | | | | | | | |
| 241 | x | | | | | | | | | x | | | x | | | | | x | | | | | | | |
| 242 | x | | | | | | | | | x | | | x | | | | | | x | | | | | | |
| 243 | x | | | | | | | | | x | | | x | | | | | | | x | | | | | |
| 244 | x | | | | | | | | | x | | | x | | | | | | | | x | | | | |
| 245 | x | | | | | | | | | x | | | x | | | | | | | | | x | | | |
| 246 | x | | | | | | | | | x | | | x | | | | | | | | | | | | |
| 247 | x | | | | | | | | | x | | | x | | | | | | | | | | x | | |
| 248 | x | | | | | | | | | x | | | x | | | | | | | | | | | x | |
| 249 | x | | | | | | | | | x | | | x | | | | | | | | | | | | x |
| 250 | x | | | | | | | | | x | | | | x | | | x | | | | | | | | |
| 251 | x | | | | | | | | | x | | | | x | | | | x | | | | | | | |
| 252 | x | | | | | | | | | x | | | | x | | | | | x | | | | | | |
| 253 | x | | | | | | | | | x | | | | x | | | | | | x | | | | | |
| 254 | x | | | | | | | | | x | | | | x | | | | | | | x | | | | |
| 255 | x | | | | | | | | | x | | | | x | | | | | | | | x | | | |
| 256 | x | | | | | | | | | x | | | | x | | | | | | | | | x | | |
| 257 | x | | | | | | | | | x | | | | x | | | | | | | | | | x | |
| 258 | x | | | | | | | | | x | | | | x | | | | | | | | | | | x |
| 259 | x | | | | | | | | | x | | | | | x | | x | | | | | | | | |
| 260 | x | | | | | | | | | x | | | | | x | | | x | | | | | | | |
| 261 | x | | | | | | | | | x | | | | | x | | | | x | | | | | | |
| 262 | x | | | | | | | | | x | | | | | x | | | | | x | | | | | |
| 263 | x | | | | | | | | | x | | | | | x | | | | | | x | | | | |
| 264 | x | | | | | | | | | x | | | | | x | | | | | | | x | | | |
| 265 | x | | | | | | | | | x | | | | | x | | | | | | | | x | | |
| 266 | x | | | | | | | | | x | | | | | x | | | | | | | | | x | |
| 267 | x | | | | | | | | | x | | | | | x | | | | | | | | | | x |
| 268 | x | | | | | | | | | x | | | | | | x | x | | | | | | | | |
| 269 | x | | | | | | | | | x | | | | | | x | | x | | | | | | | |
| 270 | x | | | | | | | | | x | | | | | | x | | | x | | | | | | |
| 271 | x | | | | | | | | | x | | | | | | x | | | | x | | | | | |
| 272 | x | | | | | | | | | x | | | | | | x | | | | | x | | | | |
| 273 | x | | | | | | | | | x | | | | | | x | | | | | | x | | | |
| 274 | x | | | | | | | | | x | | | | | | x | | | | | | | x | | |
| 275 | x | | | | | | | | | x | | | | | | x | | | | | | | | x | |
| 276 | x | | | | | | | | | x | | | | | | x | | | | | | | | | x |
| 277 | | x | | | | x | | | | | x | | | | | | x | | | | | | | | |
| 278 | | x | | | | x | | | | | x | | | | | | | x | | | | | | | |
| 279 | | x | | | | x | | | | | x | | | | | | | | x | | | | | | |
| 280 | | x | | | | x | | | | | x | | | | | | | | | x | | | | | |
| 281 | | x | | | | x | | | | | x | | | | | | | | | | x | | | | |
| 282 | | x | | | | x | | | | | x | | | | | | | | | | | x | | | |
| 283 | | x | | | | x | | | | | x | | | | | | | | | | | | x | | |
| 284 | | x | | | | x | | | | | x | | | | | | | | | | | | | x | |
| 285 | | x | | | | x | | | | | x | | | | | | | | | | | | | | x |
| 286 | | x | | | | x | | | | | | x | | | | | x | | | | | | | | |
| 287 | | x | | | | x | | | | | | x | | | | | | x | | | | | | | |
| 288 | | x | | | | x | | | | | | x | | | | | | | x | | | | | | |
| 289 | | x | | | | x | | | | | | x | | | | | | | | x | | | | | |
| 290 | | x | | | | x | | | | | | x | | | | | | | | | x | | | | |
| 291 | | x | | | | x | | | | | | x | | | | | | | | | | x | | | |
| 292 | | x | | | | x | | | | | | x | | | | | | | | | | | x | | |
| 293 | | x | | | | x | | | | | | x | | | | | | | | | | | | x | |
| 294 | | x | | | | x | | | | | | x | | | | | | | | | | | | | x |
| 295 | | x | | | | x | | | | | | | x | | | | x | | | | | | | | |
| 296 | | x | | | | x | | | | | | | x | | | | | x | | | | | | | |
| 297 | | x | | | | x | | | | | | | x | | | | | | x | | | | | | |
| 298 | | x | | | | x | | | | | | | x | | | | | | | x | | | | | |
| 299 | | x | | | | x | | | | | | | x | | | | | | | | x | | | | |
| 300 | | x | | | | x | | | | | | | x | | | | | | | | | x | | | |
| 301 | | x | | | | x | | | | | | | x | | | | | | | | | | | | |
| 302 | | x | | | | x | | | | | | | x | | | | | | | | | | x | | |
| 303 | | x | | | | x | | | | | | | x | | | | | | | | | | | x | |
| 304 | | x | | | | x | | | | | | | x | | | | | | | | | | | | x |
| 305 | | x | | | | x | | | | | | | | x | | | x | | | | | | | | |
| 306 | | x | | | | x | | | | | | | | x | | | | x | | | | | | | |
| 307 | | x | | | | x | | | | | | | | x | | | | | x | | | | | | |
| 308 | | x | | | | x | | | | | | | | x | | | | | | x | | | | | |
| 309 | | x | | | | x | | | | | | | | x | | | | | | | x | | | | |
| 310 | | x | | | | x | | | | | | | | x | | | | | | | | x | | | |
| 311 | | x | | | | x | | | | | | | | x | | | | | | | | | x | | |
| 312 | | x | | | | x | | | | | | | | x | | | | | | | | | | x | |
| 313 | | x | | | | x | | | | | | | | x | | | | | | | | | | | x |
| 314 | | x | | | | x | | | | | | | | | x | | x | | | | | | | | |
| 315 | | x | | | | x | | | | | | | | | x | | | x | | | | | | | |
| 316 | | x | | | | x | | | | | | | | | x | | | | x | | | | | | |
| 317 | | x | | | | x | | | | | | | | | x | | | | | x | | | | | |
| 318 | | x | | | | x | | | | | | | | | x | | | | | | x | | | | |
| 319 | | x | | | | x | | | | | | | | | x | | | | | | | x | | | |
| 320 | | x | | | | x | | | | | | | | | x | | | | | | | | x | | |
| 321 | | x | | | | x | | | | | | | | | x | | | | | | | | | x | |
| 322 | | x | | | | x | | | | | | | | | x | | | | | | | | | | x |
| 323 | | x | | | | x | | | | | | | | | | x | x | | | | | | | | |
| 324 | | x | | | | x | | | | | | | | | | x | | x | | | | | | | |
| 325 | | x | | | | x | | | | | | | | | | x | | | x | | | | | | |
| 326 | | x | | | | x | | | | | | | | | | x | | | | x | | | | | |
| 327 | | x | | | | x | | | | | | | | | | x | | | | | x | | | | |
| 328 | | x | | | | x | | | | | | | | | | x | | | | | | x | | | |
| 329 | | x | | | | x | | | | | | | | | | x | | | | | | | x | | |
| 330 | | x | | | | x | | | | | | | | | | x | | | | | | | | x | |
| 331 | | x | | | | x | | | | | | | | | | x | | | | | | | | | x |
| 332 | | x | | | | | x | | | | x | | | | | | x | | | | | | | | |
| 333 | | x | | | | | x | | | | x | | | | | | | x | | | | | | | |
| 334 | | x | | | | | x | | | | x | | | | | | | | x | | | | | | |
| 335 | | x | | | | | x | | | | x | | | | | | | | | x | | | | | |
| 336 | | x | | | | | x | | | | x | | | | | | | | | | x | | | | |
| 337 | | x | | | | | x | | | | x | | | | | | | | | | | x | | | |
| 338 | | x | | | | | x | | | | x | | | | | | | | | | | | x | | |
| 339 | | x | | | | | x | | | | x | | | | | | | | | | | | | x | |
| 340 | | x | | | | | x | | | | x | | | | | | | | | | | | | | x |
| 341 | | x | | | | | x | | | | | x | | | | | x | | | | | | | | |
| 342 | | x | | | | | x | | | | | x | | | | | | x | | | | | | | |
| 343 | | x | | | | | x | | | | | x | | | | | | | x | | | | | | |
| 344 | | x | | | | | x | | | | | x | | | | | | | | x | | | | | |
| 345 | | x | | | | | x | | | | | x | | | | | | | | | x | | | | |
| 346 | | x | | | | | x | | | | | x | | | | | | | | | | x | | | |
| 347 | | x | | | | | x | | | | | x | | | | | | | | | | | x | | |
| 348 | | x | | | | | x | | | | | x | | | | | | | | | | | | x | |
| 349 | | x | | | | | x | | | | | x | | | | | | | | | | | | | x |
| 350 | | x | | | | | x | | | | | | x | | | | x | | | | | | | | |
| 351 | | x | | | | | x | | | | | | x | | | | | x | | | | | | | |
| 352 | | x | | | | | x | | | | | | x | | | | | | x | | | | | | |
| 353 | | x | | | | | x | | | | | | x | | | | | | | x | | | | | |
| 354 | | x | | | | | x | | | | | | x | | | | | | | | x | | | | |
| 355 | | x | | | | | x | | | | | | x | | | | | | | | | x | | | |
| 356 | | x | | | | | x | | | | | | x | | | | | | | | | | | | |
| 357 | | x | | | | | x | | | | | | x | | | | | | | | | | x | | |
| 358 | | x | | | | | x | | | | | | x | | | | | | | | | | | x | |
| 359 | | x | | | | | x | | | | | | x | | | | | | | | | | | | x |
| 360 | | x | | | | | x | | | | | | | x | | | x | | | | | | | | |
| 361 | | x | | | | | x | | | | | | | x | | | | x | | | | | | | |
| 362 | | x | | | | | x | | | | | | | x | | | | | x | | | | | | |
| 363 | | x | | | | | x | | | | | | | x | | | | | | x | | | | | |
| 364 | | x | | | | | x | | | | | | | x | | | | | | | x | | | | |
| 365 | | x | | | | | x | | | | | | | x | | | | | | | | x | | | |
| 366 | | x | | | | | x | | | | | | | x | | | | | | | | | x | | |
| 367 | | x | | | | | x | | | | | | | x | | | | | | | | | | x | |
| 368 | | x | | | | | x | | | | | | | x | | | | | | | | | | | x |
| 369 | | x | | | | | x | | | | | | | | x | | x | | | | | | | | |
| 370 | | x | | | | | x | | | | | | | | x | | | x | | | | | | | |
| 371 | | x | | | | | x | | | | | | | | x | | | | x | | | | | | |
| 372 | | x | | | | | x | | | | | | | | x | | | | | x | | | | | |
| 373 | | x | | | | | x | | | | | | | | x | | | | | | x | | | | |
| 374 | | x | | | | | x | | | | | | | | x | | | | | | | x | | | |
| 375 | | x | | | | | x | | | | | | | | x | | | | | | | | x | | |
| 376 | | x | | | | | x | | | | | | | | x | | | | | | | | | x | |
| 377 | | x | | | | | x | | | | | | | | x | | | | | | | | | | x |
| 378 | | x | | | | | x | | | | | | | | | x | x | | | | | | | | |
| 379 | | x | | | | | x | | | | | | | | | x | | x | | | | | | | |
| 380 | | x | | | | | x | | | | | | | | | x | | | x | | | | | | |
| 381 | | x | | | | | x | | | | | | | | | x | | | | x | | | | | |
| 382 | | x | | | | | x | | | | | | | | | x | | | | | x | | | | |
| 383 | | x | | | | | x | | | | | | | | | x | | | | | | x | | | |
| 384 | | x | | | | | x | | | | | | | | | x | | | | | | | x | | |
| 385 | | x | | | | | x | | | | | | | | | x | | | | | | | | x | |
| 386 | | x | | | | | x | | | | | | | | | x | | | | | | | | | x |
| 387 | | x | | | | | | x | | | x | | | | | | x | | | | | | | | |
| 388 | | x | | | | | | x | | | x | | | | | | | x | | | | | | | |
| 389 | | x | | | | | | x | | | x | | | | | | | | x | | | | | | |
| 390 | | x | | | | | | x | | | x | | | | | | | | | x | | | | | |
| 391 | | x | | | | | | x | | | x | | | | | | | | | | x | | | | |
| 392 | | x | | | | | | x | | | x | | | | | | | | | | | x | | | |
| 393 | | x | | | | | | x | | | x | | | | | | | | | | | | x | | |
| 394 | | x | | | | | | x | | | x | | | | | | | | | | | | | x | |
| 395 | | x | | | | | | x | | | x | | | | | | | | | | | | | | x |
| 396 | | x | | | | | | x | | | | x | | | | | x | | | | | | | | |
| 397 | | x | | | | | | x | | | | x | | | | | | x | | | | | | | |
| 398 | | x | | | | | | x | | | | x | | | | | | | x | | | | | | |
| 399 | | x | | | | | | x | | | | x | | | | | | | | x | | | | | |
| 400 | | x | | | | | | x | | | | x | | | | | | | | | x | | | | |
| 401 | | x | | | | | | x | | | | x | | | | | | | | | | x | | | |
| 402 | | x | | | | | | x | | | | x | | | | | | | | | | | x | | |
| 403 | | x | | | | | | x | | | | x | | | | | | | | | | | | x | |
| 404 | | x | | | | | | x | | | | x | | | | | | | | | | | | | x |
| 405 | | x | | | | | | x | | | | | x | | | | x | | | | | | | | |
| 406 | | x | | | | | | x | | | | | x | | | | | x | | | | | | | |
| 407 | | x | | | | | | x | | | | | x | | | | | | x | | | | | | |
| 408 | | x | | | | | | x | | | | | x | | | | | | | x | | | | | |
| 409 | | x | | | | | | x | | | | | x | | | | | | | | x | | | | |
| 410 | | x | | | | | | x | | | | | x | | | | | | | | | x | | | |
| 411 | | x | | | | | | x | | | | | x | | | | | | | | | | | | |
| 412 | | x | | | | | | x | | | | | x | | | | | | | | | | x | | |
| 413 | | x | | | | | | x | | | | | x | | | | | | | | | | | x | |
| 414 | | x | | | | | | x | | | | | x | | | | | | | | | | | | x |
| 415 | | x | | | | | | x | | | | | | x | | | x | | | | | | | | |
| 416 | | x | | | | | | x | | | | | | x | | | | x | | | | | | | |
| 417 | | x | | | | | | x | | | | | | x | | | | | x | | | | | | |
| 418 | | x | | | | | | x | | | | | | x | | | | | | x | | | | | |
| 419 | | x | | | | | | x | | | | | | x | | | | | | | x | | | | |
| 420 | | x | | | | | | x | | | | | | x | | | | | | | | x | | | |
| 421 | | x | | | | | | x | | | | | | x | | | | | | | | | x | | |
| 422 | | x | | | | | | x | | | | | | x | | | | | | | | | | x | |
| 423 | | x | | | | | | x | | | | | | x | | | | | | | | | | | x |
| 424 | | x | | | | | | x | | | | | | | x | | x | | | | | | | | |
| 425 | | x | | | | | | x | | | | | | | x | | | x | | | | | | | |
| 426 | | x | | | | | | x | | | | | | | x | | | | x | | | | | | |
| 427 | | x | | | | | | x | | | | | | | x | | | | | x | | | | | |
| 428 | | x | | | | | | x | | | | | | | x | | | | | | x | | | | |
| 429 | | x | | | | | | x | | | | | | | x | | | | | | | x | | | |
| 430 | | x | | | | | | x | | | | | | | x | | | | | | | | x | | |
| 431 | | x | | | | | | x | | | | | | | x | | | | | | | | | x | |
| 432 | | x | | | | | | x | | | | | | | x | | | | | | | | | | x |
| 433 | | x | | | | | | x | | | | | | | | x | x | | | | | | | | |
| 434 | | x | | | | | | x | | | | | | | | x | | x | | | | | | | |
| 435 | | x | | | | | | x | | | | | | | | x | | | x | | | | | | |
| 436 | | x | | | | | | x | | | | | | | | x | | | | x | | | | | |
| 437 | | x | | | | | | x | | | | | | | | x | | | | | x | | | | |
| 438 | | x | | | | | | x | | | | | | | | x | | | | | | x | | | |
| 439 | | x | | | | | | x | | | | | | | | x | | | | | | | x | | |
| 440 | | x | | | | | | x | | | | | | | | x | | | | | | | | x | |
| 441 | | x | | | | | | x | | | | | | | | x | | | | | | | | | x |
| 442 | | x | | | | | | | x | | x | | | | | | x | | | | | | | | |
| 443 | | x | | | | | | | x | | x | | | | | | | x | | | | | | | |
| 444 | | x | | | | | | | x | | x | | | | | | | | x | | | | | | |
| 445 | | x | | | | | | | x | | x | | | | | | | | | x | | | | | |
| 446 | | x | | | | | | | x | | x | | | | | | | | | | x | | | | |
| 447 | | x | | | | | | | x | | x | | | | | | | | | | | x | | | |
| 448 | | x | | | | | | | x | | x | | | | | | | | | | | | x | | |
| 449 | | x | | | | | | | x | | x | | | | | | | | | | | | | x | |
| 450 | | x | | | | | | | x | | x | | | | | | | | | | | | | | x |
| 451 | | x | | | | | | | x | | | x | | | | | x | | | | | | | | |
| 452 | | x | | | | | | | x | | | x | | | | | | x | | | | | | | |
| 453 | | x | | | | | | | x | | | x | | | | | | | x | | | | | | |
| 454 | | x | | | | | | | x | | | x | | | | | | | | x | | | | | |
| 455 | | x | | | | | | | x | | | x | | | | | | | | | x | | | | |
| 456 | | x | | | | | | | x | | | x | | | | | | | | | | x | | | |
| 457 | | x | | | | | | | x | | | x | | | | | | | | | | | x | | |
| 458 | | x | | | | | | | x | | | x | | | | | | | | | | | | x | |
| 459 | | x | | | | | | | x | | | x | | | | | | | | | | | | | x |
| 460 | | x | | | | | | | x | | | | x | | | | x | | | | | | | | |
| 461 | | x | | | | | | | x | | | | x | | | | | x | | | | | | | |
| 462 | | x | | | | | | | x | | | | x | | | | | | x | | | | | | |
| 463 | | x | | | | | | | x | | | | x | | | | | | | x | | | | | |
| 464 | | x | | | | | | | x | | | | x | | | | | | | | x | | | | |
| 465 | | x | | | | | | | x | | | | x | | | | | | | | | x | | | |
| 466 | | x | | | | | | | x | | | | x | | | | | | | | | | | | |
| 467 | | x | | | | | | | x | | | | x | | | | | | | | | | x | | |
| 468 | | x | | | | | | | x | | | | x | | | | | | | | | | | x | |
| 469 | | x | | | | | | | x | | | | x | | | | | | | | | | | | x |
| 470 | | x | | | | | | | x | | | | | x | | | x | | | | | | | | |
| 471 | | x | | | | | | | x | | | | | x | | | | x | | | | | | | |
| 472 | | x | | | | | | | x | | | | | x | | | | | x | | | | | | |
| 473 | | x | | | | | | | x | | | | | x | | | | | | x | | | | | |
| 474 | | x | | | | | | | x | | | | | x | | | | | | | x | | | | |
| 475 | | x | | | | | | | x | | | | | x | | | | | | | | x | | | |
| 476 | | x | | | | | | | x | | | | | x | | | | | | | | | x | | |
| 477 | | x | | | | | | | x | | | | | x | | | | | | | | | | x | |
| 478 | | x | | | | | | | x | | | | | x | | | | | | | | | | | x |
| 479 | | x | | | | | | | x | | | | | | x | | x | | | | | | | | |
| 480 | | x | | | | | | | x | | | | | | x | | | x | | | | | | | |
| 481 | | x | | | | | | | x | | | | | | x | | | | x | | | | | | |
| 482 | | x | | | | | | | x | | | | | | x | | | | | x | | | | | |
| 483 | | x | | | | | | | x | | | | | | x | | | | | | x | | | | |
| 484 | | x | | | | | | | x | | | | | | x | | | | | | | x | | | |
| 485 | | x | | | | | | | x | | | | | | x | | | | | | | | x | | |
| 486 | | x | | | | | | | x | | | | | | x | | | | | | | | | x | |
| 487 | | x | | | | | | | x | | | | | | x | | | | | | | | | | x |
| 488 | | x | | | | | | | x | | | | | | | x | x | | | | | | | | |
| 489 | | x | | | | | | | x | | | | | | | x | | x | | | | | | | |
| 490 | | x | | | | | | | x | | | | | | | x | | | x | | | | | | |
| 491 | | x | | | | | | | x | | | | | | | x | | | | x | | | | | |
| 492 | | x | | | | | | | x | | | | | | | x | | | | | x | | | | |
| 493 | | x | | | | | | | x | | | | | | | x | | | | | | x | | | |
| 494 | | x | | | | | | | x | | | | | | | x | | | | | | | x | | |
| 495 | | x | | | | | | | x | | | | | | | x | | | | | | | | x | |
| 496 | | x | | | | | | | x | | | | | | | x | | | | | | | | | x |
| 497 | | x | | | | | | | | x | x | | | | | | x | | | | | | | | |
| 498 | | x | | | | | | | | x | x | | | | | | | x | | | | | | | |
| 499 | | x | | | | | | | | x | x | | | | | | | | x | | | | | | |
| 500 | | x | | | | | | | | x | x | | | | | | | | | x | | | | | |
| 501 | | x | | | | | | | | x | x | | | | | | | | | | x | | | | |
| 502 | | x | | | | | | | | x | x | | | | | | | | | | | x | | | |
| 503 | | x | | | | | | | | x | x | | | | | | | | | | | | x | | |
| 504 | | x | | | | | | | | x | x | | | | | | | | | | | | | x | |
| 505 | | x | | | | | | | | x | x | | | | | | | | | | | | | | x |
| 506 | | x | | | | | | | | x | | x | | | | | x | | | | | | | | |
| 507 | | x | | | | | | | | x | | x | | | | | | x | | | | | | | |
| 508 | | x | | | | | | | | x | | x | | | | | | | x | | | | | | |
| 509 | | x | | | | | | | | x | | x | | | | | | | | x | | | | | |
| 510 | | x | | | | | | | | x | | x | | | | | | | | | x | | | | |
| 511 | | x | | | | | | | | x | | x | | | | | | | | | | x | | | |
| 512 | | x | | | | | | | | x | | x | | | | | | | | | | | x | | |
| 513 | | x | | | | | | | | x | | x | | | | | | | | | | | | x | |
| 514 | | x | | | | | | | | x | | x | | | | | | | | | | | | | x |
| 515 | | x | | | | | | | | x | | | x | | | | x | | | | | | | | |
| 516 | | x | | | | | | | | x | | | x | | | | | x | | | | | | | |
| 517 | | x | | | | | | | | x | | | x | | | | | | x | | | | | | |
| 518 | | x | | | | | | | | x | | | x | | | | | | | x | | | | | |
| 519 | | x | | | | | | | | x | | | x | | | | | | | | x | | | | |
| 520 | | x | | | | | | | | x | | | x | | | | | | | | | x | | | |
| 521 | | x | | | | | | | | x | | | x | | | | | | | | | | | | |
| 522 | | x | | | | | | | | x | | | x | | | | | | | | | | x | | |
| 523 | | x | | | | | | | | x | | | x | | | | | | | | | | | x | |
| 524 | | x | | | | | | | | x | | | x | | | | | | | | | | | | x |
| 525 | | x | | | | | | | | x | | | | x | | | x | | | | | | | | |
| 526 | | x | | | | | | | | x | | | | x | | | | x | | | | | | | |
| 527 | | x | | | | | | | | x | | | | x | | | | | x | | | | | | |
| 528 | | x | | | | | | | | x | | | | x | | | | | | x | | | | | |
| 529 | | x | | | | | | | | x | | | | x | | | | | | | x | | | | |
| 530 | | x | | | | | | | | x | | | | x | | | | | | | | x | | | |
| 531 | | x | | | | | | | | x | | | | x | | | | | | | | | x | | |
| 532 | | x | | | | | | | | x | | | | x | | | | | | | | | | x | |
| 533 | | x | | | | | | | | x | | | | x | | | | | | | | | | | x |
| 534 | | x | | | | | | | | x | | | | | x | | x | | | | | | | | |
| 535 | | x | | | | | | | | x | | | | | x | | | x | | | | | | | |
| 536 | | x | | | | | | | | x | | | | | x | | | | x | | | | | | |
| 537 | | x | | | | | | | | x | | | | | x | | | | | x | | | | | |
| 538 | | x | | | | | | | | x | | | | | x | | | | | | x | | | | |
| 539 | | x | | | | | | | | x | | | | | x | | | | | | | x | | | |
| 540 | | x | | | | | | | | x | | | | | x | | | | | | | | x | | |
| 541 | | x | | | | | | | | x | | | | | x | | | | | | | | | x | |
| 542 | | x | | | | | | | | x | | | | | x | | | | | | | | | | x |
| 543 | | x | | | | | | | | x | | | | | | x | x | | | | | | | | |
| 544 | | x | | | | | | | | x | | | | | | x | | x | | | | | | | |
| 545 | | x | | | | | | | | x | | | | | | x | | | x | | | | | | |
| 546 | | x | | | | | | | | x | | | | | | x | | | | x | | | | | |
| 547 | | x | | | | | | | | x | | | | | | x | | | | | x | | | | |
| 548 | | x | | | | | | | | x | | | | | | x | | | | | | x | | | |
| 549 | | x | | | | | | | | x | | | | | | x | | | | | | | x | | |
| 550 | | x | | | | | | | | x | | | | | | x | | | | | | | | x | |
| 551 | | x | | | | | | | | x | | | | | | x | | | | | | | | | x |
| 552 | | | x | | | x | | | | | x | | | | | | x | | | | | | | | |
| 553 | | | x | | | x | | | | | x | | | | | | | x | | | | | | | |
| 554 | | | x | | | x | | | | | x | | | | | | | | x | | | | | | |
| 555 | | | x | | | x | | | | | x | | | | | | | | | x | | | | | |
| 556 | | | x | | | x | | | | | x | | | | | | | | | | x | | | | |
| 557 | | | x | | | x | | | | | x | | | | | | | | | | | x | | | |
| 558 | | | x | | | x | | | | | x | | | | | | | | | | | | x | | |
| 559 | | | x | | | x | | | | | x | | | | | | | | | | | | | x | |
| 560 | | | x | | | x | | | | | x | | | | | | | | | | | | | | x |
| 561 | | | x | | | x | | | | | | x | | | | | x | | | | | | | | |
| 562 | | | x | | | x | | | | | | x | | | | | | x | | | | | | | |
| 563 | | | x | | | x | | | | | | x | | | | | | | x | | | | | | |
| 564 | | | x | | | x | | | | | | x | | | | | | | | x | | | | | |
| 565 | | | x | | | x | | | | | | x | | | | | | | | | x | | | | |
| 566 | | | x | | | x | | | | | | x | | | | | | | | | | x | | | |
| 567 | | | x | | | x | | | | | | x | | | | | | | | | | | x | | |
| 568 | | | x | | | x | | | | | | x | | | | | | | | | | | | x | |
| 569 | | | x | | | x | | | | | | x | | | | | | | | | | | | | x |
| 570 | | | x | | | x | | | | | | | x | | | | x | | | | | | | | |
| 571 | | | x | | | x | | | | | | | x | | | | | x | | | | | | | |
| 572 | | | x | | | x | | | | | | | x | | | | | | x | | | | | | |
| 573 | | | x | | | x | | | | | | | x | | | | | | | x | | | | | |
| 574 | | | x | | | x | | | | | | | x | | | | | | | | x | | | | |
| 575 | | | x | | | x | | | | | | | x | | | | | | | | | x | | | |
| 576 | | | x | | | x | | | | | | | x | | | | | | | | | | | | |
| 577 | | | x | | | x | | | | | | | x | | | | | | | | | | x | | |
| 578 | | | x | | | x | | | | | | | x | | | | | | | | | | | x | |
| 579 | | | x | | | x | | | | | | | x | | | | | | | | | | | | x |
| 580 | | | x | | | x | | | | | | | | x | | | x | | | | | | | | |
| 581 | | | x | | | x | | | | | | | | x | | | | x | | | | | | | |
| 582 | | | x | | | x | | | | | | | | x | | | | | x | | | | | | |
| 583 | | | x | | | x | | | | | | | | x | | | | | | x | | | | | |
| 584 | | | x | | | x | | | | | | | | x | | | | | | | x | | | | |
| 585 | | | x | | | x | | | | | | | | x | | | | | | | | x | | | |
| 586 | | | x | | | x | | | | | | | | x | | | | | | | | | x | | |
| 587 | | | x | | | x | | | | | | | | x | | | | | | | | | | x | |
| 588 | | | x | | | x | | | | | | | | x | | | | | | | | | | | x |
| 589 | | | x | | | x | | | | | | | | | x | | x | | | | | | | | |
| 590 | | | x | | | x | | | | | | | | | x | | | x | | | | | | | |
| 591 | | | x | | | x | | | | | | | | | x | | | | x | | | | | | |
| 592 | | | x | | | x | | | | | | | | | x | | | | | x | | | | | |
| 593 | | | x | | | x | | | | | | | | | x | | | | | | x | | | | |
| 594 | | | x | | | x | | | | | | | | | x | | | | | | | x | | | |
| 595 | | | x | | | x | | | | | | | | | x | | | | | | | | x | | |
| 596 | | | x | | | x | | | | | | | | | x | | | | | | | | | x | |
| 597 | | | x | | | x | | | | | | | | | x | | | | | | | | | | x |
| 598 | | | x | | | x | | | | | | | | | | x | x | | | | | | | | |
| 599 | | | x | | | x | | | | | | | | | | x | | x | | | | | | | |
| 600 | | | x | | | x | | | | | | | | | | x | | | x | | | | | | |
| 601 | | | x | | | x | | | | | | | | | | x | | | | x | | | | | |
| 602 | | | x | | | x | | | | | | | | | | x | | | | | x | | | | |
| 603 | | | x | | | x | | | | | | | | | | x | | | | | | x | | | |
| 604 | | | x | | | x | | | | | | | | | | x | | | | | | | x | | |
| 605 | | | x | | | x | | | | | | | | | | x | | | | | | | | x | |
| 606 | | | x | | | x | | | | | | | | | | x | | | | | | | | | x |
| 607 | | | x | | | | x | | | | x | | | | | | x | | | | | | | | |
| 608 | | | x | | | | x | | | | x | | | | | | | x | | | | | | | |
| 609 | | | x | | | | x | | | | x | | | | | | | | x | | | | | | |
| 610 | | | x | | | | x | | | | x | | | | | | | | | x | | | | | |
| 611 | | | x | | | | x | | | | x | | | | | | | | | | x | | | | |
| 612 | | | x | | | | x | | | | x | | | | | | | | | | | x | | | |
| 613 | | | x | | | | x | | | | x | | | | | | | | | | | | x | | |
| 614 | | | x | | | | x | | | | x | | | | | | | | | | | | | x | |
| 615 | | | x | | | | x | | | | x | | | | | | | | | | | | | | x |
| 616 | | | x | | | | x | | | | | x | | | | | x | | | | | | | | |
| 617 | | | x | | | | x | | | | | x | | | | | | x | | | | | | | |
| 618 | | | x | | | | x | | | | | x | | | | | | | x | | | | | | |
| 619 | | | x | | | | x | | | | | x | | | | | | | | x | | | | | |
| 620 | | | x | | | | x | | | | | x | | | | | | | | | x | | | | |
| 621 | | | x | | | | x | | | | | x | | | | | | | | | | x | | | |
| 622 | | | x | | | | x | | | | | x | | | | | | | | | | | x | | |
| 623 | | | x | | | | x | | | | | x | | | | | | | | | | | | x | |
| 624 | | | x | | | | x | | | | | x | | | | | | | | | | | | | x |
| 625 | | | x | | | | x | | | | | | x | | | | x | | | | | | | | |
| 626 | | | x | | | | x | | | | | | x | | | | | x | | | | | | | |
| 627 | | | x | | | | x | | | | | | x | | | | | | x | | | | | | |
| 628 | | | x | | | | x | | | | | | x | | | | | | | x | | | | | |
| 629 | | | x | | | | x | | | | | | x | | | | | | | | x | | | | |
| 630 | | | x | | | | x | | | | | | x | | | | | | | | | x | | | |
| 631 | | | x | | | | x | | | | | | x | | | | | | | | | | | | |
| 632 | | | x | | | | x | | | | | | x | | | | | | | | | | x | | |
| 633 | | | x | | | | x | | | | | | x | | | | | | | | | | | x | |
| 634 | | | x | | | | x | | | | | | x | | | | | | | | | | | | x |
| 635 | | | x | | | | x | | | | | | | x | | | x | | | | | | | | |
| 636 | | | x | | | | x | | | | | | | x | | | | x | | | | | | | |
| 637 | | | x | | | | x | | | | | | | x | | | | | x | | | | | | |
| 638 | | | x | | | | x | | | | | | | x | | | | | | x | | | | | |
| 639 | | | x | | | | x | | | | | | | x | | | | | | | x | | | | |
| 640 | | | x | | | | x | | | | | | | x | | | | | | | | x | | | |
| 641 | | | x | | | | x | | | | | | | x | | | | | | | | | x | | |
| 642 | | | x | | | | x | | | | | | | x | | | | | | | | | | x | |
| 643 | | | x | | | | x | | | | | | | x | | | | | | | | | | | x |
| 644 | | | x | | | | x | | | | | | | | x | | x | | | | | | | | |
| 645 | | | x | | | | x | | | | | | | | x | | | x | | | | | | | |
| 646 | | | x | | | | x | | | | | | | | x | | | | x | | | | | | |
| 647 | | | x | | | | x | | | | | | | | x | | | | | x | | | | | |
| 648 | | | x | | | | x | | | | | | | | x | | | | | | x | | | | |
| 649 | | | x | | | | x | | | | | | | | x | | | | | | | x | | | |
| 650 | | | x | | | | x | | | | | | | | x | | | | | | | | x | | |
| 651 | | | x | | | | x | | | | | | | | x | | | | | | | | | x | |
| 652 | | | x | | | | x | | | | | | | | x | | | | | | | | | | x |
| 653 | | | x | | | | x | | | | | | | | | x | x | | | | | | | | |
| 654 | | | x | | | | x | | | | | | | | | x | | x | | | | | | | |
| 655 | | | x | | | | x | | | | | | | | | x | | | x | | | | | | |
| 656 | | | x | | | | x | | | | | | | | | x | | | | x | | | | | |
| 657 | | | x | | | | x | | | | | | | | | x | | | | | x | | | | |
| 658 | | | x | | | | x | | | | | | | | | x | | | | | | x | | | |
| 659 | | | x | | | | x | | | | | | | | | x | | | | | | | x | | |
| 660 | | | x | | | | x | | | | | | | | | x | | | | | | | | x | |
| 661 | | | x | | | | x | | | | | | | | | x | | | | | | | | | x |
| 662 | | | x | | | | | x | | | x | | | | | | x | | | | | | | | |
| 663 | | | x | | | | | x | | | x | | | | | | | x | | | | | | | |
| 664 | | | x | | | | | x | | | x | | | | | | | | x | | | | | | |
| 665 | | | x | | | | | x | | | x | | | | | | | | | x | | | | | |
| 666 | | | x | | | | | x | | | x | | | | | | | | | | x | | | | |
| 667 | | | x | | | | | x | | | x | | | | | | | | | | | x | | | |
| 668 | | | x | | | | | x | | | x | | | | | | | | | | | | x | | |
| 669 | | | x | | | | | x | | | x | | | | | | | | | | | | | x | |
| 670 | | | x | | | | | x | | | x | | | | | | | | | | | | | | x |
| 671 | | | x | | | | | x | | | | x | | | | | x | | | | | | | | |
| 672 | | | x | | | | | x | | | | x | | | | | | x | | | | | | | |
| 673 | | | x | | | | | x | | | | x | | | | | | | x | | | | | | |
| 674 | | | x | | | | | x | | | | x | | | | | | | | x | | | | | |
| 675 | | | x | | | | | x | | | | x | | | | | | | | | x | | | | |
| 676 | | | x | | | | | x | | | | x | | | | | | | | | | x | | | |
| 677 | | | x | | | | | x | | | | x | | | | | | | | | | | x | | |
| 678 | | | x | | | | | x | | | | x | | | | | | | | | | | | x | |
| 679 | | | x | | | | | x | | | | x | | | | | | | | | | | | | x |
| 680 | | | x | | | | | x | | | | | x | | | | x | | | | | | | | |
| 681 | | | x | | | | | x | | | | | x | | | | | x | | | | | | | |
| 682 | | | x | | | | | x | | | | | x | | | | | | x | | | | | | |
| 683 | | | x | | | | | x | | | | | x | | | | | | | x | | | | | |
| 684 | | | x | | | | | x | | | | | x | | | | | | | | x | | | | |
| 685 | | | x | | | | | x | | | | | x | | | | | | | | | x | | | |
| 686 | | | x | | | | | x | | | | | x | | | | | | | | | | | | |
| 687 | | | x | | | | | x | | | | | x | | | | | | | | | | x | | |
| 688 | | | x | | | | | x | | | | | x | | | | | | | | | | | x | |
| 689 | | | x | | | | | x | | | | | x | | | | | | | | | | | | x |
| 690 | | | x | | | | | x | | | | | | x | | | x | | | | | | | | |
| 691 | | | x | | | | | x | | | | | | x | | | | x | | | | | | | |
| 692 | | | x | | | | | x | | | | | | x | | | | | x | | | | | | |
| 693 | | | x | | | | | x | | | | | | x | | | | | | x | | | | | |
| 694 | | | x | | | | | x | | | | | | x | | | | | | | x | | | | |
| 695 | | | x | | | | | x | | | | | | x | | | | | | | | x | | | |
| 696 | | | x | | | | | x | | | | | | x | | | | | | | | | x | | |
| 697 | | | x | | | | | x | | | | | | x | | | | | | | | | | x | |
| 698 | | | x | | | | | x | | | | | | x | | | | | | | | | | | x |
| 699 | | | x | | | | | x | | | | | | | x | | x | | | | | | | | |
| 700 | | | x | | | | | x | | | | | | | x | | | x | | | | | | | |
| 701 | | | x | | | | | x | | | | | | | x | | | | x | | | | | | |
| 702 | | | x | | | | | x | | | | | | | x | | | | | x | | | | | |
| 703 | | | x | | | | | x | | | | | | | x | | | | | | x | | | | |
| 704 | | | x | | | | | x | | | | | | | x | | | | | | | x | | | |
| 705 | | | x | | | | | x | | | | | | | x | | | | | | | | x | | |
| 706 | | | x | | | | | x | | | | | | | x | | | | | | | | | x | |
| 707 | | | x | | | | | x | | | | | | | x | | | | | | | | | | x |
| 708 | | | x | | | | | x | | | | | | | | x | x | | | | | | | | |
| 709 | | | x | | | | | x | | | | | | | | x | | x | | | | | | | |
| 710 | | | x | | | | | x | | | | | | | | x | | | x | | | | | | |
| 711 | | | x | | | | | x | | | | | | | | x | | | | x | | | | | |
| 712 | | | x | | | | | x | | | | | | | | x | | | | | x | | | | |
| 713 | | | x | | | | | x | | | | | | | | x | | | | | | x | | | |
| 714 | | | x | | | | | x | | | | | | | | x | | | | | | | x | | |
| 715 | | | x | | | | | x | | | | | | | | x | | | | | | | | x | |
| 716 | | | x | | | | | x | | | | | | | | x | | | | | | | | | x |
| 717 | | | x | | | | | | x | | x | | | | | | x | | | | | | | | |
| 718 | | | x | | | | | | x | | x | | | | | | | x | | | | | | | |
| 719 | | | x | | | | | | x | | x | | | | | | | | x | | | | | | |
| 720 | | | x | | | | | | x | | x | | | | | | | | | x | | | | | |
| 721 | | | x | | | | | | x | | x | | | | | | | | | | x | | | | |
| 722 | | | x | | | | | | x | | x | | | | | | | | | | | x | | | |
| 723 | | | x | | | | | | x | | x | | | | | | | | | | | | x | | |
| 724 | | | x | | | | | | x | | x | | | | | | | | | | | | | x | |
| 725 | | | x | | | | | | x | | x | | | | | | | | | | | | | | x |
| 726 | | | x | | | | | | x | | | x | | | | | x | | | | | | | | |
| 727 | | | x | | | | | | x | | | x | | | | | | x | | | | | | | |
| 728 | | | x | | | | | | x | | | x | | | | | | | x | | | | | | |
| 729 | | | x | | | | | | x | | | x | | | | | | | | x | | | | | |
| 730 | | | x | | | | | | x | | | x | | | | | | | | | x | | | | |
| 731 | | | x | | | | | | x | | | x | | | | | | | | | | x | | | |
| 732 | | | x | | | | | | x | | | x | | | | | | | | | | | x | | |
| 733 | | | x | | | | | | x | | | x | | | | | | | | | | | | x | |
| 734 | | | x | | | | | | x | | | x | | | | | | | | | | | | | x |
| 735 | | | x | | | | | | x | | | | x | | | | x | | | | | | | | |
| 736 | | | x | | | | | | x | | | | x | | | | | x | | | | | | | |
| 737 | | | x | | | | | | x | | | | x | | | | | | x | | | | | | |
| 738 | | | x | | | | | | x | | | | x | | | | | | | x | | | | | |
| 739 | | | x | | | | | | x | | | | x | | | | | | | | x | | | | |
| 740 | | | x | | | | | | x | | | | x | | | | | | | | | x | | | |
| 741 | | | x | | | | | | x | | | | x | | | | | | | | | | | | |
| 742 | | | x | | | | | | x | | | | x | | | | | | | | | | x | | |
| 743 | | | x | | | | | | x | | | | x | | | | | | | | | | | x | |
| 744 | | | x | | | | | | x | | | | x | | | | | | | | | | | | x |
| 745 | | | x | | | | | | x | | | | | x | | | x | | | | | | | | |
| 746 | | | x | | | | | | x | | | | | x | | | | x | | | | | | | |
| 747 | | | x | | | | | | x | | | | | x | | | | | x | | | | | | |
| 748 | | | x | | | | | | x | | | | | x | | | | | | x | | | | | |
| 749 | | | x | | | | | | x | | | | | x | | | | | | | x | | | | |
| 750 | | | x | | | | | | x | | | | | x | | | | | | | | x | | | |
| 751 | | | x | | | | | | x | | | | | x | | | | | | | | | x | | |
| 752 | | | x | | | | | | x | | | | | x | | | | | | | | | | x | |
| 753 | | | x | | | | | | x | | | | | x | | | | | | | | | | | x |
| 754 | | | x | | | | | | x | | | | | | x | | x | | | | | | | | |
| 755 | | | x | | | | | | x | | | | | | x | | | x | | | | | | | |
| 756 | | | x | | | | | | x | | | | | | x | | | | x | | | | | | |
| 757 | | | x | | | | | | x | | | | | | x | | | | | x | | | | | |
| 758 | | | x | | | | | | x | | | | | | x | | | | | | x | | | | |
| 759 | | | x | | | | | | x | | | | | | x | | | | | | | x | | | |
| 760 | | | x | | | | | | x | | | | | | x | | | | | | | | x | | |
| 761 | | | x | | | | | | x | | | | | | x | | | | | | | | | x | |
| 762 | | | x | | | | | | x | | | | | | x | | | | | | | | | | x |
| 763 | | | x | | | | | | x | | | | | | | x | x | | | | | | | | |
| 764 | | | x | | | | | | x | | | | | | | x | | x | | | | | | | |
| 765 | | | x | | | | | | x | | | | | | | x | | | x | | | | | | |
| 766 | | | x | | | | | | x | | | | | | | x | | | | x | | | | | |
| 767 | | | x | | | | | | x | | | | | | | x | | | | | x | | | | |
| 768 | | | x | | | | | | x | | | | | | | x | | | | | | x | | | |
| 769 | | | x | | | | | | x | | | | | | | x | | | | | | | x | | |
| 770 | | | x | | | | | | x | | | | | | | x | | | | | | | | x | |
| 771 | | | x | | | | | | x | | | | | | | x | | | | | | | | | x |
| 772 | | | x | | | | | | | x | x | | | | | | x | | | | | | | | |
| 773 | | | x | | | | | | | x | x | | | | | | | x | | | | | | | |
| 774 | | | x | | | | | | | x | x | | | | | | | | x | | | | | | |
| 775 | | | x | | | | | | | x | x | | | | | | | | | x | | | | | |
| 776 | | | x | | | | | | | x | x | | | | | | | | | | x | | | | |
| 777 | | | x | | | | | | | x | x | | | | | | | | | | | x | | | |
| 778 | | | x | | | | | | | x | x | | | | | | | | | | | | x | | |
| 779 | | | x | | | | | | | x | x | | | | | | | | | | | | | x | |
| 780 | | | x | | | | | | | x | x | | | | | | | | | | | | | | x |
| 781 | | | x | | | | | | | x | | x | | | | | x | | | | | | | | |
| 782 | | | x | | | | | | | x | | x | | | | | | x | | | | | | | |
| 783 | | | x | | | | | | | x | | x | | | | | | | x | | | | | | |
| 784 | | | x | | | | | | | x | | x | | | | | | | | x | | | | | |
| 785 | | | x | | | | | | | x | | x | | | | | | | | | x | | | | |
| 786 | | | x | | | | | | | x | | x | | | | | | | | | | x | | | |
| 787 | | | x | | | | | | | x | | x | | | | | | | | | | | x | | |
| 788 | | | x | | | | | | | x | | x | | | | | | | | | | | | x | |
| 789 | | | x | | | | | | | x | | x | | | | | | | | | | | | | x |
| 790 | | | x | | | | | | | x | | | x | | | | x | | | | | | | | |
| 791 | | | x | | | | | | | x | | | x | | | | | x | | | | | | | |
| 792 | | | x | | | | | | | x | | | x | | | | | | x | | | | | | |
| 793 | | | x | | | | | | | x | | | x | | | | | | | x | | | | | |
| 794 | | | x | | | | | | | x | | | x | | | | | | | | x | | | | |
| 795 | | | x | | | | | | | x | | | x | | | | | | | | | x | | | |
| 796 | | | x | | | | | | | x | | | x | | | | | | | | | | | | |
| 797 | | | x | | | | | | | x | | | x | | | | | | | | | | x | | |
| 798 | | | x | | | | | | | x | | | x | | | | | | | | | | | x | |
| 799 | | | x | | | | | | | x | | | x | | | | | | | | | | | | x |
| 800 | | | x | | | | | | | x | | | | x | | | x | | | | | | | | |
| 801 | | | x | | | | | | | x | | | | x | | | | x | | | | | | | |
| 802 | | | x | | | | | | | x | | | | x | | | | | x | | | | | | |
| 803 | | | x | | | | | | | x | | | | x | | | | | | x | | | | | |
| 804 | | | x | | | | | | | x | | | | x | | | | | | | x | | | | |
| 805 | | | x | | | | | | | x | | | | x | | | | | | | | x | | | |
| 806 | | | x | | | | | | | x | | | | x | | | | | | | | | x | | |
| 807 | | | x | | | | | | | x | | | | x | | | | | | | | | | x | |
| 808 | | | x | | | | | | | x | | | | x | | | | | | | | | | | x |
| 809 | | | x | | | | | | | x | | | | | x | | x | | | | | | | | |
| 810 | | | x | | | | | | | x | | | | | x | | | x | | | | | | | |
| 811 | | | x | | | | | | | x | | | | | x | | | | x | | | | | | |
| 812 | | | x | | | | | | | x | | | | | x | | | | | x | | | | | |
| 813 | | | x | | | | | | | x | | | | | x | | | | | | x | | | | |
| 814 | | | x | | | | | | | x | | | | | x | | | | | | | x | | | |
| 815 | | | x | | | | | | | x | | | | | x | | | | | | | | x | | |
| 816 | | | x | | | | | | | x | | | | | x | | | | | | | | | x | |
| 817 | | | x | | | | | | | x | | | | | x | | | | | | | | | | x |
| 818 | | | x | | | | | | | x | | | | | | x | x | | | | | | | | |
| 819 | | | x | | | | | | | x | | | | | | x | | x | | | | | | | |
| 820 | | | x | | | | | | | x | | | | | | x | | | x | | | | | | |
| 821 | | | x | | | | | | | x | | | | | | x | | | | x | | | | | |
| 822 | | | x | | | | | | | x | | | | | | x | | | | | x | | | | |
| 823 | | | x | | | | | | | x | | | | | | x | | | | | | x | | | |
| 824 | | | x | | | | | | | x | | | | | | x | | | | | | | x | | |
| 825 | | | x | | | | | | | x | | | | | | x | | | | | | | | x | |
| 826 | | | x | | | | | | | x | | | | | | x | | | | | | | | | x |
| 827 | | | | x | | x | | | | | x | | | | | | x | | | | | | | | |
| 828 | | | | x | | x | | | | | x | | | | | | | x | | | | | | | |
| 829 | | | | x | | x | | | | | x | | | | | | | | x | | | | | | |
| 830 | | | | x | | x | | | | | x | | | | | | | | | x | | | | | |
| 831 | | | | x | | x | | | | | x | | | | | | | | | | x | | | | |
| 832 | | | | x | | x | | | | | x | | | | | | | | | | | x | | | |
| 833 | | | | x | | x | | | | | x | | | | | | | | | | | | x | | |
| 834 | | | | x | | x | | | | | x | | | | | | | | | | | | | x | |
| 835 | | | | x | | x | | | | | x | | | | | | | | | | | | | | x |
| 836 | | | | x | | x | | | | | | x | | | | | x | | | | | | | | |
| 837 | | | | x | | x | | | | | | x | | | | | | x | | | | | | | |
| 838 | | | | x | | x | | | | | | x | | | | | | | x | | | | | | |
| 839 | | | | x | | x | | | | | | x | | | | | | | | x | | | | | |
| 840 | | | | x | | x | | | | | | x | | | | | | | | | x | | | | |
| 841 | | | | x | | x | | | | | | x | | | | | | | | | | x | | | |
| 842 | | | | x | | x | | | | | | x | | | | | | | | | | | x | | |
| 843 | | | | x | | x | | | | | | x | | | | | | | | | | | | x | |
| 844 | | | | x | | x | | | | | | x | | | | | | | | | | | | | x |
| 845 | | | | x | | x | | | | | | | x | | | | x | | | | | | | | |
| 846 | | | | x | | x | | | | | | | x | | | | | x | | | | | | | |
| 847 | | | | x | | x | | | | | | | x | | | | | | x | | | | | | |
| 848 | | | | x | | x | | | | | | | x | | | | | | | x | | | | | |
| 849 | | | | x | | x | | | | | | | x | | | | | | | | x | | | | |
| 850 | | | | x | | x | | | | | | | x | | | | | | | | | x | | | |
| 851 | | | | x | | x | | | | | | | x | | | | | | | | | | | | |
| 852 | | | | x | | x | | | | | | | x | | | | | | | | | | x | | |
| 853 | | | | x | | x | | | | | | | x | | | | | | | | | | | x | |
| 854 | | | | x | | x | | | | | | | x | | | | | | | | | | | | x |
| 855 | | | | x | | x | | | | | | | | x | | | x | | | | | | | | |
| 856 | | | | x | | x | | | | | | | | x | | | | x | | | | | | | |
| 857 | | | | x | | x | | | | | | | | x | | | | | x | | | | | | |
| 858 | | | | x | | x | | | | | | | | x | | | | | | x | | | | | |
| 859 | | | | x | | x | | | | | | | | x | | | | | | | x | | | | |
| 860 | | | | x | | x | | | | | | | | x | | | | | | | | x | | | |
| 861 | | | | x | | x | | | | | | | | x | | | | | | | | | x | | |
| 862 | | | | x | | x | | | | | | | | x | | | | | | | | | | x | |
| 863 | | | | x | | x | | | | | | | | x | | | | | | | | | | | x |
| 864 | | | | x | | x | | | | | | | | | x | | x | | | | | | | | |
| 865 | | | | x | | x | | | | | | | | | x | | | x | | | | | | | |
| 866 | | | | x | | x | | | | | | | | | x | | | | x | | | | | | |
| 867 | | | | x | | x | | | | | | | | | x | | | | | x | | | | | |
| 868 | | | | x | | x | | | | | | | | | x | | | | | | x | | | | |
| 869 | | | | x | | x | | | | | | | | | x | | | | | | | x | | | |
| 870 | | | | x | | x | | | | | | | | | x | | | | | | | | x | | |
| 871 | | | | x | | x | | | | | | | | | x | | | | | | | | | x | |
| 872 | | | | x | | x | | | | | | | | | x | | | | | | | | | | x |
| 873 | | | | x | | x | | | | | | | | | | x | x | | | | | | | | |
| 874 | | | | x | | x | | | | | | | | | | x | | x | | | | | | | |
| 875 | | | | x | | x | | | | | | | | | | x | | | x | | | | | | |
| 876 | | | | x | | x | | | | | | | | | | x | | | | x | | | | | |
| 877 | | | | x | | x | | | | | | | | | | x | | | | | x | | | | |
| 878 | | | | x | | x | | | | | | | | | | x | | | | | | x | | | |
| 879 | | | | x | | x | | | | | | | | | | x | | | | | | | x | | |
| 880 | | | | x | | x | | | | | | | | | | x | | | | | | | | x | |
| 881 | | | | x | | x | | | | | | | | | | x | | | | | | | | | x |
| 882 | | | | x | | | x | | | | x | | | | | | x | | | | | | | | |
| 883 | | | | x | | | x | | | | x | | | | | | | x | | | | | | | |
| 884 | | | | x | | | x | | | | x | | | | | | | | x | | | | | | |
| 885 | | | | x | | | x | | | | x | | | | | | | | | x | | | | | |
| 886 | | | | x | | | x | | | | x | | | | | | | | | | x | | | | |
| 887 | | | | x | | | x | | | | x | | | | | | | | | | | x | | | |
| 888 | | | | x | | | x | | | | x | | | | | | | | | | | | x | | |
| 889 | | | | x | | | x | | | | x | | | | | | | | | | | | | x | |
| 890 | | | | x | | | x | | | | x | | | | | | | | | | | | | | x |
| 891 | | | | x | | | x | | | | | x | | | | | x | | | | | | | | |
| 892 | | | | x | | | x | | | | | x | | | | | | x | | | | | | | |
| 893 | | | | x | | | x | | | | | x | | | | | | | x | | | | | | |
| 894 | | | | x | | | x | | | | | x | | | | | | | | x | | | | | |
| 895 | | | | x | | | x | | | | | x | | | | | | | | | x | | | | |
| 896 | | | | x | | | x | | | | | x | | | | | | | | | | x | | | |
| 897 | | | | x | | | x | | | | | x | | | | | | | | | | | x | | |
| 898 | | | | x | | | x | | | | | x | | | | | | | | | | | | x | |
| 899 | | | | x | | | x | | | | | x | | | | | | | | | | | | | x |
| 900 | | | | x | | | x | | | | | | x | | | | x | | | | | | | | |
| 901 | | | | x | | | x | | | | | | x | | | | | x | | | | | | | |
| 902 | | | | x | | | x | | | | | | x | | | | | | x | | | | | | |
| 903 | | | | x | | | x | | | | | | x | | | | | | | x | | | | | |
| 904 | | | | x | | | x | | | | | | x | | | | | | | | x | | | | |
| 905 | | | | x | | | x | | | | | | x | | | | | | | | | x | | | |
| 906 | | | | x | | | x | | | | | | x | | | | | | | | | | | | |
| 907 | | | | x | | | x | | | | | | x | | | | | | | | | | x | | |
| 908 | | | | x | | | x | | | | | | x | | | | | | | | | | | x | |
| 909 | | | | x | | | x | | | | | | x | | | | | | | | | | | | x |
| 910 | | | | x | | | x | | | | | | | x | | | x | | | | | | | | |
| 911 | | | | x | | | x | | | | | | | x | | | | x | | | | | | | |
| 912 | | | | x | | | x | | | | | | | x | | | | | x | | | | | | |
| 913 | | | | x | | | x | | | | | | | x | | | | | | x | | | | | |
| 914 | | | | x | | | x | | | | | | | x | | | | | | | x | | | | |
| 915 | | | | x | | | x | | | | | | | x | | | | | | | | x | | | |
| 916 | | | | x | | | x | | | | | | | x | | | | | | | | | x | | |
| 917 | | | | x | | | x | | | | | | | x | | | | | | | | | | x | |
| 918 | | | | x | | | x | | | | | | | x | | | | | | | | | | | x |
| 919 | | | | x | | | x | | | | | | | | x | | x | | | | | | | | |
| 920 | | | | x | | | x | | | | | | | | x | | | x | | | | | | | |
| 921 | | | | x | | | x | | | | | | | | x | | | | x | | | | | | |
| 922 | | | | x | | | x | | | | | | | | x | | | | | x | | | | | |
| 923 | | | | x | | | x | | | | | | | | x | | | | | | x | | | | |
| 924 | | | | x | | | x | | | | | | | | x | | | | | | | x | | | |
| 925 | | | | x | | | x | | | | | | | | x | | | | | | | | x | | |
| 926 | | | | x | | | x | | | | | | | | x | | | | | | | | | x | |
| 927 | | | | x | | | x | | | | | | | | x | | | | | | | | | | x |
| 928 | | | | x | | | x | | | | | | | | | x | x | | | | | | | | |
| 929 | | | | x | | | x | | | | | | | | | x | | x | | | | | | | |
| 930 | | | | x | | | x | | | | | | | | | x | | | x | | | | | | |
| 931 | | | | x | | | x | | | | | | | | | x | | | | x | | | | | |
| 932 | | | | x | | | x | | | | | | | | | x | | | | | x | | | | |
| 933 | | | | x | | | x | | | | | | | | | x | | | | | | x | | | |
| 934 | | | | x | | | x | | | | | | | | | x | | | | | | | x | | |
| 935 | | | | x | | | x | | | | | | | | | x | | | | | | | | x | |
| 936 | | | | x | | | x | | | | | | | | | x | | | | | | | | | x |
| 937 | | | | x | | | | x | | | x | | | | | | x | | | | | | | | |
| 938 | | | | x | | | | x | | | x | | | | | | | x | | | | | | | |
| 939 | | | | x | | | | x | | | x | | | | | | | | x | | | | | | |
| 940 | | | | x | | | | x | | | x | | | | | | | | | x | | | | | |
| 941 | | | | x | | | | x | | | x | | | | | | | | | | x | | | | |
| 942 | | | | x | | | | x | | | x | | | | | | | | | | | x | | | |
| 943 | | | | x | | | | x | | | x | | | | | | | | | | | | x | | |
| 944 | | | | x | | | | x | | | x | | | | | | | | | | | | | x | |
| 945 | | | | x | | | | x | | | x | | | | | | | | | | | | | | x |
| 946 | | | | x | | | | x | | | | x | | | | | x | | | | | | | | |
| 947 | | | | x | | | | x | | | | x | | | | | | x | | | | | | | |
| 948 | | | | x | | | | x | | | | x | | | | | | | x | | | | | | |
| 949 | | | | x | | | | x | | | | x | | | | | | | | x | | | | | |
| 950 | | | | x | | | | x | | | | x | | | | | | | | | x | | | | |
| 951 | | | | x | | | | x | | | | x | | | | | | | | | | x | | | |
| 952 | | | | x | | | | x | | | | x | | | | | | | | | | | x | | |
| 953 | | | | x | | | | x | | | | x | | | | | | | | | | | | x | |
| 954 | | | | x | | | | x | | | | x | | | | | | | | | | | | | x |
| 955 | | | | x | | | | x | | | | | x | | | | x | | | | | | | | |
| 956 | | | | x | | | | x | | | | | x | | | | | x | | | | | | | |
| 957 | | | | x | | | | x | | | | | x | | | | | | x | | | | | | |
| 958 | | | | x | | | | x | | | | | x | | | | | | | x | | | | | |
| 959 | | | | x | | | | x | | | | | x | | | | | | | | x | | | | |
| 960 | | | | x | | | | x | | | | | x | | | | | | | | | x | | | |
| 961 | | | | x | | | | x | | | | | x | | | | | | | | | | | | |
| 962 | | | | x | | | | x | | | | | x | | | | | | | | | | x | | |
| 963 | | | | x | | | | x | | | | | x | | | | | | | | | | | x | |
| 964 | | | | x | | | | x | | | | | x | | | | | | | | | | | | x |
| 965 | | | | x | | | | x | | | | | | x | | | x | | | | | | | | |
| 966 | | | | x | | | | x | | | | | | x | | | | x | | | | | | | |
| 967 | | | | x | | | | x | | | | | | x | | | | | x | | | | | | |
| 968 | | | | x | | | | x | | | | | | x | | | | | | x | | | | | |
| 969 | | | | x | | | | x | | | | | | x | | | | | | | x | | | | |
| 970 | | | | x | | | | x | | | | | | x | | | | | | | | x | | | |
| 971 | | | | x | | | | x | | | | | | x | | | | | | | | | x | | |
| 972 | | | | x | | | | x | | | | | | x | | | | | | | | | | x | |
| 973 | | | | x | | | | x | | | | | | x | | | | | | | | | | | x |
| 974 | | | | x | | | | x | | | | | | | x | | x | | | | | | | | |
| 975 | | | | x | | | | x | | | | | | | x | | | x | | | | | | | |
| 976 | | | | x | | | | x | | | | | | | x | | | | x | | | | | | |
| 977 | | | | x | | | | x | | | | | | | x | | | | | x | | | | | |
| 978 | | | | x | | | | x | | | | | | | x | | | | | | x | | | | |
| 979 | | | | x | | | | x | | | | | | | x | | | | | | | x | | | |
| 980 | | | | x | | | | x | | | | | | | x | | | | | | | | x | | |
| 981 | | | | x | | | | x | | | | | | | x | | | | | | | | | x | |
| 982 | | | | x | | | | x | | | | | | | x | | | | | | | | | | x |
| 983 | | | | x | | | | x | | | | | | | | x | x | | | | | | | | |
| 984 | | | | x | | | | x | | | | | | | | x | | x | | | | | | | |
| 985 | | | | x | | | | x | | | | | | | | x | | | x | | | | | | |
| 986 | | | | x | | | | x | | | | | | | | x | | | | x | | | | | |
| 987 | | | | x | | | | x | | | | | | | | x | | | | | x | | | | |
| 988 | | | | x | | | | x | | | | | | | | x | | | | | | x | | | |
| 989 | | | | x | | | | x | | | | | | | | x | | | | | | | x | | |
| 990 | | | | x | | | | x | | | | | | | | x | | | | | | | | x | |
| 991 | | | | x | | | | x | | | | | | | | x | | | | | | | | | x |
| 992 | | | | x | | | | | x | | x | | | | | | x | | | | | | | | |
| 993 | | | | x | | | | | x | | x | | | | | | | x | | | | | | | |
| 994 | | | | x | | | | | x | | x | | | | | | | | x | | | | | | |
| 995 | | | | x | | | | | x | | x | | | | | | | | | x | | | | | |
| 996 | | | | x | | | | | x | | x | | | | | | | | | | x | | | | |
| 997 | | | | x | | | | | x | | x | | | | | | | | | | | x | | | |
| 998 | | | | x | | | | | x | | x | | | | | | | | | | | | x | | |
| 999 | | | | x | | | | | x | | x | | | | | | | | | | | | | x | |
| 1000 | | | | x | | | | | x | | x | | | | | | | | | | | | | | x |
| 1001 | | | | x | | | | | x | | | x | | | | | x | | | | | | | | |
| 1002 | | | | x | | | | | x | | | x | | | | | | x | | | | | | | |
| 1003 | | | | x | | | | | x | | | x | | | | | | | x | | | | | | |
| 1004 | | | | x | | | | | x | | | x | | | | | | | | x | | | | | |
| 1005 | | | | x | | | | | x | | | x | | | | | | | | | x | | | | |
| 1006 | | | | x | | | | | x | | | x | | | | | | | | | | x | | | |
| 1007 | | | | x | | | | | x | | | x | | | | | | | | | | | x | | |
| 1008 | | | | x | | | | | x | | | x | | | | | | | | | | | | x | |
| 1009 | | | | x | | | | | x | | | x | | | | | | | | | | | | | x |
| 1010 | | | | x | | | | | x | | | | x | | | | x | | | | | | | | |
| 1011 | | | | x | | | | | x | | | | x | | | | | x | | | | | | | |
| 1012 | | | | x | | | | | x | | | | x | | | | | | x | | | | | | |
| 1013 | | | | x | | | | | x | | | | x | | | | | | | x | | | | | |
| 1014 | | | | x | | | | | x | | | | x | | | | | | | | x | | | | |
| 1015 | | | | x | | | | | x | | | | x | | | | | | | | | x | | | |
| 1016 | | | | x | | | | | x | | | | x | | | | | | | | | | | | |
| 1017 | | | | x | | | | | x | | | | x | | | | | | | | | | x | | |
| 1018 | | | | x | | | | | x | | | | x | | | | | | | | | | | x | |
| 1019 | | | | x | | | | | x | | | | x | | | | | | | | | | | | x |
| 1020 | | | | x | | | | | x | | | | | x | | | x | | | | | | | | |
| 1021 | | | | x | | | | | x | | | | | x | | | | x | | | | | | | |
| 1022 | | | | x | | | | | x | | | | | x | | | | | x | | | | | | |
| 1023 | | | | x | | | | | x | | | | | x | | | | | | x | | | | | |
| 1024 | | | | x | | | | | x | | | | | x | | | | | | | x | | | | |
| 1025 | | | | x | | | | | x | | | | | x | | | | | | | | x | | | |
| 1026 | | | | x | | | | | x | | | | | x | | | | | | | | | x | | |
| 1027 | | | | x | | | | | x | | | | | x | | | | | | | | | | x | |
| 1028 | | | | x | | | | | x | | | | | x | | | | | | | | | | | x |
| 1029 | | | | x | | | | | x | | | | | | x | | x | | | | | | | | |
| 1030 | | | | x | | | | | x | | | | | | x | | | x | | | | | | | |
| 1031 | | | | x | | | | | x | | | | | | x | | | | x | | | | | | |
| 1032 | | | | x | | | | | x | | | | | | x | | | | | x | | | | | |
| 1033 | | | | x | | | | | x | | | | | | x | | | | | | x | | | | |
| 1034 | | | | x | | | | | x | | | | | | x | | | | | | | x | | | |
| 1035 | | | | x | | | | | x | | | | | | x | | | | | | | | x | | |
| 1036 | | | | x | | | | | x | | | | | | x | | | | | | | | | x | |
| 1037 | | | | x | | | | | x | | | | | | x | | | | | | | | | | x |
| 1038 | | | | x | | | | | x | | | | | | | x | x | | | | | | | | |
| 1039 | | | | x | | | | | x | | | | | | | x | | x | | | | | | | |
| 1040 | | | | x | | | | | x | | | | | | | x | | | x | | | | | | |
| 1041 | | | | x | | | | | x | | | | | | | x | | | | x | | | | | |
| 1042 | | | | x | | | | | x | | | | | | | x | | | | | x | | | | |
| 1043 | | | | x | | | | | x | | | | | | | x | | | | | | x | | | |
| 1044 | | | | x | | | | | x | | | | | | | x | | | | | | | x | | |
| 1045 | | | | x | | | | | x | | | | | | | x | | | | | | | | x | |
| 1046 | | | | x | | | | | x | | | | | | | x | | | | | | | | | x |
| 1047 | | | | x | | | | | | x | x | | | | | | x | | | | | | | | |
| 1048 | | | | x | | | | | | x | x | | | | | | | x | | | | | | | |
| 1049 | | | | x | | | | | | x | x | | | | | | | | x | | | | | | |
| 1050 | | | | x | | | | | | x | x | | | | | | | | | x | | | | | |
| 1051 | | | | x | | | | | | x | x | | | | | | | | | | x | | | | |
| 1052 | | | | x | | | | | | x | x | | | | | | | | | | | x | | | |
| 1053 | | | | x | | | | | | x | x | | | | | | | | | | | | x | | |
| 1054 | | | | x | | | | | | x | x | | | | | | | | | | | | | x | |
| 1055 | | | | x | | | | | | x | x | | | | | | | | | | | | | | x |
| 1056 | | | | x | | | | | | x | | x | | | | | x | | | | | | | | |
| 1057 | | | | x | | | | | | x | | x | | | | | | x | | | | | | | |
| 1058 | | | | x | | | | | | x | | x | | | | | | | x | | | | | | |
| 1059 | | | | x | | | | | | x | | x | | | | | | | | x | | | | | |
| 1060 | | | | x | | | | | | x | | x | | | | | | | | | x | | | | |
| 1061 | | | | x | | | | | | x | | x | | | | | | | | | | x | | | |
| 1062 | | | | x | | | | | | x | | x | | | | | | | | | | | x | | |
| 1063 | | | | x | | | | | | x | | x | | | | | | | | | | | | x | |
| 1064 | | | | x | | | | | | x | | x | | | | | | | | | | | | | x |
| 1065 | | | | x | | | | | | x | | | x | | | | x | | | | | | | | |
| 1066 | | | | x | | | | | | x | | | x | | | | | x | | | | | | | |
| 1067 | | | | x | | | | | | x | | | x | | | | | | x | | | | | | |
| 1068 | | | | x | | | | | | x | | | x | | | | | | | x | | | | | |
| 1069 | | | | x | | | | | | x | | | x | | | | | | | | x | | | | |
| 1070 | | | | x | | | | | | x | | | x | | | | | | | | | x | | | |
| 1071 | | | | x | | | | | | x | | | x | | | | | | | | | | | | |
| 1072 | | | | x | | | | | | x | | | x | | | | | | | | | | x | | |
| 1073 | | | | x | | | | | | x | | | x | | | | | | | | | | | x | |
| 1074 | | | | x | | | | | | x | | | x | | | | | | | | | | | | x |
| 1075 | | | | x | | | | | | x | | | | x | | | x | | | | | | | | |
| 1076 | | | | x | | | | | | x | | | | x | | | | x | | | | | | | |
| 1077 | | | | x | | | | | | x | | | | x | | | | | x | | | | | | |
| 1078 | | | | x | | | | | | x | | | | x | | | | | | x | | | | | |
| 1079 | | | | x | | | | | | x | | | | x | | | | | | | x | | | | |
| 1080 | | | | x | | | | | | x | | | | x | | | | | | | | x | | | |
| 1081 | | | | x | | | | | | x | | | | x | | | | | | | | | x | | |
| 1082 | | | | x | | | | | | x | | | | x | | | | | | | | | | x | |
| 1083 | | | | x | | | | | | x | | | | x | | | | | | | | | | | x |
| 1084 | | | | x | | | | | | x | | | | | x | | x | | | | | | | | |
| 1085 | | | | x | | | | | | x | | | | | x | | | x | | | | | | | |
| 1086 | | | | x | | | | | | x | | | | | x | | | | x | | | | | | |
| 1087 | | | | x | | | | | | x | | | | | x | | | | | x | | | | | |
| 1088 | | | | x | | | | | | x | | | | | x | | | | | | x | | | | |
| 1089 | | | | x | | | | | | x | | | | | x | | | | | | | x | | | |
| 1090 | | | | x | | | | | | x | | | | | x | | | | | | | | x | | |
| 1091 | | | | x | | | | | | x | | | | | x | | | | | | | | | x | |
| 1092 | | | | x | | | | | | x | | | | | x | | | | | | | | | | x |
| 1093 | | | | x | | | | | | x | | | | | | x | x | | | | | | | | |
| 1094 | | | | x | | | | | | x | | | | | | x | | x | | | | | | | |
| 1095 | | | | x | | | | | | x | | | | | | x | | | x | | | | | | |
| 1096 | | | | x | | | | | | x | | | | | | x | | | | x | | | | | |
| 1097 | | | | x | | | | | | x | | | | | | x | | | | | x | | | | |
| 1098 | | | | x | | | | | | x | | | | | | x | | | | | | x | | | |
| 1099 | | | | x | | | | | | x | | | | | | x | | | | | | | x | | |
| 1100 | | | | x | | | | | | x | | | | | | x | | | | | | | | x | |
| 1101 | | | | x | | | | | | x | | | | | | x | | | | | | | | | x |
| 1102 | | | | | x | x | | | | | x | | | | | | x | | | | | | | | |
| 1103 | | | | | x | x | | | | | x | | | | | | | x | | | | | | | |
| 1104 | | | | | x | x | | | | | x | | | | | | | | x | | | | | | |
| 1105 | | | | | x | x | | | | | x | | | | | | | | | x | | | | | |
| 1106 | | | | | x | x | | | | | x | | | | | | | | | | x | | | | |
| 1107 | | | | | x | x | | | | | x | | | | | | | | | | | x | | | |
| 1108 | | | | | x | x | | | | | x | | | | | | | | | | | | x | | |
| 1109 | | | | | x | x | | | | | x | | | | | | | | | | | | | x | |
| 1110 | | | | | x | x | | | | | x | | | | | | | | | | | | | | x |
| 1111 | | | | | x | x | | | | | | x | | | | | x | | | | | | | | |
| 1112 | | | | | x | x | | | | | | x | | | | | | x | | | | | | | |
| 1113 | | | | | x | x | | | | | | x | | | | | | | x | | | | | | |
| 1114 | | | | | x | x | | | | | | x | | | | | | | | x | | | | | |
| 1115 | | | | | x | x | | | | | | x | | | | | | | | | x | | | | |
| 1116 | | | | | x | x | | | | | | x | | | | | | | | | | x | | | |
| 1117 | | | | | x | x | | | | | | x | | | | | | | | | | | x | | |
| 1118 | | | | | x | x | | | | | | x | | | | | | | | | | | | x | |
| 1119 | | | | | x | x | | | | | | x | | | | | | | | | | | | | x |
| 1120 | | | | | x | x | | | | | | | x | | | | x | | | | | | | | |
| 1121 | | | | | x | x | | | | | | | x | | | | | x | | | | | | | |
| 1122 | | | | | x | x | | | | | | | x | | | | | | x | | | | | | |
| 1123 | | | | | x | x | | | | | | | x | | | | | | | x | | | | | |
| 1124 | | | | | x | x | | | | | | | x | | | | | | | | x | | | | |
| 1125 | | | | | x | x | | | | | | | x | | | | | | | | | x | | | |
| 1126 | | | | | x | x | | | | | | | x | | | | | | | | | | | | |
| 1127 | | | | | x | x | | | | | | | x | | | | | | | | | | x | | |
| 1128 | | | | | x | x | | | | | | | x | | | | | | | | | | | x | |
| 1129 | | | | | x | x | | | | | | | x | | | | | | | | | | | | x |
| 1130 | | | | | x | x | | | | | | | | x | | | x | | | | | | | | |
| 1131 | | | | | x | x | | | | | | | | x | | | | x | | | | | | | |
| 1132 | | | | | x | x | | | | | | | | x | | | | | x | | | | | | |
| 1133 | | | | | x | x | | | | | | | | x | | | | | | x | | | | | |
| 1134 | | | | | x | x | | | | | | | | x | | | | | | | x | | | | |
| 1135 | | | | | x | x | | | | | | | | x | | | | | | | | x | | | |
| 1136 | | | | | x | x | | | | | | | | x | | | | | | | | | x | | |
| 1137 | | | | | x | x | | | | | | | | x | | | | | | | | | | x | |
| 1138 | | | | | x | x | | | | | | | | x | | | | | | | | | | | x |
| 1139 | | | | | x | x | | | | | | | | | x | | x | | | | | | | | |
| 1140 | | | | | x | x | | | | | | | | | x | | | x | | | | | | | |
| 1141 | | | | | x | x | | | | | | | | | x | | | | x | | | | | | |
| 1142 | | | | | x | x | | | | | | | | | x | | | | | x | | | | | |
| 1143 | | | | | x | x | | | | | | | | | x | | | | | | x | | | | |
| 1144 | | | | | x | x | | | | | | | | | x | | | | | | | x | | | |
| 1145 | | | | | x | x | | | | | | | | | x | | | | | | | | x | | |
| 1146 | | | | | x | x | | | | | | | | | x | | | | | | | | | x | |
| 1147 | | | | | x | x | | | | | | | | | x | | | | | | | | | | x |
| 1148 | | | | | x | x | | | | | | | | | | x | x | | | | | | | | |
| 1149 | | | | | x | x | | | | | | | | | | x | | x | | | | | | | |
| 1150 | | | | | x | x | | | | | | | | | | x | | | x | | | | | | |
| 1151 | | | | | x | x | | | | | | | | | | x | | | | x | | | | | |
| 1152 | | | | | x | x | | | | | | | | | | x | | | | | x | | | | |
| 1153 | | | | | x | x | | | | | | | | | | x | | | | | | x | | | |
| 1154 | | | | | x | x | | | | | | | | | | x | | | | | | | x | | |
| 1155 | | | | | x | x | | | | | | | | | | x | | | | | | | | x | |
| 1156 | | | | | x | x | | | | | | | | | | x | | | | | | | | | x |
| 1157 | | | | | x | | x | | | | x | | | | | | x | | | | | | | | |
| 1158 | | | | | x | | x | | | | x | | | | | | | x | | | | | | | |
| 1159 | | | | | x | | x | | | | x | | | | | | | | x | | | | | | |
| 1160 | | | | | x | | x | | | | x | | | | | | | | | x | | | | | |
| 1161 | | | | | x | | x | | | | x | | | | | | | | | | x | | | | |
| 1162 | | | | | x | | x | | | | x | | | | | | | | | | | x | | | |
| 1163 | | | | | x | | x | | | | x | | | | | | | | | | | | x | | |
| 1164 | | | | | x | | x | | | | x | | | | | | | | | | | | | x | |
| 1165 | | | | | x | | x | | | | x | | | | | | | | | | | | | | x |
| 1166 | | | | | x | | x | | | | | x | | | | | x | | | | | | | | |
| 1167 | | | | | x | | x | | | | | x | | | | | | x | | | | | | | |
| 1168 | | | | | x | | x | | | | | x | | | | | | | x | | | | | | |
| 1169 | | | | | x | | x | | | | | x | | | | | | | | x | | | | | |
| 1170 | | | | | x | | x | | | | | x | | | | | | | | | x | | | | |
| 1171 | | | | | x | | x | | | | | x | | | | | | | | | | x | | | |
| 1172 | | | | | x | | x | | | | | x | | | | | | | | | | | x | | |
| 1173 | | | | | x | | x | | | | | x | | | | | | | | | | | | x | |
| 1174 | | | | | x | | x | | | | | x | | | | | | | | | | | | | x |
| 1175 | | | | | x | | x | | | | | | x | | | | x | | | | | | | | |
| 1176 | | | | | x | | x | | | | | | x | | | | | x | | | | | | | |
| 1177 | | | | | x | | x | | | | | | x | | | | | | x | | | | | | |
| 1178 | | | | | x | | x | | | | | | x | | | | | | | x | | | | | |
| 1179 | | | | | x | | x | | | | | | x | | | | | | | | x | | | | |
| 1180 | | | | | x | | x | | | | | | x | | | | | | | | | x | | | |
| 1181 | | | | | x | | x | | | | | | x | | | | | | | | | | | | |
| 1182 | | | | | x | | x | | | | | | x | | | | | | | | | | x | | |
| 1183 | | | | | x | | x | | | | | | x | | | | | | | | | | | x | |
| 1184 | | | | | x | | x | | | | | | x | | | | | | | | | | | | x |
| 1185 | | | | | x | | x | | | | | | | x | | | x | | | | | | | | |
| 1186 | | | | | x | | x | | | | | | | x | | | | x | | | | | | | |
| 1187 | | | | | x | | x | | | | | | | x | | | | | x | | | | | | |
| 1188 | | | | | x | | x | | | | | | | x | | | | | | x | | | | | |
| 1189 | | | | | x | | x | | | | | | | x | | | | | | | x | | | | |
| 1190 | | | | | x | | x | | | | | | | x | | | | | | | | x | | | |
| 1191 | | | | | x | | x | | | | | | | x | | | | | | | | | x | | |
| 1192 | | | | | x | | x | | | | | | | x | | | | | | | | | | x | |
| 1193 | | | | | x | | x | | | | | | | x | | | | | | | | | | | x |
| 1194 | | | | | x | | x | | | | | | | | x | | x | | | | | | | | |
| 1195 | | | | | x | | x | | | | | | | | x | | | x | | | | | | | |
| 1196 | | | | | x | | x | | | | | | | | x | | | | x | | | | | | |
| 1197 | | | | | x | | x | | | | | | | | x | | | | | x | | | | | |
| 1198 | | | | | x | | x | | | | | | | | x | | | | | | x | | | | |
| 1199 | | | | | x | | x | | | | | | | | x | | | | | | | x | | | |
| 1200 | | | | | x | | x | | | | | | | | x | | | | | | | | x | | |
| 1201 | | | | | x | | x | | | | | | | | x | | | | | | | | | x | |
| 1202 | | | | | x | | x | | | | | | | | x | | | | | | | | | | x |
| 1203 | | | | | x | | x | | | | | | | | | x | x | | | | | | | | |
| 1204 | | | | | x | | x | | | | | | | | | x | | x | | | | | | | |
| 1205 | | | | | x | | x | | | | | | | | | x | | | x | | | | | | |
| 1206 | | | | | x | | x | | | | | | | | | x | | | | x | | | | | |
| 1207 | | | | | x | | x | | | | | | | | | x | | | | | x | | | | |
| 1208 | | | | | x | | x | | | | | | | | | x | | | | | | x | | | |
| 1209 | | | | | x | | x | | | | | | | | | x | | | | | | | x | | |
| 1210 | | | | | x | | x | | | | | | | | | x | | | | | | | | x | |
| 1211 | | | | | x | | x | | | | | | | | | x | | | | | | | | | x |
| 1212 | | | | | x | | | x | | | x | | | | | | x | | | | | | | | |
| 1213 | | | | | x | | | x | | | x | | | | | | | x | | | | | | | |
| 1214 | | | | | x | | | x | | | x | | | | | | | | x | | | | | | |
| 1215 | | | | | x | | | x | | | x | | | | | | | | | x | | | | | |
| 1216 | | | | | x | | | x | | | x | | | | | | | | | | x | | | | |
| 1217 | | | | | x | | | x | | | x | | | | | | | | | | | x | | | |
| 1218 | | | | | x | | | x | | | x | | | | | | | | | | | | x | | |
| 1219 | | | | | x | | | x | | | x | | | | | | | | | | | | | x | |
| 1220 | | | | | x | | | x | | | x | | | | | | | | | | | | | | x |
| 1221 | | | | | x | | | x | | | | x | | | | | x | | | | | | | | |
| 1222 | | | | | x | | | x | | | | x | | | | | | x | | | | | | | |
| 1223 | | | | | x | | | x | | | | x | | | | | | | x | | | | | | |
| 1224 | | | | | x | | | x | | | | x | | | | | | | | x | | | | | |
| 1225 | | | | | x | | | x | | | | x | | | | | | | | | x | | | | |
| 1226 | | | | | x | | | x | | | | x | | | | | | | | | | x | | | |
| 1227 | | | | | x | | | x | | | | x | | | | | | | | | | | x | | |
| 1228 | | | | | x | | | x | | | | x | | | | | | | | | | | | x | |
| 1229 | | | | | x | | | x | | | | x | | | | | | | | | | | | | x |
| 1230 | | | | | x | | | x | | | | | x | | | | x | | | | | | | | |
| 1231 | | | | | x | | | x | | | | | x | | | | | x | | | | | | | |
| 1232 | | | | | x | | | x | | | | | x | | | | | | x | | | | | | |
| 1233 | | | | | x | | | x | | | | | x | | | | | | | x | | | | | |
| 1234 | | | | | x | | | x | | | | | x | | | | | | | | x | | | | |
| 1235 | | | | | x | | | x | | | | | x | | | | | | | | | x | | | |
| 1236 | | | | | x | | | x | | | | | x | | | | | | | | | | | | |
| 1237 | | | | | x | | | x | | | | | x | | | | | | | | | | x | | |
| 1238 | | | | | x | | | x | | | | | x | | | | | | | | | | | x | |
| 1239 | | | | | x | | | x | | | | | x | | | | | | | | | | | | x |
| 1240 | | | | | x | | | x | | | | | | x | | | x | | | | | | | | |
| 1241 | | | | | x | | | x | | | | | | x | | | | x | | | | | | | |
| 1242 | | | | | x | | | x | | | | | | x | | | | | x | | | | | | |
| 1243 | | | | | x | | | x | | | | | | x | | | | | | x | | | | | |
| 1244 | | | | | x | | | x | | | | | | x | | | | | | | x | | | | |
| 1245 | | | | | x | | | x | | | | | | x | | | | | | | | x | | | |
| 1246 | | | | | x | | | x | | | | | | x | | | | | | | | | x | | |
| 1247 | | | | | x | | | x | | | | | | x | | | | | | | | | | x | |
| 1248 | | | | | x | | | x | | | | | | x | | | | | | | | | | | x |
| 1249 | | | | | x | | | x | | | | | | | x | | x | | | | | | | | |
| 1250 | | | | | x | | | x | | | | | | | x | | | x | | | | | | | |
| 1251 | | | | | x | | | x | | | | | | | x | | | | x | | | | | | |
| 1252 | | | | | x | | | x | | | | | | | x | | | | | x | | | | | |
| 1253 | | | | | x | | | x | | | | | | | x | | | | | | x | | | | |
| 1254 | | | | | x | | | x | | | | | | | x | | | | | | | x | | | |
| 1255 | | | | | x | | | x | | | | | | | x | | | | | | | | x | | |
| 1256 | | | | | x | | | x | | | | | | | x | | | | | | | | | x | |
| 1257 | | | | | x | | | x | | | | | | | x | | | | | | | | | | x |
| 1258 | | | | | x | | | x | | | | | | | | x | x | | | | | | | | |
| 1259 | | | | | x | | | x | | | | | | | | x | | x | | | | | | | |
| 1260 | | | | | x | | | x | | | | | | | | x | | | x | | | | | | |
| 1261 | | | | | x | | | x | | | | | | | | x | | | | x | | | | | |
| 1262 | | | | | x | | | x | | | | | | | | x | | | | | x | | | | |
| 1263 | | | | | x | | | x | | | | | | | | x | | | | | | x | | | |
| 1264 | | | | | x | | | x | | | | | | | | x | | | | | | | x | | |
| 1265 | | | | | x | | | x | | | | | | | | x | | | | | | | | x | |
| 1266 | | | | | x | | | x | | | | | | | | x | | | | | | | | | x |
| 1267 | | | | | x | | | | x | | x | | | | | | x | | | | | | | | |
| 1268 | | | | | x | | | | x | | x | | | | | | | x | | | | | | | |
| 1269 | | | | | x | | | | x | | x | | | | | | | | x | | | | | | |
| 1270 | | | | | x | | | | x | | x | | | | | | | | | x | | | | | |
| 1271 | | | | | x | | | | x | | x | | | | | | | | | | x | | | | |
| 1272 | | | | | x | | | | x | | x | | | | | | | | | | | x | | | |
| 1273 | | | | | x | | | | x | | x | | | | | | | | | | | | x | | |
| 1274 | | | | | x | | | | x | | x | | | | | | | | | | | | | x | |
| 1275 | | | | | x | | | | x | | x | | | | | | | | | | | | | | x |
| 1276 | | | | | x | | | | x | | | x | | | | | x | | | | | | | | |
| 1277 | | | | | x | | | | x | | | x | | | | | | x | | | | | | | |
| 1278 | | | | | x | | | | x | | | x | | | | | | | x | | | | | | |
| 1279 | | | | | x | | | | x | | | x | | | | | | | | x | | | | | |
| 1280 | | | | | x | | | | x | | | x | | | | | | | | | x | | | | |
| 1281 | | | | | x | | | | x | | | x | | | | | | | | | | x | | | |
| 1282 | | | | | x | | | | x | | | x | | | | | | | | | | | x | | |
| 1283 | | | | | x | | | | x | | | x | | | | | | | | | | | | x | |
| 1284 | | | | | x | | | | x | | | x | | | | | | | | | | | | | x |
| 1285 | | | | | x | | | | x | | | | x | | | | x | | | | | | | | |
| 1286 | | | | | x | | | | x | | | | x | | | | | x | | | | | | | |
| 1287 | | | | | x | | | | x | | | | x | | | | | | x | | | | | | |
| 1288 | | | | | x | | | | x | | | | x | | | | | | | x | | | | | |
| 1289 | | | | | x | | | | x | | | | x | | | | | | | | x | | | | |
| 1290 | | | | | x | | | | x | | | | x | | | | | | | | | x | | | |
| 1291 | | | | | x | | | | x | | | | x | | | | | | | | | | | | |
| 1292 | | | | | x | | | | x | | | | x | | | | | | | | | | x | | |
| 1293 | | | | | x | | | | x | | | | x | | | | | | | | | | | x | |
| 1294 | | | | | x | | | | x | | | | x | | | | | | | | | | | | x |
| 1295 | | | | | x | | | | x | | | | | x | | | x | | | | | | | | |
| 1296 | | | | | x | | | | x | | | | | x | | | | x | | | | | | | |
| 1297 | | | | | x | | | | x | | | | | x | | | | | x | | | | | | |
| 1298 | | | | | x | | | | x | | | | | x | | | | | | x | | | | | |
| 1299 | | | | | x | | | | x | | | | | x | | | | | | | x | | | | |
| 1300 | | | | | x | | | | x | | | | | x | | | | | | | | x | | | |
| 1301 | | | | | x | | | | x | | | | | x | | | | | | | | | x | | |
| 1302 | | | | | x | | | | x | | | | | x | | | | | | | | | | x | |
| 1303 | | | | | x | | | | x | | | | | x | | | | | | | | | | | x |
| 1304 | | | | | x | | | | x | | | | | | x | | x | | | | | | | | |
| 1305 | | | | | x | | | | x | | | | | | x | | | x | | | | | | | |
| 1306 | | | | | x | | | | x | | | | | | x | | | | x | | | | | | |
| 1307 | | | | | x | | | | x | | | | | | x | | | | | x | | | | | |
| 1308 | | | | | x | | | | x | | | | | | x | | | | | | x | | | | |
| 1309 | | | | | x | | | | x | | | | | | x | | | | | | | x | | | |
| 1310 | | | | | x | | | | x | | | | | | x | | | | | | | | x | | |
| 1311 | | | | | x | | | | x | | | | | | x | | | | | | | | | x | |
| 1312 | | | | | x | | | | x | | | | | | x | | | | | | | | | | x |
| 1313 | | | | | x | | | | x | | | | | | | x | x | | | | | | | | |
| 1314 | | | | | x | | | | x | | | | | | | x | | x | | | | | | | |
| 1315 | | | | | x | | | | x | | | | | | | x | | | x | | | | | | |
| 1316 | | | | | x | | | | x | | | | | | | x | | | | x | | | | | |
| 1317 | | | | | x | | | | x | | | | | | | x | | | | | x | | | | |
| 1318 | | | | | x | | | | x | | | | | | | x | | | | | | x | | | |
| 1319 | | | | | x | | | | x | | | | | | | x | | | | | | | x | | |
| 1320 | | | | | x | | | | x | | | | | | | x | | | | | | | | x | |
| 1321 | | | | | x | | | | x | | | | | | | x | | | | | | | | | x |
| 1322 | | | | | x | | | | | x | x | | | | | | x | | | | | | | | |
| 1323 | | | | | x | | | | | x | x | | | | | | | x | | | | | | | |
| 1324 | | | | | x | | | | | x | x | | | | | | | | x | | | | | | |
| 1325 | | | | | x | | | | | x | x | | | | | | | | | x | | | | | |
| 1326 | | | | | x | | | | | x | x | | | | | | | | | | x | | | | |
| 1327 | | | | | x | | | | | x | x | | | | | | | | | | | x | | | |
| 1328 | | | | | x | | | | | x | x | | | | | | | | | | | | x | | |
| 1329 | | | | | x | | | | | x | x | | | | | | | | | | | | | x | |
| 1330 | | | | | x | | | | | x | x | | | | | | | | | | | | | | x |
| 1331 | | | | | x | | | | | x | | x | | | | | x | | | | | | | | |
| 1332 | | | | | x | | | | | x | | x | | | | | | x | | | | | | | |
| 1333 | | | | | x | | | | | x | | x | | | | | | | x | | | | | | |
| 1334 | | | | | x | | | | | x | | x | | | | | | | | x | | | | | |
| 1335 | | | | | x | | | | | x | | x | | | | | | | | | x | | | | |
| 1336 | | | | | x | | | | | x | | x | | | | | | | | | | x | | | |
| 1337 | | | | | x | | | | | x | | x | | | | | | | | | | | x | | |
| 1338 | | | | | x | | | | | x | | x | | | | | | | | | | | | x | |
| 1339 | | | | | x | | | | | x | | x | | | | | | | | | | | | | x |
| 1340 | | | | | x | | | | | x | | | x | | | | x | | | | | | | | |
| 1341 | | | | | x | | | | | x | | | x | | | | | x | | | | | | | |
| 1342 | | | | | x | | | | | x | | | x | | | | | | x | | | | | | |
| 1343 | | | | | x | | | | | x | | | x | | | | | | | x | | | | | |
| 1344 | | | | | x | | | | | x | | | x | | | | | | | | x | | | | |
| 1345 | | | | | x | | | | | x | | | x | | | | | | | | | x | | | |
| 1346 | | | | | x | | | | | x | | | x | | | | | | | | | | | | |
| 1347 | | | | | x | | | | | x | | | x | | | | | | | | | | x | | |
| 1348 | | | | | x | | | | | x | | | x | | | | | | | | | | | x | |
| 1349 | | | | | x | | | | | x | | | x | | | | | | | | | | | | x |
| 1350 | | | | | x | | | | | x | | | | x | | | x | | | | | | | | |
| 1351 | | | | | x | | | | | x | | | | x | | | | x | | | | | | | |
| 1352 | | | | | x | | | | | x | | | | x | | | | | x | | | | | | |
| 1353 | | | | | x | | | | | x | | | | x | | | | | | x | | | | | |
| 1354 | | | | | x | | | | | x | | | | x | | | | | | | x | | | | |
| 1355 | | | | | x | | | | | x | | | | x | | | | | | | | x | | | |
| 1356 | | | | | x | | | | | x | | | | x | | | | | | | | | x | | |
| 1357 | | | | | x | | | | | x | | | | x | | | | | | | | | | x | |
| 1358 | | | | | x | | | | | x | | | | x | | | | | | | | | | | x |
| 1359 | | | | | x | | | | | x | | | | | x | | x | | | | | | | | |
| 1360 | | | | | x | | | | | x | | | | | x | | | x | | | | | | | |
| 1361 | | | | | x | | | | | x | | | | | x | | | | x | | | | | | |
| 1362 | | | | | x | | | | | x | | | | | x | | | | | x | | | | | |
| 1363 | | | | | x | | | | | x | | | | | x | | | | | | x | | | | |
| 1364 | | | | | x | | | | | x | | | | | x | | | | | | | x | | | |
| 1365 | | | | | x | | | | | x | | | | | x | | | | | | | | x | | |
| 1366 | | | | | x | | | | | x | | | | | x | | | | | | | | | x | |
| 1367 | | | | | x | | | | | x | | | | | x | | | | | | | | | | x |
| 1368 | | | | | x | | | | | x | | | | | | x | x | | | | | | | | |
| 1369 | | | | | x | | | | | x | | | | | | x | | x | | | | | | | |
| 1370 | | | | | x | | | | | x | | | | | | x | | | x | | | | | | |
| 1371 | | | | | x | | | | | x | | | | | | x | | | | x | | | | | |
| 1372 | | | | | x | | | | | x | | | | | | x | | | | | x | | | | |
| 1373 | | | | | x | | | | | x | | | | | | x | | | | | | x | | | |
| 1374 | | | | | x | | | | | x | | | | | | x | | | | | | | x | | |
| 1375 | | | | | x | | | | | x | | | | | | x | | | | | | | | x | |
| 1376 | | | | | x | | | | | x | | | | | | x | | | | | | | | | x |

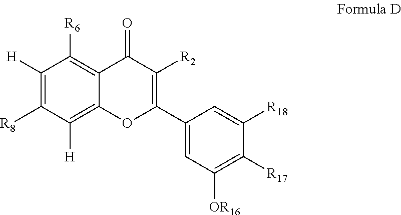

Key: Substituents described in Table 2 of exemplary compounds of Formula D are abbreviated as shown herein.

| | |
|---|---|
| E = -Oglucoronide | G = -glucoronide |
| J = —PO₃T | K = —OPO₃T |
| V = —CH₂OPO₃T | L = —OCH₂OPO₃T |

TABLE A
Exemplary formulas of T as described in the Exemplary compounds of
Formula D as disclosed in Table 2.

| Subclass # | T | Subclass # | T |
|---|---|---|---|
| S-1 | H, H | S-2 | H, K |
| S-3 | K, K | S-4 | H, Li |
| S-5 | Li, Li | S-6 | H, Na |
| S-7 | Na, Na | S-8 | H, Me |
| S-9 | Me, Me | S-10 | H, ethyl |
| S-11 | ethyl, ethyl | S-12 | H, glucose |
| S-13 | Glucose, glucose | S-14 | Ca |
| S-15 | Mg | S-16 | Fe(II) |
| S-17 | Zn | | |

TABLE 2
Exemplary compounds of Formula D. Each subclass in Table 2, utilizes each subclass of “T” as described in Table A.

| Sub- | | | | | | | | | | | | | | | | | | | | | | | |
|---|---|---|---|---|---|---|---|---|---|---|---|---|---|---|---|---|---|---|---|---|---|---|---|
| class # | R₂ | | | | | R₆ | | R₈ | | | | | | R₁₆ | | | | | R₁₇ | | | R₁₈ | |
| 1 | H | OH | E | K | L | H | OH | H | F | E | K | L | Me | H | Me | G | J | V | H | OMe | K | H | OH |
| 2 | x | | | | | x | | x | | | | | | x | | | | | x | | | x | |
| 3 | x | | | | | x | | x | | | | | | x | | | | | x | | | | x |
| 4 | x | | | | | x | | x | | | | | | x | | | | | | x | | x | |
| 5 | x | | | | | x | | x | | | | | | x | | | | | | x | | | x |
| 6 | x | | | | | x | | x | | | | | | x | | | | | | | x | x | |
| 7 | x | | | | | x | | x | | | | | | x | | | | | | | x | | x |
| 8 | x | | | | | x | | x | | | | | | | x | | | | x | | | x | |
| 9 | x | | | | | x | | x | | | | | | | x | | | | x | | | | x |
| 10 | x | | | | | x | | x | | | | | | | x | | | | | x | | x | |
| 11 | x | | | | | x | | x | | | | | | | x | | | | | x | | | x |
| 12 | x | | | | | x | | x | | | | | | | x | | | | | | x | x | |
| 13 | x | | | | | x | | x | | | | | | | x | | | | | | x | | x |
| 14 | x | | | | | x | | x | | | | | | | | x | | | x | | | x | |
| 15 | x | | | | | x | | x | | | | | | | | x | | | x | | | | x |
| 16 | x | | | | | x | | x | | | | | | | | x | | | | x | | x | |
| 17 | x | | | | | x | | x | | | | | | | | x | | | | x | | | x |
| 18 | x | | | | | x | | x | | | | | | | | x | | | | | x | x | |
| 19 | x | | | | | x | | x | | | | | | | | x | | | | | x | | x |
| 20 | x | | | | | x | | x | | | | | | | | | x | | x | | | x | |
| 21 | x | | | | | x | | x | | | | | | | | | x | | x | | | | x |
| 22 | x | | | | | x | | x | | | | | | | | | x | | | x | | x | |
| 23 | x | | | | | x | | x | | | | | | | | | x | | | x | | | x |
| 24 | x | | | | | x | | x | | | | | | | | | x | | | | x | x | |
| 25 | x | | | | | x | | x | | | | | | | | | x | | | | x | | x |
| 26 | x | | | | | x | | x | | | | | | | | | | x | x | | | x | |
| 27 | x | | | | | x | | x | | | | | | | | | | x | x | | | | x |
| 28 | x | | | | | x | | x | | | | | | | | | | x | | x | | x | |
| 29 | x | | | | | x | | x | | | | | | | | | | x | | x | | | x |
| 30 | x | | | | | x | | x | | | | | | | | | | x | | | x | x | |
| 31 | x | | | | | x | | x | | | | | | | | | | x | | | x | | x |
| 32 | x | | | | | x | | | x | | | | | x | | | | | x | | | x | |
| 33 | x | | | | | x | | | x | | | | | x | | | | | x | | | | x |
| 34 | x | | | | | x | | | x | | | | | x | | | | | | x | | x | |
| 35 | x | | | | | x | | | x | | | | | x | | | | | | x | | | x |
| 36 | x | | | | | x | | | x | | | | | x | | | | | | | x | x | |
| 37 | x | | | | | x | | | x | | | | | x | | | | | | | x | | x |
| 38 | x | | | | | x | | | x | | | | | | x | | | | x | | | x | |
| 39 | x | | | | | x | | | x | | | | | | x | | | | x | | | | x |
| 40 | x | | | | | x | | | x | | | | | | x | | | | | x | | x | |
| 41 | x | | | | | x | | | x | | | | | | x | | | | | x | | | x |
| 42 | x | | | | | x | | | x | | | | | | x | | | | | | x | x | |
| 43 | x | | | | | x | | | x | | | | | | x | | | | | | x | | x |
| 44 | x | | | | | x | | | x | | | | | | | x | | | x | | | x | |
| 45 | x | | | | | x | | | x | | | | | | | x | | | x | | | | x |
| 46 | x | | | | | x | | | x | | | | | | | x | | | | x | | x | |
| 47 | x | | | | | x | | | x | | | | | | | x | | | | x | | | x |
| 48 | x | | | | | x | | | x | | | | | | | x | | | | | x | x | |
| 49 | x | | | | | x | | | x | | | | | | | x | | | | | x | | x |
| 50 | x | | | | | x | | | x | | | | | | | | x | | x | | | x | |
| 51 | x | | | | | x | | | x | | | | | | | | x | | x | | | | x |
| 52 | x | | | | | x | | | x | | | | | | | | x | | | x | | x | |
| 53 | x | | | | | x | | | x | | | | | | | | x | | | x | | | x |
| 54 | x | | | | | x | | | x | | | | | | | | x | | | | x | x | |
| 55 | x | | | | | x | | | x | | | | | | | | x | | | | x | | x |
| 56 | x | | | | | x | | | x | | | | | | | | | x | x | | | x | |
| 57 | x | | | | | x | | | x | | | | | | | | | x | x | | | | x |
| 58 | x | | | | | x | | | x | | | | | | | | | x | | x | | x | |
| 59 | x | | | | | x | | | x | | | | | | | | | x | | x | | | x |
| 60 | x | | | | | x | | | x | | | | | | | | | x | | | x | x | |
| 61 | x | | | | | x | | | x | | | | | | | | | x | | | x | | x |
| 62 | x | | | | | x | | | | x | | | | x | | | | | x | | | x | |
| 63 | x | | | | | x | | | | x | | | | x | | | | | x | | | | x |
| 64 | x | | | | | x | | | | x | | | | x | | | | | | x | | x | |
| 65 | x | | | | | x | | | | x | | | | x | | | | | | x | | | x |
| 66 | x | | | | | x | | | | x | | | | x | | | | | | | x | x | |
| 67 | x | | | | | x | | | | x | | | | x | | | | | | | x | | x |
| 68 | x | | | | | x | | | | x | | | | | x | | | | x | | | x | |
| 69 | x | | | | | x | | | | x | | | | | x | | | | x | | | | x |
| 70 | x | | | | | x | | | | x | | | | | x | | | | | x | | x | |
| 71 | x | | | | | x | | | | x | | | | | x | | | | | x | | | x |
| 72 | x | | | | | x | | | | x | | | | | x | | | | | | x | x | |
| 73 | x | | | | | x | | | | x | | | | | x | | | | | | x | | x |
| 74 | x | | | | | x | | | | x | | | | | | x | | | x | | | x | |
| 75 | x | | | | | x | | | | x | | | | | | x | | | x | | | | x |
| 76 | x | | | | | x | | | | x | | | | | | x | | | | x | | x | |
| 77 | x | | | | | x | | | | x | | | | | | x | | | | x | | | x |
| 78 | x | | | | | x | | | | x | | | | | | x | | | | | x | x | |
| 79 | x | | | | | x | | | | x | | | | | | x | | | | | x | | x |
| 80 | x | | | | | x | | | | x | | | | | | | x | | x | | | x | |
| 81 | x | | | | | x | | | | x | | | | | | | x | | x | | | | x |
| 82 | x | | | | | x | | | | x | | | | | | | x | | | x | | x | |
| 83 | x | | | | | x | | | | x | | | | | | | x | | | x | | | x |
| 84 | x | | | | | x | | | | x | | | | | | | x | | | | x | x | |
| 85 | x | | | | | x | | | | x | | | | | | | x | | | | x | | x |
| 86 | x | | | | | x | | | | x | | | | | | | | x | x | | | x | |
| 87 | x | | | | | x | | | | x | | | | | | | | x | x | | | | x |
| 88 | x | | | | | x | | | | x | | | | | | | | x | | x | | x | |
| 89 | x | | | | | x | | | | x | | | | | | | | x | | x | | | x |
| 90 | x | | | | | x | | | | x | | | | | | | | x | | | x | x | |
| 91 | x | | | | | x | | | | x | | | | | | | | x | | | x | | x |
| 92 | x | | | | | x | | | | | x | | | x | | | | | x | | | x | |
| 93 | x | | | | | x | | | | | x | | | x | | | | | x | | | | x |
| 94 | x | | | | | x | | | | | x | | | x | | | | | | x | | x | |
| 95 | x | | | | | x | | | | | x | | | x | | | | | | x | | | x |
| 96 | x | | | | | x | | | | | x | | | x | | | | | | | x | x | |
| 97 | x | | | | | x | | | | | x | | | x | | | | | | | x | | x |
| 98 | x | | | | | x | | | | | x | | | | x | | | | x | | | x | |
| 99 | x | | | | | x | | | | | x | | | | x | | | | x | | | | x |
| 100 | x | | | | | x | | | | | x | | | | x | | | | | x | | x | |
| 101 | x | | | | | x | | | | | x | | | | x | | | | | x | | | x |
| 102 | x | | | | | x | | | | | x | | | | x | | | | | | x | x | |
| 103 | x | | | | | x | | | | | x | | | | x | | | | | | x | | x |
| 104 | x | | | | | x | | | | | x | | | | | x | | | x | | | x | |
| 105 | x | | | | | x | | | | | x | | | | | x | | | x | | | | x |
| 106 | x | | | | | x | | | | | x | | | | | x | | | | x | | x | |
| 107 | x | | | | | x | | | | | x | | | | | x | | | | x | | | x |
| 108 | x | | | | | x | | | | | x | | | | | x | | | | | x | x | |
| 109 | x | | | | | x | | | | | x | | | | | x | | | | | x | | x |
| 110 | x | | | | | x | | | | | x | | | | | | x | | x | | | x | |
| 111 | x | | | | | x | | | | | x | | | | | | x | | x | | | | x |
| 112 | x | | | | | x | | | | | x | | | | | | x | | | x | | x | |
| 113 | x | | | | | x | | | | | x | | | | | | x | | | x | | | x |
| 114 | x | | | | | x | | | | | x | | | | | | x | | | | x | x | |
| 115 | x | | | | | x | | | | | x | | | | | | x | | | | x | | x |
| 116 | x | | | | | x | | | | | x | | | | | | | x | x | | | x | |
| 117 | x | | | | | x | | | | | x | | | | | | | x | x | | | | x |
| 118 | x | | | | | x | | | | | x | | | | | | | x | | x | | x | |
| 119 | x | | | | | x | | | | | x | | | | | | | x | | x | | | x |
| 120 | x | | | | | x | | | | | x | | | | | | | x | | | x | x | |
| 121 | x | | | | | x | | | | | x | | | | | | | x | | | x | | x |
| 122 | x | | | | | x | | | | | | x | | x | | | | | x | | | x | |
| 123 | x | | | | | x | | | | | | x | | x | | | | | x | | | | x |
| 124 | x | | | | | x | | | | | | x | | x | | | | | | x | | x | |
| 125 | x | | | | | x | | | | | | x | | x | | | | | | x | | | x |
| 126 | x | | | | | x | | | | | | x | | x | | | | | | | x | x | |
| 127 | x | | | | | x | | | | | | x | | x | | | | | | | x | | x |
| 128 | x | | | | | x | | | | | | x | | | x | | | | x | | | x | |
| 129 | x | | | | | x | | | | | | x | | | x | | | | x | | | | x |
| 130 | x | | | | | x | | | | | | x | | | x | | | | | x | | x | |
| 131 | x | | | | | x | | | | | | x | | | x | | | | | x | | | x |
| 132 | x | | | | | x | | | | | | x | | | x | | | | | | x | x | |
| 133 | x | | | | | x | | | | | | x | | | x | | | | | | x | | x |
| 134 | x | | | | | x | | | | | | x | | | | x | | | x | | | x | |
| 135 | x | | | | | x | | | | | | x | | | | x | | | x | | | | x |
| 136 | x | | | | | x | | | | | | x | | | | x | | | | x | | x | |
| 137 | x | | | | | x | | | | | | x | | | | x | | | | x | | | x |
| 138 | x | | | | | x | | | | | | x | | | | x | | | | | x | x | |
| 139 | x | | | | | x | | | | | | x | | | | x | | | | | x | | x |
| 140 | x | | | | | x | | | | | | x | | | | | x | | x | | | x | |
| 141 | x | | | | | x | | | | | | x | | | | | x | | x | | | | x |
| 142 | x | | | | | x | | | | | | x | | | | | x | | | x | | x | |
| 143 | x | | | | | x | | | | | | x | | | | | x | | | x | | | x |
| 144 | x | | | | | x | | | | | | x | | | | | x | | | | x | x | |
| 145 | x | | | | | x | | | | | | x | | | | | x | | | | x | | x |
| 146 | x | | | | | x | | | | | | x | | | | | | x | x | | | x | |
| 147 | x | | | | | x | | | | | | x | | | | | | x | x | | | | x |
| 148 | x | | | | | x | | | | | | x | | | | | | x | | x | | x | |
| 149 | x | | | | | x | | | | | | x | | | | | | x | | x | | | x |
| 150 | x | | | | | x | | | | | | x | | | | | | x | | | x | x | |
| 151 | x | | | | | x | | | | | | x | | | | | | x | | | x | | x |
| 152 | x | | | | | x | | | | | | | x | x | | | | | x | | | x | |
| 153 | x | | | | | x | | | | | | | x | x | | | | | x | | | | x |
| 154 | x | | | | | x | | | | | | | x | x | | | | | | x | | x | |
| 155 | x | | | | | x | | | | | | | x | x | | | | | | x | | | x |
| 156 | x | | | | | x | | | | | | | x | x | | | | | | | x | x | |
| 157 | x | | | | | x | | | | | | | x | x | | | | | | | x | | x |
| 158 | x | | | | | x | | | | | | | x | | x | | | | x | | | x | |
| 159 | x | | | | | x | | | | | | | x | | x | | | | x | | | | x |
| 160 | x | | | | | x | | | | | | | x | | x | | | | | x | | x | |
| 161 | x | | | | | x | | | | | | | x | | x | | | | | x | | | x |
| 162 | x | | | | | x | | | | | | | x | | x | | | | | | x | x | |
| 163 | x | | | | | x | | | | | | | x | | x | | | | | | x | | x |
| 164 | x | | | | | x | | | | | | | x | | | x | | | x | | | x | |
| 165 | x | | | | | x | | | | | | | x | | | x | | | x | | | | x |
| 166 | x | | | | | x | | | | | | | x | | | x | | | | x | | x | |
| 167 | x | | | | | x | | | | | | | x | | | x | | | | x | | | x |
| 168 | x | | | | | x | | | | | | | x | | | x | | | | | x | x | |
| 169 | x | | | | | x | | | | | | | x | | | x | | | | | x | | x |
| 170 | x | | | | | x | | | | | | | x | | | | x | | x | | | x | |
| 171 | x | | | | | x | | | | | | | x | | | | x | | x | | | | x |
| 172 | x | | | | | x | | | | | | | x | | | | x | | | x | | x | |
| 173 | x | | | | | x | | | | | | | x | | | | x | | | x | | | x |
| 174 | x | | | | | x | | | | | | | x | | | | x | | | | x | x | |
| 175 | x | | | | | x | | | | | | | x | | | | x | | | | x | | x |
| 176 | x | | | | | x | | | | | | | x | | | | | x | x | | | x | |
| 177 | x | | | | | x | | | | | | | x | | | | | x | x | | | | x |
| 178 | x | | | | | x | | | | | | | x | | | | | x | | x | | x | |
| 179 | x | | | | | x | | | | | | | x | | | | | x | | x | | | x |
| 180 | x | | | | | x | | | | | | | x | | | | | x | | | x | x | |
| 181 | x | | | | | x | | | | | | | x | | | | | x | | | x | | x |
| 182 | x | | | | | | x | x | | | | | | x | | | | | x | | | x | |
| 183 | x | | | | | | x | x | | | | | | x | | | | | x | | | | x |
| 184 | x | | | | | | x | x | | | | | | x | | | | | | x | | x | |
| 185 | x | | | | | | x | x | | | | | | x | | | | | | x | | | x |
| 186 | x | | | | | | x | x | | | | | | x | | | | | | | x | x | |
| 187 | x | | | | | | x | x | | | | | | x | | | | | | | x | | x |
| 188 | x | | | | | | x | x | | | | | | | x | | | | x | | | x | |
| 189 | x | | | | | | x | x | | | | | | | x | | | | x | | | | x |
| 190 | x | | | | | | x | x | | | | | | | x | | | | | x | | x | |
| 191 | x | | | | | | x | x | | | | | | | x | | | | | x | | | x |
| 192 | x | | | | | | x | x | | | | | | | x | | | | | | x | x | |
| 193 | x | | | | | | x | x | | | | | | | x | | | | | | x | | x |
| 194 | x | | | | | | x | x | | | | | | | | x | | | x | | | x | |
| 195 | x | | | | | | x | x | | | | | | | | x | | | x | | | | x |
| 196 | x | | | | | | x | x | | | | | | | | x | | | | x | | x | |
| 197 | x | | | | | | x | x | | | | | | | | x | | | | x | | | x |
| 198 | x | | | | | | x | x | | | | | | | | x | | | | | x | x | |
| 199 | x | | | | | | x | x | | | | | | | | x | | | | | x | | x |
| 200 | x | | | | | | x | x | | | | | | | | | x | | x | | | x | |
| 201 | x | | | | | | x | x | | | | | | | | | x | | x | | | | x |
| 202 | x | | | | | | x | x | | | | | | | | | x | | | x | | x | |
| 203 | x | | | | | | x | x | | | | | | | | | x | | | x | | | x |
| 204 | x | | | | | | x | x | | | | | | | | | x | | | | x | x | |
| 205 | x | | | | | | x | x | | | | | | | | | x | | | | x | | x |
| 206 | x | | | | | | x | x | | | | | | | | | | x | x | | | x | |
| 207 | x | | | | | | x | x | | | | | | | | | | x | x | | | | x |
| 208 | x | | | | | | x | x | | | | | | | | | | x | | x | | x | |
| 209 | x | | | | | | x | x | | | | | | | | | | x | | x | | | x |
| 210 | x | | | | | | x | x | | | | | | | | | | x | | | x | x | |
| 211 | x | | | | | | x | x | | | | | | | | | | x | | | x | | x |
| 212 | x | | | | | | x | | x | | | | | x | | | | | x | | | x | |
| 213 | x | | | | | | x | | x | | | | | x | | | | | x | | | | x |
| 214 | x | | | | | | x | | x | | | | | x | | | | | | x | | x | |
| 215 | x | | | | | | x | | x | | | | | x | | | | | | x | | | x |
| 216 | x | | | | | | x | | x | | | | | x | | | | | | | x | x | |
| 217 | x | | | | | | x | | x | | | | | x | | | | | | | x | | x |
| 218 | x | | | | | | x | | x | | | | | | x | | | | x | | | x | |
| 219 | x | | | | | | x | | x | | | | | | x | | | | x | | | | x |
| 220 | x | | | | | | x | | x | | | | | | x | | | | | x | | x | |
| 221 | x | | | | | | x | | x | | | | | | x | | | | | x | | | x |
| 222 | x | | | | | | x | | x | | | | | | x | | | | | | x | x | |
| 223 | x | | | | | | x | | x | | | | | | x | | | | | | x | | x |
| 224 | x | | | | | | x | | x | | | | | | | x | | | x | | | x | |
| 225 | x | | | | | | x | | x | | | | | | | x | | | x | | | | x |
| 226 | x | | | | | | x | | x | | | | | | | x | | | | x | | x | |
| 227 | x | | | | | | x | | x | | | | | | | x | | | | x | | | x |
| 228 | x | | | | | | x | | x | | | | | | | x | | | | | x | x | |
| 229 | x | | | | | | x | | x | | | | | | | x | | | | | x | | x |
| 230 | x | | | | | | x | | x | | | | | | | | x | | x | | | x | |
| 231 | x | | | | | | x | | x | | | | | | | | x | | x | | | | x |
| 232 | x | | | | | | x | | x | | | | | | | | x | | | x | | x | |
| 233 | x | | | | | | x | | x | | | | | | | | x | | | x | | | x |
| 234 | x | | | | | | x | | x | | | | | | | | x | | | | x | x | |
| 235 | x | | | | | | x | | x | | | | | | | | x | | | | x | | x |
| 236 | x | | | | | | x | | x | | | | | | | | | x | x | | | x | |
| 237 | x | | | | | | x | | x | | | | | | | | | x | x | | | | x |
| 238 | x | | | | | | x | | x | | | | | | | | | x | | x | | x | |
| 239 | x | | | | | | x | | x | | | | | | | | | x | | x | | | x |
| 240 | x | | | | | | x | | x | | | | | | | | | x | | | x | x | |
| 241 | x | | | | | | x | | x | | | | | | | | | x | | | x | | x |
| 242 | x | | | | | | x | | | x | | | | x | | | | | x | | | x | |
| 243 | x | | | | | | x | | | x | | | | x | | | | | x | | | | x |
| 244 | x | | | | | | x | | | x | | | | x | | | | | | x | | x | |
| 245 | x | | | | | | x | | | x | | | | x | | | | | | x | | | x |
| 246 | x | | | | | | x | | | x | | | | x | | | | | | | x | x | |
| 247 | x | | | | | | x | | | x | | | | x | | | | | | | x | | x |
| 248 | x | | | | | | x | | | x | | | | | x | | | | x | | | x | |
| 249 | x | | | | | | x | | | x | | | | | x | | | | x | | | | x |
| 250 | x | | | | | | x | | | x | | | | | x | | | | | x | | x | |
| 251 | x | | | | | | x | | | x | | | | | x | | | | | x | | | x |
| 252 | x | | | | | | x | | | x | | | | | x | | | | | | x | x | |
| 253 | x | | | | | | x | | | x | | | | | x | | | | | | x | | x |
| 254 | x | | | | | | x | | | x | | | | | | x | | | x | | | x | |
| 255 | x | | | | | | x | | | x | | | | | | x | | | x | | | | x |
| 256 | x | | | | | | x | | | x | | | | | | x | | | | x | | x | |
| 257 | x | | | | | | x | | | x | | | | | | x | | | | x | | | x |
| 258 | x | | | | | | x | | | x | | | | | | x | | | | | x | x | |
| 259 | x | | | | | | x | | | x | | | | | | x | | | | | x | | x |
| 260 | x | | | | | | x | | | x | | | | | | | x | | x | | | x | |
| 261 | x | | | | | | x | | | x | | | | | | | x | | x | | | | x |
| 262 | x | | | | | | x | | | x | | | | | | | x | | | x | | x | |
| 263 | x | | | | | | x | | | x | | | | | | | x | | | x | | | x |
| 264 | x | | | | | | x | | | x | | | | | | | x | | | | x | x | |
| 265 | x | | | | | | x | | | x | | | | | | | x | | | | x | | x |
| 266 | x | | | | | | x | | | x | | | | | | | | x | x | | | x | |
| 267 | x | | | | | | x | | | x | | | | | | | | x | x | | | | x |
| 268 | x | | | | | | x | | | x | | | | | | | | x | | x | | x | |
| 269 | x | | | | | | x | | | x | | | | | | | | x | | x | | | x |
| 270 | x | | | | | | x | | | x | | | | | | | | x | | | x | x | |
| 271 | x | | | | | | x | | | x | | | | | | | | x | | | x | | x |
| 272 | x | | | | | | x | | | | x | | | x | | | | | x | | | x | |
| 273 | x | | | | | | x | | | | x | | | x | | | | | x | | | | x |
| 274 | x | | | | | | x | | | | x | | | x | | | | | | x | | x | |
| 275 | x | | | | | | x | | | | x | | | x | | | | | | x | | | x |
| 276 | x | | | | | | x | | | | x | | | x | | | | | | | x | x | |
| 277 | x | | | | | | x | | | | x | | | x | | | | | | | x | | x |
| 278 | x | | | | | | x | | | | x | | | | x | | | | x | | | x | |
| 279 | x | | | | | | x | | | | x | | | | x | | | | x | | | | x |
| 280 | x | | | | | | x | | | | x | | | | x | | | | | x | | x | |
| 281 | x | | | | | | x | | | | x | | | | x | | | | | x | | | x |
| 282 | x | | | | | | x | | | | x | | | | x | | | | | | x | x | |
| 283 | x | | | | | | x | | | | x | | | | x | | | | | | x | | x |
| 284 | x | | | | | | x | | | | x | | | | | x | | | x | | | x | |
| 285 | x | | | | | | x | | | | x | | | | | x | | | x | | | | x |
| 286 | x | | | | | | x | | | | x | | | | | x | | | | x | | x | |
| 287 | x | | | | | | x | | | | x | | | | | x | | | | x | | | x |
| 288 | x | | | | | | x | | | | x | | | | | x | | | | | x | x | |
| 289 | x | | | | | | x | | | | x | | | | | x | | | | | x | | x |
| 290 | x | | | | | | x | | | | x | | | | | | x | | x | | | x | |
| 291 | x | | | | | | x | | | | x | | | | | | x | | x | | | | x |
| 292 | x | | | | | | x | | | | x | | | | | | x | | | x | | x | |
| 293 | x | | | | | | x | | | | x | | | | | | x | | | x | | | x |
| 294 | x | | | | | | x | | | | x | | | | | | x | | | | x | x | |
| 295 | x | | | | | | x | | | | x | | | | | | x | | | | x | | x |
| 296 | x | | | | | | x | | | | x | | | | | | | x | x | | | x | |
| 297 | x | | | | | | x | | | | x | | | | | | | x | x | | | | x |
| 298 | x | | | | | | x | | | | x | | | | | | | x | | x | | x | |
| 299 | x | | | | | | x | | | | x | | | | | | | x | | x | | | x |
| 300 | x | | | | | | x | | | | x | | | | | | | x | | | x | x | |
| 301 | x | | | | | | x | | | | x | | | | | | | x | | | x | | x |
| 302 | x | | | | | | x | | | | | x | | x | | | | | x | | | x | |
| 303 | x | | | | | | x | | | | | x | | x | | | | | x | | | | x |
| 304 | x | | | | | | x | | | | | x | | x | | | | | | x | | x | |
| 305 | x | | | | | | x | | | | | x | | x | | | | | | x | | | x |
| 306 | x | | | | | | x | | | | | x | | x | | | | | | | x | x | |
| 307 | x | | | | | | x | | | | | x | | x | | | | | | | x | | x |
| 308 | x | | | | | | x | | | | | x | | | x | | | | x | | | x | |
| 309 | x | | | | | | x | | | | | x | | | x | | | | x | | | | x |
| 310 | x | | | | | | x | | | | | x | | | x | | | | | x | | x | |
| 311 | x | | | | | | x | | | | | x | | | x | | | | | x | | | x |
| 312 | x | | | | | | x | | | | | x | | | x | | | | | | x | x | |
| 313 | x | | | | | | x | | | | | x | | | x | | | | | | x | | x |
| 314 | x | | | | | | x | | | | | x | | | | x | | | x | | | x | |
| 315 | x | | | | | | x | | | | | x | | | | x | | | x | | | | x |
| 316 | x | | | | | | x | | | | | x | | | | x | | | | x | | x | |
| 317 | x | | | | | | x | | | | | x | | | | x | | | | x | | | x |
| 318 | x | | | | | | x | | | | | x | | | | x | | | | | x | x | |
| 319 | x | | | | | | x | | | | | x | | | | x | | | | | x | | x |
| 320 | x | | | | | | x | | | | | x | | | | | x | | x | | | x | |
| 321 | x | | | | | | x | | | | | x | | | | | x | | x | | | | x |
| 322 | x | | | | | | x | | | | | x | | | | | x | | | x | | x | |
| 323 | x | | | | | | x | | | | | x | | | | | x | | | x | | | x |
| 324 | x | | | | | | x | | | | | x | | | | | x | | | | x | x | |
| 325 | x | | | | | | x | | | | | x | | | | | x | | | | x | | x |
| 326 | x | | | | | | x | | | | | x | | | | | | x | x | | | x | |
| 327 | x | | | | | | x | | | | | x | | | | | | x | x | | | | x |
| 328 | x | | | | | | x | | | | | x | | | | | | x | | x | | x | |
| 329 | x | | | | | | x | | | | | x | | | | | | x | | x | | | x |
| 330 | x | | | | | | x | | | | | x | | | | | | x | | | x | x | |
| 331 | x | | | | | | x | | | | | x | | | | | | x | | | x | | x |
| 332 | x | | | | | | x | | | | | | x | x | | | | | x | | | x | |
| 333 | x | | | | | | x | | | | | | x | x | | | | | x | | | | x |
| 334 | x | | | | | | x | | | | | | x | x | | | | | | x | | x | |
| 335 | x | | | | | | x | | | | | | x | x | | | | | | x | | | x |
| 336 | x | | | | | | x | | | | | | x | x | | | | | | | x | x | |
| 337 | x | | | | | | x | | | | | | x | x | | | | | | | x | | x |
| 338 | x | | | | | | x | | | | | | x | | x | | | | x | | | x | |
| 339 | x | | | | | | x | | | | | | x | | x | | | | x | | | | x |
| 340 | x | | | | | | x | | | | | | x | | x | | | | | x | | x | |
| 341 | x | | | | | | x | | | | | | x | | x | | | | | x | | | x |
| 342 | x | | | | | | x | | | | | | x | | x | | | | | | x | x | |
| 343 | x | | | | | | x | | | | | | x | | x | | | | | | x | | x |
| 344 | x | | | | | | x | | | | | | x | | | x | | | x | | | x | |
| 345 | x | | | | | | x | | | | | | x | | | x | | | x | | | | x |
| 346 | x | | | | | | x | | | | | | x | | | x | | | | x | | x | |
| 347 | x | | | | | | x | | | | | | x | | | x | | | | x | | | x |
| 348 | x | | | | | | x | | | | | | x | | | x | | | | | x | x | |
| 349 | x | | | | | | x | | | | | | x | | | x | | | | | x | | x |
| 350 | x | | | | | | x | | | | | | x | | | | x | | x | | | x | |
| 351 | x | | | | | | x | | | | | | x | | | | x | | x | | | | x |
| 352 | x | | | | | | x | | | | | | x | | | | x | | | x | | x | |
| 353 | x | | | | | | x | | | | | | x | | | | x | | | x | | | x |
| 354 | x | | | | | | x | | | | | | x | | | | x | | | | x | x | |
| 355 | x | | | | | | x | | | | | | x | | | | x | | | | x | | x |
| 356 | x | | | | | | x | | | | | | x | | | | | x | x | | | x | |
| 357 | x | | | | | | x | | | | | | x | | | | | x | x | | | | x |
| 358 | x | | | | | | x | | | | | | x | | | | | x | | x | | x | |
| 359 | x | | | | | | x | | | | | | x | | | | | x | | x | | | x |
| 360 | x | | | | | | x | | | | | | x | | | | | x | | | x | x | |
| 361 | x | | | | | | x | | | | | | x | | | | | x | | | x | | x |
| 362 | | x | | | | x | | x | | | | | | x | | | | | x | | | x | |
| 363 | | x | | | | x | | x | | | | | | x | | | | | x | | | | x |
| 364 | | x | | | | x | | x | | | | | | x | | | | | | x | | x | |
| 365 | | x | | | | x | | x | | | | | | x | | | | | | x | | | x |
| 366 | | x | | | | x | | x | | | | | | x | | | | | | | x | x | |
| 367 | | x | | | | x | | x | | | | | | x | | | | | | | x | | x |
| 368 | | x | | | | x | | x | | | | | | | x | | | | x | | | x | |
| 369 | | x | | | | x | | x | | | | | | | x | | | | x | | | | x |
| 370 | | x | | | | x | | x | | | | | | | x | | | | | x | | x | |
| 371 | | x | | | | x | | x | | | | | | | x | | | | | x | | | x |
| 372 | | x | | | | x | | x | | | | | | | x | | | | | | x | x | |
| 373 | | x | | | | x | | x | | | | | | | x | | | | | | x | | x |
| 374 | | x | | | | x | | x | | | | | | | | x | | | x | | | x | |
| 375 | | x | | | | x | | x | | | | | | | | x | | | x | | | | x |
| 376 | | x | | | | x | | x | | | | | | | | x | | | | x | | x | |
| 377 | | x | | | | x | | x | | | | | | | | x | | | | x | | | x |
| 378 | | x | | | | x | | x | | | | | | | | x | | | | | x | x | |
| 379 | | x | | | | x | | x | | | | | | | | x | | | | | x | | x |
| 380 | | x | | | | x | | x | | | | | | | | | x | | x | | | x | |
| 381 | | x | | | | x | | x | | | | | | | | | x | | x | | | | x |
| 382 | | x | | | | x | | x | | | | | | | | | x | | | x | | x | |
| 383 | | x | | | | x | | x | | | | | | | | | x | | | x | | | x |
| 384 | | x | | | | x | | x | | | | | | | | | x | | | | x | x | |
| 385 | | x | | | | x | | x | | | | | | | | | x | | | | x | | x |
| 386 | | x | | | | x | | x | | | | | | | | | | x | x | | | x | |
| 387 | | x | | | | x | | x | | | | | | | | | | x | x | | | | x |
| 388 | | x | | | | x | | x | | | | | | | | | | x | | x | | x | |
| 389 | | x | | | | x | | x | | | | | | | | | | x | | x | | | x |
| 390 | | x | | | | x | | x | | | | | | | | | | x | | | x | x | |
| 391 | | x | | | | x | | x | | | | | | | | | | x | | | x | | x |
| 392 | | x | | | | x | | | x | | | | | x | | | | | x | | | x | |
| 393 | | x | | | | x | | | x | | | | | x | | | | | x | | | | x |
| 394 | | x | | | | x | | | x | | | | | x | | | | | | x | | x | |
| 395 | | x | | | | x | | | x | | | | | x | | | | | | x | | | x |
| 396 | | x | | | | x | | | x | | | | | x | | | | | | | x | x | |
| 397 | | x | | | | x | | | x | | | | | x | | | | | | | x | | x |
| 398 | | x | | | | x | | | x | | | | | | x | | | | x | | | x | |
| 399 | | x | | | | x | | | x | | | | | | x | | | | x | | | | x |
| 400 | | x | | | | x | | | x | | | | | | x | | | | | x | | x | |
| 401 | | x | | | | x | | | x | | | | | | x | | | | | x | | | x |
| 402 | | x | | | | x | | | x | | | | | | x | | | | | | x | x | |
| 403 | | x | | | | x | | | x | | | | | | x | | | | | | x | | x |
| 404 | | x | | | | x | | | x | | | | | | | x | | | x | | | x | |
| 405 | | x | | | | x | | | x | | | | | | | x | | | x | | | | x |
| 406 | | x | | | | x | | | x | | | | | | | x | | | | x | | x | |
| 407 | | x | | | | x | | | x | | | | | | | x | | | | x | | | x |
| 408 | | x | | | | x | | | x | | | | | | | x | | | | | x | x | |
| 409 | | x | | | | x | | | x | | | | | | | x | | | | | x | | x |
| 410 | | x | | | | x | | | x | | | | | | | | x | | x | | | x | |
| 411 | | x | | | | x | | | x | | | | | | | | x | | x | | | | x |
| 412 | | x | | | | x | | | x | | | | | | | | x | | | x | | x | |
| 413 | | x | | | | x | | | x | | | | | | | | x | | | x | | | x |
| 414 | | x | | | | x | | | x | | | | | | | | x | | | | x | x | |
| 415 | | x | | | | x | | | x | | | | | | | | x | | | | x | | x |
| 416 | | x | | | | x | | | x | | | | | | | | | x | x | | | x | |
| 417 | | x | | | | x | | | x | | | | | | | | | x | x | | | | x |
| 418 | | x | | | | x | | | x | | | | | | | | | x | | x | | x | |
| 419 | | x | | | | x | | | x | | | | | | | | | x | | x | | | x |
| 420 | | x | | | | x | | | x | | | | | | | | | x | | | x | x | |
| 421 | | x | | | | x | | | x | | | | | | | | | x | | | x | | x |
| 422 | | x | | | | x | | | | x | | | | x | | | | | x | | | x | |
| 423 | | x | | | | x | | | | x | | | | x | | | | | x | | | | x |
| 424 | | x | | | | x | | | | x | | | | x | | | | | | x | | x | |
| 425 | | x | | | | x | | | | x | | | | x | | | | | | x | | | x |
| 426 | | x | | | | x | | | | x | | | | x | | | | | | | x | x | |
| 427 | | x | | | | x | | | | x | | | | x | | | | | | | x | | x |
| 428 | | x | | | | x | | | | x | | | | | x | | | | x | | | x | |
| 429 | | x | | | | x | | | | x | | | | | x | | | | x | | | | x |
| 430 | | x | | | | x | | | | x | | | | | x | | | | | x | | x | |
| 431 | | x | | | | x | | | | x | | | | | x | | | | | x | | | x |
| 432 | | x | | | | x | | | | x | | | | | x | | | | | | x | x | |
| 433 | | x | | | | x | | | | x | | | | | x | | | | | | x | | x |
| 434 | | x | | | | x | | | | x | | | | | | x | | | x | | | x | |
| 435 | | x | | | | x | | | | x | | | | | | x | | | x | | | | x |
| 436 | | x | | | | x | | | | x | | | | | | x | | | | x | | x | |
| 437 | | x | | | | x | | | | x | | | | | | x | | | | x | | | x |
| 438 | | x | | | | x | | | | x | | | | | | x | | | | | x | x | |
| 439 | | x | | | | x | | | | x | | | | | | x | | | | | x | | x |
| 440 | | x | | | | x | | | | x | | | | | | | x | | x | | | x | |
| 441 | | x | | | | x | | | | x | | | | | | | x | | x | | | | x |
| 442 | | x | | | | x | | | | x | | | | | | | x | | | x | | x | |
| 443 | | x | | | | x | | | | x | | | | | | | x | | | x | | | x |
| 444 | | x | | | | x | | | | x | | | | | | | x | | | | x | x | |
| 445 | | x | | | | x | | | | x | | | | | | | x | | | | x | | x |
| 446 | | x | | | | x | | | | x | | | | | | | | x | x | | | x | |
| 447 | | x | | | | x | | | | x | | | | | | | | x | x | | | | x |
| 448 | | x | | | | x | | | | x | | | | | | | | x | | x | | x | |
| 449 | | x | | | | x | | | | x | | | | | | | | x | | x | | | x |
| 450 | | x | | | | x | | | | x | | | | | | | | x | | | x | x | |
| 451 | | x | | | | x | | | | x | | | | | | | | x | | | x | | x |
| 452 | | x | | | | x | | | | | x | | | x | | | | | x | | | x | |
| 453 | | x | | | | x | | | | | x | | | x | | | | | x | | | | x |
| 454 | | x | | | | x | | | | | x | | | x | | | | | | x | | x | |
| 455 | | x | | | | x | | | | | x | | | x | | | | | | x | | | x |
| 456 | | x | | | | x | | | | | x | | | x | | | | | | | x | x | |
| 457 | | x | | | | x | | | | | x | | | x | | | | | | | x | | x |
| 458 | | x | | | | x | | | | | x | | | | x | | | | x | | | x | |
| 459 | | x | | | | x | | | | | x | | | | x | | | | x | | | | x |
| 460 | | x | | | | x | | | | | x | | | | x | | | | | x | | x | |
| 461 | | x | | | | x | | | | | x | | | | x | | | | | x | | | x |
| 462 | | x | | | | x | | | | | x | | | | x | | | | | | x | x | |
| 463 | | x | | | | x | | | | | x | | | | x | | | | | | x | | x |
| 464 | | x | | | | x | | | | | x | | | | | x | | | x | | | x | |
| 465 | | x | | | | x | | | | | x | | | | | x | | | x | | | | x |
| 466 | | x | | | | x | | | | | x | | | | | x | | | | x | | x | |
| 467 | | x | | | | x | | | | | x | | | | | x | | | | x | | | x |
| 468 | | x | | | | x | | | | | x | | | | | x | | | | | x | x | |
| 469 | | x | | | | x | | | | | x | | | | | x | | | | | x | | x |
| 470 | | x | | | | x | | | | | x | | | | | | x | | x | | | x | |
| 471 | | x | | | | x | | | | | x | | | | | | x | | x | | | | x |
| 472 | | x | | | | x | | | | | x | | | | | | x | | | x | | x | |
| 473 | | x | | | | x | | | | | x | | | | | | x | | | x | | | x |
| 474 | | x | | | | x | | | | | x | | | | | | x | | | | x | x | |
| 475 | | x | | | | x | | | | | x | | | | | | x | | | | x | | x |
| 476 | | x | | | | x | | | | | x | | | | | | | x | x | | | x | |
| 477 | | x | | | | x | | | | | x | | | | | | | x | x | | | | x |
| 478 | | x | | | | x | | | | | x | | | | | | | x | | x | | x | |
| 479 | | x | | | | x | | | | | x | | | | | | | x | | x | | | x |
| 480 | | x | | | | x | | | | | x | | | | | | | x | | | x | x | |
| 481 | | x | | | | x | | | | | x | | | | | | | x | | | x | | x |
| 482 | | x | | | | x | | | | | | x | | x | | | | | x | | | x | |
| 483 | | x | | | | x | | | | | | x | | x | | | | | x | | | | x |
| 484 | | x | | | | x | | | | | | x | | x | | | | | | x | | x | |
| 485 | | x | | | | x | | | | | | x | | x | | | | | | x | | | x |
| 486 | | x | | | | x | | | | | | x | | x | | | | | | | x | x | |
| 487 | | x | | | | x | | | | | | x | | x | | | | | | | x | | x |
| 488 | | x | | | | x | | | | | | x | | | x | | | | x | | | x | |
| 489 | | x | | | | x | | | | | | x | | | x | | | | x | | | | x |
| 490 | | x | | | | x | | | | | | x | | | x | | | | | x | | x | |
| 491 | | x | | | | x | | | | | | x | | | x | | | | | x | | | x |
| 492 | | x | | | | x | | | | | | x | | | x | | | | | | x | x | |
| 493 | | x | | | | x | | | | | | x | | | x | | | | | | x | | x |
| 494 | | x | | | | x | | | | | | x | | | | x | | | x | | | x | |
| 495 | | x | | | | x | | | | | | x | | | | x | | | x | | | | x |
| 496 | | x | | | | x | | | | | | x | | | | x | | | | x | | x | |
| 497 | | x | | | | x | | | | | | x | | | | x | | | | x | | | x |
| 498 | | x | | | | x | | | | | | x | | | | x | | | | | x | x | |
| 499 | | x | | | | x | | | | | | x | | | | x | | | | | x | | x |
| 500 | | x | | | | x | | | | | | x | | | | | x | | x | | | x | |
| 501 | | x | | | | x | | | | | | x | | | | | x | | x | | | | x |
| 502 | | x | | | | x | | | | | | x | | | | | x | | | x | | x | |
| 503 | | x | | | | x | | | | | | x | | | | | x | | | x | | | x |
| 504 | | x | | | | x | | | | | | x | | | | | x | | | | x | x | |
| 505 | | x | | | | x | | | | | | x | | | | | x | | | | x | | x |
| 506 | | x | | | | x | | | | | | x | | | | | | x | x | | | x | |
| 507 | | x | | | | x | | | | | | x | | | | | | x | x | | | | x |
| 508 | | x | | | | x | | | | | | x | | | | | | x | | x | | x | |
| 509 | | x | | | | x | | | | | | x | | | | | | x | | x | | | x |
| 510 | | x | | | | x | | | | | | x | | | | | | x | | | x | x | |
| 511 | | x | | | | x | | | | | | x | | | | | | x | | | x | | x |
| 512 | | x | | | | x | | | | | | | x | x | | | | | x | | | x | |
| 513 | | x | | | | x | | | | | | | x | x | | | | | x | | | | x |
| 514 | | x | | | | x | | | | | | | x | x | | | | | | x | | x | |
| 515 | | x | | | | x | | | | | | | x | x | | | | | | x | | | x |
| 516 | | x | | | | x | | | | | | | x | x | | | | | | | x | x | |
| 517 | | x | | | | x | | | | | | | x | x | | | | | | | x | | x |
| 518 | | x | | | | x | | | | | | | x | | x | | | | x | | | x | |
| 519 | | x | | | | x | | | | | | | x | | x | | | | x | | | | x |
| 520 | | x | | | | x | | | | | | | x | | x | | | | | x | | x | |
| 521 | | x | | | | x | | | | | | | x | | x | | | | | x | | | x |
| 522 | | x | | | | x | | | | | | | x | | x | | | | | | x | x | |
| 523 | | x | | | | x | | | | | | | x | | x | | | | | | x | | x |
| 524 | | x | | | | x | | | | | | | x | | | x | | | x | | | x | |
| 525 | | x | | | | x | | | | | | | x | | | x | | | x | | | | x |
| 526 | | x | | | | x | | | | | | | x | | | x | | | | x | | x | |
| 527 | | x | | | | x | | | | | | | x | | | x | | | | x | | | x |
| 528 | | x | | | | x | | | | | | | x | | | x | | | | | x | x | |
| 529 | | x | | | | x | | | | | | | x | | | x | | | | | x | | x |
| 530 | | x | | | | x | | | | | | | x | | | | x | | x | | | x | |
| 531 | | x | | | | x | | | | | | | x | | | | x | | x | | | | x |
| 532 | | x | | | | x | | | | | | | x | | | | x | | | x | | x | |
| 533 | | x | | | | x | | | | | | | x | | | | x | | | x | | | x |
| 534 | | x | | | | x | | | | | | | x | | | | x | | | | x | x | |
| 535 | | x | | | | x | | | | | | | x | | | | x | | | | x | | x |
| 536 | | x | | | | x | | | | | | | x | | | | | x | x | | | x | |
| 537 | | x | | | | x | | | | | | | x | | | | | x | x | | | | x |
| 538 | | x | | | | x | | | | | | | x | | | | | x | | x | | x | |
| 539 | | x | | | | x | | | | | | | x | | | | | x | | x | | | x |
| 540 | | x | | | | x | | | | | | | x | | | | | x | | | x | x | |
| 541 | | x | | | | x | | | | | | | x | | | | | x | | | x | | x |
| 542 | | x | | | | | x | x | | | | | | x | | | | | x | | | x | |
| 543 | | x | | | | | x | x | | | | | | x | | | | | x | | | | x |
| 544 | | x | | | | | x | x | | | | | | x | | | | | | x | | x | |
| 545 | | x | | | | | x | x | | | | | | x | | | | | | x | | | x |
| 546 | | x | | | | | x | x | | | | | | x | | | | | | | x | x | |
| 547 | | x | | | | | x | x | | | | | | x | | | | | | | x | | x |
| 548 | | x | | | | | x | x | | | | | | | x | | | | x | | | x | |
| 549 | | x | | | | | x | x | | | | | | | x | | | | x | | | | x |
| 550 | | x | | | | | x | x | | | | | | | x | | | | | x | | x | |
| 551 | | x | | | | | x | x | | | | | | | x | | | | | x | | | x |
| 552 | | x | | | | | x | x | | | | | | | x | | | | | | x | x | |
| 553 | | x | | | | | x | x | | | | | | | x | | | | | | x | | x |
| 554 | | x | | | | | x | x | | | | | | | | x | | | x | | | x | |
| 555 | | x | | | | | x | x | | | | | | | | x | | | x | | | | x |
| 556 | | x | | | | | x | x | | | | | | | | x | | | | x | | x | |
| 557 | | x | | | | | x | x | | | | | | | | x | | | | x | | | x |
| 558 | | x | | | | | x | x | | | | | | | | x | | | | | x | x | |
| 559 | | x | | | | | x | x | | | | | | | | x | | | | | x | | x |
| 560 | | x | | | | | x | x | | | | | | | | | x | | x | | | x | |
| 561 | | x | | | | | x | x | | | | | | | | | x | | x | | | | x |
| 562 | | x | | | | | x | x | | | | | | | | | x | | | x | | x | |
| 563 | | x | | | | | x | x | | | | | | | | | x | | | x | | | x |
| 564 | | x | | | | | x | x | | | | | | | | | x | | | | x | x | |
| 565 | | x | | | | | x | x | | | | | | | | | x | | | | x | | x |
| 566 | | x | | | | | x | x | | | | | | | | | | x | x | | | x | |
| 567 | | x | | | | | x | x | | | | | | | | | | x | x | | | | x |
| 568 | | x | | | | | x | x | | | | | | | | | | x | | x | | x | |
| 569 | | x | | | | | x | x | | | | | | | | | | x | | x | | | x |
| 570 | | x | | | | | x | x | | | | | | | | | | x | | | x | x | |
| 571 | | x | | | | | x | x | | | | | | | | | | x | | | x | | x |
| 572 | | x | | | | | x | | x | | | | | x | | | | | x | | | x | |
| 573 | | x | | | | | x | | x | | | | | x | | | | | x | | | | x |
| 574 | | x | | | | | x | | x | | | | | x | | | | | | x | | x | |
| 575 | | x | | | | | x | | x | | | | | x | | | | | | x | | | x |
| 576 | | x | | | | | x | | x | | | | | x | | | | | | | x | x | |
| 577 | | x | | | | | x | | x | | | | | x | | | | | | | x | | x |
| 578 | | x | | | | | x | | x | | | | | | x | | | | x | | | x | |
| 579 | | x | | | | | x | | x | | | | | | x | | | | x | | | | x |
| 580 | | x | | | | | x | | x | | | | | | x | | | | | x | | x | |
| 581 | | x | | | | | x | | x | | | | | | x | | | | | x | | | x |
| 582 | | x | | | | | x | | x | | | | | | x | | | | | | x | x | |
| 583 | | x | | | | | x | | x | | | | | | x | | | | | | x | | x |
| 584 | | x | | | | | x | | x | | | | | | | x | | | x | | | x | |
| 585 | | x | | | | | x | | x | | | | | | | x | | | x | | | | x |
| 586 | | x | | | | | x | | x | | | | | | | x | | | | x | | x | |
| 587 | | x | | | | | x | | x | | | | | | | x | | | | x | | | x |
| 588 | | x | | | | | x | | x | | | | | | | x | | | | | x | x | |
| 589 | | x | | | | | x | | x | | | | | | | x | | | | | x | | x |
| 590 | | x | | | | | x | | x | | | | | | | | x | | x | | | x | |
| 591 | | x | | | | | x | | x | | | | | | | | x | | x | | | | x |
| 592 | | x | | | | | x | | x | | | | | | | | x | | | x | | x | |
| 593 | | x | | | | | x | | x | | | | | | | | x | | | x | | | x |
| 594 | | x | | | | | x | | x | | | | | | | | x | | | | x | x | |
| 595 | | x | | | | | x | | x | | | | | | | | x | | | | x | | x |
| 596 | | x | | | | | x | | x | | | | | | | | | x | x | | | x | |
| 597 | | x | | | | | x | | x | | | | | | | | | x | x | | | | x |
| 598 | | x | | | | | x | | x | | | | | | | | | x | | x | | x | |
| 599 | | x | | | | | x | | x | | | | | | | | | x | | x | | | x |
| 600 | | x | | | | | x | | x | | | | | | | | | x | | | x | x | |
| 601 | | x | | | | | x | | x | | | | | | | | | x | | | x | | x |
| 602 | | x | | | | | x | | | x | | | | x | | | | | x | | | x | |
| 603 | | x | | | | | x | | | x | | | | x | | | | | x | | | | x |
| 604 | | x | | | | | x | | | x | | | | x | | | | | | x | | x | |
| 605 | | x | | | | | x | | | x | | | | x | | | | | | x | | | x |
| 606 | | x | | | | | x | | | x | | | | x | | | | | | | x | x | |
| 607 | | x | | | | | x | | | x | | | | x | | | | | | | x | | x |
| 608 | | x | | | | | x | | | x | | | | | x | | | | x | | | x | |
| 609 | | x | | | | | x | | | x | | | | | x | | | | x | | | | x |
| 610 | | x | | | | | x | | | x | | | | | x | | | | | x | | x | |
| 611 | | x | | | | | x | | | x | | | | | x | | | | | x | | | x |
| 612 | | x | | | | | x | | | x | | | | | x | | | | | | x | x | |
| 613 | | x | | | | | x | | | x | | | | | x | | | | | | x | | x |
| 614 | | x | | | | | x | | | x | | | | | | x | | | x | | | x | |
| 615 | | x | | | | | x | | | x | | | | | | x | | | x | | | | x |
| 616 | | x | | | | | x | | | x | | | | | | x | | | | x | | x | |
| 617 | | x | | | | | x | | | x | | | | | | x | | | | x | | | x |
| 618 | | x | | | | | x | | | x | | | | | | x | | | | | x | x | |
| 619 | | x | | | | | x | | | x | | | | | | x | | | | | x | | x |
| 620 | | x | | | | | x | | | x | | | | | | | x | | x | | | x | |
| 621 | | x | | | | | x | | | x | | | | | | | x | | x | | | | x |
| 622 | | x | | | | | x | | | x | | | | | | | x | | | x | | x | |
| 623 | | x | | | | | x | | | x | | | | | | | x | | | x | | | x |
| 624 | | x | | | | | x | | | x | | | | | | | x | | | | x | x | |
| 625 | | x | | | | | x | | | x | | | | | | | x | | | | x | | x |
| 626 | | x | | | | | x | | | x | | | | | | | | x | x | | | x | |
| 627 | | x | | | | | x | | | x | | | | | | | | x | x | | | | x |
| 628 | | x | | | | | x | | | x | | | | | | | | x | | x | | x | |
| 629 | | x | | | | | x | | | x | | | | | | | | x | | x | | | x |
| 630 | | x | | | | | x | | | x | | | | | | | | x | | | x | x | |
| 631 | | x | | | | | x | | | x | | | | | | | | x | | | x | | x |
| 632 | | x | | | | | x | | | | x | | | x | | | | | x | | | x | |
| 633 | | x | | | | | x | | | | x | | | x | | | | | x | | | | x |
| 634 | | x | | | | | x | | | | x | | | x | | | | | | x | | x | |
| 635 | | x | | | | | x | | | | x | | | x | | | | | | x | | | x |
| 636 | | x | | | | | x | | | | x | | | x | | | | | | | x | x | |
| 637 | | x | | | | | x | | | | x | | | x | | | | | | | x | | x |
| 638 | | x | | | | | x | | | | x | | | | x | | | | x | | | x | |
| 639 | | x | | | | | x | | | | x | | | | x | | | | x | | | | x |
| 640 | | x | | | | | x | | | | x | | | | x | | | | | x | | x | |
| 641 | | x | | | | | x | | | | x | | | | x | | | | | x | | | x |
| 642 | | x | | | | | x | | | | x | | | | x | | | | | | x | x | |
| 643 | | x | | | | | x | | | | x | | | | x | | | | | | x | | x |
| 644 | | x | | | | | x | | | | x | | | | | x | | | x | | | x | |
| 645 | | x | | | | | x | | | | x | | | | | x | | | x | | | | x |
| 646 | | x | | | | | x | | | | x | | | | | x | | | | x | | x | |
| 647 | | x | | | | | x | | | | x | | | | | x | | | | x | | | x |
| 648 | | x | | | | | x | | | | x | | | | | x | | | | | x | x | |
| 649 | | x | | | | | x | | | | x | | | | | x | | | | | x | | x |
| 650 | | x | | | | | x | | | | x | | | | | | x | | x | | | x | |
| 651 | | x | | | | | x | | | | x | | | | | | x | | x | | | | x |
| 652 | | x | | | | | x | | | | x | | | | | | x | | | x | | x | |
| 653 | | x | | | | | x | | | | x | | | | | | x | | | x | | | x |
| 654 | | x | | | | | x | | | | x | | | | | | x | | | | x | x | |
| 655 | | x | | | | | x | | | | x | | | | | | x | | | | x | | x |
| 656 | | x | | | | | x | | | | x | | | | | | | x | x | | | x | |
| 657 | | x | | | | | x | | | | x | | | | | | | x | x | | | | x |
| 658 | | x | | | | | x | | | | x | | | | | | | x | | x | | x | |
| 659 | | x | | | | | x | | | | x | | | | | | | x | | x | | | x |
| 660 | | x | | | | | x | | | | x | | | | | | | x | | | x | x | |
| 661 | | x | | | | | x | | | | x | | | | | | | x | | | x | | x |
| 662 | | x | | | | | x | | | | | x | | x | | | | | x | | | x | |
| 663 | | x | | | | | x | | | | | x | | x | | | | | x | | | | x |
| 664 | | x | | | | | x | | | | | x | | x | | | | | | x | | x | |
| 665 | | x | | | | | x | | | | | x | | x | | | | | | x | | | x |
| 666 | | x | | | | | x | | | | | x | | x | | | | | | | x | x | |
| 667 | | x | | | | | x | | | | | x | | x | | | | | | | x | | x |
| 668 | | x | | | | | x | | | | | x | | | x | | | | x | | | x | |
| 669 | | x | | | | | x | | | | | x | | | x | | | | x | | | | x |
| 670 | | x | | | | | x | | | | | x | | | x | | | | | x | | x | |
| 671 | | x | | | | | x | | | | | x | | | x | | | | | x | | | x |
| 672 | | x | | | | | x | | | | | x | | | x | | | | | | x | x | |
| 673 | | x | | | | | x | | | | | x | | | x | | | | | | x | | x |
| 674 | | x | | | | | x | | | | | x | | | | x | | | x | | | x | |
| 675 | | x | | | | | x | | | | | x | | | | x | | | x | | | | x |
| 676 | | x | | | | | x | | | | | x | | | | x | | | | x | | x | |
| 677 | | x | | | | | x | | | | | x | | | | x | | | | x | | | x |
| 678 | | x | | | | | x | | | | | x | | | | x | | | | | x | x | |
| 679 | | x | | | | | x | | | | | x | | | | x | | | | | x | | x |
| 680 | | x | | | | | x | | | | | x | | | | | x | | x | | | x | |
| 681 | | x | | | | | x | | | | | x | | | | | x | | x | | | | x |
| 682 | | x | | | | | x | | | | | x | | | | | x | | | x | | x | |
| 683 | | x | | | | | x | | | | | x | | | | | x | | | x | | | x |
| 684 | | x | | | | | x | | | | | x | | | | | x | | | | x | x | |
| 685 | | x | | | | | x | | | | | x | | | | | x | | | | x | | x |
| 686 | | x | | | | | x | | | | | x | | | | | | x | x | | | x | |
| 687 | | x | | | | | x | | | | | x | | | | | | x | x | | | | x |
| 688 | | x | | | | | x | | | | | x | | | | | | x | | x | | x | |
| 689 | | x | | | | | x | | | | | x | | | | | | x | | x | | | x |
| 690 | | x | | | | | x | | | | | x | | | | | | x | | | x | x | |
| 691 | | x | | | | | x | | | | | x | | | | | | x | | | x | | x |
| 692 | | x | | | | | x | | | | | | x | x | | | | | x | | | x | |
| 693 | | x | | | | | x | | | | | | x | x | | | | | x | | | | x |
| 694 | | x | | | | | x | | | | | | x | x | | | | | | x | | x | |
| 695 | | x | | | | | x | | | | | | x | x | | | | | | x | | | x |
| 696 | | x | | | | | x | | | | | | x | x | | | | | | | x | x | |
| 697 | | x | | | | | x | | | | | | x | x | | | | | | | x | | x |
| 698 | | x | | | | | x | | | | | | x | | x | | | | x | | | x | |
| 699 | | x | | | | | x | | | | | | x | | x | | | | x | | | | x |
| 700 | | x | | | | | x | | | | | | x | | x | | | | | x | | x | |
| 701 | | x | | | | | x | | | | | | x | | x | | | | | x | | | x |
| 702 | | x | | | | | x | | | | | | x | | x | | | | | | x | x | |
| 703 | | x | | | | | x | | | | | | x | | x | | | | | | x | | x |
| 704 | | x | | | | | x | | | | | | x | | | x | | | x | | | x | |
| 705 | | x | | | | | x | | | | | | x | | | x | | | x | | | | x |
| 706 | | x | | | | | x | | | | | | x | | | x | | | | x | | x | |
| 707 | | x | | | | | x | | | | | | x | | | x | | | | x | | | x |
| 708 | | x | | | | | x | | | | | | x | | | x | | | | | x | x | |
| 709 | | x | | | | | x | | | | | | x | | | x | | | | | x | | x |
| 710 | | x | | | | | x | | | | | | x | | | | x | | x | | | x | |
| 711 | | x | | | | | x | | | | | | x | | | | x | | x | | | | x |
| 712 | | x | | | | | x | | | | | | x | | | | x | | | x | | x | |
| 713 | | x | | | | | x | | | | | | x | | | | x | | | x | | | x |
| 714 | | x | | | | | x | | | | | | x | | | | x | | | | x | x | |
| 715 | | x | | | | | x | | | | | | x | | | | x | | | | x | | x |
| 716 | | x | | | | | x | | | | | | x | | | | | x | x | | | x | |
| 717 | | x | | | | | x | | | | | | x | | | | | x | x | | | | x |
| 718 | | x | | | | | x | | | | | | x | | | | | x | | x | | x | |
| 719 | | x | | | | | x | | | | | | x | | | | | x | | x | | | x |
| 720 | | x | | | | | x | | | | | | x | | | | | x | | | x | x | |
| 721 | | x | | | | | x | | | | | | x | | | | | x | | | x | | x |
| 722 | | | x | | | x | | x | | | | | | x | | | | | x | | | x | |
| 723 | | | x | | | x | | x | | | | | | x | | | | | x | | | | x |
| 724 | | | x | | | x | | x | | | | | | x | | | | | | x | | x | |
| 725 | | | x | | | x | | x | | | | | | x | | | | | | x | | | x |
| 726 | | | x | | | x | | x | | | | | | x | | | | | | | x | x | |
| 727 | | | x | | | x | | x | | | | | | x | | | | | | | x | | x |
| 728 | | | x | | | x | | x | | | | | | | x | | | | x | | | x | |
| 729 | | | x | | | x | | x | | | | | | | x | | | | x | | | | x |
| 730 | | | x | | | x | | x | | | | | | | x | | | | | x | | x | |
| 731 | | | x | | | x | | x | | | | | | | x | | | | | x | | | x |
| 732 | | | x | | | x | | x | | | | | | | x | | | | | | x | x | |
| 733 | | | x | | | x | | x | | | | | | | x | | | | | | x | | x |
| 734 | | | x | | | x | | x | | | | | | | | x | | | x | | | x | |
| 735 | | | x | | | x | | x | | | | | | | | x | | | x | | | | x |
| 736 | | | x | | | x | | x | | | | | | | | x | | | | x | | x | |
| 737 | | | x | | | x | | x | | | | | | | | x | | | | x | | | x |
| 738 | | | x | | | x | | x | | | | | | | | x | | | | | x | x | |
| 739 | | | x | | | x | | x | | | | | | | | x | | | | | x | | x |
| 740 | | | x | | | x | | x | | | | | | | | | x | | x | | | x | |
| 741 | | | x | | | x | | x | | | | | | | | | x | | x | | | | x |
| 742 | | | x | | | x | | x | | | | | | | | | x | | | x | | x | |
| 743 | | | x | | | x | | x | | | | | | | | | x | | | x | | | x |
| 744 | | | x | | | x | | x | | | | | | | | | x | | | | x | x | |
| 745 | | | x | | | x | | x | | | | | | | | | x | | | | x | | x |
| 746 | | | x | | | x | | x | | | | | | | | | | x | x | | | x | |
| 747 | | | x | | | x | | x | | | | | | | | | | x | x | | | | x |
| 748 | | | x | | | x | | x | | | | | | | | | | x | | x | | x | |
| 749 | | | x | | | x | | x | | | | | | | | | | x | | x | | | x |
| 750 | | | x | | | x | | x | | | | | | | | | | x | | | x | x | |
| 751 | | | x | | | x | | x | | | | | | | | | | x | | | x | | x |
| 752 | | | x | | | x | | | x | | | | | x | | | | | x | | | x | |
| 753 | | | x | | | x | | | x | | | | | x | | | | | x | | | | x |
| 754 | | | x | | | x | | | x | | | | | x | | | | | | x | | x | |
| 755 | | | x | | | x | | | x | | | | | x | | | | | | x | | | x |
| 756 | | | x | | | x | | | x | | | | | x | | | | | | | x | x | |
| 757 | | | x | | | x | | | x | | | | | x | | | | | | | x | | x |
| 758 | | | x | | | x | | | x | | | | | | x | | | | x | | | x | |
| 759 | | | x | | | x | | | x | | | | | | x | | | | x | | | | x |
| 760 | | | x | | | x | | | x | | | | | | x | | | | | x | | x | |
| 761 | | | x | | | x | | | x | | | | | | x | | | | | x | | | x |
| 762 | | | x | | | x | | | x | | | | | | x | | | | | | x | x | |
| 763 | | | x | | | x | | | x | | | | | | x | | | | | | x | | x |
| 764 | | | x | | | x | | | x | | | | | | | x | | | x | | | x | |
| 765 | | | x | | | x | | | x | | | | | | | x | | | x | | | | x |
| 766 | | | x | | | x | | | x | | | | | | | x | | | | x | | x | |
| 767 | | | x | | | x | | | x | | | | | | | x | | | | x | | | x |
| 768 | | | x | | | x | | | x | | | | | | | x | | | | | x | x | |
| 769 | | | x | | | x | | | x | | | | | | | x | | | | | x | | x |
| 770 | | | x | | | x | | | x | | | | | | | | x | | x | | | x | |
| 771 | | | x | | | x | | | x | | | | | | | | x | | x | | | | x |
| 772 | | | x | | | x | | | x | | | | | | | | x | | | x | | x | |
| 773 | | | x | | | x | | | x | | | | | | | | x | | | x | | | x |
| 774 | | | x | | | x | | | x | | | | | | | | x | | | | x | x | |
| 775 | | | x | | | x | | | x | | | | | | | | x | | | | x | | x |
| 776 | | | x | | | x | | | x | | | | | | | | | x | x | | | x | |
| 777 | | | x | | | x | | | x | | | | | | | | | x | x | | | | x |
| 778 | | | x | | | x | | | x | | | | | | | | | x | | x | | x | |
| 779 | | | x | | | x | | | x | | | | | | | | | x | | x | | | x |
| 780 | | | x | | | x | | | x | | | | | | | | | x | | | x | x | |
| 781 | | | x | | | x | | | x | | | | | | | | | x | | | x | | x |
| 782 | | | x | | | x | | | | x | | | | x | | | | | x | | | x | |
| 783 | | | x | | | x | | | | x | | | | x | | | | | x | | | | x |
| 784 | | | x | | | x | | | | x | | | | x | | | | | | x | | x | |
| 785 | | | x | | | x | | | | x | | | | x | | | | | | x | | | x |
| 786 | | | x | | | x | | | | x | | | | x | | | | | | | x | x | |
| 787 | | | x | | | x | | | | x | | | | x | | | | | | | x | | x |
| 788 | | | x | | | x | | | | x | | | | | x | | | | x | | | x | |
| 789 | | | x | | | x | | | | x | | | | | x | | | | x | | | | x |
| 790 | | | x | | | x | | | | x | | | | | x | | | | | x | | x | |
| 791 | | | x | | | x | | | | x | | | | | x | | | | | x | | | x |
| 792 | | | x | | | x | | | | x | | | | | x | | | | | | x | x | |
| 793 | | | x | | | x | | | | x | | | | | x | | | | | | x | | x |
| 794 | | | x | | | x | | | | x | | | | | | x | | | x | | | x | |
| 795 | | | x | | | x | | | | x | | | | | | x | | | x | | | | x |
| 796 | | | x | | | x | | | | x | | | | | | x | | | | x | | x | |
| 797 | | | x | | | x | | | | x | | | | | | x | | | | x | | | x |
| 798 | | | x | | | x | | | | x | | | | | | x | | | | | x | x | |
| 799 | | | x | | | x | | | | x | | | | | | x | | | | | x | | x |
| 800 | | | x | | | x | | | | x | | | | | | | x | | x | | | x | |
| 801 | | | x | | | x | | | | x | | | | | | | x | | x | | | | x |
| 802 | | | x | | | x | | | | x | | | | | | | x | | | x | | x | |
| 803 | | | x | | | x | | | | x | | | | | | | x | | | x | | | x |
| 804 | | | x | | | x | | | | x | | | | | | | x | | | | x | x | |
| 805 | | | x | | | x | | | | x | | | | | | | x | | | | x | | x |
| 806 | | | x | | | x | | | | x | | | | | | | | x | x | | | x | |
| 807 | | | x | | | x | | | | x | | | | | | | | x | x | | | | x |
| 808 | | | x | | | x | | | | x | | | | | | | | x | | x | | x | |
| 809 | | | x | | | x | | | | x | | | | | | | | x | | x | | | x |
| 810 | | | x | | | x | | | | x | | | | | | | | x | | | x | x | |
| 811 | | | x | | | x | | | | x | | | | | | | | x | | | x | | x |
| 812 | | | x | | | x | | | | | x | | | x | | | | | x | | | x | |
| 813 | | | x | | | x | | | | | x | | | x | | | | | x | | | | x |
| 814 | | | x | | | x | | | | | x | | | x | | | | | | x | | x | |
| 815 | | | x | | | x | | | | | x | | | x | | | | | | x | | | x |
| 816 | | | x | | | x | | | | | x | | | x | | | | | | | x | x | |
| 817 | | | x | | | x | | | | | x | | | x | | | | | | | x | | x |
| 818 | | | x | | | x | | | | | x | | | | x | | | | x | | | x | |
| 819 | | | x | | | x | | | | | x | | | | x | | | | x | | | | x |
| 820 | | | x | | | x | | | | | x | | | | x | | | | | x | | x | |
| 821 | | | x | | | x | | | | | x | | | | x | | | | | x | | | x |
| 822 | | | x | | | x | | | | | x | | | | x | | | | | | x | x | |
| 823 | | | x | | | x | | | | | x | | | | x | | | | | | x | | x |
| 824 | | | x | | | x | | | | | x | | | | | x | | | x | | | x | |
| 825 | | | x | | | x | | | | | x | | | | | x | | | x | | | | x |
| 826 | | | x | | | x | | | | | x | | | | | x | | | | x | | x | |
| 827 | | | x | | | x | | | | | x | | | | | x | | | | x | | | x |
| 828 | | | x | | | x | | | | | x | | | | | x | | | | | x | x | |
| 829 | | | x | | | x | | | | | x | | | | | x | | | | | x | | x |
| 830 | | | x | | | x | | | | | x | | | | | | x | | x | | | x | |
| 831 | | | x | | | x | | | | | x | | | | | | x | | x | | | | x |
| 832 | | | x | | | x | | | | | x | | | | | | x | | | x | | x | |
| 833 | | | x | | | x | | | | | x | | | | | | x | | | x | | | x |
| 834 | | | x | | | x | | | | | x | | | | | | x | | | | x | x | |
| 835 | | | x | | | x | | | | | x | | | | | | x | | | | x | | x |
| 836 | | | x | | | x | | | | | x | | | | | | | x | x | | | x | |
| 837 | | | x | | | x | | | | | x | | | | | | | x | x | | | | x |
| 838 | | | x | | | x | | | | | x | | | | | | | x | | x | | x | |
| 839 | | | x | | | x | | | | | x | | | | | | | x | | x | | | x |
| 840 | | | x | | | x | | | | | x | | | | | | | x | | | x | x | |
| 841 | | | x | | | x | | | | | x | | | | | | | x | | | x | | x |
| 842 | | | x | | | x | | | | | | x | | x | | | | | x | | | x | |
| 843 | | | x | | | x | | | | | | x | | x | | | | | x | | | | x |
| 844 | | | x | | | x | | | | | | x | | x | | | | | | x | | x | |
| 845 | | | x | | | x | | | | | | x | | x | | | | | | x | | | x |
| 846 | | | x | | | x | | | | | | x | | x | | | | | | | x | x | |
| 847 | | | x | | | x | | | | | | x | | x | | | | | | | x | | x |
| 848 | | | x | | | x | | | | | | x | | | x | | | | x | | | x | |
| 849 | | | x | | | x | | | | | | x | | | x | | | | x | | | | x |
| 850 | | | x | | | x | | | | | | x | | | x | | | | | x | | x | |
| 851 | | | x | | | x | | | | | | x | | | x | | | | | x | | | x |
| 852 | | | x | | | x | | | | | | x | | | x | | | | | | x | x | |
| 853 | | | x | | | x | | | | | | x | | | x | | | | | | x | | x |
| 854 | | | x | | | x | | | | | | x | | | | x | | | x | | | x | |
| 855 | | | x | | | x | | | | | | x | | | | x | | | x | | | | x |
| 856 | | | x | | | x | | | | | | x | | | | x | | | | x | | x | |
| 857 | | | x | | | x | | | | | | x | | | | x | | | | x | | | x |
| 858 | | | x | | | x | | | | | | x | | | | x | | | | | x | x | |
| 859 | | | x | | | x | | | | | | x | | | | x | | | | | x | | x |
| 860 | | | x | | | x | | | | | | x | | | | | x | | x | | | x | |
| 861 | | | x | | | x | | | | | | x | | | | | x | | x | | | | x |
| 862 | | | x | | | x | | | | | | x | | | | | x | | | x | | x | |
| 863 | | | x | | | x | | | | | | x | | | | | x | | | x | | | x |
| 864 | | | x | | | x | | | | | | x | | | | | x | | | | x | x | |
| 865 | | | x | | | x | | | | | | x | | | | | x | | | | x | | x |
| 866 | | | x | | | x | | | | | | x | | | | | | x | x | | | x | |
| 867 | | | x | | | x | | | | | | x | | | | | | x | x | | | | x |
| 868 | | | x | | | x | | | | | | x | | | | | | x | | x | | x | |
| 869 | | | x | | | x | | | | | | x | | | | | | x | | x | | | x |
| 870 | | | x | | | x | | | | | | x | | | | | | x | | | x | x | |
| 871 | | | x | | | x | | | | | | x | | | | | | x | | | x | | x |
| 872 | | | x | | | x | | | | | | | x | x | | | | | x | | | x | |
| 873 | | | x | | | x | | | | | | | x | x | | | | | x | | | | x |
| 874 | | | x | | | x | | | | | | | x | x | | | | | | x | | x | |
| 875 | | | x | | | x | | | | | | | x | x | | | | | | x | | | x |
| 876 | | | x | | | x | | | | | | | x | x | | | | | | | x | x | |
| 877 | | | x | | | x | | | | | | | x | x | | | | | | | x | | x |
| 878 | | | x | | | x | | | | | | | x | | x | | | | x | | | x | |
| 879 | | | x | | | x | | | | | | | x | | x | | | | x | | | | x |
| 880 | | | x | | | x | | | | | | | x | | x | | | | | x | | x | |
| 881 | | | x | | | x | | | | | | | x | | x | | | | | x | | | x |
| 882 | | | x | | | x | | | | | | | x | | x | | | | | | x | x | |
| 883 | | | x | | | x | | | | | | | x | | x | | | | | | x | | x |
| 884 | | | x | | | x | | | | | | | x | | | x | | | x | | | x | |
| 885 | | | x | | | x | | | | | | | x | | | x | | | x | | | | x |
| 886 | | | x | | | x | | | | | | | x | | | x | | | | x | | x | |
| 887 | | | x | | | x | | | | | | | x | | | x | | | | x | | | x |
| 888 | | | x | | | x | | | | | | | x | | | x | | | | | x | x | |
| 889 | | | x | | | x | | | | | | | x | | | x | | | | | x | | x |
| 890 | | | x | | | x | | | | | | | x | | | | x | | x | | | x | |
| 891 | | | x | | | x | | | | | | | x | | | | x | | x | | | | x |
| 892 | | | x | | | x | | | | | | | x | | | | x | | | x | | x | |
| 893 | | | x | | | x | | | | | | | x | | | | x | | | x | | | x |
| 894 | | | x | | | x | | | | | | | x | | | | x | | | | x | x | |
| 895 | | | x | | | x | | | | | | | x | | | | x | | | | x | | x |
| 896 | | | x | | | x | | | | | | | x | | | | | x | x | | | x | |
| 897 | | | x | | | x | | | | | | | x | | | | | x | x | | | | x |
| 898 | | | x | | | x | | | | | | | x | | | | | x | | x | | x | |
| 899 | | | x | | | x | | | | | | | x | | | | | x | | x | | | x |
| 900 | | | x | | | x | | | | | | | x | | | | | x | | | x | x | |
| 901 | | | x | | | x | | | | | | | x | | | | | x | | | x | | x |
| 902 | | | x | | | | x | x | | | | | | x | | | | | x | | | x | |
| 903 | | | x | | | | x | x | | | | | | x | | | | | x | | | | x |
| 904 | | | x | | | | x | x | | | | | | x | | | | | | x | | x | |
| 905 | | | x | | | | x | x | | | | | | x | | | | | | x | | | x |
| 906 | | | x | | | | x | x | | | | | | x | | | | | | | x | x | |
| 907 | | | x | | | | x | x | | | | | | x | | | | | | | x | | x |
| 908 | | | x | | | | x | x | | | | | | | x | | | | x | | | x | |
| 909 | | | x | | | | x | x | | | | | | | x | | | | x | | | | x |
| 910 | | | x | | | | x | x | | | | | | | x | | | | | x | | x | |
| 911 | | | x | | | | x | x | | | | | | | x | | | | | x | | | x |
| 912 | | | x | | | | x | x | | | | | | | x | | | | | | x | x | |
| 913 | | | x | | | | x | x | | | | | | | x | | | | | | x | | x |
| 914 | | | x | | | | x | x | | | | | | | | x | | | x | | | x | |
| 915 | | | x | | | | x | x | | | | | | | | x | | | x | | | | x |
| 916 | | | x | | | | x | x | | | | | | | | x | | | | x | | x | |
| 917 | | | x | | | | x | x | | | | | | | | x | | | | x | | | x |
| 918 | | | x | | | | x | x | | | | | | | | x | | | | | x | x | |
| 919 | | | x | | | | x | x | | | | | | | | x | | | | | x | | x |
| 920 | | | x | | | | x | x | | | | | | | | | x | | x | | | x | |
| 921 | | | x | | | | x | x | | | | | | | | | x | | x | | | | x |
| 922 | | | x | | | | x | x | | | | | | | | | x | | | x | | x | |
| 923 | | | x | | | | x | x | | | | | | | | | x | | | x | | | x |
| 924 | | | x | | | | x | x | | | | | | | | | x | | | | x | x | |
| 925 | | | x | | | | x | x | | | | | | | | | x | | | | x | | x |
| 926 | | | x | | | | x | x | | | | | | | | | | x | x | | | x | |
| 927 | | | x | | | | x | x | | | | | | | | | | x | x | | | | x |
| 928 | | | x | | | | x | x | | | | | | | | | | x | | x | | x | |
| 929 | | | x | | | | x | x | | | | | | | | | | x | | x | | | x |
| 930 | | | x | | | | x | x | | | | | | | | | | x | | | x | x | |
| 931 | | | x | | | | x | x | | | | | | | | | | x | | | x | | x |
| 932 | | | x | | | | x | | x | | | | | x | | | | | x | | | x | |
| 933 | | | x | | | | x | | x | | | | | x | | | | | x | | | | x |
| 934 | | | x | | | | x | | x | | | | | x | | | | | | x | | x | |
| 935 | | | x | | | | x | | x | | | | | x | | | | | | x | | | x |
| 936 | | | x | | | | x | | x | | | | | x | | | | | | | x | x | |
| 937 | | | x | | | | x | | x | | | | | x | | | | | | | x | | x |
| 938 | | | x | | | | x | | x | | | | | | x | | | | x | | | x | |
| 939 | | | x | | | | x | | x | | | | | | x | | | | x | | | | x |
| 940 | | | x | | | | x | | x | | | | | | x | | | | | x | | x | |
| 941 | | | x | | | | x | | x | | | | | | x | | | | | x | | | x |
| 942 | | | x | | | | x | | x | | | | | | x | | | | | | x | x | |
| 943 | | | x | | | | x | | x | | | | | | x | | | | | | x | | x |
| 944 | | | x | | | | x | | x | | | | | | | x | | | x | | | x | |
| 945 | | | x | | | | x | | x | | | | | | | x | | | x | | | | x |
| 946 | | | x | | | | x | | x | | | | | | | x | | | | x | | x | |
| 947 | | | x | | | | x | | x | | | | | | | x | | | | x | | | x |
| 948 | | | x | | | | x | | x | | | | | | | x | | | | | x | x | |
| 949 | | | x | | | | x | | x | | | | | | | x | | | | | x | | x |
| 950 | | | x | | | | x | | x | | | | | | | | x | | x | | | x | |
| 951 | | | x | | | | x | | x | | | | | | | | x | | x | | | | x |
| 952 | | | x | | | | x | | x | | | | | | | | x | | | x | | x | |
| 953 | | | x | | | | x | | x | | | | | | | | x | | | x | | | x |
| 954 | | | x | | | | x | | x | | | | | | | | x | | | | x | x | |
| 955 | | | x | | | | x | | x | | | | | | | | x | | | | x | | x |
| 956 | | | x | | | | x | | x | | | | | | | | | x | x | | | x | |
| 957 | | | x | | | | x | | x | | | | | | | | | x | x | | | | x |
| 958 | | | x | | | | x | | x | | | | | | | | | x | | x | | x | |
| 959 | | | x | | | | x | | x | | | | | | | | | x | | x | | | x |
| 960 | | | x | | | | x | | x | | | | | | | | | x | | | x | x | |
| 961 | | | x | | | | x | | x | | | | | | | | | x | | | x | | x |
| 962 | | | x | | | | x | | | x | | | | x | | | | | x | | | x | |
| 963 | | | x | | | | x | | | x | | | | x | | | | | x | | | | x |
| 964 | | | x | | | | x | | | x | | | | x | | | | | | x | | x | |
| 965 | | | x | | | | x | | | x | | | | x | | | | | | x | | | x |
| 966 | | | x | | | | x | | | x | | | | x | | | | | | | x | x | |
| 967 | | | x | | | | x | | | x | | | | x | | | | | | | x | | x |
| 968 | | | x | | | | x | | | x | | | | | x | | | | x | | | x | |
| 969 | | | x | | | | x | | | x | | | | | x | | | | x | | | | x |
| 970 | | | x | | | | x | | | x | | | | | x | | | | | x | | x | |
| 971 | | | x | | | | x | | | x | | | | | x | | | | | x | | | x |
| 972 | | | x | | | | x | | | x | | | | | x | | | | | | x | x | |
| 973 | | | x | | | | x | | | x | | | | | x | | | | | | x | | x |
| 974 | | | x | | | | x | | | x | | | | | | x | | | x | | | x | |
| 975 | | | x | | | | x | | | x | | | | | | x | | | x | | | | x |
| 976 | | | x | | | | x | | | x | | | | | | x | | | | x | | x | |
| 977 | | | x | | | | x | | | x | | | | | | x | | | | x | | | x |
| 978 | | | x | | | | x | | | x | | | | | | x | | | | | x | x | |
| 979 | | | x | | | | x | | | x | | | | | | x | | | | | x | | x |
| 980 | | | x | | | | x | | | x | | | | | | | x | | x | | | x | |
| 981 | | | x | | | | x | | | x | | | | | | | x | | x | | | | x |
| 982 | | | x | | | | x | | | x | | | | | | | x | | | x | | x | |
| 983 | | | x | | | | x | | | x | | | | | | | x | | | x | | | x |
| 984 | | | x | | | | x | | | x | | | | | | | x | | | | x | x | |
| 985 | | | x | | | | x | | | x | | | | | | | x | | | | x | | x |
| 986 | | | x | | | | x | | | x | | | | | | | | x | x | | | x | |
| 987 | | | x | | | | x | | | x | | | | | | | | x | x | | | | x |
| 988 | | | x | | | | x | | | x | | | | | | | | x | | x | | x | |
| 989 | | | x | | | | x | | | x | | | | | | | | x | | x | | | x |
| 990 | | | x | | | | x | | | x | | | | | | | | x | | | x | x | |
| 991 | | | x | | | | x | | | x | | | | | | | | x | | | x | | x |
| 992 | | | x | | | | x | | | | x | | | x | | | | | x | | | x | |
| 993 | | | x | | | | x | | | | x | | | x | | | | | x | | | | x |
| 994 | | | x | | | | x | | | | x | | | x | | | | | | x | | x | |
| 995 | | | x | | | | x | | | | x | | | x | | | | | | x | | | x |
| 996 | | | x | | | | x | | | | x | | | x | | | | | | | x | x | |
| 997 | | | x | | | | x | | | | x | | | x | | | | | | | x | | x |
| 998 | | | x | | | | x | | | | x | | | | x | | | | x | | | x | |
| 999 | | | x | | | | x | | | | x | | | | x | | | | x | | | | x |
| 1000 | | | x | | | | x | | | | x | | | | x | | | | | x | | x | |
| 1001 | | | x | | | | x | | | | x | | | | x | | | | | x | | | x |
| 1002 | | | x | | | | x | | | | x | | | | x | | | | | | x | x | |
| 1003 | | | x | | | | x | | | | x | | | | x | | | | | | x | | x |
| 1004 | | | x | | | | x | | | | x | | | | | x | | | x | | | x | |
| 1005 | | | x | | | | x | | | | x | | | | | x | | | x | | | | x |
| 1006 | | | x | | | | x | | | | x | | | | | x | | | | x | | x | |
| 1007 | | | x | | | | x | | | | x | | | | | x | | | | x | | | x |
| 1008 | | | x | | | | x | | | | x | | | | | x | | | | | x | x | |
| 1009 | | | x | | | | x | | | | x | | | | | x | | | | | x | | x |
| 1010 | | | x | | | | x | | | | x | | | | | | x | | x | | | x | |
| 1011 | | | x | | | | x | | | | x | | | | | | x | | x | | | | x |
| 1012 | | | x | | | | x | | | | x | | | | | | x | | | x | | x | |
| 1013 | | | x | | | | x | | | | x | | | | | | x | | | x | | | x |
| 1014 | | | x | | | | x | | | | x | | | | | | x | | | | x | x | |
| 1015 | | | x | | | | x | | | | x | | | | | | x | | | | x | | x |
| 1016 | | | x | | | | x | | | | x | | | | | | | x | x | | | x | |
| 1017 | | | x | | | | x | | | | x | | | | | | | x | x | | | | x |
| 1018 | | | x | | | | x | | | | x | | | | | | | x | | x | | x | |
| 1019 | | | x | | | | x | | | | x | | | | | | | x | | x | | | x |
| 1020 | | | x | | | | x | | | | x | | | | | | | x | | | x | x | |
| 1021 | | | x | | | | x | | | | x | | | | | | | x | | | x | | x |
| 1022 | | | x | | | | x | | | | | x | | x | | | | | x | | | x | |
| 1023 | | | x | | | | x | | | | | x | | x | | | | | x | | | | x |
| 1024 | | | x | | | | x | | | | | x | | x | | | | | | x | | x | |
| 1025 | | | x | | | | x | | | | | x | | x | | | | | | x | | | x |
| 1026 | | | x | | | | x | | | | | x | | x | | | | | | | x | x | |
| 1027 | | | x | | | | x | | | | | x | | x | | | | | | | x | | x |
| 1028 | | | x | | | | x | | | | | x | | | x | | | | x | | | x | |
| 1029 | | | x | | | | x | | | | | x | | | x | | | | x | | | | x |
| 1030 | | | x | | | | x | | | | | x | | | x | | | | | x | | x | |
| 1031 | | | x | | | | x | | | | | x | | | x | | | | | x | | | x |
| 1032 | | | x | | | | x | | | | | x | | | x | | | | | | x | x | |
| 1033 | | | x | | | | x | | | | | x | | | x | | | | | | x | | x |
| 1034 | | | x | | | | x | | | | | x | | | | x | | | x | | | x | |
| 1035 | | | x | | | | x | | | | | x | | | | x | | | x | | | | x |
| 1036 | | | x | | | | x | | | | | x | | | | x | | | | x | | x | |
| 1037 | | | x | | | | x | | | | | x | | | | x | | | | x | | | x |
| 1038 | | | x | | | | x | | | | | x | | | | x | | | | | x | x | |
| 1039 | | | x | | | | x | | | | | x | | | | x | | | | | x | | x |
| 1040 | | | x | | | | x | | | | | x | | | | | x | | x | | | x | |
| 1041 | | | x | | | | x | | | | | x | | | | | x | | x | | | | x |
| 1042 | | | x | | | | x | | | | | x | | | | | x | | | x | | x | |
| 1043 | | | x | | | | x | | | | | x | | | | | x | | | x | | | x |
| 1044 | | | x | | | | x | | | | | x | | | | | x | | | | x | x | |
| 1045 | | | x | | | | x | | | | | x | | | | | x | | | | x | | x |
| 1046 | | | x | | | | x | | | | | x | | | | | | x | x | | | x | |
| 1047 | | | x | | | | x | | | | | x | | | | | | x | x | | | | x |
| 1048 | | | x | | | | x | | | | | x | | | | | | x | | x | | x | |
| 1049 | | | x | | | | x | | | | | x | | | | | | x | | x | | | x |
| 1050 | | | x | | | | x | | | | | x | | | | | | x | | | x | x | |
| 1051 | | | x | | | | x | | | | | x | | | | | | x | | | x | | x |
| 1052 | | | x | | | | x | | | | | | x | x | | | | | x | | | x | |
| 1053 | | | x | | | | x | | | | | | x | x | | | | | x | | | | x |
| 1054 | | | x | | | | x | | | | | | x | x | | | | | | x | | x | |
| 1055 | | | x | | | | x | | | | | | x | x | | | | | | x | | | x |
| 1056 | | | x | | | | x | | | | | | x | x | | | | | | | x | x | |
| 1057 | | | x | | | | x | | | | | | x | x | | | | | | | x | | x |
| 1058 | | | x | | | | x | | | | | | x | | x | | | | x | | | x | |
| 1059 | | | x | | | | x | | | | | | x | | x | | | | x | | | | x |
| 1060 | | | x | | | | x | | | | | | x | | x | | | | | x | | x | |
| 1061 | | | x | | | | x | | | | | | x | | x | | | | | x | | | x |
| 1062 | | | x | | | | x | | | | | | x | | x | | | | | | x | x | |
| 1063 | | | x | | | | x | | | | | | x | | x | | | | | | x | | x |
| 1064 | | | x | | | | x | | | | | | x | | | x | | | x | | | x | |
| 1065 | | | x | | | | x | | | | | | x | | | x | | | x | | | | x |
| 1066 | | | x | | | | x | | | | | | x | | | x | | | | x | | x | |
| 1067 | | | x | | | | x | | | | | | x | | | x | | | | x | | | x |
| 1068 | | | x | | | | x | | | | | | x | | | x | | | | | x | x | |
| 1069 | | | x | | | | x | | | | | | x | | | x | | | | | x | | x |
| 1070 | | | x | | | | x | | | | | | x | | | | x | | x | | | x | |
| 1071 | | | x | | | | x | | | | | | x | | | | x | | x | | | | x |
| 1072 | | | x | | | | x | | | | | | x | | | | x | | | x | | x | |
| 1073 | | | x | | | | x | | | | | | x | | | | x | | | x | | | x |
| 1074 | | | x | | | | x | | | | | | x | | | | x | | | | x | x | |
| 1075 | | | x | | | | x | | | | | | x | | | | x | | | | x | | x |
| 1076 | | | x | | | | x | | | | | | x | | | | | x | x | | | x | |
| 1077 | | | x | | | | x | | | | | | x | | | | | x | x | | | | x |
| 1078 | | | x | | | | x | | | | | | x | | | | | x | | x | | x | |
| 1079 | | | x | | | | x | | | | | | x | | | | | x | | x | | | x |
| 1080 | | | x | | | | x | | | | | | x | | | | | x | | | x | x | |
| 1081 | | | x | | | | x | | | | | | x | | | | | x | | | x | | x |
| 1082 | | | | x | | x | | x | | | | | | x | | | | | x | | | x | |
| 1083 | | | | x | | x | | x | | | | | | x | | | | | x | | | | x |
| 1084 | | | | x | | x | | x | | | | | | x | | | | | | x | | x | |
| 1085 | | | | x | | x | | x | | | | | | x | | | | | | x | | | x |
| 1086 | | | | x | | x | | x | | | | | | x | | | | | | | x | x | |
| 1087 | | | | x | | x | | x | | | | | | x | | | | | | | x | | x |
| 1088 | | | | x | | x | | x | | | | | | | x | | | | x | | | x | |
| 1089 | | | | x | | x | | x | | | | | | | x | | | | x | | | | x |
| 1090 | | | | x | | x | | x | | | | | | | x | | | | | x | | x | |
| 1091 | | | | x | | x | | x | | | | | | | x | | | | | x | | | x |
| 1092 | | | | x | | x | | x | | | | | | | x | | | | | | x | x | |
| 1093 | | | | x | | x | | x | | | | | | | x | | | | | | x | | x |
| 1094 | | | | x | | x | | x | | | | | | | | x | | | x | | | x | |
| 1095 | | | | x | | x | | x | | | | | | | | x | | | x | | | | x |
| 1096 | | | | x | | x | | x | | | | | | | | x | | | | x | | x | |
| 1097 | | | | x | | x | | x | | | | | | | | x | | | | x | | | x |
| 1098 | | | | x | | x | | x | | | | | | | | x | | | | | x | x | |
| 1099 | | | | x | | x | | x | | | | | | | | x | | | | | x | | x |
| 1100 | | | | x | | x | | x | | | | | | | | | x | | x | | | x | |
| 1101 | | | | x | | x | | x | | | | | | | | | x | | x | | | | x |
| 1102 | | | | x | | x | | x | | | | | | | | | x | | | x | | x | |
| 1103 | | | | x | | x | | x | | | | | | | | | x | | | x | | | x |
| 1104 | | | | x | | x | | x | | | | | | | | | x | | | | x | x | |
| 1105 | | | | x | | x | | x | | | | | | | | | x | | | | x | | x |
| 1106 | | | | x | | x | | x | | | | | | | | | | x | x | | | x | |
| 1107 | | | | x | | x | | x | | | | | | | | | | x | x | | | | x |
| 1108 | | | | x | | x | | x | | | | | | | | | | x | | x | | x | |
| 1109 | | | | x | | x | | x | | | | | | | | | | x | | x | | | x |
| 1110 | | | | x | | x | | x | | | | | | | | | | x | | | x | x | |
| 1111 | | | | x | | x | | x | | | | | | | | | | x | | | x | | x |
| 1112 | | | | x | | x | | | x | | | | | x | | | | | x | | | x | |
| 1113 | | | | x | | x | | | x | | | | | x | | | | | x | | | | x |
| 1114 | | | | x | | x | | | x | | | | | x | | | | | | x | | x | |
| 1115 | | | | x | | x | | | x | | | | | x | | | | | | x | | | x |
| 1116 | | | | x | | x | | | x | | | | | x | | | | | | | x | x | |
| 1117 | | | | x | | x | | | x | | | | | x | | | | | | | x | | x |
| 1118 | | | | x | | x | | | x | | | | | | x | | | | x | | | x | |
| 1119 | | | | x | | x | | | x | | | | | | x | | | | x | | | | x |
| 1120 | | | | x | | x | | | x | | | | | | x | | | | | x | | x | |
| 1121 | | | | x | | x | | | x | | | | | | x | | | | | x | | | x |
| 1122 | | | | x | | x | | | x | | | | | | x | | | | | | x | x | |
| 1123 | | | | x | | x | | | x | | | | | | x | | | | | | x | | x |
| 1124 | | | | x | | x | | | x | | | | | | | x | | | x | | | x | |
| 1125 | | | | x | | x | | | x | | | | | | | x | | | x | | | | x |
| 1126 | | | | x | | x | | | x | | | | | | | x | | | | x | | x | |
| 1127 | | | | x | | x | | | x | | | | | | | x | | | | x | | | x |
| 1128 | | | | x | | x | | | x | | | | | | | x | | | | | x | x | |
| 1129 | | | | x | | x | | | x | | | | | | | x | | | | | x | | x |
| 1130 | | | | x | | x | | | x | | | | | | | | x | | x | | | x | |
| 1131 | | | | x | | x | | | x | | | | | | | | x | | x | | | | x |
| 1132 | | | | x | | x | | | x | | | | | | | | x | | | x | | x | |
| 1133 | | | | x | | x | | | x | | | | | | | | x | | | x | | | x |
| 1134 | | | | x | | x | | | x | | | | | | | | x | | | | x | x | |
| 1135 | | | | x | | x | | | x | | | | | | | | x | | | | x | | x |
| 1136 | | | | x | | x | | | x | | | | | | | | | x | x | | | x | |
| 1137 | | | | x | | x | | | x | | | | | | | | | x | x | | | | x |
| 1138 | | | | x | | x | | | x | | | | | | | | | x | | x | | x | |
| 1139 | | | | x | | x | | | x | | | | | | | | | x | | x | | | x |
| 1140 | | | | x | | x | | | x | | | | | | | | | x | | | x | x | |
| 1141 | | | | x | | x | | | x | | | | | | | | | x | | | x | | x |
| 1142 | | | | x | | x | | | | x | | | | x | | | | | x | | | x | |
| 1143 | | | | x | | x | | | | x | | | | x | | | | | x | | | | x |
| 1144 | | | | x | | x | | | | x | | | | x | | | | | | x | | x | |
| 1145 | | | | x | | x | | | | x | | | | x | | | | | | x | | | x |
| 1146 | | | | x | | x | | | | x | | | | x | | | | | | | x | x | |
| 1147 | | | | x | | x | | | | x | | | | x | | | | | | | x | | x |
| 1148 | | | | x | | x | | | | x | | | | | x | | | | x | | | x | |
| 1149 | | | | x | | x | | | | x | | | | | x | | | | x | | | | x |
| 1150 | | | | x | | x | | | | x | | | | | x | | | | | x | | x | |
| 1151 | | | | x | | x | | | | x | | | | | x | | | | | x | | | x |
| 1152 | | | | x | | x | | | | x | | | | | x | | | | | | x | x | |
| 1153 | | | | x | | x | | | | x | | | | | x | | | | | | x | | x |
| 1154 | | | | x | | x | | | | x | | | | | | x | | | x | | | x | |
| 1155 | | | | x | | x | | | | x | | | | | | x | | | x | | | | x |
| 1156 | | | | x | | x | | | | x | | | | | | x | | | | x | | x | |
| 1157 | | | | x | | x | | | | x | | | | | | x | | | | x | | | x |
| 1158 | | | | x | | x | | | | x | | | | | | x | | | | | x | x | |
| 1159 | | | | x | | x | | | | x | | | | | | x | | | | | x | | x |
| 1160 | | | | x | | x | | | | x | | | | | | | x | | x | | | x | |
| 1161 | | | | x | | x | | | | x | | | | | | | x | | x | | | | x |
| 1162 | | | | x | | x | | | | x | | | | | | | x | | | x | | x | |
| 1163 | | | | x | | x | | | | x | | | | | | | x | | | x | | | x |
| 1164 | | | | x | | x | | | | x | | | | | | | x | | | | x | x | |
| 1165 | | | | x | | x | | | | x | | | | | | | x | | | | x | | x |
| 1166 | | | | x | | x | | | | x | | | | | | | | x | x | | | x | |
| 1167 | | | | x | | x | | | | x | | | | | | | | x | x | | | | x |
| 1168 | | | | x | | x | | | | x | | | | | | | | x | | x | | x | |
| 1169 | | | | x | | x | | | | x | | | | | | | | x | | x | | | x |
| 1170 | | | | x | | x | | | | x | | | | | | | | x | | | x | x | |
| 1171 | | | | x | | x | | | | x | | | | | | | | x | | | x | | x |
| 1172 | | | | x | | x | | | | | x | | | x | | | | | x | | | x | |
| 1173 | | | | x | | x | | | | | x | | | x | | | | | x | | | | x |
| 1174 | | | | x | | x | | | | | x | | | x | | | | | | x | | x | |
| 1175 | | | | x | | x | | | | | x | | | x | | | | | | x | | | x |
| 1176 | | | | x | | x | | | | | x | | | x | | | | | | | x | x | |
| 1177 | | | | x | | x | | | | | x | | | x | | | | | | | x | | x |
| 1178 | | | | x | | x | | | | | x | | | | x | | | | x | | | x | |
| 1179 | | | | x | | x | | | | | x | | | | x | | | | x | | | | x |
| 1180 | | | | x | | x | | | | | x | | | | x | | | | | x | | x | |
| 1181 | | | | x | | x | | | | | x | | | | x | | | | | x | | | x |
| 1182 | | | | x | | x | | | | | x | | | | x | | | | | | x | x | |
| 1183 | | | | x | | x | | | | | x | | | | x | | | | | | x | | x |
| 1184 | | | | x | | x | | | | | x | | | | | x | | | x | | | x | |
| 1185 | | | | x | | x | | | | | x | | | | | x | | | x | | | | x |
| 1186 | | | | x | | x | | | | | x | | | | | x | | | | x | | x | |
| 1187 | | | | x | | x | | | | | x | | | | | x | | | | x | | | x |
| 1188 | | | | x | | x | | | | | x | | | | | x | | | | | x | x | |
| 1189 | | | | x | | x | | | | | x | | | | | x | | | | | x | | x |
| 1190 | | | | x | | x | | | | | x | | | | | | x | | x | | | x | |
| 1191 | | | | x | | x | | | | | x | | | | | | x | | x | | | | x |
| 1192 | | | | x | | x | | | | | x | | | | | | x | | | x | | x | |
| 1193 | | | | x | | x | | | | | x | | | | | | x | | | x | | | x |
| 1194 | | | | x | | x | | | | | x | | | | | | x | | | | x | x | |
| 1195 | | | | x | | x | | | | | x | | | | | | x | | | | x | | x |
| 1196 | | | | x | | x | | | | | x | | | | | | | x | x | | | x | |
| 1197 | | | | x | | x | | | | | x | | | | | | | x | x | | | | x |
| 1198 | | | | x | | x | | | | | x | | | | | | | x | | x | | x | |
| 1199 | | | | x | | x | | | | | x | | | | | | | x | | x | | | x |
| 1200 | | | | x | | x | | | | | x | | | | | | | x | | | x | x | |
| 1201 | | | | x | | x | | | | | x | | | | | | | x | | | x | | x |
| 1202 | | | | x | | x | | | | | | x | | x | | | | | x | | | x | |
| 1203 | | | | x | | x | | | | | | x | | x | | | | | x | | | | x |
| 1204 | | | | x | | x | | | | | | x | | x | | | | | | x | | x | |
| 1205 | | | | x | | x | | | | | | x | | x | | | | | | x | | | x |
| 1206 | | | | x | | x | | | | | | x | | x | | | | | | | x | x | |
| 1207 | | | | x | | x | | | | | | x | | x | | | | | | | x | | x |
| 1208 | | | | x | | x | | | | | | x | | | x | | | | x | | | x | |
| 1209 | | | | x | | x | | | | | | x | | | x | | | | x | | | | x |
| 1210 | | | | x | | x | | | | | | x | | | x | | | | | x | | x | |
| 1211 | | | | x | | x | | | | | | x | | | x | | | | | x | | | x |
| 1212 | | | | x | | x | | | | | | x | | | x | | | | | | x | x | |
| 1213 | | | | x | | x | | | | | | x | | | x | | | | | | x | | x |
| 1214 | | | | x | | x | | | | | | x | | | | x | | | x | | | x | |
| 1215 | | | | x | | x | | | | | | x | | | | x | | | x | | | | x |
| 1216 | | | | x | | x | | | | | | x | | | | x | | | | x | | x | |
| 1217 | | | | x | | x | | | | | | x | | | | x | | | | x | | | x |
| 1218 | | | | x | | x | | | | | | x | | | | x | | | | | x | x | |
| 1219 | | | | x | | x | | | | | | x | | | | x | | | | | x | | x |
| 1220 | | | | x | | x | | | | | | x | | | | | x | | x | | | x | |
| 1221 | | | | x | | x | | | | | | x | | | | | x | | x | | | | x |
| 1222 | | | | x | | x | | | | | | x | | | | | x | | | x | | x | |
| 1223 | | | | x | | x | | | | | | x | | | | | x | | | x | | | x |
| 1224 | | | | x | | x | | | | | | x | | | | | x | | | | x | x | |
| 1225 | | | | x | | x | | | | | | x | | | | | x | | | | x | | x |
| 1226 | | | | x | | x | | | | | | x | | | | | | x | x | | | x | |
| 1227 | | | | x | | x | | | | | | x | | | | | | x | x | | | | x |
| 1228 | | | | x | | x | | | | | | x | | | | | | x | | x | | x | |
| 1229 | | | | x | | x | | | | | | x | | | | | | x | | x | | | x |
| 1230 | | | | x | | x | | | | | | x | | | | | | x | | | x | x | |
| 1231 | | | | x | | x | | | | | | x | | | | | | x | | | x | | x |
| 1232 | | | | x | | x | | | | | | | x | x | | | | | x | | | x | |
| 1233 | | | | x | | x | | | | | | | x | x | | | | | x | | | | x |
| 1234 | | | | x | | x | | | | | | | x | x | | | | | | x | | x | |
| 1235 | | | | x | | x | | | | | | | x | x | | | | | | x | | | x |
| 1236 | | | | x | | x | | | | | | | x | x | | | | | | | x | x | |
| 1237 | | | | x | | x | | | | | | | x | x | | | | | | | x | | x |
| 1238 | | | | x | | x | | | | | | | x | | x | | | | x | | | x | |
| 1239 | | | | x | | x | | | | | | | x | | x | | | | x | | | | x |
| 1240 | | | | x | | x | | | | | | | x | | x | | | | | x | | x | |
| 1241 | | | | x | | x | | | | | | | x | | x | | | | | x | | | x |
| 1242 | | | | x | | x | | | | | | | x | | x | | | | | | x | x | |
| 1243 | | | | x | | x | | | | | | | x | | x | | | | | | x | | x |
| 1244 | | | | x | | x | | | | | | | x | | | x | | | x | | | x | |
| 1245 | | | | x | | x | | | | | | | x | | | x | | | x | | | | x |
| 1246 | | | | x | | x | | | | | | | x | | | x | | | | x | | x | |
| 1247 | | | | x | | x | | | | | | | x | | | x | | | | x | | | x |
| 1248 | | | | x | | x | | | | | | | x | | | x | | | | | x | x | |
| 1249 | | | | x | | x | | | | | | | x | | | x | | | | | x | | x |
| 1250 | | | | x | | x | | | | | | | x | | | | x | | x | | | x | |
| 1251 | | | | x | | x | | | | | | | x | | | | x | | x | | | | x |
| 1252 | | | | x | | x | | | | | | | x | | | | x | | | x | | x | |
| 1253 | | | | x | | x | | | | | | | x | | | | x | | | x | | | x |
| 1254 | | | | x | | x | | | | | | | x | | | | x | | | | x | x | |
| 1255 | | | | x | | x | | | | | | | x | | | | x | | | | x | | x |
| 1256 | | | | x | | x | | | | | | | x | | | | | x | x | | | x | |
| 1257 | | | | x | | x | | | | | | | x | | | | | x | x | | | | x |
| 1258 | | | | x | | x | | | | | | | x | | | | | x | | x | | x | |
| 1259 | | | | x | | x | | | | | | | x | | | | | x | | x | | | x |
| 1260 | | | | x | | x | | | | | | | x | | | | | x | | | x | x | |
| 1261 | | | | x | | x | | | | | | | x | | | | | x | | | x | | x |
| 1262 | | | | x | | | x | x | | | | | | x | | | | | x | | | x | |
| 1263 | | | | x | | | x | x | | | | | | x | | | | | x | | | | x |
| 1264 | | | | x | | | x | x | | | | | | x | | | | | | x | | x | |
| 1265 | | | | x | | | x | x | | | | | | x | | | | | | x | | | x |
| 1266 | | | | x | | | x | x | | | | | | x | | | | | | | x | x | |
| 1267 | | | | x | | | x | x | | | | | | x | | | | | | | x | | x |
| 1268 | | | | x | | | x | x | | | | | | | x | | | | x | | | x | |
| 1269 | | | | x | | | x | x | | | | | | | x | | | | x | | | | x |
| 1270 | | | | x | | | x | x | | | | | | | x | | | | | x | | x | |
| 1271 | | | | x | | | x | x | | | | | | | x | | | | | x | | | x |
| 1272 | | | | x | | | x | x | | | | | | | x | | | | | | x | x | |
| 1273 | | | | x | | | x | x | | | | | | | x | | | | | | x | | x |
| 1274 | | | | x | | | x | x | | | | | | | | x | | | x | | | x | |
| 1275 | | | | x | | | x | x | | | | | | | | x | | | x | | | | x |
| 1276 | | | | x | | | x | x | | | | | | | | x | | | | x | | x | |
| 1277 | | | | x | | | x | x | | | | | | | | x | | | | x | | | x |
| 1278 | | | | x | | | x | x | | | | | | | | x | | | | | x | x | |
| 1279 | | | | x | | | x | x | | | | | | | | x | | | | | x | | x |
| 1280 | | | | x | | | x | x | | | | | | | | | x | | x | | | x | |
| 1281 | | | | x | | | x | x | | | | | | | | | x | | x | | | | x |
| 1282 | | | | x | | | x | x | | | | | | | | | x | | | x | | x | |
| 1283 | | | | x | | | x | x | | | | | | | | | x | | | x | | | x |
| 1284 | | | | x | | | x | x | | | | | | | | | x | | | | x | x | |
| 1285 | | | | x | | | x | x | | | | | | | | | x | | | | x | | x |
| 1286 | | | | x | | | x | x | | | | | | | | | | x | x | | | x | |
| 1287 | | | | x | | | x | x | | | | | | | | | | x | x | | | | x |
| 1288 | | | | x | | | x | x | | | | | | | | | | x | | x | | x | |
| 1289 | | | | x | | | x | x | | | | | | | | | | x | | x | | | x |
| 1290 | | | | x | | | x | x | | | | | | | | | | x | | | x | x | |
| 1291 | | | | x | | | x | x | | | | | | | | | | x | | | x | | x |
| 1292 | | | | x | | | x | | x | | | | | x | | | | | x | | | x | |
| 1293 | | | | x | | | x | | x | | | | | x | | | | | x | | | | x |
| 1294 | | | | x | | | x | | x | | | | | x | | | | | | x | | x | |
| 1295 | | | | x | | | x | | x | | | | | x | | | | | | x | | | x |
| 1296 | | | | x | | | x | | x | | | | | x | | | | | | | x | x | |
| 1297 | | | | x | | | x | | x | | | | | x | | | | | | | x | | x |
| 1298 | | | | x | | | x | | x | | | | | | x | | | | x | | | x | |
| 1299 | | | | x | | | x | | x | | | | | | x | | | | x | | | | x |
| 1300 | | | | x | | | x | | x | | | | | | x | | | | | x | | x | |
| 1301 | | | | x | | | x | | x | | | | | | x | | | | | x | | | x |
| 1302 | | | | x | | | x | | x | | | | | | x | | | | | | x | x | |
| 1303 | | | | x | | | x | | x | | | | | | x | | | | | | x | | x |
| 1304 | | | | x | | | x | | x | | | | | | | x | | | x | | | x | |
| 1305 | | | | x | | | x | | x | | | | | | | x | | | x | | | | x |
| 1306 | | | | x | | | x | | x | | | | | | | x | | | | x | | x | |
| 1307 | | | | x | | | x | | x | | | | | | | x | | | | x | | | x |
| 1308 | | | | x | | | x | | x | | | | | | | x | | | | | x | x | |
| 1309 | | | | x | | | x | | x | | | | | | | x | | | | | x | | x |
| 1310 | | | | x | | | x | | x | | | | | | | | x | | x | | | x | |
| 1311 | | | | x | | | x | | x | | | | | | | | x | | x | | | | x |
| 1312 | | | | x | | | x | | x | | | | | | | | x | | | x | | x | |
| 1313 | | | | x | | | x | | x | | | | | | | | x | | | x | | | x |
| 1314 | | | | x | | | x | | x | | | | | | | | x | | | | x | x | |
| 1315 | | | | x | | | x | | x | | | | | | | | x | | | | x | | x |
| 1316 | | | | x | | | x | | x | | | | | | | | | x | x | | | x | |
| 1317 | | | | x | | | x | | x | | | | | | | | | x | x | | | | x |
| 1318 | | | | x | | | x | | x | | | | | | | | | x | | x | | x | |
| 1319 | | | | x | | | x | | x | | | | | | | | | x | | x | | | x |
| 1320 | | | | x | | | x | | x | | | | | | | | | x | | | x | x | |
| 1321 | | | | x | | | x | | x | | | | | | | | | x | | | x | | x |
| 1322 | | | | x | | | x | | | x | | | | x | | | | | x | | | x | |
| 1323 | | | | x | | | x | | | x | | | | x | | | | | x | | | | x |
| 1324 | | | | x | | | x | | | x | | | | x | | | | | | x | | x | |
| 1325 | | | | x | | | x | | | x | | | | x | | | | | | x | | | x |
| 1326 | | | | x | | | x | | | x | | | | x | | | | | | | x | x | |
| 1327 | | | | x | | | x | | | x | | | | x | | | | | | | x | | x |
| 1328 | | | | x | | | x | | | x | | | | | x | | | | x | | | x | |
| 1329 | | | | x | | | x | | | x | | | | | x | | | | x | | | | x |
| 1330 | | | | x | | | x | | | x | | | | | x | | | | | x | | x | |
| 1331 | | | | x | | | x | | | x | | | | | x | | | | | x | | | x |
| 1332 | | | | x | | | x | | | x | | | | | x | | | | | | x | x | |
| 1333 | | | | x | | | x | | | x | | | | | x | | | | | | x | | x |
| 1334 | | | | x | | | x | | | x | | | | | | x | | | x | | | x | |
| 1335 | | | | x | | | x | | | x | | | | | | x | | | x | | | | x |
| 1336 | | | | x | | | x | | | x | | | | | | x | | | | x | | x | |
| 1337 | | | | x | | | x | | | x | | | | | | x | | | | x | | | x |
| 1338 | | | | x | | | x | | | x | | | | | | x | | | | | x | x | |
| 1339 | | | | x | | | x | | | x | | | | | | x | | | | | x | | x |
| 1340 | | | | x | | | x | | | x | | | | | | | x | | x | | | x | |
| 1341 | | | | x | | | x | | | x | | | | | | | x | | x | | | | x |
| 1342 | | | | x | | | x | | | x | | | | | | | x | | | x | | x | |
| 1343 | | | | x | | | x | | | x | | | | | | | x | | | x | | | x |
| 1344 | | | | x | | | x | | | x | | | | | | | x | | | | x | x | |
| 1345 | | | | x | | | x | | | x | | | | | | | x | | | | x | | x |
| 1346 | | | | x | | | x | | | x | | | | | | | | x | x | | | x | |
| 1347 | | | | x | | | x | | | x | | | | | | | | x | x | | | | x |
| 1348 | | | | x | | | x | | | x | | | | | | | | x | | x | | x | |
| 1349 | | | | x | | | x | | | x | | | | | | | | x | | x | | | x |
| 1350 | | | | x | | | x | | | x | | | | | | | | x | | | x | x | |
| 1351 | | | | x | | | x | | | x | | | | | | | | x | | | x | | x |
| 1352 | | | | x | | | x | | | | x | | | x | | | | | x | | | x | |
| 1353 | | | | x | | | x | | | | x | | | x | | | | | x | | | | x |
| 1354 | | | | x | | | x | | | | x | | | x | | | | | | x | | x | |
| 1355 | | | | x | | | x | | | | x | | | x | | | | | | x | | | x |
| 1356 | | | | x | | | x | | | | x | | | x | | | | | | | x | x | |
| 1357 | | | | x | | | x | | | | x | | | x | | | | | | | x | | x |
| 1358 | | | | x | | | x | | | | x | | | | x | | | | x | | | x | |
| 1359 | | | | x | | | x | | | | x | | | | x | | | | x | | | | x |
| 1360 | | | | x | | | x | | | | x | | | | x | | | | | x | | x | |
| 1361 | | | | x | | | x | | | | x | | | | x | | | | | x | | | x |
| 1362 | | | | x | | | x | | | | x | | | | x | | | | | | x | x | |
| 1363 | | | | x | | | x | | | | x | | | | x | | | | | | x | | x |
| 1364 | | | | x | | | x | | | | x | | | | | x | | | x | | | x | |
| 1365 | | | | x | | | x | | | | x | | | | | x | | | x | | | | x |
| 1366 | | | | x | | | x | | | | x | | | | | x | | | | x | | x | |
| 1367 | | | | x | | | x | | | | x | | | | | x | | | | x | | | x |
| 1368 | | | | x | | | x | | | | x | | | | | x | | | | | x | x | |
| 1369 | | | | x | | | x | | | | x | | | | | x | | | | | x | | x |
| 1370 | | | | x | | | x | | | | x | | | | | | x | | x | | | x | |
| 1371 | | | | x | | | x | | | | x | | | | | | x | | x | | | | x |
| 1372 | | | | x | | | x | | | | x | | | | | | x | | | x | | x | |
| 1373 | | | | x | | | x | | | | x | | | | | | x | | | x | | | x |
| 1374 | | | | x | | | x | | | | x | | | | | | x | | | | x | x | |
| 1375 | | | | x | | | x | | | | x | | | | | | x | | | | x | | x |
| 1376 | | | | x | | | x | | | | x | | | | | | | x | x | | | x | |
| 1377 | | | | x | | | x | | | | x | | | | | | | x | x | | | | x |
| 1378 | | | | x | | | x | | | | x | | | | | | | x | | x | | x | |
| 1379 | | | | x | | | x | | | | x | | | | | | | x | | x | | | x |
| 1380 | | | | x | | | x | | | | x | | | | | | | x | | | x | x | |
| 1381 | | | | x | | | x | | | | x | | | | | | | x | | | x | | x |
| 1382 | | | | x | | | x | | | | | x | | x | | | | | x | | | x | |
| 1383 | | | | x | | | x | | | | | x | | x | | | | | x | | | | x |
| 1384 | | | | x | | | x | | | | | x | | x | | | | | | x | | x | |
| 1385 | | | | x | | | x | | | | | x | | x | | | | | | x | | | x |
| 1386 | | | | x | | | x | | | | | x | | x | | | | | | | x | x | |
| 1387 | | | | x | | | x | | | | | x | | x | | | | | | | x | | x |
| 1388 | | | | x | | | x | | | | | x | | | x | | | | x | | | x | |
| 1389 | | | | x | | | x | | | | | x | | | x | | | | x | | | | x |
| 1390 | | | | x | | | x | | | | | x | | | x | | | | | x | | x | |
| 1391 | | | | x | | | x | | | | | x | | | x | | | | | x | | | x |
| 1392 | | | | x | | | x | | | | | x | | | x | | | | | | x | x | |
| 1393 | | | | x | | | x | | | | | x | | | x | | | | | | x | | x |
| 1394 | | | | x | | | x | | | | | x | | | | x | | | x | | | x | |
| 1395 | | | | x | | | x | | | | | x | | | | x | | | x | | | | x |
| 1396 | | | | x | | | x | | | | | x | | | | x | | | | x | | x | |
| 1397 | | | | x | | | x | | | | | x | | | | x | | | | x | | | x |
| 1398 | | | | x | | | x | | | | | x | | | | x | | | | | x | x | |
| 1399 | | | | x | | | x | | | | | x | | | | x | | | | | x | | x |
| 1400 | | | | x | | | x | | | | | x | | | | | x | | x | | | x | |
| 1401 | | | | x | | | x | | | | | x | | | | | x | | x | | | | x |
| 1402 | | | | x | | | x | | | | | x | | | | | x | | | x | | x | |
| 1403 | | | | x | | | x | | | | | x | | | | | x | | | x | | | x |
| 1404 | | | | x | | | x | | | | | x | | | | | x | | | | x | x | |
| 1405 | | | | x | | | x | | | | | x | | | | | x | | | | x | | x |
| 1406 | | | | x | | | x | | | | | x | | | | | | x | x | | | x | |
| 1407 | | | | x | | | x | | | | | x | | | | | | x | x | | | | x |
| 1408 | | | | x | | | x | | | | | x | | | | | | x | | x | | x | |
| 1409 | | | | x | | | x | | | | | x | | | | | | x | | x | | | x |
| 1410 | | | | x | | | x | | | | | x | | | | | | x | | | x | x | |
| 1411 | | | | x | | | x | | | | | x | | | | | | x | | | x | | x |
| 1412 | | | | x | | | x | | | | | | x | x | | | | | x | | | x | |
| 1413 | | | | x | | | x | | | | | | x | x | | | | | x | | | | x |
| 1414 | | | | x | | | x | | | | | | x | x | | | | | | x | | x | |
| 1415 | | | | x | | | x | | | | | | x | x | | | | | | x | | | x |
| 1416 | | | | x | | | x | | | | | | x | x | | | | | | | x | x | |
| 1417 | | | | x | | | x | | | | | | x | x | | | | | | | x | | x |
| 1418 | | | | x | | | x | | | | | | x | | x | | | | x | | | x | |
| 1419 | | | | x | | | x | | | | | | x | | x | | | | x | | | | x |
| 1420 | | | | x | | | x | | | | | | x | | x | | | | | x | | x | |
| 1421 | | | | x | | | x | | | | | | x | | x | | | | | x | | | x |
| 1422 | | | | x | | | x | | | | | | x | | x | | | | | | x | x | |
| 1423 | | | | x | | | x | | | | | | x | | x | | | | | | x | | x |
| 1424 | | | | x | | | x | | | | | | x | | | x | | | x | | | x | |
| 1425 | | | | x | | | x | | | | | | x | | | x | | | x | | | | x |
| 1426 | | | | x | | | x | | | | | | x | | | x | | | | x | | x | |
| 1427 | | | | x | | | x | | | | | | x | | | x | | | | x | | | x |
| 1428 | | | | x | | | x | | | | | | x | | | x | | | | | x | x | |
| 1429 | | | | x | | | x | | | | | | x | | | x | | | | | x | | x |
| 1430 | | | | x | | | x | | | | | | x | | | | x | | x | | | x | |
| 1431 | | | | x | | | x | | | | | | x | | | | x | | x | | | | x |
| 1432 | | | | x | | | x | | | | | | x | | | | x | | | x | | x | |
| 1433 | | | | x | | | x | | | | | | x | | | | x | | | x | | | x |
| 1434 | | | | x | | | x | | | | | | x | | | | x | | | | x | x | |
| 1435 | | | | x | | | x | | | | | | x | | | | x | | | | x | | x |
| 1436 | | | | x | | | x | | | | | | x | | | | | x | x | | | x | |
| 1437 | | | | x | | | x | | | | | | x | | | | | x | x | | | | x |
| 1438 | | | | x | | | x | | | | | | x | | | | | x | | x | | x | |
| 1439 | | | | x | | | x | | | | | | x | | | | | x | | x | | | x |
| 1440 | | | | x | | | x | | | | | | x | | | | | x | | | x | x | |
| 1441 | | | | x | | | x | | | | | | x | | | | | x | | | x | | x |
| 1442 | | | | | x | x | | x | | | | | | x | | | | | x | | | x | |
| 1443 | | | | | x | x | | x | | | | | | x | | | | | x | | | | x |
| 1444 | | | | | x | x | | x | | | | | | x | | | | | | x | | x | |
| 1445 | | | | | x | x | | x | | | | | | x | | | | | | x | | | x |
| 1446 | | | | | x | x | | x | | | | | | x | | | | | | | x | x | |
| 1447 | | | | | x | x | | x | | | | | | x | | | | | | | x | | x |
| 1448 | | | | | x | x | | x | | | | | | | x | | | | x | | | x | |
| 1449 | | | | | x | x | | x | | | | | | | x | | | | x | | | | x |
| 1450 | | | | | x | x | | x | | | | | | | x | | | | | x | | x | |
| 1451 | | | | | x | x | | x | | | | | | | x | | | | | x | | | x |
| 1452 | | | | | x | x | | x | | | | | | | x | | | | | | x | x | |
| 1453 | | | | | x | x | | x | | | | | | | x | | | | | | x | | x |
| 1454 | | | | | x | x | | x | | | | | | | | x | | | x | | | x | |
| 1455 | | | | | x | x | | x | | | | | | | | x | | | x | | | | x |
| 1456 | | | | | x | x | | x | | | | | | | | x | | | | x | | x | |
| 1457 | | | | | x | x | | x | | | | | | | | x | | | | x | | | x |
| 1458 | | | | | x | x | | x | | | | | | | | x | | | | | x | x | |
| 1459 | | | | | x | x | | x | | | | | | | | x | | | | | x | | x |
| 1460 | | | | | x | x | | x | | | | | | | | | x | | x | | | x | |
| 1461 | | | | | x | x | | x | | | | | | | | | x | | x | | | | x |
| 1462 | | | | | x | x | | x | | | | | | | | | x | | | x | | x | |
| 1463 | | | | | x | x | | x | | | | | | | | | x | | | x | | | x |
| 1464 | | | | | x | x | | x | | | | | | | | | x | | | | x | x | |
| 1465 | | | | | x | x | | x | | | | | | | | | x | | | | x | | x |
| 1466 | | | | | x | x | | x | | | | | | | | | | x | x | | | x | |
| 1467 | | | | | x | x | | x | | | | | | | | | | x | x | | | | x |
| 1468 | | | | | x | x | | x | | | | | | | | | | x | | x | | x | |
| 1469 | | | | | x | x | | x | | | | | | | | | | x | | x | | | x |
| 1470 | | | | | x | x | | x | | | | | | | | | | x | | | x | x | |
| 1471 | | | | | x | x | | x | | | | | | | | | | x | | | x | | x |
| 1472 | | | | | x | x | | | x | | | | | x | | | | | x | | | x | |
| 1473 | | | | | x | x | | | x | | | | | x | | | | | x | | | | x |
| 1474 | | | | | x | x | | | x | | | | | x | | | | | | x | | x | |
| 1475 | | | | | x | x | | | x | | | | | x | | | | | | x | | | x |
| 1476 | | | | | x | x | | | x | | | | | x | | | | | | | x | x | |
| 1477 | | | | | x | x | | | x | | | | | x | | | | | | | x | | x |
| 1478 | | | | | x | x | | | x | | | | | | x | | | | x | | | x | |
| 1479 | | | | | x | x | | | x | | | | | | x | | | | x | | | | x |
| 1480 | | | | | x | x | | | x | | | | | | x | | | | | x | | x | |
| 1481 | | | | | x | x | | | x | | | | | | x | | | | | x | | | x |
| 1482 | | | | | x | x | | | x | | | | | | x | | | | | | x | x | |
| 1483 | | | | | x | x | | | x | | | | | | x | | | | | | x | | x |
| 1484 | | | | | x | x | | | x | | | | | | | x | | | x | | | x | |
| 1485 | | | | | x | x | | | x | | | | | | | x | | | x | | | | x |
| 1486 | | | | | x | x | | | x | | | | | | | x | | | | x | | x | |
| 1487 | | | | | x | x | | | x | | | | | | | x | | | | x | | | x |
| 1488 | | | | | x | x | | | x | | | | | | | x | | | | | x | x | |
| 1489 | | | | | x | x | | | x | | | | | | | x | | | | | x | | x |
| 1490 | | | | | x | x | | | x | | | | | | | | x | | x | | | x | |
| 1491 | | | | | x | x | | | x | | | | | | | | x | | x | | | | x |
| 1492 | | | | | x | x | | | x | | | | | | | | x | | | x | | x | |
| 1493 | | | | | x | x | | | x | | | | | | | | x | | | x | | | x |
| 1494 | | | | | x | x | | | x | | | | | | | | x | | | | x | x | |
| 1495 | | | | | x | x | | | x | | | | | | | | x | | | | x | | x |
| 1496 | | | | | x | x | | | x | | | | | | | | | x | x | | | x | |
| 1497 | | | | | x | x | | | x | | | | | | | | | x | x | | | | x |
| 1498 | | | | | x | x | | | x | | | | | | | | | x | | x | | x | |
| 1499 | | | | | x | x | | | x | | | | | | | | | x | | x | | | x |
| 1500 | | | | | x | x | | | x | | | | | | | | | x | | | x | x | |
| 1501 | | | | | x | x | | | x | | | | | | | | | x | | | x | | x |
| 1502 | | | | | x | x | | | | x | | | | x | | | | | x | | | x | |
| 1503 | | | | | x | x | | | | x | | | | x | | | | | x | | | | x |
| 1504 | | | | | x | x | | | | x | | | | x | | | | | | x | | x | |
| 1505 | | | | | x | x | | | | x | | | | x | | | | | | x | | | x |
| 1506 | | | | | x | x | | | | x | | | | x | | | | | | | x | x | |
| 1507 | | | | | x | x | | | | x | | | | x | | | | | | | x | | x |
| 1508 | | | | | x | x | | | | x | | | | | x | | | | x | | | x | |
| 1509 | | | | | x | x | | | | x | | | | | x | | | | x | | | | x |
| 1510 | | | | | x | x | | | | x | | | | | x | | | | | x | | x | |
| 1511 | | | | | x | x | | | | x | | | | | x | | | | | x | | | x |
| 1512 | | | | | x | x | | | | x | | | | | x | | | | | | x | x | |
| 1513 | | | | | x | x | | | | x | | | | | x | | | | | | x | | x |
| 1514 | | | | | x | x | | | | x | | | | | | x | | | x | | | x | |
| 1515 | | | | | x | x | | | | x | | | | | | x | | | x | | | | x |
| 1516 | | | | | x | x | | | | x | | | | | | x | | | | x | | x | |
| 1517 | | | | | x | x | | | | x | | | | | | x | | | | x | | | x |
| 1518 | | | | | x | x | | | | x | | | | | | x | | | | | x | x | |
| 1519 | | | | | x | x | | | | x | | | | | | x | | | | | x | | x |
| 1520 | | | | | x | x | | | | x | | | | | | | x | | x | | | x | |
| 1521 | | | | | x | x | | | | x | | | | | | | x | | x | | | | x |
| 1522 | | | | | x | x | | | | x | | | | | | | x | | | x | | x | |
| 1523 | | | | | x | x | | | | x | | | | | | | x | | | x | | | x |
| 1524 | | | | | x | x | | | | x | | | | | | | x | | | | x | x | |
| 1525 | | | | | x | x | | | | x | | | | | | | x | | | | x | | x |
| 1526 | | | | | x | x | | | | x | | | | | | | | x | x | | | x | |
| 1527 | | | | | x | x | | | | x | | | | | | | | x | x | | | | x |
| 1528 | | | | | x | x | | | | x | | | | | | | | x | | x | | x | |
| 1529 | | | | | x | x | | | | x | | | | | | | | x | | x | | | x |
| 1530 | | | | | x | x | | | | x | | | | | | | | x | | | x | x | |
| 1531 | | | | | x | x | | | | x | | | | | | | | x | | | x | | x |
| 1532 | | | | | x | x | | | | | x | | | x | | | | | x | | | x | |
| 1533 | | | | | x | x | | | | | x | | | x | | | | | x | | | | x |
| 1534 | | | | | x | x | | | | | x | | | x | | | | | | x | | x | |
| 1535 | | | | | x | x | | | | | x | | | x | | | | | | x | | | x |
| 1536 | | | | | x | x | | | | | x | | | x | | | | | | | x | x | |
| 1537 | | | | | x | x | | | | | x | | | x | | | | | | | x | | x |
| 1538 | | | | | x | x | | | | | x | | | | x | | | | x | | | x | |
| 1539 | | | | | x | x | | | | | x | | | | x | | | | x | | | | x |
| 1540 | | | | | x | x | | | | | x | | | | x | | | | | x | | x | |
| 1541 | | | | | x | x | | | | | x | | | | x | | | | | x | | | x |
| 1542 | | | | | x | x | | | | | x | | | | x | | | | | | x | x | |
| 1543 | | | | | x | x | | | | | x | | | | x | | | | | | x | | x |
| 1544 | | | | | x | x | | | | | x | | | | | x | | | x | | | x | |
| 1545 | | | | | x | x | | | | | x | | | | | x | | | x | | | | x |
| 1546 | | | | | x | x | | | | | x | | | | | x | | | | x | | x | |
| 1547 | | | | | x | x | | | | | x | | | | | x | | | | x | | | x |
| 1548 | | | | | x | x | | | | | x | | | | | x | | | | | x | x | |
| 1549 | | | | | x | x | | | | | x | | | | | x | | | | | x | | x |
| 1550 | | | | | x | x | | | | | x | | | | | | x | | x | | | x | |
| 1551 | | | | | x | x | | | | | x | | | | | | x | | x | | | | x |
| 1552 | | | | | x | x | | | | | x | | | | | | x | | | x | | x | |
| 1553 | | | | | x | x | | | | | x | | | | | | x | | | x | | | x |
| 1554 | | | | | x | x | | | | | x | | | | | | x | | | | x | x | |
| 1555 | | | | | x | x | | | | | x | | | | | | x | | | | x | | x |
| 1556 | | | | | x | x | | | | | x | | | | | | | x | x | | | x | |
| 1557 | | | | | x | x | | | | | x | | | | | | | x | x | | | | x |
| 1558 | | | | | x | x | | | | | x | | | | | | | x | | x | | x | |
| 1559 | | | | | x | x | | | | | x | | | | | | | x | | x | | | x |
| 1560 | | | | | x | x | | | | | x | | | | | | | x | | | x | x | |
| 1561 | | | | | x | x | | | | | x | | | | | | | x | | | x | | x |
| 1562 | | | | | x | x | | | | | | x | | x | | | | | x | | | x | |
| 1563 | | | | | x | x | | | | | | x | | x | | | | | x | | | | x |
| 1564 | | | | | x | x | | | | | | x | | x | | | | | | x | | x | |
| 1565 | | | | | x | x | | | | | | x | | x | | | | | | x | | | x |
| 1566 | | | | | x | x | | | | | | x | | x | | | | | | | x | x | |
| 1567 | | | | | x | x | | | | | | x | | x | | | | | | | x | | x |
| 1568 | | | | | x | x | | | | | | x | | | x | | | | x | | | x | |
| 1569 | | | | | x | x | | | | | | x | | | x | | | | x | | | | x |
| 1570 | | | | | x | x | | | | | | x | | | x | | | | | x | | x | |
| 1571 | | | | | x | x | | | | | | x | | | x | | | | | x | | | x |
| 1572 | | | | | x | x | | | | | | x | | | x | | | | | | x | x | |
| 1573 | | | | | x | x | | | | | | x | | | x | | | | | | x | | x |
| 1574 | | | | | x | x | | | | | | x | | | | x | | | x | | | x | |
| 1575 | | | | | x | x | | | | | | x | | | | x | | | x | | | | x |
| 1576 | | | | | x | x | | | | | | x | | | | x | | | | x | | x | |
| 1577 | | | | | x | x | | | | | | x | | | | x | | | | x | | | x |
| 1578 | | | | | x | x | | | | | | x | | | | x | | | | | x | x | |
| 1579 | | | | | x | x | | | | | | x | | | | x | | | | | x | | x |
| 1580 | | | | | x | x | | | | | | x | | | | | x | | x | | | x | |
| 1581 | | | | | x | x | | | | | | x | | | | | x | | x | | | | x |
| 1582 | | | | | x | x | | | | | | x | | | | | x | | | x | | x | |
| 1583 | | | | | x | x | | | | | | x | | | | | x | | | x | | | x |
| 1584 | | | | | x | x | | | | | | x | | | | | x | | | | x | x | |
| 1585 | | | | | x | x | | | | | | x | | | | | x | | | | x | | x |
| 1586 | | | | | x | x | | | | | | x | | | | | | x | x | | | x | |
| 1587 | | | | | x | x | | | | | | x | | | | | | x | x | | | | x |
| 1588 | | | | | x | x | | | | | | x | | | | | | x | | x | | x | |
| 1589 | | | | | x | x | | | | | | x | | | | | | x | | x | | | x |
| 1590 | | | | | x | x | | | | | | x | | | | | | x | | | x | x | |
| 1591 | | | | | x | x | | | | | | x | | | | | | x | | | x | | x |
| 1592 | | | | | x | x | | | | | | | x | x | | | | | x | | | x | |
| 1593 | | | | | x | x | | | | | | | x | x | | | | | x | | | | x |
| 1594 | | | | | x | x | | | | | | | x | x | | | | | | x | | x | |
| 1595 | | | | | x | x | | | | | | | x | x | | | | | | x | | | x |
| 1596 | | | | | x | x | | | | | | | x | x | | | | | | | x | x | |
| 1597 | | | | | x | x | | | | | | | x | x | | | | | | | x | | x |
| 1598 | | | | | x | x | | | | | | | x | | x | | | | x | | | x | |
| 1599 | | | | | x | x | | | | | | | x | | x | | | | x | | | | x |
| 1600 | | | | | x | x | | | | | | | x | | x | | | | | x | | x | |
| 1601 | | | | | x | x | | | | | | | x | | x | | | | | x | | | x |
| 1602 | | | | | x | x | | | | | | | x | | x | | | | | | x | x | |
| 1603 | | | | | x | x | | | | | | | x | | x | | | | | | x | | x |
| 1604 | | | | | x | x | | | | | | | x | | | x | | | x | | | x | |
| 1605 | | | | | x | x | | | | | | | x | | | x | | | x | | | | x |
| 1606 | | | | | x | x | | | | | | | x | | | x | | | | x | | x | |
| 1607 | | | | | x | x | | | | | | | x | | | x | | | | x | | | x |
| 1608 | | | | | x | x | | | | | | | x | | | x | | | | | x | x | |
| 1609 | | | | | x | x | | | | | | | x | | | x | | | | | x | | x |
| 1610 | | | | | x | x | | | | | | | x | | | | x | | x | | | x | |
| 1611 | | | | | x | x | | | | | | | x | | | | x | | x | | | | x |
| 1612 | | | | | x | x | | | | | | | x | | | | x | | | x | | x | |
| 1613 | | | | | x | x | | | | | | | x | | | | x | | | x | | | x |
| 1614 | | | | | x | x | | | | | | | x | | | | x | | | | x | x | |
| 1615 | | | | | x | x | | | | | | | x | | | | x | | | | x | | x |
| 1616 | | | | | x | x | | | | | | | x | | | | | x | x | | | x | |
| 1617 | | | | | x | x | | | | | | | x | | | | | x | x | | | | x |
| 1618 | | | | | x | x | | | | | | | x | | | | | x | | x | | x | |
| 1619 | | | | | x | x | | | | | | | x | | | | | x | | x | | | x |
| 1620 | | | | | x | x | | | | | | | x | | | | | x | | | x | x | |
| 1621 | | | | | x | x | | | | | | | x | | | | | x | | | x | | x |
| 1622 | | | | | x | | x | x | | | | | | x | | | | | x | | | x | |
| 1623 | | | | | x | | x | x | | | | | | x | | | | | x | | | | x |
| 1624 | | | | | x | | x | x | | | | | | x | | | | | | x | | x | |
| 1625 | | | | | x | | x | x | | | | | | x | | | | | | x | | | x |
| 1626 | | | | | x | | x | x | | | | | | x | | | | | | | x | x | |
| 1627 | | | | | x | | x | x | | | | | | x | | | | | | | x | | x |
| 1628 | | | | | x | | x | x | | | | | | | x | | | | x | | | x | |
| 1629 | | | | | x | | x | x | | | | | | | x | | | | x | | | | x |
| 1630 | | | | | x | | x | x | | | | | | | x | | | | | x | | x | |
| 1631 | | | | | x | | x | x | | | | | | | x | | | | | x | | | x |
| 1632 | | | | | x | | x | x | | | | | | | x | | | | | | x | x | |
| 1633 | | | | | x | | x | x | | | | | | | x | | | | | | x | | x |
| 1634 | | | | | x | | x | x | | | | | | | | x | | | x | | | x | |
| 1635 | | | | | x | | x | x | | | | | | | | x | | | x | | | | x |
| 1636 | | | | | x | | x | x | | | | | | | | x | | | | x | | x | |
| 1637 | | | | | x | | x | x | | | | | | | | x | | | | x | | | x |
| 1638 | | | | | x | | x | x | | | | | | | | x | | | | | x | x | |
| 1639 | | | | | x | | x | x | | | | | | | | x | | | | | x | | x |
| 1640 | | | | | x | | x | x | | | | | | | | | x | | x | | | x | |
| 1641 | | | | | x | | x | x | | | | | | | | | x | | x | | | | x |
| 1642 | | | | | x | | x | x | | | | | | | | | x | | | x | | x | |
| 1643 | | | | | x | | x | x | | | | | | | | | x | | | x | | | x |
| 1644 | | | | | x | | x | x | | | | | | | | | x | | | | x | x | |
| 1645 | | | | | x | | x | x | | | | | | | | | x | | | | x | | x |
| 1646 | | | | | x | | x | x | | | | | | | | | | x | x | | | x | |
| 1647 | | | | | x | | x | x | | | | | | | | | | x | x | | | | x |
| 1648 | | | | | x | | x | x | | | | | | | | | | x | | x | | x | |
| 1649 | | | | | x | | x | x | | | | | | | | | | x | | x | | | x |
| 1650 | | | | | x | | x | x | | | | | | | | | | x | | | x | x | |
| 1651 | | | | | x | | x | x | | | | | | | | | | x | | | x | | x |
| 1652 | | | | | x | | x | | x | | | | | x | | | | | x | | | x | |
| 1653 | | | | | x | | x | | x | | | | | x | | | | | x | | | | x |
| 1654 | | | | | x | | x | | x | | | | | x | | | | | | x | | x | |
| 1655 | | | | | x | | x | | x | | | | | x | | | | | | x | | | x |
| 1656 | | | | | x | | x | | x | | | | | x | | | | | | | x | x | |
| 1657 | | | | | x | | x | | x | | | | | x | | | | | | | x | | x |
| 1658 | | | | | x | | x | | x | | | | | | x | | | | x | | | x | |
| 1659 | | | | | x | | x | | x | | | | | | x | | | | x | | | | x |
| 1660 | | | | | x | | x | | x | | | | | | x | | | | | x | | x | |
| 1661 | | | | | x | | x | | x | | | | | | x | | | | | x | | | x |
| 1662 | | | | | x | | x | | x | | | | | | x | | | | | | x | x | |
| 1663 | | | | | x | | x | | x | | | | | | x | | | | | | x | | x |
| 1664 | | | | | x | | x | | x | | | | | | | x | | | x | | | x | |
| 1665 | | | | | x | | x | | x | | | | | | | x | | | x | | | | x |
| 1666 | | | | | x | | x | | x | | | | | | | x | | | | x | | x | |
| 1667 | | | | | x | | x | | x | | | | | | | x | | | | x | | | x |
| 1668 | | | | | x | | x | | x | | | | | | | x | | | | | x | x | |
| 1669 | | | | | x | | x | | x | | | | | | | x | | | | | x | | x |
| 1670 | | | | | x | | x | | x | | | | | | | | x | | x | | | x | |
| 1671 | | | | | x | | x | | x | | | | | | | | x | | x | | | | x |
| 1672 | | | | | x | | x | | x | | | | | | | | x | | | x | | x | |
| 1673 | | | | | x | | x | | x | | | | | | | | x | | | x | | | x |
| 1674 | | | | | x | | x | | x | | | | | | | | x | | | | x | x | |
| 1675 | | | | | x | | x | | x | | | | | | | | x | | | | x | | x |
| 1676 | | | | | x | | x | | x | | | | | | | | | x | x | | | x | |
| 1677 | | | | | x | | x | | x | | | | | | | | | x | x | | | | x |
| 1678 | | | | | x | | x | | x | | | | | | | | | x | | x | | x | |
| 1679 | | | | | x | | x | | x | | | | | | | | | x | | x | | | x |
| 1680 | | | | | x | | x | | x | | | | | | | | | x | | | x | x | |
| 1681 | | | | | x | | x | | x | | | | | | | | | x | | | x | | x |
| 1682 | | | | | x | | x | | | x | | | | x | | | | | x | | | x | |
| 1683 | | | | | x | | x | | | x | | | | x | | | | | x | | | | x |
| 1684 | | | | | x | | x | | | x | | | | x | | | | | | x | | x | |
| 1685 | | | | | x | | x | | | x | | | | x | | | | | | x | | | x |
| 1686 | | | | | x | | x | | | x | | | | x | | | | | | | x | x | |
| 1687 | | | | | x | | x | | | x | | | | x | | | | | | | x | | x |
| 1688 | | | | | x | | x | | | x | | | | | x | | | | x | | | x | |
| 1689 | | | | | x | | x | | | x | | | | | x | | | | x | | | | x |
| 1690 | | | | | x | | x | | | x | | | | | x | | | | | x | | x | |
| 1691 | | | | | x | | x | | | x | | | | | x | | | | | x | | | x |
| 1692 | | | | | x | | x | | | x | | | | | x | | | | | | x | x | |
| 1693 | | | | | x | | x | | | x | | | | | x | | | | | | x | | x |
| 1694 | | | | | x | | x | | | x | | | | | | x | | | x | | | x | |
| 1695 | | | | | x | | x | | | x | | | | | | x | | | x | | | | x |
| 1696 | | | | | x | | x | | | x | | | | | | x | | | | x | | x | |
| 1697 | | | | | x | | x | | | x | | | | | | x | | | | x | | | x |
| 1698 | | | | | x | | x | | | x | | | | | | x | | | | | x | x | |
| 1699 | | | | | x | | x | | | x | | | | | | x | | | | | x | | x |
| 1700 | | | | | x | | x | | | x | | | | | | | x | | x | | | x | |
| 1701 | | | | | x | | x | | | x | | | | | | | x | | x | | | | x |
| 1702 | | | | | x | | x | | | x | | | | | | | x | | | x | | x | |
| 1703 | | | | | x | | x | | | x | | | | | | | x | | | x | | | x |
| 1704 | | | | | x | | x | | | x | | | | | | | x | | | | x | x | |
| 1705 | | | | | x | | x | | | x | | | | | | | x | | | | x | | x |
| 1706 | | | | | x | | x | | | x | | | | | | | | x | x | | | x | |
| 1707 | | | | | x | | x | | | x | | | | | | | | x | x | | | | x |
| 1708 | | | | | x | | x | | | x | | | | | | | | x | | x | | x | |
| 1709 | | | | | x | | x | | | x | | | | | | | | x | | x | | | x |
| 1710 | | | | | x | | x | | | x | | | | | | | | x | | | x | x | |
| 1711 | | | | | x | | x | | | x | | | | | | | | x | | | x | | x |
| 1712 | | | | | x | | x | | | | x | | | x | | | | | x | | | x | |
| 1713 | | | | | x | | x | | | | x | | | x | | | | | x | | | | x |
| 1714 | | | | | x | | x | | | | x | | | x | | | | | | x | | x | |
| 1715 | | | | | x | | x | | | | x | | | x | | | | | | x | | | x |
| 1716 | | | | | x | | x | | | | x | | | x | | | | | | | x | x | |
| 1717 | | | | | x | | x | | | | x | | | x | | | | | | | x | | x |
| 1718 | | | | | x | | x | | | | x | | | | x | | | | x | | | x | |
| 1719 | | | | | x | | x | | | | x | | | | x | | | | x | | | | x |
| 1720 | | | | | x | | x | | | | x | | | | x | | | | | x | | x | |
| 1721 | | | | | x | | x | | | | x | | | | x | | | | | x | | | x |
| 1722 | | | | | x | | x | | | | x | | | | x | | | | | | x | x | |
| 1723 | | | | | x | | x | | | | x | | | | x | | | | | | x | | x |
| 1724 | | | | | x | | x | | | | x | | | | | x | | | x | | | x | |
| 1725 | | | | | x | | x | | | | x | | | | | x | | | x | | | | x |
| 1726 | | | | | x | | x | | | | x | | | | | x | | | | x | | x | |
| 1727 | | | | | x | | x | | | | x | | | | | x | | | | x | | | x |
| 1728 | | | | | x | | x | | | | x | | | | | x | | | | | x | x | |
| 1729 | | | | | x | | x | | | | x | | | | | x | | | | | x | | x |
| 1730 | | | | | x | | x | | | | x | | | | | | x | | x | | | x | |
| 1731 | | | | | x | | x | | | | x | | | | | | x | | x | | | | x |
| 1732 | | | | | x | | x | | | | x | | | | | | x | | | x | | x | |
| 1733 | | | | | x | | x | | | | x | | | | | | x | | | x | | | x |
| 1734 | | | | | x | | x | | | | x | | | | | | x | | | | x | x | |
| 1735 | | | | | x | | x | | | | x | | | | | | x | | | | x | | x |
| 1736 | | | | | x | | x | | | | x | | | | | | | x | x | | | x | |
| 1737 | | | | | x | | x | | | | x | | | | | | | x | x | | | | x |
| 1738 | | | | | x | | x | | | | x | | | | | | | x | | x | | x | |
| 1739 | | | | | x | | x | | | | x | | | | | | | x | | x | | | x |
| 1740 | | | | | x | | x | | | | x | | | | | | | x | | | x | x | |
| 1741 | | | | | x | | x | | | | x | | | | | | | x | | | x | | x |
| 1742 | | | | | x | | x | | | | | x | | x | | | | | x | | | x | |
| 1743 | | | | | x | | x | | | | | x | | x | | | | | x | | | | x |
| 1744 | | | | | x | | x | | | | | x | | x | | | | | | x | | x | |
| 1745 | | | | | x | | x | | | | | x | | x | | | | | | x | | | x |
| 1746 | | | | | x | | x | | | | | x | | x | | | | | | | x | x | |
| 1747 | | | | | x | | x | | | | | x | | x | | | | | | | x | | x |
| 1748 | | | | | x | | x | | | | | x | | | x | | | | x | | | x | |
| 1749 | | | | | x | | x | | | | | x | | | x | | | | x | | | | x |
| 1750 | | | | | x | | x | | | | | x | | | x | | | | | x | | x | |
| 1751 | | | | | x | | x | | | | | x | | | x | | | | | x | | | x |
| 1752 | | | | | x | | x | | | | | x | | | x | | | | | | x | x | |
| 1753 | | | | | x | | x | | | | | x | | | x | | | | | | x | | x |
| 1754 | | | | | x | | x | | | | | x | | | | x | | | x | | | x | |
| 1755 | | | | | x | | x | | | | | x | | | | x | | | x | | | | x |
| 1756 | | | | | x | | x | | | | | x | | | | x | | | | x | | x | |
| 1757 | | | | | x | | x | | | | | x | | | | x | | | | x | | | x |
| 1758 | | | | | x | | x | | | | | x | | | | x | | | | | x | x | |
| 1759 | | | | | x | | x | | | | | x | | | | x | | | | | x | | x |
| 1760 | | | | | x | | x | | | | | x | | | | | x | | x | | | x | |
| 1761 | | | | | x | | x | | | | | x | | | | | x | | x | | | | x |
| 1762 | | | | | x | | x | | | | | x | | | | | x | | | x | | x | |
| 1763 | | | | | x | | x | | | | | x | | | | | x | | | x | | | x |
| 1764 | | | | | x | | x | | | | | x | | | | | x | | | | x | x | |
| 1765 | | | | | x | | x | | | | | x | | | | | x | | | | x | | x |
| 1766 | | | | | x | | x | | | | | x | | | | | | x | x | | | x | |
| 1767 | | | | | x | | x | | | | | x | | | | | | x | x | | | | x |
| 1768 | | | | | x | | x | | | | | x | | | | | | x | | x | | x | |
| 1769 | | | | | x | | x | | | | | x | | | | | | x | | x | | | x |
| 1770 | | | | | x | | x | | | | | x | | | | | | x | | | x | x | |
| 1771 | | | | | x | | x | | | | | x | | | | | | x | | | x | | x |
| 1772 | | | | | x | | x | | | | | | x | x | | | | | x | | | x | |
| 1773 | | | | | x | | x | | | | | | x | x | | | | | x | | | | x |
| 1774 | | | | | x | | x | | | | | | x | x | | | | | | x | | x | |
| 1775 | | | | | x | | x | | | | | | x | x | | | | | | x | | | x |
| 1776 | | | | | x | | x | | | | | | x | x | | | | | | | x | x | |
| 1777 | | | | | x | | x | | | | | | x | x | | | | | | | x | | x |
| 1778 | | | | | x | | x | | | | | | x | | x | | | | x | | | x | |
| 1779 | | | | | x | | x | | | | | | x | | x | | | | x | | | | x |
| 1780 | | | | | x | | x | | | | | | x | | x | | | | | x | | x | |
| 1781 | | | | | x | | x | | | | | | x | | x | | | | | x | | | x |
| 1782 | | | | | x | | x | | | | | | x | | x | | | | | | x | x | |
| 1783 | | | | | x | | x | | | | | | x | | x | | | | | | x | | x |
| 1784 | | | | | x | | x | | | | | | x | | | x | | | x | | | x | |
| 1785 | | | | | x | | x | | | | | | x | | | x | | | x | | | | x |
| 1786 | | | | | x | | x | | | | | | x | | | x | | | | x | | x | |
| 1787 | | | | | x | | x | | | | | | x | | | x | | | | x | | | x |
| 1788 | | | | | x | | x | | | | | | x | | | x | | | | | x | x | |
| 1789 | | | | | x | | x | | | | | | x | | | x | | | | | x | | x |
| 1790 | | | | | x | | x | | | | | | x | | | | x | | x | | | x | |
| 1791 | | | | | x | | x | | | | | | x | | | | x | | x | | | | x |
| 1792 | | | | | x | | x | | | | | | x | | | | x | | | x | | x | |
| 1793 | | | | | x | | x | | | | | | x | | | | x | | | x | | | x |
| 1794 | | | | | x | | x | | | | | | x | | | | x | | | | x | x | |
| 1795 | | | | | x | | x | | | | | | x | | | | x | | | | x | | x |
| 1796 | | | | | x | | x | | | | | | x | | | | | x | x | | | x | |
| 1797 | | | | | x | | x | | | | | | x | | | | | x | x | | | | x |
| 1798 | | | | | x | | x | | | | | | x | | | | | x | | x | | x | |
| 1799 | | | | | x | | x | | | | | | x | | | | | x | | x | | | x |
| 1800 | | | | | x | | x | | | | | | x | | | | | x | | | x | x | |
| 1801 | | | | | x | | x | | | | | | x | | | | | x | | | x | | x |

### V. EXAMPLES

### Example 1

### Synthesis of Compound 2-F

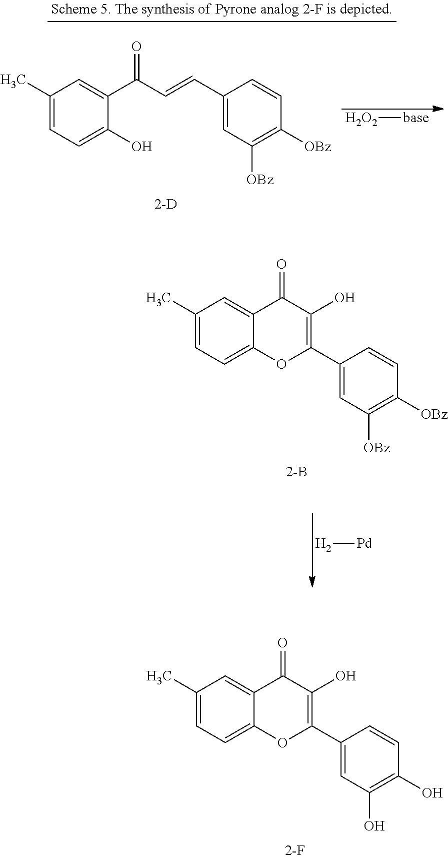

The synthesis of compound 2-F is shown in Scheme 5. Vinylogous ketone 2-D is cyclized in the presence of hydrogen peroxide and base to form the dibenzyl protected flavonoid 2-E. Hydrogenolysis with hydrogen with palladium catalysis deprotects the benzyl ethers to yield the 3′,4′-dihydroxy phenyl pyrone analog 2-F.

### Example 2

### Synthesis of Compound 3-F

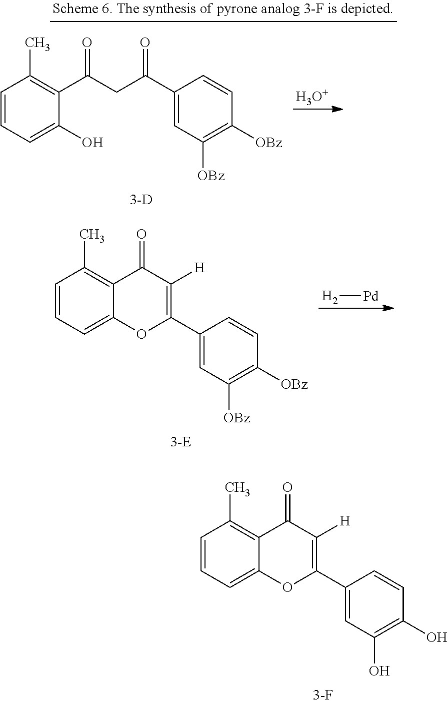

The synthesis of pyrone analog 3-F is accomplished via synthetic steps as shown in Scheme 6. Propanedione 3-D is cyclized under acidic conditions to yield the 3-H dibenzyloxy flavone analog. Deprotection of the benzyl ethers via hydrogenolysis produces pyrone analog 3-F.

### Example 3

### Synthesis of Compound 4-J

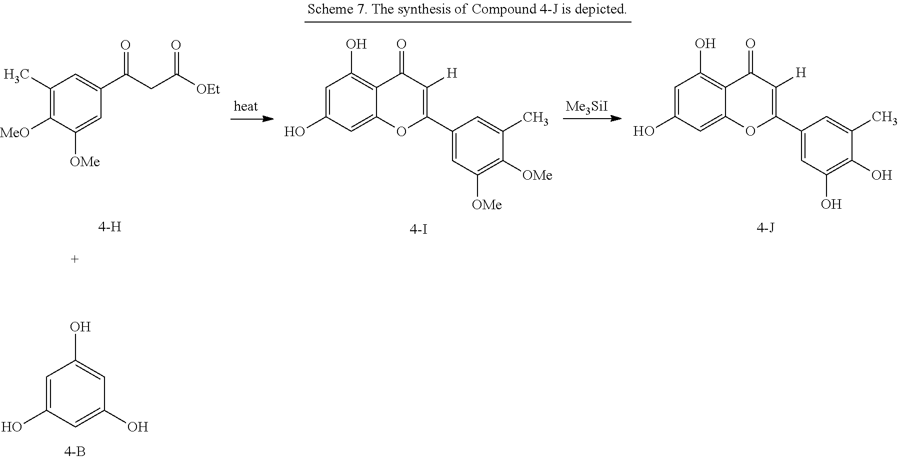

The trihydroxy benzene 4-B is condensed with propanedione 4-H to obtain the 3′,4′-dimethoxy phenyl flavone 4-I. Deprotection of the methyl ethers at the 3′ and 4′ positions with trimethylsilyl iodide produces pyrone analog 4-J.

### Example 4

### Reversal Effect of Modulator, Pyrone Analog (PA), on Sedative Effects in Rodents

An anesthetic wake up test is used to assess the reversal effect of modulator, PA, on the sedative effects of barbiturates, opioids, and benzodiazepines. This is a single blind, randomized, controlled animal trial. Approximately 48 rodents may be utilized throughout the study. Animals may be reused. However, a washout of 24 hours is required between exposures.

Twelve rodents can be utilized in each portion of this trial. Intravenous barbiturate (e.g. diprivan, pentobarbital, or phenobarbital) anesthesia is induced and titrated to spontaneous but slow respirations and lack of response to painful stimulation. Supplemental oxygen may be delivered. A maximum of 3 doses of intraperitoneal PA may be tested (low, medium, high) along with placebo. Once administered rodents are monitored with the help of stopwatch for time to awakening and return to normal respiratory rate. Once awakened, rodents are tested for criteria including time to withdrawal from painful stimulus and performance on rotarod.

This study may be repeated as a single agent trial with opioids (remifentanyl, fentanyl, morphine, etc) and benzodiazepines (diazepam, midazolam, lorazepam). This study can also be repeated as a multi agent trial utilizing one opioid, one benzodiazepine, and one barbiturate.

### Example 5

### Pyrone Analog (PgP Modulator) Ameliorates CNS Effects of Oxycodone in an Acute Pain Model

Opioids frequently produce adverse CNS side effects in ambulatory settings, providing a rationale for improving opioid analgesia by minimizing adverse effects. Pyrone analogs may activate PgP efflux of pump ligands at the blood brain barrier. In this pilot study, the hypothesis that pyrone analogs can improve the tolerability and safety of oxycodone without impairing analgesia is tested. Healthy subjects (up to approximately 65 individuals) undergoing third molar removal are randomly allocated to receive, for example, 500 mg oral PA or matching placebo at 1 hr prior to surgery. All subjects receive, for example, 10 mg oral oxycodone immediately prior to surgery. Oxycodone concentrations do not differ between groups prior to surgery, at 1 hr, or at 4 hr. Total Nausea and Vomiting Score (TNVS) is calculated for the time from dosing through 24 hr.

All of the methods disclosed and claimed herein can be made and executed without undue experimentation in light of the present disclosure. It will be apparent to those of skill in the art that variations may be applied without departing from the concept, spirit and scope of the invention. More specifically, it will be apparent that certain agents that both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

1. A compound of Formula I: 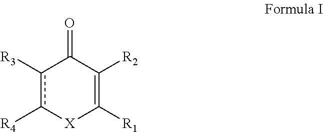
and its pharmaceutically acceptable salts or esters,
wherein X is O, S, or NR′, □wherein R′ is hydrogen, C₁-C₁₀ alkyl, C₂-C₁₀ alkynyl, C₂-C₁₀ alkenyl, C₁-C₁₀ aliphatic acyl, C₆-C₁₀ aromatic acyl, C₆-C₁₀ aralkyl acyl, C₆-C₁₀alkylaryl acyl, aryl, C₃-C₁₀heterocyclyl, heteroaryl, or C₃-C₁₀cycloalkyl;
R₁, and R₂ are independently hydrogen, hydroxyl, C₁-C₁₀ alkyl, C₂-C₁₀ alkynyl, C₂-C₁₀ alkenyl, carboxyl, carbohydrate, ester, acyloxy, nitro, halogen, C₁-C₁₀ aliphatic acyl, C₆-C₁₀ aromatic acyl, C₆-C₁₀ aralkyl acyl, C₆-C₁₀ alkylaryl acyl, alkoxy, amine, aryl, C₄-C₁₀heterocyclyl, heteroaryl, C₃-C₁₀cycloalkyl, —OPO₃WY, —OCH₂PO₄WY, —OCH₂PO₄Z or —OPO₃Z;
R₃ and R₄ are independently hydrogen, hydroxyl, C₁-C₁₀ alkyl, C₂-C₁₀ alkynyl, C₂-C₁₀ alkenyl, carboxyl, Carbohydrate, ester, acyloxy, nitro, halogen, C₁-C₁₀ aliphatic acyl, C₆-C₁₀ aromatic acyl C₆-C₁₀ aralkyl acyl, C₆-C₁₀ alkylaryl acyl, alkoxy, amine, aryl, C₄-C₁₀heterocyclyl, heteroaryl, C₃-C₁₀cycloalkyl, —OPO₃WY, —OCH₂PO₄WY, —OCH₂PO₄Z or —OPO₃Z;
or R₃ and R₄ are taken together to form a C₅-C₁₀heterocyclyl, C₅-C₁₀cycloalkyl, aryl, or heteroaryl;
W and Y are independently hydrogen, methyl, ethyl, alkyl, carbohydrate, or a cation, and Z is a multivalent cation; and
wherein the compound of Formula I is not the compound of Formula A: 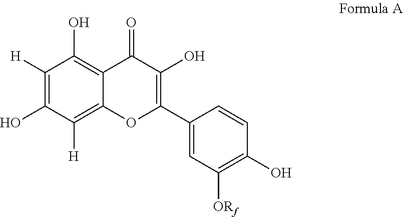
wherein R_{f} is hydrogen; and
wherein the compound of Formula I is not one of the following compounds:

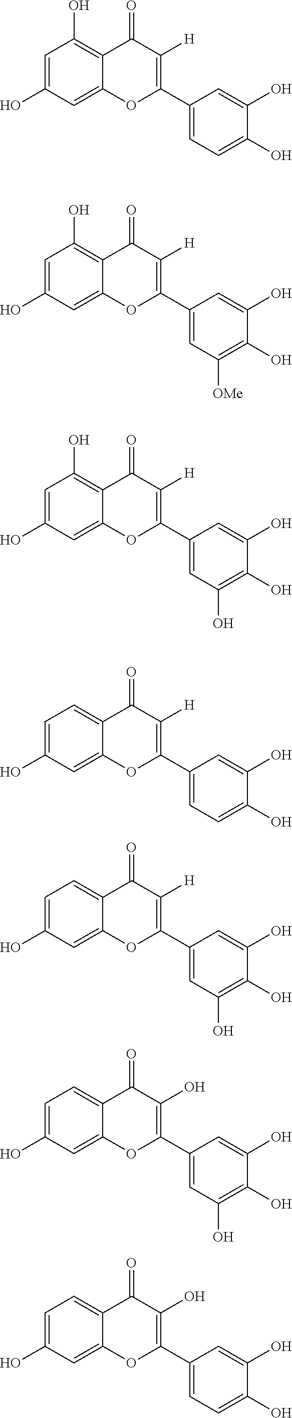

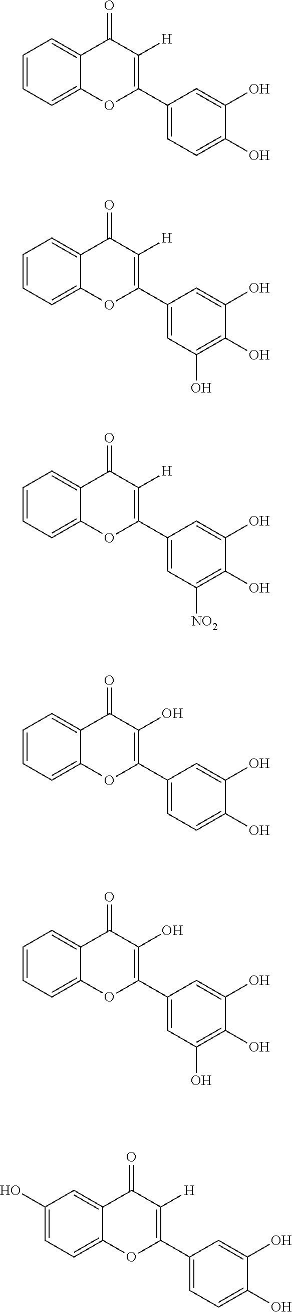

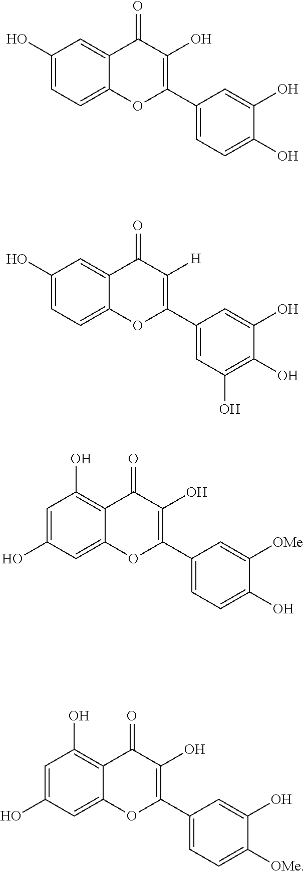

2. The compound of claim 1 wherein the compound is of Formula II: 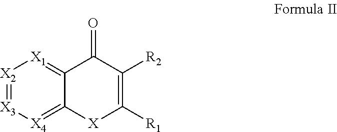
wherein X₁, X₂, X₃, and X₄ are independently CR₅, O, S, or N; and
each instance of R₅ is independently hydrogen, hydroxyl, carboxaldehyde, amino, C₁-C₁₀ alkyl, C₂-C₁₀ alkynyl, C₂-C₁₀ alkenyl, carboxyl, Carbohydrate, ester, acyloxy, nitro, halogen, C₁-C₁₀ aliphatic acyl, C₆-C₁₀ aromatic acyl, C₆-C₁₀ aralkyl acyl, C₆-C₁₀ alkylaryl acyl, alkoxy, amine, aryl, C₃-C₁₀ heterocyclyl, heteroaryl, C₃-C₁₀cycloalkyl, —OPO₃WY, —OCH₂PO₄WY, —OCH₂PO₄Z or —OPO₃Z.

3-5. (canceled)

6. A compound of Formula VII: 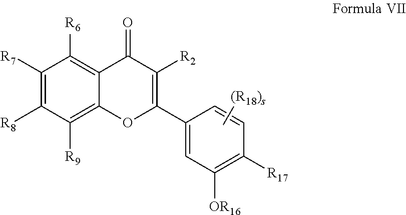
and its pharmaceutically acceptable salts thereof, wherein
R₁, and R₂ are independently hydrogen, hydroxyl, C₁-C₁₀ alkyl, C₂-C₁₀ alkynyl, C₂-C₁₀ alkenyl, carboxyl, carbohydrate, ester, acyloxy, nitro, halogen, C₁-C₁₀ aliphatic acyl, C₆-C₁₀ aromatic acyl, C₆-C₁₀ aralkyl acyl, C₆-C₁₀ alkylaryl acyl, alkoxy, amine, aryl, C₄-C₁₀heterocyclyl, heteroaryl, C₃-C₁₀cycloalkyl, —OPO₃WY, —OCH₂PO₄WY, —OCH₂PO₄Z or —OPO₃Z;
R₆, R₇, R₈, and R₉ are independently hydrogen, hydroxyl, carboxaldehyde, amino, C₁-C₁₀ alkyl, C₂-C₁₀ alkynyl, C₂-C₁₀ alkenyl, carboxyl, carbohydrate, ester, acyloxy, nitro, halogen, C₁-C₁₀ aliphatic acyl, C₆-C₁₀ aromatic acyl, C₆-C₁₀ aralkyl acyl, C₆-C₁₀ alkylaryl acyl, alkoxy, amine, aryl, C₃-C₁₀ heterocyclyl, heteroaryl, C₃-C₁₀cycloalkyl, —OPO₃WY, —OCH₂PO₄WY, —OCH₂PO₄Z or —OPO₃Z;
R₁₆ is hydrogen, C₁-C₁₀ alkyl, C₂-C₁₀ alkynyl, C₂-C₁₀ alkenyl, carbohydrate C₁-C₁₀ aliphatic acyl, C₆-C₁₀ aromatic acyl, C₆-C₁₀ aralkyl acyl, C₆-C₁₀alkylaryl acyl, aryl, C₃-C₁₀heterocyclyl, heteroaryl, C₃-C₁₀cycloalkyl, —PO₃WY, —CH₂PO₄WY, —CH₂PO₄Z or —PO₃Z;
R₁₇ is hydrogen, hydroxy, carboxaldehyde, amine, C₁-C₁₀ alkyl, C₂-C₁₀ alkynyl, C₂-C₁₀ alkenyl, carboxyl, carbohydrate, ester, acyloxy, nitro, halogen, C₁-C₁₀ aliphatic acyl, C₆-C₁₀ aromatic acyl, C₆-C₁₀ aralkyl acyl, C₆-C₁₀ alkylaryl acyl, alkoxy, aryl, C₃-C₁₀ heterocyclyl, heteroaryl, or C₃-C₁₀cycloalkyl, —OPO₃WY, —OCH₂PO₄WY, —OCH₂PO₄Z or —OPO₃Z;
each instance of R₁₈ is independently hydrogen, hydroxyl, carboxaldehyde, amine, C₁-C₁₀ alkyl, C₂-C₁₀ alkynyl, C₂-C₁₀ alkenyl, carboxyl, carbohydrate, ester, acyloxy, nitro, halogen, C₁-C₁₀ aliphatic acyl, C₆-C₁₀ aromatic acyl, C₆-C₁₀ aralkyl acyl, C₆-C₁₀ alkylaryl acyl, alkoxy, alkyl, phosphate, aryl, heteroaryl, C₃-C₁₀ heterocyclic, C₃-C₁₀cycloalkyl, —OPO₃WY, —OCH₂PO₄WY, —OCH₂PO₄Z or —OPO₃Z;
s is an integer of 0, 1, 2, or 3; and
wherein the compound of Formula I is not the compound of Formula A: 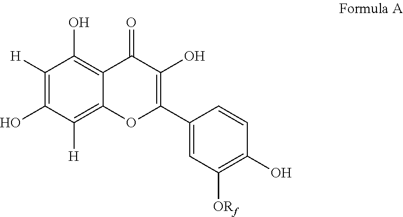
wherein R_{f} is hydrogen; and
wherein the compound of Formula VII is not the compound wherein:
R₂ is hydrogen, R₆, R₇, R₈, and R₉ are hydrogen, R₁₆ is hydrogen, R₁₇ is hydroxy, and s is 0;
R₂ is hydrogen, R₆, R₇, R₈, and R₉ are hydrogen, R₁₆ is hydrogen, R₁₇ is hydroxy, s is 1, and R₁₈ is 5′-hydroxy;
R₂ is hydrogen, R₆, R₇, R₈, and R₉ are hydrogen, R₁₆ is hydrogen, R₁₇ is hydroxy, s is 1, and R₁₈ is 5′-nitro;
R₂ is hydroxy, R₆, R₇, R₈, and R₉ are hydrogen, R₁₆ is hydrogen, R₁₇ is hydroxy, and s is 0;
R₂ is hydroxy, R₆, R₇, R₈, and R₉ are hydrogen, R₁₆ is hydrogen, R₁₇ is hydroxy, s is 1, and R₁₈ is 5′-hydroxy;
R₂ is hydrogen, R₆ and R₈ are hydroxy, R₇ and R₉ are hydrogen, R₁₆ is hydrogen, R₁₇ is hydroxy, and s is 0;
R₂ is hydrogen, R₆ and R₈ are hydroxy, R₇ and R₉ are hydrogen, R₁₆ is hydrogen, R₁₇ is hydroxy, s is 1, and R₁₈ is 5′-hydroxy;
R₂ is hydrogen, R₆ and R₈ are hydroxy, R₇ and R₉ are hydrogen, R₁₆ is hydrogen, R₁₇ is hydroxy, s is 1, and R₁₈ is 5′-methoxy;
R₂ is hydroxy, R₆ and R₈ are hydroxy, R₇ and R₉ are hydrogen, R₁₆ is methyl, R₁₇ is hydroxy, and s is 0;
R₂ is hydroxy, R₆ and R₈ are hydroxy, R₇ and R₉ are hydrogen, R₁₆ is hydrogen, R₁₇ is methoxy, and s is 0;
R₂ is hydrogen, R₆, R₈, and R₉ are hydrogen, R₇ is hydroxy, R₁₆ is hydrogen, R₁₇ is hydroxy, and s is 0;
R₂ is hydrogen, R₆, R₈, and R₉ are hydrogen, R₇ is hydroxy, R₁₆ is hydrogen, R₁₇ is hydroxy, s is 1, and R₁₈ is 5′-hydroxy;
R₂ is hydroxy, R₆, R₈, and R₉ are hydrogen, R₇ is hydroxy, R₁₆ is hydrogen, R₁₇ is hydroxy, and s is 0;
R₂ is hydrogen, R₆, R₇, and R₉ are hydrogen, R₈ is hydroxy, R₁₆ is hydrogen, R₁₇ is hydroxy, and s is 0;
R₂ is hydrogen, R₆, R₇, and R₉ are hydrogen, R₈ is hydroxy, R₁₆ is hydrogen, R₁₇ is hydroxy, s is 1, and R₁₈ is 5′-hydroxy;
R₂ is hydroxy, R₆, R₇, and R₉ are hydrogen, R₈ is hydroxy, R₁₆ is hydrogen, R₁₇ is hydroxy, and s is 0; or
R₂ is hydroxy, R₆, R₇, and R₉ are hydrogen, R₈ is hydroxy, R₁₆ is hydrogen, R₁₇ is hydroxy, s is 1, and R₁₈ is 5′-hydroxy.

7. (canceled)

8. The compound of claim 6 of Formula VIII: 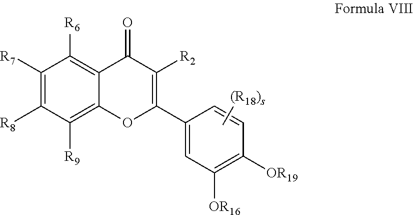
wherein R₁₉ is hydrogen, C₁-C₁₀ alkyl, C₂-C₁₀ alkynyl, C₂-C₁₀ alkenyl, carbohydrate, C₁-C₁₀ aliphatic acyl, C₆-C₁₀ aromatic acyl, C₆-C₁₀ aralkyl acyl, C₆-C₁₀ alkylaryl acyl, aryl, C₃-C₁₀ heterocyclyl, heteroaryl, optionally substituted C₃-C₁₀cycloalkyl, —PO₃WY, —CH₂PO₄WY, —CH₂PO₄Z or —PO₃Z; and
at least one of R₁₆ and R₁₉ is —PO₃WY, —CH₂PO₄WY, —CH₂PO₄Z or —PO₃Z.

9. The compound of claim 6 of Formula IX: 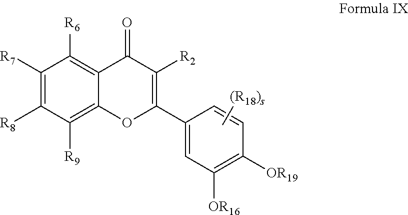
wherein R₆, R₇, R₈, and R₉ are independently hydrogen, carboxaldehyde, amino, C₁-C₁₀ alkyl, C₂-C₁₀ alkynyl, C₂-C₁₀ alkenyl, carboxyl, carbohydrate, ester, acyloxy, nitro, halogen, C₁-C₁₀ aliphatic acyl, C₆-C₁₀ aromatic acyl, C₆-C₁₀ aralkyl acyl, C₆-C₁₀ alkylaryl acyl, alkoxy, amine, aryl, C₃-C₁₀ heterocyclyl, heteroaryl, C₃-C₁₀cycloalkyl, —OPO₃WY, —OCH₂PO₄WY, —OCH₂PO₄Z or —OPO₃Z.

10-12. (canceled)

13. The compound of claim 1 of Formula XIII: 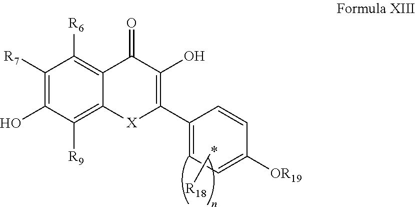
wherein R₆, R₇, and R₉ are independently hydrogen, hydroxyl, carboxaldehyde, amino, C₁-C₁₀ alkyl, C₂-C₁₀ alkynyl, C₂-C₁₀ alkenyl, carboxyl, carbohydrate, ester, acyloxy, nitro, halogen, C₁-C₁₀ aliphatic acyl, C₆-C₁₀ aromatic acyl, C₆-C₁₀ aralkyl acyl, C₆-C₁₀ alkylaryl acyl, alkoxy, amine, aryl, C₃-C₁₀ heterocyclyl, heteroaryl, C₃-C₁₀cycloalkyl, —OPO₃WY, —OCH₂PO₄WY, —OCH₂PO₄Z or —OPO₃Z;
each instance of R₁₈ is independently hydrogen, hydroxyl, carboxaldehyde, amine, C₁-C₁₀ alkyl, C₂-C₁₀ alkynyl, C₂-C₁₀ alkenyl, carboxyl, carbohydrate, ester, acyloxy, nitro, halogen, C₁-C₁₀ aliphatic acyl, C₆-C₁₀ aromatic acyl, C₆-C₁₀ aralkyl acyl, C₆-C₁₀ alkylaryl acyl, alkoxy, alkyl, phosphate, aryl, heteroaryl, C₃-C₁₀ heterocyclic, C₃-C₁₀cycloalkyl, —OPO₃WY, —OCH₂PO₄WY, —OCH₂PO₄Z or —OPO₃Z;
R₁₉ is hydrogen, C₁-C₁₀ alkyl, C₂-C₁₀ alkynyl, C₂-C₁₀ alkenyl, carbohydrate, C₁-C₁₀ aliphatic acyl, C₆-C₁₀ aromatic acyl, C₆-C₁₀ aralkyl acyl, C₆-C₁₀ alkylaryl acyl, aryl, C₃-C₁₀ heterocyclyl, heteroaryl, optionally substituted C₃-C₁₀cycloalkyl, —PO₃WY, —CH₂PO₄WY, —CH₂PO₄Z or —PO₃Z;
n is an integer of 0, 1, 2, 3, or 4.

14. The compound of claim 1 of Formula XV: 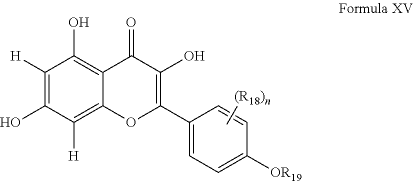
wherein each instance of R₁₈ is independently hydrogen, hydroxyl, carboxaldehyde, amine, C₁-C₁₀ alkyl, C₂-C₁₀ alkynyl, C₂-C₁₀ alkenyl, carboxyl, carbohydrate, ester, acyloxy, nitro, halogen, C₁-C₁₀ aliphatic acyl, C₆-C₁₀ aromatic acyl, C₆-C₁₀ aralkyl acyl, C₆-C₁₀ alkylaryl acyl, alkoxy, alkyl, phosphate, aryl, heteroaryl, C₃-C₁₀heterocyclic, C₃-C₁₀cycloalkyl, —OPO₃WY, —OCH₂PO₄WY, —OCH₂PO₄Z or —OPO₃Z;
R₁₉ is hydrogen, C₁-C₁₀ alkyl, C₂-C₁₀ alkynyl, C₂-C₁₀ alkenyl, carbohydrate, C₁-C₁₀ aliphatic acyl, C₆-C₁₀ aromatic acyl, C₆-C₁₀ aralkyl acyl, C₆-C₁₀ alkylaryl acyl, aryl, C₃-C₁₀ heterocyclyl, heteroaryl, optionally substituted C₃-C₁₀cycloalkyl, —PO₃WY, —CH₂PO₄WY, —CH₂PO₄Z or —PO₃Z; and
n is an integer of 0, 1, 2, 3, or 4.

15. The compound of claim 1 of Formula XVI: 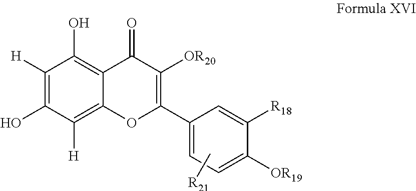
wherein R₁₈ and R₂₁ are independently hydrogen, hydroxyl, carboxaldehyde, amine, C₁-C₁₀ alkyl, C₂-C₁₀ alkynyl, C₂-C₁₀ alkenyl, carboxyl, carbohydrate, ester, acyloxy, nitro, halogen, C₁-C₁₀ aliphatic acyl, C₆-C₁₀ aromatic acyl, C₆-C₁₀ aralkyl acyl, C₆-C₁₀ alkylaryl acyl, alkoxy, alkyl, phosphate, aryl, heteroaryl, C₃-C₁₀ heterocyclic, C₃-C₁₀cycloalkyl, —OPO₃WY, —OCH₂PO₄WY, —OCH₂PO₄Z or —OPO₃Z;
R₁₉ and R₂₀ are independently hydrogen, C₁-C₁₀ alkyl, C₂-C₁₀ alkynyl, C₂-C₁₀ alkenyl, carbohydrate, C₁-C₁₀ aliphatic acyl, C₆-C₁₀ aromatic acyl, C₆-C₁₀ aralkyl acyl, C₆-C₁₀ alkylaryl acyl, aryl, C₃-C₁₀ heterocyclyl, heteroaryl, optionally substituted C₃-C₁₀cycloalkyl, —PO₃WY, —CH₂PO₄WY, —CH₂PO₄Z or —PO₃Z.

16. The compound of claim 1 of Formula XVII: 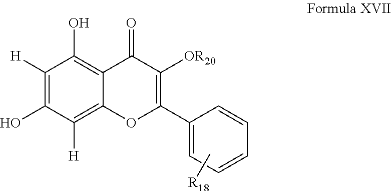
wherein R₁₈ is hydrogen, hydroxyl, carboxaldehyde, amine, C₁-C₁₀ alkyl, C₂-C₁₀ alkynyl, C₂-C₁₀ alkenyl, carboxyl, carbohydrate, ester, acyloxy, nitro, halogen, C₁-C₁₀ aliphatic acyl, C₆-C₁₀ aromatic acyl, C₆-C₁₀ aralkyl acyl, C₆-C₁₀ alkylaryl acyl, alkoxy, alkyl, phosphate, aryl, heteroaryl, C₃-C₁₀ heterocyclic, C₃-C₁₀cycloalkyl, —OPO₃WY, —OCH₂PO₄WY, —OCH₂PO₄Z or —OPO₃Z;
R₂₀ is hydrogen, C₁-C₁₀ alkyl, C₂-C₁₀ alkynyl, C₂-C₁₀ alkenyl, carbohydrate, C₁-C₁₀ aliphatic acyl, C₆-C₁₀ aromatic acyl, C₆-C₁₀ aralkyl acyl, C₆-C₁₀ alkylaryl acyl, aryl, C₃-C₁₀ heterocyclyl, heteroaryl, optionally substituted C₃-C₁₀cycloalkyl, —PO₃WY, —CH₂PO₄WY, —CH₂PO₄Z or —PO₃Z.

17. The compound of claim 1 of Formula XVIII: 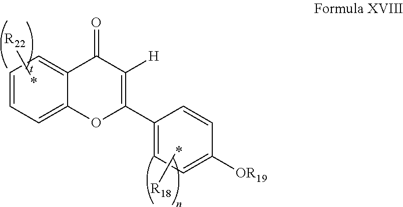
wherein each instance of R₁₈ and R₂₂ is independently hydrogen, hydroxyl, carboxaldehyde, amine, C₁-C₁₀ alkyl, C₂-C₁₀ alkynyl, C₂-C₁₀ alkenyl, carboxyl, carbohydrate, ester, acyloxy, nitro, halogen, C₁-C₁₀ aliphatic acyl, C₆-C₁₀ aromatic acyl, C₆-C₁₀ aralkyl acyl, C₆-C₁₀ alkylaryl acyl, alkoxy, alkyl, phosphate, aryl, heteroaryl, C₃-C₁₀heterocyclic, C₃-C₁₀cycloalkyl, —OPO₃WY, —OCH₂PO₄WY, —OCH₂PO₄Z or —OPO₃Z;
R₁₉ is hydrogen, C₁-C₁₀ alkyl, C₂-C₁₀ alkynyl, C₂-C₁₀ alkenyl, carbohydrate, C₁-C₁₀ aliphatic acyl, C₆-C₁₀ aromatic acyl, C₆-C₁₀ aralkyl acyl, C₆-C₁₀ alkylaryl acyl, aryl, C₃-C₁₀ heterocyclyl, heteroaryl, optionally substituted C₃-C₁₀cycloalkyl, —PO₃WY, —CH₂PO₄WY, —CH₂PO₄Z or —PO₃Z;
n is an integer of 0, 1, 2, 3, or 4; and
t is an integer of 0, 1, 2, 3, or 4.

18. The compound of claim 1 of Formula XIX: 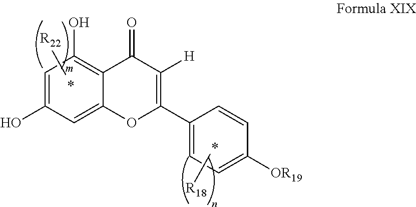
wherein each instance of R₁₈ and R₂₂ is independently hydrogen, hydroxyl, carboxaldehyde, amine, C₁-C₁₀ alkyl, C₂-C₁₀ alkynyl, C₂-C₁₀ alkenyl, carboxyl, carbohydrate, ester, acyloxy, nitro, halogen, C₁-C₁₀ aliphatic acyl, C₆-C₁₀ aromatic acyl, C₆-C₁₀ aralkyl acyl, C₆-C₁₀ alkylaryl acyl, alkoxy, alkyl, phosphate, aryl, heteroaryl, C₃-C₁₀heterocyclic, C₃-C₁₀cycloalkyl, —OPO₃WY, —OCH₂PO₄WY, —OCH₂PO₄Z or —OPO₃Z;
R₁₉ is hydrogen, C₁-C₁₀ alkyl, C₂-C₁₀ alkynyl, C₂-C₁₀ alkenyl, carbohydrate, C₁-C₁₀ aliphatic acyl, C₆-C₁₀ aromatic acyl, C₆-C₁₀ aralkyl acyl, C₆-C₁₀ alkylaryl acyl, aryl, C₃-C₁₀ heterocyclyl, heteroaryl, optionally substituted C₃-C₁₀cycloalkyl, —PO₃WY, —CH₂PO₄WY, —CH₂PO₄Z or —PO₃Z;
n is an integer of 0, 1, 2, 3, or 4; and
m is an integer of 0, 1, or 2.

19. The compound of claim 1 of Formula XX: 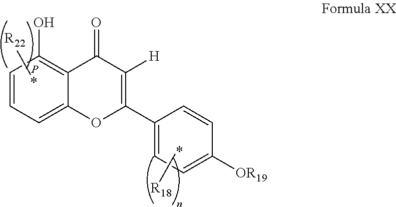
wherein each instance of R₁₈ and R₂₂ is independently hydrogen, hydroxyl, carboxaldehyde, amine, C₁-C₁₀ alkyl, C₂-C₁₀ alkynyl, C₂-C₁₀ alkenyl, carboxyl, carbohydrate, ester, acyloxy, nitro, halogen, C₁-C₁₀ aliphatic acyl, C₆-C₁₀ aromatic acyl, C₆-C₁₀ aralkyl acyl, C₆-C₁₀ alkylaryl acyl, alkoxy, alkyl, phosphate, aryl, heteroaryl, C₃-C₁₀heterocyclic, C₃-C₁₀cycloalkyl, —OPO₃WY, —OCH₂PO₄WY, —OCH₂PO₄Z or —OPO₃Z;
R₁₉ is hydrogen, C₁-C₁₀ alkyl, C₂-C₁₀ alkynyl, C₂-C₁₀ alkenyl, carbohydrate, C₁-C₁₀ aliphatic acyl, C₆-C₁₀ aromatic acyl, C₆-C₁₀ aralkyl acyl, C₆-C₁₀ alkylaryl acyl, aryl, C₃-C₁₀ heterocyclyl, heteroaryl, optionally substituted C₃-C₁₀cycloalkyl, —PO₃WY, —CH₂PO₄WY, —CH₂PO₄Z or —PO₃Z;
n is an integer of 0, 1, 2, 3, or 4; and
p is an integer of 0, 1, 2 or 3.

20. The compound of claim 1 of Formula XXI: 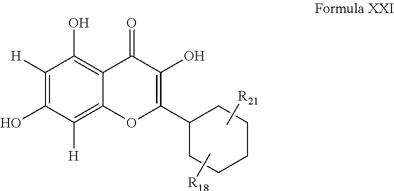
wherein R₁₈ and R₂₁ are independently hydrogen, hydroxyl, carboxaldehyde, amine, C₁-C₁₀ alkyl, C₂-C₁₀ alkynyl, C₂-C₁₀ alkenyl, carboxyl, carbohydrate, ester, acyloxy, nitro, halogen, C₁-C₁₀ aliphatic acyl, C₆-C₁₀ aromatic acyl, C₆-C₁₀ aralkyl acyl, C₆-C₁₀ alkylaryl acyl, alkoxy, alkyl, phosphate, aryl, heteroaryl, C₃-C₁₀ heterocyclic, C₃-C₁₀cycloalkyl, —OPO₃WY, —OCH₂PO₄WY, —OCH₂PO₄Z or —OPO₃Z;
R₂₀ is hydrogen, C₁-C₁₀ alkyl, C₂-C₁₀ alkynyl, C₂-C₁₀ alkenyl, carbohydrate, C₁-C₁₀ aliphatic acyl, C₆-C₁₀ aromatic acyl, C₆-C₁₀ aralkyl acyl, C₆-C₁₀ alkylaryl acyl, aryl, C₃-C₁₀ heterocyclyl, heteroaryl, optionally substituted C₃-C₁₀cycloalkyl, —PO₃WY, —CH₂PO₄WY, —CH₂PO₄Z or —PO₃Z.

21. The compound of claim 1 of Formula XXII: 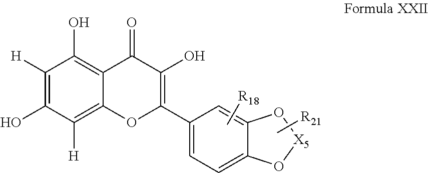
wherein X₅ is a C₁ to C₄ group, optionally interrupted by O, S, NR₂₃, or NR₂₃R₂₃ as valency permits, forming a ring which is aromatic or nonaromatic;
wherein R₁₈ and R₂₁ are independently hydrogen, hydroxyl, carboxaldehyde, amine, C₁-C₁₀ alkyl, C₂-C₁₀ alkynyl, C₂-C₁₀ alkenyl, carboxyl, carbohydrate, ester, acyloxy, nitro, halogen, C₁-C₁₀ aliphatic acyl, C₆-C₁₀ aromatic acyl, C₆-C₁₀ aralkyl acyl, C₆-C₁₀ alkylaryl acyl, alkoxy, alkyl, phosphate, aryl, heteroaryl, C₃-C₁₀ heterocyclic, C₃-C₁₀cycloalkyl, —OPO₃WY, —OCH₂PO₄WY, —OCH₂PO₄Z or —OPO₃Z;
each instance of R₂₃ is independently hydrogen, C₁-C₁₀ alkyl, C₂-C₁₀ alkynyl, C₂-C₁₀ alkenyl, carbohydrate, acyloxy, C₁-C₁₀ aliphatic acyl, C₆-C₁₀ aromatic acyl, C₆-C₁₀ aralkyl acyl, C₆-C₁₀ alkylaryl acyl, alkoxy, aryl, heteroaryl, C₅-C₁₀heterocycyl, C₃-C₁₀cycloalkyl, —PO₃WY, —CH₂PO₄WY, —CH₂PO₄Z or —PO₃Z; and
wherein when X₅ is a C, group, at least one of R₁₈ and R₂₂ is not hydrogen.

22. The compound of claim 1 of Formula XXIII: 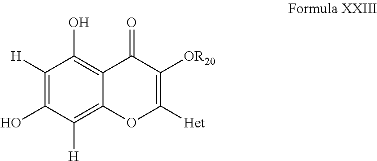
R₂₀ is hydrogen, C₁-C₁₀ alkyl, C₂-C₁₀ alkynyl, C₂-C₁₀ alkenyl, carbohydrate, C₁-C₁₀ aliphatic acyl, C₆-C₁₀ aromatic acyl, C₆-C₁₀ aralkyl acyl, C₆-C₁₀ alkylaryl acyl, aryl, C₃-C₁₀ heterocyclyl, heteroaryl, optionally substituted C₃-C₁₀cycloalkyl, —PO₃WY, —CH₂PO₄WY, —CH₂PO₄Z or —PO₃Z;
Het is a 3 to 10 membered optionally substituted monocyclic or bicyclic heteroaromatic or heteroalicyclic ring system containing 1, 2, 3, 4, or 5 heteroatoms selected from the group of O, S, and N, with the proviso that no two adjacent ring atoms are O or S, wherein the ring system is unsaturated, partially unsaturated or saturated, wherein any number of the ring atoms have substituents as valency permits which are hydrogen, hydroxyl, carboxaldehyde, alkylcarboxaldehyde, imino, C₁-C₁₀ alkyl, C₁-C₁₀ alkynyl, C₁-C₁₀ alkenyl, carboxyl, carbohydrate, acyloxy, nitro, halogen, C₁-C₁₀ aliphatic acyl, C₅-C₁₀ aromatic acyl, C₆-C₁₀ aralkyl acyl, C₆-C₁₀ alkylaryl acyl, alkoxy, amine, aryl, heteroaryl, C₅-C₁₀heterocyclyl, C₅-C₁₀cycloalkyl, —OPO₃WY, —OCH₂PO₄WY, —OCH₂PO₄Z or —OPO₃Z.

23. (canceled)

24. A compound of Formula IV, V, or VI: 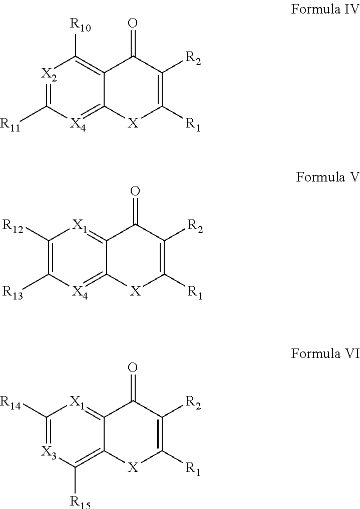
and its pharmaceutically acceptable salts thereof, wherein
X is O, S, or NR′ □wherein R′ is hydrogen, C₁-C₁₀ alkyl, C₂-C₁₀ alkynyl, C₂-C₁₀ alkenyl, C₁-C₁₀ aliphatic acyl, C₆-C₁₀ aromatic acyl, C₆-C₁₀ aralkyl acyl, C₆-C₁₀alkylaryl acyl, aryl, C₃-C₁₀heterocyclyl, heteroaryl, or C₃-C₁₀cycloalkyl;
R₁, and R₂ are independently hydrogen, hydroxyl, C₁-C₁₀ alkyl, C₂-C₁₀ alkynyl, C₂-C₁₀ alkenyl, carboxyl, carbohydrate, ester, acyloxy, nitro, halogen, C₁-C₁₀ aliphatic acyl, C₆-C₁₀ aromatic acyl, C₆-C₁₀ aralkyl acyl, C₆-C₁₀ alkylaryl acyl, alkoxy, amine, aryl, C₄-C₁₀ heterocyclyl, heteroaryl, C₃-C₁₀cycloalkyl, —OPO₃WY, —OCH₂PO₄WY, —OCH₂PO₄Z or —OPO₃Z;
each of X₁, X₂, X₃ and X₄ are independently CR₅, O, S, or N, with the proviso that at least one of X₁, X₂, X₃ and X₄ is N;
each instance of R₅ is independently hydrogen, hydroxyl, carboxaldehyde, amino, C₁-C₁₀ alkyl, C₂-C₁₀ alkynyl, C₂-C₁₀ alkenyl, carboxyl, carbohydrate, ester, acyloxy, nitro, halogen, C₁-C₁₀ aliphatic acyl, C₆-C₁₀ aromatic acyl, C₆-C₁₀ aralkyl acyl, C₆-C₁₀ alkylaryl acyl, alkoxy, amine, aryl, C₃-C₁₀ heterocyclyl, heteroaryl, C₃-C₁₀cycloalkyl, —OPO₃WY, —OCH₂PO₄WY, —OCH₂PO₄Z or —OPO₃Z;
R₁₀, R₁₁, R₁₂, R₁₃, R₁₄ and R₁₅ are independently hydrogen, hydroxyl, carboxaldehyde, amino, C₁-C₁₀ alkyl, C₂-C₁₀ alkynyl, C₂-C₁₀ alkenyl, carboxyl, carbohydrate, ester, acyloxy, nitro, halogen, C₁-C₁₀ aliphatic acyl, C₆-C₁₀ aromatic acyl, C₆-C₁₀ aralkyl acyl, C₆-C₁₀ alkylaryl acyl, alkoxy, amine, aryl, C₃-C₁₀ heterocyclyl, heteroaryl, C₃-C₁₀cycloalkyl, —OPO₃WY, —OCH₂PO₄WY, —OCH₂PO₄Z or —OPO₃Z;
W and Y are independently hydrogen, methyl, ethyl, alkyl, carbohydrate, or a cation, and Z is a multivalent cation.

25-27. (canceled)

28. The compound of claim 24 of Formula XXIV or Formula XXV: 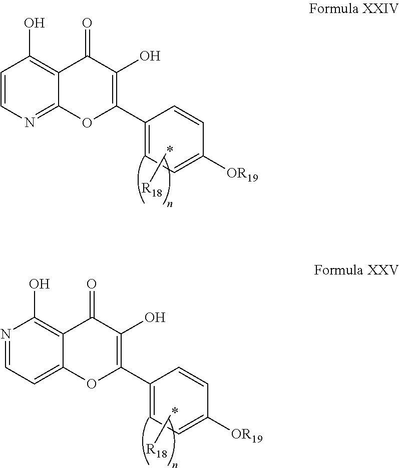
wherein each instance of R₁₈ is independently hydrogen, hydroxyl, carboxaldehyde, amine, C₁-C₁₀ alkyl, C₂-C₁₀ alkynyl, C₂-C₁₀ alkenyl, carboxyl, carbohydrate, ester, acyloxy, nitro, halogen, C₁-C₁₀ aliphatic acyl, C₆-C₁₀ aromatic acyl, C₆-C₁₀ aralkyl acyl, C₆-C₁₀ alkylaryl acyl, alkoxy, alkyl, phosphate, aryl, heteroaryl, C₃-C₁₀heterocyclic, C₃-C₁₀cycloalkyl, —OPO₃WY, —OCH₂PO₄WY, —OCH₂PO₄Z or —OPO₃Z;
R₁₉ is hydrogen, C₁-C₁₀ alkyl, C₂-C₁₀ alkynyl, C₂-C₁₀ alkenyl, carbohydrate, C₁-C₁₀ aliphatic acyl, C₆-C₁₀ aromatic acyl, C₆-C₁₀ aralkyl acyl, C₆-C₁₀ alkylaryl acyl, aryl, C₃-C₁₀ heterocyclyl, heteroaryl, optionally substituted C₃-C₁₀cycloalkyl, —PO₃WY, —CH₂PO₄WY, —CH₂PO₄Z or —PO₃Z; and
n is an integer of 0, 1, 2, 3, or 4.

29. The compound of claim 24 of Formula XXVI, Formula XXVII, or Formula XXVIII: 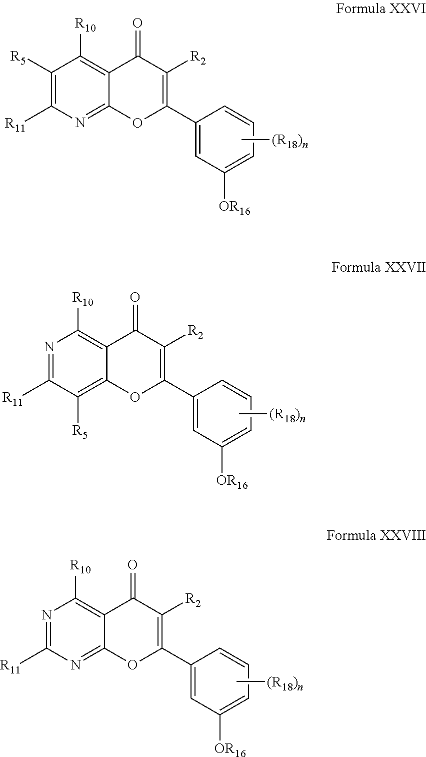
wherein R₁₆ is hydrogen, C₁-C₁₀ alkyl, C₂-C₁₀ alkynyl, C₂-C₁₀ alkenyl, carbohydrate, C₁-C₁₀ aliphatic acyl, C₆-C₁₀ aromatic acyl, C₆-C₁₀ aralkyl acyl, C₆-C₁₀ alkylaryl acyl, aryl, C₃-C₁₀ heterocyclyl, heteroaryl, C₃-C₁₀cycloalkyl, —PO₃WY, —CH₂PO₄WY, —CH₂PO₄Z or —PO₃Z;
each instance of R₁₈ is independently hydrogen, hydroxyl, carboxaldehyde, amine, C₁-C₁₀ alkyl, C₂-C₁₀ alkynyl, C₂-C₁₀ alkenyl, carboxyl, carbohydrate, ester, acyloxy, nitro, halogen, C₁-C₁₀ aliphatic acyl, C₆-C₁₀ aromatic acyl, C₆-C₁₀ aralkyl acyl, C₆-C₁₀ alkylaryl acyl, alkoxy, alkyl, phosphate, aryl, heteroaryl, C₃-C₁₀heterocyclic, C₃-C₁₀cycloalkyl, —OPO₃WY, —OCH₂PO₄WY, —OCH₂PO₄Z or —OPO₃Z; and
n is an integer of 0, 1, 2, 3, or 4.

30. The compound of claim 24 of Formula XXIX, Formula XXX, or Formula XXXI; 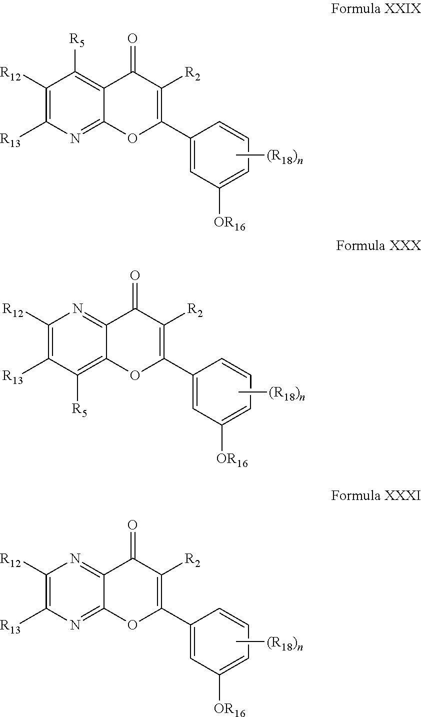
wherein R₁₆ is hydrogen, C₁-C₁₀ alkyl, C₂-C₁₀ alkynyl, C₂-C₁₀ alkenyl, carbohydrate, C₁-C₁₀ aliphatic acyl, C₆-C₁₀ aromatic acyl, C₆-C₁₀ aralkyl acyl, C₆-C₁₀ alkylaryl acyl, aryl, C₃-C₁₀ heterocyclyl, heteroaryl, C₃-C₁₀cycloalkyl, —PO₃WY, —CH₂PO₄WY, —CH₂PO₄Z or —PO₃Z;
each instance of R₁₈ is independently hydrogen, hydroxyl, carboxaldehyde, amine, C₁-C₁₀ alkyl, C₂-C₁₀ alkynyl, C₂-C₁₀ alkenyl, carboxyl, carbohydrate, ester, acyloxy, nitro, halogen, C₁-C₁₀ aliphatic acyl, C₆-C₁₀ aromatic acyl, C₆-C₁₀ aralkyl acyl, C₆-C₁₀ alkylaryl acyl, alkoxy, alkyl, phosphate, aryl, heteroaryl, C₃-C₁₀heterocyclic, C₃-C₁₀cycloalkyl, —OPO₃WY, —OCH₂PO₄WY, —OCH₂PO₄Z or —OPO₃Z; and
n is an integer of 0, 1, 2, 3, or 4.

31. The compound of claim 24 of Formula XXXII, Formula XXXIII, or Formula XXXIV: 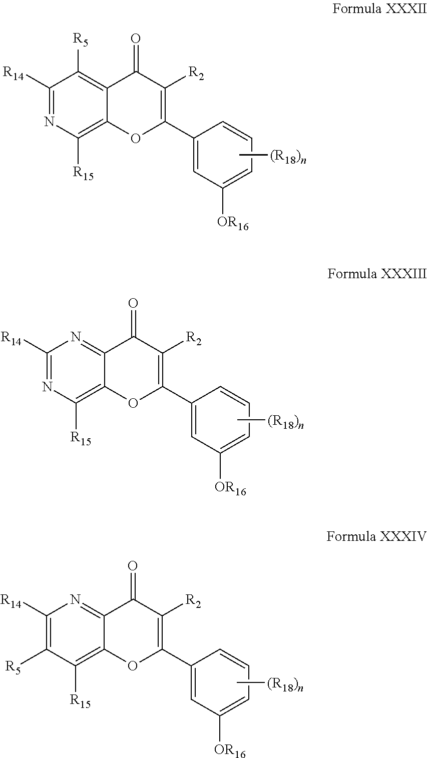
wherein R₁₆ is hydrogen, C₁-C₁₀ alkyl, C₂-C₁₀ alkynyl, C₂-C₁₀ alkenyl, carbohydrate, C₁-C₁₀ aliphatic acyl, C₆-C₁₀ aromatic acyl, C₆-C₁₀ aralkyl acyl, C₆-C₁₀ alkylaryl acyl, aryl, C₃-C₁₀ heterocyclyl, heteroaryl, C₃-C₁₀cycloalkyl, —PO₃WY, —CH₂PO₄WY, —CH₂PO₄Z or —PO₃Z;
each instance of R₁₈ is independently hydrogen, hydroxyl, carboxaldehyde, amine, C₁-C₁₀ alkyl, C₂-C₁₀ alkynyl, C₂-C₁₀ alkenyl, carboxyl, carbohydrate, ester, acyloxy, nitro, halogen, C₁-C₁₀ aliphatic acyl, C₆-C₁₀ aromatic acyl, C₆-C₁₀ aralkyl acyl, C₆-C₁₀ alkylaryl acyl, alkoxy, alkyl, phosphate, aryl, heteroaryl, C₃-C₁₀heterocyclic, C₃-C₁₀cycloalkyl, —OPO₃WY, —OCH₂PO₄WY, —OCH₂PO₄Z or —OPO₃Z; and
n is an integer of 0, 1, 2, 3, or 4.

32-51. (canceled)
